(19) United States
(12) Patent Application Publication
(10) Pub. No.: US 2026/0132185 A1
(43) Pub. Date: May 14, 2026

(54) MULTI-CHAIN CHIMERIC POLYPEPTIDES AND USES THEREOF

(71) Applicant: HCW Biologics, Inc., Miramar, FL (US)

(72) Inventor: Hing C. Wong, Miramar, FL (US)

(73) Assignee: HCW Biologics, Inc., Miramar, FL (US)

(21) Appl. No.: 19/304,367

(22) Filed: Aug. 19, 2025

Related U.S. Application Data
(60) Provisional application No. 63/684,719, filed on Aug. 19, 2024.
(60) Provisional application No. 63/744,832, filed on Jan. 13, 2025.
(60) Provisional application No. 63/830,429, filed on Jun. 25, 2025.

(51) Int. Cl. C07K 14/71; A61K 38/00; A61P 35/00; C07K 14/54; C07K 14/715; C07K 16/28; C07K 16/36; C12N 5/078

(52) U.S. Cl. CPC C07K 14/71; A61P 35/00; C07K 14/54; C07K 14/5418; C07K 14/5443; C07K 14/7155; C07K 16/2809; C07K 16/2818; C07K 16/36; C12N 5/0634; A61K 38/00; C07K 2317/24; C07K 2317/622; C07K 2317/92; C12N 2501/15; C12N 2501/2307; C12N 2501/2315; C12N 2501/2321

(57) ABSTRACT

Provided herein are multi-chain chimeric polypeptides that include: (a) a first chimeric polypeptide including a first target-binding domain a soluble T cell receptor β-chain constant region (TRβC) domain, and a first domain of a pair of affinity domains; and (b) a second chimeric polypeptide including a second domain of a pair of affinity domains and a second target-binding domain, where the first chimeric polypeptide and the second chimeric polypeptide associate through the binding of the first domain and the second domain of the pair of affinity domains. Also provided here are methods of using these multi-chain chimeric polypeptides and nucleic acids encoding these multi-chain chimeric polypeptides.

### CROSS-REFERENCE TO RELATED APPLICATIONS

This application claims priority to: U.S. Provisional Patent Application No. 63/684,719, filed Aug. 19, 2024; U.S. Provisional Patent Application No. 63/744,832, filed Jan. 13, 2025; and U.S. Provisional Patent Application No. 63/830,429, filed Jun. 25, 2025, each of which is herein incorporated by reference in its entirety.

### SEQUENCE LISTING

This application contains a Sequence Listing that has been submitted electronically as an XML file named “47039-0040001_SL.XML.” The XML file, created on Aug. 18, 2025, is 255,711 bytes in size. The material in the XML file is hereby incorporated by reference in its entirety.

### TECHNICAL FIELD

The present disclosure relates to the field of biotechnology, and more specifically, to target-binding molecules.

### BACKGROUND

The T cell receptor (TCR) is expressed by the majority of mature T cell lymphomas (and ˜30% of T cell acute lymphoblastic leukemias). The TCR comprises a heterodimeric protein complex of two chains, TCRα and TCRβ. An ancestral duplication of the human β-chain constant gene results in the expression of one of two highly homologous chains, T cell receptor β-chain constant (TRβC) domains 1 and 2, in a mutually exclusive manner, following TCR locus rearrangement.

Human TRβC1 and TRβC2 isoforms differ by only four amino acid mutations in the extracellular domain. Otherwise, the sequence is identical between the two isoforms and the folded structure remains largely unchanged. While healthy T cells randomly express either TRβC1 or TRβC2, clonally expanded mature T cell lymphomas express one of them exclusively.

### SUMMARY

The present invention is based on the discovery that T-cell receptor β-chain constant region domains 1 or 2 (TRβC1 or TRβC2) can be used as a scaffold for chimeric polypeptides including a target-binding domain. Based on this discovery provided herein are multi-chain chimeric polypeptides that include: (a) a first chimeric polypeptide comprising: (i) a first target-binding domain; (ii) a soluble T cell receptor β-chain constant region (TRβC) domain; and (iii) a first domain of a pair of affinity domains; and (b) a second chimeric polypeptide comprising: (i) a second domain of a pair of affinity domains; and (ii) a second target-binding domain, wherein the first chimeric polypeptide and the second chimeric polypeptide associate through the binding of the first domain and the second domain of the pair of affinity domains. Also provided herein are compositions that include any of the multi-chain chimeric polypeptides described herein, nucleic acids that encode any of the multi-chain chimeric polypeptides described herein, and cells that include any of the nucleic acids that encode any of the multi-chain chimeric polypeptides described herein. Also provided herein are methods of stimulating an immune cell and methods of treating a subject in need thereof that include the use of any of the multi-chain chimeric polypeptides described herein. Also provided herein are methods of producing any of the multi-chain chimeric polypeptides described herein. Additional uses of any of the multi-chain chimeric polypeptides are described herein.

Accordingly, provided herein is a multi-chain chimeric polypeptide comprising: (a) a first chimeric polypeptide comprising: (i) a first target-binding domain; (ii) a soluble T cell receptor β-chain constant region (TRβC) domain; and (iii) a first domain of a pair of affinity domains; and (b) a second chimeric polypeptide comprising: (i) a second domain of a pair of affinity domains; and (ii) a second target-binding domain, wherein the first chimeric polypeptide and the second chimeric polypeptide associate through the binding of the first domain and the second domain of the pair of affinity domains.

In some embodiments, the first target-binding domain and the soluble TRβC domain directly abut each other in the first chimeric polypeptide. In some embodiments, the first chimeric polypeptide further comprises a linker sequence between the first target-binding domain and the soluble TRβC domain in the first chimeric polypeptide. In some embodiments, the soluble TRβC domain and the first domain of the pair of affinity domains directly abut each other in the first chimeric polypeptide. In some embodiments, the first chimeric polypeptide further comprises a linker sequence between the soluble TRβC domain and the first domain of the pair of affinity domains in the first chimeric polypeptide. In some embodiments, the second domain of the pair of affinity domains and the second target-binding domain directly abut each other in the second chimeric polypeptide. In some embodiments, the second chimeric polypeptide further comprises a linker sequence between the second domain of the pair of affinity domains and the second target-binding domain in the second chimeric polypeptide. In some embodiments, the first chimeric polypeptide further comprises one or more additional target-binding domains. In some embodiments, the first domain of the pair of affinity domains and one of the one or more additional target-binding domains directly abut each other in the first chimeric polypeptide. In some embodiments, the first chimeric polypeptide further comprises a linker sequence between the first domain of the pair of affinity domains and one of the one or more additional target-binding domains in the first chimeric polypeptide. In some embodiments, the second chimeric polypeptide further comprises one or more additional target-binding domains. In some embodiments, the second domain of the pair of affinity domains and one of the one or more additional target-binding domains directly abut each other in the second chimeric polypeptide. In some embodiments, the second chimeric polypeptide further comprises a linker sequence between the second domain of the pair of affinity domains and one of the one or more additional target-binding domains in the second chimeric polypeptide.

In some embodiments, the one or more additional target-binding domains bind specifically to a target selected from the group consisting of: CD3, Tissue Factor (CD142), PD-1, CD26, and CD36. In some embodiments, the one or more additional target-binding domains comprises an anti-PD-1 diabody or scFv, e.g., pembrolizumab. In some embodiments, the first target-binding domain and the second target-binding domain bind specifically to the same antigen. In some embodiments, the first target-binding domain and the second target-binding domain bind specifically to different antigens.

In some embodiments, one or both of the first target-binding domain and the second target-binding domain is an antigen-binding domain. In some embodiments, one or both of the first target-binding domain and the second target-binding domain bind specifically to a target selected from the group consisting of: DDL-3, GPC3, CD73, CECAM5/6, Tissue Factor (CD142), CD16a, CD28, CD3, CD33, CD20, CD19, CD22, CD123, IL-1R, IL-1, VEGF, IL-6R, IL-4, IL-10, PDL-1, TIGIT, PD-1, TIM3, CTLA4, MICA, MICB, IL-6, IL-8, TNFα, CD26a, CD36, ULBP2, CD30, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5/6, uPAR (urokinase plasminogen activator receptor), a UL16-binding protein, HLA-DR, DLL4, TYRO3, AXL, MER, CD122, CD155, PDGFDD, a ligand of TGF-β receptor II (TGF-βRII), a ligand of TGF-βRIII, a ligand of DNAM1, a ligand of NKp46, a ligand of NKp44, a ligand of NKG2D, a ligand of NKp30, a ligand for a scMHCI, a ligand for a scMHCII, a ligand for a scTCR, a ligand of ICOS, a receptor for IL-1, a receptor for IL-2, a receptor for IL-3, a receptor for IL-4, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-DD, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28. In some embodiments, one or both of the first target-binding domain and the second target-binding domain is a soluble interleukin or cytokine protein. In some embodiments, the soluble interleukin, cytokine, or ligand protein is selected from the group consisting of: IL-1, IL-2, IL-3, IL-4, IL-7, IL-8, IL-10, IL-12, IL-15, IL-17, IL-18, IL-21, PDGF-DD, SCF, and FLT3L. In some embodiments, the soluble interleukin, soluble cytokine protein, or soluble cell surface protein is selected from the group consisting of: IL-7 and IL-21. In some embodiments, one or both of the first target-binding domain and the second target-binding domain is a soluble interleukin or cytokine receptor. In some embodiments, the soluble receptor is a soluble TGF-β receptor II (TGF-βRII), a soluble TGF-βRIII, a soluble NKG2D, a soluble NKp30, a soluble NKp44, a soluble NKp46, a soluble DNAM-1, a scMHCI, a scMHCII, a scTCR, a soluble CD155, or a soluble CD28. In some embodiments, the soluble interleukin receptor, the soluble cytokine receptor, or the soluble cell surface receptor is a soluble TGF-βRII. In some embodiments, the first target-binding domain is IL-7 and the second target-binding domain is a soluble TGF-βRII. In some embodiments, the second chimeric polypeptide further comprises an anti-PD-1 antigen-binding domain. In some embodiments, the second chimeric polypeptide further comprises an anti-tissue factor antigen-binding domain. In some embodiments, the first target-binding domain is a soluble TGF-βRII and the second target-binding domain is a soluble TGF-βRII. In some embodiments, the first target-binding domain is IL-7 and the second target-binding domain is a soluble IL-21. In some embodiments, the first target-binding domain is soluble IL-7 and the second target-binding domain is an anti-CD3 antigen-binding domain. In some embodiments, the anti-CD3 antigen-binding domain is a single chain anti-CD3 antigen-binding domain. In some embodiments, the second chimeric polypeptide further comprises an anti-tissue factor antigen-binding domain. In some embodiments, the first target-binding domain is a soluble IL-7 and the second target-binding domain is an anti-PD-1 antigen-binding domain. In some embodiments, the second chimeric polypeptide further comprises a soluble IL-21. In some embodiments, the first target-binding domain is a soluble IL-7 and the second antigen-binding domain is an anti-tissue factor antigen-binding domain. In some embodiments, the second chimeric polypeptide further comprises an anti-CD3 antigen-binding domain. In some embodiments, the first target-binding domain is a soluble TGF-βRII and the second target-binding domain is a soluble anti-tissue factor antigen-binding domain. In some embodiments, the second chimeric polypeptide further comprises an anti-CD16 antigen-binding domain. In some embodiments, the second chimeric polypeptide further comprises an anti-CD3 antigen-binding domain. In some embodiments, the second chimeric polypeptide further comprises an anti-CD16 antigen-binding domain. In some embodiments, the first target-binding domain is a soluble TGF-βRII and the second target-binding domain is an anti-PD-1 antigen-binding domain. In some embodiments, the second chimeric polypeptide further comprises a soluble IL-7. In some embodiments, the first target-binding domain is a soluble TGF-βRII and the second target-binding domain is an anti-CD3 antigen-binding domain. In some embodiments, the second chimeric polypeptide further comprises an anti-tissue factor antigen-binding domain. In some embodiments, the first target-binding domain is an anti-tissue factor antigen-binding domain and the second target-binding domain is an anti-PD-1 antigen-binding domain. In some embodiments, the second chimeric polypeptide further comprises a soluble IL-7.

In some embodiments, the first target-binding domain comprises a sequence that is at least 80% identical to SEQ ID NO: 4. In some embodiments, the second target-binding domain comprises a sequence that is at least 80% identical to SEQ ID NO: 5. In some embodiments, one or both of the first target-binding domain and the second target-binding domain comprises a first sequence that is at least 80% identical to SEQ ID NO: 7 and a second sequence that is at least 80% identical to SEQ ID NO: 7, wherein the first and second sequence are separated by a linker. In some embodiments, one or both of the first target-binding domain and the second target-binding domain comprises a sequence that is at least 80% identical to SEQ ID NO: 9. In some embodiments, one or both of the first target-binding domain and the second target-binding domain comprises a sequence that is at least 80% identical to SEQ ID NO: 51.

In some embodiments, the soluble TRβC domain is a soluble human TRβC domain. In some embodiments, the soluble TRβC domain does not bind to an MHC protein with or without a loaded antigen. In some embodiments, the soluble TRβC domain does not stimulate an immune response in a mammal. In some embodiments, the soluble TRβC domain comprises or consists of a sequence from a wildtype soluble human TRβC. In some embodiments, the soluble TRβC domain comprises a sequence that is at least 80% identical to SEQ ID NO: 1. In some embodiments, the soluble human TRβC domain comprises a sequence that is at least 80% identical to SEQ ID NO: 3.

In some embodiments, the pair of affinity domains is a sushi domain from an alpha chain of human IL-15 receptor (IL-15Rα) and a soluble IL-15. In some embodiments, the pair of affinity domains is selected from the group consisting of: barnase and barnstar, a PKA and an AKAP, adapter/docking tag modules based on mutated RNase I fragments, and SNARE modules based on interactions of the proteins syntaxin, synaptotagmin, synaptobrevin, and SNAP25.

In some embodiments, the first chimeric polypeptide and/or the second chimeric polypeptide further comprises a signal sequence at its N-terminal end.

Also provided herein is a composition comprising any of the multi-chain chimeric polypeptides discussed above. In some embodiments, the composition is a pharmaceutical composition. In some embodiments, a kit comprising at least one dose of any of the compositions discussed above is provided herein.

Also provided herein is a method of inducing or increasing proliferation of an immune cell, the method comprising: contacting an immune cell with an effective amount of any of the multi-chain chimeric polypeptides discussed above or any of the compositions discussed above.

Also provided herein is a method of inducing or increasing proliferation of an immune cell, the method comprising: contacting an immune cell with an effective amount of any of the multi-chain chimeric polypeptides discussed above or any of the compositions discussed above. Also provided herein is a method of inducing differentiation of an immune cell into a memory or memory-like immune cell, the method comprising: contacting an immune cell with an effective amount of any of the multi-chain chimeric polypeptides discussed above or any of the compositions discussed above.

In some embodiments, the immune cell is contacted in vitro. In some embodiments, the immune cell is contacted in vivo. In some embodiments, the immune cell is selected from the group consisting of: an immature thymocyte, a peripheral blood lymphocyte, a naïve T cell, a pluripotent Th cell precursor, a lymphoid progenitor cell, a Treg cell, a Th17 cell, a Th22 cell, a Th9 cell, a Th2 cell, a Th1 cell, a Th3 cell, γδ T cell, an αβ T cell, a tumor-infiltrating T cell, a CD8⁺ T cell, a CD4⁺ T cell, a natural killer T cell, a mast cell, a macrophage, a neutrophil, a dendritic cell, a basophil, an eosinophil, and a natural killer cell. In some embodiments, the CD8⁺ T cell is a progenitor exhausted CD8⁺ T (Tpex) cell, an effector memory CD8⁺ T (Tem) cell, a central memory CD8⁺ T (Tcm) cell, a memory CD8⁺ stem (Tscm) cell, a virtual memory CD8⁺ T (Tvm) cell, an antigen-experienced CD8⁺ T cell, or a terminally-exhausted CD8⁺ T (Tex) cell.

In some embodiments, the immune cell has previously been genetically modified to express a chimeric antigen receptor or a recombinant T-cell receptor.

Also provided herein is a method of killing a cancer cell, an infected cell, or a senescent cell in a subject in need thereof, the method comprising administering to the subject a therapeutically effective amount of any of the multi-chain chimeric polypeptides discussed above or any of the compositions discussed above. Also provided herein is a method of treating a subject in need thereof, the method comprising administering to the subject a therapeutically effective amount of any of the multi-chain chimeric polypeptides discussed above or any of the compositions discussed above.

In some embodiments, the subject has been identified or diagnosed as having a cancer, an aging-related disease or condition, or an infectious disease. In some embodiments, the cancer is selected from the group consisting of: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma. In some embodiments, the aging-related disease or condition is selected from the group consisting of: a senescence-associated skin condition, atopic dermatitis, wrinkles, senile lentigo, age spots, photoaged skin, radiation-induced dermatitis, radiation-induced skin fibrosis/injury, cutaneous wound healing, Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, bronchopulmonary dysplasia, and renal dysfunction. In some embodiments, the infectious disease is infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, and influenza virus.

Also provided herein is a nucleic acid encoding any of the multi-chain chimeric polypeptides discussed above. Some embodiments comprise a vector containing any of the nucleic acids discussed above. In some embodiments, the vector is an expression vector. Some embodiments comprise a cell containing any of the nucleic acids or vectors discussed above.

Also provided herein is a method of producing a multi-chain chimeric polypeptide, the method comprising: culturing the cell discussed above in a culture medium under conditions sufficient to result in the production of the multi-chain chimeric polypeptide; and recovering the multi-chain chimeric polypeptide from the cell and/or the culture medium. In some embodiments, the method comprises producing a multi-chain chimeric polypeptide by the methods discussed above.

Also provided herein is a method of producing a multi-chain chimeric polypeptide, the method comprising: culturing a cell discussed above in a culture medium under conditions sufficient to result in the production of the multi-chain chimeric polypeptide; and recovering the multi-chain chimeric polypeptide from the cell and/or the culture medium. In some embodiments, provided herein is a multi-chain chimeric polypeptide produced by the methods discussed above.

A “multi-chain polypeptide” as used herein to refers to a polypeptide comprising two or more (e.g., three, four, five, six, seven, eight, nine, or ten) protein chains (e.g., at least a first chimeric polypeptide and a second polypeptide), where the two or more proteins chains associate through non-covalent bonds to form a quaternary structure.

As used herein, the term “chimeric” refers to a polypeptide that includes amino acid sequences (e.g., domains) originally derived from two different sources (e.g., two different naturally-occurring proteins, e.g., from the same or different species). For example, a chimeric polypeptide can include domains from at least two different naturally occurring human proteins.

In some examples, a chimeric polypeptide can include a domain that is a synthetic sequence (e.g., an scFv) and a domain that is derived from a naturally-occurring protein (e.g., a naturally-occurring human protein). In some embodiments, a chimeric polypeptide can include at least two different domains that are synthetic sequences (e.g., two different scFvs).

A “T cell receptor β-chain constant region domain” refers to a polypeptide having at least 70% identity (e.g., at least 75% identity, at least 80% identity, at least 85% identity, at least 90% identity, at least 95% identity, at least 99% identity, or 100% identical) to a segment of a constant region of a wildtype mammalian T cell receptor β-chain (e.g., a wildtype human T cell receptor β-chain). Different species have 1 to 5 different genes encoding the T cell receptor β-chain constant region domain. Human and mice have genes encoding the TRβC1 or TRβC2 isoforms. Non-limiting examples of T cell receptor β-chain constant region domains are described herein.

The term “soluble interleukin protein” is used herein to refer to a mature and secreted interleukin protein or a biologically active fragment thereof. In some examples, a soluble interleukin protein can include a sequence that is at least 70% identical, at least 75% identical, at least 80% identical, at least 85% identical, at least 90% identical, at least 95% identical, at least 99% identical, or 100% identical to a wildtype mature and secreted mammalian interleukin protein (e.g., a wildtype human interleukin protein) and retains its biological activity. Non-limiting examples of soluble interleukin proteins are described herein.

The term “soluble cytokine protein” is used herein to refer to a mature and secreted cytokine protein or a biologically active fragment thereof. In some examples, a soluble cytokine protein can include a sequence that is at least 70% identical, at least 75% identical, at least 80% identical, at least 85% identical, at least 90% identical, at least 95% identical, at least 99% identical, or 100% identical to a wildtype mature and secreted mammalian cytokine protein (e.g., a wildtype human cytokine protein) and retains its biological activity. Non-limiting examples of soluble cytokine proteins are described herein.

The term “soluble interleukin receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble interleukin receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype interleukin receptor and retains its ability to specifically bind to one or more of its natural ligands, but lacks its transmembrane domain (and optionally, further lacks its intracellular domain). Non-limiting examples of soluble interleukin receptors are described herein.

The term “soluble cytokine receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble cytokine receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype cytokine receptor and retains its ability to specifically bind to one or more of its natural ligands, but lacks its transmembrane domain (and optionally, further lacks its intracellular domain). Non-limiting examples of soluble cytokine receptors are described herein.

An “antigen-binding domain” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, and multi-specific antibodies. One example of an antigen-binding domain is an antigen-binding domain formed by a V_{H}-V_{L} or a V_{L}-V_{H}. Another example of an antigen-binding domain is an antigen-binding domain formed by a V_{H}-V_{L} dimer or V_{L}-V_{H} dimer. Additional examples of an antibody are described herein. Additional examples of an antibody are known in the art.

The term “epitope” means a portion of an antigen that specifically binds to an antigen-binding domain. Epitopes can, e.g., consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding.

Methods for identifying an epitope to which an antigen-binding domain binds are known in the art.

An “Fv” fragment includes a non-covalently-linked dimer of one heavy chain variable domain and one light chain variable domain.

A “Fab” fragment includes, the constant domain of the light chain and the first constant domain (C_{H1}) of the heavy chain, in addition to the heavy and light chain variable domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an antigen-binding domain and an antigen or epitope. The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining the affinity for an antigen-binding domain and its corresponding antigen or epitope are known in the art.

The term “pair of affinity domains” is two different protein domain(s) that bind specifically to each other with a K_{D} of less than of less than 1×10⁻⁷ M (e.g., less than 1×10⁻⁸ M, less than 1×10⁻⁹ M, less than 1×10⁻¹⁰ M, or less than 1×10⁻¹¹ M). In some examples, a pair of affinity domains can be a pair of naturally-occurring proteins. In some embodiments, a pair of affinity domains can be a pair of synthetic proteins. Non-limiting examples of pairs of affinity domains are described herein.

An “immune effector cell” refers to a cell of the immune system of a mammal that is capable, directly or indirectly, of recognizing and/or causing cytostasis or cell death of a pathogenic cell (e.g., a cancer cell) in the mammal. Non-limiting examples of immune effector cells include macrophages, T-lymphocytes (e.g., cytotoxic T-lymphocytes and T-helper cells), natural killer cells, neutrophils, monocytes, and eosinophils. Additional examples of immune effector cells are known in the art.

The term “treatment” means to ameliorate at least one symptom of a disorder. In some examples, the disorder being treated is cancer and to ameliorate at least one symptom of cancer includes reducing aberrant proliferation, gene expression, signaling, translation, and/or secretion of factors. Generally, the methods of treatment include administering a therapeutically effective amount of composition that reduces at least one symptom of a disorder to a subject who is in need of, or who has been determined to be in need of such treatment.

The term “subject” refers to any mammal. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine or primate animals) may be employed.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A and 1B are schematics of an exemplary multi-chain chimeric polypeptide: (i) a first chimeric polypeptide including a first target-binding domain (A in FIG. 1), a soluble T cell receptor (TCR) β-chain constant region (TRβC) domain, a first domain of an affinity pair of domains (B in FIG. 1B); and (ii) a second chimeric polypeptide including a second domain of an affinity pair of domains (D in FIG. 1B) and a second target-binding domain (C in FIG. 1). The schematic in FIG. 1A depicts the association of the first and the second chimeric polypeptides through the pair of affinity domains. The schematic in FIG. 1B shows the order of the domains in the first and second chimeric polypeptides.

FIGS. 2A and 2B are schematics of an exemplary multi-chain chimeric polypeptide: (i) a first chimeric polypeptide including a first target-binding domain (A in FIG. 2B), a soluble TRβC domain, a first domain of an affinity pair of domains (B in FIG. 2B); and (ii) a second chimeric polypeptide including a second domain of an affinity pair of domains (D in FIG. 2B), a second target-binding domain (C in FIG. 2B), and an additional target-binding domain (E in FIG. 2B). The schematic in FIG. 2A depicts the association of the first and the second chimeric polypeptides through the pair of affinity domains. The schematic in FIG. 2B shows the order of the domains in the first and second chimeric polypeptides.

FIGS. 3A and 3B are schematics of an exemplary multi-chain chimeric polypeptide: (i) a first chimeric polypeptide including a first target-binding domain (A in FIG. 3B), a soluble human TRβC1 domain (hTRβC1), a first domain of an affinity pair of domains (soluble interleukin IL-15; IL-15 in FIG. 3B); and (ii) a second chimeric polypeptide including a second domain of an affinity pair of domains (IL-15 receptor alpha sushi domain; Rain FIG. 3B), a second target-binding domain (C in FIG. 3B), and an additional target-binding domain (a diabody; Diabody in FIG. 3B). The schematic in FIG. 3A depicts the association of the first and the second chimeric polypeptides through the pair of affinity domains. The schematic in FIG. 3B shows the order of the domains in the first and second chimeric polypeptides.

FIGS. 4A and 4B are schematic diagrams of exemplary IL-7/hTRβC1/IL-15 and TGFβRII/IL-15RαSu/Anti-PD-1 diabody fusion proteins resulting in IL-7/hTRβC1/IL-15: TGFβRII/IL-15RαSu/Anti-PD-1 diabody complex. The schematic in FIG. 4A depicts the association of the first and the second chimeric polypeptides through the pair of affinity domains. The schematic in FIG. 4B shows the order of the domains in the first and second chimeric polypeptides.

FIGS. 5A and 5B are schematic diagrams of exemplary IL-7/hTRβC1/IL-15 and TGFβRII/IL-15RαSu fusion proteins resulting in IL-7/hTRβC1/IL-15: TGFβRII/IL-15RαSu complex. The schematic in FIG. 5A depicts the association of the first and the second chimeric polypeptides through the pair of affinity domains. The schematic in FIG. 5B shows the order of the domains in the first and second chimeric polypeptides.

FIGS. 6A and 6B are schematic diagrams of exemplary IL-7/hTRβC1/IL-15 and TGFβRII/IL-15RαSu/Anti-Tissue Factor (Anti-TF) diabody fusion proteins resulting in IL-7/hTRβC1/IL-15: TGFβRII/IL-15RαSu/Anti-Tissue Factor (Anti-TF) diabody complex. The schematic in FIG. 6A depicts the association of the first and the second chimeric polypeptides through the pair of affinity domains. The schematic in FIG. 6B shows the order of the domains in the first and second chimeric polypeptides.

FIGS. 7A and 7B are schematic diagrams of exemplary TGFβRII/hTRβC1/IL-15 and TGFβRII/IL-15RαSu fusion proteins resulting in TGFβRII/hTRβC1/IL-15:TGFβRII/IL-15RαSu complex. The schematic in FIG. 7A depicts the association of the first and the second chimeric polypeptides through the pair of affinity domains. The schematic in FIG. 7B shows the order of the domains in the first and second chimeric polypeptides.

FIGS. 8A and 8B are schematic diagrams of exemplary IL-7/hTRβC1/IL-15 and IL-21/IL-15RαSu fusion proteins resulting in exemplary IL-7/hTRβC1/IL-15:IL-21/IL-15RαSu complex. The schematic in FIG. 8A depicts the association of the first and the second chimeric polypeptides through the pair of affinity domains. The schematic in FIG. 8B shows the order of the domains in the first and second chimeric polypeptides.

FIGS. 9A and 9B are schematic diagrams of exemplary IL-7/hTRβC1/IL-15 and scCD3 antibody/IL-15RαSu fusion proteins resulting in exemplary IL-7/hTRβC1/IL-15: scCD3 antibody/IL-15RαSu complex. The schematic in FIG. 9A depicts the association of the first and the second chimeric polypeptides through the pair of affinity domains. The schematic in FIG. 9B shows the order of the domains in the first and second chimeric polypeptides.

FIG. 10A shows an increase in IFN-7 in day 5 supernatant from human PBMC stimulated with IL-7/hTRβC1/IL-15:IL-21/IL-15RαSu complex in vitro.

FIG. 10B is a plot showing minimal expression of IL-6 in day 5 supernatant from human PBMC stimulated with IL-7/hTRβC1/IL-15:IL-21/IL-15RαSu complex in vitro.

FIG. 11 shows IL-7/hTRβC1/IL-15:IL-21/IL-15RαSu complex treatment slows B16F10 tumor growth in C57BL/6 mice.

FIG. 12A shows results of a reduced SDS-PAGE analysis of IL-7/hTRβC1/IL-15:IL-21/IL-15RαSu complex compared with 7t15-21s.

FIG. 12B shows a comparison of chromatographic profiles of IL-7/hTRβC1/IL-15:IL-21/IL-15RαSu complex and 7t15-21s.

FIG. 13A shows the biological activity of IL-15 in HEK-Blue IL-2 reporter cells stimulated with 7t15-21s or IL-7/hTRβC1/IL-15:IL-21/IL-15RαSu complex.

FIG. 13B shows biological activity of IL-7 in HEK-Blue IL-7 reporter cells stimulated with 7t15-21s or IL-7/hTRβC1/IL-15:IL-21/IL-15RαSu complex.

FIG. 14 shows biological activity of IL-21 in B9 cells stimulated with 7t15-21s or IL-7/hTRβC1/IL-15:IL-21/IL-15RαSu complex.

FIG. 15A shows a schematic of the experimental study related to FIGS. 15B-15D.

FIG. 15B shows spleen weight in mice treated with 7t15-21s or IL-7/hTRβC1/IL-15:IL-21/IL-15RαSu complex as compared to PBS control.

FIG. 15C shows the number per μL of CD4⁺ T cells, CD8⁺ T cells, and NK cells from blood cells in mice treated with 7t15-21s or IL-7/hTRβC1/IL-15:IL-21/IL-15RαSu complex as compared to PBS control.

FIG. 15D shows the percentage of CD4⁺ T cells, CD8⁺ T cells, and NK cells from splenocytes in mice treated with 7t15-21s or IL-7/hTRβC1/IL-15:IL-21/IL-15RαSu complex as compared to PBS control.

FIG. 16A shows a schematic of the experimental study related to FIGS. 16B-16E.

FIG. 16B shows the mean fluorescent intensity (MFI) of granzyme B in blood cells from mice treated with 7t15-21s or IL-7/hTRβC1/IL-15:IL-21/IL-15RαSu complex as compared to PBS control.

FIG. 16C shows the mean fluorescent intensity (MFI) of Ki67 in blood cells from mice treated with 7t15-21s or IL-7/hTRβC1/IL-15:IL-21/IL-15RαSu complex as compared to PBS control.

FIG. 16D shows the mean fluorescent intensity (MFI) of CD44 in blood cells from mice treated with 7t15-21s or IL-7/hTRβC1/IL-15:IL-21/IL-15RαSu complex as compared to PBS control.

FIG. 16E shows the mean fluorescent intensity (MFI) of TIM3 in blood cells from mice treated with 7t15-21s or IL-7/hTRβC1/IL-15:IL-21/IL-15RαSu complex as compared to PBS control.

FIG. 17A shows a schematic of the experimental study related to FIGS. 17B-17E.

FIG. 17B shows the mean fluorescent intensity (MFI) of granzyme B in splenocytes from mice treated with 7t15-21s or IL-7/hTRβC1/IL-15:IL-21/IL-15RαSu complex as compared to PBS control.

FIG. 17C shows the mean fluorescent intensity (MFI) of Ki67 in splenocytes from mice treated with 7t15-21s or IL-7/hTRβC1/IL-15:IL-21/IL-15RαSu complex as compared to PBS control.

FIG. 17D shows the mean fluorescent intensity (MFI) of CD44 in splenocytes from mice treated with 7t15-21s or IL-7/hTRβC1/IL-15:IL-21/IL-15RαSu complex as compared to PBS control.

FIG. 17E shows the mean fluorescent intensity (MFI) of TIM3 in splenocytes from mice treated with 7t15-21s or IL-7/hTRβC1/IL-15:IL-21/IL-15RαSu complex as compared to PBS control.

FIGS. 18A-18B show an increase in lymphocytes (CD3+) proliferation in human PBMCs stimulated with IL-7/hTRβC1/IL-15:IL-21/IL-15RαSu complex in vitro.

FIG. 19 shows IL-7/hTRβC1/IL-15:IL-21/IL-15RαSu complex treatment in combination with BCG increases immune cell counts in bladder of C57BL/6 mice in vivo.

FIG. 20A shows an increase in expression of IFN-γ in NK cells treated with IL-7/hTRβC1/IL-15:IL-21/IL-15RαSu complex.

FIG. 20B shows increase in expression of IFN-γ in CD8+ cells treated with IL-7/hTRβC1/IL-15:IL-21/IL-15RαSu complex.

FIG. 21A shows increase in expression of granzyme-β in NK cells treated with IL-7/hTRβC1/IL-15:IL-21/IL-15RαSu complex.

FIG. 21B shows increase in expression of granzyme-β in CD8⁺ cells treated with IL-7/hTRβC1/IL-15:IL-21/IL-15RαSu complex.

FIG. 22A shows increase in expression of IFN-γ in NK cells treated with IL-7/hTRβC1/IL-15:IL-21/IL-15RαSu complex.

FIG. 22B shows increase in expression of IFN-γ in human CD8⁺ cells treated with IL-7/hTRβC1/IL-15:IL-21/IL-15RαSu complex.

FIG. 23A shows increase in expression of granzyme-β in NK cells treated with IL-7/hTRβC1/IL-15:IL-21/IL-15RαSu complex.

FIG. 23B shows increase in expression of granzyme-β in CD8+ cells treated with IL-7/hTRβC1/IL-15:IL-21/IL-15RαSu complex.

FIG. 24 shows characterization of TGFβRII/hTRβC1/IL-15:TGFβRII/IL-15RαSu complex by SDS-PAGE (left) and size exclusion chromatography (right).

FIG. 25A shows component characterization of TGFβRII/hTRβC1/IL-15:TGFβRII/IL-15RαSu complex where IL-15 and TGFβ are detected by corresponding antibodies.

FIG. 25B shows component characterization of TGFβRII/hTRβC1/IL-15:TGFβRII/IL-15RαSu complex confirming biological activity with reporter cell lines using TGFβ as a control.

FIG. 25C shows component characterization of TGFβRII/hTRβC1/IL-15:TGFβRII/IL-15RαSu complex confirming biological activity with reporter cell line using IL-15 as a control.

FIG. 26A shows spleen weight in mice treated with TGFβRII/hTRβC1/IL-15:TGFβRII/IL-15RαSu complex as compared to PBS control in C57BL/6 mice.

FIG. 26B shows the number per μL of CD4⁺ T cells, CD8⁺ T cells, and NK cells from blood cells in mice treated with TGFβRII/hTRβC1/IL-15:TGFβRII/IL-15RαSu complex as compared to PBS control.

FIG. 26C shows the percentage of CD4⁺ T cells, CD8⁺ T cells, and NK cells from splenocytes in mice treated with TGFβRII/hTRβC1/IL-15:TGFβRII/IL-15RαSu complex as compared to PBS control.

FIG. 27A shows the expression of granzyme β by CD8⁺ T cells and NK cells in blood from mice treated with TGFβRII/hTRβC1/IL-15:TGFβRII/IL-15RαSu complex as compared to PBS control.

FIG. 27B shows the expression of Ki67 by CD8⁺ T cells and NK cells in blood from mice treated with TGFβRII/hTRβC1/IL-15:TGFβRII/IL-15RαSu complex as compared to PBS control.

FIG. 27C shows the expression of granzyme B by CD8⁺ T cells and NK cells in spleen from mice treated with TGFβRII/hTRβC1/IL-15:TGFβRII/IL-15RαSu complex as compared to PBS control.

FIG. 27D shows the expression of Ki67 by CD8⁺ T cells and NK cells in spleen from mice treated with TGFβRII/hTRβC1/IL-15:TGFβRII/IL-15RαSu complex as compared to PBS control.

FIG. 28A shows spleen weight in mice treated with TGFβRII/hTRβC1/IL-15:TGFβRII/IL-15RαSu complex in C57BL/6 mice.

FIG. 28B shows the percentage of CD8⁺ cells and NK cells in spleen from mice treated with TGFβRII/hTRβC1/IL-15:TGFβRII/IL-15RαSu complex in C57BL/6 mice.

FIG. 28C shows the expression of granzyme B by CD8+ T cells and NK cells in spleen from mice treated with TGFβRII/hTRβC1/IL-15:TGFβRII/IL-15RαSu complex.

FIG. 28D shows the expression of Ki67 by CD8+ T cells and NK cells in spleen from mice treated with TGFβRII/hTRβC1/IL-15:TGFβRII/IL-15RαSu complex.

FIG. 29A is a schematic diagram of exemplary IL-7/hTRβC1/IL-15 and αTF scFv/IL-15RαSu/αCD3 scFv fusion proteins resulting in IL-7/hTRβC1/IL-15: αTF scFv/IL-15RαSu/αCD3 scFv complex. The schematic depicts the association of the first and the second chimeric polypeptides through the pair of affinity domains.

FIG. 29B is a schematic diagram of exemplary TGFβRII/hTRβC1/IL-15 and αTF scFv/IL-15RαSu/αCD3 scFv fusion proteins resulting in TGFβRII/hTRβC1/IL-15: αTF scFv/IL-15RαSu/αC3 scFv complex. The schematic depicts the association of the first and the second chimeric polypeptides through the pair of affinity domains.

FIG. 30A shows CD25⁺ expression within CD4⁺ T cell populations inAsPC1 and MIA pancreatic cancer cell lines treated with treatment with TGFβRII/hTRβC1/IL-15: αTF scFv/IL-15RαSu/αCD3 scFv complex at concentrations of 100 nM, 10 nM, 1 nM, and 0.1 nM.

FIG. 30B shows CD69⁺ expression within CD4⁺ T cell populations in AsPC1 and MIA pancreatic cancer cell lines treated with treatment with TGFβRII/hTRβC1/IL-15: αTF scFv/IL-15RαSu/αCD3 scFv complex at concentrations of 100 nM, 10 nM, 1 nM, and 0.1 nM.

FIG. 30C shows CD25⁺ expression within CD8⁺ T cell populations in AsPC1 and MIA pancreatic cancer cell lines treated with treatment with TGFβRII/hTRβC1/IL-15: αTF scFv/IL-15RαSu/αCD3 scFv complex at concentrations of 100 nM, 10 nM, 1 nM, and 0.1 nM.

FIG. 30D shows CD69⁺ expression within CD8⁺ T cell populations in AsPC1 and MIA pancreatic cancer cell lines treated with treatment with TGFβRII/hTRβC1/IL-15: αTF scFv/IL-15RαSu/αCD3 scFv complex at concentrations of 100 nM, 10 nM, 1 nM, and 0.1 nM.

FIG. 31A shows TGFβRII/hTRβC1/IL-15: αTF scFv/IL-15RαSu/αCD3 scFv complex-mediated target cell-T cell conjugation by flow cytometry revealing the crosslinking of CD3+ Jurkat cells and TF+ AsPC-1 cells.

FIG. 31B shows TGFβRII/hTRβC1/IL-15: αTF scFv/IL-15RαSu/αCD3 scFv complex-mediated target cell-T cell cytotoxicity in human PBMCs evaluated through appearance of PI+ dead cells.

FIG. 32 is a schematic diagram of exemplary TGFβRII/hTRβC1/IL-15 and αTF scFv/IL-15RαSu/αCD16 scFv fusion proteins resulting in TGFβRII/hTRβC1/IL-15: αTF scFv/IL-15RαSu/αC16 scFv complex. The schematic depicts the association of the first and the second chimeric polypeptides through the pair of affinity domains.

FIG. 33A shows a schematic of the experimental study related to FIGS. 33B and 33C.

FIG. 33B shows tumor volumes at days indicated comparing saline treated and TGFβRII/hTRβC1/IL-15:TGFβRII/IL-15RαSu complex treated B16F10 tumor-bearing mice. Arrow heads indicate treatment time.

FIG. 33C shows tumor volumes on Day 18 (end of study) between saline and TGFβRII/hTRβC1/IL-15:TGFβRII/IL-15RαSu complex group.

FIG. 34A shows frequency of total CD8⁺ T cells in peripheral blood of saline-treated and TGFβRII/hTRβC1/IL-15:TGFβRII/IL-15RαSu complex treated mice on day 3 post treatment.

FIG. 34B shows frequency of Ki67⁺ proliferating CD8⁺ T cells in peripheral blood of saline-treated and TGFβRII/hTRβC1/IL-15:TGFβRII/IL-15RαSu complex treated mice on day 3 post treatment.

FIG. 34C shows frequency of total NK1.1 cells in peripheral blood of saline-treated and TGFβRII/hTRβC1/IL-15:TGFβRII/IL-15RαSu complex treated mice on day 3 post treatment.

FIG. 34D shows frequency Ki67⁺ proliferating NK cells in peripheral blood of saline-treated and TGFβRII/hTRβC1/IL-15:TGFβRII/IL-15RαSu complex treated mice on day 3 post treatment.

FIGS. 35A and 35B show in vitro characterization of the IL-7/hTRβC1/IL-15 and IL-21/IL-15RαSu protein complex. FIG. 35A shows IL-15 activity with the HEK-Blue IL-2 assay.

FIG. 35B shows IL-7 activity with the HEK-Blue IL-7 assay. FIG. 35C shows IL-21 activity of IL-7/hTRβC1/IL-15 and IL-21/IL-15RαSu protein complex.

FIG. 36A shows a schematic of the experimental study related to FIGS. 36B-36C.

FIGS. 36B-36C are plots showing IL-7/hTRβC1/IL-15:IL-21/IL-15RαSu reduces tumor size of EG-70VA tumors and B16F10 tumors in C57BL/6 mice.

FIG. 37 shows IL-7/hTRβC1/IL-15:IL-21/IL-15RαSu protein complex activate STAT3 and STAT5 signaling pathways in human PBMCs.

FIG. 38A is an exemplary schematic of the experimental method of demonstrating a synergistic enhancement of immune activation.

FIG. 38B shows IL-7/hTRβC1/IL-15 and IL-21/IL-15RαSu protein complex significantly increased IFNγ secretion in the presence of α-PD-1 mAb.

FIG. 39A shows a schematic of the experimental study in EG7-OVA tumor bearing mice related to FIGS. 39B and 39C.

FIG. 39B shows more potent anti-tumor activities of IL-7/hTRβC1/IL-15 and IL-21/IL-15RαSu protein complex in combination with α-PD-1 mAb than monotherapy in EG7-OVA tumor bearing mice.

FIG. 39C shows the treatment of IL-7/hTRβC1/IL-15 and IL-21/IL-15RαSu protein complex in combination with α-PD-1 mAb provides better survival benefits to the tumor bearing mice than the treatment of monotherapy.

FIG. 39D shows IL-7/hTRβC1/IL-15:IL-21/IL-15RαSu in combination with α-PD-1 mAb has synergistic anti-tumor effects on EG7-OVA tumor.

FIG. 39E is a schematic of the experimental study in EG7-OVA tumor bearing mice related to FIGS. 39F.

FIG. 39F is a plot that shows IL-7/hTRβC1/IL-15:IL-21/IL-15RαSu protein complex in combination with α-PD-1 mAb established a memory immune response against EG7-OVA lymphoblast cells.

FIG. 40 is a schematic diagram of an exemplary TGFR/hTRβC1/IL-15 and αPD-1 mAb/IL-15RαSu/IL-7 fusion protein. The schematic depicts the association of the first and the second chimeric polypeptides through the pair of affinity domains.

FIG. 41 is a schematic diagram of an exemplary αTF/hTRβC1/IL-15 and αPD-1Ab/IL-15RαSu/IL-7 fusion protein. The schematic depicts the association of the first and the second chimeric polypeptides through the pair of affinity domains.

FIG. 42 is a schematic diagram of an exemplary IL-7/hTRβC1/IL-15 and αPD-1Ab/IL-15RαSu/IL-21 fusion protein. The schematic depicts the association of the first and the second chimeric polypeptides through the pair of affinity domains.

FIG. 43 shows characterization of TGFR/hTRβC1/IL-15 and αPD-1Ab/IL-15RαSu/IL-7 protein complex with size exclusion chromatography.

FIGS. 44A-44D show demonstration of the physical presence of functional domains of multi-chain chimeric polypeptides with ELISA.

FIGS. 45A-45C show biological activities of IL-15, IL-7, and TGFβRII functional domains of multi-chain chimeric polypeptides.

FIG. 46 shows binding activity of Keytruda diabody (i.e., an anti-human PD-1 antibody) of multi-chain chimeric polypeptides compared to Keytruda to hPDL-1.

FIG. 47 is a plot showing immune cell stimulatory and checkpoint blockade activity of IL-7/hTRβC1/IL-15 and αPD-1Ab/IL-15RαSu/IL-21 protein complex.

FIGS. 48A-48C show cytotoxic activity of activated human PBMCs by multi-chain chimeric polypeptides against pancreatic tumor cells.

FIG. 49 shows reduction of TGFβ31-inhibition to IFNγ production of activated human PBMCs by TGFR/hTRβC1/IL-15:αPD-1Ab/L-15RαSu/IL-7.

FIGS. 50A-50C show enhancement of granzyme R expression of activated human PBMCs by multi-chain chimeric polypeptides.

FIGS. 51A-51C show enhancement of Ki67 expression and reduction of TGFβ1-inhibition to Ki67 expression of activated human PBMCs by multi-chain chimeric polypeptides.

FIG. 52A is a schematic diagram of an exemplary IL-7/hTRβC1/IL-15 and αTFscFv/IL-15RαSu/αCD3scFv protein complex. The schematic depicts the association of the first and the second chimeric polypeptides through the pair of affinity domain.

FIG. 52B is a schematic diagram of an exemplary TGFRβRII/hTRβC1/IL-15 and αTFscFv/IL-15RαSu/αCD3scFv protein complex. The schematic depicts the association of the first and the second chimeric polypeptides through the pair of affinity domain.

FIG. 52C is a schematic diagram of an exemplary TGFβRII/hTRβC1/IL-15 and αTFscFv/IL-15RαSu/αCD16scFv protein complex. The schematic depicts the association of the first and the second chimeric polypeptides through the pair of affinity domain.

FIG. 53 is a plot showing tissue factor expression in human cancer cell lines.

FIGS. 54A-B are plots showing the quantitative flow cytometric analysis of tissue factor expression using PE beads.

FIG. 55 is a plot showing tissue factor expression across several cell lines.

FIGS. 56A and 56B show T-cell engager TGFβRII/hTRβC1/IL-15:anti-TFscFv/IL-15RαSu/anti-CD3scFv-mediated conjugation of Jurkat T cells and A431 at various concentrations.

FIG. 56C is flow cytometry analysis showing conjugation assay between SW1990 and Jurkat cells using TGFβRII/hTRβC1/IL-15:anti-TFscFv/IL-15RαSu/anti-CD3scFv.

FIG. 56D is a plot showing conjugation assay between SW1990 and Jurkat cells using TGFβRII/hTRβC1/IL-15:anti-TFscFv/IL-15RαSu/anti-CD3scFv.

FIGS. 56E-56G are plots showing ability of TGFβRII/hTRβC1/IL-15:anti-TFscFv/IL-15RαSu/anti-CD3scFv to mediate conjugate formation across various tumor lines, including A431, AsPC-1, and MIAPaCA-2 cells.

FIG. 57A is a schematic diagram of an exemplary TGFβRII/hTRβC1/IL-15 and αCD3Ab/IL-15RαSu/αTFAb fusion protein. The schematic depicts the association of the first and the second chimeric polypeptides through the pair of affinity domains.

FIG. 57B is a schematic diagram of an exemplary TGFβRII/hTRβC1/IL-15 and αTFAb/IL-15RαSu/αCD3Ab fusion protein. The schematic depicts the association of the first and the second chimeric polypeptides through the pair of affinity domains.

FIG. 57C is a schematic diagram of an exemplary IL-7/hTRβC1/IL-15 and αCD3Ab/IL-15RαSu/αTFAb fusion protein. The schematic depicts the association of the first and the second chimeric polypeptides through the pair of affinity domains.

FIG. 57D is a schematic diagram of an exemplary IL-7/hTRβC1/IL-15 and αTFAb/IL-15RαSu/αCD3Ab fusion protein. The schematic depicts the association of the first and the second chimeric polypeptides through the pair of affinity domains.

FIG. 57E is a schematic diagram of an exemplary TGFβRII/hTRβC1/IL-15 and αTFAb/IL-15RαSu/αCD3Ab fusion protein. The schematic depicts the association of the first and the second chimeric polypeptides through the pair of affinity domains.

FIG. 57F is a schematic diagram of an exemplary IL-7/hTRβC1/IL-15 and αTFAb/IL-15RαSu/αCD3Ab fusion protein. The schematic depicts the association of the first and the second chimeric polypeptides through the pair of affinity domains.

FIGS. 58A-58F show that activated T-cells release IL-2 by T-cell engagers (TCEs), wherein IL-2 released into the supernatant were measured using IL-2 immunoassay kit.

FIGS. 59A-F show evaluation of cytotoxic effects on AsPC-1 and MIA Cell lines by human PBMCs activated via T-cell engagers.

FIGS. 60A-60C are plots showing binding ability of individual component of TGFβRII/hTRβC1/IL-15:anti-TFscFv/IL-15RαSu/anti-CD3scFv and IL-7/hTRβC1/IL-15:anti-TFscFv/IL-15RαSu/anti-CD3scFv.

FIGS. 61A-61D are plots showing activity of individual component of TGFβRII/hTRβC1/IL-15:anti-TFscFv/IL-15RαSu/anti-CD3scFv and IL-7/hTRβC1/IL-15:anti-TFscFv/IL-15RαSu/anti-CD3scFv.

FIG. 62 shows evaluation of cytotoxic effects on AsPC-1 and MIA cell lines by human PBMCs activated via T-cell engagers.

FIGS. 63A and 63B show results of TCE-mediated CD8⁺ T-cell activation in human PBMCs.

FIGS. 64A and 64B show results of TCE-mediated CD4⁺ T-cell activation in human PBMCs.

FIGS. 65A-65E show cytokine profiling of T-cell engagers IL-7/hTRβC1/IL-15:anti-TFscFv/IL-15RαSu/anti-CD3scFv using CBA kit in human PBMC.

FIGS. 66A-66C show results of TCE-mediated CD8⁺ T-cell activation in CD2⁺ human PBMCs.

FIGS. 67A-67C show results of TCE-mediated CD4⁺ T-cell activation in CD2⁺ human PBMCs.

FIGS. 68A-68E show results of cytokine profiling of T-cell engagers IL-7/hTRβC1/IL-15:anti-TFscFv/IL-15RαSu/anti-CD3scFv using CBA kit in CD2⁺ human PBMC.

FIGS. 69A-69D show evaluation of IL-7/hTRβC1/IL-15:anti-TFscFv/IL-15RαSu/anti-CD3scFv and TGFβRII/hTRβC1/IL-15:anti-TFscFv/IL-15RαSu/anti-CD3scFV in a SCID mouse tumor model using AsPC-1 pancreatic tumors and human PBMCs.

FIGS. 70A and 70B show immunohistology of tumor section in TGFβRII/hTRβC1/IL-15:anti-TFscFv/IL-15RαSu/anti-CD3scFv or control treated SCID mice bearing AsPC-1 pancreatic tumors.

FIGS. 71A-71B show lymphocytes and CD3⁺ T cells infiltration in tumor by H&E and CD3⁺ T cells staining.

FIG. 72 is a schematic diagram of an exemplary TGFRβRII/hTRβC1/IL-15 and αTFscFv/IL-15RαSu/αCD16scFv protein complex. The schematic depicts the association of the first and the second chimeric polypeptides through the pair of affinity domains.

FIGS. 73A-74D are plots showing TGFβRII/hTRβC1/IL-15:anti-TFscFv/IL-15RαSu/anti-CD16a-scFv activation data in human PBMCs.

FIG. 74 is a plot showing TGFβRII/hTRβC1/IL-15:anti-TFscFv/IL-15RαSu/anti-CD16a-scFv mediated induction of CD107a in human PBMCs.

FIG. 75 is a plot showing live Raji-PDL1 cell count after incubation with a control, IL-7/hTRβC1/IL-15:IL-21/IL-15RαSu, or TGFβRII/hTRβC1/IL-15: anti-PD-1(diabody)/IL-15RαSu/IL-7 at 100 nM for 5 days.

FIG. 76A is a schematic showing a two-layer cell culture.

FIG. 76B is a plot showing live Raji-PDL1 cell count.

FIG. 77A is an image from 2 days post treatment: tumor only.

FIG. 77B is an image from 2 days post treatment: ahPBMC coculture+IL-7/hTRβC1/IL-15:IL-21/IL-15RαSu.

FIG. 77C is an image from 2 days post treatment: ahPBMC coculture+TGFβRII/hTRβC1/IL-15: anti-PD-1(diabody)/IL-15RαSu/IL-7.

FIG. 77D is an image from 2 days post treatment: tumor only.

FIG. 77E is an image from 2 days post treatment: nhPBMC coculture+IL-7/hTRβC1/IL-15:IL-21/IL-15RαSu.

FIG. 77F is an image from 2 days post treatment: nhPBMC coculture+TGFβRII/hTRβC1/IL-15: anti-PD-1(diabody)/IL-15RαSu/IL-7.

FIG. 78 is a plot showing apoptosis activity.

FIG. 79A is a plot showing H-Score for CD3 expression for saline and TGFβRII/hTRβC1/IL-15: anti-TFscFv/IL-15RαSu/anti-CD3scFv.

FIG. 79B is a representative image of immunohistochemistry staining with anti-CD3 for saline and TGFβRII/hTRβC1/IL-15: anti-TFscFv/IL-15RαSu/anti-CD3scFv. 40× magnification was used.

FIG. 79C is a plot showing H-Score for tissue factor expression for saline and TGFβRII/hTRβC1/IL-15: anti-TFscFv/IL-15RαSu/anti-CD3scFv.

FIG. 79D is a representative image of immunohistochemistry staining with anti-tissue factor for saline and TGFβRII/hTRβC1/IL-15: anti-TFscFv/IL-15RαSu/anti-CD3scFv. 40× magnification was used.

FIG. 80A shows images of lung tissue for (i) Normoxia+Vehicle (21% +Veh), (ii) Hyperoxia+Vehicle (95% +Veh), (iii) Hyperoxia+TGFβRII/hTRβC1/IL-15:TGFβRII/IL-15RαSu (95% +TGFβRII/hTRβC1/IL-15:TGFβRII/IL-15RαSu), or (iv) Hyperoxia+TGFβRII/hTRβC1/IL-15: TGFβRII/IL-15RαSu/IL-7 (95% +TGFβRII/hTRβC1/IL-15: TGFβRII/IL-15RαSu/IL-7).

FIG. 80B is a plot showing mean linear intercept (LM) scores.

### DETAILED DESCRIPTION

Provided herein are multi-chain chimeric polypeptides that include: (a) a first chimeric polypeptide including: (i) a first target-binding domain (e.g., any of the target-binding domains described herein or known in the art); (ii) a soluble T cell receptor β-chain constant region (TRβC) domain (e.g., any of the exemplary soluble TRβC domains described herein or known in the art); and (iii) a first domain of a pair of affinity domains; and (b) a second chimeric polypeptide including: (i) a second domain of a pair of affinity domains; and (ii) a second target-binding domain (e.g., any of the target-binding domains described herein or known in the art), where the first chimeric polypeptide and the second chimeric polypeptide associate through the binding of the first domain and the second domain of the pair of affinity domains. Also provided herein are compositions that include any of the multi-chain chimeric polypeptides described herein, nucleic acids that encode any of the multi-chain chimeric polypeptides described herein, and cells that include any of the nucleic acids that encode any of the multi-chain chimeric polypeptides described herein. Also provided herein are methods of stimulating an immune cell and methods of treating a subject in need thereof that include the use of any of the multi-chain chimeric polypeptides described herein. Also provided herein are methods of producing any of the multi-chain chimeric polypeptides described herein.

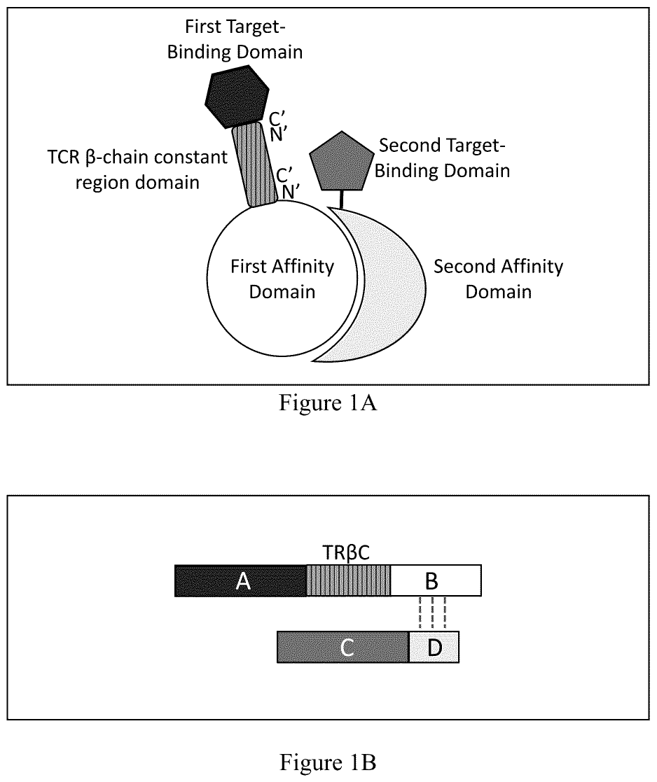

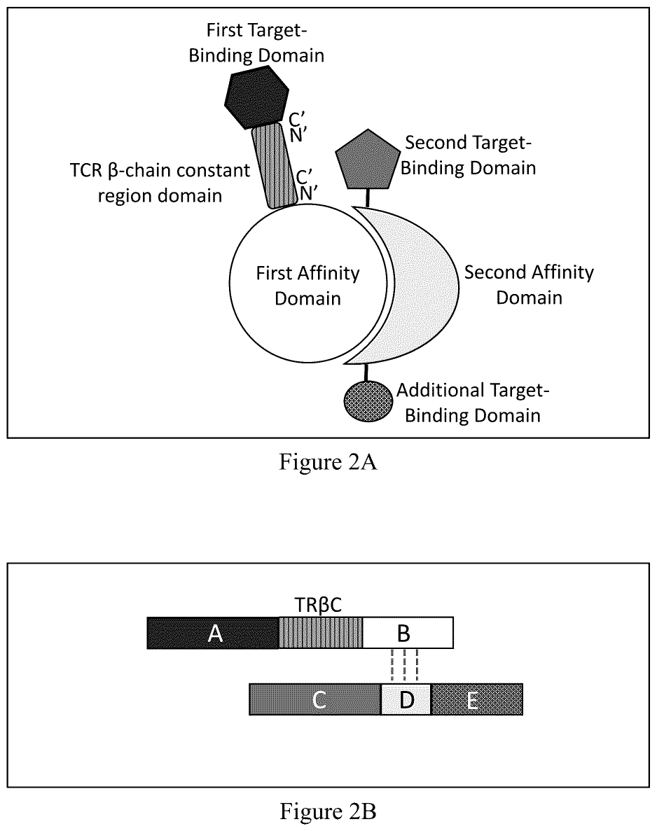

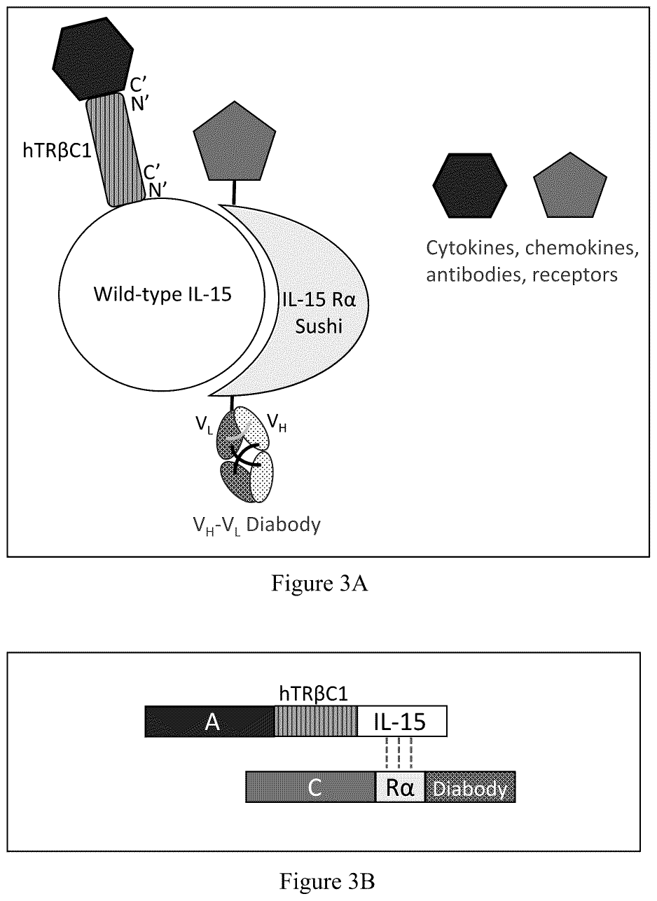

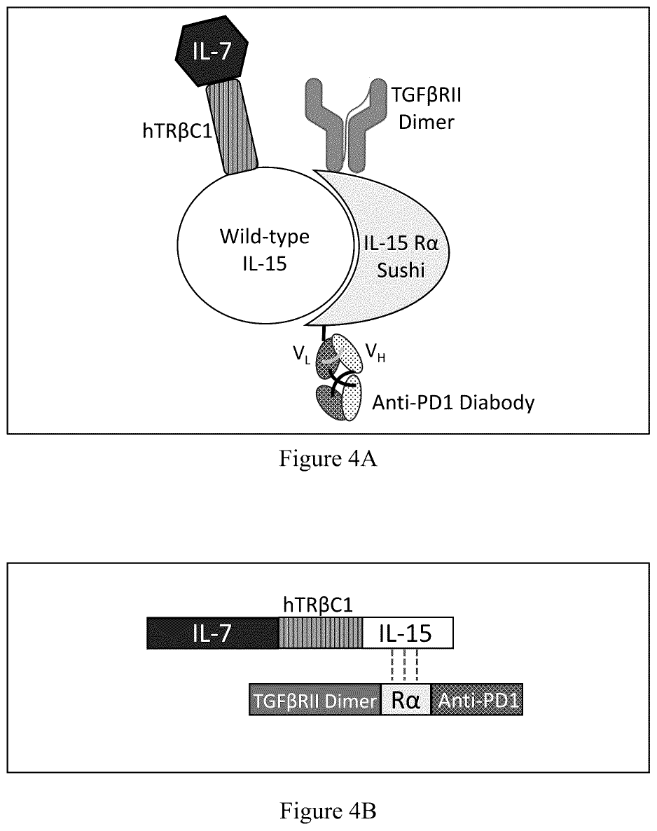

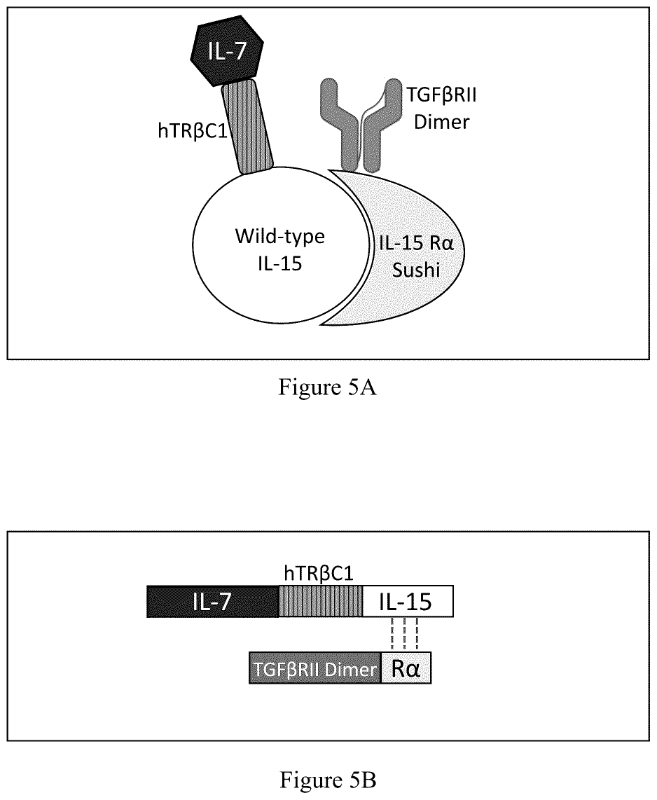

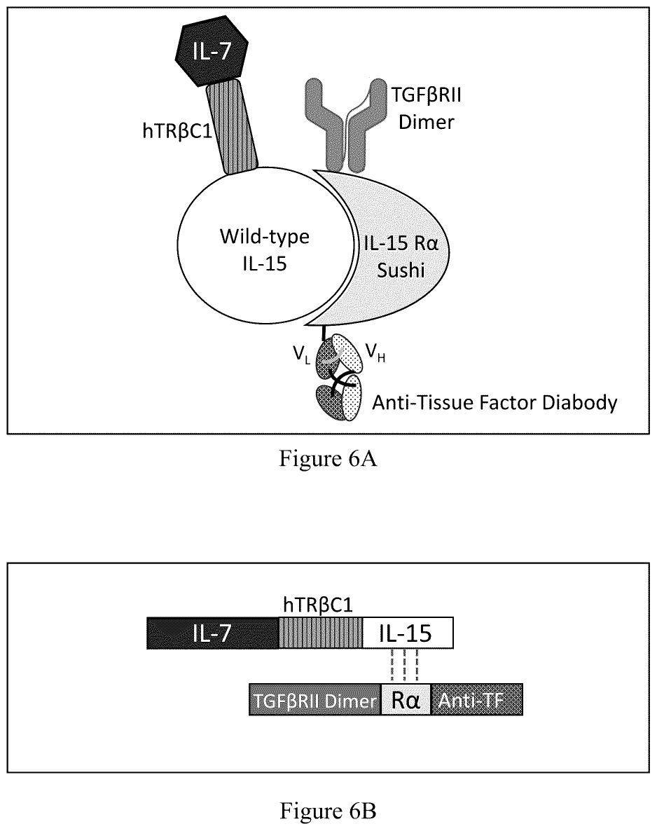

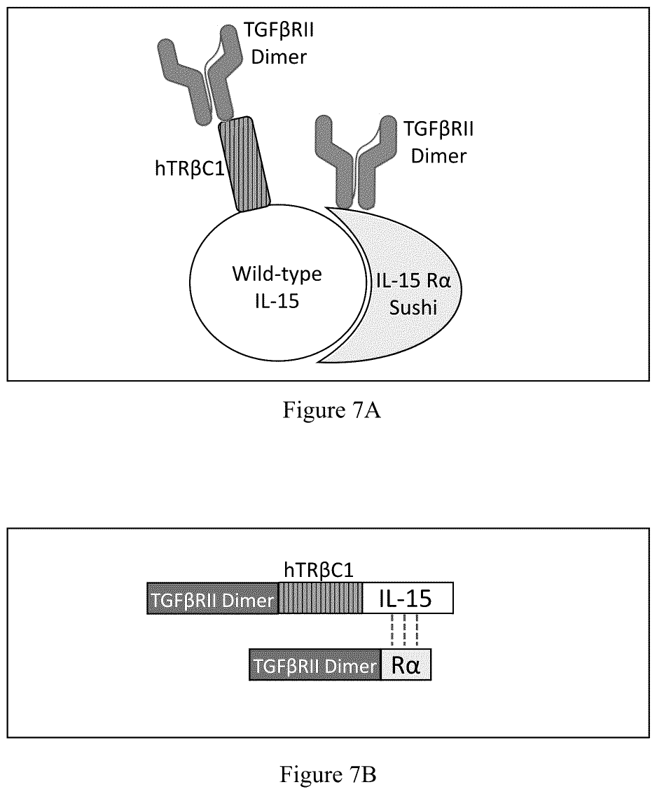

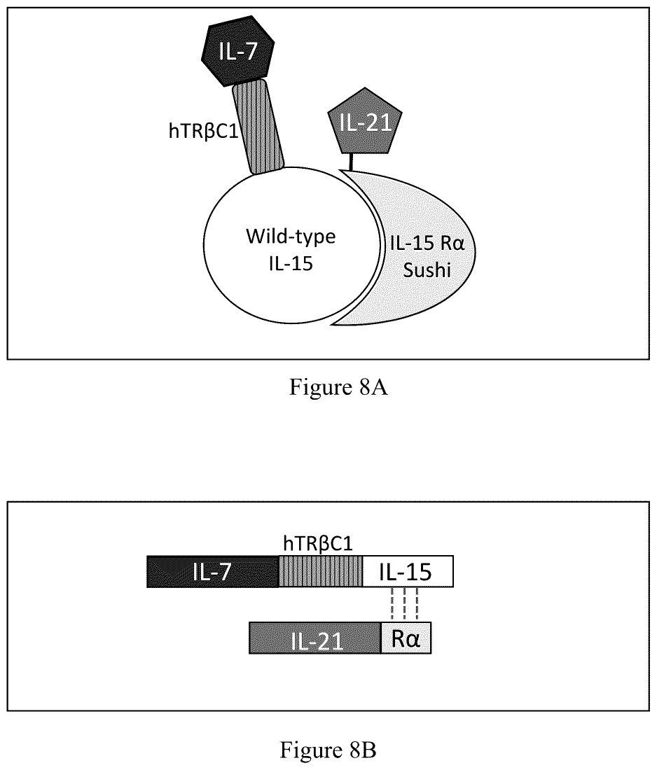

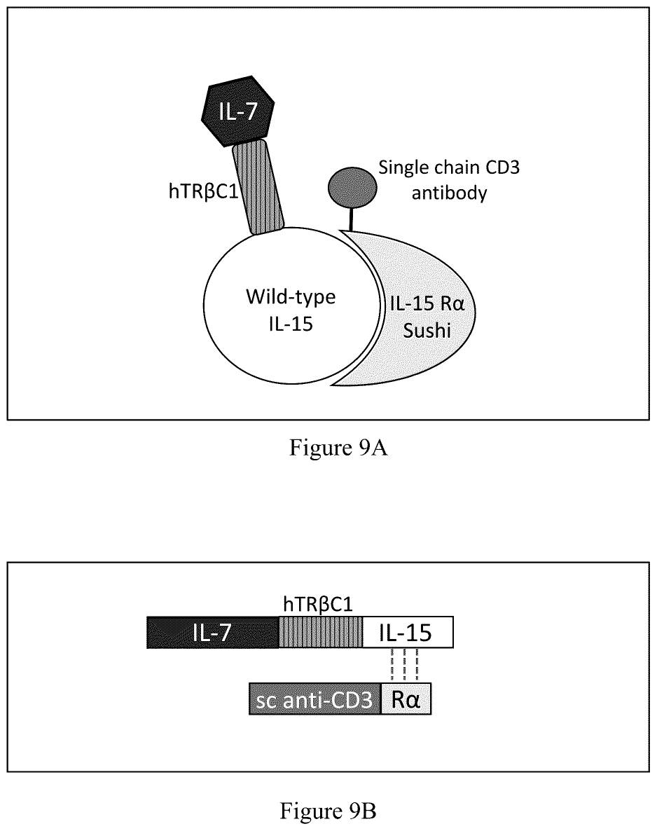

In some examples of any of the multi-chain chimeric polypeptides described herein the total length of first chimeric polypeptide and/or the second chimeric polypeptide can each independently be about 50 amino acids to about 3000 amino acids, about 50 amino acids to about 2500 amino acids, about 50 amino acids to about 2000 amino acids, about 50 amino acids to about 1500 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 480 amino acids, about 50 amino acids to about 460 amino acids, about 50 amino acids to about 440 amino acids, about 50 amino acids to about 420 amino acids, about 50 amino acids to about 400 amino acids, about 50 amino acids to about 380 amino acids, about 50 amino acids to about 360 amino acids, about 50 amino acids to about 340 amino acids, about 50 amino acids to about 320 amino acids, about 50 amino acids to about 300 amino acids, about 50 amino acids to about 280 amino acids, about 50 amino acids to about 260 amino acids, about 50 amino acids to about 240 amino acids, about 50 amino acids to about 220 amino acids, about 50 amino acids to about 200 amino acids, about 50 amino acids to about 150 amino acids, about 50 amino acids to about 100 amino acids, about 100 amino acids to about 3000 amino acids, about 100 amino acids to about 2500 amino acids, about 100 amino acids to about 2000 amino acids, about 100 amino acids to about 1500 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 480 amino acids, about 100 amino acids to about 460 amino acids, about 100 amino acids to about 440 amino acids, about 100 amino acids to about 420 amino acids, about 100 amino acids to about 400 amino acids, about 100 amino acids to about 380 amino acids, about 100 amino acids to about 360 amino acids, about 100 amino acids to about 340 amino acids, about 100 amino acids to about 320 amino acids, about 100 amino acids to about 300 amino acids, about 100 amino acids to about 280 amino acids, about 100 amino acids to about 260 amino acids, about 100 amino acids to about 240 amino acids, about 100 amino acids to about 220 amino acids, about 100 amino acids to about 200 amino acids, about 100 amino acids to about 150 amino acids, about 150 amino acids to about 3000 amino acids, about 150 amino acids to about 2500 amino acids, about 150 amino acids to about 2000 amino acids, about 150 amino acids to about 1500 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 480 amino acids, about 150 amino acids to about 460 amino acids, about 150 amino acids to about 440 amino acids, about 150 amino acids to about 420 amino acids, about 150 amino acids to about 400 amino acids, about 150 amino acids to about 380 amino acids, about 150 amino acids to about 360 amino acids, about 150 amino acids to about 340 amino acids, about 150 amino acids to about 320 amino acids, about 150 amino acids to about 300 amino acids, about 150 amino acids to about 280 amino acids, about 150 amino acids to about 260 amino acids, about 150 amino acids to about 240 amino acids, about 150 amino acids to about 220 amino acids, about 150 amino acids to about 200 amino acids, about 200 amino acids to about 3000 amino acids, about 200 amino acids to about 2500 amino acids, about 200 amino acids to about 2000 amino acids, about 200 amino acids to about 1500 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 480 amino acids, about 200 amino acids to about 460 amino acids, about 200 amino acids to about 440 amino acids, about 200 amino acids to about 420 amino acids, about 200 amino acids to about 400 amino acids, about 200 amino acids to about 380 amino acids, about 200 amino acids to about 360 amino acids, about 200 amino acids to about 340 amino acids, about 200 amino acids to about 320 amino acids, about 200 amino acids to about 300 amino acids, about 200 amino acids to about 280 amino acids, about 200 amino acids to about 260 amino acids, about 200 amino acids to about 240 amino acids, about 200 amino acids to about 220 amino acids, about 220 amino acids to about 3000 amino acids, about 220 amino acids to about 2500 amino acids, about 220 amino acids to about 2000 amino acids, about 220 amino acids to about 1500 amino acids, about 220 amino acids to about 1000 amino acids, about 220 amino acids to about 950 amino acids, about 220 amino acids to about 900 amino acids, about 220 amino acids to about 850 amino acids, about 220 amino acids to about 800 amino acids, about 220 amino acids to about 750 amino acids, about 220 amino acids to about 700 amino acids, about 220 amino acids to about 650 amino acids, about 220 amino acids to about 600 amino acids, about 220 amino acids to about 550 amino acids, about 220 amino acids to about 500 amino acids, about 220 amino acids to about 480 amino acids, about 220 amino acids to about 460 amino acids, about 220 amino acids to about 440 amino acids, about 220 amino acids to about 420 amino acids, about 220 amino acids to about 400 amino acids, about 220 amino acids to about 380 amino acids, about 220 amino acids to about 360 amino acids, about 220 amino acids to about 340 amino acids, about 220 amino acids to about 320 amino acids, about 220 amino acids to about 300 amino acids, about 220 amino acids to about 280 amino acids, about 220 amino acids to about 260 amino acids, about 220 amino acids to about 240 amino acids, about 240 amino acids to about 3000 amino acids, about 240 amino acids to about 2500 amino acids, about 240 amino acids to about 2000 amino acids, about 240 amino acids to about 1500 amino acids, about 240 amino acids to about 1000 amino acids, about 240 amino acids to about 950 amino acids, about 240 amino acids to about 900 amino acids, about 240 amino acids to about 850 amino acids, about 240 amino acids to about 800 amino acids, about 240 amino acids to about 750 amino acids, about 240 amino acids to about 700 amino acids, about 240 amino acids to about 650 amino acids, about 240 amino acids to about 600 amino acids, about 240 amino acids to about 550 amino acids, about 240 amino acids to about 500 amino acids, about 240 amino acids to about 480 amino acids, about 240 amino acids to about 460 amino acids, about 240 amino acids to about 440 amino acids, about 240 amino acids to about 420 amino acids, about 240 amino acids to about 400 amino acids, about 240 amino acids to about 380 amino acids, about 240 amino acids to about 360 amino acids, about 240 amino acids to about 340 amino acids, about 240 amino acids to about 320 amino acids, about 240 amino acids to about 300 amino acids, about 240 amino acids to about 280 amino acids, about 240 amino acids to about 260 amino acids, about 260 amino acids to about 3000 amino acids, about 260 amino acids to about 2500 amino acids, about 260 amino acids to about 2000 amino acids, about 260 amino acids to about 1500 amino acids, about 260 amino acids to about 1000 amino acids, about 260 amino acids to about 950 amino acids, about 260 amino acids to about 900 amino acids, about 260 amino acids to about 850 amino acids, about 260 amino acids to about 800 amino acids, about 260 amino acids to about 750 amino acids, about 260 amino acids to about 700 amino acids, about 260 amino acids to about 650 amino acids, about 260 amino acids to about 600 amino acids, about 260 amino acids to about 550 amino acids, about 260 amino acids to about 500 amino acids, about 260 amino acids to about 480 amino acids, about 260 amino acids to about 460 amino acids, about 260 amino acids to about 440 amino acids, about 260 amino acids to about 420 amino acids, about 260 amino acids to about 400 amino acids, about 260 amino acids to about 380 amino acids, about 260 amino acids to about 360 amino acids, about 260 amino acids to about 340 amino acids, about 260 amino acids to about 320 amino acids, about 260 amino acids to about 300 amino acids, about 260 amino acids to about 280 amino acids, about 280 amino acids to about 3000 amino acids, about 280 amino acids to about 2500 amino acids, about 280 amino acids to about 2000 amino acids, about 280 amino acids to about 1500 amino acids, about 280 amino acids to about 1000 amino acids, about 280 amino acids to about 950 amino acids, about 280 amino acids to about 900 amino acids, about 280 amino acids to about 850 amino acids, about 280 amino acids to about 800 amino acids, about 280 amino acids to about 750 amino acids, about 280 amino acids to about 700 amino acids, about 280 amino acids to about 650 amino acids, about 280 amino acids to about 600 amino acids, about 280 amino acids to about 550 amino acids, about 280 amino acids to about 500 amino acids, about 280 amino acids to about 480 amino acids, about 280 amino acids to about 460 amino acids, about 280 amino acids to about 440 amino acids, about 280 amino acids to about 420 amino acids, about 280 amino acids to about 400 amino acids, about 280 amino acids to about 380 amino acids, about 280 amino acids to about 360 amino acids, about 280 amino acids to about 340 amino acids, about 280 amino acids to about 320 amino acids, about 280 amino acids to about 300 amino acids, about 300 amino acids to about 3000 amino acids, about 300 amino acids to about 2500 amino acids, about 300 amino acids to about 2000 amino acids, about 300 amino acids to about 1500 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 480 amino acids, about 300 amino acids to about 460 amino acids, about 300 amino acids to about 440 amino acids, about 300 amino acids to about 420 amino acids, about 300 amino acids to about 400 amino acids, about 300 amino acids to about 380 amino acids, about 300 amino acids to about 360 amino acids, about 300 amino acids to about 340 amino acids, about 300 amino acids to about 320 amino acids, about 320 amino acids to about 3000 amino acids, about 320 amino acids to about 2500 amino acids, about 320 amino acids to about 2000 amino acids, about 320 amino acids to about 1500 amino acids, about 320 amino acids to about 1000 amino acids, about 320 amino acids to about 950 amino acids, about 320 amino acids to about 900 amino acids, about 320 amino acids to about 850 amino acids, about 320 amino acids to about 800 amino acids, about 320 amino acids to about 750 amino acids, about 320 amino acids to about 700 amino acids, about 320 amino acids to about 650 amino acids, about 320 amino acids to about 600 amino acids, about 320 amino acids to about 550 amino acids, about 320 amino acids to about 500 amino acids, about 320 amino acids to about 480 amino acids, about 320 amino acids to about 460 amino acids, about 320 amino acids to about 440 amino acids, about 320 amino acids to about 420 amino acids, about 320 amino acids to about 400 amino acids, about 320 amino acids to about 380 amino acids, about 320 amino acids to about 360 amino acids, about 320 amino acids to about 340 amino acids, about 340 amino acids to about 3000 amino acids, about 340 amino acids to about 2500 amino acids, about 340 amino acids to about 2000 amino acids, about 340 amino acids to about 1500 amino acids, about 340 amino acids to about 1000 amino acids, about 340 amino acids to about 950 amino acids, about 340 amino acids to about 900 amino acids, about 340 amino acids to about 850 amino acids, about 340 amino acids to about 800 amino acids, about 340 amino acids to about 750 amino acids, about 340 amino acids to about 700 amino acids, about 340 amino acids to about 650 amino acids, about 340 amino acids to about 600 amino acids, about 340 amino acids to about 550 amino acids, about 340 amino acids to about 500 amino acids, about 340 amino acids to about 480 amino acids, about 340 amino acids to about 460 amino acids, about 340 amino acids to about 440 amino acids, about 340 amino acids to about 420 amino acids, about 340 amino acids to about 400 amino acids, about 340 amino acids to about 380 amino acids, about 340 amino acids to about 360 amino acids, about 360 amino acids to about 3000 amino acids, about 360 amino acids to about 2500 amino acids, about 360 amino acids to about 2000 amino acids, about 360 amino acids to about 1500 amino acids, about 360 amino acids to about 1000 amino acids, about 360 amino acids to about 950 amino acids, about 360 amino acids to about 900 amino acids, about 360 amino acids to about 850 amino acids, about 360 amino acids to about 800 amino acids, about 360 amino acids to about 750 amino acids, about 360 amino acids to about 700 amino acids, about 360 amino acids to about 650 amino acids, about 360 amino acids to about 600 amino acids, about 360 amino acids to about 550 amino acids, about 360 amino acids to about 500 amino acids, about 360 amino acids to about 480 amino acids, about 360 amino acids to about 460 amino acids, about 360 amino acids to about 440 amino acids, about 360 amino acids to about 420 amino acids, about 360 amino acids to about 400 amino acids, about 360 amino acids to about 380 amino acids, about 380 amino acids to about 3000 amino acids, about 380 amino acids to about 2500 amino acids, about 380 amino acids to about 2000 amino acids, about 380 amino acids to about 1500 amino acids, about 380 amino acids to about 1000 amino acids, about 380 amino acids to about 950 amino acids, about 380 amino acids to about 900 amino acids, about 380 amino acids to about 850 amino acids, about 380 amino acids to about 800 amino acids, about 380 amino acids to about 750 amino acids, about 380 amino acids to about 700 amino acids, about 380 amino acids to about 650 amino acids, about 380 amino acids to about 600 amino acids, about 380 amino acids to about 550 amino acids, about 380 amino acids to about 500 amino acids, about 380 amino acids to about 480 amino acids, about 380 amino acids to about 460 amino acids, about 380 amino acids to about 440 amino acids, about 380 amino acids to about 420 amino acids, about 380 amino acids to about 400 amino acids, about 400 amino acids to about 3000 amino acids, about 400 amino acids to about 2500 amino acids, about 400 amino acids to about 2000 amino acids, about 400 amino acids to about 1500 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 480 amino acids, about 400 amino acids to about 460 amino acids, about 400 amino acids to about 440 amino acids, about 400 amino acids to about 420 amino acids, about 420 amino acids to about 3000 amino acids, about 420 amino acids to about 2500 amino acids, about 420 amino acids to about 2000 amino acids, about 420 amino acids to about 1500 amino acids, about 420 amino acids to about 1000 amino acids, about 420 amino acids to about 950 amino acids, about 420 amino acids to about 900 amino acids, about 420 amino acids to about 850 amino acids, about 420 amino acids to about 800 amino acids, about 420 amino acids to about 750 amino acids, about 420 amino acids to about 700 amino acids, about 420 amino acids to about 650 amino acids, about 420 amino acids to about 600 amino acids, about 420 amino acids to about 550 amino acids, about 420 amino acids to about 500 amino acids, about 420 amino acids to about 480 amino acids, about 420 amino acids to about 460 amino acids, about 420 amino acids to about 440 amino acids, about 440 amino acids to about 3000 amino acids, about 440 amino acids to about 2500 amino acids, about 440 amino acids to about 2000 amino acids, about 440 amino acids to about 1500 amino acids, about 440 amino acids to about 1000 amino acids, about 440 amino acids to about 950 amino acids, about 440 amino acids to about 900 amino acids, about 440 amino acids to about 850 amino acids, about 440 amino acids to about 800 amino acids, about 440 amino acids to about 750 amino acids, about 440 amino acids to about 700 amino acids, about 440 amino acids to about 650 amino acids, about 440 amino acids to about 600 amino acids, about 440 amino acids to about 550 amino acids, about 440 amino acids to about 500 amino acids, about 440 amino acids to about 480 amino acids, about 440 amino acids to about 460 amino acids, about 460 amino acids to about 3000 amino acids, about 460 amino acids to about 2500 amino acids, about 460 amino acids to about 2000 amino acids, about 460 amino acids to about 1500 amino acids, about 460 amino acids to about 1000 amino acids, about 460 amino acids to about 950 amino acids, about 460 amino acids to about 900 amino acids, about 460 amino acids to about 850 amino acids, about 460 amino acids to about 800 amino acids, about 460 amino acids to about 750 amino acids, about 460 amino acids to about 700 amino acids, about 460 amino acids to about 650 amino acids, about 460 amino acids to about 600 amino acids, about 460 amino acids to about 550 amino acids, about 460 amino acids to about 500 amino acids, about 460 amino acids to about 480 amino acids, about 480 amino acids to about 3000 amino acids, about 480 amino acids to about 2500 amino acids, about 480 amino acids to about 2000 amino acids, about 480 amino acids to about 1500 amino acids, about 480 amino acids to about 1000 amino acids, about 480 amino acids to about 950 amino acids, about 480 amino acids to about 900 amino acids, about 480 amino acids to about 850 amino acids, about 480 amino acids to about 800 amino acids, about 480 amino acids to about 750 amino acids, about 480 amino acids to about 700 amino acids, about 480 amino acids to about 650 amino acids, about 480 amino acids to about 600 amino acids, about 480 amino acids to about 550 amino acids, about 480 amino acids to about 500 amino acids, about 500 amino acids to about 3000 amino acids, about 500 amino acids to about 2500 amino acids, about 500 amino acids to about 2000 amino acids, about 500 amino acids to about 1500 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 3000 amino acids, about 550 amino acids to about 2500 amino acids, about 550 amino acids to about 2000 amino acids, about 550 amino acids to about 1500 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 3000 amino acids, about 600 amino acids to about 2500 amino acids, about 600 amino acids to about 2000 amino acids, about 600 amino acids to about 1500 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 3000 amino acids, about 650 amino acids to about 2500 amino acids, about 650 amino acids to about 2000 amino acids, about 650 amino acids to about 1500 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 3000 amino acids, about 700 amino acids to about 2500 amino acids, about 700 amino acids to about 2000 amino acids, about 700 amino acids to about 1500 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 3000 amino acids, about 750 amino acids to about 2500 amino acids, about 750 amino acids to about 2000 amino acids, about 750 amino acids to about 1500 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 3000 amino acids, about 800 amino acids to about 2500 amino acids, about 800 amino acids to about 2000 amino acids, about 800 amino acids to about 1500 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 3000 amino acids, about 850 amino acids to about 2500 amino acids, about 850 amino acids to about 2000 amino acids, about 850 amino acids to about 1500 amino acids, about 850 amino acids to about 1000 amino acids, about 850 amino acids to about 950 amino acids, about 850 amino acids to about 900 amino acids, about 900 amino acids to about 3000 amino acids, about 900 amino acids to about 2500 amino acids, about 900 amino acids to about 2000 amino acids, about 900 amino acids to about 1500 amino acids, about 900 amino acids to about 1000 amino acids, about 900 amino acids to about 950 amino acids, about 950 amino acids to about 3000 amino acids, about 950 amino acids to about 2500 amino acids, about 950 amino acids to about 2000 amino acids, about 950 amino acids to about 1500 amino acids, about 950 amino acids to about 1000 amino acids, about 1000 amino acids to about 3000 amino acids, about 1000 amino acids to about 2500 amino acids, about 1000 amino acids to about 2000 amino acids, about 1000 amino acids to about 1500 amino acids, about 1500 amino acids to about 3000 amino acids, about 1500 amino acids to about 2500 amino acids, about 1500 amino acids to about 2000 amino acids, about 2000 amino acids to about 3000 amino acids, about 2000 amino acids to about 2500 amino acids, or about 2500 amino acids to about 3000 amino acids. Diagrams of exemplary multi-chain chimeric polypeptides provided herein are depicted in FIGS. 1A-1B and 2A-2B.

In some embodiments of any of the multi-chain chimeric polypeptides described herein, the first target-binding domain (e.g., any of the first target-binding domains described herein) and the soluble TRβC domain (e.g., any of the exemplary soluble TRβC domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the first target-binding domain (e.g., any of the exemplary first target-binding domains described herein) and the soluble TRβC domain (e.g., any of the exemplary soluble TRβC domains described herein) in the first chimeric polypeptide.

In some embodiments of any of the multi-chain chimeric polypeptides described herein, the soluble TRβC domain (e.g., any of the exemplary soluble TRβC domains described herein) and the first domain of the pair of affinity domains (e.g., any of the exemplary first domains of any of the exemplary pairs of affinity domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the soluble TRβC domain (e.g., any of the exemplary soluble TRβC domains described herein) and the first domain of the pair of affinity domains (e.g., any of the exemplary first domains of any of the exemplary pairs of affinity domains described herein) in the first chimeric polypeptide.

In some embodiments of any of the multi-chain chimeric polypeptides described herein, the second domain of the pair of affinity domains (e.g., any of the exemplary second domains of any of the exemplary pairs of affinity domains described herein) and the second target-binding domain (e.g., any of the exemplary second target-binding domains described herein) directly abut each other in the second chimeric polypeptide.

In some embodiments of any of the multi-chain chimeric polypeptides described herein, the second chimeric polypeptide further comprises a linker sequence (e.g., any of the exemplary linker sequences described herein or known in the art) between the second domain of the pair of affinity domains (e.g., any of the exemplary second domains of any of the exemplary pairs of affinity domains described herein) and the second target-binding domain (e.g., any of the exemplary second target-binding domains described herein) in the second chimeric polypeptide.

Non-limiting aspects of these chimeric polypeptides, nucleic acids, vectors, cells, and methods are described below, and can be used in any combination without limitation. Additional aspects of these chimeric polypeptides, nucleic acids, vectors, cells, and methods are known in the art.

### T Cell Receptor β-Chain Constant Region (TRβC)

The T-cell receptor (TCR) is a heterodimer of two chains, an α-chain and a β-chain or a γ-chain and a δ-chain. An individual T cell can express either an αβ heterodimer or a γδ heterodimer as its receptor. The TCR is expressed with cluster of differentiation 3 (CD3) polypeptides and forms a complex comprising the TCR and six CD3 polypeptides (the TCR-CD3 complex). Three dimers of CD3 proteins, δε and γε heterodimers and ζζ homodimers, are present in the TCR-CD3 complex. TCR-CD3 complexes can be found on the surface of T cells where they recognize self or foreign antigens presented by major histocompatibility complex (MHC) molecules and can be involved in immune synapse formation, inducing intracellular signaling, and initiating target cell killing. The TCR is involved in antigen recognition, and the CD3 polypeptides contain intracellular signaling domains that couple the TCR-CD3 complex to the downstream signaling machinery.

TCR chains include an extracellular region, a transmembrane region, and a shorter cytoplasmic tail. The extracellular regions of TCR chains contain a variable (V) region and a constant (C) region. TCR diversity can be generated by somatic V(D)J recombination in which variable (V) regions and joining (J) regions or variable (V) regions, joining (J) regions, and diversity (D) regions can be combined during the production of a TCR chain (e.g., V, D and J for β and δ chains; V and J for α and γ chains). Cells in a clonal T cell population express the same unique TCR.

Different species have 1 to 5 different genes encoding the T cell receptor β-chain constant region (TRBC or TRβC) domain. Human and mice have genes encoding the TRβC1 (TRBC1 gene) or TRβC2 (TRBC2 gene) isoforms. Human TRβC1 and TRβC2 differ by only four amino acid mutations in the extracellular domain. The surrounding sequence is identical between the two isoforms and the folded structure remains largely unchanged. Each TCR (and therefore each T cell) selects a TCR β-chain constant region encoded by either TRβC1 or TRβC2 for expression in a mutually exclusive manner. Hence, a population of normal T cells will comprise a mixture of individual cells, some expressing TRβC1 and others expressing TRβC2, whereas the entire population of a T cell cancer will express either TRβC1 or TRβC2.

### Soluble T Cell Receptor β-Chain Constant Region (TRβC) Domains

In some embodiments of any of the multi-chain chimeric polypeptides, compositions, or methods described herein, the soluble TRβC domain can be a soluble human TRβC domain. In some embodiments, the soluble TRβC domain can be a soluble human TRβC1 domain. In some embodiments, the soluble TRβC domain can be a soluble human TRβC2 domain. Non-limiting examples of soluble TRβC domains are shown below.

In some embodiments, a soluble T cell receptor β-chain constant region (TRC) domain can comprise or consist of a sequence that is at least 80%, 85%, 90%, 92%, 95%, 96%, 98%, or 100% identical to SEQ ID NO: 1 or 3. In some embodiments, a soluble T cell receptor β-chain constant region (TRC) domain is encoded by a nucleic acid that is at least 80%, 85%, 90%, 92%, 95%, 96%, 98%, or 100% identical to SEQ ID NO: 2.

Exemplary Soluble Human TRβC1 Domain
(SEQ ID NO: 1)
EDLNKVFPPEVAVFEPSEAEISHTQKATLVCLATGFFPDHVELSWWV
NGKEVHSGVSTDPQPLKEQPALNDSRYCLSSRLRVSATFWQNPRNHE
RCQVQFYGLSENDEWTQDRAKPVTQIVSAEAWGRAD
Exemplary Nucleic Acid Encoding Soluble Human
TRβC1 Domain
(SEQ ID NO: 2)
GAGGACCTGAACAAGGTGTTCCCACCCGAGGTCGCTGTGTTTGAGCC
ATCAGAAGCAGAGATCTCCCACACCCAAAAGGCCACACTGGTGTGCC
TGGCCACAGGCTTCTTCCCCGACCACGTGGAGCTGAGCTGGTGGGTG
AATGGGAAGGAGGTGCACAGTGGGGTCAGCACGGACCCGCAGCCCCT
CAAGGAGCAGCCCGCCCTCAATGACTCCAGATACTGCCTGAGCAGCC
GCCTGAGGGTCTCGGCCACCTTCTGGCAGAACCCCCGCAACCACTTC
CGCTGTCAAGTCCAGTTCTACGGGCTCTCGGAGAATGACGAGTGGAC
CCAGGATAGGGCCAAACCCGTCACCCAGATCGTCAGCGCCGAGGCCT
GGGGTAGAGCAGAC
Exemplary Soluble Human TRβC2 Domain
(SEQ ID NO: 3)
EDLKNVFPPEVAVFEPSEAEISHTQKATLVCLATGFYPDHVELSWWV
NGKEVHSGVSTDPQPLKEQPALNDSRYCLSSRLRVSATFWQNPRNHE
RCQVQFYGLSENDEWTQDRAKPVTQIVSAEAWGRAD

As can be appreciated in the art, one skilled in the art would understand that mutation of amino acids that are conserved between different mammalian species is more likely to decrease the activity and/or structural stability of the protein, while mutation of amino acids that are not conserved between different mammalian species is less likely to decrease the activity and/or structural stability of the protein.

In some embodiments, the soluble TRβC domain can comprise or consist of a soluble wildtype human TRβC2 domain (or any sequence therefrom).

In some embodiments, the soluble TRβC domain does not bind to an MHC protein with or without a loaded antigen. In some embodiments, the soluble TRβC domain does not stimulate an immune response in a mammal.

In some embodiments, the soluble TRβC domain can have a total length of about 20 amino acids to about 200 amino acids, about 20 amino acids to about 195 amino acids, about 20 amino acids to about 190 amino acids, about 20 amino acids to about 185 amino acids, about 20 amino acids to about 180 amino acids, about 20 amino acids to about 175 amino acids, about 20 amino acids to about 170 amino acids, about 20 amino acids to about 165 amino acids, about 20 amino acids to about 160 amino acids, acids, about 20 amino acids to about 155 amino acids, about 20 amino acids to about 150 amino acids, about 20 amino acids to about 145 amino acids, about 20 amino acids to about 140 amino acids, about 20 amino acids to about 135 amino acids, about 20 amino acids to about 130 amino acids, about 20 amino acids to about 125 amino acids, about 20 amino acids to about 120 amino acids, about 20 amino acids to about 115 amino acids, about 20 amino acids to about 110 amino acids, about 20 amino acids to about 105 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 60 amino acids, about 20 amino acids to about 50 amino acids, about 20 amino acids to about 40 amino acids, about 20 amino acids to about 30 amino acids, about 30 amino acids to about 200 amino acids, about 30 amino acids to about 195 amino acids, about 30 amino acids to about 190 amino acids, about 30 amino acids to about 185 amino acids, about 30 amino acids to about 180 amino acids, about 30 amino acids to about 175 amino acids, about 30 amino acids to about 170 amino acids, about 30 amino acids to about 165 amino acids, about 30 amino acids to about 160 amino acids, acids, about 30 amino acids to about 155 amino acids, about 30 amino acids to about 150 amino acids, about 30 amino acids to about 145 amino acids, about 30 amino acids to about 140 amino acids, about 30 amino acids to about 135 amino acids, about 30 amino acids to about 130 amino acids, about 30 amino acids to about 125 amino acids, about 30 amino acids to about 120 amino acids, about 30 amino acids to about 115 amino acids, about 30 amino acids to about 110 amino acids, about 30 amino acids to about 105 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 60 amino acids, about 30 amino acids to about 50 amino acids, about 30 amino acids to about 40 amino acids, about 40 amino acids to about 200 amino acids, about 40 amino acids to about 195 amino acids, about 40 amino acids to about 190 amino acids, about 40 amino acids to about 185 amino acids, about 40 amino acids to about 180 amino acids, about 40 amino acids to about 175 amino acids, about 40 amino acids to about 170 amino acids, about 40 amino acids to about 165 amino acids, about 40 amino acids to about 160 amino acids, acids, about 40 amino acids to about 155 amino acids, about 40 amino acids to about 150 amino acids, about 40 amino acids to about 145 amino acids, about 40 amino acids to about 140 amino acids, about 40 amino acids to about 135 amino acids, about 40 amino acids to about 130 amino acids, about 40 amino acids to about 125 amino acids, about 40 amino acids to about 120 amino acids, about 40 amino acids to about 115 amino acids, about 40 amino acids to about 110 amino acids, about 40 amino acids to about 105 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 60 amino acids, about 40 amino acids to about 50 amino acids, about 50 amino acids to about 200 amino acids, about 50 amino acids to about 195 amino acids, about 50 amino acids to about 190 amino acids, about 50 amino acids to about 185 amino acids, about 50 amino acids to about 180 amino acids, about 50 amino acids to about 175 amino acids, about 50 amino acids to about 170 amino acids, about 50 amino acids to about 165 amino acids, about 50 amino acids to about 160 amino acids, acids, about 50 amino acids to about 155 amino acids, about 50 amino acids to about 150 amino acids, about 50 amino acids to about 145 amino acids, about 50 amino acids to about 140 amino acids, about 50 amino acids to about 135 amino acids, about 50 amino acids to about 130 amino acids, about 50 amino acids to about 125 amino acids, about 50 amino acids to about 120 amino acids, about 50 amino acids to about 115 amino acids, about 50 amino acids to about 110 amino acids, about 50 amino acids to about 105 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 60 amino acids, about 60 amino acids to about 200 amino acids, about 60 amino acids to about 195 amino acids, about 60 amino acids to about 190 amino acids, about 60 amino acids to about 185 amino acids, about 60 amino acids to about 180 amino acids, about 60 amino acids to about 175 amino acids, about 60 amino acids to about 170 amino acids, about 60 amino acids to about 165 amino acids, about 60 amino acids to about 160 amino acids, acids, about 60 amino acids to about 155 amino acids, about 60 amino acids to about 150 amino acids, about 60 amino acids to about 145 amino acids, about 60 amino acids to about 140 amino acids, about 60 amino acids to about 135 amino acids, about 60 amino acids to about 130 amino acids, about 60 amino acids to about 125 amino acids, about 60 amino acids to about 120 amino acids, about 60 amino acids to about 115 amino acids, about 60 amino acids to about 110 amino acids, about 60 amino acids to about 105 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 70 amino acids to about 200 amino acids, about 70 amino acids to about 195 amino acids, about 70 amino acids to about 190 amino acids, about 70 amino acids to about 185 amino acids, about 70 amino acids to about 180 amino acids, about 70 amino acids to about 175 amino acids, about 70 amino acids to about 170 amino acids, about 70 amino acids to about 165 amino acids, about 70 amino acids to about 160 amino acids, acids, about 70 amino acids to about 155 amino acids, about 70 amino acids to about 150 amino acids, about 70 amino acids to about 145 amino acids, about 70 amino acids to about 140 amino acids, about 70 amino acids to about 135 amino acids, about 70 amino acids to about 130 amino acids, about 70 amino acids to about 125 amino acids, about 70 amino acids to about 120 amino acids, about 70 amino acids to about 115 amino acids, about 70 amino acids to about 110 amino acids, about 70 amino acids to about 105 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 80 amino acids to about 200 amino acids, about 80 amino acids to about 195 amino acids, about 80 amino acids to about 190 amino acids, about 80 amino acids to about 185 amino acids, about 80 amino acids to about 180 amino acids, about 80 amino acids to about 175 amino acids, about 80 amino acids to about 170 amino acids, about 80 amino acids to about 165 amino acids, about 80 amino acids to about 160 amino acids, acids, about 80 amino acids to about 155 amino acids, about 80 amino acids to about 150 amino acids, about 80 amino acids to about 145 amino acids, about 80 amino acids to about 140 amino acids, about 80 amino acids to about 135 amino acids, about 80 amino acids to about 130 amino acids, about 80 amino acids to about 125 amino acids, about 80 amino acids to about 120 amino acids, about 80 amino acids to about 115 amino acids, about 80 amino acids to about 110 amino acids, about 80 amino acids to about 105 amino acids, about 80 amino acids to about 100 amino acids, about 80 amino acids to about 95 amino acids, about 80 amino acids to about 90 amino acids, about 90 amino acids to about 200 amino acids, about 90 amino acids to about 195 amino acids, about 90 amino acids to about 190 amino acids, about 90 amino acids to about 185 amino acids, about 90 amino acids to about 180 amino acids, about 90 amino acids to about 175 amino acids, about 90 amino acids to about 170 amino acids, about 90 amino acids to about 165 amino acids, about 90 amino acids to about 160 amino acids, acids, about 90 amino acids to about 155 amino acids, about 90 amino acids to about 150 amino acids, about 90 amino acids to about 145 amino acids, about 90 amino acids to about 140 amino acids, about 90 amino acids to about 135 amino acids, about 90 amino acids to about 130 amino acids, about 90 amino acids to about 125 amino acids, about 90 amino acids to about 120 amino acids, about 90 amino acids to about 115 amino acids, about 90 amino acids to about 110 amino acids, about 90 amino acids to about 105 amino acids, about 90 amino acids to about 100 amino acids, about 100 amino acids to about 200 amino acids, about 100 amino acids to about 195 amino acids, about 100 amino acids to about 190 amino acids, about 100 amino acids to about 185 amino acids, about 100 amino acids to about 180 amino acids, about 100 amino acids to about 175 amino acids, about 100 amino acids to about 170 amino acids, about 100 amino acids to about 165 amino acids, about 100 amino acids to about 160 amino acids, acids, about 100 amino acids to about 155 amino acids, about 100 amino acids to about 150 amino acids, about 100 amino acids to about 145 amino acids, about 100 amino acids to about 140 amino acids, about 100 amino acids to about 135 amino acids, about 100 amino acids to about 130 amino acids, about 100 amino acids to about 125 amino acids, about 100 amino acids to about 120 amino acids, about 100 amino acids to about 115 amino acids, about 100 amino acids to about 110 amino acids, about 110 amino acids to about 200 amino acids, about 110 amino acids to about 195 amino acids, about 110 amino acids to about 190 amino acids, about 110 amino acids to about 185 amino acids, about 110 amino acids to about 180 amino acids, about 110 amino acids to about 175 amino acids, about 110 amino acids to about 170 amino acids, about 110 amino acids to about 165 amino acids, about 110 amino acids to about 160 amino acids, acids, about 110 amino acids to about 155 amino acids, about 110 amino acids to about 150 amino acids, about 110 amino acids to about 145 amino acids, about 110 amino acids to about 140 amino acids, about 110 amino acids to about 135 amino acids, about 110 amino acids to about 130 amino acids, about 110 amino acids to about 125 amino acids, about 110 amino acids to about 120 amino acids, about 110 amino acids to about 115 amino acids, about 115 amino acids to about 200 amino acids, about 115 amino acids to about 195 amino acids, about 115 amino acids to about 190 amino acids, about 115 amino acids to about 185 amino acids, about 115 amino acids to about 180 amino acids, about 115 amino acids to about 175 amino acids, about 115 amino acids to about 170 amino acids, about 115 amino acids to about 165 amino acids, about 115 amino acids to about 160 amino acids, acids, about 115 amino acids to about 155 amino acids, about 115 amino acids to about 150 amino acids, about 115 amino acids to about 145 amino acids, about 115 amino acids to about 140 amino acids, about 115 amino acids to about 135 amino acids, about 115 amino acids to about 130 amino acids, about 115 amino acids to about 125 amino acids, about 115 amino acids to about 120 amino acids, about 120 amino acids to about 200 amino acids, about 120 amino acids to about 195 amino acids, about 120 amino acids to about 190 amino acids, about 120 amino acids to about 185 amino acids, about 120 amino acids to about 180 amino acids, about 120 amino acids to about 175 amino acids, about 120 amino acids to about 170 amino acids, about 120 amino acids to about 165 amino acids, about 120 amino acids to about 160 amino acids, acids, about 120 amino acids to about 155 amino acids, about 120 amino acids to about 150 amino acids, about 120 amino acids to about 145 amino acids, about 120 amino acids to about 140 amino acids, about 120 amino acids to about 135 amino acids, about 120 amino acids to about 130 amino acids, about 120 amino acids to about 125 amino acids, about 125 amino acids to about 200 amino acids, about 125 amino acids to about 195 amino acids, about 125 amino acids to about 190 amino acids, about 125 amino acids to about 185 amino acids, about 125 amino acids to about 180 amino acids, about 125 amino acids to about 175 amino acids, about 125 amino acids to about 170 amino acids, about 125 amino acids to about 165 amino acids, about 125 amino acids to about 160 amino acids, acids, about 125 amino acids to about 155 amino acids, about 125 amino acids to about 150 amino acids, about 125 amino acids to about 145 amino acids, about 125 amino acids to about 140 amino acids, about 125 amino acids to about 135 amino acids, about 125 amino acids to about 130 amino acids, about 130 amino acids to about 200 amino acids, about 130 amino acids to about 195 amino acids, about 130 amino acids to about 190 amino acids, about 130 amino acids to about 185 amino acids, about 130 amino acids to about 180 amino acids, about 130 amino acids to about 175 amino acids, about 130 amino acids to about 170 amino acids, about 130 amino acids to about 165 amino acids, about 130 amino acids to about 160 amino acids, acids, about 130 amino acids to about 155 amino acids, about 130 amino acids to about 150 amino acids, about 130 amino acids to about 145 amino acids, about 130 amino acids to about 140 amino acids, about 130 amino acids to about 135 amino acids, about 135 amino acids to about 200 amino acids, about 135 amino acids to about 195 amino acids, about 135 amino acids to about 190 amino acids, about 135 amino acids to about 185 amino acids, about 135 amino acids to about 180 amino acids, about 135 amino acids to about 175 amino acids, about 135 amino acids to about 170 amino acids, about 135 amino acids to about 165 amino acids, about 135 amino acids to about 160 amino acids, acids, about 135 amino acids to about 155 amino acids, about 135 amino acids to about 150 amino acids, about 135 amino acids to about 145 amino acids, about 135 amino acids to about 140 amino acids, about 140 amino acids to about 200 amino acids, about 140 amino acids to about 195 amino acids, about 140 amino acids to about 190 amino acids, about 140 amino acids to about 185 amino acids, about 140 amino acids to about 180 amino acids, about 140 amino acids to about 175 amino acids, about 140 amino acids to about 170 amino acids, about 140 amino acids to about 165 amino acids, about 140 amino acids to about 160 amino acids, acids, about 140 amino acids to about 155 amino acids, about 140 amino acids to about 150 amino acids, about 140 amino acids to about 145 amino acids, about 145 amino acids to about 200 amino acids, about 145 amino acids to about 195 amino acids, about 145 amino acids to about 190 amino acids, about 145 amino acids to about 185 amino acids, about 145 amino acids to about 180 amino acids, about 145 amino acids to about 175 amino acids, about 145 amino acids to about 170 amino acids, about 145 amino acids to about 165 amino acids, about 145 amino acids to about 160 amino acids, acids, about 145 amino acids to about 155 amino acids, about 145 amino acids to about 150 amino acids, about 150 amino acids to about 200 amino acids, about 150 amino acids to about 195 amino acids, about 150 amino acids to about 190 amino acids, about 150 amino acids to about 185 amino acids, about 150 amino acids to about 180 amino acids, about 150 amino acids to about 175 amino acids, about 150 amino acids to about 170 amino acids, about 150 amino acids to about 165 amino acids, about 150 amino acids to about 160 amino acids, acids, about 150 amino acids to about 155 amino acids, about 155 amino acids to about 200 amino acids, about 155 amino acids to about 195 amino acids, about 155 amino acids to about 190 amino acids, about 155 amino acids to about 185 amino acids, about 155 amino acids to about 180 amino acids, about 155 amino acids to about 175 amino acids, about 155 amino acids to about 170 amino acids, about 155 amino acids to about 165 amino acids, about 155 amino acids to about 160 amino acids, acids, about 160 amino acids to about 200 amino acids, about 160 amino acids to about 195 amino acids, about 160 amino acids to about 190 amino acids, about 160 amino acids to about 185 amino acids, about 160 amino acids to about 180 amino acids, about 160 amino acids to about 175 amino acids, about 160 amino acids to about 170 amino acids, about 160 amino acids to about 165 amino acids, about 165 amino acids to about 200 amino acids, about 165 amino acids to about 195 amino acids, about 165 amino acids to about 190 amino acids, about 165 amino acids to about 185 amino acids, about 165 amino acids to about 180 amino acids, about 165 amino acids to about 175 amino acids, about 165 amino acids to about 170 amino acids, about 170 amino acids to about 200 amino acids, about 170 amino acids to about 195 amino acids, about 170 amino acids to about 190 amino acids, about 170 amino acids to about 185 amino acids, about 170 amino acids to about 180 amino acids, about 170 amino acids to about 175 amino acids, about 175 amino acids to about 200 amino acids, about 175 amino acids to about 195 amino acids, about 175 amino acids to about 190 amino acids, about 175 amino acids to about 185 amino acids, about 175 amino acids to about 180 amino acids, about 180 amino acids to about 200 amino acids, about 180 amino acids to about 195 amino acids, about 180 amino acids to about 190 amino acids, about 180 amino acids to about 185 amino acids, about 185 amino acids to about 200 amino acids, about 185 amino acids to about 195 amino acids, about 185 amino acids to about 190 amino acids, about 190 amino acids to about 200 amino acids, about 190 amino acids to about 195 amino acids, or about 195 amino acids to about 200 amino acids.

### Target-Binding Domains

In some embodiments of any of the multi-chain chimeric polypeptides described herein, the first target-binding domain, the second target-binding domain, and/or the one or more additional target-binding domains can be an antigen-binding domain (e.g., any of the exemplary antigen-binding domains described herein or known in the art), a soluble interleukin or cytokine protein (e.g., any of the exemplary soluble interleukin proteins or soluble cytokine proteins described herein), and a soluble interleukin or cytokine receptor (e.g., any of the exemplary soluble interleukin receptors or soluble cytokine receptors described herein).

In some embodiments of any of the multi-chain chimeric polypeptides described herein, one or more of the first target-binding domain (e.g., any of the exemplary first target-binding domains described herein or known in the art), the second target-binding domain (e.g., any of the exemplary second target-binding domains described herein or known in the art), and the one or more additional target-binding domains can each, independently, bind specifically to a target selected from the group of: bind specifically to a target selected from the group consisting of: DDL-3, GPC3, CD73, CECAM5/6, tissue factor (CD142), CD16a, CD28, CD3 (e.g., one or more of CD3a, CD3P, CD36, CD38, and CD37), CD33, CD20, CD19, CD22, CD123, IL-1R, IL-1, VEGF, IL-6R, IL-4, IL-10, PDL-1, TIGIT, PD-1, TIM3, CTLA4, MICA, MICB, IL-6, IL-8, TNFα, CD26a, CD36, ULBP2, CD30, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5/6, uPAR (urokinase plasminogen activator receptor), a UL16-binding protein (e.g., ULBP1, ULBP2, ULBP3, ULBP4, ULBP5, and ULBP6), HLA-DR, DLL4, TYRO3, AXL, MER, CD122, CD155, PDGF-DD, a ligand of TGF-β receptor II (TGF-βRII), a ligand of TGF-βRIII, a ligand of DNAM1, a ligand of NKp46, a ligand of NKp44, a ligand of NKG2D, a ligand of NKp30, a ligand for a scMHCI, a ligand for a scMHCII, a ligand for a scTCR, a ligand of ICOS, a receptor for IL-1, a receptor for IL-2, a receptor for IL-3, a receptor for IL-4, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-DD, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28.

In some embodiments of any of the multi-chain chimeric polypeptides described herein, the first target-binding domain, the second target-binding domain, and/or the one or more additional target-binding domains can each independently have a total number of amino acids of about 5 amino acids to about 1000 amino acids, about 5 amino acids to about 950 amino acids, about 5 amino acids to about 900 amino acids, about 5 amino acids to about 850 amino acids, about 5 amino acids to about 800 amino acids, about 5 amino acids to about 750 amino acids, about 5 amino acids to about 700 amino acids, about 5 amino acids to about 650 amino acids, about 5 amino acids to about 600 amino acids, about 5 amino acids to about 550 amino acids, about 5 amino acids to about 500 amino acids, about 5 amino acids to about 450 amino acids, about 5 amino acids to about 400 amino acids, about 5 amino acids to about 350 amino acids, about 5 amino acids to about 300 amino acids, about 5 amino acids to about 280 amino acids, about 5 amino acids to about 260 amino acids, about 5 amino acids to about 240 amino acids, about 5 amino acids to about 220 amino acids, about 5 amino acids to about 200 amino acids, about 5 amino acids to about 195 amino acids, about 5 amino acids to about 190 amino acids, about 5 amino acids to about 185 amino acids, about 5 amino acids to about 180 amino acids, about 5 amino acids to about 175 amino acids, about 5 amino acids to about 170 amino acids, about 5 amino acids to about 165 amino acids, about 5 amino acids to about 160 amino acids, about 5 amino acids to about 155 amino acids, about 5 amino acids to about 150 amino acids, about 5 amino acids to about 145 amino acids, about 5 amino acids to about 140 amino acids, about 5 amino acids to about 135 amino acids, about 5 amino acids to about 130 amino acids, about 5 amino acids to about 125 amino acids, about 5 amino acids to about 120 amino acids, about 5 amino acids to about 115 amino acids, about 5 amino acids to about 110 amino acids, about 5 amino acids to about 105 amino acids, about 5 amino acids to about 100 amino acids, about 5 amino acids to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 1000 amino acids, about 10 amino acids to about 950 amino acids, about 10 amino acids to about 900 amino acids, about 10 amino acids to about 850 amino acids, about 10 amino acids to about 800 amino acids, about 10 amino acids to about 750 amino acids, about 10 amino acids to about 700 amino acids, about 10 amino acids to about 650 amino acids, about 10 amino acids to about 600 amino acids, about 10 amino acids to about 550 amino acids, about 10 amino acids to about 500 amino acids, about 10 amino acids to about 450 amino acids, about 10 amino acids to about 400 amino acids, about 10 amino acids to about 350 amino acids, about 10 amino acids to about 300 amino acids, about 10 amino acids to about 280 amino acids, about 10 amino acids to about 260 amino acids, about 10 amino acids to about 240 amino acids, about 10 amino acids to about 220 amino acids, about 10 amino acids to about 200 amino acids, about 10 amino acids to about 195 amino acids, about 10 amino acids to about 190 amino acids, about 10 amino acids to about 185 amino acids, about 10 amino acids to about 180 amino acids, about 10 amino acids to about 175 amino acids, about 10 amino acids to about 170 amino acids, about 10 amino acids to about 165 amino acids, about 10 amino acids to about 160 amino acids, about 10 amino acids to about 155 amino acids, about 10 amino acids to about 150 amino acids, about 10 amino acids to about 145 amino acids, about 10 amino acids to about 140 amino acids, about 10 amino acids to about 135 amino acids, about 10 amino acids to about 130 amino acids, about 10 amino acids to about 125 amino acids, about 10 amino acids to about 120 amino acids, about 10 amino acids to about 115 amino acids, about 10 amino acids to about 110 amino acids, about 10 amino acids to about 105 amino acids, about 10 amino acids to about 100 amino acids, about 10 amino acids to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 1000 amino acids, about 15 amino acids to about 950 amino acids, about 15 amino acids to about 900 amino acids, about 15 amino acids to about 850 amino acids, about 15 amino acids to about 800 amino acids, about 15 amino acids to about 750 amino acids, about 15 amino acids to about 700 amino acids, about 15 amino acids to about 650 amino acids, about 15 amino acids to about 600 amino acids, about 15 amino acids to about 550 amino acids, about 15 amino acids to about 500 amino acids, about 15 amino acids to about 450 amino acids, about 15 amino acids to about 400 amino acids, about 15 amino acids to about 350 amino acids, about 15 amino acids to about 300 amino acids, about 15 amino acids to about 280 amino acids, about 15 amino acids to about 260 amino acids, about 15 amino acids to about 240 amino acids, about 15 amino acids to about 220 amino acids, about 15 amino acids to about 200 amino acids, about 15 amino acids to about 195 amino acids, about 15 amino acids to about 190 amino acids, about 15 amino acids to about 185 amino acids, about 15 amino acids to about 180 amino acids, about 15 amino acids to about 175 amino acids, about 15 amino acids to about 170 amino acids, about 15 amino acids to about 165 amino acids, about 15 amino acids to about 160 amino acids, about 15 amino acids to about 155 amino acids, about 15 amino acids to about 150 amino acids, about 15 amino acids to about 145 amino acids, about 15 amino acids to about 140 amino acids, about 15 amino acids to about 135 amino acids, about 15 amino acids to about 130 amino acids, about 15 amino acids to about 125 amino acids, about 15 amino acids to about 120 amino acids, about 15 amino acids to about 115 amino acids, about 15 amino acids to about 110 amino acids, about 15 amino acids to about 105 amino acids, about 15 amino acids to about 100 amino acids, about 15 amino acids to about 95 amino acids, about 15 amino acids to about 90 amino acids, about 15 amino acids to about 85 amino acids, about 15 amino acids to about 80 amino acids, about 15 amino acids to about 75 amino acids, about 15 amino acids to about 70 amino acids, about 15 amino acids to about 65 amino acids, about 15 amino acids to about 60 amino acids, about 15 amino acids to about 55 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 1000 amino acids, about 20 amino acids to about 950 amino acids, about 20 amino acids to about 900 amino acids, about 20 amino acids to about 850 amino acids, about 20 amino acids to about 800 amino acids, about 20 amino acids to about 750 amino acids, about 20 amino acids to about 700 amino acids, about 20 amino acids to about 650 amino acids, about 20 amino acids to about 600 amino acids, about 20 amino acids to about 550 amino acids, about 20 amino acids to about 500 amino acids, about 20 amino acids to about 450 amino acids, about 20 amino acids to about 400 amino acids, about 20 amino acids to about 350 amino acids, about 20 amino acids to about 300 amino acids, about 20 amino acids to about 280 amino acids, about 20 amino acids to about 260 amino acids, about 20 amino acids to about 240 amino acids, about 20 amino acids to about 220 amino acids, about 20 amino acids to about 200 amino acids, about 20 amino acids to about 195 amino acids, about 20 amino acids to about 190 amino acids, about 20 amino acids to about 185 amino acids, about 20 amino acids to about 180 amino acids, about 20 amino acids to about 175 amino acids, about 20 amino acids to about 170 amino acids, about 20 amino acids to about 165 amino acids, about 20 amino acids to about 160 amino acids, about 20 amino acids to about 155 amino acids, about 20 amino acids to about 150 amino acids, about 20 amino acids to about 145 amino acids, about 20 amino acids to about 140 amino acids, about 20 amino acids to about 135 amino acids, about 20 amino acids to about 130 amino acids, about 20 amino acids to about 125 amino acids, about 20 amino acids to about 120 amino acids, about 20 amino acids to about 115 amino acids, about 20 amino acids to about 110 amino acids, about 20 amino acids to about 105 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 1000 amino acids, about 25 amino acids to about 950 amino acids, about 25 amino acids to about 900 amino acids, about 25 amino acids to about 850 amino acids, about 25 amino acids to about 800 amino acids, about 25 amino acids to about 750 amino acids, about 25 amino acids to about 700 amino acids, about 25 amino acids to about 650 amino acids, about 25 amino acids to about 600 amino acids, about 25 amino acids to about 550 amino acids, about 25 amino acids to about 500 amino acids, about 25 amino acids to about 450 amino acids, about 25 amino acids to about 400 amino acids, about 25 amino acids to about 350 amino acids, about 25 amino acids to about 300 amino acids, about 25 amino acids to about 280 amino acids, about 25 amino acids to about 260 amino acids, about 25 amino acids to about 240 amino acids, about 25 amino acids to about 220 amino acids, about 25 amino acids to about 200 amino acids, about 25 amino acids to about 195 amino acids, about 25 amino acids to about 190 amino acids, about 25 amino acids to about 185 amino acids, about 25 amino acids to about 180 amino acids, about 25 amino acids to about 175 amino acids, about 25 amino acids to about 170 amino acids, about 25 amino acids to about 165 amino acids, about 25 amino acids to about 160 amino acids, about 25 amino acids to about 155 amino acids, about 25 amino acids to about 150 amino acids, about 25 amino acids to about 145 amino acids, about 25 amino acids to about 140 amino acids, about 25 amino acids to about 135 amino acids, about 25 amino acids to about 130 amino acids, about 25 amino acids to about 125 amino acids, about 25 amino acids to about 120 amino acids, about 25 amino acids to about 115 amino acids, about 25 amino acids to about 110 amino acids, about 25 amino acids to about 105 amino acids, about 25 amino acids to about 100 amino acids, about 25 amino acids to about 95 amino acids, about 25 amino acids to about 90 amino acids, about 25 amino acids to about 85 amino acids, about 25 amino acids to about 80 amino acids, about 25 amino acids to about 75 amino acids, about 25 amino acids to about 70 amino acids, about 25 amino acids to about 65 amino acids, about 25 amino acids to about 60 amino acids, about 25 amino acids to about 55 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 1000 amino acids, about 30 amino acids to about 950 amino acids, about 30 amino acids to about 900 amino acids, about 30 amino acids to about 850 amino acids, about 30 amino acids to about 800 amino acids, about 30 amino acids to about 750 amino acids, about 30 amino acids to about 700 amino acids, about 30 amino acids to about 650 amino acids, about 30 amino acids to about 600 amino acids, about 30 amino acids to about 550 amino acids, about 30 amino acids to about 500 amino acids, about 30 amino acids to about 450 amino acids, about 30 amino acids to about 400 amino acids, about 30 amino acids to about 350 amino acids, about 30 amino acids to about 300 amino acids, about 30 amino acids to about 280 amino acids, about 30 amino acids to about 260 amino acids, about 30 amino acids to about 240 amino acids, about 30 amino acids to about 220 amino acids, about 30 amino acids to about 200 amino acids, about 30 amino acids to about 195 amino acids, about 30 amino acids to about 190 amino acids, about 30 amino acids to about 185 amino acids, about 30 amino acids to about 180 amino acids, about 30 amino acids to about 175 amino acids, about 30 amino acids to about 170 amino acids, about 30 amino acids to about 165 amino acids, about 30 amino acids to about 160 amino acids, about 30 amino acids to about 155 amino acids, about 30 amino acids to about 150 amino acids, about 30 amino acids to about 145 amino acids, about 30 amino acids to about 140 amino acids, about 30 amino acids to about 135 amino acids, about 30 amino acids to about 130 amino acids, about 30 amino acids to about 125 amino acids, about 30 amino acids to about 120 amino acids, about 30 amino acids to about 115 amino acids, about 30 amino acids to about 110 amino acids, about 30 amino acids to about 105 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 35 amino acids to about 1000 amino acids, about 35 amino acids to about 950 amino acids, about 35 amino acids to about 900 amino acids, about 35 amino acids to about 850 amino acids, about 35 amino acids to about 800 amino acids, about 35 amino acids to about 750 amino acids, about 35 amino acids to about 700 amino acids, about 35 amino acids to about 650 amino acids, about 35 amino acids to about 600 amino acids, about 35 amino acids to about 550 amino acids, about 35 amino acids to about 500 amino acids, about 35 amino acids to about 450 amino acids, about 35 amino acids to about 400 amino acids, about 35 amino acids to about 350 amino acids, about 35 amino acids to about 300 amino acids, about 35 amino acids to about 280 amino acids, about 35 amino acids to about 260 amino acids, about 35 amino acids to about 240 amino acids, about 35 amino acids to about 220 amino acids, about 35 amino acids to about 200 amino acids, about 35 amino acids to about 195 amino acids, about 35 amino acids to about 190 amino acids, about 35 amino acids to about 185 amino acids, about 35 amino acids to about 180 amino acids, about 35 amino acids to about 175 amino acids, about 35 amino acids to about 170 amino acids, about 35 amino acids to about 165 amino acids, about 35 amino acids to about 160 amino acids, about 35 amino acids to about 155 amino acids, about 35 amino acids to about 150 amino acids, about 35 amino acids to about 145 amino acids, about 35 amino acids to about 140 amino acids, about 35 amino acids to about 135 amino acids, about 35 amino acids to about 130 amino acids, about 35 amino acids to about 125 amino acids, about 35 amino acids to about 120 amino acids, about 35 amino acids to about 115 amino acids, about 35 amino acids to about 110 amino acids, about 35 amino acids to about 105 amino acids, about 35 amino acids to about 100 amino acids, about 35 amino acids to about 95 amino acids, about 35 amino acids to about 90 amino acids, about 35 amino acids to about 85 amino acids, about 35 amino acids to about 80 amino acids, about 35 amino acids to about 75 amino acids, about 35 amino acids to about 70 amino acids, about 35 amino acids to about 65 amino acids, about 35 amino acids to about 60 amino acids, about 35 amino acids to about 55 amino acids, about 35 amino acids to about 50 amino acids, about 35 amino acids to about 45 amino acids, about 35 amino acids to about 40 amino acids, about 40 amino acids to about 1000 amino acids, about 40 amino acids to about 950 amino acids, about 40 amino acids to about 900 amino acids, about 40 amino acids to about 850 amino acids, about 40 amino acids to about 800 amino acids, about 40 amino acids to about 750 amino acids, about 40 amino acids to about 700 amino acids, about 40 amino acids to about 650 amino acids, about 40 amino acids to about 600 amino acids, about 40 amino acids to about 550 amino acids, about 40 amino acids to about 500 amino acids, about 40 amino acids to about 450 amino acids, about 40 amino acids to about 400 amino acids, about 40 amino acids to about 350 amino acids, about 40 amino acids to about 300 amino acids, about 40 amino acids to about 280 amino acids, about 40 amino acids to about 260 amino acids, about 40 amino acids to about 240 amino acids, about 40 amino acids to about 220 amino acids, about 40 amino acids to about 200 amino acids, about 40 amino acids to about 195 amino acids, about 40 amino acids to about 190 amino acids, about 40 amino acids to about 185 amino acids, about 40 amino acids to about 180 amino acids, about 40 amino acids to about 175 amino acids, about 40 amino acids to about 170 amino acids, about 40 amino acids to about 165 amino acids, about 40 amino acids to about 160 amino acids, about 40 amino acids to about 155 amino acids, about 40 amino acids to about 150 amino acids, about 40 amino acids to about 145 amino acids, about 40 amino acids to about 140 amino acids, about 40 amino acids to about 135 amino acids, about 40 amino acids to about 130 amino acids, about 40 amino acids to about 125 amino acids, about 40 amino acids to about 120 amino acids, about 40 amino acids to about 115 amino acids, about 40 amino acids to about 110 amino acids, about 40 amino acids to about 105 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 45 amino acids to about 1000 amino acids, about 45 amino acids to about 950 amino acids, about 45 amino acids to about 900 amino acids, about 45 amino acids to about 850 amino acids, about 45 amino acids to about 800 amino acids, about 45 amino acids to about 750 amino acids, about 45 amino acids to about 700 amino acids, about 45 amino acids to about 650 amino acids, about 45 amino acids to about 600 amino acids, about 45 amino acids to about 550 amino acids, about 45 amino acids to about 500 amino acids, about 45 amino acids to about 450 amino acids, about 45 amino acids to about 400 amino acids, about 45 amino acids to about 350 amino acids, about 45 amino acids to about 300 amino acids, about 45 amino acids to about 280 amino acids, about 45 amino acids to about 260 amino acids, about 45 amino acids to about 240 amino acids, about 45 amino acids to about 220 amino acids, about 45 amino acids to about 200 amino acids, about 45 amino acids to about 195 amino acids, about 45 amino acids to about 190 amino acids, about 45 amino acids to about 185 amino acids, about 45 amino acids to about 180 amino acids, about 45 amino acids to about 175 amino acids, about 45 amino acids to about 170 amino acids, about 45 amino acids to about 165 amino acids, about 45 amino acids to about 160 amino acids, about 45 amino acids to about 155 amino acids, about 45 amino acids to about 150 amino acids, about 45 amino acids to about 145 amino acids, about 45 amino acids to about 140 amino acids, about 45 amino acids to about 135 amino acids, about 45 amino acids to about 130 amino acids, about 45 amino acids to about 125 amino acids, about 45 amino acids to about 120 amino acids, about 45 amino acids to about 115 amino acids, about 45 amino acids to about 110 amino acids, about 45 amino acids to about 105 amino acids, about 45 amino acids to about 100 amino acids, about 45 amino acids to about 95 amino acids, about 45 amino acids to about 90 amino acids, about 45 amino acids to about 85 amino acids, about 45 amino acids to about 80 amino acids, about 45 amino acids to about 75 amino acids, about 45 amino acids to about 70 amino acids, about 45 amino acids to about 65 amino acids, about 45 amino acids to about 60 amino acids, about 45 amino acids to about 55 amino acids, about 45 amino acids to about 50 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 280 amino acids, about 50 amino acids to about 260 amino acids, about 50 amino acids to about 240 amino acids, about 50 amino acids to about 220 amino acids, about 50 amino acids to about 200 amino acids, about 50 amino acids to about 195 amino acids, about 50 amino acids to about 190 amino acids, about 50 amino acids to about 185 amino acids, about 50 amino acids to about 180 amino acids, about 50 amino acids to about 175 amino acids, about 50 amino acids to about 170 amino acids, about 50 amino acids to about 165 amino acids, about 50 amino acids to about 160 amino acids, about 50 amino acids to about 155 amino acids, about 50 amino acids to about 150 amino acids, about 50 amino acids to about 145 amino acids, about 50 amino acids to about 140 amino acids, about 50 amino acids to about 135 amino acids, about 50 amino acids to about 130 amino acids, about 50 amino acids to about 125 amino acids, about 50 amino acids to about 120 amino acids, about 50 amino acids to about 115 amino acids, about 50 amino acids to about 110 amino acids, about 50 amino acids to about 105 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 55 amino acids to about 1000 amino acids, about 55 amino acids to about 950 amino acids, about 55 amino acids to about 900 amino acids, about 55 amino acids to about 850 amino acids, about 55 amino acids to about 800 amino acids, about 55 amino acids to about 750 amino acids, about 55 amino acids to about 700 amino acids, about 55 amino acids to about 650 amino acids, about 55 amino acids to about 600 amino acids, about 55 amino acids to about 550 amino acids, about 55 amino acids to about 500 amino acids, about 55 amino acids to about 450 amino acids, about 55 amino acids to about 400 amino acids, about 55 amino acids to about 350 amino acids, about 55 amino acids to about 300 amino acids, about 55 amino acids to about 280 amino acids, about 55 amino acids to about 260 amino acids, about 55 amino acids to about 240 amino acids, about 55 amino acids to about 220 amino acids, about 55 amino acids to about 200 amino acids, about 55 amino acids to about 195 amino acids, about 55 amino acids to about 190 amino acids, about 55 amino acids to about 185 amino acids, about 55 amino acids to about 180 amino acids, about 55 amino acids to about 175 amino acids, about 55 amino acids to about 170 amino acids, about 55 amino acids to about 165 amino acids, about 55 amino acids to about 160 amino acids, about 55 amino acids to about 155 amino acids, about 55 amino acids to about 150 amino acids, about 55 amino acids to about 145 amino acids, about 55 amino acids to about 140 amino acids, about 55 amino acids to about 135 amino acids, about 55 amino acids to about 130 amino acids, about 55 amino acids to about 125 amino acids, about 55 amino acids to about 120 amino acids, about 55 amino acids to about 115 amino acids, about 55 amino acids to about 110 amino acids, about 55 amino acids to about 105 amino acids, about 55 amino acids to about 100 amino acids, about 55 amino acids to about 95 amino acids, about 55 amino acids to about 90 amino acids, about 55 amino acids to about 85 amino acids, about 55 amino acids to about 80 amino acids, about 55 amino acids to about 75 amino acids, about 55 amino acids to about 70 amino acids, about 55 amino acids to about 65 amino acids, about 55 amino acids to about 60 amino acids, about 60 amino acids to about 1000 amino acids, about 60 amino acids to about 950 amino acids, about 60 amino acids to about 900 amino acids, about 60 amino acids to about 850 amino acids, about 60 amino acids to about 800 amino acids, about 60 amino acids to about 750 amino acids, about 60 amino acids to about 700 amino acids, about 60 amino acids to about 650 amino acids, about 60 amino acids to about 600 amino acids, about 60 amino acids to about 550 amino acids, about 60 amino acids to about 500 amino acids, about 60 amino acids to about 450 amino acids, about 60 amino acids to about 400 amino acids, about 60 amino acids to about 350 amino acids, about 60 amino acids to about 300 amino acids, about 60 amino acids to about 280 amino acids, about 60 amino acids to about 260 amino acids, about 60 amino acids to about 240 amino acids, about 60 amino acids to about 220 amino acids, about 60 amino acids to about 200 amino acids, about 60 amino acids to about 195 amino acids, about 60 amino acids to about 190 amino acids, about 60 amino acids to about 185 amino acids, about 60 amino acids to about 180 amino acids, about 60 amino acids to about 175 amino acids, about 60 amino acids to about 170 amino acids, about 60 amino acids to about 165 amino acids, about 60 amino acids to about 160 amino acids, about 60 amino acids to about 155 amino acids, about 60 amino acids to about 150 amino acids, about 60 amino acids to about 145 amino acids, about 60 amino acids to about 140 amino acids, about 60 amino acids to about 135 amino acids, about 60 amino acids to about 130 amino acids, about 60 amino acids to about 125 amino acids, about 60 amino acids to about 120 amino acids, about 60 amino acids to about 115 amino acids, about 60 amino acids to about 110 amino acids, about 60 amino acids to about 105 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 65 amino acids to about 1000 amino acids, about 65 amino acids to about 950 amino acids, about 65 amino acids to about 900 amino acids, about 65 amino acids to about 850 amino acids, about 65 amino acids to about 800 amino acids, about 65 amino acids to about 750 amino acids, about 65 amino acids to about 700 amino acids, about 65 amino acids to about 650 amino acids, about 65 amino acids to about 600 amino acids, about 65 amino acids to about 550 amino acids, about 65 amino acids to about 500 amino acids, about 65 amino acids to about 450 amino acids, about 65 amino acids to about 400 amino acids, about 65 amino acids to about 350 amino acids, about 65 amino acids to about 300 amino acids, about 65 amino acids to about 280 amino acids, about 65 amino acids to about 260 amino acids, about 65 amino acids to about 240 amino acids, about 65 amino acids to about 220 amino acids, about 65 amino acids to about 200 amino acids, about 65 amino acids to about 195 amino acids, about 65 amino acids to about 190 amino acids, about 65 amino acids to about 185 amino acids, about 65 amino acids to about 180 amino acids, about 65 amino acids to about 175 amino acids, about 65 amino acids to about 170 amino acids, about 65 amino acids to about 165 amino acids, about 65 amino acids to about 160 amino acids, about 65 amino acids to about 155 amino acids, about 65 amino acids to about 150 amino acids, about 65 amino acids to about 145 amino acids, about 65 amino acids to about 140 amino acids, about 65 amino acids to about 135 amino acids, about 65 amino acids to about 130 amino acids, about 65 amino acids to about 125 amino acids, about 65 amino acids to about 120 amino acids, about 65 amino acids to about 115 amino acids, about 65 amino acids to about 110 amino acids, about 65 amino acids to about 105 amino acids, about 65 amino acids to about 100 amino acids, about 65 amino acids to about 95 amino acids, about 65 amino acids to about 90 amino acids, about 65 amino acids to about 85 amino acids, about 65 amino acids to about 80 amino acids, about 65 amino acids to about 75 amino acids, about 65 amino acids to about 70 amino acids, about 70 amino acids to about 1000 amino acids, about 70 amino acids to about 950 amino acids, about 70 amino acids to about 900 amino acids, about 70 amino acids to about 850 amino acids, about 70 amino acids to about 800 amino acids, about 70 amino acids to about 750 amino acids, about 70 amino acids to about 700 amino acids, about 70 amino acids to about 650 amino acids, about 70 amino acids to about 600 amino acids, about 70 amino acids to about 550 amino acids, about 70 amino acids to about 500 amino acids, about 70 amino acids to about 450 amino acids, about 70 amino acids to about 400 amino acids, about 70 amino acids to about 350 amino acids, about 70 amino acids to about 300 amino acids, about 70 amino acids to about 280 amino acids, about 70 amino acids to about 260 amino acids, about 70 amino acids to about 240 amino acids, about 70 amino acids to about 220 amino acids, about 70 amino acids to about 200 amino acids, about 70 amino acids to about 195 amino acids, about 70 amino acids to about 190 amino acids, about 70 amino acids to about 185 amino acids, about 70 amino acids to about 180 amino acids, about 70 amino acids to about 175 amino acids, about 70 amino acids to about 170 amino acids, about 70 amino acids to about 165 amino acids, about 70 amino acids to about 160 amino acids, about 70 amino acids to about 155 amino acids, about 70 amino acids to about 150 amino acids, about 70 amino acids to about 145 amino acids, about 70 amino acids to about 140 amino acids, about 70 amino acids to about 135 amino acids, about 70 amino acids to about 130 amino acids, about 70 amino acids to about 125 amino acids, about 70 amino acids to about 120 amino acids, about 70 amino acids to about 115 amino acids, about 70 amino acids to about 110 amino acids, about 70 amino acids to about 105 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 70 amino acids to about 75 amino acids, about 75 amino acids to about 1000 amino acids, about 75 amino acids to about 950 amino acids, about 75 amino acids to about 900 amino acids, about 75 amino acids to about 850 amino acids, about 75 amino acids to about 800 amino acids, about 75 amino acids to about 750 amino acids, about 75 amino acids to about 700 amino acids, about 75 amino acids to about 650 amino acids, about 75 amino acids to about 600 amino acids, about 75 amino acids to about 550 amino acids, about 75 amino acids to about 500 amino acids, about 75 amino acids to about 450 amino acids, about 75 amino acids to about 400 amino acids, about 75 amino acids to about 350 amino acids, about 75 amino acids to about 300 amino acids, about 75 amino acids to about 280 amino acids, about 75 amino acids to about 260 amino acids, about 75 amino acids to about 240 amino acids, about 75 amino acids to about 220 amino acids, about 75 amino acids to about 200 amino acids, about 75 amino acids to about 195 amino acids, about 75 amino acids to about 190 amino acids, about 75 amino acids to about 185 amino acids, about 75 amino acids to about 180 amino acids, about 75 amino acids to about 175 amino acids, about 75 amino acids to about 170 amino acids, about 75 amino acids to about 165 amino acids, about 75 amino acids to about 160 amino acids, about 75 amino acids to about 155 amino acids, about 75 amino acids to about 150 amino acids, about 75 amino acids to about 145 amino acids, about 75 amino acids to about 140 amino acids, about 75 amino acids to about 135 amino acids, about 75 amino acids to about 130 amino acids, about 75 amino acids to about 125 amino acids, about 75 amino acids to about 120 amino acids, about 75 amino acids to about 115 amino acids, about 75 amino acids to about 110 amino acids, about 75 amino acids to about 105 amino acids, about 75 amino acids to about 100 amino acids, about 75 amino acids to about 95 amino acids, about 75 amino acids to about 90 amino acids, about 75 amino acids to about 85 amino acids, about 75 amino acids to about 80 amino acids, about 80 amino acids to about 1000 amino acids, about 80 amino acids to about 950 amino acids, about 80 amino acids to about 900 amino acids, about 80 amino acids to about 850 amino acids, about 80 amino acids to about 800 amino acids, about 80 amino acids to about 750 amino acids, about 80 amino acids to about 700 amino acids, about 80 amino acids to about 650 amino acids, about 80 amino acids to about 600 amino acids, about 80 amino acids to about 550 amino acids, about 80 amino acids to about 500 amino acids, about 80 amino acids to about 450 amino acids, about 80 amino acids to about 400 amino acids, about 80 amino acids to about 350 amino acids, about 80 amino acids to about 300 amino acids, about 80 amino acids to about 280 amino acids, about 80 amino acids to about 260 amino acids, about 80 amino acids to about 240 amino acids, about 80 amino acids to about 220 amino acids, about 80 amino acids to about 200 amino acids, about 80 amino acids to about 195 amino acids, about 80 amino acids to about 190 amino acids, about 80 amino acids to about 185 amino acids, about 80 amino acids to about 180 amino acids, about 80 amino acids to about 175 amino acids, about 80 amino acids to about 170 amino acids, about 80 amino acids to about 165 amino acids, about 80 amino acids to about 160 amino acids, about 80 amino acids to about 155 amino acids, about 80 amino acids to about 150 amino acids, about 80 amino acids to about 145 amino acids, about 80 amino acids to about 140 amino acids, about 80 amino acids to about 135 amino acids, about 80 amino acids to about 130 amino acids, about 80 amino acids to about 125 amino acids, about 80 amino acids to about 120 amino acids, about 80 amino acids to about 115 amino acids, about 80 amino acids to about 110 amino acids, about 80 amino acids to about 105 amino acids, about 80 amino acids to about 100 amino acids, about 80 amino acids to about 95 amino acids, about 80 amino acids to about 90 amino acids, about 80 amino acids to about 85 amino acids, about 85 amino acids to about 1000 amino acids, about 85 amino acids to about 950 amino acids, about 85 amino acids to about 900 amino acids, about 85 amino acids to about 850 amino acids, about 85 amino acids to about 800 amino acids, about 85 amino acids to about 750 amino acids, about 85 amino acids to about 700 amino acids, about 85 amino acids to about 650 amino acids, about 85 amino acids to about 600 amino acids, about 85 amino acids to about 550 amino acids, about 85 amino acids to about 500 amino acids, about 85 amino acids to about 450 amino acids, about 85 amino acids to about 400 amino acids, about 85 amino acids to about 350 amino acids, about 85 amino acids to about 300 amino acids, about 85 amino acids to about 280 amino acids, about 85 amino acids to about 260 amino acids, about 85 amino acids to about 240 amino acids, about 85 amino acids to about 220 amino acids, about 85 amino acids to about 200 amino acids, about 85 amino acids to about 195 amino acids, about 85 amino acids to about 190 amino acids, about 85 amino acids to about 185 amino acids, about 85 amino acids to about 180 amino acids, about 85 amino acids to about 175 amino acids, about 85 amino acids to about 170 amino acids, about 85 amino acids to about 165 amino acids, about 85 amino acids to about 160 amino acids, about 85 amino acids to about 155 amino acids, about 85 amino acids to about 150 amino acids, about 85 amino acids to about 145 amino acids, about 85 amino acids to about 140 amino acids, about 85 amino acids to about 135 amino acids, about 85 amino acids to about 130 amino acids, about 85 amino acids to about 125 amino acids, about 85 amino acids to about 120 amino acids, about 85 amino acids to about 115 amino acids, about 85 amino acids to about 110 amino acids, about 85 amino acids to about 105 amino acids, about 85 amino acids to about 100 amino acids, about 85 amino acids to about 95 amino acids, about 85 amino acids to about 90 amino acids, about 90 amino acids to about 1000 amino acids, about 90 amino acids to about 950 amino acids, about 90 amino acids to about 900 amino acids, about 90 amino acids to about 850 amino acids, about 90 amino acids to about 800 amino acids, about 90 amino acids to about 750 amino acids, about 90 amino acids to about 700 amino acids, about 90 amino acids to about 650 amino acids, about 90 amino acids to about 600 amino acids, about 90 amino acids to about 550 amino acids, about 90 amino acids to about 500 amino acids, about 90 amino acids to about 450 amino acids, about 90 amino acids to about 400 amino acids, about 90 amino acids to about 350 amino acids, about 90 amino acids to about 300 amino acids, about 90 amino acids to about 280 amino acids, about 90 amino acids to about 260 amino acids, about 90 amino acids to about 240 amino acids, about 90 amino acids to about 220 amino acids, about 90 amino acids to about 200 amino acids, about 90 amino acids to about 195 amino acids, about 90 amino acids to about 190 amino acids, about 90 amino acids to about 185 amino acids, about 90 amino acids to about 180 amino acids, about 90 amino acids to about 175 amino acids, about 90 amino acids to about 170 amino acids, about 90 amino acids to about 165 amino acids, about 90 amino acids to about 160 amino acids, about 90 amino acids to about 155 amino acids, about 90 amino acids to about 150 amino acids, about 90 amino acids to about 145 amino acids, about 90 amino acids to about 140 amino acids, about 90 amino acids to about 135 amino acids, about 90 amino acids to about 130 amino acids, about 90 amino acids to about 125 amino acids, about 90 amino acids to about 120 amino acids, about 90 amino acids to about 115 amino acids, about 90 amino acids to about 110 amino acids, about 90 amino acids to about 105 amino acids, about 90 amino acids to about 100 amino acids, about 90 amino acids to about 95 amino acids, about 95 amino acids to about 1000 amino acids, about 95 amino acids to about 950 amino acids, about 95 amino acids to about 900 amino acids, about 95 amino acids to about 850 amino acids, about 95 amino acids to about 800 amino acids, about 95 amino acids to about 750 amino acids, about 95 amino acids to about 700 amino acids, about 95 amino acids to about 650 amino acids, about 95 amino acids to about 600 amino acids, about 95 amino acids to about 550 amino acids, about 95 amino acids to about 500 amino acids, about 95 amino acids to about 450 amino acids, about 95 amino acids to about 400 amino acids, about 95 amino acids to about 350 amino acids, about 95 amino acids to about 300 amino acids, about 95 amino acids to about 280 amino acids, about 95 amino acids to about 260 amino acids, about 95 amino acids to about 240 amino acids, about 95 amino acids to about 220 amino acids, about 95 amino acids to about 200 amino acids, about 95 amino acids to about 195 amino acids, about 95 amino acids to about 190 amino acids, about 95 amino acids to about 185 amino acids, about 95 amino acids to about 180 amino acids, about 95 amino acids to about 175 amino acids, about 95 amino acids to about 170 amino acids, about 95 amino acids to about 165 amino acids, about 95 amino acids to about 160 amino acids, about 95 amino acids to about 155 amino acids, about 95 amino acids to about 150 amino acids, about 95 amino acids to about 145 amino acids, about 95 amino acids to about 140 amino acids, about 95 amino acids to about 135 amino acids, about 95 amino acids to about 130 amino acids, about 95 amino acids to about 125 amino acids, about 95 amino acids to about 120 amino acids, about 95 amino acids to about 115 amino acids, about 95 amino acids to about 110 amino acids, about 95 amino acids to about 105 amino acids, about 95 amino acids to about 100 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 280 amino acids, about 100 amino acids to about 260 amino acids, about 100 amino acids to about 240 amino acids, about 100 amino acids to about 220 amino acids, about 100 amino acids to about 200 amino acids, about 100 amino acids to about 195 amino acids, about 100 amino acids to about 190 amino acids, about 100 amino acids to about 185 amino acids, about 100 amino acids to about 180 amino acids, about 100 amino acids to about 175 amino acids, about 100 amino acids to about 170 amino acids, about 100 amino acids to about 165 amino acids, about 100 amino acids to about 160 amino acids, about 100 amino acids to about 155 amino acids, about 100 amino acids to about 150 amino acids, about 100 amino acids to about 145 amino acids, about 100 amino acids to about 140 amino acids, about 100 amino acids to about 135 amino acids, about 100 amino acids to about 130 amino acids, about 100 amino acids to about 125 amino acids, about 100 amino acids to about 120 amino acids, about 100 amino acids to about 115 amino acids, about 100 amino acids to about 110 amino acids, about 100 amino acids to about 105 amino acids, about 105 amino acids to about 1000 amino acids, about 105 amino acids to about 950 amino acids, about 105 amino acids to about 900 amino acids, about 105 amino acids to about 850 amino acids, about 105 amino acids to about 800 amino acids, about 105 amino acids to about 750 amino acids, about 105 amino acids to about 700 amino acids, about 105 amino acids to about 650 amino acids, about 105 amino acids to about 600 amino acids, about 105 amino acids to about 550 amino acids, about 105 amino acids to about 500 amino acids, about 105 amino acids to about 450 amino acids, about 105 amino acids to about 400 amino acids, about 105 amino acids to about 350 amino acids, about 105 amino acids to about 300 amino acids, about 105 amino acids to about 280 amino acids, about 105 amino acids to about 260 amino acids, about 105 amino acids to about 240 amino acids, about 105 amino acids to about 220 amino acids, about 105 amino acids to about 200 amino acids, about 105 amino acids to about 195 amino acids, about 105 amino acids to about 190 amino acids, about 105 amino acids to about 185 amino acids, about 105 amino acids to about 180 amino acids, about 105 amino acids to about 175 amino acids, about 105 amino acids to about 170 amino acids, about 105 amino acids to about 165 amino acids, about 105 amino acids to about 160 amino acids, about 105 amino acids to about 155 amino acids, about 105 amino acids to about 150 amino acids, about 105 amino acids to about 145 amino acids, about 105 amino acids to about 140 amino acids, about 105 amino acids to about 135 amino acids, about 105 amino acids to about 130 amino acids, about 105 amino acids to about 125 amino acids, about 105 amino acids to about 120 amino acids, about 105 amino acids to about 115 amino acids, about 105 amino acids to about 110 amino acids, about 110 amino acids to about 1000 amino acids, about 110 amino acids to about 950 amino acids, about 110 amino acids to about 900 amino acids, about 110 amino acids to about 850 amino acids, about 110 amino acids to about 800 amino acids, about 110 amino acids to about 750 amino acids, about 110 amino acids to about 700 amino acids, about 110 amino acids to about 650 amino acids, about 110 amino acids to about 600 amino acids, about 110 amino acids to about 550 amino acids, about 110 amino acids to about 500 amino acids, about 110 amino acids to about 450 amino acids, about 110 amino acids to about 400 amino acids, about 110 amino acids to about 350 amino acids, about 110 amino acids to about 300 amino acids, about 110 amino acids to about 280 amino acids, about 110 amino acids to about 260 amino acids, about 110 amino acids to about 240 amino acids, about 110 amino acids to about 220 amino acids, about 110 amino acids to about 200 amino acids, about 110 amino acids to about 195 amino acids, about 110 amino acids to about 190 amino acids, about 110 amino acids to about 185 amino acids, about 110 amino acids to about 180 amino acids, about 110 amino acids to about 175 amino acids, about 110 amino acids to about 170 amino acids, about 110 amino acids to about 165 amino acids, about 110 amino acids to about 160 amino acids, about 110 amino acids to about 155 amino acids, about 110 amino acids to about 150 amino acids, about 110 amino acids to about 145 amino acids, about 110 amino acids to about 140 amino acids, about 110 amino acids to about 135 amino acids, about 110 amino acids to about 130 amino acids, about 110 amino acids to about 125 amino acids, about 110 amino acids to about 120 amino acids, about 110 amino acids to about 115 amino acids, about 115 amino acids to about 1000 amino acids, about 115 amino acids to about 950 amino acids, about 115 amino acids to about 900 amino acids, about 115 amino acids to about 850 amino acids, about 115 amino acids to about 800 amino acids, about 115 amino acids to about 750 amino acids, about 115 amino acids to about 700 amino acids, about 115 amino acids to about 650 amino acids, about 115 amino acids to about 600 amino acids, about 115 amino acids to about 550 amino acids, about 115 amino acids to about 500 amino acids, about 115 amino acids to about 450 amino acids, about 115 amino acids to about 400 amino acids, about 115 amino acids to about 350 amino acids, about 115 amino acids to about 300 amino acids, about 115 amino acids to about 280 amino acids, about 115 amino acids to about 260 amino acids, about 115 amino acids to about 240 amino acids, about 115 amino acids to about 220 amino acids, about 115 amino acids to about 200 amino acids, about 115 amino acids to about 195 amino acids, about 115 amino acids to about 190 amino acids, about 115 amino acids to about 185 amino acids, about 115 amino acids to about 180 amino acids, about 115 amino acids to about 175 amino acids, about 115 amino acids to about 170 amino acids, about 115 amino acids to about 165 amino acids, about 115 amino acids to about 160 amino acids, about 115 amino acids to about 155 amino acids, about 115 amino acids to about 150 amino acids, about 115 amino acids to about 145 amino acids, about 115 amino acids to about 140 amino acids, about 115 amino acids to about 135 amino acids, about 115 amino acids to about 130 amino acids, about 115 amino acids to about 125 amino acids, about 115 amino acids to about 120 amino acids, about 120 amino acids to about 1000 amino acids, about 120 amino acids to about 950 amino acids, about 120 amino acids to about 900 amino acids, about 120 amino acids to about 850 amino acids, about 120 amino acids to about 800 amino acids, about 120 amino acids to about 750 amino acids, about 120 amino acids to about 700 amino acids, about 120 amino acids to about 650 amino acids, about 120 amino acids to about 600 amino acids, about 120 amino acids to about 550 amino acids, about 120 amino acids to about 500 amino acids, about 120 amino acids to about 450 amino acids, about 120 amino acids to about 400 amino acids, about 120 amino acids to about 350 amino acids, about 120 amino acids to about 300 amino acids, about 120 amino acids to about 280 amino acids, about 120 amino acids to about 260 amino acids, about 120 amino acids to about 240 amino acids, about 120 amino acids to about 220 amino acids, about 120 amino acids to about 200 amino acids, about 120 amino acids to about 195 amino acids, about 120 amino acids to about 190 amino acids, about 120 amino acids to about 185 amino acids, about 120 amino acids to about 180 amino acids, about 120 amino acids to about 175 amino acids, about 120 amino acids to about 170 amino acids, about 120 amino acids to about 165 amino acids, about 120 amino acids to about 160 amino acids, about 120 amino acids to about 155 amino acids, about 120 amino acids to about 150 amino acids, about 120 amino acids to about 145 amino acids, about 120 amino acids to about 140 amino acids, about 120 amino acids to about 135 amino acids, about 120 amino acids to about 130 amino acids, about 120 amino acids to about 125 amino acids, about 125 amino acids to about 1000 amino acids, about 125 amino acids to about 950 amino acids, about 125 amino acids to about 900 amino acids, about 125 amino acids to about 850 amino acids, about 125 amino acids to about 800 amino acids, about 125 amino acids to about 750 amino acids, about 125 amino acids to about 700 amino acids, about 125 amino acids to about 650 amino acids, about 125 amino acids to about 600 amino acids, about 125 amino acids to about 550 amino acids, about 125 amino acids to about 500 amino acids, about 125 amino acids to about 450 amino acids, about 125 amino acids to about 400 amino acids, about 125 amino acids to about 350 amino acids, about 125 amino acids to about 300 amino acids, about 125 amino acids to about 280 amino acids, about 125 amino acids to about 260 amino acids, about 125 amino acids to about 240 amino acids, about 125 amino acids to about 220 amino acids, about 125 amino acids to about 200 amino acids, about 125 amino acids to about 195 amino acids, about 125 amino acids to about 190 amino acids, about 125 amino acids to about 185 amino acids, about 125 amino acids to about 180 amino acids, about 125 amino acids to about 175 amino acids, about 125 amino acids to about 170 amino acids, about 125 amino acids to about 165 amino acids, about 125 amino acids to about 160 amino acids, about 125 amino acids to about 155 amino acids, about 125 amino acids to about 150 amino acids, about 125 amino acids to about 145 amino acids, about 125 amino acids to about 140 amino acids, about 125 amino acids to about 135 amino acids, about 125 amino acids to about 130 amino acids, about 130 amino acids to about 1000 amino acids, about 130 amino acids to about 950 amino acids, about 130 amino acids to about 900 amino acids, about 130 amino acids to about 850 amino acids, about 130 amino acids to about 800 amino acids, about 130 amino acids to about 750 amino acids, about 130 amino acids to about 700 amino acids, about 130 amino acids to about 650 amino acids, about 130 amino acids to about 600 amino acids, about 130 amino acids to about 550 amino acids, about 130 amino acids to about 500 amino acids, about 130 amino acids to about 450 amino acids, about 130 amino acids to about 400 amino acids, about 130 amino acids to about 350 amino acids, about 130 amino acids to about 300 amino acids, about 130 amino acids to about 280 amino acids, about 130 amino acids to about 260 amino acids, about 130 amino acids to about 240 amino acids, about 130 amino acids to about 220 amino acids, about 130 amino acids to about 200 amino acids, about 130 amino acids to about 195 amino acids, about 130 amino acids to about 190 amino acids, about 130 amino acids to about 185 amino acids, about 130 amino acids to about 180 amino acids, about 130 amino acids to about 175 amino acids, about 130 amino acids to about 170 amino acids, about 130 amino acids to about 165 amino acids, about 130 amino acids to about 160 amino acids, about 130 amino acids to about 155 amino acids, about 130 amino acids to about 150 amino acids, about 130 amino acids to about 145 amino acids, about 130 amino acids to about 140 amino acids, about 130 amino acids to about 135 amino acids, about 135 amino acids to about 1000 amino acids, about 135 amino acids to about 950 amino acids, about 135 amino acids to about 900 amino acids, about 135 amino acids to about 850 amino acids, about 135 amino acids to about 800 amino acids, about 135 amino acids to about 750 amino acids, about 135 amino acids to about 700 amino acids, about 135 amino acids to about 650 amino acids, about 135 amino acids to about 600 amino acids, about 135 amino acids to about 550 amino acids, about 135 amino acids to about 500 amino acids, about 135 amino acids to about 450 amino acids, about 135 amino acids to about 400 amino acids, about 135 amino acids to about 350 amino acids, about 135 amino acids to about 300 amino acids, about 135 amino acids to about 280 amino acids, about 135 amino acids to about 260 amino acids, about 135 amino acids to about 240 amino acids, about 135 amino acids to about 220 amino acids, about 135 amino acids to about 200 amino acids, about 135 amino acids to about 195 amino acids, about 135 amino acids to about 190 amino acids, about 135 amino acids to about 185 amino acids, about 135 amino acids to about 180 amino acids, about 135 amino acids to about 175 amino acids, about 135 amino acids to about 170 amino acids, about 135 amino acids to about 165 amino acids, about 135 amino acids to about 160 amino acids, about 135 amino acids to about 155 amino acids, about 135 amino acids to about 150 amino acids, about 135 amino acids to about 145 amino acids, about 135 amino acids to about 140 amino acids, about 140 amino acids to about 1000 amino acids, about 140 amino acids to about 950 amino acids, about 140 amino acids to about 900 amino acids, about 140 amino acids to about 850 amino acids, about 140 amino acids to about 800 amino acids, about 140 amino acids to about 750 amino acids, about 140 amino acids to about 700 amino acids, about 140 amino acids to about 650 amino acids, about 140 amino acids to about 600 amino acids, about 140 amino acids to about 550 amino acids, about 140 amino acids to about 500 amino acids, about 140 amino acids to about 450 amino acids, about 140 amino acids to about 400 amino acids, about 140 amino acids to about 350 amino acids, about 140 amino acids to about 300 amino acids, about 140 amino acids to about 280 amino acids, about 140 amino acids to about 260 amino acids, about 140 amino acids to about 240 amino acids, about 140 amino acids to about 220 amino acids, about 140 amino acids to about 200 amino acids, about 140 amino acids to about 195 amino acids, about 140 amino acids to about 190 amino acids, about 140 amino acids to about 185 amino acids, about 140 amino acids to about 180 amino acids, about 140 amino acids to about 175 amino acids, about 140 amino acids to about 170 amino acids, about 140 amino acids to about 165 amino acids, about 140 amino acids to about 160 amino acids, about 140 amino acids to about 155 amino acids, about 140 amino acids to about 150 amino acids, about 140 amino acids to about 145 amino acids, about 145 amino acids to about 1000 amino acids, about 145 amino acids to about 950 amino acids, about 145 amino acids to about 900 amino acids, about 145 amino acids to about 850 amino acids, about 145 amino acids to about 800 amino acids, about 145 amino acids to about 750 amino acids, about 145 amino acids to about 700 amino acids, about 145 amino acids to about 650 amino acids, about 145 amino acids to about 600 amino acids, about 145 amino acids to about 550 amino acids, about 145 amino acids to about 500 amino acids, about 145 amino acids to about 450 amino acids, about 145 amino acids to about 400 amino acids, about 145 amino acids to about 350 amino acids, about 145 amino acids to about 300 amino acids, about 145 amino acids to about 280 amino acids, about 145 amino acids to about 260 amino acids, about 145 amino acids to about 240 amino acids, about 145 amino acids to about 220 amino acids, about 145 amino acids to about 200 amino acids, about 145 amino acids to about 195 amino acids, about 145 amino acids to about 190 amino acids, about 145 amino acids to about 185 amino acids, about 145 amino acids to about 180 amino acids, about 145 amino acids to about 175 amino acids, about 145 amino acids to about 170 amino acids, about 145 amino acids to about 165 amino acids, about 145 amino acids to about 160 amino acids, about 145 amino acids to about 155 amino acids, about 145 amino acids to about 150 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 280 amino acids, about 150 amino acids to about 260 amino acids, about 150 amino acids to about 240 amino acids, about 150 amino acids to about 220 amino acids, about 150 amino acids to about 200 amino acids, about 150 amino acids to about 195 amino acids, about 150 amino acids to about 190 amino acids, about 150 amino acids to about 185 amino acids, about 150 amino acids to about 180 amino acids, about 150 amino acids to about 175 amino acids, about 150 amino acids to about 170 amino acids, about 150 amino acids to about 165 amino acids, about 150 amino acids to about 160 amino acids, about 150 amino acids to about 155 amino acids, about 155 amino acids to about 1000 amino acids, about 155 amino acids to about 950 amino acids, about 155 amino acids to about 900 amino acids, about 155 amino acids to about 850 amino acids, about 155 amino acids to about 800 amino acids, about 155 amino acids to about 750 amino acids, about 155 amino acids to about 700 amino acids, about 155 amino acids to about 650 amino acids, about 155 amino acids to about 600 amino acids, about 155 amino acids to about 550 amino acids, about 155 amino acids to about 500 amino acids, about 155 amino acids to about 450 amino acids, about 155 amino acids to about 400 amino acids, about 155 amino acids to about 350 amino acids, about 155 amino acids to about 300 amino acids, about 155 amino acids to about 280 amino acids, about 155 amino acids to about 260 amino acids, about 155 amino acids to about 240 amino acids, about 155 amino acids to about 220 amino acids, about 155 amino acids to about 200 amino acids, about 155 amino acids to about 195 amino acids, about 155 amino acids to about 190 amino acids, about 155 amino acids to about 185 amino acids, about 155 amino acids to about 180 amino acids, about 155 amino acids to about 175 amino acids, about 155 amino acids to about 170 amino acids, about 155 amino acids to about 165 amino acids, about 155 amino acids to about 160 amino acids, about 160 amino acids to about 1000 amino acids, about 160 amino acids to about 950 amino acids, about 160 amino acids to about 900 amino acids, about 160 amino acids to about 850 amino acids, about 160 amino acids to about 800 amino acids, about 160 amino acids to about 750 amino acids, about 160 amino acids to about 700 amino acids, about 160 amino acids to about 650 amino acids, about 160 amino acids to about 600 amino acids, about 160 amino acids to about 550 amino acids, about 160 amino acids to about 500 amino acids, about 160 amino acids to about 450 amino acids, about 160 amino acids to about 400 amino acids, about 160 amino acids to about 350 amino acids, about 160 amino acids to about 300 amino acids, about 160 amino acids to about 280 amino acids, about 160 amino acids to about 260 amino acids, about 160 amino acids to about 240 amino acids, about 160 amino acids to about 220 amino acids, about 160 amino acids to about 200 amino acids, about 160 amino acids to about 195 amino acids, about 160 amino acids to about 190 amino acids, about 160 amino acids to about 185 amino acids, about 160 amino acids to about 180 amino acids, about 160 amino acids to about 175 amino acids, about 160 amino acids to about 170 amino acids, about 160 amino acids to about 165 amino acids, about 165 amino acids to about 1000 amino acids, about 165 amino acids to about 950 amino acids, about 165 amino acids to about 900 amino acids, about 165 amino acids to about 850 amino acids, about 165 amino acids to about 800 amino acids, about 165 amino acids to about 750 amino acids, about 165 amino acids to about 700 amino acids, about 165 amino acids to about 650 amino acids, about 165 amino acids to about 600 amino acids, about 165 amino acids to about 550 amino acids, about 165 amino acids to about 500 amino acids, about 165 amino acids to about 450 amino acids, about 165 amino acids to about 400 amino acids, about 165 amino acids to about 350 amino acids, about 165 amino acids to about 300 amino acids, about 165 amino acids to about 280 amino acids, about 165 amino acids to about 260 amino acids, about 165 amino acids to about 240 amino acids, about 165 amino acids to about 220 amino acids, about 165 amino acids to about 200 amino acids, about 165 amino acids to about 195 amino acids, about 165 amino acids to about 190 amino acids, about 165 amino acids to about 185 amino acids, about 165 amino acids to about 180 amino acids, about 165 amino acids to about 175 amino acids, about 165 amino acids to about 170 amino acids, about 170 amino acids to about 1000 amino acids, about 170 amino acids to about 950 amino acids, about 170 amino acids to about 900 amino acids, about 170 amino acids to about 850 amino acids, about 170 amino acids to about 800 amino acids, about 170 amino acids to about 750 amino acids, about 170 amino acids to about 700 amino acids, about 170 amino acids to about 650 amino acids, about 170 amino acids to about 600 amino acids, about 170 amino acids to about 550 amino acids, about 170 amino acids to about 500 amino acids, about 170 amino acids to about 450 amino acids, about 170 amino acids to about 400 amino acids, about 170 amino acids to about 350 amino acids, about 170 amino acids to about 300 amino acids, about 170 amino acids to about 280 amino acids, about 170 amino acids to about 260 amino acids, about 170 amino acids to about 240 amino acids, about 170 amino acids to about 220 amino acids, about 170 amino acids to about 200 amino acids, about 170 amino acids to about 195 amino acids, about 170 amino acids to about 190 amino acids, about 170 amino acids to about 185 amino acids, about 170 amino acids to about 180 amino acids, about 170 amino acids to about 175 amino acids, about 175 amino acids to about 1000 amino acids, about 175 amino acids to about 950 amino acids, about 175 amino acids to about 900 amino acids, about 175 amino acids to about 850 amino acids, about 175 amino acids to about 800 amino acids, about 175 amino acids to about 750 amino acids, about 175 amino acids to about 700 amino acids, about 175 amino acids to about 650 amino acids, about 175 amino acids to about 600 amino acids, about 175 amino acids to about 550 amino acids, about 175 amino acids to about 500 amino acids, about 175 amino acids to about 450 amino acids, about 175 amino acids to about 400 amino acids, about 175 amino acids to about 350 amino acids, about 175 amino acids to about 300 amino acids, about 175 amino acids to about 280 amino acids, about 175 amino acids to about 260 amino acids, about 175 amino acids to about 240 amino acids, about 175 amino acids to about 220 amino acids, about 175 amino acids to about 200 amino acids, about 175 amino acids to about 195 amino acids, about 175 amino acids to about 190 amino acids, about 175 amino acids to about 185 amino acids, about 175 amino acids to about 180 amino acids, about 180 amino acids to about 1000 amino acids, about 180 amino acids to about 950 amino acids, about 180 amino acids to about 900 amino acids, about 180 amino acids to about 850 amino acids, about 180 amino acids to about 800 amino acids, about 180 amino acids to about 750 amino acids, about 180 amino acids to about 700 amino acids, about 180 amino acids to about 650 amino acids, about 180 amino acids to about 600 amino acids, about 180 amino acids to about 550 amino acids, about 180 amino acids to about 500 amino acids, about 180 amino acids to about 450 amino acids, about 180 amino acids to about 400 amino acids, about 180 amino acids to about 350 amino acids, about 180 amino acids to about 300 amino acids, about 180 amino acids to about 280 amino acids, about 180 amino acids to about 260 amino acids, about 180 amino acids to about 240 amino acids, about 180 amino acids to about 220 amino acids, about 180 amino acids to about 200 amino acids, about 180 amino acids to about 195 amino acids, about 180 amino acids to about 190 amino acids, about 180 amino acids to about 185 amino acids, about 185 amino acids to about 1000 amino acids, about 185 amino acids to about 950 amino acids, about 185 amino acids to about 900 amino acids, about 185 amino acids to about 850 amino acids, about 185 amino acids to about 800 amino acids, about 185 amino acids to about 750 amino acids, about 185 amino acids to about 700 amino acids, about 185 amino acids to about 650 amino acids, about 185 amino acids to about 600 amino acids, about 185 amino acids to about 550 amino acids, about 185 amino acids to about 500 amino acids, about 185 amino acids to about 450 amino acids, about 185 amino acids to about 400 amino acids, about 185 amino acids to about 350 amino acids, about 185 amino acids to about 300 amino acids, about 185 amino acids to about 280 amino acids, about 185 amino acids to about 260 amino acids, about 185 amino acids to about 240 amino acids, about 185 amino acids to about 220 amino acids, about 185 amino acids to about 200 amino acids, about 185 amino acids to about 195 amino acids, about 185 amino acids to about 190 amino acids, about 190 amino acids to about 1000 amino acids, about 190 amino acids to about 950 amino acids, about 190 amino acids to about 900 amino acids, about 190 amino acids to about 850 amino acids, about 190 amino acids to about 800 amino acids, about 190 amino acids to about 750 amino acids, about 190 amino acids to about 700 amino acids, about 190 amino acids to about 650 amino acids, about 190 amino acids to about 600 amino acids, about 190 amino acids to about 550 amino acids, about 190 amino acids to about 500 amino acids, about 190 amino acids to about 450 amino acids, about 190 amino acids to about 400 amino acids, about 190 amino acids to about 350 amino acids, about 190 amino acids to about 300 amino acids, about 190 amino acids to about 280 amino acids, about 190 amino acids to about 260 amino acids, about 190 amino acids to about 240 amino acids, about 190 amino acids to about 220 amino acids, about 190 amino acids to about 200 amino acids, about 190 amino acids to about 195 amino acids, about 195 amino acids to about 1000 amino acids, about 195 amino acids to about 950 amino acids, about 195 amino acids to about 900 amino acids, about 195 amino acids to about 850 amino acids, about 195 amino acids to about 800 amino acids, about 195 amino acids to about 750 amino acids, about 195 amino acids to about 700 amino acids, about 195 amino acids to about 650 amino acids, about 195 amino acids to about 600 amino acids, about 195 amino acids to about 550 amino acids, about 195 amino acids to about 500 amino acids, about 195 amino acids to about 450 amino acids, about 195 amino acids to about 400 amino acids, about 195 amino acids to about 350 amino acids, about 195 amino acids to about 300 amino acids, about 195 amino acids to about 280 amino acids, about 195 amino acids to about 260 amino acids, about 195 amino acids to about 240 amino acids, about 195 amino acids to about 220 amino acids, about 195 amino acids to about 200 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 280 amino acids, about 200 amino acids to about 260 amino acids, about 200 amino acids to about 240 amino acids, about 200 amino acids to about 220 amino acids, about 220 amino acids to about 1000 amino acids, about 220 amino acids to about 950 amino acids, about 220 amino acids to about 900 amino acids, about 220 amino acids to about 850 amino acids, about 220 amino acids to about 800 amino acids, about 220 amino acids to about 750 amino acids, about 220 amino acids to about 700 amino acids, about 220 amino acids to about 650 amino acids, about 220 amino acids to about 600 amino acids, about 220 amino acids to about 550 amino acids, about 220 amino acids to about 500 amino acids, about 220 amino acids to about 450 amino acids, about 220 amino acids to about 400 amino acids, about 220 amino acids to about 350 amino acids, about 220 amino acids to about 300 amino acids, about 220 amino acids to about 280 amino acids, about 220 amino acids to about 260 amino acids, about 220 amino acids to about 240 amino acids, about 240 amino acids to about 1000 amino acids, about 240 amino acids to about 950 amino acids, about 240 amino acids to about 900 amino acids, about 240 amino acids to about 850 amino acids, about 240 amino acids to about 800 amino acids, about 240 amino acids to about 750 amino acids, about 240 amino acids to about 700 amino acids, about 240 amino acids to about 650 amino acids, about 240 amino acids to about 600 amino acids, about 240 amino acids to about 550 amino acids, about 240 amino acids to about 500 amino acids, about 240 amino acids to about 450 amino acids, about 240 amino acids to about 400 amino acids, about 240 amino acids to about 350 amino acids, about 240 amino acids to about 300 amino acids, about 240 amino acids to about 280 amino acids, about 240 amino acids to about 260 amino acids, about 260 amino acids to about 1000 amino acids, about 260 amino acids to about 950 amino acids, about 260 amino acids to about 900 amino acids, about 260 amino acids to about 850 amino acids, about 260 amino acids to about 800 amino acids, about 260 amino acids to about 750 amino acids, about 260 amino acids to about 700 amino acids, about 260 amino acids to about 650 amino acids, about 260 amino acids to about 600 amino acids, about 260 amino acids to about 550 amino acids, about 260 amino acids to about 500 amino acids, about 260 amino acids to about 450 amino acids, about 260 amino acids to about 400 amino acids, about 260 amino acids to about 350 amino acids, about 260 amino acids to about 300 amino acids, about 260 amino acids to about 280 amino acids, about 280 amino acids to about 1000 amino acids, about 280 amino acids to about 950 amino acids, about 280 amino acids to about 900 amino acids, about 280 amino acids to about 850 amino acids, about 280 amino acids to about 800 amino acids, about 280 amino acids to about 750 amino acids, about 280 amino acids to about 700 amino acids, about 280 amino acids to about 650 amino acids, about 280 amino acids to about 600 amino acids, about 280 amino acids to about 550 amino acids, about 280 amino acids to about 500 amino acids, about 280 amino acids to about 450 amino acids, about 280 amino acids to about 400 amino acids, about 280 amino acids to about 350 amino acids, about 280 amino acids to about 300 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 1000 amino acids, about 350 amino acids to about 950 amino acids, about 350 amino acids to about 900 amino acids, about 350 amino acids to about 850 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 1000 amino acids, about 450 amino acids to about 950 amino acids, about 450 amino acids to about 900 amino acids, about 450 amino acids to about 850 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 1000 amino acids, about 850 amino acids to about 950 amino acids, about 850 amino acids to about 900 amino acids, about 900 amino acids to about 1000 amino acids, about 900 amino acids to about 950 amino acids, or about 950 amino acids to about 1000 amino acids.

Any of the target-binding domains described herein can bind to its target with a dissociation equilibrium constant (K_{D}) of less than 1×10⁻⁷M, less than 1×10⁻⁸ M, less than 1×10⁻⁹ M, less than 1×10⁻¹⁰ M, less than 1×10⁻¹¹ M, less than 1×10⁻¹²M, or less than 1×10⁻¹³ M. In some embodiments, the antigen-binding protein construct provided herein can bind to an identifying antigen with a K_{D} of about 1×10⁻³ M to about 1×10⁻⁵ M, about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰ M, or about 1×10⁻⁹ M to about 1 ×10⁻¹¹ M (inclusive).

Any of the target-binding domains described herein can bind to its target with a K_{D} of between about 1 pM to about 30 nM (e.g., about 1 pM to about 25 nM, about 1 pM to about 20 nM, about 1 pM to about 15 nM, about 1 pM to about 10 nM, about 1 pM to about 5 nM, about 1 pM to about 2 nM, about 1 pM to about 1 nM, about 1 pM to about 950 pM, about 1 pM to about 900 pM, about 1 pM to about 850 pM, about 1 pM to about 800 pM, about 1 pM to about 750 pM, about 1 pM to about 700 pM, about 1 pM to about 650 pM, about 1 pM to about 600 pM, about 1 pM to about 550 pM, about 1 pM to about 500 pM, about 1 pM to about 450 pM, about 1 pM to about 400 pM, about 1 pM to about 350 pM, about 1 pM to about 300 pM, about 1 pM to about 250 pM, about 1 pM to about 200 pM, about 1 pM to about 150 pM, about 1 pM to about 100 pM, about 1 pM to about 90 pM, about 1 pM to about 80 pM, about 1 pM to about 70 pM, about 1 pM to about 60 pM, about 1 pM to about 50 pM, about 1 pM to about 40 pM, about 1 pM to about 30 pM, about 1 pM to about 20 pM, about 1 pM to about 10 pM, about 1 pM to about 5 pM, about 1 pM to about 4 pM, about 1 pM to about 3 pM, about 1 pM to about 2 pM, about 2 pM to about 30 nM, about 2 pM to about 25 nM, about 2 pM to about 20 nM, about 2 pM to about 15 nM, about 2 pM to about 10 nM, about 2 pM to about 5 nM, about 2 pM to about 2 nM, about 2 pM to about 1 nM, about 2 pM to about 950 pM, about 2 pM to about 900 pM, about 2 pM to about 850 pM, about 2 pM to about 800 pM, about 2 pM to about 750 pM, about 2 pM to about 700 pM, about 2 pM to about 650 pM, about 2 pM to about 600 pM, about 2 pM to about 550 pM, about 2 pM to about 500 pM, about 2 pM to about 450 pM, about 2 pM to about 400 pM, about 2 pM to about 350 pM, about 2 pM to about 300 pM, about 2 pM to about 250 pM, about 2 pM to about 200 pM, about 2 pM to about 150 pM, about 2 pM to about 100 pM, about 2 pM to about 90 pM, about 2 pM to about 80 pM, about 2 pM to about 70 pM, about 2 pM to about 60 pM, about 2 pM to about 50 pM, about 2 pM to about 40 pM, about 2 pM to about 30 pM, about 2 pM to about 20 pM, about 2 pM to about 10 pM, about 2 pM to about 5 pM, about 2 pM to about 4 pM, about 2 pM to about 3 pM, about 5 pM to about 30 nM, about 5 pM to about 25 nM, about 5 pM to about 20 nM, about 5 pM to about 15 nM, about 5 pM to about 10 nM, about 5 pM to about 5 nM, about 5 pM to about 2 nM, about 5 pM to about 1 nM, about 5 pM to about 950 pM, about 5 pM to about 900 pM, about 5 pM to about 850 pM, about 5 pM to about 800 pM, about 5 pM to about 750 pM, about 5 pM to about 700 pM, about 5 pM to about 650 pM, about 5 pM to about 600 pM, about 5 pM to about 550 pM, about 5 pM to about 500 pM, about 5 pM to about 450 pM, about 5 pM to about 400 pM, about 5 pM to about 350 pM, about 5 pM to about 300 pM, about 5 pM to about 250 pM, about 5 pM to about 200 pM, about 5 pM to about 150 pM, about 5 pM to about 100 pM, about 5 pM to about 90 pM, about 5 pM to about 80 pM, about 5 pM to about 70 pM, about 5 pM to about 60 pM, about 5 pM to about 50 pM, about 5 pM to about 40 pM, about 5 pM to about 30 pM, about 5 pM to about 20 pM, about 5 pM to about 10 pM, about 10 pM to about 30 nM, about 10 pM to about 25 nM, about 10 pM to about 20 nM, about 10 pM to about 15 nM, about 10 pM to about 10 nM, about 10 pM to about 5 nM, about 10 pM to about 2 nM, about 10 pM to about 1 nM, about 10 pM to about 950 pM, about 10 pM to about 900 pM, about 10 pM to about 850 pM, about 10 pM to about 800 pM, about 10 pM to about 750 pM, about 10 pM to about 700 pM, about 10 pM to about 650 pM, about 10 pM to about 600 pM, about 10 pM to about 550 pM, about 10 pM to about 500 pM, about 10 pM to about 450 pM, about 10 pM to about 400 pM, about 10 pM to about 350 pM, about 10 pM to about 300 pM, about 10 pM to about 250 pM, about 10 pM to about 200 pM, about 10 pM to about 150 pM, about 10 pM to about 100 pM, about 10 pM to about 90 pM, about 10 pM to about 80 pM, about 10 pM to about 70 pM, about 10 pM to about 60 pM, about 10 pM to about 50 pM, about 10 pM to about 40 pM, about 10 pM to about 30 pM, about 10 pM to about 20 pM, about 15 pM to about 30 nM, about 15 pM to about 25 nM, about 15 pM to about 20 nM, about 15 pM to about 15 nM, about 15 pM to about 10 nM, about 15 pM to about 5 nM, about 15 pM to about 2 nM, about 15 pM to about 1 nM, about 15 pM to about 950 pM, about 15 pM to about 900 pM, about 15 pM to about 850 pM, about 15 pM to about 800 pM, about 15 pM to about 750 pM, about 15 pM to about 700 pM, about 15 pM to about 650 pM, about 15 pM to about 600 pM, about 15 pM to about 550 pM, about 15 pM to about 500 pM, about 15 pM to about 450 pM, about 15 pM to about 400 pM, about 15 pM to about 350 pM, about 15 pM to about 300 pM, about 15 pM to about 250 pM, about 15 pM to about 200 pM, about 15 pM to about 150 pM, about 15 pM to about 100 pM, about 15 pM to about 90 pM, about 15 pM to about 80 pM, about 15 pM to about 70 pM, about 15 pM to about 60 pM, about 15 pM to about 50 pM, about 15 pM to about 40 pM, about 15 pM to about 30 pM, about 15 pM to about 20 pM, about 20 pM to about 30 nM, about 20 pM to about 25 nM, about 20 pM to about 20 nM, about 20 pM to about 15 nM, about 20 pM to about 10 nM, about 20 pM to about 5 nM, about 20 pM to about 2 nM, about 20 pM to about 1 nM, about 20 pM to about 950 pM, about 20 pM to about 900 pM, about 20 pM to about 850 pM, about 20 pM to about 800 pM, about 20 pM to about 750 pM, about 20 pM to about 700 pM, about 20 pM to about 650 pM, about 20 pM to about 600 pM, about 20 pM to about 550 pM, about 20 pM to about 500 pM, about 20 pM to about 450 pM, about 20 pM to about 400 pM, about 20 pM to about 350 pM, about 20 pM to about 300 pM, about 20 pM to about 250 pM, about 20 pM to about 20 pM, about 200 pM to about 150 pM, about 20 pM to about 100 pM, about 20 pM to about 90 pM, about 20 pM to about 80 pM, about 20 pM to about 70 pM, about 20 pM to about 60 pM, about 20 pM to about 50 pM, about 20 pM to about 40 pM, about 20 pM to about 30 pM, about 30 pM to about 30 nM, about 30 pM to about 25 nM, about 30 pM to about 30 nM, about 30 pM to about 15 nM, about 30 pM to about 10 nM, about 30 pM to about 5 nM, about 30 pM to about 2 nM, about 30 pM to about 1 nM, about 30 pM to about 950 pM, about 30 pM to about 900 pM, about 30 pM to about 850 pM, about 30 pM to about 800 pM, about 30 pM to about 750 pM, about 30 pM to about 700 pM, about 30 pM to about 650 pM, about 30 pM to about 600 pM, about 30 pM to about 550 pM, about 30 pM to about 500 pM, about 30 pM to about 450 pM, about 30 pM to about 400 pM, about 30 pM to about 350 pM, about 30 pM to about 300 pM, about 30 pM to about 250 pM, about 30 pM to about 200 pM, about 30 pM to about 150 pM, about 30 pM to about 100 pM, about 30 pM to about 90 pM, about 30 pM to about 80 pM, about 30 pM to about 70 pM, about 30 pM to about 60 pM, about 30 pM to about 50 pM, about 30 pM to about 40 pM, about 40 pM to about 30 nM, about 40 pM to about 25 nM, about 40 pM to about 30 nM, about 40 pM to about 15 nM, about 40 pM to about 10 nM, about 40 pM to about 5 nM, about 40 pM to about 2 nM, about 40 pM to about 1 nM, about 40 pM to about 950 pM, about 40 pM to about 900 pM, about 40 pM to about 850 pM, about 40 pM to about 800 pM, about 40 pM to about 750 pM, about 40 pM to about 700 pM, about 40 pM to about 650 pM, about 40 pM to about 600 pM, about 40 pM to about 550 pM, about 40 pM to about 500 pM, about 40 pM to about 450 pM, about 40 pM to about 400 pM, about 40 pM to about 350 pM, about 40 pM to about 300 pM, about 40 pM to about 250 pM, about 40 pM to about 200 pM, about 40 pM to about 150 pM, about 40 pM to about 100 pM, about 40 pM to about 90 pM, about 40 pM to about 80 pM, about 40 pM to about 70 pM, about 40 pM to about 60 pM, about 40 pM to about 50 pM, about 50 pM to about 30 nM, about 50 pM to about 25 nM, about 50 pM to about 30 nM, about 50 pM to about 15 nM, about 50 pM to about 10 nM, about 50 pM to about 5 nM, about 50 pM to about 2 nM, about 50 pM to about 1 nM, about 50 pM to about 950 pM, about 50 pM to about 900 pM, about 50 pM to about 850 pM, about 50 pM to about 800 pM, about 50 pM to about 750 pM, about 50 pM to about 700 pM, about 50 pM to about 650 pM, about 50 pM to about 600 pM, about 50 pM to about 550 pM, about 50 pM to about 500 pM, about 50 pM to about 450 pM, about 50 pM to about 400 pM, about 50 pM to about 350 pM, about 50 pM to about 300 pM, about 50 pM to about 250 pM, about 50 pM to about 200 pM, about 50 pM to about 150 pM, about 50 pM to about 100 pM, about 50 pM to about 90 pM, about 50 pM to about 80 pM, about 50 pM to about 70 pM, about 50 pM to about 60 pM, about 60 pM to about 30 nM, about 60 pM to about 25 nM, about 60 pM to about 30 nM, about 60 pM to about 15 nM, about 60 pM to about 10 nM, about 60 pM to about 5 nM, about 60 pM to about 2 nM, about 60 pM to about 1 nM, about 60 pM to about 950 pM, about 60 pM to about 900 pM, about 60 pM to about 850 pM, about 60 pM to about 800 pM, about 60 pM to about 750 pM, about 60 pM to about 700 pM, about 60 pM to about 650 pM, about 60 pM to about 600 pM, about 60 pM to about 550 pM, about 60 pM to about 500 pM, about 60 pM to about 450 pM, about 60 pM to about 400 pM, about 60 pM to about 350 pM, about 60 pM to about 300 pM, about 60 pM to about 250 pM, about 60 pM to about 200 pM, about 60 pM to about 150 pM, about 60 pM to about 100 pM, about 60 pM to about 90 pM, about 60 pM to about 80 pM, about 60 pM to about 70 pM, about 70 pM to about 30 nM, about 70 pM to about 25 nM, about 70 pM to about 30 nM, about 70 pM to about 15 nM, about 70 pM to about 10 nM, about 70 pM to about 5 nM, about 70 pM to about 2 nM, about 70 pM to about 1 nM, about 70 pM to about 950 pM, about 70 pM to about 900 pM, about 70 pM to about 850 pM, about 70 pM to about 800 pM, about 70 pM to about 750 pM, about 70 pM to about 700 pM, about 70 pM to about 650 pM, about 70 pM to about 600 pM, about 70 pM to about 550 pM, about 70 pM to about 500 pM, about 70 pM to about 450 pM, about 70 pM to about 400 pM, about 70 pM to about 350 pM, about 70 pM to about 300 pM, about 70 pM to about 250 pM, about 70 pM to about 200 pM, about 70 pM to about 150 pM, about 70 pM to about 100 pM, about 70 pM to about 90 pM, about 70 pM to about 80 pM, about 80 pM to about 30 nM, about 80 pM to about 25 nM, about 80 pM to about 30 nM, about 80 pM to about 15 nM, about 80 pM to about 10 nM, about 80 pM to about 5 nM, about 80 pM to about 2 nM, about 80 pM to about 1 nM, about 80 pM to about 950 pM, about 80 pM to about 900 pM, about 80 pM to about 850 pM, about 80 pM to about 800 pM, about 80 pM to about 750 pM, about 80 pM to about 700 pM, about 80 pM to about 650 pM, about 80 pM to about 600 pM, about 80 pM to about 550 pM, about 80 pM to about 500 pM, about 80 pM to about 450 pM, about 80 pM to about 400 pM, about 80 pM to about 350 pM, about 80 pM to about 300 pM, about 80 pM to about 250 pM, about 80 pM to about 200 pM, about 80 pM to about 150 pM, about 80 pM to about 100 pM, about 80 pM to about 90 pM, about 90 pM to about 30 nM, about 90 pM to about 25 nM, about 90 pM to about 30 nM, about 90 pM to about 15 nM, about 90 pM to about 10 nM, about 90 pM to about 5 nM, about 90 pM to about 2 nM, about 90 pM to about 1 nM, about 90 pM to about 950 pM, about 90 pM to about 900 pM, about 90 pM to about 850 pM, about 90 pM to about 800 pM, about 90 pM to about 750 pM, about 90 pM to about 700 pM, about 90 pM to about 650 pM, about 90 pM to about 600 pM, about 90 pM to about 550 pM, about 90 pM to about 500 pM, about 90 pM to about 450 pM, about 90 pM to about 400 pM, about 90 pM to about 350 pM, about 90 pM to about 300 pM, about 90 pM to about 250 pM, about 90 pM to about 200 pM, about 90 pM to about 150 pM, about 90 pM to about 100 pM, about 100 pM to about 30 nM, about 100 pM to about 25 nM, about 100 pM to about 30 nM, about 100 pM to about 15 nM, about 100 pM to about 10 nM, about 100 pM to about 5 nM, about 100 pM to about 2 nM, about 100 pM to about 1 nM, about 100 pM to about 950 pM, about 100 pM to about 900 pM, about 100 pM to about 850 pM, about 100 pM to about 800 pM, about 100 pM to about 750 pM, about 100 pM to about 700 pM, about 100 pM to about 650 pM, about 100 pM to about 600 pM, about 100 pM to about 550 pM, about 100 pM to about 500 pM, about 100 pM to about 450 pM, about 100 pM to about 400 pM, about 100 pM to about 350 pM, about 100 pM to about 300 pM, about 100 pM to about 250 pM, about 100 pM to about 200 pM, about 100 pM to about 150 pM, about 150 pM to about 30 nM, about 150 pM to about 25 nM, about 150 pM to about 30 nM, about 150 pM to about 15 nM, about 150 pM to about 10 nM, about 150 pM to about 5 nM, about 150 pM to about 2 nM, about 150 pM to about 1 nM, about 150 pM to about 950 pM, about 150 pM to about 900 pM, about 150 pM to about 850 pM, about 150 pM to about 800 pM, about 150 pM to about 750 pM, about 150 pM to about 700 pM, about 150 pM to about 650 pM, about 150 pM to about 600 pM, about 150 pM to about 550 pM, about 150 pM to about 500 pM, about 150 pM to about 450 pM, about 150 pM to about 400 pM, about 150 pM to about 350 pM, about 150 pM to about 300 pM, about 150 pM to about 250 pM, about 150 pM to about 200 pM, about 200 pM to about 30 nM, about 200 pM to about 25 nM, about 200 pM to about 30 nM, about 200 pM to about 15 nM, about 200 pM to about 10 nM, about 200 pM to about 5 nM, about 200 pM to about 2 nM, about 200 pM to about 1 nM, about 200 pM to about 950 pM, about 200 pM to about 900 pM, about 200 pM to about 850 pM, about 200 pM to about 800 pM, about 200 pM to about 750 pM, about 200 pM to about 700 pM, about 200 pM to about 650 pM, about 200 pM to about 600 pM, about 200 pM to about 550 pM, about 200 pM to about 500 pM, about 200 pM to about 450 pM, about 200 pM to about 400 pM, about 200 pM to about 350 pM, about 200 pM to about 300 pM, about 200 pM to about 250 pM, about 300 pM to about 30 nM, about 300 pM to about 25 nM, about 300 pM to about 30 nM, about 300 pM to about 15 nM, about 300 pM to about 10 nM, about 300 pM to about 5 nM, about 300 pM to about 2 nM, about 300 pM to about 1 nM, about 300 pM to about 950 pM, about 300 pM to about 900 pM, about 300 pM to about 850 pM, about 300 pM to about 800 pM, about 300 pM to about 750 pM, about 300 pM to about 700 pM, about 300 pM to about 650 pM, about 300 pM to about 600 pM, about 300 pM to about 550 pM, about 300 pM to about 500 pM, about 300 pM to about 450 pM, about 300 pM to about 400 pM, about 300 pM to about 350 pM, about 400 pM to about 30 nM, about 400 pM to about 25 nM, about 400 pM to about 30 nM, about 400 pM to about 15 nM, about 400 pM to about 10 nM, about 400 pM to about 5 nM, about 400 pM to about 2 nM, about 400 pM to about 1 nM, about 400 pM to about 950 pM, about 400 pM to about 900 pM, about 400 pM to about 850 pM, about 400 pM to about 800 pM, about 400 pM to about 750 pM, about 400 pM to about 700 pM, about 400 pM to about 650 pM, about 400 pM to about 600 pM, about 400 pM to about 550 pM, about 400 pM to about 500 pM, about 500 pM to about 30 nM, about 500 pM to about 25 nM, about 500 pM to about 30 nM, about 500 pM to about 15 nM, about 500 pM to about 10 nM, about 500 pM to about 5 nM, about 500 pM to about 2 nM, about 500 pM to about 1 nM, about 500 pM to about 950 pM, about 500 pM to about 900 pM, about 500 pM to about 850 pM, about 500 pM to about 800 pM, about 500 pM to about 750 pM, about 500 pM to about 700 pM, about 500 pM to about 650 pM, about 500 pM to about 600 pM, about 500 pM to about 550 pM, about 600 pM to about 30 nM, about 600 pM to about 25 nM, about 600 pM to about 30 nM, about 600 pM to about 15 nM, about 600 pM to about 10 nM, about 600 pM to about 5 nM, about 600 pM to about 2 nM, about 600 pM to about 1 nM, about 600 pM to about 950 pM, about 600 pM to about 900 pM, about 600 pM to about 850 pM, about 600 pM to about 800 pM, about 600 pM to about 750 pM, about 600 pM to about 700 pM, about 600 pM to about 650 pM, about 700 pM to about 30 nM, about 700 pM to about 25 nM, about 700 pM to about 30 nM, about 700 pM to about 15 nM, about 700 pM to about 10 nM, about 700 pM to about 5 nM, about 700 pM to about 2 nM, about 700 pM to about 1 nM, about 700 pM to about 950 pM, about 700 pM to about 900 pM, about 700 pM to about 850 pM, about 700 pM to about 800 pM, about 700 pM to about 750 pM, about 800 pM to about 30 nM, about 800 pM to about 25 nM, about 800 pM to about 30 nM, about 800 pM to about 15 nM, about 800 pM to about 10 nM, about 800 pM to about 5 nM, about 800 pM to about 2 nM, about 800 pM to about 1 nM, about 800 pM to about 950 pM, about 800 pM to about 900 pM, about 800 pM to about 850 pM, about 900 pM to about 30 nM, about 900 pM to about 25 nM, about 900 pM to about 30 nM, about 900 pM to about 15 nM, about 900 pM to about 10 nM, about 900 pM to about 5 nM, about 900 pM to about 2 nM, about 900 pM to about 1 nM, about 900 pM to about 950 pM, about 1 nM to about 30 nM, about 1 nM to about 25 nM, about 1 nM to about 20 nM, about 1 nM to about 15 nM, about 1 nM to about 10 nM, about 1 nM to about 5 nM, about 2 nM to about 30 nM, about 2 nM to about 25 nM, about 2 nM to about 20 nM, about 2 nM to about 15 nM, about 2 nM to about 10 nM, about 2 nM to about 5 nM, about 4 nM to about 30 nM, about 4 nM to about 25 nM, about 4 nM to about 20 nM, about 4 nM to about 15 nM, about 4 nM to about 10 nM, about 4 nM to about 5 nM, about 5 nM to about 30 nM, about 5 nM to about 25 nM, about 5 nM to about 20 nM, about 5 nM to about 15 nM, about 5 nM to about 10 nM, about 10 nM to about 30 nM, about 10 nM to about 25 nM, about 10 nM to about 20 nM, about 10 nM to about 15 nM, about 15 nM to about 30 nM, about 15 nM to about 25 nM, about 15 nM to about 20 nM, about 20 nM to about 30 nM, and about 20 nM to about 25 nM).

Any of the target-binding domains described herein can bind to its target with a K_{D} of between about 1 nM to about 10 nM (e.g., about 1 nM to about 9 nM, about 1 nM to about 8 nM, about 1 nM to about 7 nM, about 1 nM to about 6 nM, about 1 nM to about 5 nM, about 1 nM to about 4 nM, about 1 nM to about 3 nM, about 1 nM to about 2 nM, about 2 nM to about 10 nM, about 2 nM to about 9 nM, about 2 nM to about 8 nM, about 2 nM to about 7 nM, about 2 nM to about 6 nM, about 2 nM to about 5 nM, about 2 nM to about 4 nM, about 2 nM to about 3 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 3 nM to about 4 nM, about 4 nM to about 10 nM, about 4 nM to about 9 nM, about 4 nM to about 8 nM, about 4 nM to about 7 nM, about 4 nM to about 6 nM, about 4 nM to about 5 nM, about 5 nM to about 10 nM, about 5 nM to about 9 nM, about 5 nM to about 8 nM, about 5 nM to about 7 nM, about 5 nM to about 6 nM, about 6 nM to about 10 nM, about 6 nM to about 9 nM, about 6 nM to about 8 nM, about 6 nM to about 7 nM, about 7 nM to about 10 nM, about 7 nM to about 9 nM, about 7 nM to about 8 nM, about 8 nM to about 10 nM, about 8 nM to about 9 nM, and about 9 nM to about 10 nM).

A variety of different methods known in the art can be used to determine the K_{D} values of any of the antigen-binding protein constructs described herein (e.g., an electrophoretic mobility shift assay, a filter binding assay, surface plasmon resonance, and a biomolecular binding kinetics assay, etc.).

### Soluble Interleukin or Cytokine Protein

In some embodiments of any of the multi-chain chimeric polypeptides described herein, one or both of the first target-binding domain and the second target-binding domain can be a soluble interleukin protein, soluble cytokine protein, or soluble cell surface protein. In some embodiments, the soluble interleukin protein or soluble cytokine protein, is selected from the group of: IL-2, IL-3, IL-4, IL-7, IL-8, IL-10, IL-12, IL-15, IL-17, IL-18, IL-21, PDGF-DD, SCF, FLT3L. Non-limiting examples of soluble IL-7 and IL-21 are provided below.

In some embodiments, a target-binding domain can comprise a sequence that is at least 80%, 85%, 90%, 94%, 95%, 96%, 98%, or 100% identical to SEQ ID NO: 4 or 5. In some embodiments, a target-binding domain can be encoded by a sequence that is at least 80%, 85%, 90%, 94%, 95%, 96%, 98%, or 100% identical to SEQ ID NO: 6 or 23.

Human Soluble IL-7
(SEQ ID NO: 4)
DCDIEGKDGKQYESVLMVSIDQLLDSMKEIGSNCLNNEFNFFKRHIC
DANKEGMFLFRAARKLRQFLKMNSTGDFDLHLLKVSEGTTILLNCTG
QVKGRKPAALGEAQPTKSLEENKSLKEQKKLNDLCFLKRLLQEIKTC
WNKILMGTKEH
Nucleic Acid Encoding Human Soluble IL-7
(SEQ ID NO: 123)
GATTGTGATATTGAAGGTAAAGATGGCAAACAATATGAGAGTGTTCT
AATGGTCAGCATCGATCAATTATTGGACAGCATGAAAGAAATTGGTA
GCAATTGCCTGAATAATGAATTTAACTTTTTTAAAAGACATATCTGT
GATGCTAATAAGGAAGGTATGTTTTTATTCCGTGCTGCTCGCAAGTT
GAGGCAATTTCTTAAAATGAATAGCACTGGTGATTTTGATCTCCACT
TATTAAAAGTTTCAGAAGGCACAACAATACTGTTGAACTGCACTGGC
CAGGTTAAAGGAAGAAAACCAGCTGCCCTGGGTGAAGCCCAACCAAC
AAAGAGTTTGGAAGAAAATAAATCTTTAAAGGAACAGAAAAAACTGA
ATGACTTGTGTTTCCTAAAGAGACTATTACAAGAGATAAAAACTTGT
TGGAATAAAATTTTGATGGGCACTAAAGAACAC
Human Soluble IL-21
(SEQ ID NO: 5)
QGQDRHMIRMRQLIDIVDQLKNYVNDLVPEFLPAPEDVETNCEWSAF
SCFQKAQLKSANTGNNERIINVSIKKLKRKPPSTNAGRRQKHRLTCP
SCDSYEKKPPKEFLERFKSLLQKMIHQHLSSRTHGSEDS
Nucleic Acid Encoding Human Soluble IL-21
(SEQ ID NO: 6)
CAAGGTCAAGATCGCCACATGATTAGAATGCGTCAACTTATAGATAT
TGTTGATCAGCTGAAAAATTATGTGAATGACTTGGTCCCTGAATTTC
TGCCAGCTCCAGAAGATGTAGAGACAAACTGTGAGTGGTCAGCTTTT
TCCTGTTTTCAGAAGGCCCAACTAAAGTCAGCAAATACAGGAAACAA
TGAAAGGATAATCAATGTATCAATTAAAAAGCTGAAGAGGAAACCAC
CTTCCACAAATGCAGGGAGAAGACAGAAACACAGACTAACATGCCCT
TCATGTGATTCTTATGAGAAAAAACCACCCAAAGAATTCCTAGAAAG
ATTCAAATCACTTCTCCAAAAGATGATTCATCAGCATCTGTCCTCTA
GAACACACGGAAGTGAAGATTCC
Nucleic Acid Encoding Human Soluble IL-21
(SEQ ID NO: 23)
CAGGGCCAGGACAGGCACATGATCCGGATGAGGCAGCTCATCGACAT
CGTCGACCAGCTGAAGAACTACGTGAACGACCTGGTGCCCGAGTTTC
TGCCTGCCCCCGAGGACGTGGAGACCAACTGCGAGTGGTCCGCCTTC
TCCTGCTTTCAGAAGGCCCAGCTGAAGTCCGCCAACACCGGCAACAA
CGAGCGGATCATCAACGTGAGCATCAAGAAGCTGAAGCGGAAGCCTC
CCTCCACAAACGCCGGCAGGAGGCAGAAGCACAGGCTGACCTGCCCC
AGCTGTGACTCCTACGAGAAGAAGCCCCCCAAGGAGTTCCTGGAGAG
GTTCAAGTCCCTGCTGCAGAAGATGATCCATCAGCACCTGTCCTCCA
GGACCCACGGCTCCGAGGACTCC
Mouse Soluble IL-21
(SEQ ID NO: 124)
HKSSPQGPDRLLIRLRHLIDIVEQLKIYENDLDPELLSAPQDVKGHC
EHAAFACFQKAKLKPSNPGNNKTFIIDLVAQLRRRLPARRGGKKQKH
IAKCPSCDSYEKRTPKEFLERLKWLLQKMIHQHLS
Nucleic Acid Encoding Mouse Soluble IL-21
(SEQ ID NO: 125)
CATAAATCAAGCCCCCAAGGGCCAGATCGCCTCCTGATTAGACTTCG
TCACCTTATTGACATTGTTGAACAGCTGAAAATCTATGAAAATGACT
TGGATCCTGAACTTCTATCAGCTCCACAAGATGTAAAGGGGCACTGT
GAGCATGCAGCTTTTGCCTGTTTTCAGAAGGCCAAACTCAAGCCATC
AAACCCTGGAAACAATAAGACATTCATCATTGACCTCGTGGCCCAGC
TCAGGAGGAGGCTGCCTGCCAGGAGGGGAGGAAAGAAACAGAAGCAC
ATAGCTAAATGCCCTTCCTGTGATTCGTATGAGAAAAGGACACCCAA
AGAATTCCTAGAAAGACTAAAATGGCTCCTTCAAAAGATGATTCATC
AGCATCTCTCC

In some embodiments, a soluble IL-7 protein can include a sequence that is at least 70% identical (e.g., at least 75% identical, at least 80% identical, at least 85% identical, at least 90% identical, at least 95% identical, at least 99% identical, or 100% identical) to SEQ ID NO: 4.

In some embodiments, a soluble IL-21 protein can include a sequence that is at least 70% identical (e.g., at least 75% identical, at least 80% identical, at least 85% identical, at least 90% identical, at least 95% identical, at least 99% identical, or 100% identical) to SEQ ID NO: 5 or 124. In some embodiments, a soluble IL-21 protein is encoded by a nucleic acid including a sequence at least 70% identical (e.g., at least 75% identical, at least 80% identical, at least 85% identical, at least 90% identical, at least 95% identical, at least 99% identical, or 100% identical) to SEQ ID NO: 6 or 23.

Non-limiting examples of additional soluble interleukin proteins or soluble cytokine proteins are provided below.

Human Soluble IL-2
(SEQ ID NO: 24)
APTSSSTKKTQLQLEHLLLDLQMILNGINNYKNPKLTRMLTFKFYMP
KKATELKHLQCLEEELKPLEEVLNLAQSKNFHLRPRDLISNINVIVL
ELKGSETTFMCEYADETATIVEFLNRWITFCQSIISTLT
Human Soluble IL-3
(SEQ ID NO: 25)
APMTQTTPLKTSWVNCSNMIDEIITHLKQPPLPLLDENNLNGEDQDI
LMENNLRRPNLEAFNRAVKSLQNASAIESILKNLLPCLPLATAAPTR
HPIHIKDGDWNEFRRKLTFYLKTLENAQAQQTTLSLAIF
Human Soluble IL-8
(SEQ ID NO: 26)
EGAVLPRSAKELRCQCIKTYSKPFHPKFIKELRVIESGPHCANTEII
VKLSDGRELCLDPKENWVQRVVEKFLKRAENS
Human Soluble IL-10
(SEQ ID NO: 27)
SPGQGTQSENSCTHFPGNLPNMLRDLRDAFSRVKTFFQMKDQLDNLL
LKESLLEDFKGYLGCQALSEMIQFYLEEVMPQAENQDPDIKAHVNSL
GENLKTLRLRLRRCHRFLPCENKSKAVEQVKNAFNKLQEKGIYKAMS
EFDIFINYIEAYMTMKIRN
Human Soluble IL-12β (p40)
(SEQ ID NO: 28)
IWELKKDVYVVELDWYPDAPGEMVVLTCDTPEEDGITWTLDQSSEVL
GSGKTLTIQVKEFGDAGQYTCHKGGEVLSHSLLLLHKKEDGIWSTDI
LKDQKEPKNKTFLRCEAKNYSGRFTCWWLTTISTDLTFSVKSSRGSS
DPQGVTCGAATLSAERVRGDNKEYEYSVECQEDSACPAAEESLPIEV
MVDAVHKLKYENYTSSFFIRDIIKPDPPKNLQLKPLKNSRQVEVSWE
YPDTWSTPHSYFSLTFCVQVQGKSKREKKDRVFTDKTSATVICRKNA
SISVRAQDRYYSSSWSEWASVPCS
Nucleic Acid Encoding Human Soluble IL-12β (p40)
(SEQ ID NO: 29)
ATTTGGGAACTGAAGAAGGACGTCTACGTGGTCGAACTGGACTGGTA
TCCCGATGCTCCCGGCGAAATGGTGGTGCTCACTTGTGACACCCCCG
AAGAAGACGGCATCACTTGGACCCTCGATCAGAGCAGCGAGGTGCTG
GGCTCCGGAAAGACCCTCACAATCCAAGTTAAGGAGTTCGGAGACGC
TGGCCAATACACATGCCACAAGGGAGGCGAGGTGCTCAGCCATTCCT
TATTATTATTACACAAGAAGGAAGACGGAATCTGGTCCACCGACATT
TTAAAAGATCAGAAGGAGCCCAAGAATAAGACCTTTTTAAGGTGTGA
GGCCAAAAACTACAGCGGTCGTTTCACTTGTTGGTGGCTGACCACCA
TTTCCACCGATTTAACCTTCTCCGTGAAAAGCAGCCGGGGAAGCTCC
GACCCTCAAGGTGTGACATGTGGAGCCGCTACCCTCAGCGCTGAGAG
GGTTCGTGGCGATAACAAGGAATACGAGTACAGCGTGGAGTGCCAAG
AAGATAGCGCTTGTCCCGCTGCCGAAGAATCTTTACCCATTGAGGTG
ATGGTGGACGCCGTGCACAAACTCAAGTACGAGAACTACACCTCCTC
CTTCTTTATCCGGGACATCATTAAGCCCGATCCTCCTAAGAATTTAC
AGCTGAAGCCTCTCAAAAATAGCCGGCAAGTTGAGGTCTCTTGGGAA
TATCCCGACACTTGGAGCACACCCCACAGCTACTTCTCTTTAACCTT
TTGTGTGCAAGTTCAAGGTAAAAGCAAGCGGGAGAAGAAAGACCGGG
TGTTTACCGACAAAACCAGCGCCACCGTCATCTGTCGGAAGAACGCC
TCCATCAGCGTGAGGGCTCAAGATCGTTATTACTCCAGCAGCTGGTC
CGAGTGGGCCAGCGTGCCTTGTTCC
Human Soluble IL-12α (p35)
(SEQ ID NO: 30)
RNLPVATPDPGMFPCLHHSQNLLRAVSNMLQKARQTLEFYPCTSEEI
DHEDITKDKTSTVEACLPLELTKNESCLNSRETSFITNGSCLASRKT
SEMMALCLSSIYEDLKMYQVEFKTMNAKLLMDPKRQIFLDQNMLAVI
DELMQALNENSETVPQKSSLEEPDFYKTKIKLCILLHAFRIRAVTID
RVMSYLNAS
Nucleic Acid Encoding Human Soluble IL-12α (p35)
(SEQ ID NO: 31)
CGTAACCTCCCCGTGGCTACCCCCGATCCCGGAATGTTCCCTTGTTT
ACACCACAGCCAGAATTTACTGAGGGCCGTGAGCAACATGCTGCAGA
AAGCTAGGCAGACTTTAGAATTTTACCCTTGCACCAGCGAGGAGATC
GACCATGAAGATATCACCAAGGACAAGACATCCACCGTGGAGGCTTG
TTTACCTCTGGAGCTGACAAAGAACGAGTCTTGTCTCAACTCTCGTG
AAACCAGCTTCATCACAAATGGCTCTTGTTTAGCTTCCCGGAAGACC
TCCTTTATGATGGCTTTATGCCTCAGCTCCATCTACGAGGATTTAAA
GATGTACCAAGTGGAGTTCAAGACCATGAACGCCAAGCTGCTCATGG
ACCCTAAACGGCAGATCTTTTTAGACCAGAACATGCTGGCTGTGATT
GATGAGCTGATGCAAGCTTTAAACTTCAACTCCGAGACCGTCCCTCA
GAAGTCCTCCCTCGAGGAGCCCGATTTTTACAAGACAAAGATCAAAC
TGTGCATTTTACTCCACGCCTTTAGGATCCGGGCCGTGACCATTGAC
CGGGTCATGAGCTATTTAAACGCCAGC
Exemplary Human Soluble IL-12
(SEQ ID NO: 32)
IWELKKDVYVVELDWYPDAPGEMVVLTCDTPEEDGITWTLDQSSEVL
GSGKTLTIQVKEFGDAGQYTCHKGGEVLSHSLLLLHKKEDGIWSTDI
LKDQKEPKNKTFLRCEAKNYSGRFTCWWLTTISTDLTFSVKSSRGSS
DPQGVTCGAATLSAERVRGDNKEYEYSVECQEDSACPAAEESLPIEV
MVDAVHKLKYENYTSSFFIRDIIKPDPPKNLQLKPLKNSRQVEVSWE
YPDTWSTPHSYFSLTFCVQVQGKSKREKKDRVFTDKTSATVICRKNA
SISVRAQDRYYSSSWSEWASVPCSGGGGSGGGGSGGGGSRNLPVATP
DPGMFPCLHHSQNLLRAVSNMLQKARQTLEFYPCTSEEIDHEDITKD
KTSTVEACLPLELTKNESCLNSRETSFITNGSCLASRKTSEMMALCL
SSIYEDLKMYQVEFKTMNAKLLMDPKRQIFLDQNMLAVIDELMQALN
ENSETVPQKSSLEEPDFYKTKIKLCILLHAFRIRAVTIDRVMSYLNA
S
Nucleic Acid Encoding Exemplary Human Soluble
IL-12
(SEQ ID NO: 33)
ATTTGGGAACTGAAGAAGGACGTCTACGTGGTCGAACTGGACTGGTA
TCCCGATGCTCCCGGCGAAATGGTGGTGCTCACTTGTGACACCCCCG
AAGAAGACGGCATCACTTGGACCCTCGATCAGAGCAGCGAGGTGCTG
GGCTCCGGAAAGACCCTCACAATCCAAGTTAAGGAGTTCGGAGACGC
TGGCCAATACACATGCCACAAGGGAGGCGAGGTGCTCAGCCATTCCT
TATTATTATTACACAAGAAGGAAGACGGAATCTGGTCCACCGACATT
TTAAAAGATCAGAAGGAGCCCAAGAATAAGACCTTTTTAAGGTGTGA
GGCCAAAAACTACAGCGGTCGTTTCACTTGTTGGTGGCTGACCACCA
TTTCCACCGATTTAACCTTCTCCGTGAAAAGCAGCCGGGGAAGCTCC
GACCCTCAAGGTGTGACATGTGGAGCCGCTACCCTCAGCGCTGAGAG
GGTTCGTGGCGATAACAAGGAATACGAGTACAGCGTGGAGTGCCAAG
AAGATAGCGCTTGTCCCGCTGCCGAAGAATCTTTACCCATTGAGGTG
ATGGTGGACGCCGTGCACAAACTCAAGTACGAGAACTACACCTCCTC
CTTCTTTATCCGGGACATCATTAAGCCCGATCCTCCTAAGAATTTAC
AGCTGAAGCCTCTCAAAAATAGCCGGCAAGTTGAGGTCTCTTGGGAA
TATCCCGACACTTGGAGCACACCCCACAGCTACTICTCTTTAACCTT
TTGTGTGCAAGTTCAAGGTAAAAGCAAGCGGGAGAAGAAAGACCGGG
TGTTTACCGACAAAACCAGCGCCACCGTCATCTGTCGGAAGAACGCC
TCCATCAGCGTGAGGGCTCAAGATCGTTATTACTCCAGCAGCTGGTC
CGAGTGGGCCAGCGTGCCTTGTTCCGGCGGTGGAGGATCCGGAGGAG
GTGGCTCCGGCGGCGGAGGATCTCGTAACCTCCCCGTGGCTACCCCC
GATCCCGGAATGTTCCCTTGTTTACACCACAGCCAGAATTTACTGAG
GGCCGTGAGCAACATGCTGCAGAAAGCTAGGCAGACTTTAGAATTTT
ACCCTTGCACCAGCGAGGAGATCGACCATGAAGATATCACCAAGGAC
AAGACATCCACCGTGGAGGCTTGTTTACCTCTGGAGCTGACAAAGAA
CGAGTCTTGTCTCAACTCTCGTGAAACCAGCTTCATCACAAATGGCT
CTTGTTTAGCTTCCCGGAAGACCTCCTTTATGATGGCTTTATGCCTC
AGCTCCATCTACGAGGATTTAAAGATGTACCAAGTGGAGTTCAAGAC
CATGAACGCCAAGCTGCTCATGGACCCTAAACGGCAGATCTTTTTAG
ACCAGAACATGCTGGCTGTGATTGATGAGCTGATGCAAGCTTTAAAC
TTCAACTCCGAGACCGTCCCTCAGAAGTCCTCCCTCGAGGAGCCCGA
TTTTTACAAGACAAAGATCAAACTGTGCATTTTACTCCACGCCTTTA
GGATCCGGGCCGTGACCATTGACCGGGTCATGAGCTATTTAAACGCC
AGC
Human Soluble IL-15
(SEQ ID NO: 13)
NWVNVISDLKKIEDLIQSMHIDATLYTESDVHPSCKVTAMKCFLLEL
QVISLESGDASIHDTVENLIILANNSLSSNGNVTESGCKECEELEEK
NIKEFLQSFVHIVQMFINTS
Human Soluble IL-17
(SEQ ID NO: 35)
GITIPRNPGCPNSEDKNFPRTVMVNLNIHNRNTNTNPKRSSDYYNRS
TSPWNLHRNEDPERYPSVIWEAKCRHLGCINADGNVDYHMNSVPIQQ
EILVLRREPPHCPNSFRLEKILVSVGCTCVTPIVHHVA
Human Soluble IL-18
(SEQ ID NO: 36)
YFGKLESKLSVIRNLNDQVLFIDQGNRPLFEDMTDSDCRDNAPRTIF
IISMYKDSQPRGMAVTISVKCEKISTLSCENKIISFKEMNPPDNIKD
TKSDIIFFQRSVPGHDNKMQFESSSYEGYFLACEKERDLFKLILKKE
DELGDRSIMFTVQNED
Nucleic Acid Encoding Human Soluble IL-18
(SEQ ID NO: 37)
TACTTCGGCAAACTGGAATCCAAGCTGAGCGTGATCCGGAATTTAAA
CGACCAAGTTCTGTTTATCGATCAAGGTAACCGGCCTCTGTTCGAGG
ACATGACCGACTCCGATTGCCGGGACAATGCCCCCCGGACCATCTTC
ATTATCTCCATGTACAAGGACAGCCAGCCCCGGGGCATGGCTGTGAC
AATTAGCGTGAAGTGTGAGAAAATCAGCACTTTATCTTGTGAGAACA
AGATCATCTCCTTTAAGGAAATGAACCCCCCCGATAACATCAAGGAC
ACCAAGTCCGATATCATCTTCTTCCAGCGGTCCGTGCCCGGTCACGA
TAACAAGATGCAGTTCGAATCCTCCTCCTACGAGGGCTACTTTTTAG
CTTGTGAAAAGGAGAGGGATTTATTCAAGCTGATCCTCAAGAAGGAG
GACGAGCTGGGCGATCGTTCCATCATGTTCACCGTCCAAAACGAGGA
T
Human Soluble PDGF-DD
(SEQ ID NO: 38)
RDTSATPQSASIKALRNANLRRDESNHLTDLYRRDETIQVKGNGYVQ
SPREPNSYPRNLLLTWRLHSQENTRIQLVFDNQFGLEEAENDICRYD
EVEVEDISETSTIIRGRWCGHKEVPPRIKSRTNQIKITFKSDDYFVA
KPGFKIYYSLLEDFQPAAASETNWESVTSSISGVSYNSPSVTDPTLI
ADALDKKIAEFDTVEDLLKYFNPESWQEDLENMYLDTPRYRGRSYHD
RKSKVDLDRLNDDAKRYSCTPRNYSVNIREELKLANVVFFPRCLLVQ
RCGGNCGCGTVNWRSCTCNSGKTVKKYHEVLQFEPGHIKRRGRAKTM
ALVDIQLDHHERCDCICSSRPPR
Human Soluble SCF
(SEQ ID NO: 39)
EGICRNRVTNNVKDVTKLVANLPKDYMITLKYVPGMDVLPSHCWISE
MVVQLSDSLTDLLDKFSNISEGLSNYSIIDKLVNIVDDLVECVKENS
SKDLKKSFKSPEPRLFTPEEFFRIFNRSIDAFKDFVVASETSDCVVS
STLSPEKDSRVSVTKPFMLPPVAASSLRNDSSSSNRKAKNPPGDSSL
HWAAMALPALFSLIIGFAFGALYWKKRQPSLTRAVENIQINEEDNEI
SMLQEKEREFQEV
Human Soluble FLT3L
(SEQ ID NO: 40)
TQDCSFQHSPISSDFAVKIRELSDYLLQDYPVTVASNLQDEELCGGL
WRLVLAQRWMERLKTVAGSKMQGLLERVNTEIHFVTKCAFQPPPSCL
RFVQTNISRLLQETSEQLVALKPWITRQNFSRCLELQCQPDSSTLPP
PWSPRPLEATAPTAPQPPLLLLLLLPVGLLLLAAAWCLHWQRTRRRT
PRPGEQVPPVPSPQDLLLVEH
Human IL-4
(SEQ ID NO: 113)
MGLTSQLLPPLFFLLACAGNFVHGHKCDITLQEIIKTLNSLTEQKTL
CTELTVTDIFAASKNTTEKETFCRAATVLRQFYSHHEKDTRCLGATA
QQFHRHKQLIRFLKRLDRNLWGLAGLNSCPVKEANQSTLENFLERLK
TIMREKYSKCSS
Mouse IL-4
(SEQ ID NO: 34)
HIHGCDKNHLREIIGILNEVTGEGTPCTEMDVPNVLTATKNTTESEL
VCRASKVLRIFYLKHGKTPCLKKNSSVLMELQRLFRAFRCLDSSISC
TMNESKSTSLKDELESLKSIMQMDYS

Exemplary soluble cell surface proteins include soluble MICA, MICB, and a ULP16 binding protein (e.g., ULBP1, ULBP2, ULBP3, ULBP4, ULBP5, or ULBP6). Exemplary sequences for soluble MICA, MICB, ULBP1, ULBP2, ULBP3, ULBP4, ULBP5, and ULBP6 are listed below.

Human Soluble MICA
(SEQ ID NO: 41)
EPHSLRYNLTVLSWDGSVQSGFLTEVHLDGQPFLRCDRQKCRAKPQG
QWAEDVLGNKTWDRETRDLTGNGKDLRMTLAHIKDQKEGLHSLQEIR
VCEIHEDNSTRSSQHFYYDGELFLSQNLETKEWTMPQSSRAQTLAMN
VRNFLKEDAMKTKTHYHAMHADCLQELRRYLKSGVVLRRTVPPMVNV
TRSEASEGNITVTCRASGFYPWNITLSWRQDGVSLSHDTQQWGDVLP
DGNGTYQTWVATRICQGEEQRFTCYMEHSGNHSTHPVPSGKVLVLQS
HWQTFHVSAVAAAAIFVIIIFYVRCCKKKTSAAEGPELVSLQVLDQH
PVGTSDHRDATQLGFQPLMSDLGSTGSTEGA
Human Soluble MICB
(SEQ ID NO: 42)
AEPHSLRYNLMVLSQDESVQSGFLAEGHLDGQPFLRYDRQKRRAKPQ
GQWAEDVLGAKTWDTETEDLTENGQDLRRTLTHIKDQKGGLHSLQEI
RVCEIHEDSSTRGSRHFYYDGELFLSQNLETQESTVPQSSRAQTLAM
NVTNFWKEDAMKTKTHYRAMQADCLQKLQRYLKSGVAIRRTVPPMVN
VTCSEVSEGNITVTCRASSFYPRNITLTWRQDGVSLSHNTQQWGDVL
PDGNGTYQTWVATRIRQGEEQRFTCYMEHSGNHGTHPVPSGKVLVLQ
SQRTDFPYVSAAMPCFVIIIILCVPCCKKKTSAAEGPELVSLQVLDQ
HPVGTGDHRDAAQLGFQPLMSATGSTGSTEGA
Human Soluble ULBP1
(SEQ ID NO: 43)
WVDTHCLCYDFIITPKSRPEPQWCEVQGLVDERPFLHYDCVNHKAKA
FASLGKKVNVTKTWEEQTETLRDVVDELKGQLLDIQVENLIPIEPLT
LQARMSCEHEAHGHGRGSWQFLFNGQKFLLFDSNNRKWTALHPGAKK
MTEKWEKNRDVTMFFQKISLGDCKMWLEEFLMYWEQMLDPTKPPSLA
PG
Human Soluble ULBP2
(SEQ ID NO: 44)
GRADPHSLCYDITVIPKFRPGPRWCAVQGQVDEKTFLHYDCGNKTVT
PVSPLGKKLNVTTAWKAQNPVLREVVDILTEQLRDIQLENYTPKEPL
TLQARMSCEQKAEGHSSGSWQFSFDGQIFLLFDSEKRMWTTVHPGAR
KMKEKWENDKVVAMSFHYFSMGDCIGWLEDFLMGMDSTLEPSAGAPL
AMS
Human Soluble ULBP3
(SEQ ID NO: 45)
DAHSLWYNFTIIHLPRHGQQWCEVQSQVDQKNFLSYDCGSDKVLSMG
HLEEQLYATDAWGKQLEMLREVGQRLRLELADTELEDFTPSGPLTLQ
VRMSCECEADGYIRGSWQFSFDGRKFLLFDSNNRKWTVVHAGARRMK
EKWEKDSGLTTFFKMVSMRDCKSWLRDFLMHRKKRLEPTAPPTMAPG
Human Soluble ULBP4
(SEQ ID NO: 46)
HSLCFNFTIKSLSRPGQPWCEAQVFLNKNLFLQYNSDNNMVKPLGLL
GKKVYATSTWGELTQTLGEVGRDLRMLLCDIKPQIKTSDPSTLQVEM
FCQREAERCTGASWQFATNGEKSLLFDAMNMTWTVINHEASKIKETW
KKDRGLEKYFRKLSKGDCDHWLREFLGHWEAMPEPTVSPVNASDIHW
SSSSLPDRWIILGAFILLVLMGIVLICVWWQNGEWQAGLWPLRTS
Human Soluble ULBP5
(SEQ ID NO: 47)
GLADPHSLCYDITVIPKFRPGPRWCAVQGQVDEKTFLHYDCGSKTVT
PVSPLGKKLNVTTAWKAQNPVLREVVDILTEQLLDIQLENYIPKEPL
TLQARMSCEQKAEGHGSGSWQLSFDGQIFLLFDSENRMWTTVHPGAR
KMKEKWENDKDMTMSFHYISMGDCTGWLEDFLMGMDSTLEPSAGAPP
TMSSG
Human Soluble ULBP6
(SEQ ID NO: 48)
RRDDPHSLCYDITVIPKFRPGPRWCAVQGQVDEKTFLHYDCGNKTVT
PVSPLGKKLNVTMAWKAQNPVLREVVDILTEQLLDIQLENYTPKEPL
TLQARMSCEQKAEGHSSGSWQFSIDGQTFLLFDSEKRMWTTVHPGAR
KMKEKWENDKDVAMSFHYISMGDCIGWLEDFLMGMDSTLEPSAGAPL
AMSSG

In some embodiments, a soluble IL-12 protein can include a first sequence that is at least 70% identical (e.g., at least 75% identical, at least 80% identical, at least 85% identical, at least 90% identical, at least 95% identical, at least 99% identical, or 100% identical) to SEQ ID NO: 28, and a second sequence that is at least 70% identical (e.g., at least 75% identical, at least 80% identical, at least 85% identical, at least 90% identical, at least 95% identical, at least 99% identical, or 100% identical) to SEQ ID NO: 30. In some embodiments, the soluble IL-12 can further include a linker sequence (e.g., any of the exemplary linker sequences described herein or known in the art) between the first sequence and the second sequence.

In some embodiments, a soluble IL-12 protein is encoded by a first nucleic acid encoding a first sequence at least 70% identical (e.g., at least 75% identical, at least 80% identical, at least 85% identical, at least 90% identical, at least 95% identical, at least 99% identical, or 100% identical) to SEQ ID NO: 29, and a second nucleic acid sequence encoding a second sequence that is at least 70% identical (e.g., at least 75% identical, at least 80% identical, at least 85% identical, at least 90% identical, at least 95% identical, at least 99% identical, or 100% identical) to SEQ ID NO: 31. In some embodiments, the nucleic acid encoding a soluble IL-12 protein further includes a nucleic acid sequence encoding a linker sequence (e.g., any of the exemplary linker sequences described herein or known in the art) between the first nucleic acid and the second nucleic acid.

In some embodiments, a soluble IL-12 protein includes a sequence that is at least 70% identical (e.g., at least 75% identical, at least 80% identical, at least 85% identical, at least 90% identical, at least 95% identical, at least 99% identical, or 100% identical) to SEQ ID NO: 32. In some embodiments, a soluble IL-12 protein is encoded by a nucleic acid including a sequence at least 70% identical (e.g., at least 75% identical, at least 80% identical, at least 85% identical, at least 90% identical, at least 95% identical, at least 99% identical, or 100% identical) to SEQ ID NO: 33.

In some embodiments, a soluble IL-18 protein can include a sequence that is at least 70% identical (e.g., at least 75% identical, at least 80% identical, at least 85% identical, at least 90% identical, at least 95% identical, at least 99% identical, or 100% identical) to SEQ ID NO: 36. In some embodiments, a soluble IL-18 protein is encoded by a nucleic acid including a sequence at least 70% identical (e.g., at least 75% identical, at least 80% identical, at least 85% identical, at least 90% identical, at least 95% identical, at least 99% identical, or 100% identical) to SEQ ID NO: 37.

Additional examples of soluble interleukin proteins and soluble cytokine proteins are known in the art.

### Soluble Interleukin or Cytokine Receptor

In some embodiments of any of the multi-chain chimeric polypeptides described herein, one or both of the first target-binding domain and the second target-binding domain is a soluble interleukin receptor, a soluble cytokine receptor, or a soluble cell surface receptor. In some embodiments, the soluble receptor is a soluble TGF-β receptor II (TGF-βRII) (see, e.g., those described in Yung et al., Am. J. Resp. Crit. Care Med. 194(9):1140-1151, 2016), a soluble TGF-βRIII (see, e.g., those described in Heng et al., Placenta 57:320, 2017), or a soluble NKG2D (see, e.g., Cosman et al., Immunity 14(2):123-133, 2001; Costa et al., Front. Immunol., Vol. 9, Article 1150, May 29, 2018; doi: 10.3389/fimmu.2018.01150). In some embodiments, the soluble cell surface receptor is a soluble NKp30 (see, e.g., Costa et al., Front. Immunol., Vol. 9, Article 1150, May 29, 2018; doi: 10.3389/fimmu.2018.01150), a soluble NKp44 (see, e.g., those described in Costa et al., Front. Immunol., Vol. 9, Article 1150, May 29, 2018; doi: 10.3389/fimmu.2018.01150), a soluble NKp46 (see, e.g., Mandelboim et al., Nature 409:1055-1060, 2001; Costa et al., Front. Immunol., Vol. 9, Article 1150, May 29, 2018; doi: 10.3389/fimmu.2018.01150), a soluble DNAM1 (see, e.g., those described in Costa et al., Front. Immunol., Vol. 9, Article 1150, May 29, 2018; doi: 10.3389/fimmu.2018.01150), a scMHCI (see, e.g., those described in Washburn et al., PLoS One 6(3):e18439, 2011), a scMHCII (see, e.g., those described in Bishwajit et al., Cellular Immunol. 170(1):25-33, 1996), a scTCR (see, e.g., those described in Weber et al., Nature 356(6372):793-796, 1992), a soluble CD155 (see, e.g., those described in Tahara-Hanaoka et al., Int. Immunol. 16(4):533-538, 2004), or a soluble CD28 (see, e.g., Hebbar et al., Clin. Exp. Immunol. 136:388-392, 2004).

### Exemplary Soluble TGFβRII Receptor

In some embodiments, a target-binding domain can comprise a soluble TGFβRII receptor. In some embodiments, a soluble TGFβRII receptor can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 7)
IPPHVQKSVNNDMIVTDNNGAVKFPQLCKFCDVRESTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPD,

and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to:

(SEQ ID NO: 7)
IPPHVQKSVNNDMIVTDNNGAVKFPQLCKFCDVRFSTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPD.

In some embodiments, the soluble TGFβRII receptor can further include a linker sequence (e.g., any of the exemplary linker sequences described herein or known in the art) between the first sequence and the second sequence.

In some embodiments, a soluble TGFβRII receptor is encoded by a first nucleic acid encoding a first sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 8)
ATCCCGCCACACGTGCAAAAGAGCGTGAACAACGACATGATCGTTAC
GGACAACAATGGGGCCGTGAAGTTCCCTCAGTTATGCAAATTCTGTG
ACGTGAGATTCAGCACGTGCGACAATCAGAAGAGCTGCATGTCCAAT
TGCTCAATCACAAGCATCTGCGAGAAACCCCAAGAAGTTTGCGTGGC
CGTGTGGAGAAAGAATGACGAGAACATCACCCTGGAAACCGTGTGCC
ATGATCCTAAACTCCCTTACCATGACTTTATCTTGGAGGATGCTGCT
AGCCCAAAGTGCATCATGAAAGAGAAGAAGAAACCGGGGGAAACTTT
TTTCATGTGTTCATGCAGCTCTGACGAGTGTAATGATAACATTATCT
TCAGCGAAGAGTATAACACTAGCAATCCTGAT,
or
(SEQ ID NO: 49)
ATCCCCCCCCATGTGCAAAAGAGCGTGAACAACGATATGATCGTGAC
CGACAACAACGGCGCCGTGAAGTTTCCCCAGCTCTGCAAGITCTGCG
ATGTCAGGTTCAGCACCTGCGATAATCAGAAGTCCTGCATGTCCAAC
TGCACGATCACCTCCATCTGCGAGAAGCCCCAAGAAGTGTGCGTGGC
CGTGTGGCGGAAAAATGACGAGAACATCACCCTGGAGACCGTGTGTC
ACGACCCCAAGCTCCCTTATCACGACTTCATTCTGGAGGACGCTGCC
TCCCCCAAATGCATCATGAAGGAGAAGAAGAAGCCCGGAGAGACCTT
CTTTATGTGTTCCTGTAGCAGCGACGAGTGTAACGACAACATCATCT
TCAGCGAAGAGTACAACACCAGCAACCCTGAT,

and a second nucleic acid sequence encoding a second sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 50)
ATCCCCCCCCATGTGCAAAAATCAGTGAACAATGACATGATCGTGAC
CGATAATAACGGGGCGGTAAAGTTTCCTCAACTGTGTAAATTTTGCG
ACGTCAGATTCAGCACATGCGACAATCAAAAGTCCTGCATGAGCAAC
TGCAGCATCACCTCTATCTGTGAGAAACCACAAGAAGTATGTGTGGC
GGTATGGCGGAAGAACGATGAAAATATCACGCTGGAGACCGTTTGCC
ATGATCCTAAGCTGCCATATCACGATTTCATCCTGGAGGACGCCGCT
AGCCCTAAGTGTATCATGAAGGAGAAGAAGAAGCCCGGTGAGACCTT
TTTCATGTGCTCATGCAGCTCCGACGAGTGCAACGACAACATCATCT
TCAGCGAGGAGTACAATACAAGCAATCCTGAC,
or
(SEQ ID NO: 49)
ATCCCCCCCCATGTGCAAAAGAGCGTGAACAACGATATGATCGTGAC
CGACAACAACGGCGCCGTGAAGTTTCCCCAGCTCTGCAAGTTCTGCG
ATGTCAGGTTCAGCACCTGCGATAATCAGAAGTCCTGCATGTCCAAC
TGCACGATCACCTCCATCTGCGAGAAGCCCCAAGAAGTGTGCGTGGC
CGTGTGGCGGAAAAATGACGAGAACATCACCCTGGAGACCGTGTGTC
ACGACCCCAAGCTCCCTTATCACGACTTCATTCTGGAGGACGCTGCC
TCCCCCAAATGCATCATGAAGGAGAAGAAGAAGCCCGGAGAGACCTT
CTTTATGTGTTCCTGTAGCAGCGACGAGTGTAACGACAACATCATCT
TCAGCGAAGAGTACAACACCAGCAACCCTGAT.

In some embodiments, the nucleic acid encoding a soluble TGFβRII receptor further includes a nucleic acid sequence encoding a linker sequence (e.g., any of the exemplary linker sequences described herein or known in the art) between the first nucleic acid and the second nucleic acid.

In some embodiments, a soluble TGFβRII receptor includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 9)
IPPHVQKSVNNDMIVTDNNGAVKFPQLCKFCDVRFSTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPDGGGGS
IPPHVQKSVNNDMIVTDNNGAVKFPQLCKFCDVRESTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPD,
or
(SEQ ID NO: 51)
IPPHVQKSVNNDMIVTDNNGAVKFPQLCKFCDVRFSTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPDGGGGS
GGGGSGGGGSIPPHVQKSVNNDMIVTDNNGAVKFPQLCKFCDVRFST
CDNQKSCMSNCSITSICEKPQEVCVAVWRKNDENITLETVCHDPKLP
YHDFILEDAASPKCIMKEKKKPGETFFMCSCSSDECNDNIIFSEEYN
TSNPD.

In some embodiments, a soluble TGFβRII receptor is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 10)
ATCCCGCCACACGTGCAAAAGAGCGTGAACAACGACATGATCGTTAC
GGACAACAATGGGGCCGTGAAGTTCCCTCAGTTATGCAAATTCTGTG
ACGTGAGATTCAGCACGTGCGACAATCAGAAGAGCTGCATGTCCAAT
TGCTCAATCACAAGCATCTGCGAGAAACCCCAAGAAGTTTGCGTGGC
CGTGTGGAGAAAGAATGACGAGAACATCACCCTGGAAACCGTGTGCC
ATGATCCTAAACTCCCTTACCATGACTTTATCTTGGAGGATGCTGCT
AGCCCAAAGTGCATCATGAAAGAGAAGAAGAAACCGGGGGAAACTTT
TTTCATGTGTTCATGCAGCTCTGACGAGTGTAATGATAACATTATCT
TCAGCGAAGAGTATAACACTAGCAATCCTGATGGAGGTGGCGGATCT
ATCCCCCCCCATGTGCAAAAATCAGTGAACAATGACATGATCGTGAC
CGATAATAACGGGGCGGTAAAGTTTCCTCAACTGTGTAAATTTTGCG
ACGTCAGATTCAGCACATGCGACAATCAAAAGTCCTGCATGAGCAAC
TGCAGCATCACCTCTATCTGTGAGAAACCACAAGAAGTATGTGTGGC
GGTATGGCGGAAGAACGATGAAAATATCACGCTGGAGACCGTTTGCC
ATGATCCTAAGCTGCCATATCACGATTTCATCCTGGAGGACGCCGCT
AGCCCTAAGTGTATCATGAAGGAGAAGAAGAAGCCCGGTGAGACCTT
TTTCATGTGCTCATGCAGCTCCGACGAGTGCAACGACAACATCATCT
TCAGCGAGGAGTACAATACAAGCAATCCTGAC,
or
(SEQ ID NO: 52)
ATCCCCCCCCATGTGCAAAAGAGCGTGAACAACGATATGATCGTGAC
CGACAACAACGGCGCCGTGAAGTTTCCCCAGCTCTGCAAGTTCTGCG
ATGTCAGGTTCAGCACCTGCGATAATCAGAAGTCCTGCATGTCCAAC
TGCACGATCACCTCCATCTGCGAGAAGCCCCAAGAAGTGTGCGTGGC
CGTGTGGCGGAAAAATGACGAGAACATCACCCTGGAGACCGTGTGTC
ACGACCCCAAGCTCCCTTATCACGACTTCATTCTGGAGGACGCTGCC
TCCCCCAAATGCATCATGAAGGAGAAGAAGAAGCCCGGAGAGACCTT
CTTTATGTGTTCCTGTAGCAGCGACGAGTGTAACGACAACATCATCT
TCAGCGAAGAGTACAACACCAGCAACCCTGATGGAGGTGGCGGATCC
GGAGGTGGAGGTTCTGGTGGAGGTGGGAGTATTCCTCCCCACGTGCA
GAAGAGCGTGAATAATGACATGATCGTGACCGATAACAATGGCGCCG
TGAAATTTCCCCAGCTGTGCAAATTCTGCGATGTGAGGTTTTCCACC
TGCGACAACCAGAAGTCCTGTATGAGCAACTGCACAATCACCTCCAT
CTGTGAGAAGCCTCAGGAGGTGTGCGTGGCTGTCTGGCGGAAGAATG
ACGAGAATATCACCCTGGAAACCGTCTGCCACGATCCCAAGCTGCCC
TACCACGATTTCATCCTGGAAGACGCCGCCAGCCCTAAGTGCATCAT
GAAAGAGAAAAAGAAGCCTGGCGAGACCTTTTTCATGTGCTCCTGCA
GCAGCGACGAATGCAACGACAATATCATCTTTAGCGAGGAATACAAT
ACCAGCAACCCCGAC,
or
(SEQ ID NO: 114)
ATTCCTCCTCACGTGCAGAAAAGCGTGAACAACGACATGATAGTGAC
CGACAATAACGGCGCGGTCAAGTTCCCACAGCTCTGCAAATTTTGCG
ACGTGAGATTTAGTACCTGCGACAATCAGAAATCCTGCATGAGCAAT
TGTAGCATCACTAGCATCTGTGAAAAGCCCCAAGAGGTCTGCGTAGC
AGTGTGGAGAAAAAATGACGAGAACATCACACTAGAGACCGTGTGCC
ATGACCCTAAGCTGCCTTATCATGATTTCATCCTTGAAGATGCTGCT
AGCCCTAAGTGCATTATGAAAGAGAAAAAGAAGCCTGGCGAAACCTT
TTTCATGTGTAGCTGTAGTAGCGACGAGTGCAACGATAACATCATCT
TCTCGGAGGAGTATAATACATCGAATCCTGATGGCGGCGGCGGCAGC
ATCCCTCCTCATGTTCAGAAGAGCGTGAATAACGACATGATTGTGAC
GGATAACAATGGCGCCGTAAAGTTCCCTCAGCTGTGTAAGTTCTGCG
ATGTGCGATTTAGCACCTGCGACAATCAGAAGAGCTGTATGTCCAAT
TGTTCAATCACAAGCATCTGTGAGAAGCCTCAAGAGGTGTGCGTGGC
CGTTTGGAGAAAGAACGACGAGAACATTACCCTTGAGACCGTATGCC
ACGATCCTAAGCTGCCGTATCACGACTTCATACTGGAGGATGCCGCT
AGCCCTAAATGCATCATGAAAGAAAAAAAAAAGCCTGGTGAGACATT
TTTTATGTGTAGCTGCTCGTCTGACGAGTGCAATGACAATATCATTT
TCAGCGAAGAATATAACACCTCAAACCCTGAC.

Additional examples of soluble interleukin receptors and soluble cytokine receptors are known in the art.

### Antigen-Binding Domains

In some embodiments of any of the multi-chain chimeric polypeptides described herein, one or both of the first target-binding domain and the second target-binding domain is an antigen-binding domain. In some embodiments of any of the multi-chain chimeric polypeptides described herein, the first target-binding domain and the second target-binding domain are each antigen-binding domains.

In some embodiments of any of the multi-chain chimeric polypeptides described herein, the first target-binding domain and the second target-binding domain bind specifically to the same antigen. In some embodiments of these multi-chain chimeric polypeptides, the first target-binding domain and the second target-binding domain bind specifically to the same epitope. In some embodiments of these multi-chain chimeric polypeptides, the first target-binding domain and the second target-binding domain include the same amino acid sequence.

In some embodiments of any of the multi-chain chimeric polypeptides described herein, the first target-binding domain and the second target-binding domain bind specifically to different antigens.

In some embodiments of any of the antigen-binding domains described herein can bind to an antigen selected from the group consisting of: a protein, a carbohydrate, a lipid, and a combination thereof.

In some examples, an antigen-binding domain (e.g., any of the antigen-binding domains described herein) can bind specifically to any one of tissue factor (CD142) (see e.g., those described in U.S. Pat. Nos. 9,714,297; 8,951,525; 11,447,566; and 9,708,410), CD16a (see, e.g., those described in U.S. Pat. No. 9,035,026), CD28 (see, e.g., those described in U.S. Pat. No. 7,723,482), CD3 (see, e.g., those described in U.S. Pat. No. 9,226,962), CD33 (see, e.g., those described in U.S. Pat. No. 8,759,494), CD20 (see, e.g., those described in WO 2014/026054), CD19 (see, e.g., those described in U.S. Pat. No. 9,701,758), CD22 (see, e.g., those described in WO 2003/104425), CD123 (see, e.g., those described in WO 2014/130635), IL-1R (see, e.g., those described in U.S. Pat. No. 8,741,604), IL-1 (see, e.g., those described in WO 2014/095808), VEGF (see, e.g., those described in U.S. Pat. No. 9,090,684), IL-6R (see, e.g., those described in U.S. Pat. No. 7,482,436), IL-4 (see, e.g., those described in U.S. Patent Application Publication No. 2012/0171197), IL-10 (see, e.g., those described in U.S. Patent Application Publication No. 2016/0340413), PDL-1 (see, e.g., those described in Drees et al., Protein Express. Purif 94:60-66, 2014), TIGIT (see, e.g., those described in U.S. Patent Application Publication No. 2017/0198042), PD-1 (see, e.g., those described in U.S. Pat. No. 7,488,802), TIM3 (see, e.g., those described in U.S. Pat. No. 8,552,156), CTLA4 (see, e.g., those described in WO 2012/120125), ICOS (see, e.g., those described in Humbin et al., Immunity 58:1966-1983, 2025), MICA (see, e.g., those described in WO 2016/154585), MICB (see, e.g., those described in U.S. Pat. No. 8,753,640), IL-6 (see, e.g., those described in Gejima et al., Human Antibodies 11(4):121-129, 2002), IL-8 (see, e.g., those described in U.S. Pat. No. 6,117,980), TNFα (see, e.g., those described in Geng et al., Immunol. Res. 62(3):377-385, 2015), CD26a (see, e.g., those described in WO 2017/189526), CD36 (see, e.g., those described in U.S. Patent Application Publication No. 2015/0259429), ULBP2 (see, e.g., those described in U.S. Pat. No. 9,273,136), CD30 (see, e.g., those described in Homach et al., Scand. J. Immunol. 48(5):497-501, 1998), CD200 (see, e.g., those described in U.S. Pat. No. 9,085,623), IGF-1R (see, e.g., those described in U.S. Patent Application Publication No. 2017/0051063), MUC4AC (see, e.g., those described in WO 2012/170470), MUC5AC (see, e.g., those described in U.S. Pat. No. 9,238,084), Trop-2 (see, e.g., those described in WO 2013/068946), CMET (see, e.g., those described in Edwardraja et al., Biotechnol. Bioeng. 106(3):367-375, 2010), EGFR (see, e.g., those described in Akbari et al., Protein Expr. Purf. 127:8-15, 2016), HER1 (see, e.g., those described in U.S. Patent Application Publication No. 2013/0274446), HER2 (see, e.g., those described in Cao et al., Biotechnol. Lett. 37(7):1347-1354, 2015), HER3 (see, e.g., those described in U.S. Pat. No. 9,505,843), PSMA (see, e.g., those described in Parker et al., Protein Expr. Purf. 89(2):136-145, 2013), CEA (see, e.g., those described in WO 1995/015341), B7H3 (see, e.g., those described in U.S. Pat. No. 9,371,395), EPCAM (see, e.g., those described in WO 2014/159531), BCMA (see, e.g., those described in Smith et al., Mol. Ther. 26(6):1447-1456, 2018), P-cadherin (see, e.g., those described in U.S. Pat. No. 7,452,537), CEACAM5 (see, e.g., those described in U.S. Pat. No. 9,617,345), a UL16-binding protein (see, e.g., those described in WO 2017/083612), HLA-DR (see, e.g., Pistillo et al., Exp. Clin. Immunogenet. 14(2):123-130, 1997), DLL4 (see, e.g., those described in WO 2014/007513), TYRO3 (see, e.g., those described in WO 2016/166348), AXL (see, e.g., those described in WO 2012/175692), MER (see, e.g., those described in WO 2016/106221), CD122 (see, e.g., those described in U.S. Patent Application Publication No. 2016/0367664), CD155 (see, e.g., those described in WO 2017/149538), or PDGF-DD (see, e.g., those described in U.S. Pat. No. 9,441,034).

In some embodiments of any of the multi-chain chimeric polypeptides described herein, the antigen-binding domain includes or is a scFv or a single domain antibody (e.g., a VHH or a VNAR domain). In some embodiments, the antigen-binding domains present in any of the multi-chain chimeric polypeptides described herein are each independently selected from the group consisting of: a VHH domain, a VNAR domain, and a scFv.

A VHH domain is a single monomeric variable antibody domain that can be found in camelids. A VNAR domain is a single monomeric variable antibody domain that can be found in cartilaginous fish. Non-limiting aspects of VHH domains and VNAR domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments, each of the antigen-binding domains in the multi-chain chimeric polypeptides described herein are both VHH domains, or at least one antigen-binding domain is a VHH domain. In some embodiments, each of the antigen-binding domains in the multi-chain chimeric polypeptides described herein are both VNAR domains, or at least one antigen-binding domain is a VNAR domain. In some embodiments, each of the antigen-binding domains in the multi-chain chimeric polypeptides described herein are both scFv domains, or at least one antigen-binding domain is a scFv domain.

In some embodiments, any of the antigen-binding domains described herein is a BiTE, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv. DARTs are described in, e.g., Garber, Nature Reviews Drug Discovery 13:799-801, 2014. Additional examples of antigen-binding domains that can be used in any of the multi-chain chimeric polypeptide are known in the art.

In some embodiments, any of the antigen-binding domains described herein is an antigen-binding fragment of an antibody (e.g., any of the antigen-binding fragments of an antibody described herein), a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, an Intrabody, a dock and lock, a 1mmTAC, an IgG-IgG conjugate, a Cov-X-Body, and a scFv1-PEG-scFv2. See, e.g., Spiess et al., Mol. Immunol. 67:95-106, 2015, incorporated in its entirety herewith, for a description of these elements. In some embodiments, two or more polypeptides present in the multi-chain chimeric polypeptide assemble (e.g., non-covalently assemble) to form any of the antigen-binding domains described herein. In some embodiments, the two or more polypeptides present in the multi-chain chimeric polypeptide are connected with a peptide linker sequence. For example, two or more polypeptide chains connected by a linker can assemble (e.g., non-covalently assemble) to form any of the antigen-binding domains described herein.

Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

Exemplary antigen-binding domains are provided below.

### Anti-Tissue Factor Antigen-Binding Domain

In some embodiments, the first target-binding domain, the second target-binding domain, and/or one or more of the one or more additional target-binding domains can be an anti-tissue factor antigen-binding domain. In some embodiments of any of the multi-chain chimeric polypeptides described herein, the additional target-binding domain includes or is an anti-tissue factor antigen-binding domain.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 53)
DIQMTQSPASLSASVGDRVTITCLASQTIDTWLAWYLQKPGKSPQLL
IYAATNLADGVPSRFSGSGSGTDESFTISSLQPEDFATYYCQQVYSS
PFTFGQGTKLEIKGGGGSQIQLVQSGGEVKKPGASVRVSCKASGYSF
TDYNVYWVRQSPGKGLEWIGYIDPYNGITIYDQNFKGKATLTVDKST
STAYMELSSLRSEDTAVYFCARDVTTALDFWGQGTTVTVSS.

In some embodiments, an anti-tissue factor antibody can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 53)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSQIQLVQSGGEVKKPGASVRVSCKASGYSFTDYNVYWVR
QSPGKGLEWIGYIDPYNGITIYDQNFKGKATLTVDKSTSTAYMELSSLRS
EDTAVYFCARDVTTALDFWGQGTTVTVSS,

and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to:

(SEQ ID NO: 53)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSQIQLVQSGGEVKKPGASVRVSCKASGYSFTDYNVYWVR
QSPGKGLEWIGYIDPYNGITIYDQNFKGKATLTVDKSTSTAYMELSSLRS
EDTAVYFCARDVTTALDFWGQGTTVTVSS.

In some embodiments, the anti-tissue factor antigen-binding domain can further include a linker sequence (e.g., any of the exemplary linker sequences described herein or known in the art) between the first sequence and the second sequence.

In some embodiments, the first target-binding domain, the second target-binding domain, and/or one or more of the one or more additional target-binding domains can be an anti-tissue factor diabody. In some embodiments of any of the multi-chain chimeric polypeptides described herein, the additional target-binding domain includes or is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 15)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSQIQLVQSGGEVKKPGASVRVSCKASGYSFTDYNVYWVR
QSPGKGLEWIGYIDPYNGITIYDQNFKGKATLTVDKSTSTAYMELSSLRS
EDTAVYFCARDVTTALDFWGQGTTVTVSSGGGGSGGGGSGGGGSDIQMTQ
SPASLSASVGDRVTITCLASQTIDTWLAWYLQKPGKSPQLLIYAATNLAD
GVPSRFSGSGSGTDFSFTISSLQPEDFATYYCQQVYSSPFTFGQGTKLEI
KGGGGSQIQLVQSGGEVKKPGASVRVSCKASGYSFTDYNVYWVRQSPGKG
LEWIGYIDPYNGITIYDQNFKGKATLTVDKSTSTAYMELSSLRSEDTAVY
FCARDVTTALDFWGQGTTVTVSS.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 16)
GATATTCAGATGACACAGTCACCTGCCTCACTGTCAGCGTCTGTGGGGGA
CCGCGTGACAATTACGTGCCTGGCTAGCCAAACCATAGATACCTGGCTGG
CGTGGTACTTGCAGAAACCTGGAAAGTCGCCACAGCTGCTGATCTATGCG
GCAACTAACCTGGCGGACGGCGTACCCTCGCGTTTTTCCGGGTCTGGCAG
CGGTACCGATTTCTCCTTCACTATTAGCAGCCTTCAGCCGGAGGACTTTG
CTACATATTATTGTCAGCAAGTGTACTCATCCCCTTTCACCTTCGGCCAA
GGCACCAAACTAGAAATAAAAGGCGGGGGCGGTTCACAAATTCAGCTGGT
GCAAAGCGGCGGCGAAGTGAAGAAGCCTGGAGCTAGCGTGCGTGTGAGCT
GTAAGGCATCTGGCTACTCCTTCACTGATTACAACGTGTATTGGGTTAGA
CAGTCCCCTGGTAAAGGGCTTGAGTGGATTGGCTATATAGACCCTTATAA
CGGCATTACCATATACGATCAAAACTTCAAAGGTAAGGCCACCCTGACCG
TTGACAAGTCAACAAGCACCGCCTACATGGAGCTGAGCAGCCTAAGAAGC
GAGGACACCGCTGTCTATTTCTGTGCTAGAGACGTAACCACAGCCCTGGA
TTTCTGGGGCCAAGGCACAACGGTGACAGTCAGCTCGGGTGGCGGCGGTT
CGGGTGGGGGAGGAAGCGGGGGGGGGGGATCCGACATTCAAATGACGCAA
AGCCCTGCTAGCCTTTCCGCTAGCGTGGGAGACCGCGTTACGATCACATG
TCTGGCCTCTCAGACCATCGACACTTGGCTGGCCTGGTACCTACAAAAAC
CTGGCAAAAGCCCACAACTACTGATCTATGCTGCTACGAATCTGGCCGAC
GGCGTACCGTCCCGCTTCAGCGGCTCCGGTTCTGGGACAGACTTCTCTTT
CACCATTTCATCTCTGCAGCCTGAAGATTTCGCCACATATTATTGTCAAC
AAGTGTATAGCAGCCCGTTTACCTTTGGGCAAGGCACGAAGTTAGAAATC
AAGGGCGGAGGCGGATCCCAAATTCAGCTCGTGCAGAGTGGCGGGGAAGT
CAAGAAACCTGGCGCTAGCGTGAGAGTGTCTTGTAAGGCAAGTGGCTACA
GCTTTACCGACTATAATGTGTATTGGGTGAGACAATCACCCGGCAAGGGG
CTGGAGTGGATCGGCTACATCGACCCGTATAACGGCATTACGATCTACGA
CCAAAACTTTAAAGGCAAGGCCACCCTAACGGTCGACAAGTCGACCTCCA
CCGCTTATATGGAGCTTAGCTCATTGAGAAGCGAGGATACCGCGGTGTAC
TTCTGCGCTAGAGACGTGACAACCGCCCTGGACTTCTGGGGGCAAGGCAC
AACCGTCACCGTGTCGTCC.

In some embodiments, an anti-tissue factor antigen-binding domain can include a heavy chain variable sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 129)
QIQLVQSGGEVKKPGASVRVSCKASGYSFTDYNVYWVRQSPGKGLEWIGY
IDPYNGITIYDQNFKGKATLTVDKSTSTAYMELSSLRSEDTAVYFCARDV
TTALDFWGQGTTVTVSS,

and a light chain variable sequence that is at least 70% identical (e.g., at least 75% identical, at least 80% identical, at least 85% identical, at least 90% identical, at least 92% identical, at least 95% identical, at least 96% identical, at least 98% identical, or 100% identical) to:

(SEQ ID NO: 130)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIK.

In some embodiments, the first target-binding domain, the second target-binding domain, and/or one or more of the one or more additional target-binding domains can be an anti-tissue factor scFv. In some embodiments, the anti-tissue factor scFv is a humanized anti-human tissue factor scFv. In some embodiments, a humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 131)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSGGGGSGGGGSQIQLVQSGGEVKKPGASVRVSCKASGYS
FTDYNVYWVRQSPGKGLEWIGYIDPYNGITIYDQNFKGKATLTVDKSTST
AYMELSSLRSEDTAVYFCARDVTTALDFWGQGTTVTVSS.

In some embodiments, an anti-tissue factor single chain antibody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 132)
GACATACAGATGACCCAAAGCCCTGCTAGCCTGAGCGCGAGCGTGGGCGA
CAGAGTGACCATCACCTGCCTCGCCTCTCAGACGATTGACACCTGGCTGG
CTTGGTATTTGCAGAAACCTGGGAAAAGCCCTCAGCTGCTGATCTACGCA
GCCACCAATTTAGCCGATGGCGTCCCTTCTAGATTTAGCGGCTCGGGATC
CGGCACCGATTTTAGCTTTACAATTTCATCTCTACAGCCTGAGGACTTCG
CCACCTATTATTGTCAACAAGTATATAGCAGCCCTTTCACCTTCGGCCAA
GGCACCAAGTTAGAGATCAAGGGCGGTGGCGGCTCCGGGGGCGGGGGAAG
TGGAGGTGGGGGGTCACAGATTCAGCTGGTTCAAAGCGGCGGCGAGGTGA
AGAAACCTGGCGCCTCGGTGAGAGTTAGCTGCAAAGCTAGCGGATATTCG
TTTACTGACTACAACGTGTATTGGGTGAGGCAGAGCCCGGGCAAGGGACT
GGAGTGGATTGGGTACATTGACCCCTACAACGGCATAACCATATATGATC
AGAACTTCAAGGGCAAAGCCACGCTGACCGTGGATAAAAGCACAAGCACC
GCTTATATGGAGTTGAGCTCCCTCCGTAGCGAAGACACGGCAGTATACTT
TTGCGCACGGGATGTGACCACGGCCCTAGATTTCTGGGGACAAGGGACAA
CCGTTACTGTTAGTTCC.

### Anti-CD3 Antigen-Binding Domains

In some embodiments of any of the single-chain chimeric polypeptides described herein, the first target-binding domain, the second target-binding domain, and/or one or more of the one or more additional antigen-binding domains can be an anti-CD3 antigen-binding domain.

scCD3 Antibody

In some embodiments, the first target-binding domain, the second target-binding domain, and/or one or more of the one or more additional target-binding domains can be a single-chain anti-CD3 antibody. In some embodiments of any of the multi-chain chimeric polypeptides described herein, the additional target-binding domain includes or is a single-chain anti-CD3 antibody. In some embodiments, a single chain anti-CD3 (scCD3) antibody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 17)
DIQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFGQ
GTKVEIKGGGGSGGGGSGGGGSEVQLVESGGGLVQPGGSLRLSCAASGYS
FTGYTMNWVRQAPGKGLEWVALINPYKGVSTYNQKFKDRFTISVDKSKNT
AYLQMNSLRAEDTAVYYCARSGYYGDSDWYFDVWGQGTLVTVSS.

In some embodiments, a scCD3 antibody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 18)
GATATTCAGATGACGCAGTCGCCAAGCTCACTCTCGGCCTCCGTAGGAGA
CAGAGTTACCATCACCTGCAGAGCTAGCCAAGACATCAGAAACTACCTGA
ACTGGTATCAGCAGAAGCCTGGCAAGGCCCCTAAGCTCCTGATCTACTAC
ACGAGCAGACTTGAGAGCGGGGTACCATCCCGCTTCAGCGGATCTGGAAG
CGGCACAGACTACACCCTGACCATCTCTAGTCTGCAGCCTGAGGACTTCG
CCACTTACTATTGTCAGCAAGGCAACACCCTGCCTTGGACCTTTGGCCAA
GGCACCAAAGTTGAGATAAAGGGAGGGGGAGGGAGCGGTGGCGGAGGCTC
GGGTGGGGGTGGCTCAGAAGTACAGCTGGTTGAGTCCGGCGGTGGTCTGG
TGCAGCCTGGCGGAAGCCTGAGACTGAGCTGCGCCGCAAGCGGCTATAGC
TTCACTGGCTATACCATGAACTGGGTGCGCCAAGCCCCTGGCAAGGGCCT
GGAATGGGTGGCCCTGATCAACCCTTACAAGGGCGTGAGCACCTACAATC
AGAAGTTCAAGGACAGATTCACCATCAGCGTGGACAAGAGCAAGAACACC
GCCTACCTGCAGATGAACAGCCTGAGAGCCGAGGACACTGCGGTCTATTA
CTGTGCTAGAAGCGGCTACTACGGCGACAGCGACTGGTACTTCGACGTGT
GGGGGCAAGGCACCTTGGTAACGGTAAGCTCC.

In some embodiments, a scCD3 antibody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 19)
DIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSVEGGSGGSGGSGGSGGVDDIQLTQSPAIMSA
SPGEKVTMTCRASSSVSYMNWYQQKSGTSPKRWIYDTSKVASGVPYRFSG
SGSGTSYSLTISSMEAEDAATYYCQQWSSNPLTFGAGTKLELK.

In some embodiments, a scCD3 antibody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 20)
GACATCAAGCTGCAGCAGAGCGGCGCCGAGCTGGCTAGACCTGGGGCTAG
CGTCAAGATGAGCTGCAAGACAAGCGGCTACACTTTCACAAGATACACCA
TGCACTGGGTGAAGCAGAGACCTGGCCAAGGCCTGGAATGGATCGGTTAT
ATCAACCCTAGCAGAGGCTACACCAACTACAATCAGAAGTTCAAGGACAA
AGCCACCCTGACAACTGATAAGAGCAGCAGCACCGCCTATATGCAACTGT
CAAGCCTCACCTCAGAGGACAGCGCCGTGTACTACTGCGCTAGATACTAC
GACGACCACTACTGCCTGGACTATTGGGGCCAAGGCACTACTTTGACTGT
TAGCTCGGTCGAGGGAGGCTCCGGCGGGAGTGGTGGTTCTGGCGGCAGCG
GCGGAGTTGACGACATTCAGCTGACACAAAGTCCTGCCATAATGAGCGCT
AGCCCTGGCGAGAAGGTGACCATGACCTGCAGAGCTAGCAGCAGCGTGAG
CTACATGAACTGGTATCAGCAGAAGAGCGGCACAAGCCCTAAAAGATGGA
TCTACGACACAAGCAAGGTGGCTAGCGGCGTGCCTTACCGGTTTAGCGGC
TCTGGTAGCGGCACAAGCTATAGCCTCACGATTAGCTCAATGGAGGCCGA
GGACGCCGCCACGTATTATTGCCAACAGTGGAGCAGCAACCCTCTGACCT
TCGGCGCCGGGACCAAGCTGGAACTTAAG.

Anti-CD3 scFv

In some embodiments, the first target-binding domain, the second target-binding domain, and/or one or more of the one or more additional target-binding domains can be an anti-CD3 scFv. In some embodiments, the anti-CD3 scFv can include a heavy chain variable domain including a sequence that is at least 70% identical (e.g., at least 75% identical, at least 80% identical, at least 85% identical, at least 90% identical, at least 95% identical, at least 99% identical, or 100% identical) to

(SEQ ID NO: 54)
QVQLQQSGAELARPGASVKMSCKASGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSS.

and/or a light chain variable domain including a sequence that is at least 70% identical (e.g., at least 75% identical, at least 80% identical, at least 85% identical, at least 90% identical, at least 95% identical, at least 99% identical, or 100% identical) to

(SEQ ID NO: 55)
QIVLTQSPAIMSASPGEKVTMTCSASSSVSYMNWYQQKSGTSPKRWIYDT
SKLASGVPAHFRGSGSGTSYSLTISGMEAEDAATYYCQQWSSNPFTFGSG
TKLEINR.

In some embodiments, a scFv (e.g., any of the scFvs described herein) can include a linker sequence (e.g., any of the exemplary linker sequences described herein or known in the art) between the heavy chain variable domain and the light chain variable domain. In some embodiments, the anti-CD3 scFv can include a heavy chain variable domain encoded by a nucleic acid including a sequence that is at least 70% identical (e.g., at least 75% identical, at least 80% identical, at least 85% identical, at least 90% identical, at least 95% identical, at least 99% identical, or 100% identical) to

(SEQ ID NO: 56)
CAAGTTCAGCTCCAGCAAAGCGGCGCCGAACTCGCTCGGCCCGGCGCTTC
CGTGAAGATGTCTTGTAAGGCCTCCGGCTATACCTTCACCCGGTACACAA
TGCACTGGGTCAAGCAACGGCCCGGTCAAGGTTTAGAGTGGATTGGCTAT
ATCAACCCCTCCCGGGGCTATACCAACTACAACCAGAAGTTCAAGGACAA
AGCCACCCTCACCACCGACAAGTCCAGCAGCACCGCTTACATGCAGCTGA
GCTCTTTAACATCCGAGGATTCCGCCGTGTACTACTGCGCTCGGTACTAC
GACGATCATTACTGCCTCGATTACTGGGGCCAAGGTACCACCTTAACAGT
CTCCTCC,

and/or a light chain variable domain encoded by a nucleic acid including a sequence that is at least 70% identical (e.g., at least 75% identical, at least 80% identical, at least 85% identical, at least 90% identical, at least 95% identical, at least 99% identical, or 100% identical) to

(SEQ ID NO: 57)
CAGATCGTGCTGACCCAGTCCCCCGCTATTATGAGCGCTAGCCCCGGTG
AAAAGGTGACTATGACATGCAGCGCCAGCTCTTCCGTGAGCTACATGAA
CTGGTATCAGCAGAAGTCCGGCACCAGCCCTAAAAGGTGGATCTACGAC
ACCAGCAAGCTGGCCAGCGGCGTCCCCGCTCACTTTCGGGGCTCCGGCT
CCGGAACAAGCTACTCTCTGACCATCAGCGGCATGGAAGCCGAGGATGC
CGCTACCTATTACTGTCAGCAGTGGAGCTCCAACCCCTTCACCTTTGGA
TCCGGCACCAAGCTCGAGATTAATCGT.

In some embodiments, an anti-CD3 scFv can include a sequence that is at least 70% identical (e.g., at least 75% identical, at least 80% identical, at least 85% identical, at least 90% identical, at least 95% identical, at least 99% identical, or 100% identical) to

(SEQ ID NO: 58)
QIVLTQSPAIMSASPGEKVTMTCSASSSVSYMNWYQQKSGTSPKRWIYD
TSKLASGVPAHFRGSGSGTSYSLTISGMEAEDAATYYCQQWSSNPFTFG
SGTKLEINRGGGGSGGGGSGGGGSQVQLQQSGAELARPGASVKMSCKAS
GYTFTRYTMHWVKQRPGQGLEWIGYINPSRGYTNYNQKFKDKATLTTDK
SSSTAYMQLSSLTSEDSAVYYCARYYDDHYCLDYWGQGTTLTVSS.

In some embodiments, an anti-CD3 scFv can include the six CDRs present in SEQ ID NO: 58.

In some embodiments, an anti-CD3 scFv can include a sequence encoded by a nucleic acid sequence that is at least 70% identical (e.g., at least 75% identical, at least 80% identical, at least 85% identical, at least 90% identical, at least 95% identical, at least 99% identical, or 100% identical) to

(SEQ ID NO: 59)
CAGATCGTGCTGACCCAGTCCCCCGCTATTATGAGCGCTAGCCCCGGTG
AAAAGGTGACTATGACATGCAGCGCCAGCTCTTCCGTGAGCTACATGAA
CTGGTATCAGCAGAAGTCCGGCACCAGCCCTAAAAGGTGGATCTACGAC
ACCAGCAAGCTGGCCAGCGGCGTCCCCGCTCACTTTCGGGGCTCCGGCT
CCGGAACAAGCTACTCTCTGACCATCAGCGGCATGGAAGCCGAGGATGC
CGCTACCTATTACTGTCAGCAGTGGAGCTCCAACCCCTTCACCTTTGGA
TCCGGCACCAAGCTCGAGATTAATCGTGGAGGCGGAGGTAGCGGAGGAG
GCGGATCCGGCGGTGGAGGTAGCCAAGTTCAGCTCCAGCAAAGCGGCGC
CGAACTCGCTCGGCCCGGCGCTTCCGTGAAGATGTCTTGTAAGGCCTCC
GGCTATACCTTCACCCGGTACACAATGCACTGGGTCAAGCAACGGCCCG
GTCAAGGTTTAGAGTGGATTGGCTATATCAACCCCTCCCGGGGCTATAC
CAACTACAACCAGAAGTTCAAGGACAAAGCCACCCTCACCACCGACAAG
TCCAGCAGCACCGCTTACATGCAGCTGAGCTCTTTAACATCCGAGGATT
CCGCCGTGTACTACTGCGCTCGGTACTACGACGATCATTACTGCCTCGA
TTACTGGGGCCAAGGTACCACCTTAACAGTCTCCTCC.

In some embodiments, the anti-CD3 scFv can be an anti-mouse CD3 scFv. In some embodiments, an anti-mouse CD3 scFv can include a sequence that is at least 70% identical (e.g., at least 75% identical, at least 80% identical, at least 85% identical, at least 90% identical, at least 95% identical, at least 99% identical, or 100% identical) to

(SEQ ID NO: 133)
DIQMTQSPSSLPASLGDRVTINCQASQDISNYLNWYQQKPGKAPKLLIY
YTNKLADGVPSRFSGSGSGRDSSFTISSLESEDIGSYYCQQYYNYPWTF
GPGTKLEIKGGGGSGGGGSGGGGSEVQLVESGGGLVQPGKSLKLSCEAS
GFTFSGYGMHWVRQAPGRGLESVAYITSSSINIKYADAVKGRFTVSRDN
AKNLLFLQMNILKSEDTAMYYCARFDWDKNYWGQGTMVTVSS.

### Additional Anti-CD3 Antigen-Binding Domains

In some embodiments, the first target-binding domain, the second target-binding domain, and/or one or more of the one or more additional target-binding domains can be an anti-CD3 antibody. In some embodiments of any of the multi-chain chimeric polypeptides described herein, the additional target-binding domain includes or is an anti-CD3 antibody.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 60)
QVQLVQSGGGVVQPGRSLRLSCKASGYTFTRYTMHWVRQAPGKGLEWIG
YINPSRGYTNYNQKVKDRFTISRDNSKNTAFLQMDSLRPEDTGVYFCAR
YYDDHYCLDYWGQGTPVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQK
SLSLSPGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 61)
DIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFG
QGTKLQITRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC.

In some embodiments of any of the multi-chain chimeric polypeptides described herein, the additional target-binding domain includes or is an anti-CD3 antibody.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 62)
QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSAKTTAPSVYPLAPVCGGTTGSSVTLGCL
VKGYFPEPVTLTWNSGSLSSGVHTFPAVLQSDLYTLSSSVTVISSTWPS
QSITCNVAHPASSTKVDKKIEPRPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 63)
QIVLTQSPAIMSASPGEKVTMTCSASSSVSYMNWYQQKSGTSPKRWIYD
TSKLASGVPAHFRGSGSGTSYSLTISGMEAEDAATYYCQQWSSNPFTFG
SGTKLEINRADTAPTVSIFPPSSEQLTSGGASVVCFLNNFYPKDINVKW
KIDGSERQNGVLNSWTDQDSKDSTYSMSSTLTLTKDEYERHNSYTCEAT
HKTSTSPIVKSFNRNEC.

### Anti-Tissue Factor and Anti-CD3 Binding Domains

In some embodiments, the first target-binding domain, the second target-binding domain, and/or one or more of the one or more additional target-binding domains includes both an anti-tissue factor domain and an anti-CD3 binding domain.

In some embodiments, a target-binding domain that includes both an anti-tissue factor domain and an anti-CD3 binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 117)
DIQMTQSPASLSASVGDRVTITCLASQTIDTWLAWYLQKPGKSPQLLIY
AATNLADGVPSRFSGSGSGTDFSFTISSLQPEDFATYYCQQVYSSPFTF
GQGTKLEIKGGGGSGGGGSGGGGSQIQLVQSGGEVKKPGASVRVSCKAS
GYSFTDYNVYWVRQSPGKGLEWIGYIDPYNGITIYDQNFKGKATLTVDK
STSTAYMELSSLRSEDTAVYFCARDVTTALDFWGQGTTVTVSSGGGGSD
IQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFG
QGTKVEIKGGGGSGGGGSGGGGSEVQLVESGGGLVQPGGSLRLSCAASG
YSFTGYTMNWVRQAPGKGLEWVALINPYKGVSTYNQKFKDRFTISVDKS
KNTAYLQMNSLRAEDTAVYYCARSGYYGDSDWYFDVWGQGTLVTVSSGG
GGS.

In some embodiments, a target-binding domain that includes both an anti-tissue factor domain and an anti-CD3 binding domain is encoded by a nucleic acid including a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 118)
GACATACAGATGACCCAAAGCCCTGCTAGCCTGAGCGCGAGCGTGGGCG
ACAGAGTGACCATCACCTGCCTCGCCTCTCAGACGATTGACACCTGGCT
GGCTTGGTATTTGCAGAAACCTGGGAAAAGCCCTCAGCTGCTGATCTAC
GCAGCCACCAATTTAGCCGATGGCGTCCCTTCTAGATTTAGCGGCTCGG
GATCCGGCACCGATTTTAGCTTTACAATTTCATCTCTACAGCCTGAGGA
CTTCGCCACCTATTATTGTCAACAAGTATATAGCAGCCCTTTCACCTTC
GGCCAAGGCACCAAGTTAGAGATCAAGGGCGGTGGCGGCTCCGGGGGCG
GGGGAAGTGGAGGTGGGGGGTCACAGATTCAGCTGGTTCAAAGCGGCGG
CGAGGTGAAGAAACCTGGCGCCTCGGTGAGAGTTAGCTGCAAAGCTAGC
GGATATTCGTTTACTGACTACAACGTGTATTGGGTGAGGCAGAGCCCGG
GCAAGGGACTGGAGTGGATTGGGTACATTGACCCCTACAACGGCATAAC
CATATATGATCAGAACTTCAAGGGCAAAGCCACGCTGACCGTGGATAAA
AGCACAAGCACCGCTTATATGGAGTTGAGCTCCCTCCGTAGCGAAGACA
CGGCAGTATACTTTTGCGCACGGGATGTGACCACGGCCCTAGATTTCTG
GGGACAAGGGACAACCGTTACTGTTAGTTCCGGCGGTGGGGGCTCGGAT
ATTCAGATGACGCAGTCGCCAAGCTCACTCTCGGCCTCCGTAGGAGACA
GAGTTACCATCACCTGCAGAGCTAGCCAAGACATCAGAAACTACCTGAA
CTGGTATCAGCAGAAGCCTGGCAAGGCCCCTAAGCTCCTGATCTACTAC
ACGAGCAGACTTGAGAGCGGGGTACCATCCCGCTTCAGCGGATCTGGAA
GCGGCACAGACTACACCCTGACCATCTCTAGTCTGCAGCCTGAGGACTT
CGCCACTTACTATTGTCAGCAAGGCAACACCCTGCCTTGGACCTTTGGC
CAAGGCACCAAAGTTGAGATAAAGGGAGGGGGAGGGAGCGGTGGCGGAG
GCTCGGGTGGGGGTGGCTCAGAAGTACAGCTGGTTGAGTCCGGCGGTGG
TCTGGTGCAGCCTGGCGGAAGCCTGAGACTGAGCTGCGCCGCAAGCGGC
TATAGCTTCACTGGCTATACCATGAACTGGGTGCGCCAAGCCCCTGGCA
AGGGCCTGGAATGGGTGGCCCTGATCAACCCTTACAAGGGCGTGAGCAC
CTACAATCAGAAGTTCAAGGACAGATTCACCATCAGCGTGGACAAGAGC
AAGAACACCGCCTACCTGCAGATGAACAGCCTGAGAGCCGAGGACACTGC
GGTCTATTACTGTGCTAGAAGCGGCTACTACGGCGACAGCGACTGGTAC
TTCGACGTGTGGGGGCAAGGCACCTTGGTAACGGTAAGCTCCGGCGGTG
GGGGCTCG.

### Anti-PD-1 Antigen-Binding Domains

In some embodiments, the first target-binding domain, the second target-binding domain, and/or one or more of the one or more additional target-binding domains can be an anti-PD-1 antigen-binding domain. In some embodiments of any of the multi-chain chimeric polypeptides described herein, the additional target-binding domain includes or is an anti-PD-1 antigen-binding domain.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 64)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSGGGGSEIVLTQSPATLSLSPGERATLS
CRASKGVSTSGYSYLHWYQQKPGQAPRLLIYLASYLESGVPARFSGSGS
GTDFTLTISSLEPEDFAVYYCQHSRDLPLTFGGGTKVEIK.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 64)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSGGGGSEIVLTQSPATLSLSPGERATLS
CRASKGVSTSGYSYLHWYQQKPGQAPRLLIYLASYLESGVPARFSGSGS
GTDFTLTISSLEPEDFAVYYCQHSRDLPLTFGGGTKVEIK,

and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to:

(SEQ ID NO: 64)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSGGGGSEIVLTQSPATLSLSPGERATLS
CRASKGVSTSGYSYLHWYQQKPGQAPRLLIYLASYLESGVPARFSGSGS
GTDFTLTISSLEPEDFAVYYCQHSRDLPLTFGGGTKVEIK.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, the anti-PD-1 antigen-binding domain is a humanized anti-human PD-1 diabody. In some embodiments, a humanized anti-human PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 21)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSGGGGSEIVLTQSPATLSLSPGERATLS
CRASKGVSTSGYSYLHWYQQKPGQAPRLLIYLASYLESGVPARFSGSGS
GTDFTLTISSLEPEDFAVYYCQHSRDLPLTFGGGTKVEIKGGGGSGGGG
SGGGGSQVQLVQSGVEVKKPGASVKVSCKASGYTFTNYYMYWVRQAPGQ
GLEWMGGINPSNGGTNFNEKFKNRVTLTTDSSTTTAYMELKSLQFDDTA
VYYCARRDYRFDMGFDYWGQGTTVTVSSGGGGSEIVLTQSPATLSLSPG
ERATLSCRASKGVSTSGYSYLHWYQQKPGQAPRLLIYLASYLESGVPAR
FSGSGSGTDFTLTISSLEPEDFAVYYCQHSRDLPLTFGGGTKVEIK.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 22)
CAAGTGCAGCTGGTGCAGAGCGGCGTGGAGGTGAAGAAGCCGGGCGCTA
GCGTGAAGGTGAGCTGCAAGGCCTCCGGATATACCTTCACAAACTACTA
CATGTATTGGGTTAGACAAGCCCCTGGTCAAGGCCTGGAGTGGATGGGC
GGTATAAACCCTTCCAATGGAGGTACTAATTTCAACGAAAAATTCAAGA
ACCGTGTGACCCTGACCACCGACAGTAGCACCACCACTGCATACATGGA
GCTGAAGAGTCTGCAGTTCGACGATACCGCGGTCTACTACTGTGCCCGA
CGAGACTATAGATTTGACATGGGCTTCGACTACTGGGGACAAGGCACTA
CGGTGACCGTGAGCAGCGGTGGCGGCGGCTCCGAAATTGTGCTGACACA
GTCGCCTGCAACCCTGTCCTTGAGCCCTGGCGAGCGGGCAACTCTGAGC
TGCCGAGCTAGCAAAGGCGTGAGTACAAGCGGCTATAGCTACCTACATT
GGTATCAGCAAAAGCCTGGCCAAGCCCCGCGACTGCTGATCTACCTGGC
TAGCTACCTGGAGAGCGGCGTTCCTGCTAGATTCAGCGGATCTGGCTCT
GGTACTGACTTCACCTTAACTATCAGTAGCCTTGAACCCGAGGACTTCG
CTGTGTACTATTGTCAGCACAGCAGAGACCTCCCTCTGACCTTTGGAGG
TGGTACCAAGGTCGAAATCAAAGGAGGCGGAGGGTCGGGCGGTGGTGGG
TCTGGAGGTGGAGGAAGCCAAGTTCAACTCGTGCAGAGCGGTGTGGAAG
TTAAAAAGCCCGGCGCTAGCGTAAAGGTGAGCTGTAAGGCTAGCGGCTA
CACCTTCACCAATTACTACATGTACTGGGTTAGGCAAGCCCCTGGACAA
GGCTTAGAATGGATGGGTGGGATAAACCCTTCGAATGGTGGAACCAACT
TTAACGAGAAGTTCAAAAACAGAGTGACGTTGACCACCGACTCTAGCAC
CACTACCGCGTACATGGAACTGAAGAGCCTGCAGTTCGATGATACAGCG
GTGTATTATTGCGCCCGAAGAGACTACCGATTCGACATGGGGTTTGACT
ACTGGGGCCAAGGCACCACCGTCACCGTCAGCAGCGGCGGAGGCGGTAG
TGAGATTGTTCTGACCCAAAGCCCTGCCACCCTCTCGTTAAGCCCGGGG
GAGAGAGCAACCCTGAGCTGTAGAGCTAGCAAGGGCGTGAGCACCTCGG
GCTACAGTTACCTGCACTGGTACCAACAGAAGCCTGGCCAAGCCCCTCG
ATTATTGATCTACTTAGCTAGCTACTTAGAAAGCGGCGTGCCCGCTAGA
TTCAGCGGCAGCGGGAGCGGCACCGACTTTACACTGACGATAAGTAGCT
TAGAGCCTGAGGACTTTGCAGTGTATTACTGCCAACATTCCCGGGACCT
GCCGCTTACCTTCGGGGGCGGCACCAAAGTGGAGATTAAA.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 134)
QVQLQQSGAELVKPGSSVKISCKASGYTFTSHFIHWIKQQPGNGLEWIG
GIYPGDGDTEYNQQFNGKATLTADKSSSTAYMRLSSLTSEDSAVYFCAT
RVPSYWFFDFWGPGTMVTVSSGGGGSRTDVALTQTPVAQPVTLGDQASI
SCRSSQSLVHSNGRTYLEWYLQKPGQSPQLLIYKVSNRFSGVPDRFIGS
GSGSDFTLTISRVEPEDLGVYYCFQATHDPNTFGAGTKLELKGGGGSGG
GGSGGGGSQVQLQQSGAELVKPGSSVKISCKASGYTFTSHFIHWIKQQP
GNGLEWIGGIYPGDGDTEYNQQFNGKATLTADKSSSTAYMRLSSLTSED
SAVYFCATRVPSYWFFDFWGPGTMVTVSSGGGGSRTDVALTQTPVAQPV
TLGDQASISCRSSQSLVHSNGRTYLEWYLQKPGQSPQLLIYKVSNRFSG
VPDRFIGSGSGSDFTLTISRVEPEDLGVYYCFQATHDPNTFGAGTKLEL
K.

In some embodiments, the anti-PD-1 antigen-binding domain is an anti-PD-1 scFv. In some embodiments, the anti-PD-1 antigen-binding domain is an anti-human PD-1 scFv. In some embodiments, an anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 135)
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIKGGGGSGGGGSGGGGSQVQLVQSGVEVKKPGASVKVS
CKASGYTFTNYYMYWVRQAPGQGLEWMGGINPSNGGTNFNEKFKNRVTL
TTDSSTTTAYMELKSLQFDDTAVYYCARRDYRFDMGFDYWGQGTTVTVS
S.

In some embodiments, the anti-PD-1 antigen-binding domain is an anti-mouse PD-1 scFv. In some embodiments, an anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 136)
RTDVALTQTPVAQPVTLGDQASISCRSSQSLVHSNGRTYLEWYLQKPGQ
SPQLLIYKVSNRFSGVPDRFIGSGSGSDFTLTISRVEPEDLGVYYCFQA
THDPNTFGAGTKLELKGGGGSGGGGSGGGGSQVQLQQSGAELVKPGSSV
KISCKASGYTFTSHFIHWIKQQPGNGLEWIGGIYPGDGDTEYNQQFNGK
ATLTADKSSSTAYMRLSSLTSEDSAVYFCATRVPSYWFFDFWGPGTMVT
VSS.

In some embodiments, the first target-binding domain, the second target-binding domain, and/or one or more of the one or more additional target-binding domains can be an anti-PD-1 antibody. In some embodiments of any of the multi-chain chimeric polypeptides described herein, the additional target-binding domain includes or is an anti-PD-1 antibody.

In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 126)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKS
LSLSLGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 127)
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC.

In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 65)
QVQLVQSGAEVKKPGASVKVSCKASGYSFTSYWMNWVRQAPGQGLEWIG
VIHPSDSETWLDQKFKDRVTITVDKSTSTAYMELSSLRSEDTAVYYCAR
EHYGTSPFAYWGQGTLVTVSSASTKGPSVFPLAPCSRSTSESTAALGCL
VKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLS
LSLG,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 66)
EIVLTQSPATLSLSPGERATLSCRASESVDNYGMSFMNWFQQKPGQPPK
LLIHAASNQGSGVPSRFSGSGSGTDFTLTISSLEPEDFAVYFCQQSKEV
PYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC.

In some embodiments, the first target-binding domain, the second target-binding domain, and/or one or more of the one or more additional target-binding domains can be an anti-PD-1 antigen-binding domain. In some embodiments of any of the multi-chain chimeric polypeptides described herein, the additional target-binding domain includes or is an anti-PD-1 antibody. In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 67)
QVQLVESGGGVVQPGRSLRLDCKASGITFSNSGMHWVRQAPGKGLEWVA
VIWYDGSKRYYADSVKGRFTISRDNSKNTLFLQMNSLRAEDTAVYYCAT
NDDYWGQGTLVTVSSASTKGPSVFPLAPCSRSTSESTAALGCLVKDYFP
EPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 68)
EIVLTQSPATLSLSPGERATLSCRASQSVSSYLAWYQQKPGQAPRLLIY
DASNRATGIPARFSGSGSGTDFTLTISSLEPEDFAVYYCQQSSNWPRTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, the first target-binding domain, the second target-binding domain, and/or one or more of the one or more additional target-binding domains can be an anti-PD-1 antigen-binding domain. In some embodiments of any of the multi-chain chimeric polypeptides described herein, the additional target-binding domain includes or is an anti-PD-1 antibody. In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 69)
EVQLLESGGGLVQPGGSLRLSCAASGFTFSSYDMSWVRQAPGKGLEWVS
TISGGGSYTYYQDSVKGRFTISRDNSKNTLYLQMNSLRAEDTAVYYCAS
PYYAMDYWGQGTTVTVSSASTKGPSVFPLAPCSRSTSESTAALGCLVKD
YFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSL
GK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 70)
DIQLTQSPSFLSAYVGDRVTITCKASQDVGTAVAWYQQKPGKAPKLLIY
WASTLHTGVPSRFSGSGSGTEFTLTISSLQPEDFATYYCQHYSSYPWTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, the first target-binding domain, the second target-binding domain, and/or one or more of the one or more additional target-binding domains can be an anti-PD-1 antigen-binding domain. In some embodiments of any of the multi-chain chimeric polypeptides described herein, the additional target-binding domain includes or is an anti-PD-1 antibody. In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 71)
EVQLLESGGVLVQPGGSLRLSCAASGFTFSNFGMTWVRQAPGKGLEWVS
GISGGGRDTYFADSVKGRFTISRDNSKNTLYLQMNSLKGEDTAVYYCVK
WGNIYFDYWGQGTLVTVSSASTKGPSVFPLAPCSRSTSESTAALGCLVK
DYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLS
LGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 72)
DIQMTQSPSSLSASVGDSITITCRASLSINTFLNWYQQKPGKAPNLLIY
AASSLHGGVPSRFSGSGSGTDFTLTIRTLQPEDFATYYCQQSSNTPFTF
GPGTVVDFRRTVAAPSVFIFPPSDEQLKSGTASVVCLLNNFYPREAKVQ
WKVDNALQSGNSQESV.

In some embodiments, the first target-binding domain, the second target-binding domain, and/or one or more of the one or more additional target-binding domains can be an anti-PD-1 antigen-binding domain. In some embodiments of any of the multi-chain chimeric polypeptides described herein, the additional target-binding domain includes or is an anti-PD-1 antibody. In some embodiments, the anti-PD-1 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 73)
QGQLVQSGAEVKKPGASVKVSCKASGYTFTDYEMHWVRQAPIHGLEWIG
VIESETGGTAYNQKFKGRVTITADKSTSTAYMELSSLRSEDTAVYYCAR
EGITTVATTYYWYFDVWGQGTTVTVSSASTKGPSVFPLAPCSRSTSEST
AALGCLVKDYFPEPVTVSWNSGALTSGVHTFPAVLQSSGLYSLSSVVTV
PSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHY
TQKSLSLSLGK,

and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 74)
DVVMTQSPLSLPVTLGQPASISCRSSQSIVHSNGNTYLEWYLQKPGQSP
QLLIYKVSNRFSGVPDRFSGSGSGTDFTLKISRVEAEDVGVYYCFQGSH
VPLTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

### Anti-CD16a Antigen-Binding Domains

In some embodiments, the first target-binding domain, the second target-binding domain, and/or one or more of the one or more additional target-binding domains can be an anti-CD16a antigen-binding domain. In some embodiments of any of the multi-chain chimeric polypeptides described herein, the additional target-binding domain includes or is an anti-CD16a antigen-binding domain.

In some embodiments, the anti-CD16a antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 119)
SELTQDPAVSVALGQTVRITCQGDSLRSYYASWYQQKPGQAPVLVIYGK
NNRPSGIPDRFSGSSSGNTASLTITGAQAEDEADYYCNSRDSSGNHVVF
GGGTKLTVGHGGGGSGGGGSGGGGSEVQLVESGGGVVRPGGSLRLSCAA
SGFTFDDYGMSWVRQAPGKGLEWVSGINWNGGSTGYADSVKGRFTISRD
NAKNSLYLQMNSLRAEDTAVYYCARGRSLLFDYWGQGTLVTVSR.

In some embodiments, the anti-CD16a antigen-binding domain is encoded by a nucleic acid that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 120)
AGCGAGCTGACCCAAGACCCTGCCGTGAGCGTGGCCCTGGGACAGACCG
TGAGAATCACCTGCCAAGGCGACAGCCTGAGAAGCTACTACGCTAGCTG
GTATCAGCAGAAGCCTGGCCAAGCCCCTGTGCTGGTGATCTACGGCAAG
AACAACAGACCTAGCGGCATCCCTGACAGATTCAGCGGCAGCAGCAGCG
GCAACACCGCTAGCCTGACCATCACCGGCGCCCAAGCCGAGGACGAGGC
CGACTACTACTGCAACAGCAGAGACAGCAGCGGCAACCACGTGGTCTTC
GGCGGAGGAACCAAACTCACCGTCGGCCACGGCGGAGGCGGAAGCGGCG
GCGGAGGCTCCGGCGGAGGCGGCAGCGAAGTGCAGCTGGTGGAGAGCGG
AGGAGGCGTCGTCAGACCTGGCGGCAGCCTGAGACTGAGCTGCGCCGCT
AGCGGCTTCACCTTCGACGACTACGGCATGAGCTGGGTGAGACAAGCCC
CTGGCAAGGGCCTGGAGTGGGTGAGCGGCATCAACTGGAACGGCGGCAG
CACCGGCTACGCCGACAGCGTGAAGGGCAGATTCACCATCAGCAGAGAC
AACGCCAAGAACAGCCTGTACCTGCAGATGAACAGCCTGAGAGCCGAGG
ACACCGCCGTGTACTACTGCGCTAGAGGCAGAAGCCTGCTGTTCGACTA
CTGGGGCCAAGGCACCCTGGTGACCGTGAGCAGA.

### Anti-HIV Antigen-Binding Domains

In some embodiments, the first target-binding domain, the second target-binding domain, and/or one or more of the one or more additional target-binding domains can be an anti-HIV antigen-binding domain. In some embodiments of any of the multi-chain chimeric polypeptides described herein, the additional target-binding domain includes or is an anti-HIV antigen-binding domain.

In some embodiments, the anti-HIV antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 121)
SYVRPLSVALGETARISCGRQALGSRAVQWYQHRPGQAPILLIYNNQDR
PSGIPERFSGTPDINFGTRATLTISGVEAGDEADYYCHMWDSRSGFSWS
FGGATRLTVLGGGGSGGGGSGGGGSQVQLQESGPGLVKPSETLSVTCSV
SGDSMNNYYWTWIRQSPGKGLEWIGYISDRESATYNPSLNSRVVISRDT
SKNQLSLKLNSVTPADTAVYYCATARRGQRIYGVVSFGEFFYYYSMDVW
GKGTTVTVSS.

In some embodiments, the anti-HIV antigen-binding domain is encoded by a nucleic acid that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 122)
TCTTATGTAAGACCGCTCTCCGTCGCATTGGGGGAGACCGCGCGCATTA
GTTGTGGACGACAAGCACTTGGATCTCGAGCAGTTCAGTGGTACCAGCA
CCGACCCGGACAAGCTCCTATTCTGCTCATTTATAATAACCAAGACAGA
CCATCAGGCATCCCCGAGCGATTCTCTGGGACCCCGGATATCAATTTCG
GAACCAGGGCGACACTTACTATTTCAGGTGTTGAGGCCGGGGACGAGGC
AGACTATTACTGCCACATGTGGGACAGCCGATCTGGATTTTCTTGGTCA
TTTGGAGGCGCAACACGCTTGACCGTTCTCGGAGGGGGTGGCTCAGGAG
GCGGCGGATCAGGAGGGGGAGGTTCACAAGTGCAACTTCAGGAATCCGG
ACCTGGACTCGTGAAGCCTTCAGAAACCCTTTCAGTAACGTGCAGCGTG
AGCGGAGATAGCATGAACAACTATTACTGGACTTGGATAAGACAATCTC
CGGGCAAGGGGCTCGAGTGGATAGGGTATATCTCAGATAGAGAATCCGC
CACTTATAATCCTAGCCTTAACAGCCGGGTAGTCATAAGCAGAGACACA
AGCAAAAATCAACTCAGTTTGAAATTGAATTCAGTAACACCTGCCGACA
CCGCAGTGTACTATTGCGCGACAGCCCGGCGAGGGCAGAGGATTTATGG
CGTCGTTAGCTTTGGAGAATTTTTCTACTATTACTCCATGGATGTGTGG
GGCAAGGGGACCACGGTTACTGTGTCTTCA.

Additional examples and aspects of antigen-binding domains are known in the art.

### Additional Target-Binding Domains

In some embodiments of any of the multi-chain chimeric polypeptides, the first chimeric polypeptide further includes one or more (e.g., two, three, four, five, six, seven, eight, nine, or ten) additional target-binding domain(s) (e.g., any of the exemplary target-binding domains described herein or known in the art), where at least one of the one or more additional target-binding domain(s) is positioned between the soluble TRβC domain (e.g., any of the exemplary soluble TRβC domains described herein or known in the art) and the first domain of the pair of affinity domains (e.g., any of the exemplary first domains of any of the exemplary pairs of affinity domains described herein). In some embodiments, the first chimeric polypeptide can further include a linker sequence (e.g., any of the exemplary linker sequences described herein or known in the art) between the soluble TRβC domain (e.g., any of the exemplary soluble TRβC domains described herein) and the at least one of the one or more additional target-binding domain(s) (e.g., any of the exemplary target-binding domains described herein or known in the art), and/or a linker sequence (e.g., any of the exemplary linker sequences described herein or known in the art) between the at least one of the one or more additional target-binding domain(s) (e.g., any of the exemplary target-binding domains described herein or known in the art) and the first domain of the pair of affinity domains (e.g., any of the exemplary first domains described herein of any of the exemplary pairs of affinity domains described herein).

In some embodiments of any of the multi-chain chimeric polypeptides described herein, the first chimeric polypeptide further includes one or more (e.g., two, three, four, five, six, seven, eight, nine, or ten) additional target-binding domains at the N-terminal and/or C-terminal end of the first chimeric polypeptide. In some embodiments, at least one of the one or more additional target-binding domains (e.g., any of the exemplary target-binding domains described herein or known in the art) directly abuts the first domain of the pair of affinity domains (e.g., any of the exemplary first domains described herein of any of the exemplary pairs of affinity domains described herein) in the first chimeric polypeptide. In some embodiments, the first chimeric polypeptide further includes a linker sequence (e.g., any of the exemplary linker sequences described herein or known in the art) between the at least one of the one or more additional target-binding domains (e.g., any of the exemplary target-binding domains described herein or known in the art) and the first domain of the pair of affinity domains (e.g., any of the exemplary first domains described herein of any of the exemplary pairs of affinity domains described herein). In some embodiments, the at least one of the one or more additional target-binding domains (e.g., any of the exemplary target-binding domains described herein or known in the art) directly abuts the first target-binding domain (e.g., any of the exemplary target-binding domains described herein or known in the art) in the first chimeric polypeptide. In some embodiments, the first chimeric polypeptide further comprises a linker sequence (e.g., any of the exemplary linker sequences described herein or known in the art) between the at least one of the one or more additional target-binding domains (e.g., any of the exemplary target-binding domains described herein or known in the art) and the first target-binding domain (e.g., any of the exemplary target-binding domains described herein or known in the art).

In some embodiments of any of the multi-chain chimeric polypeptides described herein, at least one of the one or more additional target-binding domains (e.g., any of the exemplary target-binding domains described herein or known in the art) is disposed at the N- and/or C-terminus of the first chimeric polypeptide, and at least one of the one or more additional target-binding domains (e.g., any of the exemplary target-binding domains described herein or known in the art) is positioned between the soluble TRβC domain (e.g., any of the exemplary soluble TRβC domains described herein or known in the art) and the first domain of the pair of affinity domains (e.g., any of the exemplary first domains of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the N-terminus directly abuts the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the first chimeric polypeptide further comprises a linker sequence (e.g., any of the linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the C-terminus directly abuts the first target-binding domain (e.g., any of the exemplary target-binding domains described herein or known in the art) or the first domain of the pair of affinity domains (e.g., any of the exemplary first domains of any of the exemplary pairs of affinity domains described herein) in the first chimeric polypeptide. In some embodiments, the first chimeric polypeptide further includes a linker sequence (e.g., any of the exemplary linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one of the one or more additional target-binding domains (e.g., any of the exemplary target-binding domains described herein or known in the art) is positioned between the soluble TRβC domain (e.g., any of the exemplary soluble TRβC domains described herein) and the first domain of the pair of affinity domains (e.g., any of the first domains described herein or any of the exemplary pairs of affinity domains described herein), directly abuts the soluble TRβC domain and/or the first domain of the pair of affinity domains. In some embodiments, the first chimeric polypeptide further comprises a linker sequence (e.g., any of the exemplary linker sequences described herein or known in the art) disposed (i) between the soluble TRβC domain (e.g., any of the exemplary soluble TRβC domains described herein) and the at least one of the one or more additional target-binding domains (e.g., any of the exemplary target-binding domains described herein or known in the art) positioned between the soluble TRβC domain (e.g., any of the exemplary soluble TRβC domains described herein) and the first domain of the pair of affinity domains (e.g., any of the exemplary first domains of any of the exemplary pairs of affinity domains described herein), and/or (ii) between the first domain of the pair of affinity domains and the at least one of the one or more additional target-binding domains positioned between the soluble TRβC domain and the first domain of the pair of affinity domains.

In some embodiments of any of the multi-chain chimeric polypeptides described herein, the second chimeric polypeptide further includes one or more (e.g., two, three, four, five, six, seven, eight, nine, or ten) additional target-binding domains (e.g., any of the exemplary target-binding domains described herein or known in the art) at the N-terminal end and/or the C-terminal end of the second chimeric polypeptide. In some embodiments, at least one of the one or more additional target-binding domains (e.g., any of the exemplary target-binding domains described herein or known in the art) directly abuts the second domain of the pair of affinity domains (e.g., any of the exemplary second domains of any of the exemplary pairs of affinity domains described herein) in the second chimeric polypeptide. In some embodiments, the second chimeric polypeptide further includes a linker sequence (e.g., any of the exemplary linker sequences described herein or known in the art) between at least one of the one or more additional target-binding domains (e.g., any of the exemplary target-binding domains described herein or known in the art) and the second domain of the pair of affinity domains (e.g., any of the second domains described herein of any of the exemplary pairs of affinity domains described herein) in the second chimeric polypeptide. In some embodiments, at least one of the one or more additional target-binding domains (e.g., any of the exemplary target-binding domains described herein or known in the art) directly abuts the second target-binding domain (e.g., any of the target-binding domains described herein or known in the art) in the second chimeric polypeptide. In some embodiments, the second chimeric polypeptide further includes a linker sequence (e.g., any of the exemplary linker sequences described herein or known in the art) between at least one of the one or more additional target-binding domains (e.g., any of the exemplary target-binding domains described herein or known in the art) and the second target-binding domain (e.g., any of the exemplary target-binding domains described herein or known in the art) in the second chimeric polypeptide.

In some embodiments of any of the multi-chain chimeric polypeptides described herein, two or more (e.g., three or more, four or more, five or more, six or more, seven or more, eight or more, nine or more, or ten or more) of the first target-binding domain, the second target-binding domain, and the one or more additional target-binding domains bind specifically to the same antigen. In some embodiments, two or more (e.g., three or more, four or more, five or more, six or more, seven or more, eight or more, nine or more, or ten or more) of the first target-binding domain, the second target-binding domain, and the one or more additional target-binding domains bind specifically to the same epitope. In some embodiments, two or more (e.g., three or more, four or more, five or more, six or more, seven or more, eight or more, nine or more, or ten or more) of the first target-binding domain, the second target-binding domain, and the one or more additional target-binding domains include the same amino acid sequence. In some embodiments, the first target-binding domain, the second target-binding domain, and the one or more additional target-binding domains each bind specifically to the same antigen. In some embodiments, the first target-binding domain, the second target-binding domain, and the one or more additional target-binding domains each bind specifically to the same epitope. In some embodiments, the first target-binding domain, the second target-binding domain, and the one or more additional target-binding domains each include the same amino acid sequence.

In some embodiments of any of the multi-chain chimeric polypeptides described herein, the first target-binding domain, the second target-binding domain, and the one or more additional target-binding domains bind specifically to different antigens. In some embodiments of any of the multi-chain chimeric polypeptides described herein, one or more (e.g., two or more, three or more, four or more, five or more, six or more, seven or more, eight or more, nine or more, or ten or more) of the first target-binding domain, the second target-binding domain, and the one or more target-binding domains is an antigen-binding domain. In some embodiments, the first target-binding domain, the second target-binding domain, and the one or more additional target-binding domains are each an antigen-binding domain (e.g., a scFv or a single-domain antibody).

In some embodiments of any of the multi-chain chimeric polypeptides described herein, one or more (e.g., two, three, four, five, six, seven, eight, nine, or ten) of the first target-binding domain (e.g., any of the exemplary target-binding domains described herein or known in the art), the second target-binding domain (e.g., any of the exemplary target-binding domains described herein or known in the art), and the one or more additional target-binding domains (e.g., any of the exemplary target-binding domains described herein or known in the art) bind specifically to a target independently selected from the group consisting of: DDL-3, GPC3, CD73, CECAM5/6, tissue factor, CD16a, CD28, CD3, CD33, CD20, CD19, CD22, CD123, IL-1R, IL-1, VEGF, IL-6R, IL-4, IL-10, PDL-1, TIGIT, PD-1, TIM3, CTLA4, MICA, MICB, IL-6, IL-8, TNFα, CD26a, CD36, ULBP2, CD30, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5, uPAR, a UL16-binding protein, HLA-DR, DLL4, TYRO3, AXL, MER, CD122, CD155, PDGF-DD, a ligand of TGF-β receptor II (TGF-βRII), a ligand of TGF-βRIII, a ligand of DNAM1, a ligand of NKp46, a ligand of NKp44, a ligand of NKG2D, a ligand of NKp30, a ligand for a scMHCI, a ligand for a scMHCII, a ligand for a scTCR, a ligand of ICOS, a receptor for IL-1, a receptor for IL-2, a receptor for IL-3, a receptor for IL-4, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-DD, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28.

In some embodiments of any of the multi-chain chimeric polypeptides described herein, one or more of the first target-binding domain (e.g., any of the exemplary target-binding domains described herein or known in the art), the second target-binding domain (e.g., any of the exemplary target-binding domains described herein or known in the art), and the one or more additional target-binding domains (e.g., any of the exemplary target-binding domains described herein or known in the art) is a soluble interleukin protein, a soluble cytokine protein, or a soluble cell surface protein. Non-limiting examples of soluble interleukin proteins and soluble cytokine proteins include: IL-1, IL-2, IL-3, IL-4, IL-7, IL-8, IL-10, IL-12, IL-15, IL-17, IL-18, IL-21, PDGF-DD, SCF, and FLT3L.

In some embodiments of any of the multi-chain chimeric polypeptides described herein, one or more of the first target-binding domain (e.g., any of the exemplary target-binding domains described herein or known in the art), the second target-binding domain (e.g., any of the exemplary target-binding domains described herein or known in the art), and the one or more additional target-binding domains (e.g., any of the exemplary target-binding domains described herein or known in the art) is a soluble interleukin receptor, a soluble cytokine receptor, or a soluble cell surface receptor. Non-limiting examples of soluble interleukin receptors and soluble cytokine receptors include: a soluble TGF-β receptor II (TGF-βRII) and a soluble TGF-βRIII. Non-limiting examples of soluble cell surface receptors include: a soluble NKG2D, a soluble NK30, a soluble NKp44, a soluble NKp46, a soluble DNAM1, a scMHCI, a scMHCII, a scTCR, a soluble CD155, a soluble CD122, a soluble CD3, and a soluble CD28.

### Pairs of Affinity Domains

In some embodiments, a multi-chain chimeric polypeptide includes: 1) a first chimeric polypeptide that includes a first domain of a pair of affinity domains, and 2) a second chimeric polypeptide that includes a second domain of a pair of affinity domains such that the first chimeric polypeptide and the second chimeric polypeptide associate through the binding of the first domain and the second domain of the pair of affinity domains. In some embodiments, the pair of affinity domains is a sushi domain from an alpha chain of human IL-15 receptor (IL-15Rα) and a soluble IL-15. A sushi domain, also known as a short consensus repeat or type 1 glycoprotein motif, is a common motif in protein-protein interaction. Sushi domains have been identified on a number of protein-binding molecules, including complement components C1r, C1s, factor H, and C2m, as well as the nonimmunologic molecules factor XIII and β2-glycoprotein. A typical Sushi domain has approximately 60 amino acid residues and contains four cysteines (Ranganathan, Pac. Symp Biocomput. 2000:155-67). The first cysteine can form a disulfide bond with the third cysteine, and the second cysteine can form a disulfide bridge with the fourth cysteine. In some embodiments in which one member of the pair of affinity domains is a soluble IL-15, the soluble IL-15 has a D8N or D8A amino acid substitution. In some embodiments in which one member of the pair of affinity domains is an alpha chain of human IL-15 receptor (IL-15Rα), the human IL-15Ra is a mature full-length IL-15Rα. In some embodiments, the pair of affinity domains is barnase and barnstar. In some embodiments, the pair of affinity domains is a PKA and an AKAP. In some embodiments, the pair of affinity domains is an adapter/docking tag module based on mutated RNase I fragments (Rossi, Proc Natl Acad Sci USA. 103:6841-6846, 2006; Sharkey et al., Cancer Res. 68:5282-5290, 2008; Rossi et al., Trends Pharmacol Sci. 33:474-481, 2012) or SNARE modules based on interactions of the proteins syntaxin, synaptotagmin, synaptobrevin, and SNAP25 (Deyev et al., Nat Biotechnol. 1486-1492, 2003).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains and the second domain of the pair of affinity domains bind to each other with a dissociation equilibrium constant (K_{D}) of less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the first domain of the pair of affinity domains and the second domain of the pair of affinity domains bind to each other with a K_{D} of about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰ M, about 1×10⁻⁹ M to about 1×10⁻¹¹ M, about 1×10⁻¹⁰ M to about 1×10⁻¹² M, about 1×10⁻¹¹ M to about 1×10⁻¹³ M, about 1×10⁻⁴ M to about 1×10⁻⁵ M, about 1×10⁻⁵ M to about 1×10⁻⁶ M, about 1×10⁻⁶ M to about 1×10⁻⁷ M, about 1×10⁻⁷ M to about 1×10⁻⁸ M, about 1×10⁻⁸ M to about 1×10⁻⁹ M, about 1×10⁻⁹ M to about 1×10⁻¹⁰ M, about 1×10⁻¹⁰ M to about 1×10⁻¹¹ M, about 1×10⁻¹¹ M to about 1×10⁻¹² M, or about 1×10⁻¹² M to about 1×10⁻¹³ M (inclusive). Any of a variety of different methods known in the art can be used to determine the K_{D} value of the binding of the first domain of the pair of affinity domains and the second domain of the pair of affinity domains (e.g., an electrophoretic mobility shift assay, a filter binding assay, surface plasmon resonance, and a biomolecular binding kinetics assay, etc.).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains, the second domain of the pair of affinity domains, or both is about 10 to 100 amino acids in length. For example, a first domain of a pair of affinity domains, a second domain of a pair of affinity domains, or both can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a first domain of a pair of affinity domains, a second domain of a pair of affinity domains, or both is about 10, 15, 20, 25, 30, 35, 40, 45, 50, 55, 60, 65, 70, 75, 80, 85, 90, 95, or 100 amino acids in length.

In some embodiments, any of the first and/or second domains of a pair of affinity domains disclosed herein can include one or more additional amino acids (e.g., 1, 2, 3, 5, 6, 7, 8, 9, 10, or more amino acids) at its N-terminus and/or C-terminus, so long as the function of the first and/or second domains of a pair of affinity domains remains intact. For example, a sushi domain from an alpha chain of human IL-15 receptor (IL-15Rα) can include one or more additional amino acids at the N-terminus and/or the C-terminus, while still retaining the ability to bind to a soluble IL-15. Additionally or alternatively, a soluble IL-15 can include one or more additional amino acids at the N-terminus and/or the C-terminus, while still retaining the ability to bind to a sushi domain from an alpha chain of human IL-15 receptor (IL-15Rα).

### Exemplary Pairs of Affinity Domains

In some embodiments, a first affinity domain and a second affinity domain of a pair of affinity domains can be a sushi domain from an alpha chain IL-15 receptor alpha and a soluble IL-15.

A non-limiting example of a sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to:

(SEQ ID NO: 11)
ITCPPPMSVEHADIWVKSYSLYSRERYICNSGFKRKAGTSSLTECVLNK
ATNVAHWTTPSLKCIR.

In some embodiments, a sushi domain from an alpha chain of IL-15Ra can be encoded by a nucleic acid including:

(SEQ ID NO: 12)
ATCACGTGCCCTCCCCCCATGTCCGTGGAACACGCAGACATCTGGGTCA
AGAGCTACAGCTTGTACTCCAGGGAGCGGTACATTTGTAACTCTGGTTT
CAAGCGTAAAGCCGGCACGTCCAGCCTGACGGAGTGCGTGTTGAACAAG
GCCACGAATGTCGCCCACTGGACAACCCCCAGTCTCAAATGCATTAGA
G,
or
(SEQ ID NO: 75)
ATTACATGCCCCCCTCCCATGAGCGTGGAGCACGCCGACATCTGGGTGA
AGAGCTATAGCCTCTACAGCCGGGAGAGGTATATCTGTAACAGCGGCTT
CAAGAGGAAGGCCGGCACCAGCAGCCTCACCGAGTGCGTGCTGAATAAG
GCTACCAACGTGGCTCACTGGACAACACCCTCTTTAAAGTGCATCCGG.

In some embodiments, a sushi domain from an alpha chain of IL-15 receptor (IL-15Rα) can include a sequence that is at least 70% identical, at least 75% identical, at least 80% identical, at least 85% identical, at least 90% identical, at least 95% identical, at least 99% identical, or 100% identical to:

(SEQ ID NO: 115)
ITCPPPMSVEHADIWVKSYSLYSRERYICNSGFKRKAGTSSLTECVLNK
ATNVAHWTTPSLKCIRDPALVHQR.

In some embodiments, a sushi domain from an alpha chain of IL-15Ra can be encoded by a nucleic acid including:

(SEQ ID NO: 116)
ATCACGTGCCCTCCCCCCATGTCCGTGGAACACGCAGACATCTGGGTCA
AGAGCTACAGCTTGTACTCCAGGGAGCGGTACATTTGTAACTCTGGTTT
CAAGCGTAAAGCCGGCACGTCCAGCCTGACGGAGTGCGTGTTGAACAAG
GCCACGAATGTCGCCCACTGGACAACCCCCAGTCTCAAATGCATTAGAG
ACCCTGCCCTGGTTCACCAAAGG.

In some embodiments, a soluble IL-15 can include a sequence that is at least 70% identical, at least 75% identical, at least 80% identical, at least 85% identical, at least 90% identical, at least 95% identical, at least 99% identical, or 100% identical to:

(SEQ ID NO: 13)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS.

In some embodiments, a soluble IL-15 can be encoded by a nucleic acid including the sequence of:

(SEQ ID NO: 14)
AACTGGGTGAATGTAATAAGTGATTTGAAAAAAATTGAAGATCTTATTC
AATCTATGCATATTGATGCTACTTTATATACGGAAAGTGATGTTCACCC
CAGTTGCAAAGTAACAGCAATGAAGTGCTTTCTCTTGGAGTTACAAGTT
ATTTCACTTGAGTCCGGAGATGCAAGTATTCATGATACAGTAGAAAATC
TGATCATCCTAGCAAACAACAGTTTGTCTTCTAATGGGAATGTAACAGA
ATCTGGATGCAAAGAATGTGAGGAACTGGAGGAAAAAAATATTAAAGAA
TTTTTGCAGAGTTTTGTACATATTGTCCAAATGTTCATCAACACTTCT.

### Linker Sequences

In some embodiments of any of the multi-chain chimeric polypeptides described herein can include one, two, three, four, five, six, seven, eight, nine, or ten linker sequence(s) (e.g., the same or different linker sequences, e.g., any of the exemplary linker sequences described herein or known in the art). In some embodiments of any of the multi-chain chimeric polypeptides described herein can include one, two, three, four, five, six, seven, eight, nine, or ten linker sequence(s) (e.g., the same or different linker sequences, e.g., any of the exemplary linker sequences described herein or known in the art).

In some embodiments, the linker sequence can be a flexible linker sequence. Non-limiting examples of linker sequences that can be used are described in Klein et al., Protein Engineering, Design & Selection Vol. 27, No. 10, pp. 325-330, 2014; Priyanka et al., Protein Sci., 2013 Feb.; 22(2):153-167. In some examples, the linker sequence is a synthetic linker sequence.

In some embodiments, a linker sequence can have a total length of 1 amino acid to about 100 amino acids, 1 amino acid to about 90 amino acids, 1 amino acid to about 80 amino acids, 1 amino acid to about 70 amino acids, 1 amino acid to about 60 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 24 amino acids, 1 amino acid to about 22 amino acids, 1 amino acid to about 20 amino acids, 1 amino acid to about 18 amino acids, 1 amino acid to about 16 amino acids, 1 amino acid to about 14 amino acids, 1 amino acid to about 12 amino acids, 1 amino acid to about 10 amino acids, 1 amino acid to about 8 amino acids, 1 amino acid to about 6 amino acids, 1 amino acid to about 4 amino acids, about 2 amino acids to about 100 amino acids, about 2 amino acids to about 90 amino acids, about 2 amino acids to about 80 amino acids, about 2 amino acids to about 70 amino acids, about 2 amino acids to about 60 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 4 amino acids, about 4 amino acids to about 100 amino acids, about 4 amino acids to about 90 amino acids, about 4 amino acids to about 80 amino acids, about 4 amino acids to about 70 amino acids, about 4 amino acids to about 60 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 6 amino acids to about 100 amino acids, about 6 amino acids to about 90 amino acids, about 6 amino acids to about 80 amino acids, about 6 amino acids to about 70 amino acids, about 6 amino acids to about 60 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 14 amino acids, about 6 amino acids to about 12 amino acids, about 6 amino acids to about 10 amino acids, about 6 amino acids to about 8 amino acids, about 8 amino acids to about 100 amino acids, about 8 amino acids to about 90 amino acids, about 8 amino acids to about 80 amino acids, about 8 amino acids to about 70 amino acids, about 8 amino acids to about 60 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 14 amino acids, about 8 amino acids to about 12 amino acids, about 8 amino acids to about 10 amino acids, about 10 amino acids to about 100 amino acids, about 10 amino acids to about 90 amino acids, about 10 amino acids to about 80 amino acids, about 10 amino acids to about 70 amino acids, about 10 amino acids to about 60 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 14 amino acids, about 10 amino acids to about 12 amino acids, about 12 amino acids to about 100 amino acids, about 12 amino acids to about 90 amino acids, about 12 amino acids to about 80 amino acids, about 12 amino acids to about 70 amino acids, about 12 amino acids to about 60 amino acids, about 12 amino acids to about 50 amino acids, about 12 amino acids to about 45 amino acids, about 12 amino acids to about 40 amino acids, about 12 amino acids to about 35 amino acids, about 12 amino acids to about 30 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 14 amino acids, about 14 amino acids to about 100 amino acids, about 14 amino acids to about 90 amino acids, about 14 amino acids to about 80 amino acids, about 14 amino acids to about 70 amino acids, about 14 amino acids to about 60 amino acids, about 14 amino acids to about 50 amino acids, about 14 amino acids to about 45 amino acids, about 14 amino acids to about 40 amino acids, about 14 amino acids to about 35 amino acids, about 14 amino acids to about 30 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 16 amino acids to about 100 amino acids, about 16 amino acids to about 90 amino acids, about 16 amino acids to about 80 amino acids, about 16 amino acids to about 70 amino acids, about 16 amino acids to about 60 amino acids, about 16 amino acids to about 50 amino acids, about 16 amino acids to about 45 amino acids, about 16 amino acids to about 40 amino acids, about 16 amino acids to about 35 amino acids, about 16 amino acids to about 30 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 100 amino acids, about 18 amino acids to about 90 amino acids, about 18 amino acids to about 80 amino acids, about 18 amino acids to about 70 amino acids, about 18 amino acids to about 60 amino acids, about 18 amino acids to about 50 amino acids, about 18 amino acids to about 45 amino acids, about 18 amino acids to about 40 amino acids, about 18 amino acids to about 35 amino acids, about 18 amino acids to about 30 amino acids, about 18 amino acids to about 25 amino acids, about 18 amino acids to about 24 amino acids, about 18 amino acids to about 22 amino acids, about 18 amino acids to about 20 amino acids, about 20 amino acids to about 100 amino acids, about 20 amino acids to about 90 amino acids, about 20 amino acids to about 80 amino acids, about 20 amino acids to about 70 amino acids, about 20 amino acids to about 60 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 20 amino acids to about 24 amino acids, about 20 amino acids to about 22 amino acids, about 22 amino acids to about 100 amino acids, about 22 amino acids to about 90 amino acids, about 22 amino acids to about 80 amino acids, about 22 amino acids to about 70 amino acids, about 22 amino acids to about 60 amino acids, about 22 amino acids to about 50 amino acids, about 22 amino acids to about 45 amino acids, about 22 amino acids to about 40 amino acids, about 22 amino acids to about 35 amino acids, about 22 amino acids to about 30 amino acids, about 22 amino acids to about 25 amino acids, about 22 amino acids to about 24 amino acids, about 25 amino acids to about 100 amino acids, about 25 amino acids to about 90 amino acids, about 25 amino acids to about 80 amino acids, about 25 amino acids to about 70 amino acids, about 25 amino acids to about 60 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 100 amino acids, about 30 amino acids to about 90 amino acids, about 30 amino acids to about 80 amino acids, about 30 amino acids to about 70 amino acids, about 30 amino acids to about 60 amino acids, about 30 amino acids to about 50 amino acids, about 30 amino acids to about 45 amino acids, about 30 amino acids to about 40 amino acids, about 30 amino acids to about 35 amino acids, about 35 amino acids to about 100 amino acids, about 35 amino acids to about 90 amino acids, about 35 amino acids to about 80 amino acids, about 35 amino acids to about 70 amino acids, about 35 amino acids to about 60 amino acids, about 35 amino acids to about 50 amino acids, about 35 amino acids to about 45 amino acids, about 35 amino acids to about 40 amino acids, about 40 amino acids to about 100 amino acids, about 40 amino acids to about 90 amino acids, about 40 amino acids to about 80 amino acids, about 40 amino acids to about 70 amino acids, about 40 amino acids to about 60 amino acids, about 40 amino acids to about 50 amino acids, about 40 amino acids to about 45 amino acids, about 45 amino acids to about 100 amino acids, about 45 amino acids to about 90 amino acids, about 45 amino acids to about 80 amino acids, about 45 amino acids to about 70 amino acids, about 45 amino acids to about 60 amino acids, about 45 amino acids to about 50 amino acids, about 50 amino acids to about 100 amino acids, about 50 amino acids to about 90 amino acids, about 50 amino acids to about 80 amino acids, about 50 amino acids to about 70 amino acids, about 50 amino acids to about 60 amino acids, about 60 amino acids to about 100 amino acids, about 60 amino acids to about 90 amino acids, about 60 amino acids to about 80 amino acids, about 60 amino acids to about 70 amino acids, about 70 amino acids to about 100 amino acids, about 70 amino acids to about 90 amino acids, about 70 amino acids to about 80 amino acids, about 80 amino acids to about 100 amino acids, about 80 amino acids to about 90 amino acids, or about 90 amino acids to about 100 amino acids.

In some embodiments, the linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS), e.g., 1, 2, 3, 4, 5, 6, 7, 8, 9, or 10 or more GS pairs. In some embodiments, the linker has one or more Gly-Gly-Gly-Ser (GGGS) sequences, e.g., 1, 2, 3, 4, 5, 6, 7, 8, 9, or 10 or more GGGS sequences. In some embodiments, the linker has one or more Gly-Gly-Gly-Gly-Ser (GGGGS) sequences, e.g., 1, 2, 3, 4, 5, 6, 7, 8, 9, or 10 or more GGGGS sequences. In some embodiments, the linker has one or more Gly-Gly-Ser-Gly (GGSG) sequences, e.g., 1, 2, 3, 4, 5, 6, 7, 8, 9, or 10 or more GGSG sequences.

In some embodiments, the linker sequence can comprise or consist of GGGGSGGGGSGGGGS (SEQ ID NO: 76). In some embodiments, the linker sequence can be encoded by a nucleic acid comprising or consisting of:

(SEQ ID NO: 77)
GGCGGTGGAGGATCCGGAGGAGGTGGCTCCGGCGGCGGAGGATCT.

In some embodiments, the linker sequence can comprise or consist of: GGGSGGGS (SEQ ID NO: 78). In some embodiments, the linker sequence comprises or consists of: GGGS (SEQ ID NO: 79).

### Signal Sequences

In some embodiments, a multi-chain chimeric polypeptide includes a first chimeric polypeptide that includes a signal sequence at its N-terminal end. In some embodiments, a multi-chain chimeric polypeptide includes a second chimeric polypeptide that includes a signal sequence at its N-terminal end. In some embodiments, both the first chimeric polypeptide of a multi-chain chimeric polypeptide and a second chimeric polypeptide of the multi-chain chimeric polypeptide include a signal sequence. As will be understood by those of ordinary skill in the art, a signal sequence is an amino acid sequence that is present at the N-terminus of a number of endogenously produced proteins that directs the protein to the secretory pathway (e.g., the protein is directed to reside in certain intracellular organelles, to reside in the cell membrane, or to be secreted from the cell). Signal sequences are heterogeneous and differ greatly in their primary amino acid sequences. However, signal sequences are typically 16 to 30 amino acids in length and include a hydrophilic, usually positively charged N-terminal region, a central hydrophobic domain, and a C-terminal region that contains the cleavage site for signal peptidase.

In some embodiments, a first chimeric polypeptide of a multi-chain chimeric polypeptide, a second chimeric polypeptide of the multi-chain chimeric polypeptide, or both includes a signal sequence having an amino acid sequence:

(SEQ ID NO: 80)
MKWVTFISLLFLESSAYS.

In some embodiments, a first chimeric polypeptide of a multi-chain chimeric polypeptide, a second chimeric polypeptide of the multi-chain chimeric polypeptide, or both includes a signal sequence encoded by the nucleic acid sequence:

(SEQ ID NO: 81)
ATGAAATGGGTGACCTTTATTTCTTTACTGTTCCTCTTTAGCAGCGCCT
ACTCC,
or
(SEQ ID NO: 82)
ATGAAGTGGGTCACATTTATCTCTTTACTGTTCCTCTTCTCCAGCGCCT
ACAGC.

In some embodiments, a first chimeric polypeptide of a multi-chain chimeric polypeptide, a second chimeric polypeptide of the multi-chain chimeric polypeptide, or both includes a signal sequence having an amino acid sequence:

(SEQ ID NO: 83)
MKCLLYLAFLFLGVNC.

In some embodiments, a first chimeric polypeptide of a multi-chain chimeric polypeptide, a second chimeric polypeptide of the multi-chain chimeric polypeptide, or both includes a signal sequence having an amino acid sequence:

(SEQ ID NO: 84)
MGQIVTMFEALPHIIDEVINIVIIVLIIITSIKAVYNFATCGILALVSF
LFLAGRSCG.

In some embodiments, a first chimeric polypeptide of a multi-chain chimeric polypeptide, a second chimeric polypeptide of the multi-chain chimeric polypeptide, or both includes a signal sequence having an amino acid sequence:

(SEQ ID NO: 85)
MPNHQSGSPTGSSDLLLSGKKQRPHLALRRKRRREMRKINRKVRRMNLA
PIKEKTAWQHLQALISEAEEVLKTSQTPQNSLTLFLALLSVLGPPVTG.

In some embodiments, a first chimeric polypeptide of a multi-chain chimeric polypeptide, a second chimeric polypeptide of the multi-chain chimeric polypeptide, or both includes a signal sequence having an amino acid sequence:

(SEQ ID NO: 86)
MDSKGSSQKGSRLLLLLVVSNLLLCQGVVS.

In some embodiments, a first chimeric polypeptide of a multi-chain chimeric polypeptide, a second chimeric polypeptide of the multi-chain chimeric polypeptide, or both includes a signal sequence having an amino acid sequence:

(SEQ ID NO: 137)
MGVKVLFALICIAVAEA.

In some embodiments, a first chimeric polypeptide of a multi-chain chimeric polypeptide, a second chimeric polypeptide of the multi-chain chimeric polypeptide, or both includes a signal sequence having an amino acid sequence:

(SEQ ID NO: 138)
MERTLVCLVVIFLGTVA.

In some embodiments, a first chimeric polypeptide of a multi-chain chimeric polypeptide, a second chimeric polypeptide of the multi-chain chimeric polypeptide, or both includes a signal sequence having an amino acid sequence:

(SEQ ID NO: 139)
MGWSCIILFLVATATGVHS.

Those of ordinary skill in the art will be aware of other appropriate signal sequences for use in a first chimeric polypeptide and/or a second chimeric polypeptide of multi-chain chimeric polypeptides described herein.

In some embodiments, a first chimeric polypeptide of a multi-chain chimeric polypeptide, a second chimeric polypeptide of the multi-chain chimeric polypeptide, or both includes a signal sequence that is about 10 to 100 amino acids in length. For example, a signal sequence can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a signal sequence is about 10, 15, 20, 25, 30, 35, 40, 45, 50, 55, 60, 65, 70, 75, 80, 85, 90, 95, or 100 amino acids in length.

In some embodiments, any of the signal sequences disclosed herein can include one or more additional amino acids (e.g., 1, 2, 3, 5, 6, 7, 8, 9, 10, or more amino acids) at its N-terminus and/or C-terminus, so long as the function of the signal sequence remains intact. For example, a signal sequence having the amino acid sequence MKCLLYLAFLFLGVNC (SEQ ID NO: 83) can include one or more additional amino acids at the N-terminus or C-terminus, while still retaining the ability to direct a first chimeric polypeptide of a multi-chain chimeric polypeptide, a second chimeric polypeptide of the multi-chain chimeric polypeptide, or both to the secretory pathway.

In some embodiments, a first chimeric polypeptide of a multi-chain chimeric polypeptide, a second chimeric polypeptide of the multi-chain chimeric polypeptide, or both includes a signal sequence that directs the multi-chain chimeric polypeptide into the extracellular space. Such embodiments are useful in producing multi-chain chimeric polypeptides that are relatively easy to be isolated and/or purified.

### Peptide Tags

In some embodiments, a multi-chain chimeric polypeptide includes a first chimeric polypeptide that includes a peptide tag (e.g., at the N-terminal end or the C-terminal end of the first chimeric polypeptide). In some embodiments, a multi-chain chimeric polypeptide includes a second chimeric polypeptide that includes a peptide tag (e.g., at the N-terminal end or the C-terminal end of the second chimeric polypeptide). In some embodiments, both the first chimeric polypeptide of a multi-chain chimeric polypeptide and a second chimeric polypeptide of the multi-chain chimeric polypeptide include a peptide tag. In some embodiments, a first chimeric polypeptide of a multi-chain chimeric polypeptide, a second chimeric polypeptide of the multi-chain chimeric polypeptide, or both include two or more peptide tags.

Exemplary peptide tags that can be included in a first chimeric polypeptide of a multi-chain chimeric polypeptide, a second chimeric polypeptide of the multi-chain chimeric polypeptide, or both include, without limitation, AviTag (GLNDIFEAQKIEWHE; SEQ ID NO: 87), a calmodulin-tag (KRRWKKNFIAVSAANRFKKISSSGAL; SEQ ID NO: 88), a polyglutamate tag (EEEEEE; SEQ ID NO: 89), an E-tag (GAPVPYPDPLEPR; SEQ ID NO: 90), a FLAG-tag (DYKDDDDK; SEQ ID NO: 91), an HA-tag, a peptide from hemagglutinin (YPYDVPDYA; SEQ ID NO: 92), a his-tag (HHHHH (SEQ ID NO: 93); HHHHHH (SEQ ID NO: 94); HHHHHHH (SEQ ID NO: 95); HHHHHHHH (SEQ ID NO: 96); HHHHHHHHH (SEQ ID NO: 97); or HHHHHHHHHH (SEQ ID NO: 98)), a myc-tag (EQKLISEEDL; SEQ ID NO: 99), NE-tag (TKENPRSNQEESYDDNES; SEQ ID NO: 100), S-tag, (KETAAAKFERQHMDS; SEQ ID NO: 101), SBP-tag (MDEKTTGWRGGHVVEGLAGELEQLRARLEHHPQGQREP; SEQ ID NO: 102), Softag 1 (SLAELLNAGLGGS; SEQ ID NO: 103), Softag 3 (TQDPSRVG; SEQ ID NO: 104), Spot-tag (PDRVRAVSHWSS; SEQ ID NO: 105), Strep-tag (WSHPQFEK; SEQ ID NO: 106), TC tag (CCPGCC; SEQ ID NO: 107), Ty tag (EVHTNQDPLD; SEQ ID NO: 108), V5 tag (GKPIPNPLLGLDST; SEQ ID NO: 109), VSV-tag (YTDIEMNRLGK; SEQ ID NO: 110), and Xpress tag (DLYDDDDK; SEQ ID NO: 111). In some embodiments, the peptide tag is a tissue factor protein. In some embodiments, the peptide tag is a TRβC domain.

Peptide tags that can be included in a first chimeric polypeptide of a multi-chain chimeric polypeptide, a second chimeric polypeptide of the multi-chain chimeric polypeptide, or both can be used in any of a variety of applications related to the multi-chain chimeric polypeptide. For example, a peptide tag can be used in the purification of a multi-chain chimeric polypeptide. As one non-limiting example, a first chimeric polypeptide of a multi-chain chimeric polypeptide (e.g., a recombinantly expressed first chimeric polypeptide), a second chimeric polypeptide of the multi-chain chimeric polypeptide (e.g., a recombinantly expressed second chimeric polypeptide), or both can include a myc tag; the multi-chain chimeric polypeptide that includes the myc-tagged first chimeric polypeptide, the myc-tagged second chimeric polypeptide, or both can be purified using an antibody that recognizes the myc tag(s). One non-limiting example of an antibody that recognizes a myc tag is 9E10, available from the non-commercial Developmental Studies Hybridoma Bank. As another non-limiting example, a first chimeric polypeptide of a multi-chain chimeric polypeptide (e.g., a recombinantly expressed first chimeric polypeptide), a second chimeric polypeptide of the multi-chain chimeric polypeptide (e.g., a recombinantly expressed second chimeric polypeptide), or both can include a histidine tag; the multi-chain chimeric polypeptide that includes the histidine-tagged first chimeric polypeptide, the histidine-tagged second chimeric polypeptide, or both can be purified using a nickel or cobalt chelate.

Those of ordinary skill in the art will be aware of other suitable tags and agent that bind those tags for use in purifying multi-chain chimeric polypeptide. In some embodiments, a peptide tag is removed from the first chimeric polypeptide and/or the second chimeric polypeptide of the multi-chain chimeric polypeptide after purification. In some embodiments, a peptide tag is not removed from the first chimeric polypeptide and/or the second chimeric polypeptide of the multi-chain chimeric polypeptide after purification.

In some embodiments, one or both of the first target-binding domain and the second target-binding domain can be used in the purification of a single-chain chimeric polypeptide. In some embodiments, wherein one or both of the first target-binding domain and the second target-binding domain is a tissue factor protein, the first target-binding domain and/or the second target-binding domain is used in the purification of a single-chain chimeric polypeptide. In some embodiments, the TRβC domain is used in the purification of a single-chain chimeric polypeptide. For example, a single-chain chimeric polypeptide can be purified using an antibody that recognizes the TRβC domain.

Peptide tags that can be included in a first chimeric polypeptide of a multi-chain chimeric polypeptide, a second chimeric polypeptide of the multi-chain chimeric polypeptide, or both can be used, for example, in immunoprecipitation of the multi-chain chimeric polypeptide, imaging of the multi-chain chimeric polypeptide (e.g., via Western blotting, ELISA, flow cytometry, and/or immunocytochemistry), and/or solubilization of the multi-chain chimeric polypeptide.

In some embodiments, a first chimeric polypeptide of a multi-chain chimeric polypeptide, a second chimeric polypeptide of the multi-chain chimeric polypeptide, or both includes a peptide tag that is about 10 to 200 amino acids in length. For example, a peptide tag can be about 10 to 200 amino acids in length, about 15 to 200 amino acids in length, about 20 to 200 amino acids in length, about 25 to 200 amino acids in length, about 30 to 200 amino acids in length, about 35 to 200 amino acids in length, about 40 to 200 amino acids in length, about 45 to 200 amino acids in length, about 50 to 200 amino acids in length, about 55 to 200 amino acids in length, about 60 to 200 amino acids in length, about 65 to 200 amino acids in length, about 70 to 200 amino acids in length, about 75 to 200 amino acids in length, about 80 to 200 amino acids in length, about 85 to 200 amino acids in length, about 90 to 200 amino acids in length, about 95 to 200 amino acids in length, about 100 to 200 amino acids in length, about 110 to 200 amino acids in length, about 120 to 200 amino acids in length, about 130 to 200 amino acids in length, about 140 to 200 amino acids in length, about 150 to 200 amino acids in length, about 160 to 200 amino acids in length, about 170 to 200 amino acids in length, about 180 to 200 amino acids in length, about 190 to 200 amino acids in length, about 10 to 190 amino acids in length, about 15 to 190 amino acids in length, about 20 to 190 amino acids in length, about 25 to 190 amino acids in length, about 30 to 190 amino acids in length, about 35 to 190 amino acids in length, about 40 to 190 amino acids in length, about 45 to 190 amino acids in length, about 50 to 190 amino acids in length, about 55 to 190 amino acids in length, about 60 to 190 amino acids in length, about 65 to 190 amino acids in length, about 70 to 190 amino acids in length, about 75 to 190 amino acids in length, about 80 to 190 amino acids in length, about 85 to 190 amino acids in length, about 90 to 190 amino acids in length, about 95 to 190 amino acids in length, about 100 to 190 amino acids in length, about 110 to 190 amino acids in length, about 120 to 190 amino acids in length, about 130 to 190 amino acids in length, about 140 to 190 amino acids in length, about 150 to 190 amino acids in length, about 160 to 190 amino acids in length, about 170 to 190 amino acids in length, about 180 to 190 amino acids in length, about 10 to 180 amino acids in length, about 15 to 180 amino acids in length, about 20 to 180 amino acids in length, about 25 to 180 amino acids in length, about 30 to 180 amino acids in length, about 35 to 180 amino acids in length, about 40 to 180 amino acids in length, about 45 to 180 amino acids in length, about 50 to 180 amino acids in length, about 55 to 180 amino acids in length, about 60 to 180 amino acids in length, about 65 to 180 amino acids in length, about 70 to 180 amino acids in length, about 75 to 180 amino acids in length, about 80 to 180 amino acids in length, about 85 to 180 amino acids in length, about 90 to 180 amino acids in length, about 95 to 180 amino acids in length, about 100 to 180 amino acids in length, about 110 to 180 amino acids in length, about 120 to 180 amino acids in length, about 130 to 180 amino acids in length, about 140 to 180 amino acids in length, about 150 to 180 amino acids in length, about 160 to 180 amino acids in length, about 170 to 180 amino acids in length, about 10 to 170 amino acids in length, about 15 to 170 amino acids in length, about 20 to 170 amino acids in length, about 25 to 170 amino acids in length, about 30 to 170 amino acids in length, about 35 to 170 amino acids in length, about 40 to 170 amino acids in length, about 45 to 170 amino acids in length, about 50 to 170 amino acids in length, about 55 to 170 amino acids in length, about 60 to 170 amino acids in length, about 65 to 170 amino acids in length, about 70 to 170 amino acids in length, about 75 to 170 amino acids in length, about 80 to 170 amino acids in length, about 85 to 170 amino acids in length, about 90 to 170 amino acids in length, about 95 to 170 amino acids in length, about 100 to 170 amino acids in length, about 110 to 170 amino acids in length, about 120 to 170 amino acids in length, about 130 to 170 amino acids in length, about 140 to 170 amino acids in length, about 150 to 170 amino acids in length, about 160 to 170 amino acids in length, about 10 to 160 amino acids in length, about 15 to 160 amino acids in length, about 20 to 160 amino acids in length, about 25 to 160 amino acids in length, about 30 to 160 amino acids in length, about 35 to 160 amino acids in length, about 40 to 160 amino acids in length, about 45 to 160 amino acids in length, about 50 to 160 amino acids in length, about 55 to 160 amino acids in length, about 60 to 160 amino acids in length, about 65 to 160 amino acids in length, about 70 to 160 amino acids in length, about 75 to 160 amino acids in length, about 80 to 160 amino acids in length, about 85 to 160 amino acids in length, about 90 to 160 amino acids in length, about 95 to 160 amino acids in length, about 100 to 160 amino acids in length, about 110 to 160 amino acids in length, about 120 to 160 amino acids in length, about 130 to 160 amino acids in length, about 140 to 160 amino acids in length, about 150 to 160 amino acids in length, about 10 to 150 amino acids in length, about 15 to 150 amino acids in length, about 20 to 150 amino acids in length, about 25 to 150 amino acids in length, about 30 to 150 amino acids in length, about 35 to 150 amino acids in length, about 40 to 150 amino acids in length, about 45 to 150 amino acids in length, about 50 to 150 amino acids in length, about 55 to 150 amino acids in length, about 60 to 150 amino acids in length, about 65 to 150 amino acids in length, about 70 to 150 amino acids in length, about 75 to 150 amino acids in length, about 80 to 150 amino acids in length, about 85 to 150 amino acids in length, about 90 to 150 amino acids in length, about 95 to 150 amino acids in length, about 100 to 150 amino acids in length, about 110 to 150 amino acids in length, about 120 to 150 amino acids in length, about 130 to 150 amino acids in length, about 140 to 150 amino acids in length, about 10 to 140 amino acids in length, about 15 to 140 amino acids in length, about 20 to 140 amino acids in length, about 25 to 140 amino acids in length, about 30 to 140 amino acids in length, about 35 to 140 amino acids in length, about 40 to 140 amino acids in length, about 45 to 140 amino acids in length, about 50 to 140 amino acids in length, about 55 to 140 amino acids in length, about 60 to 140 amino acids in length, about 65 to 140 amino acids in length, about 70 to 140 amino acids in length, about 75 to 140 amino acids in length, about 80 to 140 amino acids in length, about 85 to 140 amino acids in length, about 90 to 140 amino acids in length, about 95 to 140 amino acids in length, about 100 to 140 amino acids in length, about 110 to 140 amino acids in length, about 120 to 140 amino acids in length, about 130 to 140 amino acids in length, about 10 to 130 amino acids in length, about 15 to 130 amino acids in length, about 20 to 130 amino acids in length, about 25 to 130 amino acids in length, about 30 to 130 amino acids in length, about 35 to 130 amino acids in length, about 40 to 130 amino acids in length, about 45 to 130 amino acids in length, about 50 to 130 amino acids in length, about 55 to 130 amino acids in length, about 60 to 130 amino acids in length, about 65 to 130 amino acids in length, about 70 to 130 amino acids in length, about 75 to 130 amino acids in length, about 80 to 130 amino acids in length, about 85 to 130 amino acids in length, about 90 to 130 amino acids in length, about 95 to 130 amino acids in length, about 100 to 130 amino acids in length, about 110 to 130 amino acids in length, about 120 to 130 amino acids in length, about 10 to 120 amino acids in length, about 15 to 120 amino acids in length, about 20 to 120 amino acids in length, about 25 to 120 amino acids in length, about 30 to 120 amino acids in length, about 35 to 120 amino acids in length, about 40 to 120 amino acids in length, about 45 to 120 amino acids in length, about 50 to 120 amino acids in length, about 55 to 120 amino acids in length, about 60 to 120 amino acids in length, about 65 to 120 amino acids in length, about 70 to 120 amino acids in length, about 75 to 120 amino acids in length, about 80 to 120 amino acids in length, about 85 to 120 amino acids in length, about 90 to 120 amino acids in length, about 95 to 120 amino acids in length, about 100 to 120 amino acids in length, about 110 to 120 amino acids in length, bout 10 to 110 amino acids in length, about 15 to 110 amino acids in length, about 20 to 110 amino acids in length, about 25 to 110 amino acids in length, about 30 to 110 amino acids in length, about 35 to 110 amino acids in length, about 40 to 110 amino acids in length, about 45 to 110 amino acids in length, about 50 to 110 amino acids in length, about 55 to 110 amino acids in length, about 60 to 110 amino acids in length, about 65 to 110 amino acids in length, about 70 to 110 amino acids in length, about 75 to 110 amino acids in length, about 80 to 110 amino acids in length, about 85 to 110 amino acids in length, about 90 to 110 amino acids in length, about 95 to 110 amino acids in length, about 100 to 110 amino acids in length, about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a peptide tag is about 10, 15, 20, 25, 30, 35, 40, 45, 50, 55, 60, 65, 70, 75, 80, 85, 90, 95, or 100 amino acids in length.

Peptide tags included in a first chimeric polypeptide of a multi-chain chimeric polypeptide, a second chimeric polypeptide of the multi-chain chimeric polypeptide, or both can be of any suitable length. For example, peptide tags can be 5, 6, 7, 8, 9, 10, 11, 12, 13, 14, 15, 16, 17, 18, 19, 20, or more amino acids in length. In embodiments in which a multi-chain chimeric polypeptide includes two or more peptide tags, the two or more peptide tags can be of the same or different lengths. In some embodiments, any of the peptide tags disclosed herein may include one or more additional amino acids (e.g., 1, 2, 3, 5, 6, 7, 8, 9, 10, or more amino acids) at the N-terminus and/or C-terminus, so long as the function of the peptide tag remains intact. For example, a myc tag having the amino acid sequence EQKLISEEDL (SEQ ID NO: 112) can include one or more additional amino acids (e.g., at the N-terminus and/or the C-terminus of the peptide tag), while still retaining the ability to be bound by an antibody (e.g., 9E10).

### Exemplary Multi-Chain Chimeric Polypeptides—Type A

In some embodiments of any of the multi-chain chimeric polypeptides described herein, the first target-binding domain and the second targeting-binding domain each independently bind specifically to a receptor of IL-7 or a ligand of TGF-βRII. In some embodiments of these multi-chain chimeric polypeptides, the first target-binding domain and the second target-binding domain both bind specifically to a receptor of IL-7 or a ligand of TGF-βRII.

In some embodiments of these multi-chain chimeric polypeptides, the first target-binding domain binds specifically to a receptor for IL-7, and the second target-binding domain binds specifically to a ligand of TGF-βRII. In some embodiments of these multi-chain chimeric polypeptides, the first target-binding domain binds specifically to a ligand of TGF-βRII, and the second target-binding domain bind specifically to a receptor for IL-7.

In some examples of these multi-chain chimeric polypeptides, the first target-binding domain and the soluble TRβC domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble TRβC domain in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments, the second chimeric polypeptide further comprises one or more additional target-binding domains at the N-terminal end or the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

In some embodiments of these multi-chain chimeric polypeptides, the additional target-binding domain and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second target-binding domain and the additional target-binding domain in the second chimeric polypeptide.

In some embodiments of these multi-chain chimeric polypeptides, the soluble T cell receptor β-chain constant region (TRβC) domain can be any of the exemplary soluble T cell receptor β-chain constant region (TRβC) domains described herein.

In some embodiments of these multi-chain chimeric polypeptides, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these multi-chain chimeric polypeptides, the pair of affinity domains can be any of the exemplary pairs of affinity domains described herein. In some embodiments, the pair of affinity domains is a sushi domain from an alpha chain of human IL-15 receptor (IL-15Rα) and a soluble IL-15. In some embodiments, the second domain of the pair of affinity domains is a sushi domain from an alpha chain of human IL-15 receptor (IL-15Rα) and the first domain of the pair of affinity domains a soluble IL-15. In some embodiments, the first domain of the pair of affinity domains is a sushi domain from an alpha chain of human IL-15 receptor (IL-15Rα) and the second domain of the pair of affinity domains a soluble IL-15.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 11.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 115.

In some embodiments, a soluble IL-15 can include a sequence that is at least 70% identical, at least 75% identical, at least 80% identical, at least 85% identical, at least 90% identical, at least 95% identical, at least 99% identical, or 100% identical to SEQ ID NO: 13.

In some embodiments of these multi-chain chimeric polypeptides, one or both of the first target-binding domain and the second target-binding domain is a soluble IL-7 or a soluble TGFβRII receptor. In some embodiments of these multi-chain chimeric polypeptides, the first target-binding domain and the second target-binding domain are each independently a soluble IL-7 or a soluble TGFβRII receptor.

In some embodiments of these multi-chain chimeric polypeptides, the first target-binding domain is a soluble IL-7, and the second target-binding domain is a soluble TGFβRII receptor. In some embodiments of these multi-chain chimeric polypeptides, the first target-binding domain is a soluble IL-7, and the second target-binding domain is a dimeric TGF-βRII receptor in a single-chain format. In some embodiments of these multi-chain chimeric polypeptides, the first target-binding domain is a soluble TGFβRII receptor, and the second target-binding domain is a soluble IL-7. In some embodiments of these multi-chain chimeric polypeptides, the first target-binding domain is a soluble TGFβRII receptor in a single-chain format, and the second target-binding domain is a soluble IL-7.

A non-limiting example of an IL-7 protein that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

A non-limiting example of a soluble TGFβRII receptor that binds specifically to a ligand of TGF-βRII can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7, and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 49, or SEQ ID NO: 50, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to: SEQ ID NO: 8, SEQ ID NO: 49, or SEQ ID NO: 50.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 51.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10 or SEQ ID NO: 52.

In some embodiments, the second chimeric polypeptide further comprises one or more additional target-binding domains at the N-terminal end or the C-terminal end of the second chimeric polypeptide. In some embodiments, the second chimeric polypeptide further comprises one or more additional target-binding domains at the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

In some embodiments, the additional target-binding domain includes or is an anti-tissue factor antigen-binding domain or an anti-PD-1 antigen-binding domain.

In some embodiments, the additional target-binding domain includes or is an anti-tissue factor antigen-binding domain. In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 53.

In some embodiments, the anti-tissue factor antigen-binding domain can further include a linker sequence (e.g., any of the exemplary linker sequences described herein or known in the art) between the first sequence and the second sequence.

In some embodiments of any of the multi-chain chimeric polypeptides described herein, the additional target-binding domain includes or is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 15.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 16.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 129, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 130.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 131.

In some embodiments, the additional target-binding domain includes or is an anti-PD-1 antigen-binding domain. In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 64.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 21.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 22.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 134.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 135.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 136.

In some embodiments, the anti-PD-1 antigen-binding domain is an anti-PD-1 single chain diabody. In some embodiments, the anti-PD-1 antigen-binding domain is a human anti-PD-1 diabody. In some embodiments, the anti-PD-1 antigen-binding domain is a mouse anti-PD-1 diabody.

### IL-7/TRβC/IL-15: TGFβRII/IL-1SRαSu/Anti-PD-1(Diabody)

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-PD-1 diabody (e.g., a sequence that is at least 80%, at least 90%, at least 95%, or 100% identical to SEQ ID NO: 21).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 141)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIRQ
VQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARR
DYRFDMGFDYWGQGTTVTVSSGGGGSEIVLTQSPATLSLSPGERATLSC
RASKGVSTSGYSYLHWYQQKPGQAPRLLIYLASYLESGVPARFSGSGSG
TDFTLTISSLEPEDFAVYYCQHSRDLPLTFGGGTKVEIKGGGGSGGGGS
GGGGSQVQLVQSGVEVKKPGASVKVSCKASGYTFTNYYMYWVRQAPGQG
LEWMGGINPSNGGTNFNEKFKNRVTLTTDSSTTTAYMELKSLQFDDTAV
YYCARRDYRFDMGFDYWGQGTTVTVSSGGGGSEIVLTQSPATLSLSPGE
RATLSCRASKGVSTSGYSYLHWYQQKPGQAPRLLIYLASYLESGVPARF
SGSGSGTDFTLTISSLEPEDFAVYYCQHSRDLPLTFGGGTKVEIK.

In some embodiments, the second chimeric polypeptide further includes a signal sequence at its N-terminal end. In some embodiments the signal peptide sequence is MKWVTFISLLFLFSSAYS (SEQ ID NO: 80).

### IL-7/TRβC/IL-15: TGFβRII/IL-15RαSu

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 142)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIR.

In some embodiments, the second chimeric polypeptide further includes a signal sequence at its N-terminal end. In some embodiments the signal peptide sequence is MKWVTFISLLFLFSSAYS (SEQ ID NO: 80).

### IL-7/TRβC/IL-15: TGFβRII/IL-1SRαSu/Anti-Mouse PD-1(Diabody)

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a mouse anti-PD-1 diabody (e.g., a sequence that is at least 80%, at least 90%, at least 95%, or 100% identical to SEQ ID NO: 134).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 143)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIRQ
VQLQQSGAELVKPGSSVKISCKASGYTFTSHFIHWIKQQPGNGLEWIGG
IYPGDGDTEYNQQFNGKATLTADKSSSTAYMRLSSLTSEDSAVYFCATR
VPSYWFFDFWGPGTMVTVSSGGGGSRTDVALTQTPVAQPVTLGDQASIS
CRSSQSLVHSNGRTYLEWYLQKPGQSPQLLIYKVSNRFSGVPDRFIGSG
SGSDFTLTISRVEPEDLGVYYCFQATHDPNTFGAGTKLELKGGGGSGGG
GSGGGGSQVQLQQSGAELVKPGSSVKISCKASGYTFTSHFIHWIKQQPG
NGLEWIGGIYPGDGDTEYNQQFNGKATLTADKSSSTAYMRLSSLTSEDS
AVYFCATRVPSYWFFDFWGPGTMVTVSSGGGGSRTDVALTQTPVAQPVT
LGDQASISCRSSQSLVHSNGRTYLEWYLQKPGQSPQLLIYKVSNRFSGV
PDRFIGSGSGSDFTLTISRVEPEDLGVYYCFQATHDPNTFGAGTKLEL
K.

In some embodiments, the second chimeric polypeptide further includes a signal sequence at its N-terminal end. In some embodiments the signal peptide sequence is MGVKVLFALICIAVAEA (SEQ ID NO: 137).

### IL-7/TRβC/IL-15: TGFβRII/IL-15RαSu/Anti-TF

In some embodiments, provided herein is a multi-chain chimeric polypeptide that includes: (a) a first chimeric polypeptide including: (i) a first target-binding domain that includes a soluble human IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human TF antigen-binding domain. In some embodiments, the anti-human TF antigen-binding domain is an anti-human TF diabody (e.g., a sequence that is at least 80%, at least 90%, at least 95%, or 100% identical to SEQ ID NO: 15). In some embodiments, the anti-human TF antigen-binding domain is an anti-human TFscFv (e.g., a sequence that is at least 80%, at least 90%, at least 95%, or 100% identical to SEQ ID NO: 131).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 144)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIRD
IQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFG
QGTKLEIKGGGGSQIQLVQSGGEVKKPGASVRVSCKASGYSFTDYNVYW
VRQSPGKGLEWIGYIDPYNGITIYDQNFKGKATLTVDKSTSTAYMELSS
LRSEDTAVYFCARDVTTALDFWGQGTTVTVSSGGGGSGGGGSGGGGSDI
QMTQSPASLSASVGDRVTITCLASQTIDTWLAWYLQKPGKSPQLLIYAA
TNLADGVPSRFSGSGSGTDFSFTISSLQPEDFATYYCQQVYSSPFTFGQ
GTKLEIKGGGGSQIQLVQSGGEVKKPGASVRVSCKASGYSFTDYNVYWV
RQSPGKGLEWIGYIDPYNGITIYDQNFKGKATLTVDKSTSTAYMELSSL
RSEDTAVYFCARDVTTALDFWGQGTTVTVSS.

In some embodiments, the second chimeric polypeptide further includes a signal sequence at its N-terminal end. In some embodiments the signal peptide sequence is MKWVTFISLLFLFSSAYS (SEQ ID NO: 80).
IL-7/TRβC/IL-15: TGFβRII/IL-15RαSu/Anti-PD-1 scFv

In some embodiments, provided herein is a multi-chain chimeric polypeptide that includes: (a) a first chimeric polypeptide including: (i) a first target-binding domain that includes a soluble human IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-PD-1 scFv (e.g., a sequence that is at least 80%, at least 90%, at least 95%, or 100% identical to SEQ ID NO: 135 or SEQ ID NO: 136).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 145)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIRG
GGGSEIVLTQSPATLSLSPGERATLSCRASKGVSTSGYSYLHWYQQKPG
QAPRLLIYLASYLESGVPARFSGSGSGTDFTLTISSLEPEDFAVYYCQH
SRDLPLTFGGGTKVEIKGGGGSGGGGSGGGGSQVQLVQSGVEVKKPGAS
VKVSCKASGYTFTNYYMYWVRQAPGQGLEWMGGINPSNGGTNFNEKFKN
RVTLTTDSSTTTAYMELKSLQFDDTAVYYCARRDYRFDMGFDYWGQGTT
VTVSS.

In some embodiments, the second chimeric polypeptide further includes a signal sequence at its N-terminal end. In some embodiments the signal peptide sequence is MKWVTFISLLFLFSSAYS (SEQ ID NO: 80).

### Exemplary Multi-Chain Chimeric Polypeptides—Type B

In some embodiments of any of the multi-chain chimeric polypeptides described herein, the first target-binding domain and the second targeting-binding domain each independently bind specifically to an IL-7 receptor or to an IL-21 receptor. In some embodiments of these multi-chain chimeric polypeptides, the first target-binding domain and the second target-binding domain both bind specifically to an IL-7 receptor or to an IL-21 receptor.

In some embodiments of these multi-chain chimeric polypeptides, the first target-binding domain binds specifically to an IL-7 receptor, and the second target-binding domain binds specifically to an IL-21 receptor. In some embodiments of these multi-chain chimeric polypeptides, the first target-binding domain binds specifically to an IL-21 receptor, and the second target-binding domain bind specifically to an IL-7 receptor.

In some examples of these multi-chain chimeric polypeptides, the first target-binding domain and the soluble TRβC domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble TRβC domain in the first chimeric polypeptide.

In some embodiments of these multi-chain chimeric polypeptides, the soluble TRβC domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble TRβC domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble T cell receptor β-chain constant region (TRβC) domain can be any of the exemplary soluble T cell receptor β-chain constant region (TRβC) domains described herein.

In some embodiments of these multi-chain chimeric polypeptides, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these multi-chain chimeric polypeptides, the pair of affinity domains can be any of the exemplary pairs of affinity domains described herein. In some embodiments, the pair of affinity domains is a sushi domain from an alpha chain of human IL-15 receptor (IL-15Rα) and a soluble TL-15. In some embodiments, the second domain of the pair of affinity domains is a sushi domain from an alpha chain of human IL-15 receptor (IL-15Rα) and the first domain of the pair of affinity domains a soluble IL-15. In some embodiments, the first domain of the pair of affinity domains is a sushi domain from an alpha chain of human IL-15 receptor (IL-15Rα) and the second domain of the pair of affinity domains a soluble IL-15.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 11.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 115.

In some embodiments, a soluble IL-15 can include a sequence that is at least 70% identical, at least 75% identical, at least 80% identical, at least 85% identical, at least 90% identical, at least 95% identical, at least 99% identical, or 100% identical to SEQ ID NO: 13.

In some embodiments of these multi-chain chimeric polypeptides, one or both of the first target-binding domain and the second target-binding domain is a soluble IL-7 (e.g., a soluble human IL-7 polypeptide) or a soluble IL-21 (e.g., a soluble IL-21 polypeptide). In some embodiments of these multi-chain chimeric polypeptides, the first target-binding domain and the second target-binding domain are each independently a soluble IL-7 or a soluble IL-21. In some embodiments of these multi-chain chimeric polypeptides, the first target-binding domain is a soluble IL-7 and the second target-binding domain is a soluble IL-21. In some embodiments of these multi-chain chimeric polypeptides, the first target-binding domain is a soluble IL-21 and the second target-binding domain is a soluble human IL-7. In some embodiments of these multi-chain chimeric polypeptides, the first target-binding domain is a soluble IL-7 and the second target-binding domain is a soluble mouse IL-21. In some embodiments of these multi-chain chimeric polypeptides, the first target-binding domain and the second target-binding domain bind specifically to the same epitope. In some embodiments of these multi-chain chimeric polypeptides, the first target-binding domain and the second target-binding domain include the same amino acid sequence.

In some embodiments of these multi-chain chimeric polypeptides, the first target-binding domain is a soluble human IL-7, and the second target-binding domain is a soluble human IL-21. In some embodiments of these multi-chain chimeric polypeptides, the first target-binding domain is a soluble human IL-21, and the second target-binding domain is a soluble human IL-7.

A non-limiting example of an IL-7 that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

A non-limiting example of an IL-21 that binds specifically to an IL-21 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 5.

In some embodiments, an IL-21 that binds specifically to an IL-21 receptor can be encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 6 or 23.

A non-limiting example of an IL-21 that binds specifically to an IL-21 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 124.

In some embodiments, the second chimeric polypeptide further comprises one or more additional target-binding domains at the N-terminal end or the C-terminal end of the second chimeric polypeptide. In some embodiments, the second chimeric polypeptide further comprises one or more additional target-binding domains at the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

In some embodiments, the additional target-binding domain includes or is an anti-tissue factor antigen-binding domain or an anti-PD-1 antigen-binding domain.

In some embodiments, the additional target-binding domain includes or is an anti-tissue factor antigen-binding domain. In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 53.

In some embodiments, the anti-tissue factor antigen-binding domain can further include a linker sequence (e.g., any of the exemplary linker sequences described herein or known in the art) between the first sequence and the second sequence.

In some embodiments of any of the multi-chain chimeric polypeptides described herein, the additional target-binding domain includes or is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 15.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 16.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 129, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 130.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 131.

In some embodiments, the additional target-binding domain includes or is an anti-PD-1 antigen-binding domain. In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 64.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 21.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 22.

In some embodiments, the anti-PD-1 antigen-binding domain is a mouse anti-PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 134.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 135.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 136.

### IL-7/TRβC/IL-15: IL-21/IL-15RαSu

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a human soluble IL-21 (e.g., a sequence that is at least 80%, at least 90%, at least 95%, or 100% identical to SEQ ID NO: 5); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 147)
QGQDRHMIRMRQLIDIVDQLKNYVNDLVPEFLPAPEDVETNCEWSAFSC
FQKAQLKSANTGNNERIINVSIKKLKRKPPSTNAGRRQKHRLTCPSCDS
YEKKPPKEFLERFKSLLQKMIHQHLSSRTHGSEDSITCPPPMSVEHADI
WVKSYSLYSRERYICNSGFKRKAGTSSLTECVLNKATNVAHWTTPSLKC
IR.

In some embodiments, the second chimeric polypeptide further includes a signal sequence at its N-terminal end.

In some embodiments, the first chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 148)
GATTGTGATATTGAAGGTAAAGATGGCAAACAATATGAGAGTGTTCTAA
TGGTCAGCATCGATCAATTATTGGACAGCATGAAAGAAATTGGTAGCAA
TTGCCTGAATAATGAATTTAACTTTTTTAAAAGACATATCTGTGATGCT
AATAAGGAAGGTATGTTTTTATTCCGTGCTGCTCGCAAGTTGAGGCAAT
TTCTTAAAATGAATAGCACTGGTGATTTTGATCTCCACTTATTAAAAGT
TTCAGAAGGCACAACAATACTGTTGAACTGCACTGGCCAGGTTAAAGGA
AGAAAACCAGCTGCCCTGGGTGAAGCCCAACCAACAAAGAGTTTGGAAG
AAAATAAATCTTTAAAGGAACAGAAAAAACTGAATGACTTGTGTTTCCT
AAAGAGACTATTACAAGAGATAAAAACTTGTTGGAATAAAATTTTGATG
GGCACTAAAGAACACGAGGACCTGAACAAGGTGTTCCCACCCGAGGTCG
CTGTGTTTGAGCCATCAGAAGCAGAGATCTCCCACACCCAAAAGGCCAC
ACTGGTGTGCCTGGCCACAGGCTTCTTCCCCGACCACGTGGAGCTGAGC
TGGTGGGTGAATGGGAAGGAGGTGCACAGTGGGGTCAGCACGGACCCGC
AGCCCCTCAAGGAGCAGCCCGCCCTCAATGACTCCAGATACTGCCTGAG
CAGCCGCCTGAGGGTCTCGGCCACCTTCTGGCAGAACCCCCGCAACCAC
TTCCGCTGTCAAGTCCAGTTCTACGGGCTCTCGGAGAATGACGAGTGGA
CCCAGGATAGGGCCAAACCCGTCACCCAGATCGTCAGCGCCGAGGCCTG
GGGTAGAGCAGACAACTGGGTGAATGTAATAAGTGATTTGAAAAAAATT
GAAGATCTTATTCAATCTATGCATATTGATGCTACTTTATATACGGAAA
GTGATGTTCACCCCAGTTGCAAAGTAACAGCAATGAAGTGCTTTCTCTT
GGAGTTACAAGTTATTTCACTTGAGTCCGGAGATGCAAGTATTCATGAT
ACAGTAGAAAATCTGATCATCCTAGCAAACAACAGTTTGTCTTCTAATG
GGAATGTAACAGAATCTGGATGCAAAGAATGTGAGGAACTGGAGGAAAA
AAATATTAAAGAATTTTTGCAGAGTTTTGTACATATTGTCCAAATGTTC
ATCAACACTTCTTGATAACGCGTACGAAG.

In some embodiments, the second chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 149)
CAAGGTCAAGATCGCCACATGATTAGAATGCGTCAACTTATAGATATTG
TTGATCAGCTGAAAAATTATGTGAATGACTTGGTCCCTGAATTTCTGCC
AGCTCCAGAAGATGTAGAGACAAACTGTGAGTGGTCAGCTTTTTCCTGT
TTTCAGAAGGCCCAACTAAAGTCAGCAAATACAGGAAACAATGAAAGGA
TAATCAATGTATCAATTAAAAAGCTGAAGAGGAAACCACCTTCCACAAA
TGCAGGGAGAAGACAGAAACACAGACTAACATGCCCTTCATGTGATTCT
TATGAGAAAAAACCACCCAAAGAATTCCTAGAAAGATTCAAATCACTTC
TCCAAAAGATGATTCATCAGCATCTGTCCTCTAGAACACACGGAAGTGA
AGATTCCATCACGTGCCCTCCCCCCATGTCCGTGGAACACGCAGACATC
TGGGTCAAGAGCTACAGCTTGTACTCCAGGGAGCGGTACATTTGTAACT
CTGGTTTCAAGCGTAAAGCCGGCACGTCCAGCCTGACGGAGTGCGTGTT
GAACAAGGCCACGAATGTCGCCCACTGGACAACCCCCAGTCTCAAATGC
ATTAGATGATAACGCGTACGAAG.

### IL-7/TRβC1/IL-15: IL-21/IL-15RαSu/anti-TF

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a human soluble IL-21 (e.g., a sequence that is at least 80%, at least 90%, at least 95%, or 100% identical to SEQ ID NO: 5); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human TF antigen-binding domain. In some embodiments, the anti-human TF antigen-binding domain is an anti-human TF diabody (e.g., a sequence that is at least 80%, at least 90%, at least 95%, or 100% identical to SEQ ID NO: 15). In some embodiments, the anti-human TF antigen-binding domain is an anti-human TFscFv (e.g., a sequence that is at least 80%, at least 90%, at least 95%, or 100% identical to SEQ ID NO: 131).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS,

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 151)
QGQDRHMIRMRQLIDIVDQLKNYVNDLVPEFLPAPEDVETNCEWSAFSC
FQKAQLKSANTGNNERIINVSIKKLKRKPPSTNAGRRQKHRLTCPSCDS
YEKKPPKEFLERFKSLLQKMIHQHLSSRTHGSEDSITCPPPMSVEHADI
WVKSYSLYSRERYICNSGFKRKAGTSSLTECVLNKATNVAHWTTPSLKC
IRDIQMTQSPASLSASVGDRVTITCLASQTIDTWLAWYLQKPGKSPQLL
IYAATNLADGVPSRFSGSGSGTDFSFTISSLQPEDFATYYCQQVYSSPF
TFGQGTKLEIKGGGGSQIQLVQSGGEVKKPGASVRVSCKASGYSFTDYN
VYWVRQSPGKGLEWIGYIDPYNGITIYDQNFKGKATLTVDKSTSTAYME
LSSLRSEDTAVYFCARDVTTALDFWGQGTTVTVSSGGGGSGGGGSGGGG
SDIQMTQSPASLSASVGDRVTITCLASQTIDTWLAWYLQKPGKSPQLLI
YAATNLADGVPSRFSGSGSGTDFSFTISSLQPEDFATYYCQQVYSSPFT
FGQGTKLEIKGGGGSQIQLVQSGGEVKKPGASVRVSCKASGYSFTDYNV
YWVRQSPGKGLEWIGYIDPYNGITIYDQNFKGKATLTVDKSTSTAYMEL
SSLRSEDTAVYFCARDVTTALDFWGQGTTVTVSS.

In some embodiments, the second chimeric polypeptide further includes a signal sequence at its N-terminal end. In some embodiments the signal peptide sequence is MKWVTFISLLFLFSSAYS (SEQ ID NO: 80).

### IL-7/TRβC/IL-15: Mouse IL-21/IL-15RαSu/Anti-TF

In some embodiments, provided herein is a multi-chain chimeric polypeptide that includes: (a) a first chimeric polypeptide including: (i) a first target-binding domain that includes a human soluble IL-7 (e.g., a sequence that is at least 80%, at least 90%, at least 95%, or 100% identical to SEQ ID NO: 4); (ii) a soluble T cell receptor j-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a mouse soluble IL-21 (e.g., a sequence that is at least 80%, at least 90%, at least 95%, or 100% identical to SEQ ID NO: 124); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115);and (iii) an additional target-binding domain that includes an anti-human TF antigen-binding domain. In some embodiments, the anti-human TF antigen-binding domain is an anti-human TF diabody (e.g., a sequence that is at least 80%, at least 90%, at least 95%, or 100% identical to SEQ ID NO: 15). In some embodiments, the anti-human TF antigen-binding domain is an anti-human TFscFv (e.g., a sequence that is at least 80%, at least 90%, at least 95%, or 100% identical to SEQ ID NO: 131).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 152)
QGPDRLLIRLRHLIDIVEQLKIYENDLDPELLSAPQDVKGHCEHAAFAC
FQKAKLKPSNPGNNKTFIIDLVAQLRRRLPARRGGKKQKHIAKCPSCDS
YEKRTPKEFLERLKWLLQKMIHQHLSITCPPPMSVEHADIWVKSYSLYS
RERYICNSGFKRKAGTSSLTECVLNKATNVAHWTTPSLKCIRDIQMTQS
PASLSASVGDRVTITCLASQTIDTWLAWYLQKPGKSPQLLIYAATNLAD
GVPSRFSGSGSGTDFSFTISSLQPEDFATYYCQQVYSSPFTFGQGTKLE
IKGGGGSQIQLVQSGGEVKKPGASVRVSCKASGYSFTDYNVYWVRQSPG
KGLEWIGYIDPYNGITIYDQNFKGKATLTVDKSTSTAYMELSSLRSEDT
AVYFCARDVTTALDFWGQGTTVTVSSGGGGSGGGGSGGGGSDIQMTQSP
ASLSASVGDRVTITCLASQTIDTWLAWYLQKPGKSPQLLIYAATNLADG
VPSRFSGSGSGTDFSFTISSLQPEDFATYYCQQVYSSPFTFGQGTKLEI
KGGGGSQIQLVQSGGEVKKPGASVRVSCKASGYSFTDYNVYWVRQSPGK
GLEWIGYIDPYNGITIYDQNFKGKATLTVDKSTSTAYMELSSLRSEDTA
VYFCARDVTTALDFWGQGTTVTVSS.

In some embodiments, the second chimeric polypeptide further includes a signal sequence at its N-terminal end. In some embodiments the signal peptide sequence is MKWVTFISLLFLFSSAYS (SEQ ID NO: 80).
IL-7/hTRβC1/IL-15: IL-21/IL-15RαSu/Anti-PD-1 scFv

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble IL-21 (e.g., a sequence that is at least 80%, at least 90%, at least 95%, or 100% identical to SEQ ID NO: 5 or SEQ ID NO: 124); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human PD-1 scFv (e.g., a sequence that is at least 80%, at least 90%, at least 95%, or 100% identical to SEQ ID NO: 135).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 153)
MGVKVLFALICIAVAEAHKSSPQGPDRLLIRLRHLIDIVEQLKIYENDL
DPELLSAPQDVKGHCEHAAFACFQKAKLKPSNPGNNKTFIIDLVAQLRR
RLPARRGGKKQKHIAKCPSCDSYEKRTPKEFLERLKWLLQKMIHQHLSI
TCPPPMSVEHADIWVKSYSLYSRERYICNSGFKRKAGTSSLTECVLNKA
TNVAHWTTPSLKCIRGGGGSRTDVALTQTPVAQPVTLGDQASISCRSSQ
SLVHSNGRTYLEWYLQKPGQSPQLLIYKVSNRFSGVPDRFIGSGSGSDF
TLTISRVEPEDLGVYYCFQATHDPNTFGAGTKLELKGGGGSGGGGSGGG
GSQVQLQQSGAELVKPGSSVKISCKASGYTFTSHFIHWIKQQPGNGLEW
IGGIYPGDGDTEYNQQFNGKATLTADKSSSTAYMRLSSLTSEDSAVYFC
ATRVPSYWFFDFWGPGTMVTVSS.

In some embodiments, the second chimeric polypeptide further includes a signal sequence at its N-terminal end. In some embodiments the signal peptide sequence is MGVKVLFALICIAVAEA (SEQ ID NO: 137).

IL-7/hTRβC1/IL-15: mIL-21/IL-15RαSu/Anti-mPD-1 scFv

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble IL-21 (e.g., a sequence that is at least 80%, at least 90%, at least 95%, or 100% identical to SEQ ID NO: 5 or SEQ ID NO: 124); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-mouse PD-1 scFv (e.g., a sequence that is at least 80%, at least 90%, at least 95%, or 100% identical to SEQ ID NO: 136).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVEPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 154)
HKSSPQGPDRLLIRLRHLIDIVEQLKIYENDLDPELLSAPQDVKGHCEH
AAFACFQKAKLKPSNPGNNKTFIIDLVAQLRRRLPARRGGKKQKHIAKC
PSCDSYEKRTPKEFLERLKWLLQKMIHQHLSITCPPPMSVEHADIWVKS
YSLYSRERYICNSGFKRKAGTSSLTECVLNKATNVAHWTTPSLKCIRGG
GGSRTDVALTQTPVAQPVTLGDQASISCRSSQSLVHSNGRTYLEWYLQK
PGQSPQLLIYKVSNRFSGVPDRFIGSGSGSDFTLTISRVEPEDLGVYYC
FQATHDPNTFGAGTKLELKGGGGSGGGGSGGGGSQVQLQQSGAELVKPG
SSVKISCKASGYTFTSHFIHWIKQQPGNGLEWIGGIYPGDGDTEYNQQF
NGKATLTADKSSSTAYMRLSSLTSEDSAVYFCATRVPSYWFFDFWGPGT
MVTVSS.

In some embodiments, the second chimeric polypeptide further includes a signal sequence at its N-terminal end. In some embodiments the signal peptide sequence is MGVKVLFALICIAVAEA (SEQ ID NO: 137).

### IL-7/TRβC/IL-15: IL-21/IL-1SRαSu/Anti-PD-1(Diabody)

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble IL-21 (e.g., a sequence that is at least 80%, at least 90%, at least 95%, or 100% identical to SEQ ID NO: 5 or SEQ ID NO: 124); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-PD-1 diabody (e.g., a sequence that is at least 80%, at least 90%, at least 95%, or 100% identical to SEQ ID NO: 21 or SEQ ID NO: 134).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 146)
HKSSPQGPDRLLIRLRHLIDIVEQLKIYENDLDPELLSAPQDVKGHCEH
AAFACFQKAKLKPSNPGNNKTFIIDLVAQLRRRLPARRGGKKQKHIAKC
PSCDSYEKRTPKEFLERLKWLLQKMIHQHLSITCPPPMSVEHADIWVKS
YSLYSRERYICNSGFKRKAGTSSLTECVLNKATNVAHWTTPSLKCIRQV
QLQQSGAELVKPGSSVKISCKASGYTFTSHFIHWIKQQPGNGLEWIGGI
YPGDGDTEYNQQFNGKATLTADKSSSTAYMRLSSLTSEDSAVYFCATRV
PSYWFFDFWGPGTMVTVSSGGGGSRTDVALTQTPVAQPVTLGDQASISC
RSSQSLVHSNGRTYLEWYLQKPGQSPQLLIYKVSNRFSGVPDRFIGSGS
GSDFTLTISRVEPEDLGVYYCFQATHDPNTFGAGTKLELKGGGGSGGGG
SGGGGSQVQLQQSGAELVKPGSSVKISCKASGYTFTSHFIHWIKQQPGN
GLEWIGGIYPGDGDTEYNQQFNGKATLTADKSSSTAYMRLSSLTSEDSA
VYFCATRVPSYWFFDFWGPGTMVTVSSGGGGSRTDVALTQTPVAQPVTL
GDQASISCRSSQSLVHSNGRTYLEWYLQKPGQSPQLLIYKVSNRFSGVP
DRFIGSGSGSDFTLTISRVEPEDLGVYYCFQATHDPNTFGAGTKLELK.

In some embodiments, the second chimeric polypeptide further includes a signal sequence at its N-terminal end. In some embodiments the signal peptide sequence is MERTLVCLVVIFLGTVA (SEQ ID NO: 138).

IL-7/TRβC/IL-15: mIL-21/IL-15RαSu

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a mouse soluble IL-21 (e.g., a sequence that is at least 80%, at least 90%, at least 95%, or 100% identical to SEQ ID NO: 124); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 155)
HKSSPQGPDRLLIRLRHLIDIVEQLKIYENDLDPELLSAPQDVKGHCEH
AAFACFQKAKLKPSNPGNNKTFIIDLVAQLRRRLPARRGGKKQKHIAKC
PSCDSYEKRTPKEFLERLKWLLQKMIHQHLSITCPPPMSVEHADIWVKS
YSLYSRERYICNSGFKRKAGTSSLTECVLNKATNVAHWTTPSLKCIR.

In some embodiments, the second chimeric polypeptide further includes a signal sequence at its N-terminal end. In some embodiments the signal peptide sequence is MGVKVLFALICIAVAEA (SEQ ID NO: 137).

### Exemplary Multi-Chain Chimeric Polypeptides—Type C

In some embodiments of any of the multi-chain chimeric polypeptides described herein, the first target-binding domain and the second targeting-binding domain each independently bind specifically to a ligand of TGF-βRII.

In some examples of these multi-chain chimeric polypeptides, the first target-binding domain and the soluble TRβC domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble TRβC domain in the first chimeric polypeptide.

In some embodiments of these multi-chain chimeric polypeptides, the soluble TRβC domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble TRβC domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble T cell receptor β-chain constant region (TRC) domain can be any of the exemplary soluble T cell receptor β-chain constant region (TRC) domains described herein.

In some embodiments of these multi-chain chimeric polypeptides, the soluble T cell receptor β-chain constant region (TRC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these multi-chain chimeric polypeptides, the pair of affinity domains can be any of the exemplary pairs of affinity domains described herein. In some embodiments, the pair of affinity domains is a sushi domain from an alpha chain of human IL-15 receptor (IL-15Rα) and a soluble IL-15. In some embodiments, the second domain of the pair of affinity domains is a sushi domain from an alpha chain of human IL-15 receptor (IL-15Rα) and the first domain of the pair of affinity domains a soluble IL-15. In some embodiments, the first domain of the pair of affinity domains is a sushi domain from an alpha chain of human IL-15 receptor (IL-15Rα) and the second domain of the pair of affinity domains a soluble IL-15.

In some embodiments, the sushi domain from an alpha chain of IL-15 receptor (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 11.

In some embodiments, the sushi domain from an alpha chain of IL-15 receptor (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 115.

In some embodiments, a soluble IL-15 can include a sequence that is at least 70% identical, at least 75% identical, at least 80% identical, at least 85% identical, at least 90% identical, at least 95% identical, at least 99% identical, or 100% identical to SEQ ID NO: 13.

In some embodiments of these multi-chain chimeric polypeptides, the first target-binding domain is a soluble TGFβRII receptor, and the second target-binding domain is a soluble TGFβRII receptor.

In some embodiments of these multi-chain chimeric polypeptides, the soluble human TGFβRII includes a first sequence of soluble human TGFβRII and a second sequence of soluble human TGFβRII. In some embodiments of these multi-chain chimeric polypeptides, the soluble human TGFβRII includes a linker disposed between the first sequence of soluble human TGFβRII and the second sequence of soluble human TGFβRII. In some examples of these multi-chain chimeric polypeptides, the linker includes the sequence GGGGSGGGGSGGGGS (SEQ ID NO: 24).

A non-limiting example of a soluble TGFβRII receptor that binds specifically to a ligand of TGF-βRII can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7, and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 49, or SEQ ID NO: 50, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to: SEQ ID NO: 8, SEQ ID NO: 49, or SEQ ID NO: 50.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 51.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10 or SEQ ID NO: 52.In some embodiments, the second chimeric polypeptide further comprises one or more additional target-binding domains at the N-terminal end or the C-terminal end of the second chimeric polypeptide. In some embodiments, the second chimeric polypeptide further comprises one or more additional target-binding domains at the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

In some embodiments, the additional target-binding domain includes or is a soluble IL-7, an anti-tissue factor antigen-binding domain, or an anti-PD-1 antigen-binding domain.

In some embodiments, the additional target-binding domain includes or is an anti-tissue factor antigen-binding domain. In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53.

In some embodiments, anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 53.

In some embodiments, the anti-tissue factor antigen-binding domain can further include a linker sequence (e.g., any of the exemplary linker sequences described herein or known in the art) between the first sequence and the second sequence.

In some embodiments of any of the multi-chain chimeric polypeptides described herein, the additional target-binding domain includes or is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 15.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 16.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 129, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 130.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 131.

In some embodiments, the additional target-binding domain includes or is an anti-PD-1 antigen-binding domain. In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 64.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 21.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 22.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 134.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 135.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 136.

In some embodiments, the additional target-binding domain includes or is soluble IL-7. In some embodiments, a soluble IL-7 can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 4.

### TGFβRII/TRβC/IL-15: TGFβRII/IL-15RαSu

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTQKATLVCLATGFFPDHVELSWWVNGKEVHSGVSTDPQPLKEQ
PALNDSRYCLSSRLRVSATFWQNPRNHFRCQVQFYGLSENDEWTQDRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 142)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIR.

In some embodiments, the second chimeric polypeptide further includes a signal sequence at its N-terminal end. In some embodiments the signal peptide sequence is MKWVTFISLLFLFSSAYS (SEQ ID NO: 80).

In some embodiments, the first chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 157)
ATTCCTCCTCACGTGCAGAAAAGCGTGAACAACGACATGATAGTGACCG
ACAATAACGGCGCGGTCAAGTTCCCACAGCTCTGCAAATTTTGCGACGT
GAGATTTAGTACCTGCGACAATCAGAAATCCTGCATGAGCAATTGTAGC
ATCACTAGCATCTGTGAAAAGCCCCAAGAGGTCTGCGTAGCAGTGTGGA
GAAAAAATGACGAGAACATCACACTAGAGACCGTGTGCCATGACCCTAA
GCTGCCTTATCATGATTTCATCCTTGAAGATGCTGCTAGCCCTAAGTGC
ATTATGAAAGAGAAAAAGAAGCCTGGCGAAACCTTTTTCATGTGTAGCT
GTAGTAGCGACGAGTGCAACGATAACATCATCTTCTCGGAGGAGTATAA
TACATCGAATCCTGATGGCGGCGGCGGCAGCATCCCTCCTCATGTTCAG
AAGAGCGTGAATAACGACATGATTGTGACGGATAACAATGGCGCCGTAA
AGTTCCCTCAGCTGTGTAAGTTCTGCGATGTGCGATTTAGCACCTGCGA
CAATCAGAAGAGCTGTATGTCCAATTGTTCAATCACAAGCATCTGTGAG
AAGCCTCAAGAGGTGTGCGTGGCCGTTTGGAGAAAGAACGACGAGAACA
TTACCCTTGAGACCGTATGCCACGATCCTAAGCTGCCGTATCACGACTT
CATACTGGAGGATGCCGCTAGCCCTAAATGCATCATGAAAGAAAAAAAA
AAGCCTGGTGAGACATTTTTTATGTGTAGCTGCTCGTCTGACGAGTGCA
ATGACAATATCATTTTCAGCGAAGAATATAACACCTCAAACCCTGACGA
GGACCTGAACAAGGTGTTCCCACCCGAGGTCGCTGTGTTTGAGCCATCA
GAAGCAGAGATCTCCCACACCCAAAAGGCCACACTGGTGTGCCTGGCCA
CAGGCTTCTTCCCCGACCACGTGGAGCTGAGCTGGTGGGTGAATGGGAA
GGAGGTGCACAGTGGGGTCAGCACGGACCCGCAGCCCCTCAAGGAGCAG
CCCGCCCTCAATGACTCCAGATACTGCCTGAGCAGCCGCCTGAGGGTCT
CGGCCACCTTCTGGCAGAACCCCCGCAACCACTTCCGCTGTCAAGTCCA
GTTCTACGGGCTCTCGGAGAATGACGAGTGGACCCAGGATAGGGCCAAA
CCCGTCACCCAGATCGTCAGCGCCGAGGCCTGGGGTAGAGCAGACAACT
GGGTGAACGTGATCAGCGACCTGAAGAAGATCGAGGACCTGATTCAGAG
CATGCACATCGACGCCACCCTGTACACCGAGAGCGACGTGCACCCTAGC
TGCAAGGTGACCGCCATGAAGTGCTTCCTGCTGGAGCTGCAAGTGATCA
GCCTGGAGAGCGGCGACGCTAGCATCCACGACACCGTGGAGAACCTGAT
CATCCTGGCCAACAACAGCCTGAGCAGCAACGGCAACGTGACCGAGAGC
GGCTGCAAGGAGTGCGAGGAGCTGGAGGAGAAGAACATCAAGGAGTTCC
TGCAGAGCTTCGTGCACATCGTGCAGATGTTCATCAACACAAGCTAATG
A.

In some embodiments, the second chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 158)
ATGAAGTGGGTGACCTTCATCAGCCTGCTGTTCCTGTTCAGCAGCGCCT
ACAGCATCCCGCCACACGTGCAAAAGAGCGTGAACAACGACATGATCGT
TACGGACAACAATGGGGCCGTGAAGTTCCCTCAGTTATGCAAATTCTGT
GACGTGAGATTCAGCACGTGCGACAATCAGAAGAGCTGCATGTCCAATT
GCTCAATCACAAGCATCTGCGAGAAACCCCAAGAAGTTTGCGTGGCCGT
GTGGAGAAAGAATGACGAGAACATCACCCTGGAAACCGTGTGCCATGAT
CCTAAACTCCCTTACCATGACTTTATCTTGGAGGATGCTGCTAGCCCAA
AGTGCATCATGAAAGAGAAGAAGAAACCGGGGGAAACTTTTTTCATGTG
TTCATGCAGCTCTGACGAGTGTAATGATAACATTATCTTCAGCGAAGAG
TATAACACTAGCAATCCTGATGGAGGTGGCGGATCTATCCCCCCCCATG
TGCAAAAATCAGTGAACAATGACATGATCGTGACCGATAATAACGGGGC
GGTAAAGTTTCCTCAACTGTGTAAATTTTGCGACGTCAGATTCAGCACA
TGCGACAATCAAAAGTCCTGCATGAGCAACTGCAGCATCACCTCTATCT
GTGAGAAACCACAAGAAGTATGTGTGGCGGTATGGCGGAAGAACGATGA
AAATATCACGCTGGAGACCGTTTGCCATGATCCTAAGCTGCCATATCAC
GATTTCATCCTGGAGGACGCCGCTAGCCCTAAGTGTATCATGAAGGAGA
AGAAGAAGCCCGGTGAGACCTTTTTCATGTGCTCATGCAGCTCCGACGA
GTGCAACGACAACATCATCTTCAGCGAGGAGTACAATACAAGCAATCCT
GACATCACCTGCCCTCCTCCTATGAGCGTGGAGCACGCCGACATCTGGG
TGAAGAGCTACAGCCTGTACAGCAGAGAGAGATACATCTGCAACAGCGG
CTTCAAGAGAAAGGCCGGCACAAGCAGCCTGACCGAGTGCGTGCTGAAC
AAGGCCACCAACGTGGCCCACTGGACCACCCCTAGCCTGAAGTGCATCA
GATAA.

### TGFβRII/TRβC/IL-15: TGFβRII/IL-15RαSu/Anti-TF

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human TF antigen-binding domain. In some embodiments, the anti-human TF antigen-binding domain is an anti-human TF diabody (e.g., a sequence that is at least 80%, at least 90%, at least 95%, or 100% identical to SEQ ID NO: 15). In some embodiments, the anti-human TF antigen-binding domain is an anti-human TFscFv (e.g., a sequence that is at least 80%, at least 90%, at least 95%, or 100% identical to SEQ ID NO: 131).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTQKATLVCLATGFFPDHVELSWWVNGKEVHSGVSTDPQPLKEQ
PALNDSRYCLSSRLRVSATFWQNPRNHFRCQVQFYGLSENDEWTQDRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 144)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIRD
IQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFG
QGTKLEIKGGGGSQIQLVQSGGEVKKPGASVRVSCKASGYSFTDYNVYW
VRQSPGKGLEWIGYIDPYNGITIYDQNFKGKATLTVDKSTSTAYMELSS
LRSEDTAVYFCARDVTTALDFWGQGTTVTVSSGGGGSGGGGSGGGGSDI
QMTQSPASLSASVGDRVTITCLASQTIDTWLAWYLQKPGKSPQLLIYAA
TNLADGVPSRFSGSGSGTDFSFTISSLQPEDFATYYCQQVYSSPFTFGQ
GTKLEIKGGGGSQIQLVQSGGEVKKPGASVRVSCKASGYSFTDYNVYWV
RQSPGKGLEWIGYIDPYNGITIYDQNFKGKATLTVDKSTSTAYMELSSL
RSEDTAVYFCARDVTTALDFWGQGTTVTVSS.

In some embodiments, the second chimeric polypeptide further includes a signal sequence at its N-terminal end. In some embodiments the signal peptide sequence is MKWVTFISLLFLFS SAYS (SEQ ID NO: 80).
TGFβRII/TRβC/IL-15: TGFβRII/IL-15RαSu/Anti-Human PD-1 scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human PD-1 scFv (e.g., a sequence that is at least 80%, at least 90%, at least 95%, or 100% identical to SEQ ID NO: 135).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTQKATLVCLATGFFPDHVELSWWVNGKEVHSGVSTDPQPLKEQ
PALNDSRYCLSSRLRVSATFWQNPRNHFRCQVQFYGLSENDEWTQDRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 145)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIRG
GGGSEIVLTQSPATLSLSPGERATLSCRASKGVSTSGYSYLHWYQQKPG
QAPRLLIYLASYLESGVPARFSGSGSGTDFTLTISSLEPEDFAVYYCQH
SRDLPLTFGGGTKVEIKGGGGSGGGGSGGGGSQVQLVQSGVEVKKPGAS
VKVSCKASGYTFTNYYMYWVRQAPGQGLEWMGGINPSNGGTNFNEKFKN
RVTLTTDSSTTTAYMELKSLQFDDTAVYYCARRDYRFDMGFDYWGQGTT
VTVSS.

In some embodiments, the second chimeric polypeptide further includes a signal sequence at its N-terminal end. In some embodiments the signal peptide sequence is MKWVTFISLLFLFSSAYS (SEQ ID NO: 80).

### TGFβRII/TRβC/IL-15: TGFβRII/IL-15RαSu/IL-7

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a human soluble IL-7 (e.g., a sequence that is at least 80%, at least 90%, at least 95%, or 100% identical to SEQ ID NO: 4).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTQKATLVCLATGFFPDHVELSWWVNGKEVHSGVSTDPQPLKEQ
PALNDSRYCLSSRLRVSATFWQNPRNHFRCQVQFYGLSENDEWTQDRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 159)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIRD
CDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGR
KPAALGEAQPTKSLEENKSLKEQKKLNDLCFLKRLLQEIKTCWNKILMG
TKEH.

In some embodiments, the second chimeric polypeptide further includes a signal sequence at its N-terminal end. In some embodiments the signal peptide sequence is MKWVTFISLLFLFSSAYS (SEQ ID NO: 80).
TGFβRII/TRβC/IL-15: TGFβRII/IL-15RαSu/Anti-mPD-1 scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a soluble TGFβRII receptor (e.g., a sequence that is at least 80%, at least 90%, at least 95%, or 100% identical to SEQ ID NO: 9 or SEQ ID NO: 51); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-mouse PD-1 scFv (e.g., a sequence that is at least 80%, at least 90%, at least 95%, or 100% identical to SEQ ID NO: 136).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTQKATLVCLATGFFPDHVELSWWVNGKEVHSGVSTDPQPLKEQ
PALNDSRYCLSSRLRVSATFWQNPRNHFRCQVQFYGLSENDEWTQDRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 160)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDITCPPPMSVEHADIWVK
SYSLYSRERYICNSGFKRKAGTSSLTECVLNKATNVAHWTTPSLKCIRG
GGGSRTDVALTQTPVAQPVTLGDQASISCRSSQSLVHSNGRTYLEWYLQ
KPGQSPQLLIYKVSNRFSGVPDRFIGSGSGSDFTLTISRVEPEDLGVYY
CFQATHDPNTFGAGTKLELKGGGGSGGGGSGGGGSQVQLQQSGAELVKP
GSSVKISCKASGYTFTSHFIHWIKQQPGNGLEWIGGIYPGDGDTEYNQQ
FNGKATLTADKSSSTAYMRLSSLTSEDSAVYFCATRVPSYWFFDFWGPG
TMVTVSS.

In some embodiments, the second chimeric polypeptide further includes a signal sequence at its N-terminal end. In some embodiments the signal peptide sequence is MKWVTFISLLFLFSSAYS (SEQ ID NO: 80).

### Exemplary Multi-Chain Chimeric Polypeptides—Type D

In some embodiments of any of the multi-chain chimeric polypeptides described herein, the first target-binding domain and the second targeting-binding domain each independently bind specifically to a receptor of IL-7, to a ligand of TGF-βRII, or to CD3.

In some embodiments of these multi-chain chimeric polypeptides, the first target-binding domain and the second target-binding domain both bind specifically to a receptor of IL-7 or to CD3. In some embodiments of these multi-chain chimeric polypeptides, the first target-binding domain binds specifically to a receptor for IL-7, and the second target-binding domain binds specifically to CD3. In some embodiments, where the second target-binding domain binds specifically to CD3, the second target-binding domain can further bind specifically to tissue factor. In some embodiments of these multi-chain chimeric polypeptides, the first target-binding domain binds specifically to CD3, and the second target-binding domain bind specifically to a receptor for IL-7. In some embodiments, where the first target-binding domain binds specifically to CD3, the first target-binding domain can further bind specifically to tissue factor.

In some embodiments of these multi-chain chimeric polypeptides, the first target-binding domain and the second target-binding domain both bind specifically to a ligand of TGF-βRII or to CD3. In some embodiments of these multi-chain chimeric polypeptides, the first target-binding domain binds specifically to a ligand of TGF-βRII, and the second target-binding domain binds specifically to CD3. In some embodiments, where the second target-binding domain binds specifically to CD3, the second target-binding domain can further bind specifically to tissue factor. In some embodiments of these multi-chain chimeric polypeptides, the first target-binding domain binds specifically to CD3, and the second target-binding domain bind specifically to a ligand of TGF-βRII. In some embodiments, where first second target-binding domain binds specifically to CD3, the first target-binding domain can further bind specifically to tissue factor.

In some examples of these multi-chain chimeric polypeptides, the first target-binding domain and the soluble TRβC domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble TRβC domain in the first chimeric polypeptide.

In some embodiments of these multi-chain chimeric polypeptides, the soluble TRβC domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble TRβC domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble T cell receptor β-chain constant region (TRβC) domain can be any of the exemplary soluble T cell receptor β-chain constant region (TRβC) domains described herein.

In some embodiments of these multi-chain chimeric polypeptides, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these multi-chain chimeric polypeptides, the pair of affinity domains can be any of the exemplary pairs of affinity domains described herein. In some embodiments, the pair of affinity domains is a sushi domain from an alpha chain of human IL-15 receptor (IL-15Rα) and a soluble IL-15. In some embodiments, the second domain of the pair of affinity domains is a sushi domain from an alpha chain of human IL-15 receptor (IL-15Rα) and the first domain of the pair of affinity domains a soluble IL-15. In some embodiments, the first domain of the pair of affinity domains is a sushi domain from an alpha chain of human IL-15 receptor (IL-15Rα) and the second domain of the pair of affinity domains a soluble IL-15.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 11.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 115.

In some embodiments, a soluble IL-15 can include a sequence that is at least 70% identical, at least 75% identical, at least 80% identical, at least 85% identical, at least 90% identical, at least 95% identical, at least 99% identical, or 100% identical to SEQ ID NO: 13.

In some embodiments of these multi-chain chimeric polypeptides, one or both of the first target-binding domain and the second target-binding domain includes or is a soluble IL-7 or an anti-CD3 antigen-binding domain. In some embodiments, the first target-binding domain includes or is a soluble human IL-7, and the second target-binding domain includes or is an anti-CD3 antigen-binding domain. In some embodiments, where the second target-binding domain includes an anti-CD3 antigen-binding domain, the second target-binding domain further includes an anti-tissue factor antigen-binding domain. In some embodiments of these multi-chain chimeric polypeptides, the first target-binding domain is anti-CD3 antigen-binding domain, and the second target-binding domain is a soluble human IL-7. In some embodiments, where the first target-binding domain includes an anti-CD3 antigen-binding domain, the first target-binding domain further includes an anti-tissue factor antigen-binding domain.

In some embodiments of these multi-chain chimeric polypeptides, one or both of the first target-binding domain and the second target-binding domain includes or is a soluble IL-7 or an anti-CD3 antibody (e.g., a single chain CD3 (scCD3) antibody). In some embodiments of these multi-chain chimeric polypeptides, the first target-binding domain and the second target-binding domain are each independently a soluble IL-7 or an anti-CD3 antibody (e.g., a single chain CD3 (scCD3) antibody). In some embodiments of these multi-chain chimeric polypeptides, the first target-binding domain is a soluble human IL-7, and the second target-binding domain is a scCD3 antibody. In some embodiments, where the second target-binding domain includes a scCD3 antibody, the second target-binding domain further includes an anti-tissue factor antigen-binding domain. In some embodiments of these multi-chain chimeric polypeptides, the first target-binding domain is a scCD3 antibody, and the second target-binding domain is a soluble human IL-7. In some embodiments, where the first target-binding domain includes a scCD3 antibody, the first target-binding domain further includes an anti-tissue factor antigen-binding domain.

In some embodiments of these multi-chain chimeric polypeptides, one or both of the first target-binding domain and the second target-binding domain includes or is a soluble TGFβRII receptor or an anti-CD3 antigen-binding domain. In some embodiments, the first target-binding domain includes or is a soluble TGFβRII receptor, and the second target-binding domain includes or is an anti-CD3 antigen-binding domain. In some embodiments, where the second target-binding domain includes an anti-CD3 antigen-binding domain, the second target-binding domain further includes an anti-tissue factor antigen-binding domain. In some embodiments of these multi-chain chimeric polypeptides, the first target-binding domain is anti-CD3 antigen-binding domain, and the second target-binding domain is a soluble TGFβRII receptor. In some embodiments, where the first target-binding domain includes an anti-CD3 antigen-binding domain, the first target-binding domain further includes an anti-tissue factor antigen-binding domain.

In some embodiments of these multi-chain chimeric polypeptides, one or both of the first target-binding domain and the second target-binding domain is a soluble TGFβRII receptor or an anti-CD3 antibody (e.g., a single chain CD3 (scCD3) antibody). In some embodiments of these multi-chain chimeric polypeptides, the first target-binding domain and the second target-binding domain are each independently a soluble TGFβRII receptor or an anti-CD3 antibody (e.g., a single chain CD3 (scCD3) antibody). In some embodiments of these multi-chain chimeric polypeptides, the first target-binding domain is a soluble TGFβRII receptor, and the second target-binding domain is a scCD3 antibody. In some embodiments, where the second target-binding domain includes a scCD3 antibody, the second target-binding domain further includes an anti-tissue factor antigen-binding domain. In some embodiments of these multi-chain chimeric polypeptides, the first target-binding domain is a scCD3 antibody, and the second target-binding domain is a soluble TGFβRII receptor. In some embodiments, where the first target-binding domain includes a scCD3 antibody, the first target-binding domain further includes an anti-tissue factor antigen-binding domain.

A non-limiting example of an IL-7 that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

A non-limiting example of a soluble TGFβRII receptor that binds specifically to a ligand of TGF-βRII can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7, and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 51.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10 or SEQ ID NO: 52.

In some embodiments of these multi-chain chimeric polypeptides, the anti-CD3 antigen-binding domain includes or is a scCD3 antibody. A non-limiting example of a scCD3 antibody includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 17.

In some embodiments, a scCD3 antibody can be encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 18.

In some embodiments, a scCD3 antibody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 19.

In some embodiments, a scCD3 antibody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 20.

In some embodiments, the anti-CD3 antigen-binding domain includes or is an anti-CD3 scFv. In some embodiments, the anti-CD3 scFv can include a heavy chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 54 and/or a light chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 55.

In some embodiments, an anti-CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 58.

In some embodiments, the anti-CD3 scFv can be an anti-mouse CD3 scFv. In some embodiments, an anti-mouse CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 133.

In some embodiments of these multi-chain chimeric polypeptides, the anti-CD3 antigen-binding domain includes or is an anti-CD3 antibody. In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 60 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 61.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 62 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 63.

In some embodiments, a first target-binding domain and/or a second target-binding domain that includes both an anti-tissue factor domain and an anti-CD3 binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 117.

In some embodiments, the second chimeric polypeptide further comprises one or more additional target-binding domains at the N-terminal end or the C-terminal end of the second chimeric polypeptide. In some embodiments, the second chimeric polypeptide further comprises one or more additional target-binding domains at the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

In some embodiments, the additional target-binding domain includes or is an anti-tissue factor antigen-binding domain.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 53.

In some embodiments, an anti-tissue factor antigen-binding domain can further include a linker sequence (e.g., any of the exemplary linker sequences described herein or known in the art) between the first sequence and the second sequence.

In some embodiments, an anti-tissue factor antigen-binding domain includes or is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 15.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 16.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 129, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 130.

In some embodiments, an anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 131.

### TGFβRII/TRβC/IL-15: Anti-CD3scFv/IL-1SRαSu/Anti-TF

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-CD3scFv (e.g., a sequence that is at least 80%, at least 90%, at least 95%, or 100% identical to any one of SEQ ID NO: 17, SEQ ID NO: 19, and SEQ ID NO: 133); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human TF antigen-binding domain. In some embodiments, the anti-human TF antigen-binding domain is an anti-human TF diabody (e.g., a sequence that is at least 80%, at least 90%, at least 95%, or 100% identical to SEQ ID NO: 15). In some embodiments, the anti-human TF antigen-binding domain is an anti-human TFscFv (e.g., a sequence that is at least 80%, at least 90%, at least 95%, or 100% identical to SEQ ID NO: 131).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTQKATLVCLATGFFPDHVELSWWVNGKEVHSGVSTDPQPLKEQ
PALNDSRYCLSSRLRVSATFWQNPRNHFRCQVQFYGLSENDEWTQDRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 161)
DIQMTQSPSSLSASVGDRVTITCRASQDIRNYLNWYQQKPGKAPKLLIY
YTSRLESGVPSRFSGSGSGTDYTLTISSLQPEDFATYYCQQGNTLPWTF
GQGTKVEIKGGGGSGGGGSGGGGSEVQLVESGGGLVQPGGSLRLSCAAS
GYSFTGYTMNWVRQAPGKGLEWVALINPYKGVSTYNQKFKDRFTISVDK
SKNTAYLQMNSLRAEDTAVYYCARSGYYGDSDWYFDVWGQGTLVTVSSI
TCPPPMSVEHADIWVKSYSLYSRERYICNSGFKRKAGTSSLTECVLNKA
TNVAHWTTPSLKCIRDIQMTQSPASLSASVGDRVTITCLASQTIDTWLA
WYLQKPGKSPQLLIYAATNLADGVPSRFSGSGSGTDFSFTISSLQPEDF
ATYYCQQVYSSPFTFGQGTKLEIKGGGGSQIQLVQSGGEVKKPGASVRV
SCKASGYSFTDYNVYWVRQSPGKGLEWIGYIDPYNGITIYDQNFKGKAT
LTVDKSTSTAYMELSSLRSEDTAVYFCARDVTTALDFWGQGTTVTVSSG
GGGSGGGGSGGGGSDIQMTQSPASLSASVGDRVTITCLASQTIDTWLAW
YLQKPGKSPQLLIYAATNLADGVPSRFSGSGSGTDFSFTISSLQPEDFA
TYYCQQVYSSPFTFGQGTKLEIKGGGGSQIQLVQSGGEVKKPGASVRVS
CKASGYSFTDYNVYWVRQSPGKGLEWIGYIDPYNGITIYDQNFKGKATL
TVDKSTSTAYMELSSLRSEDTAVYFCARDVTTALDFWGQGTTVTVSS.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-CD3scFv/IL-1SRαSu/Anti-TF

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-CD3scFv (e.g., a sequence that is at least 80%, at least 90%, at least 95%, or 100% identical to any one of SEQ ID NO: 17, SEQ ID NO: 19, and SEQ ID NO: 133); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human TF antigen-binding domain. In some embodiments, the anti-human TF antigen-binding domain is an anti-human TF diabody (e.g., a sequence that is at least 80%, at least 90%, at least 95%, or 100% identical to SEQ ID NO: 15). In some embodiments, the anti-human TF antigen-binding domain is an anti-human TFscFv (e.g., a sequence that is at least 80%, at least 90%, at least 95%, or 100% identical to SEQ ID NO: 131).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVOMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 161)
DIQMTQSPSSLSASVGDRVTITCRASQDIRNYLNWYQQKPGKAPKLLIY
YTSRLESGVPSRFSGSGSGTDYTLTISSLOPEDFATYYCQQGNTLPWTF
GQGTKVEIKGGGGSGGGGSGGGGSEVOLVESGGGLVQPGGSLRLSCAAS
GYSFTGYTMNWVRQAPGKGLEWVALINPYKGVSTYNQKFKDRFTISVDK
SKNTAYLOMNSLRAEDTAVYYCARSGYYGDSDWYFDVWGOGTLVTVSSI
TCPPPMSVEHADIWVKSYSLYSRERYICNSGFKRKAGTSSLTECVLNKA
TNVAHWTTPSLKCIRDIQMTQSPASLSASVGDRVTITCLASQTIDTWLA
WYLOKPGKSPQLLIYAATNLADGVPSRESGSGSGTDFSFTISSLOPEDF
ATYYCQQVYSSPFTFGQGTKLEIKGGGGSQIQLVQSGGEVKKPGASVRV
SCKASGYSFTDYNVYWVRQSPGKGLEWIGYIDPYNGITIYDQNEKGKAT
LTVDKSTSTAYMELSSLRSEDTAVYFCARDVTTALDFWGQGTTVTVSSG
GGGSGGGGSGGGGSDIQMTQSPASLSASVGDRVTITCLASQTIDTWLAW
YLOKPGKSPQLLIYAATNLADGVPSRESGSGSGTDESFTISSLOPEDFA
TYYCQQVYSSPFTFGQGTKLEIKGGGGSQIQLVQSGGEVKKPGASVRVS
CKASGYSFTDYNVYWVRQSPGKGLEWIGYIDPYNGITIYDQNFKGKATL
TVDKSTSTAYMELSSLRSEDTAVYFCARDVTTALDFWGQGTTVTVSS.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-TFscFv(Humanized)-Anti-CD3scFv/IL-1SRαSu

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a humanized anti-human TFscFv and an anti-human CD3scFv (e.g., a sequence that is at least 80%, at least 90%, at least 95%, or 100% identical to SEQ ID NO: 117); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTOKATLVCLATGFFPDHVELS
WWVNGKEVHSGVSTDPQPLKEQPALNDSRYCLSSRLRVSATFWONPRNH
ERCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 162)
DIQMTQSPASLSASVGDRVTITCLASQTIDTWLAWYLOKPGKSPQLLIY
AATNLADGVPSRFSGSGSGTDESFTISSLOPEDFATYYCQQVYSSPFTF
GQGTKLEIKGGGGSGGGGSGGGGSQIQLVQSGGEVKKPGASVRVSCKAS
GYSFTDYNVYWVROSPGKGLEWIGYIDPYNGITIYDQNFKGKATLTVDK
STSTAYMELSSLRSEDTAVYFCARDVTTALDFWGQGTTVTVSSGGGGSD
IQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFG
QGTKVEIKGGGGSGGGGSGGGGSEVOLVESGGGLVQPGGSLRLSCAASG
YSFTGYTMNWVRQAPGKGLEWVALINPYKGVSTYNQKFKDRFTISVDKS
KNTAYLQMNSLRAEDTAVYYCARSGYYGDSDWYFDVWGQGTLVTVSSGG
GGSITCPPPMSVEHADIWVKSYSLYSRERYICNSGFKRKAGTSSLTECV
LNKATNVAHWTTPSLKCIR.

In some embodiments, the second chimeric polypeptide further includes a signal sequence at its N-terminal end. In some embodiments the signal peptide sequence is MGWSCIILFLVATATGVHS (SEQ ID NO: 139).

### TGFβRII/TRβC/IL-15: Anti-TFscFv(Humanized)-Anti-CD3scFv/IL-1SRαSu

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a humanized anti-human TFscFv and an anti-human CD3scFv (e.g., a sequence that is at least 80%, at least 90%, at least 95%, or 100% identical to SEQ ID NO: 117); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVO
KSVNNDMIVTDNNGAVKFPQLCKFCDVRESTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTOKATLVCLATGFFPDHVELSWWVNGKEVHSGVSTDPQPLKEQ
PALNDSRYCLSSRLRVSATFWQNPRNHFRCQVQFYGLSENDEWTQDRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVOMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 162)
DIQMTQSPASLSASVGDRVTITCLASQTIDTWLAWYLOKPGKSPOLLIY
AATNLADGVPSRFSGSGSGTDFSFTISSLOPEDFATYYCQQVYSSPFTF
GQGTKLEIKGGGGSGGGGSGGGGSQIQLVQSGGEVKKPGASVRVSCKAS
GYSFTDYNVYWVROSPGKGLEWIGYIDPYNGITIYDQNFKGKATLTVDK
STSTAYMELSSLRSEDTAVYFCARDVTTALDFWGQGTTVTVSSGGGGSD
IQMTQSPSSLSASVGDRVTITCRASQDIRNYLNWYQQKPGKAPKLLIYY
TSRLESGVPSRFSGSGSGTDYTLTISSLOPEDFATYYCQQGNTLPWTFG
QGTKVEIKGGGGSGGGGSGGGGSEVOLVESGGGLVQPGGSLRLSCAASG
YSFTGYTMNWVRQAPGKGLEWVALINPYKGVSTYNQKFKDRFTISVDKS
KNTAYLOMNSLRAEDTAVYYCARSGYYGDSDWYFDVWGQGTLVTVSSGG
GGSITCPPPMSVEHADIWVKSYSLYSRERYICNSGFKRKAGTSSLTECV
LNKATNVAHWTTPSLKCIR.

In some embodiments, the second chimeric polypeptide further includes a signal sequence at its N-terminal end. In some embodiments the signal peptide sequence is MGWSCIILFLVATATGVHS (SEQ ID NO: 139).

### Exemplary Multi-Chain Chimeric Polypeptides—Type E

In some embodiments of any of the multi-chain chimeric polypeptides described herein, the first target-binding domain and the second targeting-binding domain each independently bind specifically to a receptor of IL-7, to a ligand of TGF-βRII, or to tissue factor.

In some embodiments of these multi-chain chimeric polypeptides, the first target-binding domain and the second target-binding domain both bind specifically to a receptor of IL-7 or to an tissue factor. In some embodiments of these multi-chain chimeric polypeptides, the first target-binding domain binds specifically to a receptor for IL-7, and the second target-binding domain binds specifically to tissue factor. In some embodiments of these multi-chain chimeric polypeptides, the first target-binding domain binds specifically to tissue factor, and the second target-binding domain bind specifically to a receptor for IL-7.

In some embodiments of these multi-chain chimeric polypeptides, the first target-binding domain and the second target-binding domain both bind specifically to a ligand of TGF-βRII or to tissue factor. In some embodiments of these multi-chain chimeric polypeptides, the first target-binding domain binds specifically to a ligand of TGF-βRII, and the second target-binding domain binds specifically to tissue factor. In some embodiments of these multi-chain chimeric polypeptides, the first target-binding domain binds specifically to tissue factor, and the second target-binding domain bind specifically to a ligand of TGF-βRII.

In some examples of these multi-chain chimeric polypeptides, the first target-binding domain and the soluble TRβC domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble TRβC domain in the first chimeric polypeptide.

In some embodiments of these multi-chain chimeric polypeptides, the soluble TRβC domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble TRβC domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble T cell receptor β-chain constant region (TRβC) domain can be any of the exemplary soluble T cell receptor β-chain constant region (TRβC) domains described herein.

In some embodiments of these multi-chain chimeric polypeptides, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these multi-chain chimeric polypeptides, the pair of affinity domains can be any of the exemplary pairs of affinity domains described herein. In some embodiments, the pair of affinity domains is a sushi domain from an alpha chain of human IL-15 receptor (IL-15Rα) and a soluble IL-15. In some embodiments, the second domain of the pair of affinity domains is a sushi domain from an alpha chain of human IL-15 receptor (IL-15Rα) and the first domain of the pair of affinity domains a soluble IL-15. In some embodiments, the first domain of the pair of affinity domains is a sushi domain from an alpha chain of human IL-15 receptor (IL-15Rα) and the second domain of the pair of affinity domains a soluble IL-15.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 11.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 115.

In some embodiments, a soluble IL-15 can include a sequence that is at least 70% identical, at least 75% identical, at least 80% identical, at least 85% identical, at least 90% identical, at least 95% identical, at least 99% identical, or 100% identical to SEQ ID NO: 13.

In some embodiments of these multi-chain chimeric polypeptides, one or both of the first target-binding domain and the second target-binding domain is a soluble IL-7 or an anti-tissue factor antigen-binding domain. In some embodiments of these multi-chain chimeric polypeptides, the first target-binding domain and the second target-binding domain are each independently a soluble IL-7 or an anti-tissue factor antigen-binding domain. In some embodiments of these multi-chain chimeric polypeptides, the first target-binding domain is a soluble IL-7, and the second target-binding domain is an anti-tissue factor antigen-binding domain. In some embodiments of these multi-chain chimeric polypeptides, the first target-binding domain is an anti-tissue factor antigen-binding domain, and the second target-binding domain is a soluble IL-7.

In some embodiments of these multi-chain chimeric polypeptides, one or both of the first target-binding domain and the second target-binding domain is a soluble IL-7 or a tissue factor (TF) antibody (e.g., a single chain TF antibody). In some embodiments of these multi-chain chimeric polypeptides, the first target-binding domain and the second target-binding domain are each independently a soluble IL-7 or an anti-TF antibody (e.g., a single chain TF antibody).

In some embodiments of these multi-chain chimeric polypeptides, the first target-binding domain is a soluble human IL-7, and the second target-binding domain is a single chain TF antibody. In some embodiments of these multi-chain chimeric polypeptides, the first target-binding domain is a single chain TF antibody, and the second target-binding domain is a soluble human IL-7. In some embodiments of these multi-chain chimeric polypeptides, the first target-binding domain is a soluble IL-7, and the second target-binding domain is an anti-TF diabody. In some embodiments of these multi-chain chimeric polypeptides, the first target-binding domain is a soluble IL-7, and the second target-binding domain is an anti-TF antibody in single chain format.

In some embodiments of these multi-chain chimeric polypeptides, one or both of the first target-binding domain and the second target-binding domain is a soluble TGFβRII receptor or an anti-tissue factor antigen-binding domain. In some embodiments of these multi-chain chimeric polypeptides, the first target-binding domain and the second target-binding domain are each independently a soluble TGFβRII receptor or an anti-tissue factor antigen-binding domain. In some embodiments of these multi-chain chimeric polypeptides, the first target-binding domain is a soluble TGFβRII receptor, and the second target-binding domain is an anti-tissue factor antigen-binding domain. In some embodiments of these multi-chain chimeric polypeptides, the first target-binding domain is an anti-tissue factor antigen-binding domain, and the second target-binding domain is a soluble TGFβRII receptor.

In some embodiments of these multi-chain chimeric polypeptides, one or both of the first target-binding domain and the second target-binding domain a soluble TGFβRII receptor or a tissue factor (TF) antibody (e.g., a single chain TF antibody). In some embodiments of these multi-chain chimeric polypeptides, the first target-binding domain and the second target-binding domain are each independently a soluble TGFβRII receptor or an anti-TF antibody (e.g., a single chain TF antibody).

In some embodiments of these multi-chain chimeric polypeptides, the first target-binding domain is a soluble TGFβRII receptor, and the second target-binding domain is a single chain anti-TF antibody. In some embodiments of these multi-chain chimeric polypeptides, the first target-binding domain is a single chain anti-TF antibody, and the second target-binding domain is a soluble TGFβRII receptor.

In some embodiments of these multi-chain chimeric polypeptides, the first target-binding domain is a soluble TGFβRII receptor, and the second target-binding domain is an anti-TF diabody. In some embodiments of these multi-chain chimeric polypeptides, the first target-binding domain is a soluble TGFβRII receptor, and the second target-binding domain is an anti-TF antibody in single chain format.

A non-limiting example of an IL-7 that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

A non-limiting example of a soluble TGFβRII receptor that binds specifically to a ligand of TGF-βRII can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7, and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 49, or SEQ ID NO: 50, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to: SEQ ID NO: 8, SEQ ID NO: 49, or SEQ ID NO: 50.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 51.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10 or SEQ ID NO: 52.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 53.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 15.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 16.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 129, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 130.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 131.

In some embodiments, the second chimeric polypeptide further comprises one or more additional target-binding domains at the N-terminal end or the C-terminal end of the second chimeric polypeptide. In some embodiments, the second chimeric polypeptide further comprises one or more additional target-binding domains at the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

In some embodiments, the additional target-binding domain includes or is an anti-CD3 antigen-binding domain.

In some embodiments, the additional target-binding domain includes or is a mouse anti-CD3 antigen-binding domain. In some embodiments, the additional target-binding domain includes or is a human anti-CD3 antigen-binding domain.

In some embodiments, the additional target-binding domain includes or is a single-chain anti-CD3 antibody. A non-limiting example of a scCD3 antibody includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 17.

In some embodiments, a scCD3 antibody can be encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 18.

In some embodiments, a scCD3 antibody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 19.

In some embodiments, a scCD3 antibody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 20.

In some embodiments, the additional target-binding domain includes or is an anti-CD3 scFv. In some embodiments, the anti-CD3 scFv can include a heavy chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 54 and/or a light chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 55.

In some embodiments, an anti-CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 58.

In some embodiments, the anti-CD3 scFv can be an anti-mouse CD3 scFv. In some embodiments, an anti-mouse CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 133.

In some embodiments, the additional target-binding domain includes or is an anti-CD3 antibody. In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 60 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 61.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 62 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 63.

In some embodiments, the additional target-binding domain includes or is a soluble IL-21. A non-limiting example of an IL-21 that binds specifically to an IL-21 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 5.

In some embodiments, an IL-21 that binds specifically to an IL-21 receptor can be encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 6 or 23.

In some embodiments, the additional target-binding domain includes or is a soluble human TL-21. In some embodiments, the additional target-binding domain includes or is a soluble mouse IL-21.

In some embodiments, the additional target-binding domain includes or is an anti-PD-1 antigen-binding domain. In some embodiments, the additional target-binding domain includes or is a human anti-PD-1 antigen-binding domain. In some embodiments, the additional target-binding domain includes or is a mouse anti-PD-1 antigen-binding domain. In some embodiments, the additional target-binding domain includes or is an anti-PD-1 single chain antibody.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 64.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 21.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 22.

In some embodiments, the anti-PD-1 antigen-binding domain is a mouse anti-PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 134.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 135.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 136.

In some embodiments, the additional target-binding domain includes or is an anti-CD16 antigen-binding domain.

### TGFβRII/TRβC/IL-15: Anti-TF(Diabody)/IL-1SRαSu/Anti-Human CD3scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TF diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-CD3 antigen-binding domain (e.g., a sequence that is at least 80%, at least 90%, at least 95%, or 100% identical to any one of SEQ ID NO: 19).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVO
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTOKATLVCLATGFFPDHVELSWWVNGKEVHSGVSTDPQPLKEQ
PALNDSRYCLSSRLRVSATFWQNPRNHFRCQVQFYGLSENDEWTQDRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVOMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 163)
DIQMTQSPASLSASVGDRVTITCLASQTIDTWLAWYLQKPGKSPOLLIY
AATNLADGVPSRFSGSGSGTDESFTISSLOPEDFATYYCQQVYSSPFTF
GQGTKLEIKGGGGSQIQLVQSGGEVKKPGASVRVSCKASGYSFTDYNVY
WVROSPGKGLEWIGYIDPYNGITIYDONFKGKATLTVDKSTSTAYMELS
SLRSEDTAVYFCARDVTTALDFWGQGTTVTVSSGGGGSGGGGSGGGGSD
IOMTOSPASLSASVGDRVTITCLASQTIDTWLAWYLOKPGKSPOLLIYA
ATNLADGVPSRESGSGSGTDFSFTISSLOPEDFATYYCQQVYSSPFTFG
QGTKLEIKGGGGSQIQLVQSGGEVKKPGASVRVSCKASGYSFTDYNVYW
VROSPGKGLEWIGYIDPYNGITIYDONFKGKATLTVDKSTSTAYMELSS
LRSEDTAVYFCARDVTTALDFWGOGTTVTVSSITCPPPMSVEHADIWVK
SYSLYSRERYICNSGFKRKAGTSSLTECVLNKATNVAHWTTPSLKCIRD
IKLQQSGAELARPGASVKMSCKTSGYTFTRYTMHWVKORPGQGLEWIGY
INPSRGYTNYNQKFKDKATLTTDKSSSTAYMOLSSLTSEDSAVYYCARY
YDDHYCLDYWGQGTTLTVSSVEGGSGGSGGSGGSGGVDDIQLTQSPAIM
SASPGEKVTMTCRASSSVSYMNWYQQKSGTSPKRWIYDTSKVASGVPYR
FSGSGSGTSYSLTISSMEAEDAATYYCQQWSSNPLTFGAGTKLELK.

In some embodiments, the second chimeric polypeptide further includes a signal sequence at its N-terminal end. In some embodiments the signal peptide sequence is MGWSCIILFLVATATGVHS (SEQ ID NO: 139).

### TGFβRII/TRβC/IL-15: Anti-TF(Diabody)/IL-1SRαSu/Anti-Mouse CD3scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a humanized anti-human tissue factor diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-mouse CD3scFv (e.g., a sequence that is at least 80%, at least 90%, at least 95%, or 100% identical to any one of SEQ ID NO: 133).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVO
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTOKATLVCLATGFFPDHVELSWWVNGKEVHSGVSTDPOPLKEQ
PALNDSRYCLSSRLRVSATFWQNPRNHFRCQVQFYGLSENDEWTODRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVOMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 164)
DIQMTQSPASLSASVGDRVTITCLASQTIDTWLAWYLQKPGKSPOLLIY
AATNLADGVPSRFSGSGSGTDFSFTISSLOPEDFATYYCQQVYSSPFTF
GQGTKLEIKGGGGSQIQLVQSGGEVKKPGASVRVSCKASGYSFTDYNVY
WVRWSPGKGLEWIGYIDPYNGITIYDQNFKGKATLTVDKSTSTAYMELS
SLRSEDTAVYFCARDVTTALDFWGQGTTVTVSSGGGGSGGGGSGGGGSD
IQMTQSPASLSASVGDRVTITCLASQTIDTWLAWYLWKPGKSPWLLIYA
ATNLADGVPSRFSGSGSGTDESFTISSLQPEDFATYYCQQVYSSPFTFG
QGTKLEIKGGGGSQIQLVQSGGEVKKPGASVRVSCKASGYSFTDYNVYW
VRWSPGKGLEWIGYIDPYNGITIYDWNFKGKATLTVDKSTSTAYMELSS
LRSEDTAVYFCARDVTTALDFWGQGTTVTVSSITCPPPMSVEHADIWVK
SYSLYSRERYICNSGFKRKAGTSSLTECVLNKATNVAHWTTPSLKCIRD
IQMTQSPSSLPASLGDRVTINCQASQDISNYLNWYQQKPGKAPKLLIYY
TNKLADGVPSRFSGSGSGRDSSFTISSLESEDIGSYYCQQYYNYPWTFG
PGTKLEIKGGGGSGGGGSGGGGSEVWLVESGGGLVQPGKSLKLSCEASG
FTFSGYGMHWVRQAPGRGLESVAYITSSSINIKYADAVKGRFTVSRDNA
KNLLFLQMNILKSEDTAMYYCARFDWDKNYWGQGTMVTVSS.

In some embodiments, the second chimeric polypeptide further includes a signal sequence at its N-terminal end. In some embodiments the signal peptide sequence is MGWSCIILFLVATATGVHS (SEQ ID NO: 139).

### TGFβRII/TRβC/IL-15: Anti-TF(Diabody)/IL-1SRαSu/Anti-Human CD3scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TF diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human CD3scFv (e.g., a sequence that is at least 80%, at least 90%, at least 95%, or 100% identical to any one of SEQ ID NO: 17).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSIPPHVQ
KSVNNDMIVTDNNGAVKFPQLCKFCDVRFSTCDNQKSCMSNCSITSICE
KPQEVCVAVWRKNDENITLETVCHDPKLPYHDFILEDAASPKCIMKEKK
KPGETFFMCSCSSDECNDNIIFSEEYNTSNPDEDLNKVFPPEVAVFEPS
EAEISHTQKATLVCLATGFFPDHVELSWWVNGKEVHSGVSTDPQPLKEQ
PALNDSRYCLSSRLRVSATFWQNPRNHERCQVQFYGLSENDEWTQDRAK
PVTQIVSAEAWGRADNWVNVISDLKKIEDLIQSMHIDATLYTESDVHPS
CKVTAMKCFLLELQVISLESGDASIHDTVENLIILANNSLSSNGNVTES
GCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 165)
DIQMTQSPASLSASVGDRVTITCLASQTIDTWLAWYLQKPGKSPOLLIY
AATNLADGVPSRFSGSGSGTDESFTISSLQPEDFATYYCQQVYSSPFTF
GQGTKLEIKGGGGSQIQLVQSGGEVKKPGASVRVSCKASGYSFTDYNVY
WVRQSPGKGLEWIGYIDPYNGITIYDQNFKGKATLTVDKSTSTAYMELS
SLRSEDTAVYFCARDVTTALDFWGQGTTVTVSSGGGGSGGGGSGGGGSD
IQMTQSPASLSASVGDRVTITCLASQTIDTWLAWYLQKPGKSPQLLIYA
ATNLADGVPSRESGSGSGTDFSFTISSLQPEDFATYYCQQVYSSPFTFG
QGTKLEIKGGGGSQIQLVQSGGEVKKPGASVRVSCKASGYSFTDYNVYW
VRQSPGKGLEWIGYIDPYNGITIYDQNFKGKATLTVDKSTSTAYMELSS
IWVKSYSLYSRERYICNSGFKRKAGTSSLTECVLNKATNVAHWTTPSLK
LRSEDTAVYFCARDVITALDFWGQGTTVTVSSITCPPPMSVEHADCIRD
IQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFG
QGTKVEIKGGGGSGGGGSGGGGSEVQLVESGGGLVQPGGSLRLSCAASG
YSFTGYTMNWVRQAPGKGLEWVALINPYKGVSTYNQKFKDRFTISVDKS
KNTAYLQMNSLRAEDTAVYYCARSGYYGDSDWYFDVWGQGTLVTVSS.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-TF(Diabody)/IL-1SRαSu/Anti-Human CD3scFv

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TF diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human CD3scFv (e.g., a sequence that is at least 80%, at least 90%, at least 95%, or 100% identical to any one of SEQ ID NO: 19).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
ERCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 163)
DIQMTQSPASLSASVGDRVTITCLASQTIDTWLAWYLQKPGKSPQLLIY
AATNLADGVPSRFSGSGSGTDFSFTISSLQPEDFATYYCQQVYSSPFTF
GQGTKLEIKGGGGSQIQLVQSGGEVKKPGASVRVSCKASGYSFTDYNVY
WVRQSPGKGLEWIGYIDPYNGITIYDQNFKGKATLTVDKSTSTAYMELS
SLRSEDTAVYFCARDVTTALDFWGQGTTVTVSSGGGGSGGGGSGGGGSD
IQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFG
QGTKLEIKGGGGSQIQLVQSGGEVKKPGASVRVSCKASGYSFTDYNVYW
VRQSPGKGLEWIGYIDPYNGITIYDQNFKGKATLTVDKSTSTAYMELSS
LRSEDTAVYFCARDVITALDFWGQGTTVTVSSITCPPPMSVEHADIWVK
SYSLYSRERYICNSGFKRKAGTSSLTECVLNKATNVAHWTTPSLKCIRD
IKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARY
YDDHYCLDYWGQGTTLTVSSVEGGSGGSGGSGGSGGVDDIQLTQSPAIM
SASPGEKVTMTCRASSSVSYMNWYQQKSGTSPKRWIYDTSKVASGVPYR
FSGSGSGTSYSLTISSMEAEDAATYYCQQWSSNPLTFGAGTKLELK.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-TF(Diabody)/IL-1SRαSu/Anti-Mouse CD3scFv

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human tissue factor diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-mouse CD3scFv (e.g., a sequence that is at least 80%, at least 90%, at least 95%, or 100% identical to any one of SEQ ID NO: 133).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 164)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSQIQLVQSGGEVKKPGASVRVSCKASGYSFTDYNVYWVR
OSPGKGLEWIGYIDPYNGITIYDQNFKGKATLTVDKSTSTAYMELSSLRS
EDTAVYFCARDVTTALDFWGQGTTVTVSSGGGGSGGGGSGGGGSDIQMTQ
SPASLSASVGDRVTITCLASQTIDTWLAWYLQKPGKSPQLLIYAATNLAD
GVPSRFSGSGSGTDFSFTISSLQPEDFATYYCQQVYSSPFTFGQGTKLEI
KGGGGSQIQLVQSGGEVKKPGASVRVSCKASGYSFTDYNVYWVRQSPGKG
LEWIGYIDPYNGITIYDQNFKGKATLTVDKSTSTAYMELSSLRSEDTAVY
FCARDVTTALDFWGQGTTVTVSSITCPPPMSVEHADIWVKSYSLYSRERY
ICNSGFKRKAGTSSLTECVLNKATNVAHWTTPSLKCIRDIQMTQSPSSLP
ASLGDRVTINCQASQDISNYLNWYQQKPGKAPKLLIYYTNKLADGVPSRF
SGSGSGRDSSFTISSLESEDIGSYYCQQYYNYPWTFGPGTKLEIKGGGGS
GGGGSGGGGSEVQLVESGGGLVQPGKSLKLSCEASGFTFSGYGMHWVRQA
PGRGLESVAYITSSSINIKYADAVKGRFTVSRDNAKNLLFLOMNILKSED
TAMYYCARFDWDKNYWGQGTMVTVSS.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-TF(Diabody)/IL-1SRαSu/Anti-Human CD3scFv

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human tissue factor diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human CD3scFv (e.g., a sequence that is at least 80%, at least 90%, at least 95%, or 100% identical to any one of SEQ ID NO: 17).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EHEDLNKVFPPEVAVFEPSEAEISHTQKATLVCLATGFFPDHVELSWWVN
GKEVHSGVSTDPQPLKEQPALNDSRYCLSSRLRVSATFWQNPRNHFRCQV
QFYGLSENDEWTQDRAKPVTQIVSAEAWGRADNWVNVISDLKKIEDLIQS
MHIDATLYTESDVHPSCKVTAMKCFLLELQVISLESGDASIHDTVENLII
LANNSLSSNGNVTESGCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 165)
DIQMTQSPASLSASVGDRVTITCLASQTIDTWLAWYLQKPGKSPQLLIYA
ATNLADGVPSRFSGSGSGTDESFTISSLQPEDFATYYCQQVYSSPFTFGQ
GTKLEIKGGGGSQIQLVQSGGEVKKPGASVRVSCKASGYSFTDYNVYWVR
QSPGKGLEWIGYIDPYNGITIYDQNFKGKATLTVDKSTSTAYMELSSLRS
EDTAVYFCARDVTTALDFWGQGTTVTVSSGGGGSGGGGSGGGGSDIQMTQ
SPASLSASVGDRVTITCLASQTIDTWLAWYLQKPGKSPQLLIYAATNLAD
GVPSRFSGSGSGTDFSFTISSLQPEDFATYYCQQVYSSPFTFGQGTKLEI
KGGGGSQIQLVQSGGEVKKPGASVRVSCKASGYSFTDYNVYWVRQSPGKG
LEWIGYIDPYNGITIYDQNFKGKATLTVDKSTSTAYMELSSLRSEDTAVY
FCARDVTTALDFWGQGTTVTVSSITCPPPMSVEHADIWVKSYSLYSRERY
ICNSGFKRKAGTSSLTECVLNKATNVAHWTTPSLKCIRDIQMTQSPSSLS
ASVGDRVTITCRASQDIRNYLNWYQQKPGKAPKLLIYYTSRLESGVPSRF
SGSGSGTDYTLTISSLQPEDFATYYCQQGNTLPWTFGQGTKVEIKGGGGS
GGGGSGGGGSEVQLVESGGGLVQPGGSLRLSCAASGYSFTGYTMNWVRQA
PGKGLEWVALINPYKGVSTYNQKFKDRFTISVDKSKNTAYLQMNSLRAED
TAVYYCARSGYYGDSDWYFDVWGQGTLVTVSS.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-TFscFv/IL-1SRαSu/Anti-Human CD3scFv

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TFscFv (e.g., a sequence that is at least 80%, at least 90%, at least 95%, or 100% identical to SEQ ID NO: 131); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human CD3scFv (e.g., a sequence that is at least 80%, at least 90%, at least 95%, or 100% identical to any one of SEQ ID NO: 17 OR SEQ ID NO: 19).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EHEDLNKVFPPEVAVFEPSEAEISHTQKATLVCLATGFFPDHVELSWWVN
GKEVHSGVSTDPQPLKEQPALNDSRYCLSSRLRVSATFWQNPRNHERCQV
QFYGLSENDEWTQDRAKPVTQIVSAEAWGRADNWVNVISDLKKIEDLIQS
MHIDATLYTESDVHPSCKVTAMKCFLLELQVISLESGDASIHDTVENLII
LANNSLSSNGNVTESGCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 166)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSGGGGSGGGGSQIQLVQSGGEVKKPGASVRVSCKASGYS
FTDYNVYWVRQSPGKGLEWIGYIDPYNGITIYDQNFKGKATLTVDKSTST
AYMELSSLRSEDTAVYFCARDVTTALDFWGQGTTVTVSSGGGGSITCPPP
MSVEHADIWVKSYSLYSRERYICNSGFKRKAGTSSLTECVLNKATNVAHW
TTPSLKCIRDPALVHQRDIQMTQSPSSLSASVGDRVTITCRASQDIRNYL
NWYQQKPGKAPKLLIYYTSRLESGVPSRFSGSGSGTDYTLTISSLQPEDF
ATYYCQQGNTLPWTFGQGTKVEIKGGGGSGGGGSGGGGSEVQLVESGGGL
VQPGGSLRLSCAASGYSFTGYTMNWVRQAPGKGLEWVALINPYKGVSTYN
QKFKDRFTISVDKSKNTAYLQMNSLRAEDTAVYYCARSGYYGDSDWYFDV
WGQGTLVTVSS.

In some embodiments, the second chimeric polypeptide further includes a signal sequence at its N-terminal end. In some embodiments the signal peptide sequence is MGWSCIILFLVATATGVHS (SEQ ID NO: 139).

In some embodiments, the first chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 167)
GATTGTGATATTGAAGGTAAAGATGGCAAACAATATGAGAGTGTTCTAAT
GGTCAGCATCGATCAATTATTGGACAGCATGAAAGAAATTGGTAGCAATT
GCCTGAATAATGAATTTAACTTTTTTAAAAGACATATCTGTGATGCTAAT
AAGGAAGGTATGTTTTTATTCCGTGCTGCTCGCAAGTTGAGGCAATTTCT
TAAAATGAATAGCACTGGTGATTTTGATCTCCACTTATTAAAAGTTTCAG
AAGGCACAACAATACTGTTGAACTGCACTGGCCAGGTTAAAGGAAGAAAA
CCAGCTGCCCTGGGTGAAGCCCAACCAACAAAGAGTTTGGAAGAAAATAA
ATCTTTAAAGGAACAGAAAAAACTGAATGACTTGTGTTTCCTAAAGAGAC
TATTACAAGAGATAAAAACTTGTTGGAATAAAATTTTGATGGGCACTAAA
GAACACGAGGACCTGAACAAGGTGTTCCCACCCGAGGTCGCTGTGTTTGA
GCCATCAGAAGCAGAGATCTCCCACACCCAAAAGGCCACACTGGTGTGCC
TGGCCACAGGCTTCTTCCCCGACCACGTGGAGCTGAGCTGGTGGGTGAAT
GGGAAGGAGGTGCACAGTGGGGTCAGCACGGACCCGCAGCCCCTCAAGGA
GCAGCCCGCCCTCAATGACTCCAGATACTGCCTGAGCAGCCGCCTGAGGG
TCTCGGCCACCTTCTGGCAGAACCCCCGCAACCACTTCCGCTGTCAAGTC
CAGTTCTACGGGCTCTCGGAGAATGACGAGTGGACCCAGGATAGGGCCAA
ACCCGTCACCCAGATCGTCAGCGCCGAGGCCTGGGGTAGAGCAGACAACT
GGGTGAACGTGATCAGCGACCTGAAGAAGATCGAGGACCTGATTCAGAGC
ATGCACATCGACGCCACCCTGTACACCGAGAGCGACGTGCACCCTAGCTG
CAAGGTGACCGCCATGAAGTGCTTCCTGCTGGAGCTGCAAGTGATCAGCC
TGGAGAGCGGCGACGCTAGCATCCACGACACCGTGGAGAACCTGATCATC
CTGGCCAACAACAGCCTGAGCAGCAACGGCAACGTGACCGAGAGCGGCTG
CAAGGAGTGCGAGGAGCTGGAGGAGAAGAACATCAAGGAGTTCCTGCAGA
GCTTCGTGCACATCGTGCAGATGTTCATCAACACAAGC.

In some embodiments, the second chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 168)
GACATACAGATGACCCAAAGCCCTGCTAGCCTGAGCGCGAGCGTGGGCGA
CAGAGTGACCATCACCTGCCTCGCCTCTCAGACGATTGACACCTGGCTGG
CTTGGTATTTGCAGAAACCTGGGAAAAGCCCTCAGCTGCTGATCTACGCA
GCCACCAATTTAGCCGATGGCGTCCCTTCTAGATTTAGCGGCTCGGGATC
CGGCACCGATTTTAGCTTTACAATTTCATCTCTACAGCCTGAGGACTTCG
CCACCTATTATTGTCAACAAGTATATAGCAGCCCTTTCACCTTCGGCCAA
GGCACCAAGTTAGAGATCAAGGGCGGTGGCGGCTCCGGGGGCGGGGGAAG
TGGAGGTGGGGGGTCACAGATTCAGCTGGTTCAAAGCGGCGGCGAGGTGA
AGAAACCTGGCGCCTCGGTGAGAGTTAGCTGCAAAGCTAGCGGATATTCG
TTTACTGACTACAACGTGTATTGGGTGAGGCAGAGCCCGGGCAAGGGACT
GGAGTGGATTGGGTACATTGACCCCTACAACGGCATAACCATATATGATC
AGAACTTCAAGGGCAAAGCCACGCTGACCGTGGATAAAAGCACAAGCACC
GCTTATATGGAGTTGAGCTCCCTCCGTAGCGAAGACACGGCAGTATACTT
TTGCGCACGGGATGTGACCACGGCCCTAGATTTCTGGGGACAAGGGACAA
CCGTTACTGTTAGTTCCGGCGGTGGGGGCTCGATCACGTGCCCTCCCCCC
ATGTCCGTGGAACACGCAGACATCTGGGTCAAGAGCTACAGCTTGTACTC
CAGGGAGCGGTACATTTGTAACTCTGGTTTCAAGCGTAAAGCCGGCACGT
CCAGCCTGACGGAGTGCGTGTTGAACAAGGCCACGAATGTCGCCCACTGG
ACAACCCCCAGTCTCAAATGCATTAGAGACCCTGCCCTGGTTCACCAAAG
GGATATTCAGATGACGCAGTCGCCAAGCTCACTCTCGGCCTCCGTAGGAG
ACAGAGTTACCATCACCTGCAGAGCTAGCCAAGACATCAGAAACTACCTG
AACTGGTATCAGCAGAAGCCTGGCAAGGCCCCTAAGCTCCTGATCTACTA
CACGAGCAGACTTGAGAGCGGGGTACCATCCCGCTTCAGCGGATCTGGAA
GCGGCACAGACTACACCCTGACCATCTCTAGTCTGCAGCCTGAGGACTTC
GCCACTTACTATTGTCAGCAAGGCAACACCCTGCCTTGGACCTTTGGCCA
AGGCACCAAAGTTGAGATAAAGGGAGGGGGAGGGAGCGGTGGCGGAGGCT
CGGGTGGGGGTGGCTCAGAAGTACAGCTGGTTGAGTCCGGCGGTGGTCTG
GTGCAGCCTGGCGGAAGCCTGAGACTGAGCTGCGCCGCAAGCGGCTATAG
CTTCACTGGCTATACCATGAACTGGGTGCGCCAAGCCCCTGGCAAGGGCC
TGGAATGGGTGGCCCTGATCAACCCTTACAAGGGCGTGAGCACCTACAAT
CAGAAGTTCAAGGACAGATTCACCATCAGCGTGGACAAGAGCAAGAACAC
CGCCTACCTGCAGATGAACAGCCTGAGAGCCGAGGACACTGCGGTCTATT
ACTGTGCTAGAAGCGGCTACTACGGCGACAGCGACTGGTACTTCGACGTG
TGGGGGCAAGGCACCTTGGTAACGGTAAGCTCC.

### IL-7/TRβC/IL-15: Anti-TFscFv/IL-15RαSu/Anti-Mouse CD3scFv

In some embodiments, provided herein is a multi-chain chimeric polypeptide that includes: (a) a first chimeric polypeptide including: (i) a first target-binding domain that includes a human soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TFscFv (e.g., a sequence that is at least 80%, at least 90%, at least 95%, or 100% identical to SEQ ID NO: 131); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-mouse CD3scFv (e.g., a sequence that is at least 80%, at least 90%, at least 95%, or 100% identical to any one of SEQ ID NO: 133).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical to:

(SEQ ID NO: 140)
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EHEDLNKVFPPEVAVFEPSEAEISHTQKATLVCLATGFFPDHVELSWWVN
GKEVHSGVSTDPQPLKEQPALNDSRYCLSSRLRVSATFWQNPRNHFRCQV
QFYGLSENDEWTQDRAKPVTQIVSAEAWGRADNWVNVISDLKKIEDLIQS
MHIDATLYTESDVHPSCKVTAMKCFLLELQVISLESGDASIHDTVENLII
LANNSLSSNGNVTESGCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 174)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSGGGGSGGGGSQIQLVQSGGEVKKPGASVRVSCKASGYS
FTDYNVYWVRQSPGKGLEWIGYIDPYNGITIYDQNFKGKATLTVDKSTST
AYMELSSLRSEDTAVYFCARDVTTALDFWGQGTTVTVSSGGGGSITCPPP
MSVEHADIWVKSYSLYSRERYICNSGFKRKAGTSSLTECVLNKATNVAHW
TTPSLKCIRDPALVHQRDIQMTQSPSSLPASLGDRVTINCQASQDISNYL
NWYQQKPGKAPKLLIYYTNKLADGVPSRFSGSGSGRDSSFTISSLESEDI
GSYYCQQYYNYPWTFGPGTKLEIKGGGGSGGGGSGGGGSEVQLVESGGGL
VQPGKSLKLSCEASGFTFSGYGMHWVRQAPGRGLESVAYITSSSINIKYA
DAVKGRFTVSRDNAKNLLFLQMNILKSEDTAMYYCAREDWDKNYWGQGTM
VTVSS.

In some embodiments, the second chimeric polypeptide further includes a signal sequence at its N-terminal end. In some embodiments the signal peptide sequence is MGWSCIILFLVATATGVHS (SEQ ID NO: 139).

### TGFβRII/TRβC/IL-15: Anti-TFscFv/IL-15RαSu/Anti-CD3scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TFscFv (e.g., a sequence that is at least 80%, at least 90%, at least 95%, or 100% identical to SEQ ID NO: 131); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human CD3scFv (e.g., a sequence that is at least 80%, at least 90%, at least 95%, or 100% identical to SEQ ID NO: 17 or SEQ ID NO: 19).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPDGGGGSIPPHVQKSV
NNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGET
FFMCSCSSDECNDNIIFSEEYNTSNPDEDLNKVFPPEVAVFEPSEAEISH
TQKATLVCLATGFFPDHVELSWWVNGKEVHSGVSTDPQPLKEQPALNDSR
YCLSSRLRVSATFWQNPRNHERCQVQFYGLSENDEWTQDRAKPVTQIVSA
EAWGRADNWVNVISDLKKIEDLIQSMHIDATLYTESDVHPSCKVTAMKCF
LLELQVISLESGDASIHDTVENLIILANNSLSSNGNVTESGCKECEELEE
KNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 166)
DIQMTOSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSGGGGSGGGGSQIQLVQSGGEVKKPGASVRVSCKASGYS
FTDYNVYWVRQSPGKGLEWIGYIDPYNGITIYDQNFKGKATLTVDKSTST
AYMELSSLRSEDTAVYFCARDVTTALDFWGQGTTVTVSSGGGGSITCPPP
MSVEHADIWVKSYSLYSRERYICNSGFKRKAGTSSLTECVLNKATNVAHW
TTPSLKCIRDPALVHQRDIQMTQSPSSLSASVGDRVTITCRASQDIRNYL
NWYQQKPGKAPKLLIYYTSRLESGVPSRFSGSGSGTDYTLTISSLQPEDF
ATYYCQQGNTLPWTFGQGTKVEIKGGGGSGGGGSGGGGSEVQLVESGGGL
VQPGGSLRLSCAASGYSFTGYTMNWVRQAPGKGLEWVALINPYKGVSTYN
QKFKDRFTISVDKSKNTAYLQMNSLRAEDTAVYYCARSGYYGDSDWYFDV
WGQGTLVTVSS.

In some embodiments, the second chimeric polypeptide further includes a signal sequence at its N-terminal end. In some embodiments the signal peptide sequence is MGWSCIILFLVATATGVHS (SEQ ID NO: 139).

In some embodiments, the first chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 169)
ATTCCTCCTCACGTGCAGAAAAGCGTGAACAACGACATGATAGTGACCGA
CAATAACGGCGCGGTCAAGTTCCCACAGCTCTGCAAATTTTGCGACGTGA
GATTTAGTACCTGCGACAATCAGAAATCCTGCATGAGCAATTGTAGCATC
ACTAGCATCTGTGAAAAGCCCCAAGAGGTCTGCGTAGCAGTGTGGAGAAA
AAATGACGAGAACATCACACTAGAGACCGTGTGCCATGACCCTAAGCTGC
CTTATCATGATTTCATCCTTGAAGATGCTGCTAGCCCTAAGTGCATTATG
AAAGAGAAAAAGAAGCCTGGCGAAACCTTTTTCATGTGTAGCTGTAGTAG
CGACGAGTGCAACGATAACATCATCTTCTCGGAGGAGTATAATACATCGA
ATCCTGATGGCGGCGGCGGCAGCATCCCTCCTCATGTTCAGAAGAGCGTG
AATAACGACATGATTGTGACGGATAACAATGGCGCCGTAAAGTTCCCTCA
GCTGTGTAAGTTCTGCGATGTGCGATTTAGCACCTGCGACAATCAGAAGA
GCTGTATGTCCAATTGTTCAATCACAAGCATCTGTGAGAAGCCTCAAGAG
GTGTGCGTGGCCGTTTGGAGAAAGAACGACGAGAACATTACCCTTGAGAC
CGTATGCCACGATCCTAAGCTGCCGTATCACGACTTCATACTGGAGGATG
CCGCTAGCCCTAAATGCATCATGAAAGAAAAAAAAAAGCCTGGTGAGACA
TTTTTTATGTGTAGCTGCTCGTCTGACGAGTGCAATGACAATATCATTTT
CAGCGAAGAATATAACACCTCAAACCCTGACGAGGACCTGAACAAGGTGT
TCCCACCCGAGGTCGCTGTGTTTGAGCCATCAGAAGCAGAGATCTCCCAC
ACCCAAAAGGCCACACTGGTGTGCCTGGCCACAGGCTTCTTCCCCGACCA
CGTGGAGCTGAGCTGGTGGGTGAATGGGAAGGAGGTGCACAGTGGGGTCA
GCACGGACCCGCAGCCCCTCAAGGAGCAGCCCGCCCTCAATGACTCCAGA
TACTGCCTGAGCAGCCGCCTGAGGGTCTCGGCCACCTTCTGGCAGAACCC
CCGCAACCACTTCCGCTGTCAAGTCCAGTTCTACGGGCTCTCGGAGAATG
ACGAGTGGACCCAGGATAGGGCCAAACCCGTCACCCAGATCGTCAGCGCC
GAGGCCTGGGGTAGAGCAGACAACTGGGTGAACGTGATCAGCGACCTGAA
GAAGATCGAGGACCTGATTCAGAGCATGCACATCGACGCCACCCTGTACA
CCGAGAGCGACGTGCACCCTAGCTGCAAGGTGACCGCCATGAAGTGCTTC
CTGCTGGAGCTGCAAGTGATCAGCCTGGAGAGCGGCGACGCTAGCATCCA
CGACACCGTGGAGAACCTGATCATCCTGGCCAACAACAGCCTGAGCAGCA
ACGGCAACGTGACCGAGAGCGGCTGCAAGGAGTGCGAGGAGCTGGAGGAG
AAGAACATCAAGGAGTTCCTGCAGAGCTTCGTGCACATCGTGCAGATGTT
CATCAACACAAGC.

In some embodiments, the second chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 168)
GACATACAGATGACCCAAAGCCCTGCTAGCCTGAGCGCGAGCGTGGGCGA
CAGAGTGACCATCACCTGCCTCGCCTCTCAGACGATTGACACCTGGCTGG
CTTGGTATTTGCAGAAACCTGGGAAAAGCCCTCAGCTGCTGATCTACGCA
GCCACCAATTTAGCCGATGGCGTCCCTTCTAGATTTAGCGGCTCGGGATC
CGGCACCGATTTTAGCTTTACAATTTCATCTCTACAGCCTGAGGACTTCG
CCACCTATTATTGTCAACAAGTATATAGCAGCCCTTTCACCTTCGGCCAA
GGCACCAAGTTAGAGATCAAGGGCGGTGGCGGCTCCGGGGGCGGGGGAAG
TGGAGGTGGGGGGTCACAGATTCAGCTGGTTCAAAGCGGCGGCGAGGTGA
AGAAACCTGGCGCCTCGGTGAGAGTTAGCTGCAAAGCTAGCGGATATTCG
TTTACTGACTACAACGTGTATTGGGTGAGGCAGAGCCCGGGCAAGGGACT
GGAGTGGATTGGGTACATTGACCCCTACAACGGCATAACCATATATGATC
AGAACTTCAAGGGCAAAGCCACGCTGACCGTGGATAAAAGCACAAGCACC
GCTTATATGGAGTTGAGCTCCCTCCGTAGCGAAGACACGGCAGTATACTT
TTGCGCACGGGATGTGACCACGGCCCTAGATTTCTGGGGACAAGGGACAA
CCGTTACTGTTAGTTCCGGCGGTGGGGGCTCGATCACGTGCCCTCCCCCC
ATGTCCGTGGAACACGCAGACATCTGGGTCAAGAGCTACAGCTTGTACTC
CAGGGAGCGGTACATTTGTAACTCTGGTTTCAAGCGTAAAGCCGGCACGT
CCAGCCTGACGGAGTGCGTGTTGAACAAGGCCACGAATGTCGCCCACTGG
ACAACCCCCAGTCTCAAATGCATTAGAGACCCTGCCCTGGTTCACCAAAG
GGATATTCAGATGACGCAGTCGCCAAGCTCACTCTCGGCCTCCGTAGGAG
ACAGAGTTACCATCACCTGCAGAGCTAGCCAAGACATCAGAAACTACCTG
AACTGGTATCAGCAGAAGCCTGGCAAGGCCCCTAAGCTCCTGATCTACTA
CACGAGCAGACTTGAGAGCGGGGTACCATCCCGCTTCAGCGGATCTGGAA
GCGGCACAGACTACACCCTGACCATCTCTAGTCTGCAGCCTGAGGACTTC
GCCACTTACTATTGTCAGCAAGGCAACACCCTGCCTTGGACCTTTGGCCA
AGGCACCAAAGTTGAGATAAAGGGAGGGGGAGGGAGCGGTGGCGGAGGCT
CGGGTGGGGGTGGCTCAGAAGTACAGCTGGTTGAGTCCGGCGGTGGTCTG
GTGCAGCCTGGCGGAAGCCTGAGACTGAGCTGCGCCGCAAGCGGCTATAG
CTTCACTGGCTATACCATGAACTGGGTGCGCCAAGCCCCTGGCAAGGGCC
TGGAATGGGTGGCCCTGATCAACCCTTACAAGGGCGTGAGCACCTACAAT
CAGAAGTTCAAGGACAGATTCACCATCAGCGTGGACAAGAGCAAGAACAC
CGCCTACCTGCAGATGAACAGCCTGAGAGCCGAGGACACTGCGGTCTATT
ACTGTGCTAGAAGCGGCTACTACGGCGACAGCGACTGGTACTTCGACGTG
TGGGGGCAAGGCACCTTGGTAACGGTAAGCTCC.

### TGFβRII/TRβC/IL-15: Anti-TFscFv/IL-1SRαSu/Anti-Mouse CD3scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TFscFv (e.g., a sequence that is at least 80%, at least 90%, at least 95%, or 100% identical to SEQ ID NO: 131); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-mouse CD3scFv (e.g., a sequence that is at least 80%, at least 90%, at least 95%, or 100% identical to any one of SEQ ID NO: 133).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPDGGGGSIPPHVQKSV
NNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGET
FFMCSCSSDECNDNIIFSEEYNTSNPDEDLNKVFPPEVAVFEPSEAEISH
TQKATLVCLATGFFPDHVELSWWVNGKEVHSGVSTDPQPLKEQPALNDSR
YCLSSRLRVSATFWQNPRNHFRCQVQFYGLSENDEWTQDRAKPVTQIVSA
EAWGRADNWVNVISDLKKIEDLIQSMHIDATLYTESDVHPSCKVTAMKCF
LLELQVISLESGDASIHDTVENLIILANNSLSSNGNVTESGCKECEELEE
KNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 174)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSGGGGSGGGGSQIQLVQSGGEVKKPGASVRVSCKASGYS
FTDYNVYWVRQSPGKGLEWIGYIDPYNGITIYDQNFKGKATLTVDKSTST
AYMELSSLRSEDTAVYFCARDVTTALDFWGQGTTVTVSSGGGGSITCPPP
MSVEHADIWVKSYSLYSRERYICNSGFKRKAGTSSLTECVLNKATNVAHW
TTPSLKCIRDPALVHQRDIQMTQSPSSLPASLGDRVTINCQASQDISNYL
NWYQQKPGKAPKLLIYYTNKLADGVPSRFSGSGSGRDSSFTISSLESEDI
GSYYCQQYYNYPWTFGPGTKLEIKGGGGSGGGGSGGGGSEVQLVESGGGL
VQPGKSLKLSCEASGFTFSGYGMHWVRQAPGRGLESVAYITSSSINIKYA
DAVKGRFTVSRDNAKNLLFLQMNILKSEDTAMYYCARFDWDKNYWGQGTM
VTVSS.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-TF(Diabody)/IL-15RαSu/hIL-21

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TF diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an human soluble IL-21 (e.g., a sequence that is at least 80%, at least 90%, at least 95%, or 100% identical to SEQ ID NO: 5).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EHEDLNKVEPPEVAVFEPSEAEISHTQKATLVCLATGFFPDHVELSWWVN
GKEVHSGVSTDPQPLKEQPALNDSRYCLSSRLRVSATFWQNPRNHFRCQV
QFYGLSENDEWTQDRAKPVTQIVSAEAWGRADNWVNVISDLKKIEDLIQS
MHIDATLYTESDVHPSCKVTAMKCFLLELQVISLESGDASIHDTVENLII
LANNSLSSNGNVTESGCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 170)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSQIQLVQSGGEVKKPGASVRVSCKASGYSFTDYNVYWVR
QSPGKGLEWIGYIDPYNGITIYDQNFKGKATLTVDKSTSTAYMELSSLRS
EDTAVYFCARDVTTALDFWGQGTTVTVSSGGGGSGGGGSGGGGSDIQMTQ
SPASLSASVGDRVTITCLASQTIDTWLAWYLQKPGKSPQLLIYAATNLAD
GVPSRESGSGSGTDFSFTISSLQPEDFATYYCQQVYSSPFTFGQGTKLEI
KGGGGSQIQLVQSGGEVKKPGASVRVSCKASGYSFTDYNVYWVRQSPGKG
LEWIGYIDPYNGITIYDQNFKGKATLTVDKSTSTAYMELSSLRSEDTAVY
FCARDVTTALDFWGQGTTVTVSSITCPPPMSVEHADIWVKSYSLYSRERY
ICNSGFKRKAGTSSLTECVLNKATNVAHWTTPSLKCIRDPALVHQRQGQD
RHMIRMRQLIDIVDQLKNYVNDLVPEFLPAPEDVETNCEWSAFSCFQKAQ
ALKSNTGNNERIINVSIKKLKRKPPSTNAGRRQKHRLTCPSCDSYEKKPP
KEFLERFKSLLQKMIHQHLSSRTHGSEDS.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-TF(Diabody)/IL-15RαSu/mIL-21

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TF diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an mouse soluble IL-21 (e.g., a sequence that is at least 80%, at least 90%, at least 95%, or 100% identical to SEQ ID NO: 5 or SEQ ID NO: 124).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EHEDLNKVFPPEVAVFEPSEAEISHTQKATLVCLATGFFPDHVELSWWVN
GKEVHSGVSTDPQPLKEQPALNDSRYCLSSRLRVSATFWQNPRNHFRCQV
QFYGLSENDEWTQDRAKPVTQIVSAEAWGRADNWVNVISDLKKIEDLIQS
MHIDATLYTESDVHPSCKVTAMKCFLLELQVISLESGDASIHDTVENLII
LANNSLSSNGNVTESGCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 171)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSQIQLVQSGGEVKKPGASVRVSCKASGYSFTDYNVYWVR
QSPGKGLEWIGYIDPYNGITIYDQNFKGKATLTVDKSTSTAYMELSSLRS
EDTAVYFCARDVTTALDFWGQGTTVTVSSGGGGSGGGGSGGGGSDIQMTQ
SPASLSASVGDRVTITCLASQTIDTWLAWYLQKPGKSPQLLIYAATNLAD
GVPSRFSGSGSGTDFSFTISSLQPEDFATYYCQQVYSSPFTFGQGTKLEI
KGGGGSQIQLVQSGGEVKKPGASVRVSCKASGYSFTDYNVYWVRQSPGKG
LEWIGYIDPYNGITIYDQNFKGKATLTVDKSTSTAYMELSSLRSEDTAVY
FCARDVTTALDFWGQGTTVTVSSITCPPPMSVEHADIWVKSYSLYSRERY
ICNSGFKRKAGTSSLTECVLNKATNVAHWTTPSLKCIRDPALVHQRQGPD
RLLIRLRHLIDIVEQLKIYENDLDPELLSAPQDVKGHCEHAAFACFQKAK
LKPSNPGNNKTFIIDLVAQLRRRLPARRGGKKQKHIAKCPSCDSYEKRTP
KEFLERLKWLLQKMIHQHLS.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-TFscFv/IL-15RαSu/IL-21

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TFscFv (e.g., a sequence that is at least 80%, at least 90%, at least 95%, or 100% identical to SEQ ID NO: 131); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a soluble IL-21 (e.g., a sequence that is at least 80%, at least 90%, at least 95%, or 100% identical to SEQ ID NO: 5 or SEQ ID NO: 124).

### TGFβRII/TRβC/IL-15: Anti-TFscFv/IL-15RαSu/Anti-Human PD-1

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TFscFv (e.g., a sequence that is at least 80%, at least 90%, at least 95%, or 100% identical to SEQ ID NO: 131); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-human PD-1 antigen-binding domain (e.g., an anti-human PD-1 diabody or an anti-human PD-1 scFv). In some embodiments, the anti-human PD-1 antigen-binding domain is an anti-human PD-1 diabody (e.g., a sequence that is at least 80%, at least 90%, at least 95%, or 100% identical to SEQ ID NO: 21). In some embodiments, the anti-human PD-1 antigen-binding domain is an anti-human PD-1 scFv (e.g., a sequence that is at least 80%, at least 90%, at least 95%, or 100% identical to SEQ ID NO: 135).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPDGGGGSIPPHVQKSV
NNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGET
FFMCSCSSDECNDNIIFSEEYNTSNPDEDLNKVFPPEVAVFEPSEAEISH
TQKATLVCLATGFFPDHVELSWWVNGKEVHSGVSTDPQPLKEQPALNDSR
YCLSSRLRVSATFWQNPRNHERCQVQFYGLSENDEWTQDRAKPVTQIVSA
EAWGRADNWVNVISDLKKIEDLIQSMHIDATLYTESDVHPSCKVTAMKCF
LLELQVISLESGDASIHDTVENLIILANNSLSSNGNVTESGCKECEELEE
KNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 172)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSGGGGSGGGGSQIQLVQSGGEVKKPGASVRVSCKASGYS
FTDYNVYWVRQSPGKGLEWIGYIDPYNGITIYDQNFKGKATLTVDKSTST
AYMELSSLRSEDTAVYFCARDVTTALDFWGQGTTVTVSSGGGGSITCPPP
MSVEHADIWVKSYSLYSRERYICNSGFKRKAGTSSLTECVLNKATNVAHW
TTPSLKCIRGGGGSEIVLTQSPATLSLSPGERATLSCRASKGVSTSGYSY
LHWYQQKPGQAPRLLIYLASYLESGVPARFSGSGSGTDFTLTISSLEPED
FAVYYCQHSRDLPLTFGGGTKVEIKGGGGSGGGGSGGGGSQVQLVQSGVE
VKKPGASVKVSCKASGYTFTNYYMYWVRQAPGQGLEWMGGINPSNGGTNF
NEKFKNRVTLTTDSSTTTAYMELKSLQFDDTAVYYCARRDYRFDMGFDYW
GQGTTVTVSS.

In some embodiments, the second chimeric polypeptide further includes a signal sequence at its N-terminal end. In some embodiments the signal peptide sequence is MGWSCIILFLVATATGVHS (SEQ ID NO: 139).
TGFβRII/TRβC/IL-15: Anti-TFscFv/IL-15RαSu/Anti-mPD-1 scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TFscFv (e.g., a sequence that is at least 80%, at least 90%, at least 95%, or 100% identical to SEQ ID NO: 131); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-mouse PD-1 antigen-binding domain (e.g., an anti-mouse PD-1 diabody or an anti-mouse PD-1 scFv). In some embodiments, the anti-mouse PD-1 antigen-binding domain is an anti-mouse PD-1 diabody (e.g., a sequence that is at least 80%, at least 90%, at least 95%, or 100% identical to SEQ ID NO: 134). In some embodiments, the anti-mouse PD-1 antigen-binding domain is an anti-mouse PD-1 scFv (e.g., a sequence that is at least 80%, at least 90%, at least 95%, or 100% identical to SEQ ID NO: 136).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPDGGGGSIPPHVQKSV
NNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGET
FFMCSCSSDECNDNIIFSEEYNTSNPDEDLNKVFPPEVAVFEPSEAEISH
TQKATLVCLATGFFPDHVELSWWVNGKEVHSGVSTDPQPLKEQPALNDSR
YCLSSRLRVSATFWQNPRNHFRCQVQFYGLSENDEWTQDRAKPVTQIVSA
EAWGRADNWVNVISDLKKIEDLIQSMHIDATLYTESDVHPSCKVTAMKCF
LLELQVISLESGDASIHDTVENLIILANNSLSSNGNVTESGCKECEELEE
KNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 176)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSGGGGSGGGGSQIQLVQSGGEVKKPGASVRVSCKASGYS
FTDYNVYWVRQSPGKGLEWIGYIDPYNGITIYDQNFKGKATLTVDKSTST
AYMELSSLRSEDTAVYFCARDVTTALDFWGQGTTVTVSSGGGGSITCPPP
MSVEHADIWVKSYSLYSRERYICNSGFKRKAGTSSLTECVLNKATNVAHW
TTPSLKCIRGGGGSRTDVALTQTPVAQPVTLGDQASISCRSSQSLVHSNG
RTYLEWYLQKPGQSPQLLIYKVSNRFSGVPDRFIGSGSGSDFTLTISRVE
PEDLGVYYCFQATHDPNTFGAGTKLELKGGGGSGGGGSGGGGSQVQLQQS
GAELVKPGSSVKISCKASGYTFTSHFIHWIKQQPGNGLEWIGGIYPGDGD
TEYNQQFNGKATLTADKSSSTAYMRLSSLTSEDSAVYFCATRVPSYWFFD
FWGPGTMVTVSS.

In some embodiments, the second chimeric polypeptide further includes a signal sequence at its N-terminal end. In some embodiments the signal peptide sequence is MGWSCIILFLVATATGVHS (SEQ ID NO: 139).

### TGFβRII/TRβC/IL-15: Anti-TF(Diabody)/IL-15RαSu/Anti-PD-1

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-PD-1 antigen-binding domain (e.g., an anti-PD-1 diabody or an anti-PD-1 scFv). In some embodiments, the anti-PD-1 antigen-binding domain is an anti-PD-1 diabody (e.g., a sequence that is at least 80%, at least 90%, at least 95%, or 100% identical to SEQ ID NO: 21 or SEQ ID NO: 134). In some embodiments, the anti-PD-1 antigen-binding domain is an anti-PD-1 scFv (e.g., a sequence that is at least 80%, at least 90%, at least 95%, or 100% identical to SEQ ID NO: 135 or SEQ ID NO: 136).

### IL-7/TRβC/IL-15: Anti-TFscFv/IL-15RαSu/Anti-PD-1

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TFscFv (e.g., a sequence that is at least 80%, at least 90%, at least 95%, or 100% identical to SEQ ID NO: 131); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-PD-1 antigen-binding domain. In some embodiments, the anti-PD-1 antigen-binding domain is an anti-PD-1 diabody (e.g., a sequence that is at least 80%, at least 90%, at least 95%, or 100% identical to SEQ ID NO: 21 or SEQ ID NO: 134). In some embodiments, the anti-PD-1 antigen-binding domain is an anti-PD-1 scFv (e.g., a sequence that is at least 80%, at least 90%, at least 95%, or 100% identical to SEQ ID NO: 135 or SEQ ID NO: 136).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EHEDLNKVFPPEVAVFEPSEAEISHTQKATLVCLATGFFPDHVELSWWVN
GKEVHSGVSTDPQPLKEQPALNDSRYCLSSRLRVSATFWQNPRNHFRCQV
QFYGLSENDEWTQDRAKPVTQIVSAEAWGRADNWVNVISDLKKIEDLIQS
MHIDATLYTESDVHPSCKVTAMKCFLLELQVISLESGDASIHDTVENLII
LANNSLSSNGNVTESGCKECEELEEKNIKEFLQSFVHIVQMFINTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 172)
DIQMTQSPASLSASVGDRVTITCLASQTIDTWLAWYLQKPGKSPQLLIYA
ATNLADGVPSRFSGSGSGTDESFTISSLQPEDFATYYCQQVYSSPFTFGQ
GTKLEIKGGGGSGGGGSGGGGSQIQLVQSGGEVKKPGASVRVSCKASGYS
FTDYNVYWVRQSPGKGLEWIGYIDPYNGITIYDQNFKGKATLTVDKSTST
AYMELSSLRSEDTAVYFCARDVTTALDFWGQGTTVTVSSGGGGSITCPPP
MSVEHADIWVKSYSLYSRERYICNSGFKRKAGTSSLTECVLNKATNVAHW
TTPSLKCIRGGGGSEIVLTQSPATLSLSPGERATLSCRASKGVSTSGYSY
LHWYQQKPGQAPRLLIYLASYLESGVPARFSGSGSGTDFTLTISSLEPED
FAVYYCQHSRDLPLTFGGGTKVEIKGGGGSGGGGSGGGGSQVQLVQSGVE
VKKPGASVKVSCKASGYTFTNYYMYWVRQAPGQGLEWMGGINPSNGGTNF
NEKFKNRVTLTTDSSTTTAYMELKSLQFDDTAVYYCARRDYRFDMGFDYW
GQGTTVTVSS.

In some embodiments, the second chimeric polypeptide further includes a signal sequence at its N-terminal end. In some embodiments the signal peptide sequence is MGWSCIILFLVATATGVHS (SEQ ID NO: 139).

### IL-7/TRβC/IL-15: Anti-TF(Diabody)/IL-15RαSu/Anti-PD-1

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TF diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-PD-1 antigen-binding domain. In some embodiments, the anti-PD-1 antigen-binding domain is an anti-PD-1 diabody (e.g., a sequence that is at least 80%, at least 90%, at least 95%, or 100% identical to SEQ ID NO: 21 or SEQ ID NO: 134). In some embodiments, the anti-PD-1 antigen-binding domain is an anti-PD-1 scFv (e.g., a sequence that is at least 80%, at least 90%, at least 95%, or 100% identical to SEQ ID NO: 135 or SEQ ID NO: 136).

TGFβRII/TRβC/IL-15: Anti-TFscFv/IL-15RαSu/Anti-CD16a scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TFscFv (e.g., a sequence that is at least 80%, at least 90%, at least 95%, or 100% identical to SEQ ID NO: 131); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-CD16a scFv (e.g., a sequence that is at least 80%, at least 90%, at least 95%, or 100% identical to SEQ ID NO: 119).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPDGGGGSIPPHVQKSV
NNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGET
FFMCSCSSDECNDNIIFSEEYNTSNPDEDLNKVFPPEVAVFEPSEAEISH
TQKATLVCLATGFFPDHVELSWWVNGKEVHSGVSTDPQPLKEQPALNDSR
YCLSSRLRVSATFWQNPRNHFRCQVQFYGLSENDEWTQDRAKPVTQIVSA
EAWGRADNWVNVISDLKKIEDLIQSMHIDATLYTESDVHPSCKVTAMKCF
LLELQVISLESGDASIHDTVENLIILANNSLSSNGNVTESGCKECEELEE
KNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 173)
DIQMTQSPASLSASVGDRVTITCLASQTIDTWLAWYLQKPGKSPQLLIYA
ATNLADGVPSRFSGSGSGTDFSFTISSLQPEDFATYYCQQVYSSPFTFGQ
GTKLEIKGGGGSGGGGSGGGGSQIQLVQSGGEVKKPGASVRVSCKASGYS
FTDYNVYWVRQSPGKGLEWIGYIDPYNGITIYDQNFKGKATLTVDKSTST
AYMELSSLRSEDTAVYFCARDVTTALDFWGQGTTVTVSSGGGGSITCPPP
MSVEHADIWVKSYSLYSRERYICNSGFKRKAGTSSLTECVLNKATNVAHW
TTPSLKCIRDPALVHQRSELTQDPAVSVALGQTVRITCQGDSLRSYYASW
YQQKPGQAPVLVIYGKNNRPSGIPDRFSGSSSGNTASLTITGAQAEDEAD
YYCNSRDSSGNHVVFGGGTKLTVGHGGGGSGGGGSGGGGSEVQLVESGGG
VVRPGGSLRLSCAASGFTFDDYGMSWVRQAPGKGLEWVSGINWNGGSTGY
ADSVKGRFTISRDNAKNSLYLQMNSLRAEDTAVYYCARGRSLLFDYWGQG
TLVTVSR.

In some embodiments, the second chimeric polypeptide further includes a signal sequence at its N-terminal end. In some embodiments the signal peptide sequence is MGWSCIILFLVATATGVHS (SEQ ID NO: 139).
TGFβRII/TRβC/IL-15: Anti-TF(Diabody)/IL-1SRαSu/Anti-CD16a scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human TF diabody (e.g., a sequence that is at least 80%, at least 90%, at least 95%, or 100% identical to SEQ ID NO: 15); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-CD16a scFv (e.g., a sequence that is at least 80%, at least 90%, at least 95%, or 100% identical to SEQ ID NO: 119).

### Exemplary Multi-Chain Chimeric Polypeptides—Type F

In some embodiments of any of the multi-chain chimeric polypeptides described herein, the first target-binding domain and the second targeting-binding domain each independently bind specifically to an IL-7 receptor, to PD-1, or to an IL-4 receptor. In some embodiments of these multi-chain chimeric polypeptides, the first target-binding domain and the second target-binding domain both bind specifically to an IL-7 receptor, to PD-1, or to an IL-4 receptor.

In some embodiments of these multi-chain chimeric polypeptides, the first target-binding domain binds specifically to an IL-7 receptor, and the second target-binding domain binds specifically to an IL-4 receptor. In some embodiments of these multi-chain chimeric polypeptides, the first target-binding domain binds specifically to an IL-4 receptor, and the second target-binding domain binds specifically to an IL-7 receptor. In some embodiments of these multi-chain chimeric polypeptides, the first target-binding domain binds specifically to an IL-7 receptor, and the second target-binding domain binds specifically to PD-1. In some embodiments of these multi-chain chimeric polypeptides, the first target-binding domain binds specifically to PD-1, and the second target-binding domain bind specifically to an IL-7 receptor.

In some examples of these multi-chain chimeric polypeptides, the first target-binding domain and the soluble TRβC domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble TRβC domain in the first chimeric polypeptide.

In some embodiments of these multi-chain chimeric polypeptides, the soluble TRβC domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble TRβC domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble T cell receptor β-chain constant region (TRβC) domain can be any of the exemplary soluble T cell receptor β-chain constant region (TRβC) domains described herein.

In some embodiments of these multi-chain chimeric polypeptides, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these multi-chain chimeric polypeptides, the pair of affinity domains can be any of the exemplary pairs of affinity domains described herein. In some embodiments, the pair of affinity domains is a sushi domain from an alpha chain of human IL-15 receptor (IL-15Rα) and a soluble TL-15. In some embodiments, the second domain of the pair of affinity domains is a sushi domain from an alpha chain of human IL-15 receptor (IL-15Rα) and the first domain of the pair of affinity domains a soluble IL-15. In some embodiments, the first domain of the pair of affinity domains is a sushi domain from an alpha chain of human IL-15 receptor (IL-15Rα) and the second domain of the pair of affinity domains a soluble IL-15.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 11.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 115.

In some embodiments, a soluble IL-15 can include a sequence that is at least 70% identical, at least 75% identical, at least 80% identical, at least 85% identical, at least 90% identical, at least 95% identical, at least 99% identical, or 100% identical to SEQ ID NO: 13.

In some embodiments of these multi-chain chimeric polypeptides, one or both of the first target-binding domain and the second target-binding domain is a soluble IL-7 (e.g., a soluble human IL-7 polypeptide) or a soluble IL-4 (e.g., a soluble IL-4 polypeptide). In some embodiments of these multi-chain chimeric polypeptides, the first target-binding domain and the second target-binding domain are each independently a soluble IL-7 or a soluble IL-4. In some embodiments of these multi-chain chimeric polypeptides, the first target-binding domain is a soluble IL-7 and the second target-binding domain is a soluble IL-4. In some embodiments of these multi-chain chimeric polypeptides, the first target-binding domain is a soluble IL-7 and the second target-binding domain is a soluble human IL-4. In some embodiments of these multi-chain chimeric polypeptides, the first target-binding domain is a soluble IL-7 and the second target-binding domain is a soluble mouse IL-4.

In some embodiments of these multi-chain chimeric polypeptides, one or both of the first target-binding domain and the second target-binding domain is a soluble IL-7 (e.g., a soluble human IL-7 polypeptide) or an anti-PD-1 antigen-binding domain. In some embodiments of these multi-chain chimeric polypeptides, the first target-binding domain and the second target-binding domain are each independently a soluble IL-7 or an anti-PD-1 antigen-binding domain. In some embodiments of these multi-chain chimeric polypeptides, the first target-binding domain is a soluble IL-7 and the second target-binding domain is an anti-PD-1 antigen-binding domain. In some embodiments of these multi-chain chimeric polypeptides, the first target-binding domain is a soluble IL-7 and the second target-binding domain is an anti-PD-1 antigen-binding domain.

A non-limiting example of an IL-7 that binds specifically to an IL-7 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 4.

A non-limiting example of an IL-4 that binds specifically to an IL-4 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 113.

In some embodiments, a mouse soluble IL-4 can include a sequence that is at least 70% identical, at least 75% identical, at least 80% identical, at least 85% identical, at least 90% identical, at least 95% identical, at least 99% identical, or 100% identical to SEQ ID NO:128.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 64.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 21.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 22.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 134.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 135.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 136.

In some embodiments, the second chimeric polypeptide further comprises one or more additional target-binding domains at the N-terminal end or the C-terminal end of the second chimeric polypeptide. In some embodiments, the second chimeric polypeptide further comprises one or more additional target-binding domains at the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

In some embodiments, the additional target-binding domain includes or is a soluble IL-21. A non-limiting example of an IL-21 that binds specifically to an IL-21 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 5.

In some embodiments, an IL-21 that binds specifically to an IL-21 receptor can be encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 6 or 23.

In some embodiments, an IL-21 that binds specifically to an IL-21 receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 124.

In some embodiments, the additional target-binding domain includes or is a soluble human IL-21. In some embodiments, the additional target-binding domain includes or is a soluble mouse IL-21.

IL-7/TRβC/IL-15: hIL-4/IL-15RαSu/IL-21

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a human IL-4 (e.g., a sequence that is at least 80%, at least 90%, at least 95%, or 100% identical to SEQ ID NO: 113); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a soluble IL-21 (e.g., a sequence that is at least 80%, at least 90%, at least 95%, or 100% identical to SEQ ID NO: 5 or SEQ ID NO: 124).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EHEDLNKVFPPEVAVFEPSEAEISHTQKATLVCLATGFFPDHVELSWWVN
GKEVHSGVSTDPQPLKEQPALNDSRYCLSSRLRVSATFWQNPRNHFRCQV
QFYGLSENDEWTQDRAKPVTQIVSAEAWGRADNWVNVISDLKKIEDLIQS
MHIDATLYTESDVHPSCKVTAMKCFLLELQVISLESGDASIHDTVENLII
LANNSLSSNGNVTESGCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 177)
MGLTSQLLPPLFFLLACAGNFVHGHKCDITLQEIIKTLNSLTEQKTLCTE
LTVTDIFAASKNTTEKETFCRAATVLRQFYSHHEKDTRCLGATAQQFHRH
KQLIRFLKRLDRNLWGLAGLNSCPVKEANQSTLENFLERLKTIMREKYSK
CSSITCPPPMSVEHADIWVKSYSLYSRERYICNSGFKRKAGTSSLTECVL
NKATNVAHWTTPSLKCIRQGQDRHMIRMRQLIDIVDQLKNYVNDLVPEFL
PAPEDVETNCEWSAFSCFQKAQLKSANTGNNERIINVSIKKLKRKPPSTN
AGRRQKHRLTCPSCDSYEKKPPKEFLERFKSLLQKMIHQHLSSRTHGSED
S.

In some embodiments, the second chimeric polypeptide further includes a signal sequence at its N-terminal end.
IL-7/TRβC/IL-15: mIL-4/IL-15RαSu/IL-21

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a mouse IL-4 (e.g., a sequence that is at least 80%, at least 90%, at least 95%, or 100% identical to SEQ ID NO: 34); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a soluble IL-21 (e.g., a sequence that is at least 80%, at least 90%, at least 95%, or 100% identical to SEQ ID NO: 5 or SEQ ID NO: 124).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EHEDLNKVEPPEVAVFEPSEAEISHTQKATLVCLATGFFPDHVELSWWVN
GKEVHSGVSTDPQPLKEQPALNDSRYCLSSRLRVSATFWQNPRNHFRCQV
QFYGLSENDEWTQDRAKPVTQIVSAEAWGRADNWVNVISDLKKIEDLIQS
MHIDATLYTESDVHPSCKVTAMKCFLLELQVISLESGDASIHDTVENLII
LANNSLSSNGNVTESGCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 178)
MGLNPQLVVILLFFLECTRSHIHGCDKNHLREIIGILNEVTGEGTPCTEMDVPN
VLTATKNTTESELVCRASKVLRIFYLKHGKTPCLKKNSSVLMELQRLFRAFRCL
DSSISCTMNESKSTSLKDFLESLKSIMQMDYSITCPPPMSVEHADIWVKSYSLY
SRERYICNSGFKRKAGTSSLTECVLNKATNVAHWTTPSLKCIRHKSSPQGPDRL
LIRLRHLIDIVEQLKIYENDLDPELLSAPQDVKGHCEHAAFACFQKAKLKPSNP
GNNKTFIIDLVAQLRRRLPARRGGKKQKHIAKCPSCDSYEKRTPKEFLERLKWL
LQKMIHQHLS.

In some embodiments, the second chimeric polypeptide further includes a signal sequence at its N-terminal end.

### IL-7/TRβC/IL-15: Anti-PD-1-scFv/IL-15RαSu/IL-21

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 scFv (e.g., a sequence that is at least 80%, at least 90%, at least 95%, or 100% identical to SEQ ID NO: 135 or SEQ ID NO: 136); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a soluble IL-21 (e.g., a sequence that is at least 80%, at least 90%, at least 95%, or 100% identical to SEQ ID NO: 5 or SEQ ID NO: 124).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDANKEGM
FLFRAARKLRQFLKMNSTGDFDLHLLKVSEGTTILLNCTGQVKGRKPAALGEAQ
PTKSLEENKSLKEQKKLNDLCFLKRLLQEIKTCWNKILMGTKEHEDLNKVEPPE
VAVFEPSEAEISHTQKATLVCLATGFFPDHVELSWWVNGKEVHSGVSTDPQPLK
EQPALNDSRYCLSSRLRVSATFWQNPRNHFRCQVQFYGLSENDEWTQDRAKPVT
QIVSAEAWGRADNWVNVISDLKKIEDLIQSMHIDATLYTESDVHPSCKVTAMKC
FLLELQVISLESGDASIHDTVENLIILANNSLSSNGNVTESGCKECEELEEKNI
KEFLQSFVHIVQMFINTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 179)
EIVLTQSPATLSLSPGERATLSCRASKGVSTSGYSYLHWYQQKPGQAPRLLIYL
ASYLESGVPARFSGSGSGTDFTLTISSLEPEDFAVYYCQHSRDLPLTFGGGTKV
EIKGGGGSGGGGSGGGGSQVQLVQSGVEVKKPGASVKVSCKASGYTFTNYYMYW
VRQAPGQGLEWMGGINPSNGGTNFNEKFKNRVTLTTDSSTTTAYMELKSLQFDD
TAVYYCARRDYRFDMGFDYWGQGTTVTVSSGGGGSITCPPPMSVEHADIWVKSY
SLYSRERYICNSGFKRKAGTSSLTECVLNKATNVAHWTTPSLKCIRQGQDRHMI
RMRQLIDIVDQLKNYVNDLVPEFLPAPEDVETNCEWSAFSCFQKAQLKSANTGN
NERIINVSIKKLKRKPPSTNAGRRQKHRLTCPSCDSYEKKPPKEFLERFKSLLQ
KMIHQHLSSRTHGSEDS.

In some embodiments, the second chimeric polypeptide further includes a signal sequence at its N-terminal end. In some embodiments the signal peptide sequence is MGVKVLFALICIAVAEA (SEQ ID NO: 137).

### IL-7/TRβC/IL-15: Anti-PD-1(Diabody)/IL-1SRαSu/IL-21

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 diabody (e.g., a sequence that is at least 80%, at least 90%, at least 95%, or 100% identical to SEQ ID NO: 21 or SEQ ID NO: 134); (ii) a second domain of a pair of affinity domains that includes the sushi domain from an alpha chain of TL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a soluble IL-21 (e.g., a sequence that is at least 80%, at least 90%, at least 95%, or 100% identical to SEQ ID NO: 5 or SEQ ID NO: 124).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDANKEGM
FLFRAARKLRQFLKMNSTGDFDLHLLKVSEGTTILLNCTGQVKGRKPAALGEAQ
PTKSLEENKSLKEQKKLNDLCFLKRLLQEIKTCWNKILMGTKEHEDLNKVFPPE
VAVFEPSEAEISHTQKATLVCLATGFFPDHVELSWWVNGKEVHSGVSTDPQPLK
EQPALNDSRYCLSSRLRVSATFWQNPRNHFRCQVQFYGLSENDEWTQDRAKPVT
QIVSAEAWGRADNWVNVISDLKKIEDLIQSMHIDATLYTESDVHPSCKVTAMKC
FLLELQVISLESGDASIHDTVENLIILANNSLSSNGNVTESGCKECEELEEKNI
KEFLQSFVHIVQMFINTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 180)
QVQLVQSGVEVKKPGASVKVSCKASGYTFTNYYMYWVRQAPGQGLEWMGGINPS
NGGTNFNEKFKNRVTLTTDSSTTTAYMELKSLQFDDTAVYYCARRDYRFDMGFD
YWGQGTTVTVSSGGGGSEIVLTQSPATLSLSPGERATLSCRASKGVSTSGYSYL
HWYQQKPGQAPRLLIYLASYLESGVPARFSGSGSGTDFTLTISSLEPEDFAVYY
CQHSRDLPLTFGGGTKVEIKGGGGSGGGGSGGGGSQVQLVQSGVEVKKPGASVK
VSCKASGYTFTNYYMYWVRQAPGQGLEWMGGINPSNGGTNFNEKFKNRVTLTTD
SSTTTAYMELKSLQFDDTAVYYCARRDYRFDMGFDYWGQGTTVTVSSGGGGSEI
VLTQSPATLSLSPGERATLSCRASKGVSTSGYSYLHWYQQKPGQAPRLLIYLAS
YLESGVPARFSGSGSGTDFTLTISSLEPEDFAVYYCQHSRDLPLTFGGGTKVEI
KITCPPPMSVEHADIWVKSYSLYSRERYICNSGFKRKAGTSSLTECVLNKATNV
AHWTTPSLKCIRQGQDRHMIRMRQLIDIVDQLKNYVNDLVPEFLPAPEDVETNC
EWSAFSCFQKAQLKSANTGNNERIINVSIKKLKRKPPSTNAGRRQKHRLTCPSC
DSYEKKPPKEFLERFKSLLQKMIHQHLSSRTHGSEDS.

In some embodiments, the second chimeric polypeptide further includes a signal sequence at its N-terminal end. In some embodiments the signal peptide sequence is MGVKVLFALICIAVAEA (SEQ ID NO: 137).

### IL-7/TRβC/IL-15: Anti-PD-1-scFv/IL-15RαSu

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 scFv (e.g., a sequence that is at least 80%, at least 90%, at least 95%, or 100% identical to SEQ ID NO: 135 or SEQ ID NO: 136); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDANKEGM
FLFRAARKLRQFLKMNSTGDFDLHLLKVSEGTTILLNCTGQVKGRKPAALGEAQ
PTKSLEENKSLKEQKKLNDLCFLKRLLQEIKTCWNKILMGTKEHEDLNKVFPPE
VAVFEPSEAEISHTQKATLVCLATGFFPDHVELSWWVNGKEVHSGVSTDPQPLK
EQPALNDSRYCLSSRLRVSATFWQNPRNHERCQVQFYGLSENDEWTQDRAKPVT
QIVSAEAWGRADNWVNVISDLKKIEDLIQMHIDATLYTESDVHPSCKVTAMKC
FLLELQVISLESGDASIHDTVENLIILANNSLSSNGNVTESGCKECEELEEKNI
KEFLQSFVHIVQMFINTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 181)
EIVLTQSPATLSLSPGERATLSCRASKGVSTSGYSYLHWYQQKPGQAPRLLIYL
ASYLESGVPARFSGSGSGTDFTLTISSLEPEDFAVYYCQHSRDLPLTFGGGTKV
EIKGGGGSGGGGSGGGGSQVQLVQSGVEVKKPGASVKVSCKASGYTFTNYYMYW
VRQAPGQGLEWMGGINPSNGGTNFNEKFKNRVTLTTDSSTTTAYMELKSLQFDD
TAVYYCARRDYRFDMGFDYWGQGTTVTVSSGGGGSITCPPPMSVEHADIWVKSY
SLYSRERYICNSGFKRKAGTSSLTECVLNKATNVAHWTTPSLKCIR.

In some embodiments, the second chimeric polypeptide further includes a signal sequence at its N-terminal end. In some embodiments the signal peptide sequence is MGVKVLFALICIAVAEA (SEQ ID NO: 137).

### IL-7/TRβC/IL-15: Anti-PD-1(Diabody)/IL-1SRαSu

In some embodiments, provided herein is a multi-chain chimeric polypeptide that includes: (a) a first chimeric polypeptide including: (i) a first target-binding domain that includes a soluble IL-7 (e.g., a sequence that is at least 80%, at least 90%, at least 95%, or 100% identical to SEQ ID NO: 4); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 diabody (e.g., a sequence that is at least 80%, at least 90%, at least 95%, or 100% identical to SEQ ID NO: 21 or SEQ ID NO: 134); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 140)
DCDIEGKDGKQYESVLMVSIDQLLDSMKEIGSNCLNNEFNFFKRHICDANKEGM
FLFRAARKLRQFLKMNSTGDFDLHLLKVSEGTTILLNCTGQVKGRKPAALGEAQ
PTKSLEENKSLKEQKKLNDLCFLKRLLQEIKTCWNKILMGTKEHEDLNKVFPPE
VAVFEPSEAEISHTQKATLVCLATGFFPDHVELSWWVNGKEVHSGVSTDPQPLK
EQPALNDSRYCLSSRLRVSATFWQNPRNHERCQVQFYGLSENDEWTQDRAKPVT
QIVSAEAWGRADNWVNVISDLKKIEDLIQSMHIDATLYTESDVHPSCKVTAMKC
FLLELQVISLESGDASIHDTVENLIILANNSLSSNGNVTESGCKECEELEEKNI
KEFLQSFVHIVQMFINTS.

In some embodiments, the first chimeric polypeptide further includes a signal sequence at its N-terminal end.

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 182)
QVQLVQSGVEVKKPGASVKVSCKASGYTFTNYYMYWVRQAPGQGLEWMGGINPS
NGGTNFNEKFKNRVTLTTDSSTTTAYMELKSLQFDDTAVYYCARRDYRFDMGED
YWGQGTTVTVSSGGGGSEIVLTQSPATLSLSPGERATLSCRASKGVSTSGYSYL
HWYQQKPGQAPRLLIYLASYLESGVPARFSGSGSGTDFTLTISSLEPEDFAVYY
CQHSRDLPLTFGGGTKVEIKGGGGSGGGGSGGGGSQVQLVQSGVEVKKPGASVK
VSCKASGYTFTNYYMYWVRQAPGQGLEWMGGINPSNGGTNFNEKFKNRVTLTTD
SSTTTAYMELKSLQFDDTAVYYCARRDYRFDMGFDYWGQGTTVTVSSGGGGSEI
VLTQSPATLSLSPGERATLSCRASKGVSTSGYSYLHWYQQKPGQAPRLLIYLAS
YLESGVPARFSGSGSGTDFTLTISSLEPEDFAVYYCQHSRDLPLTFGGGTKVEI
KITCPPPMSVEHADIWVKSYSLYSRERYICNSGFKRKAGTSSLTECVLNKATNV
AHWTTPSLKCIR.

In some embodiments, the second chimeric polypeptide further includes a signal sequence at its N-terminal end. In some embodiments the signal peptide sequence is MGVKVLFALICIAVAEA (SEQ ID NO: 137).

### Exemplary Multi-Chain Chimeric Polypeptides—Type G

In some embodiments of any of the multi-chain chimeric polypeptides described herein, the first target-binding domain and the second targeting-binding domain each independently bind specifically to PD-1, to a ligand of TGF-βRII, or to tissue factor.

In some embodiments of these multi-chain chimeric polypeptides, the first target-binding domain and the second target-binding domain both bind specifically to PD-1 or to tissue factor. In some embodiments of these multi-chain chimeric polypeptides, the first target-binding domain binds specifically to PD-1, and the second target-binding domain binds specifically to tissue factor. In some embodiments of these multi-chain chimeric polypeptides, the first target-binding domain binds specifically to tissue factor, and the second target-binding domain binds specifically to a PD-1.

In some embodiments of these multi-chain chimeric polypeptides, the first target-binding domain and the second target-binding domain both bind specifically to a ligand of TGF-βRII or to PD-1. In some embodiments of these multi-chain chimeric polypeptides, the first target-binding domain binds specifically to a ligand of TGF-βRII, and the second target-binding domain binds specifically to PD-1. In some embodiments of these multi-chain chimeric polypeptides, the first target-binding domain binds specifically to PD-1, and the second target-binding domain bind specifically to a ligand of TGF-βRII.

In some examples of these multi-chain chimeric polypeptides, the first target-binding domain and the soluble TRβC domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble TRβC domain in the first chimeric polypeptide.

In some embodiments of these multi-chain chimeric polypeptides, the soluble TRβC domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble TRβC domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble T cell receptor β-chain constant region (TRβC) domain can be any of the exemplary soluble T cell receptor β-chain constant region (TRβC) domains described herein.

In some embodiments of these multi-chain chimeric polypeptides, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these multi-chain chimeric polypeptides, the pair of affinity domains can be any of the exemplary pairs of affinity domains described herein. In some embodiments, the pair of affinity domains is a sushi domain from an alpha chain of human IL-15 receptor (IL-15Rα) and a soluble TL-15. In some embodiments, the second domain of the pair of affinity domains is a sushi domain from an alpha chain of human IL-15 receptor (IL-15Rα) and the first domain of the pair of affinity domains a soluble IL-15. In some embodiments, the first domain of the pair of affinity domains is a sushi domain from an alpha chain of human IL-15 receptor (IL-15Rα) and the second domain of the pair of affinity domains a soluble IL-15.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 11.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 115.

In some embodiments, a soluble IL-15 can include a sequence that is at least 70% identical, at least 75% identical, at least 80% identical, at least 85% identical, at least 90% identical, at least 95% identical, at least 99% identical, or 100% identical to SEQ ID NO: 13.

In some embodiments of these multi-chain chimeric polypeptides, one or both of the first target-binding domain and the second target-binding domain is an anti-PD-1 antigen-binding domain or an anti-tissue factor antigen-binding domain. In some embodiments of these multi-chain chimeric polypeptides, the first target-binding domain and the second target-binding domain are each independently an anti-PD-1 antigen-binding domain or an anti-tissue factor antigen-binding domain. In some embodiments of these multi-chain chimeric polypeptides, the first target-binding domain is an anti-PD-1 antigen-binding domain, and the second target-binding domain is an anti-tissue factor antigen-binding domain. In some embodiments of these multi-chain chimeric polypeptides, the first target-binding domain is an anti-tissue factor antigen-binding domain, and the second target-binding domain is an anti-PD-1 antigen-binding domain.

In some embodiments of these multi-chain chimeric polypeptides, one or both of the first target-binding domain and the second target-binding domain is a soluble TGFβRII receptor or an anti-PD-1 antigen-binding domain. In some embodiments of these multi-chain chimeric polypeptides, the first target-binding domain and the second target-binding domain are each independently a soluble TGFβRII receptor or an anti-PD-1 antigen-binding domain. In some embodiments of these multi-chain chimeric polypeptides, the first target-binding domain is a soluble TGFβRII receptor, and the second target-binding domain is an anti-PD-1 antigen-binding domain. In some embodiments of these multi-chain chimeric polypeptides, the first target-binding domain is an anti-PD-1 antigen-binding domain, and the second target-binding domain is a soluble TGFβRII receptor.

In some embodiments, an anti-PD-1 antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64.

In some embodiments, an anti-PD-1 antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 64, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 64.

In some embodiments, the anti-PD-1 antigen-binding domain can further include a linker sequence (e.g., any of the exemplary linker sequences described herein or known in the art) between the first sequence and the second sequence.

In some embodiments, the anti-PD-1 antigen-binding domain is an anti-PD-1 diabody. In some embodiments, an anti-PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 21.

In some embodiments, an anti-PD-1 diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 22.

In some embodiments, the anti-PD-1 antigen-binding domain is an anti-mouse PD-1 diabody. In some embodiments, an anti-mouse PD-1 diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 134.

In some embodiments, the anti-PD-1 antigen-binding domain is a human anti-PD-1 antibody. In some embodiments, the anti-PD-1 antigen-binding domain is a mouse anti-PD-1 antibody.

In some embodiments, the anti-PD-1 antigen-binding domain is an anti-PD-1 single chain antibody (e.g., an anti-PD-1 scFv). In some embodiments, the anti-PD-1 antigen-binding domain is a human anti-PD-1 single chain antibody. In some embodiments, the anti-human PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 135.

In some embodiments, the anti-PD-1 antigen-binding domain is a mouse anti-PD-1 single chain antibody. In some embodiments, the anti-mouse PD-1 scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 136.

A non-limiting example of a soluble TGFβRII receptor that binds specifically to a ligand of TGF-βRII can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7, and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor is encoded by a first nucleic acid encoding a first sequence at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 8, SEQ ID NO: 49, or SEQ ID NO: 50, and a second nucleic acid sequence encoding a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to: SEQ ID NO: 8, SEQ ID NO: 49, or SEQ ID NO: 50.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 51.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10 or SEQ ID NO: 52.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 53.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 15.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 16.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 129, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 130.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 131.

In some embodiments, the second chimeric polypeptide further comprises one or more additional target-binding domains at the N-terminal end or the C-terminal end of the second chimeric polypeptide. In some embodiments, the second chimeric polypeptide further comprises one or more additional target-binding domains at the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

In some embodiments, the additional target-binding domain includes or is soluble IL-7. In some embodiments, a soluble IL-7 can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 4.

Anti-Human TFscFv//TRβC/IL-15: Anti-PD-1 scFv/IL-15RαSu/IL-7

In some embodiments, provided herein is a multi-chain chimeric polypeptide that includes: (a) a first chimeric polypeptide including: (i) a first target-binding domain that includes an anti-human TFscFv (e.g., a sequence that is at least 80%, at least 90%, at least 95%, or 100% identical to SEQ ID NO: 131); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 scFv (e.g., a sequence that is at least 80%, at least 90%, at least 95%, or 100% identical to SEQ ID NO: 135 or SEQ ID NO: 136); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a soluble IL-7 (e.g., a sequence that is at least 80%, at least 90%, at least 95%, or 100% identical to SEQ ID NO: 4).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 183)
DIQMTQSPASLSASVGDRVTITCLASQTIDTWLAWYLQKPGKSPQLLIYAATNL
ADGVPSRFSGSGSGTDFSFTISSLQPEDFATYYCQQVYSSPFTFGQGTKLEIKG
GGGSGGGGSGGGGSQIQLVQSGGEVKKPGASVRVSCKASGYSFTDYNVYWVRQS
PGKGLEWIGYIDPYNGITIYDQNFKGKATLTVDKSTSTAYMELSSLRSEDTAVY
FCARDVTTALDFWGQGTTVTVSSGGGGSEDLNKVFPPEVAVFEPSEAEISHTQK
ATLVCLATGFFPDHVELSWWVNGKEVHSGVSTDPQPLKEQPALNDSRYCLSSRL
RVSATFWQNPRNHFRCQVQFYGLSENDEWTQDRAKPVTQIVSAEAWGRADNWVN
VISDLKKIEDLIQSMHIDATLYTESDVHPSCKVTAMKCFLLELQVISLESGDAS
IHDTVENLIILANNSLSSNGNVTESGCKECEELEEKNIKEFLQSFVHIVQMFIN
TS.

In some embodiments, the first chimeric polypeptide further includes a signal sequence at its N-terminal end. In some embodiments the signal peptide sequence is MGWSCIILFLVATATGVHS (SEQ ID NO: 139).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 184)
EIVLTQSPATLSLSPGERATLSCRASKGVSTSGYSYLHWYQQKPGQAPRLLIYL
ASYLESGVPARFSGSGSGTDFTLTISSLEPEDFAVYYCQHSRDLPLTFGGGTKV
EIKGGGGGGGGSGGGGSQVQLVQSGVEVKKPGASVKVSCKASGYTFTNYYMYW
VRQAPGQGLEWMGGINPSNGGTNFNEKFKNRVTLTTDSSTTTAYMELKSLQFDD
TAVYYCARRDYRFDMGFDYWGQGTTVTVSSGGGGSITCPPPMSVEHADIWVKSY
SLYSRERYICNSGFKRKAGTSSLTECVLNKATNVAHWTTPSLKCIRDCDIEGKD
GKQYESVLMVSDQLLDSMKEIGSNCLNNEFNFFKRHICDANKEGMELFRAARK
LRQFLKMNSTGDFDLHLLKVSEGTTILLNCTGQVKGRKPAALGEAQPTKSLEEN
KSLKEQKKLNDLCFLKRLLQEIKTCWNKILMGTKEH.

In some embodiments, the second chimeric polypeptide further includes a signal sequence at its N-terminal end. In some embodiments the signal peptide sequence is MGVKVLFALICIAVAEA (SEQ ID NO: 137).
Anti-Human TF(Diabody)/TRβC/IL-15: Anti-PD-1 scFv/IL-1SRαSu/IL-7

In some embodiments, provided herein is a multi-chain chimeric polypeptide that includes: (a) a first chimeric polypeptide including: (i) a first target-binding domain that includes an anti-human TF diabody (e.g., a sequence that is at least 80%, at least 90%, at least 95%, or 100% identical to SEQ ID NO: 15); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 scFv (e.g., a sequence that is at least 80%, at least 90%, at least 95%, or 100% identical to SEQ ID NO: 135 or SEQ ID NO: 136); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a soluble IL-7 (e.g., a sequence that is at least 80%, at least 90%, at least 95%, or 100% identical to SEQ ID NO: 4).

### TGFβRII/TRβC/IL-15: Anti-Human PD-1(Diabody)/IL-1SRαSu/IL-7

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human PD-1 diabody (e.g., a sequence that is at least 80%, at least 90%, at least 95%, or 100% identical to SEQ ID NO: 21); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a soluble IL-7.g., a sequence that is at least 80%, at least 90%, at least 95%, or 100% identical to SEQ ID NO: 4).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSNCSITSIC
EKPQEVCVAVWRKNDENITLETVCHDPKLPYHDFILEDAASPKCIMKEKKKPGE
TFFMCSCSSDECNDNIIFSEEYNTSNPDGGGGSIPPHVQKSVNNDMIVTDNNGA
VKFPQLCKFCDVRFSTCDNQKSCMSNCSITSICEKPQEVCVAVWRKNDENITLE
TVCHDPKLPYHDFILEDAASPKCIMKEKKKPGETFFMCSCSSDECNDNIIFSEE
YNTSNPDEDLNKVFPPEVAVFEPSEAEISHTQKATLVCLATGFFPDHVELSWWV
NGKEVHSGVSTDPQPLKEQPALNDSRYCLSSRLRVSATFWQNPRNHFRCQVQFY
GLSENDEWTQDRAKPVTQIVSAEAWGRADNWVNVISDLKKIEDLIQSMHIDATL
YTESDVHPSCKVTAMKCFLLELQVISLESGDASIHDTVENLIILANNSLSSNGN
VTESGCKECEELEEKNIKEFLQSFVHIVQMFINTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at 20 least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 185)
QVQLVQSGVEVKKPGASVKVSCKASGYTFTNYYMYWVRQAPGQGLEWMGGINPS
NGGTNFNEKFKNRVTLTTDSSTTTAYMELKSLQFDDTAVYYCARRDYREDMGFD
YWGQGTTVTVSSGGGGSEIVLTQSPATLSLSPGERATLSCRASKGVSTSGYSYL
HWYQQKPGQAPRLLIYLASYLESGVPARFSGSGSGTDFTLTISSLEPEDFAVYY
CQHSRDLPLTFGGGTKVEIKGGGGSGGGGSGGGGSQVQLVQSGVEVKKPGASVK
VSCKASGYTFTNYYMYWVRQAPGQGLEWMGGINPSNGGTNFNEKFKNRVTLTTD
SSTTTAYMELKSLQFDDTAVYYCARRDYRFDMGFDYWGQGTTVTVSSGGGGSEI
VLTQSPATLSLSPGERATLSCRASKGVSTSGYSYLHWYQQKPGQAPRLLIYLAS
YLESGVPARFSGSGSGTDFTLTISSLEPEDFAVYYCQHSRDLPLTFGGGTKVEI
KITCPPPMSVEHADIWVKSYSLYSRERYICNSGFKRKAGTSSLTECVLNKATNV
AHWTTPSLKCIRDCDIEGKDGKQYESVLMVSIDQLLDSMKEIGSNCLNNEFNFF
KRHICDANKEGMFLFRAARKLRQFLKMNSTGDFDLHLLKVSEGTTILLNCTGQV
KGRKPAALGEAQPTKSLEENKSLKEQKKLNDLCFLKRLLQEIKTCWNKILMGTK
EH.

In some embodiments, the second chimeric polypeptide further includes a signal sequence at its N-terminal end. In some embodiments the signal peptide sequence is MGVKVLFALICIAVAEA (SEQ ID NO: 137).

In some embodiments, the first chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 186)
ATCCCACCGCACGTTCAGAAGTCGGTTAATAACGACATGATAGTCACTGACAAC
AACGGTGCAGTCAAGTTTCCACAACTGTGTAAATTTTGTGATGTGAGATTTTCC
ACCTGTGACAACCAGAAATCCTGCATGAGCAACTGCAGCATCACCTCCATCTGT
GAGAAGCCACAGGAAGTCTGTGTGGCTGTATGGAGAAAGAATGACGAGAACATA
ACACTAGAGACAGTTTGCCATGACCCCAAGCTCCCCTACCATGACTTTATTCTG
GAAGATGCTGCTTCTCCAAAGTGCATTATGAAGGAAAAAAAAAAGCCTGGTGAG
ACTTTCTTCATGTGTTCCTGTAGCTCTGATGAGTGCAATGACAACATCATCTTC
TCAGAAGAATATAACACCAGCAATCCTGACGGCGGAGGTGGAAGTATCCCACCG
CACGTTCAGAAGTCGGTTAATAACGACATGATAGTCACTGACAACAACGGTGCA
GTCAAGTTTCCACAACTGTGTAAATTTTGTGATGTGAGATTTTCCACCTGTGAC
AACCAGAAATCCTGCATGAGCAACTGCAGCATCACCTCCATCTGTGAGAAGCCA
CAGGAAGTCTGTGTGGCTGTATGGAGAAAGAATGACGAGAACATAACACTAGAG
ACAGTTTGCCATGACCCCAAGCTCCCCTACCATGACTTTATTCTGGAAGATGCT
GCTTCTCCAAAGTGCATTATGAAGGAAAAAAAAAAGCCTGGTGAGACTTTCTTC
ATGTGTTCCTGTAGCTCTGATGAGTGCAATGACAACATCATCTTCTCAGAAGAA
TATAACACCAGCAATCCTGACGAGGACCTGAACAAGGTGTTCCCACCCGAGGTC
GCTGTGTTTGAGCCATCAGAAGCAGAGATCTCCCACACCCAAAAGGCCACACTG
GTGTGCCTGGCCACAGGCTTCTTCCCCGACCACGTGGAGCTGAGCTGGTGGGTG
AATGGGAAGGAGGTGCACAGTGGGGTCAGCACGGACCCGCAGCCCCTCAAGGAG
CAGCCCGCCCTCAATGACTCCAGATACTGCCTGAGCAGCCGCCTGAGGGTCTCG
GCCACCTTCTGGCAGAACCCCCGCAACCACTTCCGCTGTCAAGTCCAGTTCTAC
GGGCTCTCGGAGAATGACGAGTGGACCCAGGATAGGGCCAAACCCGTCACCCAG
ATCGTCAGCGCCGAGGCCTGGGGTAGAGCAGACAACTGGGTGAATGTAATAAGT
GATTTGAAAAAAATTGAAGATCTTATTCAATCTATGCATATTGATGCTACTTTA
TATACGGAAAGTGATGTTCACCCCAGTTGCAAAGTAACAGCAATGAAGTGCTTT
CTCTTGGAGTTACAAGTTATTTCACTTGAGTCCGGAGATGCAAGTATTCATGAT
ACAGTAGAAAATCTGATCATCCTAGCAAACAACAGTTTGTCTTCTAATGGGAAT
GTAACAGAATCTGGATGCAAAGAATGTGAGGAACTGGAGGAAAAAAATATTAAA
GAATTTTTGCAGAGTTTTGTACATATTGTCCAAATGTTCATCAACACTTCTTGA
TAACGCGTACGAAG.

In some embodiments, the second chimeric polypeptide is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to:

(SEQ ID NO: 187)
CAAGTTCAACTCGTGCAGAGCGGTGTGGAAGTTAAAAAGCCCGGCGCTAGCGTA
AAGGTGAGCTGTAAGGCTAGCGGCTACACCTTCACCAATTACTACATGTACTGG
GTTAGGCAAGCCCCTGGACAAGGCTTAGAATGGATGGGTGGGATAAACCCTTCG
AATGGTGGAACCAACTTTAACGAGAAGTTCAAAAACAGAGTGACGTTGACCACC
GACTCTAGCACCACTACCGCGTACATGGAACTGAAGAGCCTGCAGTTCGATGAT
ACAGCGGTGTATTATTGCGCCCGAAGAGACTACCGATTCGACATGGGGTTTGAC
TACTGGGGCCAAGGCACCACCGTCACCGTCAGCAGCGGCGGAGGCGGTAGTGAG
ATTGTTCTGACCCAAAGCCCTGCCACCCTCTCGTTAAGCCCGGGGGAGAGAGCA
ACCCTGAGCTGTAGAGCTAGCAAGGGCGTGAGCACCTCGGGCTACAGTTACCTG
CACTGGTACCAACAGAAGCCTGGCCAAGCCCCTCGATTATTGATCTACTTAGCT
AGCTACTTAGAAAGCGGCGTGCCCGCTAGATTCAGCGGCAGCGGGAGCGGCACC
GACTTTACACTGACGATAAGTAGCTTAGAGCCTGAGGACTTTGCAGTGTATTAC
TGCCAACATTCCCGGGACCTGCCGCTTACCTTCGGGGGCGGCACCAAAGTGGAG
ATTAAAGGAGGCGGAGGGTCGGGCGGTGGTGGGTCTGGAGGTGGAGGAAGCCAA
GTGCAGCTGGTGCAGAGCGGCGTGGAGGTGAAGAAGCCGGGCGCTAGCGTGAAG
GTGAGCTGCAAGGCCTCCGGATATACCTTCACAAACTACTACATGTATTGGGTT
AGACAAGCCCCTGGTCAAGGCCTGGAGTGGATGGGCGGTATAAACCCTTCCAAT
GGAGGTACTAATTTCAACGAAAAATTCAAGAACCGTGTGACCCTGACCACCGAC
AGTAGCACCACCACTGCATACATGGAGCTGAAGAGTCTGCAGTTCGACGATACC
GCGGTCTACTACTGTGCCCGACGAGACTATAGATTTGACATGGGCTTCGACTAC
TGGGGACAAGGCACTACGGTGACCGTGAGCAGCGGTGGCGGCGGCTCCGAAATT
GTGCTGACACAGTCGCCTGCAACCCTGTCCTTGAGCCCTGGCGAGCGGGCAACT
CTGAGCTGCCGAGCTAGCAAAGGCGTGAGTACAAGCGGCTATAGCTACCTACAT
TGGTATCAGCAAAAGCCTGGCCAAGCCCCGCGACTGCTGATCTACCTGGCTAGC
TACCTGGAGAGCGGCGTTCCTGCTAGATTCAGCGGATCTGGCTCTGGTACTGAC
TTCACCTTAACTATCAGTAGCCTTGAACCCGAGGACTTCGCTGTGTACTATTGT
CAGCACAGCAGAGACCTCCCTCTGACCTTTGGAGGTGGTACCAAGGTCGAAATC
AAAATCACGTGCCCTCCCCCCATGTCCGTGGAACACGCAGACATCTGGGTCAAG
AGCTACAGCTTGTACTCCAGGGAGCGGTACATTTGTAACTCTGGTTTCAAGCGT
AAAGCCGGCACGTCCAGCCTGACGGAGTGCGTGTTGAACAAGGCCACGAATGTC
GCCCACTGGACAACCCCCAGTCTCAAATGCATTAGAGATTGTGATATTGAAGGT
AAAGATGGCAAACAATATGAGAGTGTTCTAATGGTCAGCATCGATCAATTATTG
GACAGCATGAAAGAAATTGGTAGCAATTGCCTGAATAATGAATTTAACTTTTTT
AAAAGACATATCTGTGATGCTAATAAGGAAGGTATGTTTTTATTCCGTGCTGCT
CGCAAGTTGAGGCAATTTCTTAAAATGAATAGCACTGGTGATTTTGATCTCCAC
TTATTAAAAGTTTCAGAAGGCACAACAATACTGTTGAACTGCACTGGCCAGGTT
AAAGGAAGAAAACCAGCTGCCCTGGGTGAAGCCCAACCAACAAAGAGTTTGGAA
GAAAATAAATCTTTAAAGGAACAGAAAAAACTGAATGACTTGTGTTTCCTAAAG
AGACTATTACAAGAGATAAAAACTTGTTGGAATAAAATTTTGATGGGCACTAAA
GAACACTGA.

### TGFβRII/TRβC/IL-15: Anti-mPD-1(Diabody)/IL-1SRαSu/IL-7

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-mouse PD-1 diabody (e.g., a sequence that is at least 80%, at least 90%, at least 95%, or 100% identical to SEQ ID NO: 134); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a human soluble IL-7 (e.g., a sequence that is at least 80%, at least 90%, at least 95%, or 100% identical to SEQ ID NO: 4).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPDGGGGS
IPPHVQKSVNNDMIVTDNNGAVKFPQLCKFCDVRFSTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPDEDLNK
VFPPEVAVFEPSEAEISHTQKATLVCLATGFFPDHVELSWWVNGKEV
HSGVSTDPQPLKEQPALNDSRYCLSSRLRVSATFWQNPRNHERCQVQ
FYGLSENDEWTQDRAKPVTQIVSAEAWGRADNWVNVISDLKKIEDLI
QSMHIDATLYTESDVHPSCKVTAMKCFLLELQVISLESGDASIHDTV
ENLIILANNSLSSNGNVTESGCKECEELEEKNIKEFLQSFVHIVQMF
INTS

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 188)
QVQLQQSGAELVKPGSSVKISCKASGYTFTSHFIHWIKQQPGNGLEW
IGGIYPGDGDTEYNQQFNGKATLTADKSSSTAYMRLSSLTSEDSAVY
FCATRVPSYWFFDEWGPGTMVTVSSGGGGSRTDVALTQTPVAQPVTL
GDQASISCRSSQSLVHSNGRTYLEWYLQKPGQSPQLLIYKVSNRFSG
VPDRFIGSGSGSDFTLTISRVEPEDLGVYYCFQATHDPNTFGAGTKL
ELKGGGGSGGGGGGGGSQVQLQQSAAELVKPGSSVKISCKASGYTFT
SHFIHWIKQQPGNGLEWIGGIYPGDGDTEYNQQENGKATLTADKSSS
TAYMRLSSLTSEDSAVYFCATRVPSYWFFDFWGPGTMVTVSSGGGGS
RTDVALTQTPVAQPVTLGDQASISCRSSQSLVHSNGRTYLEWYLQKP
GQSPQLLIYKVSNRFSGVPDRFIGSGSGSDFTLTISRVEPEDLGVYY
CFQATHDPNTFGAGTKLELKITCPPPMSVEHADIWVKSYSLYSRERY
ICNSGFKRKAGTSSLTECVLNKATNVAHWTTPSLKCIRDCDIEGKDG
KQYESVLMVSIDQLLDSMKEIGSNCLNNEFNFFKRHICDANKEGMFL
FRAARKLRQFLKMNSTGDFDLHLLKVSEGTTILLNCTGQVKGRKPAA
LGEAQPTKSLEENKSLKEQKKLNDLCFLKRLLQEIKTCWNKILMGTK
EH.

In some embodiments, the second chimeric polypeptide further includes a signal sequence at its N-terminal end. In some embodiments the signal peptide sequence is MGVKVLFALICIAVAEA (SEQ ID NO: 137).
TGFβRII/TRβC/IL-15: Anti-PD-1 scFv/IL-15RαSu/IL-7

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 scFv (e.g., a sequence that is at least 80%, at least 90%, at least 95%, or 100% identical to SEQ ID NO: 135 or SEQ ID NO: 136); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a soluble IL-7 (e.g., a sequence that is at least 80%, at least 90%, at least 95%, or 100% identical to SEQ ID NO: 4).

### TGFβRII/TRβC/IL-15: Anti-PD-1(Diabody)/IL-1SRαSu

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 diabody (e.g., a sequence that is at least 80%, at least 90%, at least 95%, or 100% identical to SEQ ID NO: 21 or SEQ ID NO: 134); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPDGGGGS
IPPHVQKSVNNDMIVTDNNGAVKFPQLCKFCDVRFSTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPDEDLNK
VFPPEVAVFEPSEAEISHTQKATLVCLATGFFPDHVELSWWVNGKEV
HSGVSTDPQPLKEQPALNDSRYCLSSRLRVSATFWQNPRNHFRCQVQ
FYGLSENDEWTQDRAKPVTQIVSAEAWGRADNWVNVISDLKKIEDLI
QSMHIDATLYTESDVHPSCKVTAMKCFLLELQVISLESGDASIHDTV
ENLIILANNSLSSNGNVTESGCKECEELEEKNIKEFLQSFVHIVQMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 182)
QVQLVQSGVEVKKPGASVKVSCKASGYTFTNYYMYWVRQAPGQGLEW
MGGINPSNGGTNFNEKFKNRVTLTTDSSTTTAYMELKSLQFDDTAVY
YCARRDYRFDMGFDYWGQGTTVTVSSGGGGSEIVLTQSPATLSLSPG
ERATLSCRASKGVSTSGYSYLHWYQQKPGQAPRLLIYLASYLESGVP
ARFSGSGSGTDFTLTISSLEPEDFAVYYCQHSRDLPLTFGGGTKVEI
KGGGGSGGGGSGGGGSQVQLVQSGVEVKKPGASVKVSCKASGYTFTN
YYMYWVRQAPGQGLEWMGGINPSNGGTNFNEKFKNRVTLTTDSSTTT
AYMELKSLQFDDTAVYYCARRDYRFDMGFDYWGQGTTVTVSSGGGGS
EIVLTQSPATLSLSPGERATLSCRASKGVSTSGYSYLHWYQQKPGQA
PRLLIYLASYLESGVPARFSGSGSGTDFTLTISSLEPEDFAVYYCQH
SRDLPLTFGGGTKVEIKITCPPPMSVEHADIWVKSYSLYSRERYICN
SGFKRKAGTSSLTECVLNKATNVAHWTTPSLKCIR.

In some embodiments, the second chimeric polypeptide further includes a signal sequence at its N-terminal end. In some embodiments the signal peptide sequence is MGVKVLFALICIAVAEA (SEQ ID NO: 137).
TGFβRII/TRβC/IL-15: Anti-PD-1 scFv/IL-15RαSu

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 scFv (e.g., a sequence that is at least 80%, at least 90%, at least 95%, or 100% identical to SEQ ID NO: 135 or SEQ ID NO: 136); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

### Anti-TFscFv/TRβC/IL-15: Anti-hPD-1(diabody)/IL-15RαSu/IL-7

In some embodiments, provided herein is a multi-chain chimeric polypeptide that includes: (a) a first chimeric polypeptide including: (i) a first target-binding domain that includes an anti-TFscFv (e.g., a sequence that is at least 80%, at least 90%, at least 95%, or 100% identical to SEQ ID NO: 131); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human PD-1 diabody (e.g., a sequence that is at least 80%, at least 90%, at least 95%, or 100% identical to SEQ ID NO: 21); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a human soluble IL-7 (e.g., a sequence that is at least 80%, at least 90%, at least 95%, or 100% identical to SEQ ID NO: 4).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 183)
DIQMTQSPASLSASVGDRVTITCLASQTIDTWLAWYLQKPGKSPQLL
IYAATNLADGVPSRFSGSGSGTDESFTISSLQPEDFATYYCQQVYSS
PFTFGQGTKLEIKGGGGSGGGGSGGGGSQIQLVQSGGEVKKPGASVR
VSCKASGYSFTDYNVYWVRQSPGKGLEWIGYIDPYNGITIYDQNFKG
KATLTVDKSTSTAYMELSSLRSEDTAVYFCARDVTTALDFWGQGTTV
TVSSGGGGSEDLNKVFPPEVAVFEPSEAEISHTQKATLVCLATGFFP
DHVELSWWVNGKEVHSGVSTDPQPLKEQPALNDSRYCLSSRLRVSAT
FWQNPRNHFRCQVQFYGLSENDEWTQDRAKPVTQIVSAEAWGRADNW
VNVISDLKKIEDLIQSMHIDATLYTESDVHPSCKVTAMKCFLLELQV
ISLESGDASIHDTVENLIILANNSLSSNGNVTESGCKECEELEEKNI
KEFLQSFVHIVQMFINTS.

In some embodiments, the first chimeric polypeptide further includes a signal sequence at its N-terminal end. In some embodiments the signal peptide sequence is MGWSCIILFLVATATGVHS (SEQ ID NO: 139).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 185)
QVQLVQSGVEVKKPGASVKVSCKASGYTFTNYYMYWVRQAPGQGLEW
MGGINPSNGGTNFNEKFKNRVTLTTDSSTTTAYMELKSLQFDDTAVY
YCARRDYRFDMGEDYWGQGTTVTVSSGGGGSEIVLTQSPATLSLSPG
ERATLSCRASKGVSTSGYSYLHWYQQKPGQAPRLLIYLASYLESGVP
ARFSGSGSGTDFTLTISSLEPEDFAVYYCQHSRDLPLTFGGGTKVEI
KGGGGSGGGGSGGGGSQVQLVQSGVEVKKPGASVKVSCKASGYTFTN
YYMYWVRQAPGQGLEWMGGINPSNGGTNFNEKFKNRVTLTTDSSTTT
AYMELKSLQFDDTAVYYCARRDYRFDMGEDYWGQGTTVTVSSGGGGS
EIVLTQSPATLSLSPGERATLSCRASKGVSTSGYSYLHWYQQKPGQA
PRLLIYLASYLESGVPARFSGSGSGTDFTLTISSLEPEDFAVYYCQH
SRDLPLTFGGGTKVEIKITCPPPMSVEHADIWVKSYSLYSRERYICN
SGFKRKAGTSSLTECVLNKATNVAHWTTPSLKCIRDCDIEGKDGKQY
ESVLMVSIDQLLDSMKEIGSNCLNNEFNFFKRHICDANKEGMFLFRA
ARKLRQFLKMNSTGDFDLHLLKVSEGTTILLNCTGQVKGRKPAALGE
AQPTKSLEENKSLKEQKKLNDLCFLKRLLQEIKTCWNKILMGTKEH.

In some embodiments, the second chimeric polypeptide further includes a signal sequence at its N-terminal end. In some embodiments the signal peptide sequence is MGVKVLFALICIAVAEA (SEQ ID NO: 137).

### Anti-TFscFv/TRβC/IL-15: Anti-mPD-1(diabody)/IL-15RαSu/IL-7

In some embodiments, provided herein is a multi-chain chimeric polypeptide that includes: (a) a first chimeric polypeptide including: (i) a first target-binding domain that includes an anti-TFscFv (e.g., a sequence that is at least 80%, at least 90%, at least 95%, or 100% identical to SEQ ID NO: 131); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-mouse PD-1 diabody (e.g., a sequence that is at least 80%, at least 90%, at least 95%, or 100% identical to SEQ ID NO: 134); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a human soluble IL-7 (e.g., a sequence that is at least 80%, at least 90%, at least 95%, or 100% identical to SEQ ID NO: 4).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 183)
DIQMTQSPASLSASVGDRVTITCLASQTIDTWLAWYLQKPGKSPQLL
IYAATNLADGVPSRFSGSGSGTDESFTISSLQPEDFATYYCQQVYSS
PFTFGQGTKLEIKGGGGSGGGGSGGGGSQIQLVQSGGEVKKPGASVR
VSCKASGYSFTDYNVYWVRQSPGKGLEWIGYIDPYNGITIYDQNFKG
KATLTVDKSTSTAYMELSSLRSEDTAVYFCARDVTTALDFWGQGTTV
TVSSGGGGSEDLNKVFPPEVAVFEPSEAEISHTQKATLVCLATGFFP
DHVELSWWVNGKEVHSGVSTDPQPLKEQPALNDSRYCLSSRLRVSAT
FWQNPRNHFRCQVQFYGLSENDEWTQDRAKPVTQIVSAEAWGRADNW
VNVISDLKKIEDLIQSMHIDATLYTESDVHPSCKVTAMKCFLLELQV
ISLESGDASIHDTVENLIILANNSLSSNGNVTESGCKECEELEEKNI
KEFLQSFVHIVQMFINTS.

In some embodiments, the first chimeric polypeptide further includes a signal sequence at its N-terminal end. In some embodiments the signal peptide sequence is MGWSCIILFLVATATGVHS (SEQ ID NO: 139).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 188)
QVQLQQSGAELVKPGSSVKISCKASGYTFTSHFIHWIKQQPGNGLEW
IGGIYPGDGDTEYNQQFNGKATLTADKSSSTAYMRLSSLTSEDSAVY
FCATRVPSYWFFDEWGPGTMVTVSSGGGGSRTDVALTQTPVAQPVTL
GDQASISCRSSQSLVHSNGRTYLEWYLQKPGQSPQLLIYKVSNRFSG
VPDRFIGSGSGSDFTLTISRVEPEDLGVYYCFQATHDPNTFGAGTKL
ELKGGGGSGGGGSGGGGSQVQLQQSAAELVKPGSSVKISCKASGYTF
TSHFIHWIKQQPGNGLEWIGGIYPGDGDTEYNQQFNGKATLTADKSS
STAYMRLSSLTSEDSAVYFCATRVPSYWFFDFWGPGTMVTVSSGGGG
SRTDVALTQTPVAQPVTLGDQASISCRSSQSLVHSNGRTYLEWYLQK
PGQSPQLLIYKVSNRFSGVPDRFIGSGSGSDFTLTISRVEPEDLGVY
YCFQATHDPNTFGAGTKLELKITCPPPMSVEHADIWVKSYSLYSRER
YICNSGFKRKAGTSSLTECVLNKATNVAHWTTPSLKCIRDCDIEGKD
GKQYESVLMVSIDQLLDSMKEIGSNCLNNEFNFFKRHICDANKEGMF
LFRAARKLRQFLKMNSTGDFDLHLLKVSEGTTILLNCTGQVKGRKPA
ALGEAQPTKSLEENKSLKEQKKLNDLCFLKRLLQEIKTCWNKILMGT
KEH.

In some embodiments, the second chimeric polypeptide further includes a signal sequence at its N-terminal end. In some embodiments the signal peptide sequence is MGVKVLFALICIAVAEA (SEQ ID NO: 137).

### Anti-TF(Diabody)/TRβC/IL-15: Anti-hPD-1(Diabody)/IL-15RαSu/IL-7

In some embodiments, provided herein is a multi-chain chimeric polypeptide that includes: (a) a first chimeric polypeptide including: (i) a first target-binding domain that includes an TF diabody (e.g., a sequence that is at least 80%, at least 90%, at least 95%, or 100% identical to SEQ ID NO: 15); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-human PD-1 diabody (e.g., a sequence that is at least 80%, at least 90%, at least 95%, or 100% identical to SEQ ID NO: 21); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes a human soluble IL-7 (e.g., a sequence that is at least 80%, at least 90%, at least 95%, or 100% identical to SEQ ID NO: 4).

Anti-TF scFv/TRβC/IL-15: Anti-PD-1(Diabody)/IL-1SRαSu

In some embodiments, provided herein is a multi-chain chimeric polypeptide that includes: (a) a first chimeric polypeptide including: (i) a first target-binding domain that includes an anti-TFscFv (e.g., a sequence that is at least 80%, at least 90%, at least 95%, or 100% identical to SEQ ID NO: 131); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-PD-1 diabody (e.g., a sequence that is at least 80%, at least 90%, at least 95%, or 100% identical to SEQ ID NO: 21 or SEQ ID NO: 134); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 183)
DIQMTQSPASLSASVGDRVTITCLASQTIDTWLAWYLQKPGKSPQLL
IYAATNLADGVPSRFSGSGSGTDFSFTISSLQPEDFATYYCQQVYSS
PFTFGQGTKLEIKGGGGSGGGGSGGGGSQIQLVQSGGEVKKPGASVR
VSCKASGYSFTDYNVYWVRQSPGKGLEWIGYIDPYNGITIYDQNFKG
KATLTVDKSTSTAYMELSSLRSEDTAVYFCARDVTTALDFWGQGTTV
TVSSGGGGSEDLNKVFPPEVAVFEPSEAEISHTQKATLVCLATGFFP
DHVELSWWVNGKEVHSGVSTDPQPLKEQPALNDSRYCLSSRLRVSAT
FWQNPRNHFRCQVQFYGLSENDEWTQDRAKPVTQIVSAEAWGRADNW
VNVISDLKKIEDLIQSMHIDATLYTESDVHPSCKVTAMKCFLLELQV
ISLESGDASIHDTVENLIILANNSLSSNGNVTESGCKECEELEEKNI
KEFLQSFVHIVQMFINTS.

In some embodiments, the first chimeric polypeptide further includes a signal sequence at its N-terminal end. In some embodiments the signal peptide sequence is MGWSCIILFLVATATGVHS (SEQ ID NO: 139).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 182)
QVQLVQSGVEVKKPGASVKVSCKASGYTFTNYYMYWVRQAPGQGLEW
MGGINPSNGGTNFNEKFKNRVTLTTDSSTTTAYMELKSLQFDDTAVY
YCARRDYRFDMGEDYWGQGTTVTVSSGGGGSEIVLTQSPATLSLSPG
ERATLSCRASKGVSTSGYSYLHWYQQKPGQAPRLLIYLASYLESGVP
ARFSGSGSGTDFTLTISSLEPEDFAVYYCQHSRDLPLTFGGGTKVEI
KGGGGSGGGGSGGGGSQVQLVQSGVEVKKPGASVKVSCKASGYTFTN
YYMYWVRQAPGQGLEWMGGINPSNGGTNFNEKFKNRVTLTTDSSTTT
AYMELKSLQFDDTAVYYCARRDYRFDMGEDYWGQGTTVTVSSGGGGS
EIVLTQSPATLSLSPGERATLSCRASKGVSTSGYSYLHWYQQKPGQA
PRLLIYLASYLESGVPARFSGSGSGTDFTLTISSLEPEDFAVYYCQH
SRDLPLTFGGGTKVEIKITCPPPMSVEHADIWVKSYSLYSRERYICN
SGFKRKAGTSSLTECVLNKATNVAHWTTPSLKCIR.

In some embodiments, the second chimeric polypeptide further includes a signal sequence at its N-terminal end. In some embodiments the signal peptide sequence is MGVKVLFALICIAVAEA (SEQ ID NO: 137).

### Exemplary Multi-Chain Chimeric Polypeptides—Type H

In some embodiments of any of the multi-chain chimeric polypeptides described herein, the first target-binding domain and the second targeting-binding domain each independently bind specifically to tissue factor or to CD16a. In some embodiments of these multi-chain chimeric polypeptides, the first target-binding domain and the second target-binding domain both bind specifically to tissue factor or to CD16a.

In some embodiments of these multi-chain chimeric polypeptides, the first target-binding domain binds specifically to tissue factor, and the second target-binding domain binds specifically to CD16a. In some embodiments of these multi-chain chimeric polypeptides, the first target-binding domain binds specifically to CD16a, and the second target-binding domain bind specifically to tissue factor.

In some examples of these multi-chain chimeric polypeptides, the first target-binding domain and the soluble TRβC domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble TRβC domain in the first chimeric polypeptide.

In some embodiments of these multi-chain chimeric polypeptides, the soluble TRβC domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble TRβC domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble T cell receptor β-chain constant region (TRβC) domain can be any of the exemplary soluble T cell receptor β-chain constant region (TRβC) domains described herein.

In some embodiments of these multi-chain chimeric polypeptides, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these multi-chain chimeric polypeptides, the pair of affinity domains can be any of the exemplary pairs of affinity domains described herein. In some embodiments, the pair of affinity domains is a sushi domain from an alpha chain of human IL-15 receptor (IL-15Rα) and a soluble IL-15. In some embodiments, the second domain of the pair of affinity domains is a sushi domain from an alpha chain of human IL-15 receptor (IL-15Rα) and the first domain of the pair of affinity domains a soluble IL-15. In some embodiments, the first domain of the pair of affinity domains is a sushi domain from an alpha chain of human IL-15 receptor (IL-15Rα) and the second domain of the pair of affinity domains a soluble IL-15.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 11.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 115.

In some embodiments, a soluble IL-15 can include a sequence that is at least 70% identical, at least 75% identical, at least 80% identical, at least 85% identical, at least 90% identical, at least 95% identical, at least 99% identical, or 100% identical to SEQ ID NO: 13.

In some embodiments of these multi-chain chimeric polypeptides, one or both of the first target-binding domain and the second target-binding domain is an anti-tissue factor antigen-binding domain or an anti-CD16a antigen-binding domain. In some embodiments of these multi-chain chimeric polypeptides, the first target-binding domain and the second target-binding domain are each independently an anti-tissue factor antigen-binding domain or an anti-CD16a antigen-binding domain. In some embodiments of these multi-chain chimeric polypeptides, the first target-binding domain is an anti-tissue factor antigen-binding domain and the second target-binding domain is an anti-CD16a antigen-binding domain. In some embodiments of these multi-chain chimeric polypeptides, the first target-binding domain is an anti-CD16a antigen-binding domain and the second target-binding domain is an anti-tissue factor antigen-binding domain.

In some embodiments, an anti-tissue factor antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53.

In some embodiments, an anti-tissue factor antigen-binding domain can include a first sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 53, and a second sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 53.

In some embodiments, the anti-tissue factor antigen-binding domain can further include a linker sequence (e.g., any of the exemplary linker sequences described herein or known in the art) between the first sequence and the second sequence.

In some embodiments an anti-tissue factor antigen-binding domain is an anti-tissue factor diabody. In some embodiments, an anti-tissue factor diabody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 15.

In some embodiments, an anti-tissue factor diabody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 16.

In some embodiments, an anti-tissue factor antigen-binding domain can include a heavy sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 129, and a light chain sequence that is at least 70% identical (e.g., at least 75% identical, at least 80% identical, at least 85% identical, at least 90% identical, at least 92% identical, at least 95% identical, at least 96% identical, at least 98% identical, or 100% identical) to SEQ ID NO: 130.

In some embodiments, the anti-tissue factor antigen-binding domain is an anti-tissue factor scFv. In some embodiments, the anti-tissue factor antigen-binding domain is a humanized anti-human tissue factor scFv. In some embodiments, the humanized anti-human tissue factor scFv includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 131.

In some embodiments, the anti-CD16a antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 119.

In some embodiments, the second chimeric polypeptide further comprises one or more additional target-binding domains at the N-terminal end or the C-terminal end of the second chimeric polypeptide. In some embodiments, the second chimeric polypeptide further comprises one or more additional target-binding domains at the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

### Humanized Anti-Human TFscFv/TRβC/IL-15: Anti-CD16a-scFv/IL-15RαSu

In some embodiments, provided herein is a multi-chain chimeric polypeptide that includes: (a) a first chimeric polypeptide including: (i) a first target-binding domain that includes a humanized anti-TFscFv (e.g., a sequence that is at least 80%, at least 90%, at least 95%, or 100% identical to SEQ ID NO: 131); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes an anti-CD16a-scFv (e.g., a sequence that is at least 80%, at least 90%, at least 95%, or 100% identical to SEQ ID NO: 119); and (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 183)
DIQMTQSPASLSASVGDRVTITCLASQTIDTWLAWYLQKPGKSPQLL
IYAATNLADGVPSRESGSGSGTDFSFTISSLQPEDFATYYCQQVYSS
PFTFGQGTKLEIKGGGGSGGGGSGGGGSQIQLVQSGGEVKKPGASVR
VSCKASGYSFTDYNVYWVRQSPGKGLEWIGYIDPYNGITIYDQNFKG
KATLTVDKSTSTAYMELSSLRSEDTAVYFCARDVTTALDFWGQGTTV
TVSSGGGGSEDLNKVFPPEVAVFEPSEAEISHTQKATLVCLATGFFP
DHVELSWWVNGKEVHSGVSTDPQPLKEQPALNDSRYCLSSRLRVSAT
FWQNPRNHFRCQVQFYGLSENDEWTQDRAKPVTQIVSAEAWGRADNW
VNVISDLKKIEDLIQSMHIDATLYTESDVHPSCKVTAMKCFLLELQV
ISLESGDASIHDTVENLIILANNSLSSNGNVTESGCKECEELEEKNI
KEFLQSFVHIVQMFINTS.

In some embodiments, the first chimeric polypeptide further includes a signal sequence at its N-terminal end. In some embodiments the signal peptide sequence is MGWSCIILFLVATATGVHS (SEQ ID NO: 139).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 189)
SELTQDPAVSVALGQTVRITCQGDSLRSYYASWYQQKPGQAPVLVIY
GKNNRPSGIPDRFSGSSSGNTASLTITGAQAEDEADYYCNSRDSSGN
HVVFGGGTKLTVGHGGGGSGGGGSGGGGSEVQLVESGGGVVRPGGSL
RLSCAASGFTEDDYGMSWVRQAPGKGLEWVSGINWNGGSTGYADSVK
GRFTISRDNAKNSLYLQMNSLRAEDTAVYYCARGRSLLFDYWGQGTL
VTVSRITCPPPMSVEHADIWVKSYSLYSRERYICNSGFKRKAGTSSL
TECVLNKATNVAHWTTPSLKCIR.

In some embodiments, the second chimeric polypeptide further includes a signal sequence at its N-terminal end. In some embodiments the signal peptide sequence is MGWSCIILFLVATATGVHS (SEQ ID NO: 139).

### Exemplary Multi-Chain Chimeric Polypeptides—Type I

In some embodiments of any of the multi-chain chimeric polypeptides described herein, the first target-binding domain and the second targeting-binding domain each independently bind specifically to a ligand of TGF-βRII or to HIV. In some embodiments of these multi-chain chimeric polypeptides, the first target-binding domain and the second target-binding domain both bind specifically to a ligand of TGF-βRII or to HIV.

In some embodiments of these multi-chain chimeric polypeptides, the first target-binding domain binds specifically to a ligand of TGF-βRII, and the second target-binding domain binds specifically to HIV. In some embodiments of these multi-chain chimeric polypeptides, the first target-binding domain binds specifically to HIV, and the second target-binding domain binds specifically to a ligand of TGF-βRII.

In some examples of these multi-chain chimeric polypeptides, the first target-binding domain and the soluble TRβC domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble TRβC domain in the first chimeric polypeptide.

In some embodiments of these multi-chain chimeric polypeptides, the soluble TRβC domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble TRβC domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble T cell receptor β-chain constant region (TRβC) domain can be any of the exemplary soluble T cell receptor β-chain constant region (TRβC) domains described herein.

In some embodiments of these multi-chain chimeric polypeptides, the soluble T cell receptor β-chain constant region (TRβC) domain includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 1 or 3.

In some embodiments of these multi-chain chimeric polypeptides, the pair of affinity domains can be any of the exemplary pairs of affinity domains described herein. In some embodiments, the pair of affinity domains is a sushi domain from an alpha chain of human IL-15 receptor (IL-15Rα) and a soluble TL-15. In some embodiments, the second domain of the pair of affinity domains is a sushi domain from an alpha chain of human IL-15 receptor (IL-15Rα) and the first domain of the pair of affinity domains a soluble IL-15. In some embodiments, the first domain of the pair of affinity domains is a sushi domain from an alpha chain of human IL-15 receptor (IL-15Rα) and the second domain of the pair of affinity domains a soluble IL-15.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 11.

In some embodiments, the sushi domain from an alpha chain of IL-15 receptor alpha (IL-15Rα) can include a sequence that is at least 70% identical, at least 75% identical, at least 80% identical, at least 85% identical, at least 90% identical, at least 95% identical, at least 99% identical, or 100% identical to SEQ ID NO: 115.

In some embodiments, a soluble IL-15 can include a sequence that is at least 70% identical, at least 75% identical, at least 80% identical, at least 85% identical, at least 90% identical, at least 95% identical, at least 99% identical, or 100% identical to SEQ ID NO: 13.

In some embodiments of these multi-chain chimeric polypeptides, one or both of the first target-binding domain and the second target-binding domain is a soluble TGFβRII receptor or an anti-HIV antigen-binding domain. In some embodiments of these multi-chain chimeric polypeptides, the first target-binding domain and the second target-binding domain are each independently a soluble TGFβRII receptor or an anti-HIV antigen-binding domain. In some embodiments of these multi-chain chimeric polypeptides, the first target-binding domain is a soluble TGFβRII receptor and the second target-binding domain is an anti-HIV antigen-binding domain. In some embodiments of these multi-chain chimeric polypeptides, the first target-binding domain is an anti-HIV antigen-binding domain and the second target-binding domain is a soluble TGFβRII receptor.

A non-limiting example of a soluble TGFβRII receptor that binds specifically to a ligand of TGF-βRII can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor can include a first sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7, and a second sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 7.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 9 or SEQ ID NO: 51.

In some embodiments of these multi-chain chimeric polypeptides, a soluble TGFβRII receptor can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 10 or SEQ ID NO: 52.

In some embodiments, the anti-HIV antigen-binding domain can include a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 121.

In some embodiments, the second chimeric polypeptide further comprises one or more additional target-binding domains at the N-terminal end or the C-terminal end of the second chimeric polypeptide. In some embodiments, the second chimeric polypeptide further comprises one or more additional target-binding domains at the C-terminal end of the second chimeric polypeptide.

In some embodiments of these multi-chain chimeric polypeptides, the additional target-binding domain and the second domain of the pair of affinity domains directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the additional target-binding domain in the second chimeric polypeptide.

In some embodiments, the additional target-binding domain includes or is an anti-CD3 antigen-binding domain.

In some embodiments, the additional target-binding domain includes or is a mouse anti-CD3 antigen-binding domain. In some embodiments, the additional target-binding domain includes or is a human anti-CD3 antigen-binding domain.

In some embodiments, the additional target-binding domain includes or is a single-chain anti-CD3 antibody. A non-limiting example of a scCD3 antibody includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to SEQ ID NO: 17.

In some embodiments, a scCD3 antibody can be encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 18.

In some embodiments, a scCD3 antibody includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 19.

In some embodiments, a scCD3 antibody is encoded by a nucleic acid including a sequence at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 20.

In some embodiments, the additional target-binding domain includes or is an anti-CD3 scFv. In some embodiments, the anti-CD3 scFv can include a heavy chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 54 and/or a light chain variable domain including a sequence that is at least 70% identical (e.g., at least 75% identical, at least 80% identical, at least 85% identical, at least 90% identical, at least 95% identical, at least 99% identical, or 100% identical) to SEQ ID NO: 55.

In some embodiments, an anti-CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 58.

In some embodiments, the anti-CD3 scFv can be an anti-mouse CD3 scFv. In some embodiments, an anti-mouse CD3 scFv can include a sequence that is at least 70% identical (e.g., at least 75% identical, at least 80% identical, at least 85% identical, at least 90% identical, at least 95% identical, at least 99% identical, or 100% identical) to SEQ ID NO: 133.

In some embodiments, the additional target-binding domain includes or is an anti-CD3 antibody. In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 60 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 61.

In some embodiments, the anti-CD3 antibody can include a heavy chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 62 and/or a light chain that includes a sequence that is at least 70% identical (e.g., at least 75% identical, at least 80% identical, at least 85% identical, at least 90% identical, at least 92% identical, at least 94% identical, at least 95% identical, at least 96% identical, at least 98% identical, or 100% identical) to SEQ ID NO: 63.

### TGFβRII/TRβC/IL-15: Humanized Anti-Human HIVscFv/IL-1SRαSu/Anti-CD3scFv

In some embodiments, provided herein is a multi-chain chimeric polypeptide that includes: (a) a first chimeric polypeptide including: (i) a first target-binding domain that includes a soluble TGFβRII receptor (e.g., a sequence that is at least 80%, at least 90%, at least 95%, or 100% identical to SEQ ID NO: 9 or SEQ ID NO: 51); (ii) a soluble T cell receptor β-chain constant region (TRβC) domain (e.g., a sequence that is at least 80%, at least 90%, at least 95%, or 100% identical to SEQ ID NO: 1 or SEQ ID NO: 3); and (iii) a first domain of a pair of affinity domains that includes a soluble IL-15 (e.g., a sequence that is at least 80%, at least 90%, at least 95%, or 100% identical to SEQ ID NO: 13); and (b) a second chimeric polypeptide including: (i) a second target-binding domain that includes a humanized anti-human HIVscFv (e.g., a sequence that is at least 80%, at least 90%, at least 95%, or 100% identical to SEQ ID NO: 121); (ii) a second domain of a pair of affinity domains that includes the sushi domain from an alpha chain of IL-15 receptor alpha (IL-15Rα) (e.g., a sequence that is at least 80%, at least 90%, at least 95%, or 100% identical to SEQ ID NO: 11 or SEQ ID NO: 115); and (iii) an additional target-binding domain that includes an anti-CD3scFv (e.g., a sequence that is at least 80%, at least 90%, at least 95%, or 100% identical to any one of SEQ ID NO: 17, SEQ ID NO: 19, and SEQ ID NO: 133).

In some embodiments, the first chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 156)
IPPHVQKSVNNDMIVTDNNGAVKFPQLCKFCDVRFSTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPDGGGGS
IPPHVQKSVNNDMIVTDNNGAVKFPQLCKFCDVRFSTCDNQKSCMSN
CSITSICEKPQEVCVAVWRKNDENITLETVCHDPKLPYHDFILEDAA
SPKCIMKEKKKPGETFFMCSCSSDECNDNIIFSEEYNTSNPDEDLNK
VFPPEVAVFEPSEAEISHTQKATLVCLATGFFPDHVELSWWVNGKEV
HSGVSTDPQPLKEQPALNDSRYCLSSRLRVSATFWQNPRNHFRCQVQ
FYGLSENDEWTQDRAKPVTQIVSAEAWGRADNWVNVISDLKKIEDLI
QSMHIDATLYTESDVHPSCKVTAMKCFLLELQVISLESGDASIHDTV
ENLIILANNSLSSNGNVTESGCKECEELEEKNIKEFLQSFVHIVQMF
INTS.

In some embodiments, the first chimeric polypeptide further includes a signal sequence at its N-terminal end. In some embodiments the signal peptide sequence is MGVKVLFALICIAVAEA (SEQ ID NO: 137).

In some embodiments, the second chimeric polypeptide includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 190)
SYVRPLSVALGETARISCGRQALGSRAVQWYQHRPGQAPILLIYNNQ
DRPSGIPERFSGTPDINFGTRATLTISGVEAGDEADYYCHMWDSRSG
FSWSFGGATRLTVLGGGGSGGGGSGGGGSQVQLQESGPGLVKPSETL
SVTCSVSGDSMNNYYWTWIRQSPGKGLEWIGYISDRESATYNPSLNS
RVVISRDTSKNQLSLKLNSVTPADTAVYYCATARRGQRIYGVVSFGE
FFYYYSMDVWGKGTTVTVSSGGGGSITCPPPMSVEHADIWVKSYSLY
SRERYICNSGFKRKAGTSSLTECVLNKATNVAHWTTPSLKCIRDPAL
VHQRDIQMTQSPSSLSASVGDRVTITCRASQDIRNYLNWYQQKPGKA
PKLLIYYTSRLESGVPSRFSGSGSGTDYTLTISSLQPEDFATYYCQQ
GNTLPWTFGQGTKVEIKGGGGSGGGGSGGGGSEVQLVESGGGLVQPG
GSLRLSCAASGYSFTGYTMNWVRQAPGKGLEWVALINPYKGVSTYNQ
KFKDRFTISVDKSKNTAYLQMNSLRAEDTAVYYCARSGYYGDSDWYF
DVWGQGTLVTVSS

In some embodiments, the second chimeric polypeptide further includes a signal sequence at its N-terminal end. In some embodiments the signal peptide sequence is MGWSCIILFLVATATGVHS (SEQ ID NO: 139).

### Compositions/Kits

Also provided herein are compositions (e.g., pharmaceutical compositions) that include at least one of any multi-chain chimeric polypeptides, any of the cells, or any of the nucleic acids described herein. In some embodiments, the compositions include at least one of any of the multi-chain chimeric polypeptides described herein. In some embodiments, the compositions include any of the immune cells (e.g., any of the immune cells described herein, e.g., any of the immune cells produced using any of the methods described herein).

In some embodiments, the pharmaceutical compositions are formulated for different routes of administration (e.g., intravenous, subcutaneous). In some embodiments, the pharmaceutical compositions can include a pharmaceutically acceptable carrier (e.g., phosphate buffered saline).

Single or multiple administrations of pharmaceutical compositions can be given to a subject in need thereof depending on for example: the dosage and frequency as required and tolerated by the subject. The formulation should provide a sufficient quantity of active agent to effectively treat, prevent or ameliorate conditions, diseases or symptoms.

Also provided herein are kits that include any of the multi-chain chimeric polypeptides, compositions, nucleic acids, or cells (e.g., immune cells) described herein. In some embodiments, the kits can include instructions for performing any of the methods described herein. In some embodiments, the kits can include at least one dose of any of the pharmaceutical compositions described herein.

### Nucleic Acids/Vectors

Also provided herein are nucleic acids that encode any of the multi-chain chimeric polypeptides described herein. In some embodiments, a first nucleic acid can encode the first chimeric polypeptide and a second nucleic acid can encode the second chimeric polypeptide. In some embodiments, a single nucleic acid can encode both the first chimeric polypeptide and the second chimeric polypeptide.

Also provided herein are vectors that include any of the nucleic acids encoding any of the multi-chain chimeric polypeptides described herein. In some embodiments, a first vector can include a nucleic acid encoding the first chimeric polypeptide and a second vector can include a nucleic acid encoding the second chimeric polypeptide. In some embodiments, a single vector can include a first nucleic acid encoding the first chimeric polypeptide and a second nucleic acid encoding the second chimeric polypeptide.

Any of the vectors described herein can be an expression vector. For example, an expression vector can include a promoter sequence operably linked to the sequence encoding the first chimeric polypeptide and the second chimeric polypeptide.

Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, e.g., include sufficient cis-acting elements for expression; other elements for expression can be supplied by the host mammalian cell or in an in vitro expression system. Skilled practitioners will be capable of selecting suitable vectors and mammalian cells for making any of the multi-chain chimeric polypeptides described herein.

### Cells

Also provided herein are cells (e.g., any of the exemplary cells described herein or known in the art) comprising any of the nucleic acids described herein that encode any of the multi-chain chimeric polypeptides described herein (e.g., encoding both the first and second chimeric polypeptides). Also provided herein are cells (e.g., any of the exemplary cells described herein or known in the art) comprising any of the nucleic acids described herein that encode any of the first chimeric polypeptides described herein. Also provided are cells (e.g., any of the exemplary cells described herein or known in the art) comprising any of the nucleic acids described herein that encode any of the second chimeric polypeptides described herein.

Also provided herein are cells (e.g., any of the exemplary cells described herein or known in the art) that include any of the vectors described herein that encode any of the multi-chain chimeric polypeptides described herein (e.g., encoding both the first and second chimeric polypeptides). Also provided herein are cells (e.g., any of the exemplary cells described herein or known in the art) that include any of the vectors described herein that encode any of the first chimeric polypeptides described herein. Also provided herein are cells (e.g., any of the exemplary cells described herein or known in the art) that include any of the vectors described herein that encode any of the second chimeric polypeptides described herein).

In some embodiments of any of the methods described herein, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary cells and human embryonic kidney cells (e.g., HEK293 cells).

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection.

### Methods of Producing Multi-Chain Chimeric Polypeptides

Also provided herein are methods of producing any of the multi-chain chimeric polypeptides described herein that include culturing any of the cells described herein in a culture medium under conditions sufficient to result in the production of the multi-chain chimeric polypeptide; and recovering the multi-chain chimeric polypeptide from the cell and/or the culture medium.

Also provided herein are method of producing any of the multi-chain chimeric polypeptides described herein that include: culturing any of cells described herein in a first culture medium under conditions sufficient to result in the production of the first chimeric polypeptide; recovering the first chimeric polypeptide from the cell and/or the first culture medium; culturing any of the cells described herein in a second culture medium under conditions sufficient to result in the production of the second chimeric polypeptide; recovering the second chimeric polypeptide from the cell and/or the second culture medium; and combining (e.g., mixing) the recovered first chimeric polypeptide and the recovered second chimeric polypeptide to form the multi-chain chimeric polypeptide (e.g., any of the multi-chain chimeric polypeptides described herein).

The recovery of the multi-chain chimeric polypeptide, the first chimeric polypeptide, or the second chimeric polypeptide from a cell (e.g., a eukaryotic cell) can be performed using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

Methods of culturing cells are well known in the art. Cells can be maintained in vitro under conditions that favor proliferation, differentiation and growth. Briefly, cells can be cultured by contacting a cell (e.g., any cell) with a cell culture medium that includes the necessary growth factors and supplements to support cell viability and growth.

Also provided herein are multi-chain chimeric polypeptides (e.g., any of the multi-chain chimeric polypeptides described herein), first chimeric polypeptides (e.g., any of the first chimeric polypeptides), or second chimeric polypeptides (e.g., any of the second chimeric polypeptides described herein) produced by any of the methods described herein.

### Methods of Stimulating an Immune Cell

Also provided herein are methods of stimulating an immune cell (e.g., any of the exemplary immune cells described herein or known in the art) that include contacting an immune cell with an effective amount of any of the multi-chain chimeric polypeptides described herein or any of the compositions (e.g., pharmaceutical compositions) described herein. In some examples, the immune cell is contacted in vitro (e.g., in a suitable liquid culture medium under conditions sufficient to result in stimulation of the immune cell).

In some examples, the immune cell has been previously obtained from a subject (e.g., a mammal, e.g., a human). Some embodiments of these methods further include obtaining the immune cell from the subject prior to the contacting step.

In some examples, the immune cell is contacted in vivo. In such embodiments, the multi-chain chimeric polypeptide is administered to a subject (e.g., a mammal, e.g., a human) in an amount sufficient to result in stimulation of an immune cell in the subject.

In some examples of any of the methods described herein, the immune cell can be an immature thymocyte, a peripheral blood lymphocyte, a naïve T cell, a pluripotent Th cell precursor, a lymphoid progenitor cell, a Treg cell, a memory T cell, a Th17 cell, a Th22 cell, a Th9 cell, a Th2 cell, a Th1 cell, a Th3 cell, γδ T cell, an αβ T cell, a tumor-infiltrating T cell, a CD8⁺ T cell, a CD4⁺ T cell, a natural killer T cell, a mast cell, a macrophage, a neutrophil, a dendritic cell, a basophil, an eosinophil, or a natural killer cell, or a combination thereof.

In some examples, the CD8⁺ T cell can be a progenitor exhausted CD8⁺ T (Tpex) cell, an effector memory CD8⁺ T (Tem) cell, a central memory CD8⁺ T (Tcm) cell, a memory CD8⁺ stem (Tscm) cell, a virtual memory CD8⁺ T (Tvm) cell, an antigen-experienced CD8⁺ T cell, a terminally-exhausted CD8⁺ T (Tex) cell, or a senescent CD8⁺ T cell.

In some examples, the immune cell has previously been genetically-modified to express a chimeric antigen receptor or a recombinant T-cell receptor. In some examples, the immune cell (e.g., any of the immune cells described herein) has previously been genetically-modified to express a co-stimulatory molecule (e.g., CD28).

Some embodiments of these methods can further include, after the contacting step, introducing into the immune cell (e.g., any of the immune cells described herein) a nucleic acid encoding a chimeric antigen-receptor or a recombinant T-cell receptor. Some embodiments of these methods can further include, after the contacting step, introducing into the immune cell (e.g., any of the immune cells described herein) a nucleic acid encoding a co-stimulatory molecule (e.g., CD28).

Some embodiments of these methods can further include administering a therapeutically effective amount of the immune cell to a subject in need thereof (e.g., any of the exemplary subjects described herein).

In some examples, the subject can be a subject identified or diagnosed as having an age-related disease or condition. Non-limiting examples of age-related diseases or disorders include: Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, bronchopulmonary dysplasia, and renal dysfunction.

In some examples, the subject can be a subject that has been identified or diagnosed as having a cancer. Non-limiting examples of cancers include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma.

In some examples, the subject can be a subject that has been diagnosed or identified as having an infectious disease. Non-limiting examples of infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, or influenza virus.

Activation of an immune cell can be determined using methods known in the art. For example, activation of an immune cell can be determined by detecting the levels of cytokines and chemokines that are secreted or cytotoxicity granules and regulatory molecules that are upregulated upon activation of an immune cell. Non-limiting examples of cytokines, chemokines, cytotoxicity granules, and regulatory molecules that are secreted or upregulated upon activation of an immune cell include: IL-2, IFN-γ, IL-1, IL-4, IL-5, IL-6, IL-7, IL-9, IL-10, IL-12, IL-13, IL-15, IL-17, IL-18, IL-22, IL-33, leukotriene B4, CCL5, TNFα, granzymes, perforin, TGFβ, STAT3, STAT4, STAT5, RORγT, FOXP3, STAT6, and GATA3. The detection of these cytokines, chemokines, cytotoxicity granules, or regulatory molecules can be performed using an immunoassay (e.g., an enzyme-linked immunosorbent assay) and quantitative PCR. For example, activation of an immune cell can result in an increase of about 1% to about 800% (e.g., about 1% to about 750%, about 1% to about 700%, about 1% to about 650%, about 1% to about 600%, about 1% to about 550%, about 1% to about 500%, about 1% to about 450%, about 1% to about 400%, about 1% to about 350%, about 1% to about 300%, about 1% to about 280%, about 1% to about 260%, about 1% to about 240%, about 1% to about 220%, about 1% to about 200%, about 1% to about 180%, about 1% to about 160%, about 1% to about 140%, about 1% to about 120%, about 1% to about 100%, about 1% to about 90%, about 1% to about 80%, about 1% to about 70%, about 1% to about 60%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 800%, about 5% to about 750%, about 5% to about 700%, about 5% to about 650%, about 5% to about 600%, about 5% to about 550%, about 5% to about 500%, about 5% to about 450%, about 5% to about 400%, about 5% to about 350%, about 5% to about 300%, about 5% to about 280%, about 5% to about 260%, about 5% to about 240%, about 5% to about 220%, about 5% to about 200%, about 5% to about 180%, about 5% to about 160%, about 5% to about 140%, about 5% to about 120%, about 5% to about 100%, about 5% to about 90%, about 5% to about 80%, about 5% to about 70%, about 5% to about 60%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 800%, about 10% to about 750%, about 10% to about 700%, about 10% to about 650%, about 10% to about 600%, about 10% to about 550%, about 10% to about 500%, about 10% to about 450%, about 10% to about 400%, about 10% to about 350%, about 10% to about 300%, about 10% to about 280%, about 10% to about 260%, about 10% to about 240%, about 10% to about 220%, about 10% to about 200%, about 10% to about 180%, about 10% to about 160%, about 10% to about 140%, about 10% to about 120%, about 10% to about 100%, about 10% to about 90%, about 10% to about 80%, about 10% to about 70%, about 10% to about 60%, about 10% to about 50%, about 10% to about 45%, about 10% to about 40%, about 10% to about 35%, about 10% to about 30%, about 10% to about 25%, about 10% to about 20%, about 10% to about 15%, about 15% to about 800%, about 15% to about 750%, about 15% to about 700%, about 15% to about 650%, about 15% to about 600%, about 15% to about 550%, about 15% to about 500%, about 15% to about 450%, about 15% to about 400%, about 15% to about 350%, about 15% to about 300%, about 15% to about 280%, about 15% to about 260%, about 15% to about 240%, about 15% to about 220%, about 15% to about 200%, about 15% to about 180%, about 15% to about 160%, about 15% to about 140%, about 15% to about 120%, about 15% to about 100%, about 15% to about 90%, about 15% to about 80%, about 15% to about 70%, about 15% to about 60%, about 15% to about 50%, about 15% to about 45%, about 15% to about 40%, about 15% to about 35%, about 15% to about 30%, about 15% to about 25%, about 15% to about 20%, about 20% to about 800%, about 20% to about 750%, about 20% to about 700%, about 20% to about 650%, about 20% to about 600%, about 20% to about 550%, about 20% to about 500%, about 20% to about 450%, about 20% to about 400%, about 20% to about 350%, about 20% to about 300%, about 20% to about 280%, about 20% to about 260%, about 20% to about 240%, about 20% to about 220%, about 20% to about 200%, about 20% to about 180%, about 20% to about 160%, about 20% to about 140%, about 20% to about 120%, about 20% to about 100%, about 20% to about 90%, about 20% to about 80%, about 20% to about 70%, about 20% to about 60%, about 20% to about 50%, about 20% to about 45%, about 20% to about 40%, about 20% to about 35%, about 20% to about 30%, about 20% to about 25%, about 25% to about 800%, about 25% to about 750%, about 25% to about 700%, about 25% to about 650%, about 25% to about 600%, about 25% to about 550%, about 25% to about 500%, about 25% to about 450%, about 25% to about 400%, about 25% to about 350%, about 25% to about 300%, about 25% to about 280%, about 25% to about 260%, about 25% to about 240%, about 25% to about 220%, about 25% to about 200%, about 25% to about 180%, about 25% to about 160%, about 25% to about 140%, about 25% to about 120%, about 25% to about 100%, about 25% to about 90%, about 25% to about 80%, about 25% to about 70%, about 25% to about 60%, about 25% to about 50%, about 25% to about 45%, about 25% to about 40%, about 25% to about 35%, about 35% to about 800%, about 35% to about 750%, about 35% to about 700%, about 35% to about 650%, about 35% to about 600%, about 35% to about 550%, about 35% to about 500%, about 35% to about 450%, about 35% to about 400%, about 35% to about 350%, about 35% to about 300%, about 35% to about 280%, about 35% to about 260%, about 35% to about 240%, about 35% to about 220%, about 35% to about 200%, about 35% to about 180%, about 35% to about 160%, about 35% to about 140%, about 35% to about 120%, about 35% to about 100%, about 35% to about 90%, about 35% to about 80%, about 35% to about 70%, about 35% to about 60%, about 35% to about 50%, about 35% to about 45%, about 35% to about 40%, about 40% to about 800%, about 40% to about 750%, about 40% to about 700%, about 40% to about 650%, about 40% to about 600%, about 40% to about 550%, about 40% to about 500%, about 40% to about 450%, about 40% to about 400%, about 40% to about 350%, about 40% to about 300%, about 40% to about 280%, about 40% to about 260%, about 40% to about 240%, about 40% to about 220%, about 40% to about 200%, about 40% to about 180%, about 40% to about 160%, about 40% to about 140%, about 40% to about 120%, about 40% to about 100%, about 40% to about 90%, about 40% to about 80%, about 40% to about 70%, about 40% to about 60%, about 40% to about 50%, about 40% to about 45%, about 45% to about 800%, about 45% to about 750%, about 45% to about 700%, about 45% to about 650%, about 45% to about 600%, about 45% to about 550%, about 45% to about 500%, about 45% to about 450%, about 45% to about 400%, about 45% to about 350%, about 45% to about 300%, about 45% to about 280%, about 45% to about 260%, about 45% to about 240%, about 45% to about 220%, about 45% to about 200%, about 45% to about 180%, about 45% to about 160%, about 45% to about 140%, about 45% to about 120%, about 45% to about 100%, about 45% to about 90%, about 45% to about 80%, about 45% to about 70%, about 45% to about 60%, about 45% to about 50%, about 50% to about 800%, about 50% to about 750%, about 50% to about 700%, about 50% to about 650%, about 50% to about 600%, about 50% to about 550%, about 50% to about 500%, about 50% to about 450%, about 50% to about 400%, about 50% to about 350%, about 50% to about 300%, about 50% to about 280%, about 50% to about 260%, about 50% to about 240%, about 50% to about 220%, about 50% to about 200%, about 50% to about 180%, about 50% to about 160%, about 50% to about 140%, about 50% to about 120%, about 50% to about 100%, about 50% to about 90%, about 50% to about 80%, about 50% to about 70%, about 50% to about 60%, about 60% to about 800%, about 60% to about 750%, about 60% to about 700%, about 60% to about 650%, about 60% to about 600%, about 60% to about 550%, about 60% to about 500%, about 60% to about 450%, about 60% to about 400%, about 60% to about 350%, about 60% to about 300%, about 60% to about 280%, about 60% to about 260%, about 60% to about 240%, about 60% to about 220%, about 60% to about 200%, about 60% to about 180%, about 60% to about 160%, about 60% to about 140%, about 60% to about 120%, about 60% to about 100%, about 60% to about 90%, about 60% to about 80%, about 60% to about 70%, about 70% to about 800%, about 70% to about 750%, about 70% to about 700%, about 70% to about 650%, about 70% to about 600%, about 70% to about 550%, about 70% to about 500%, about 70% to about 450%, about 70% to about 400%, about 70% to about 350%, about 70% to about 300%, about 70% to about 280%, about 70% to about 260%, about 70% to about 240%, about 70% to about 220%, about 70% to about 200%, about 70% to about 180%, about 70% to about 160%, about 70% to about 140%, about 70% to about 120%, about 70% to about 100%, about 70% to about 90%, about 70% to about 80%, about 80% to about 800%, about 80% to about 750%, about 80% to about 700%, about 80% to about 650%, about 80% to about 600%, about 80% to about 550%, about 80% to about 500%, about 80% to about 450%, about 80% to about 400%, about 80% to about 350%, about 80% to about 300%, about 80% to about 280%, about 80% to about 260%, about 80% to about 240%, about 80% to about 220%, about 80% to about 200%, about 80% to about 180%, about 80% to about 160%, about 80% to about 140%, about 80% to about 120%, about 80% to about 100%, about 80% to about 90%, about 90% to about 800%, about 90% to about 750%, about 90% to about 700%, about 90% to about 650%, about 90% to about 600%, about 90% to about 550%, about 90% to about 500%, about 90% to about 450%, about 90% to about 400%, about 90% to about 350%, about 90% to about 300%, about 90% to about 280%, about 90% to about 260%, about 90% to about 240%, about 90% to about 220%, about 90% to about 200%, about 90% to about 180%, about 90% to about 160%, about 90% to about 140%, about 90% to about 120%, about 90% to about 100%, about 100% to about 800%, about 100% to about 750%, about 100% to about 700%, about 100% to about 650%, about 100% to about 600%, about 100% to about 550%, about 100% to about 500%, about 100% to about 450%, about 100% to about 400%, about 100% to about 350%, about 100% to about 300%, about 100% to about 280%, about 100% to about 260%, about 100% to about 240%, about 100% to about 220%, about 100% to about 200%, about 100% to about 180%, about 100% to about 160%, about 100% to about 140%, about 100% to about 120%, about 120% to about 800%, about 120% to about 750%, about 120% to about 700%, about 120% to about 650%, about 120% to about 600%, about 120% to about 550%, about 120% to about 500%, about 120% to about 450%, about 120% to about 400%, about 120% to about 350%, about 120% to about 300%, about 120% to about 280%, about 120% to about 260%, about 120% to about 240%, about 120% to about 220%, about 120% to about 200%, about 120% to about 180%, about 120% to about 160%, about 120% to about 140%, about 140% to about 800%, about 140% to about 750%, about 140% to about 700%, about 140% to about 650%, about 140% to about 600%, about 140% to about 550%, about 140% to about 500%, about 140% to about 450%, about 140% to about 400%, about 140% to about 350%, about 140% to about 300%, about 140% to about 280%, about 140% to about 260%, about 140% to about 240%, about 140% to about 220%, about 140% to about 200%, about 140% to about 180%, about 140% to about 160%, about 160% to about 800%, about 160% to about 750%, about 160% to about 700%, about 160% to about 650%, about 160% to about 600%, about 160% to about 550%, about 160% to about 500%, about 160% to about 450%, about 160% to about 400%, about 160% to about 350%, about 160% to about 300%, about 160% to about 280%, about 160% to about 260%, about 160% to about 240%, about 160% to about 220%, about 160% to about 200%, about 160% to about 180%, about 180% to about 800%, about 180% to about 750%, about 180% to about 700%, about 180% to about 650%, about 180% to about 600%, about 180% to about 550%, about 180% to about 500%, about 180% to about 450%, about 180% to about 400%, about 180% to about 350%, about 180% to about 300%, about 180% to about 280%, about 180% to about 260%, about 180% to about 240%, about 180% to about 220%, about 180% to about 200%, about 200% to about 800%, about 200% to about 750%, about 200% to about 700%, about 200% to about 650%, about 200% to about 600%, about 200% to about 550%, about 200% to about 500%, about 200% to about 450%, about 200% to about 400%, about 200% to about 350%, about 200% to about 300%, about 200% to about 280%, about 200% to about 260%, about 200% to about 240%, about 200% to about 220%, about 220% to about 800%, about 220% to about 750%, about 220% to about 700%, about 220% to about 650%, about 220% to about 600%, about 220% to about 550%, about 220% to about 500%, about 220% to about 450%, about 220% to about 400%, about 220% to about 350%, about 220% to about 300%, about 220% to about 280%, about 220% to about 260%, about 220% to about 240%, about 240% to about 800%, about 240% to about 750%, about 240% to about 700%, about 240% to about 650%, about 240% to about 600%, about 240% to about 550%, about 240% to about 500%, about 240% to about 450%, about 240% to about 400%, about 240% to about 350%, about 240% to about 300%, about 240% to about 280%, about 240% to about 260%, about 260% to about 800%, about 260% to about 750%, about 260% to about 700%, about 260% to about 650%, about 260% to about 600%, about 260% to about 550%, about 260% to about 500%, about 260% to about 450%, about 260% to about 400%, about 260% to about 350%, about 260% to about 300%, about 260% to about 280%, about 280% to about 800%, about 280% to about 750%, about 280% to about 700%, about 280% to about 650%, about 280% to about 600%, about 280% to about 550%, about 280% to about 500%, about 280% to about 450%, about 280% to about 400%, about 280% to about 350%, about 280% to about 300%, about 300% to about 800%, about 300% to about 750%, about 300% to about 700%, about 300% to about 650%, about 300% to about 600%, about 300% to about 550%, about 300% to about 500%, about 300% to about 450%, about 300% to about 400%, about 300% to about 350%, about 350% to about 800%, about 350% to about 750%, about 350% to about 700%, about 350% to about 650%, about 350% to about 600%, about 350% to about 550%, about 350% to about 500%, about 350% to about 450%, about 350% to about 400%, about 400% to about 800%, about 400% to about 750%, about 400% to about 700%, about 400% to about 650%, about 400% to about 600%, about 400% to about 550%, about 400% to about 500%, about 400% to about 450%, about 450% to about 800%, about 450% to about 750%, about 450% to about 700%, about 450% to about 650%, about 450% to about 600%, about 450% to about 550%, about 450% to about 500%, about 500% to about 800%, about 500% to about 750%, about 500% to about 700%, about 500% to about 650%, about 500% to about 600%, about 500% to about 550%, about 550% to about 800%, about 550% to about 750%, about 550% to about 700%, about 550% to about 650%, about 550% to about 600%, about 600% to about 800%, about 600% to about 750%, about 600% to about 700%, about 600% to about 650%, about 650% to about 800%, about 650% to about 750%, about 650% to about 700%, about 700% to about 800%, about 700% to about 750%, or about 750% to about 800%) of one or more of any of the cytokines or chemokines or cytotoxicity granules or regulatory molecules described herein (e.g., one or more of any of IL-2, IFN-γ, IL-1, IL-4, IL-5, IL-6, IL-7, IL-9, IL-10, IL-12, IL-13, IL-15, IL-17, IL-18, IL-22, IL-33, leukotriene B4, CCL5, TNFα, granzymes, perforin, TGFβ, STAT3, STAT4, STAT5, RORγT, FOXP3, and GATA3) (e.g., as compared to the level of the one or more cytokines, chemokines, cytotoxicity granules, and regulatory molecules in a control not contacted with any of the multi-chain chimeric polypeptides described herein).

### Methods of Inducing or Increasing Proliferation of an Immune Cell

Also provided herein are methods of inducing or increasing proliferation of an immune cell (e.g., any of the exemplary immune cells described herein or known in the art) that include contacting an immune cell with an effective amount of any of the multi-chain chimeric polypeptides described herein or any of the compositions (e.g., pharmaceutical compositions) described herein. In some examples, the immune cell is contacted in vitro (e.g., in a suitable liquid culture medium under conditions sufficient to result in stimulation of the immune cell).

In some examples, the immune cell has been previously obtained from a subject (e.g., a mammal, e.g., a human). Some embodiments of these methods further include obtaining the immune cell from the subject prior to the contacting step.

In some examples, the immune cell is contacted in vivo. In such embodiments, the multi-chain chimeric polypeptide is administered to a subject (e.g., a mammal, e.g., a human) in an amount sufficient to result in stimulation of an immune cell in the subject.

In some examples of any of the methods described herein, the immune cell can be an immature thymocyte, a peripheral blood lymphocyte, a naïve T cell, a pluripotent Th cell precursor, a lymphoid progenitor cell, a Treg cell, a memory T cell, a Th17 cell, a Th22 cell, a Th9 cell, a Th2 cell, a Th1 cell, a Th3 cell, γδ T cell, an αβ T cell, a tumor-infiltrating T cell, a CD8⁺ T cell, a CD4⁺ T cell, a natural killer T cell, a mast cell, a macrophage, a neutrophil, a dendritic cell, a basophil, an eosinophil, or a natural killer cell, or a combination thereof.

In some examples, the CD8m T cell can be a progenitor exhausted CD8⁺ T (Tpex) cell, an effector memory CD8⁺ T (Tem) cell, a central memory CD8⁺ T (Tcm) cell, a memory CD8⁺ stem (Tscm) cell, a virtual memory CD8⁺ T (Tvm) cell, an antigen-experienced CD8⁺ T cell, a terminally-exhausted CD8⁺ T (Tex) cell, or a senescent CD8+ T cell.

In some examples, the immune cell has previously been genetically-modified to express a chimeric antigen receptor or a recombinant T-cell receptor. In some examples, the immune cell (e.g., any of the immune cells described herein) has previously been genetically-modified to express a co-stimulatory molecule (e.g., CD28).

Some embodiments of these methods can further include, after the contacting step, introducing into the immune cell (e.g., any of the immune cells described herein) a nucleic acid encoding a chimeric antigen-receptor or a recombinant T-cell receptor. Some embodiments of these methods can further include, after the contacting step, introducing into the immune cell (e.g., any of the immune cells described herein) a nucleic acid encoding a co-stimulatory molecule (e.g., CD28).

Some embodiments of these methods can further include administering a therapeutically effective amount of the immune cell to a subject in need thereof (e.g., any of the exemplary subjects described herein).

In some examples, the subject can be a subject identified or diagnosed as having an age-related disease or condition. Non-limiting examples of age-related diseases or disorders include: Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, bronchopulmonary dysplasia, and renal dysfunction.

In some examples, the subject can be a subject that has been identified or diagnosed as having a cancer. Non-limiting examples of cancers include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma.

In some examples, the subject can be a subject that has been diagnosed or identified as having an infectious disease. Non-limiting examples of infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, or influenza virus.

Detection of the proliferation of an immune cell can be performed using methods known in the art, e.g., cytometry (e.g., fluorescence-assisted flow cytometry), microscopy, and immunofluorescence microscopy, e.g., by comparing the rate of increase in the concentration of the immune cell in a sample not contacted with a multi-chain chimeric polypeptide to the rate of increase in the concentration of the immune cell in a similar sample contacted with any of the multi-chain chimeric polypeptides described herein).

In other examples, the proliferation of an immune cell can be indirectly detected by detecting an increase in the level of one or more cytokines or chemokines or cytotoxicity granules or regulatory molecules secreted or upregulated by proliferating immune cells (e.g., one or more of IL-2, IFN-γ, IL-1, IL-4, IL-5, IL-6, IL-7, IL-9, IL-10, IL-12, IL-13, IL-15, IL-17, IL-18, IL-22, IL-33, leukotriene B4, CCL5, TNFα, granzymes, perforin, TGFβ, STAT3, STAT4, STAT5, RORγT, FOXP3, and GATA3) (e.g., as compared to the level of the one or more cytokines, chemokines, cytotoxicity granules, and regulatory molecules in a control not contacted with any of the multi-chain chimeric polypeptides described herein).

In some embodiments, the methods provided herein can result in an increase (e.g., about 1% to about 800% increase, or any of the subranges of this range described herein) in the rate of increase in the concentration of the immune cell in a sample contacted with any of the multi-chain chimeric polypeptides described herein as compared to the rate of increase in a similar control sample not contacted with any of the multi-chain chimeric polypeptides described herein.

### Methods of Inducing Differentiation of an Immune Cell

Also provided herein are method of inducing differentiation of an immune cell (e.g., any of the exemplary immune cells described herein or known in the art) into a memory or memory-like immune cell that include contacting an immune cell with an effective amount of any of the multi-chain chimeric polypeptides described herein or any of the compositions (e.g., pharmaceutical compositions) described herein. In some examples, the immune cell is contacted in vitro (e.g., in a suitable liquid culture medium under conditions sufficient to result in stimulation of the immune cell).

In some examples, the immune cell has been previously obtained from a subject (e.g., a mammal, e.g., a human). Some embodiments of these methods further include obtaining the immune cell from the subject prior to the contacting step.

In some examples, the immune cell is contacted in vivo. In such embodiments, the multi-chain chimeric polypeptide is administered to a subject (e.g., a mammal, e.g., a human) in an amount sufficient to result in stimulation of an immune cell in the subject.

In some examples of any of the methods described herein, the immune cell can be an immature thymocyte, a peripheral blood lymphocyte, a naïve T cell, a pluripotent Th cell precursor, a lymphoid progenitor cell, a Treg cell, a Th17 cell, a Th22 cell, a Th9 cell, a Th2 cell, a Th1 cell, a Th3 cell, γδ T cell, an αβ T cell, a tumor-infiltrating T cell, a CD8⁺ T cell, a CD4⁺ T cell, a natural killer T cell, a mast cell, a macrophage, a neutrophil, a dendritic cell, a basophil, an eosinophil, or a natural killer cell, or a combination thereof.

In some examples, the CD8⁺ T cell can be a progenitor exhausted CD8⁺ T (Tpex) cell, an effector memory CD8⁺ T (Tem) cell, a central memory CD8⁺ T (Tcm) cell, a memory CD8⁺stem (Tscm) cell, a virtual memory CD8⁺ T (Tvm) cell, an antigen-experienced CD8⁺ T cell, a terminally-exhausted CD8⁺ T (Tex) cell, or a senescent CD8⁺ T cell.

In some examples, the immune cell has previously been genetically-modified to express a chimeric antigen receptor or a recombinant T-cell receptor. In some examples, the immune cell (e.g., any of the immune cells described herein) has previously been genetically-modified to express a co-stimulatory molecule (e.g., CD28).

In some examples, an effective amount of any of the multi-chain chimeric polypeptides described herein or any of the compositions (e.g., pharmaceutical compositions) described herein is combined with an anti-hTRβC IgG1 antibody to create a memory or memory like immune cell.

Some embodiments of these methods can further include, after the contacting step, introducing into the immune cell (e.g., any of the immune cells described herein) a nucleic acid encoding a chimeric antigen-receptor or a recombinant T-cell receptor. Some embodiments of these methods can further include, after the contacting step, introducing into the immune cell (e.g., any of the immune cells described herein) a nucleic acid encoding a co-stimulatory molecule (e.g., CD28).

Some embodiments of these methods can further include administering a therapeutically effective amount of the immune cell to a subject in need thereof (e.g., any of the exemplary subjects described herein).

In some examples, the subject can be a subject identified or diagnosed as having an age-related disease or condition. Non-limiting examples of age-related diseases or disorders include: Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, bronchopulmonary dysplasia, and renal dysfunction.

In some examples, the subject can be a subject that has been identified or diagnosed as having a cancer. Non-limiting examples of cancers include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma.

In some examples, the subject can be a subject that has been diagnosed or identified as having an infectious disease. Non-limiting examples of infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, or influenza virus.

In some examples, the immune cell is a NK cell, and the detection of a memory NK cell can include, e.g., the detection of the level of one or more of IL-12, IL-18, IL-33, CD25, CD69, CD62L, STAT4, Zbtb32, DNAM-1, NKp30, NKp44, NKp46, BIM, Noxa, SOCS1, BNIP3, BNIP3L, IFN-7, CXCL16, CXCR6, NKG2D, TRAIL, CD49, Ly49D, CD49b, and Ly79H. A description of NK memory cells and methods of detecting the same is described in O'Sullivan et al., Immunity 43:634-645, 2015.

In some examples, the immune cell is a T cell, and the detection of memory T cells can include, e.g., the detection of the level of expression of one or more of CD45RO, CCR7, L-selectin (CD62L), CD44, CD45RA, integrin αeβ7, CD43, CD27, CD28, CD127 (IL-7Rα), CD69, CD95, IL-2Rβ, KLRG-1, LY108, CCR7, CXCR3, CXCR5, CX3CR1, PD-1, TIM3, TIGIT, LAG-3, CTLA-4, and LFA-1. In some examples, the immune cell is a B cell and the detection of memory B cells can include, e.g., the detection of the level of expression of CD27. Other types and markers of memory or memory-like immune cells are known in the art.

### Methods of Treatment

Also provided herein are methods of treating a subject in need thereof (e.g., any of the exemplary subjects described herein or known in the art) that include administering to the subject a therapeutically effective amount of any of the multi-chain chimeric polypeptides described herein or any of the compositions (e.g., pharmaceutical compositions) described herein.

In some embodiments of these methods, the subject has been identified or diagnosed as having a cancer. Non-limiting examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma. In some embodiments, these methods can result in a reduction in the number, severity, or frequency of one or more symptoms of the cancer in the subject (e.g., as compared to the number, severity, or frequency of the one or more symptoms of the cancer in the subject prior to treatment). In some embodiments, these methods can result in a reduction (e.g., about 1% reduction to about 99% reduction, about 1% reduction to about 95% reduction, about 1% reduction to about 90% reduction, about 1% reduction to about 85% reduction, about 1% reduction to about 80% reduction, about 1% reduction to about 75% reduction, about 1% reduction to about 70% reduction, about 1% reduction to about 65% reduction, about 1% reduction to about 60% reduction, about 1% reduction to about 55% reduction, about 1% reduction to about 50% reduction, about 1% reduction to about 45% reduction, about 1% reduction to about 40% reduction, about 1% reduction to about 35% reduction, about 1% reduction to about 30% reduction, about 1% reduction to about 25% reduction, about 1% reduction to about 20% reduction, about 1% reduction to about 15% reduction, about 1% reduction to about 10% reduction, about 1% reduction to about 5% reduction, about 5% reduction to about 99% reduction, about 5% reduction to about 95% reduction, about 5% reduction to about 90% reduction, about 5% reduction to about 85% reduction, about 5% reduction to about 80% reduction, about 5% reduction to about 75% reduction, about 5% reduction to about 70% reduction, about 5% reduction to about 65% reduction, about 5% reduction to about 60% reduction, about 5% reduction to about 55% reduction, about 5% reduction to about 50% reduction, about 5% reduction to about 45% reduction, about 5% reduction to about 40% reduction, about 5% reduction to about 35% reduction, about 5% reduction to about 30% reduction, about 5% reduction to about 25% reduction, about 5% reduction to about 20% reduction, about 5% reduction to about 15% reduction, about 5% reduction to about 10% reduction, about 10% reduction to about 99% reduction, about 10% reduction to about 95% reduction, about 10% reduction to about 90% reduction, about 10% reduction to about 85% reduction, about 10% reduction to about 80% reduction, about 10% reduction to about 75% reduction, about 10% reduction to about 70% reduction, about 10% reduction to about 65% reduction, about 10% reduction to about 60% reduction, about 10% reduction to about 55% reduction, about 10% reduction to about 50% reduction, about 10% reduction to about 45% reduction, about 10% reduction to about 40% reduction, about 10% reduction to about 35% reduction, about 10% reduction to about 30% reduction, about 10% reduction to about 25% reduction, about 10% reduction to about 20% reduction, about 10% reduction to about 15% reduction, about 15% reduction to about 99% reduction, about 15% reduction to about 95% reduction, about 15% reduction to about 90% reduction, about 15% reduction to about 85% reduction, about 15% reduction to about 80% reduction, about 15% reduction to about 75% reduction, about 15% reduction to about 70% reduction, about 15% reduction to about 65% reduction, about 15% reduction to about 60% reduction, about 15% reduction to about 55% reduction, about 15% reduction to about 50% reduction, about 15% reduction to about 45% reduction, about 15% reduction to about 40% reduction, about 15% reduction to about 35% reduction, about 15% reduction to about 30% reduction, about 15% reduction to about 25% reduction, about 15% reduction to about 20% reduction, about 20% reduction to about 99% reduction, about 20% reduction to about 95% reduction, about 20% reduction to about 90% reduction, about 20% reduction to about 85% reduction, about 20% reduction to about 80% reduction, about 20% reduction to about 75% reduction, about 20% reduction to about 70% reduction, about 20% reduction to about 65% reduction, about 20% reduction to about 60% reduction, about 20% reduction to about 55% reduction, about 20% reduction to about 50% reduction, about 20% reduction to about 45% reduction, about 20% reduction to about 40% reduction, about 20% reduction to about 35% reduction, about 20% reduction to about 30% reduction, about 20% reduction to about 25% reduction, about 25% reduction to about 99% reduction, about 25% reduction to about 95% reduction, about 25% reduction to about 90% reduction, about 25% reduction to about 85% reduction, about 25% reduction to about 80% reduction, about 25% reduction to about 75% reduction, about 25% reduction to about 70% reduction, about 25% reduction to about 65% reduction, about 25% reduction to about 60% reduction, about 25% reduction to about 55% reduction, about 25% reduction to about 50% reduction, about 25% reduction to about 45% reduction, about 25% reduction to about 40% reduction, about 25% reduction to about 35% reduction, about 25% reduction to about 30% reduction, about 30% reduction to about 99% reduction, about 30% reduction to about 95% reduction, about 30% reduction to about 90% reduction, about 30% reduction to about 85% reduction, about 30% reduction to about 80% reduction, about 30% reduction to about 75% reduction, about 30% reduction to about 70% reduction, about 30% reduction to about 65% reduction, about 30% reduction to about 60% reduction, about 30% reduction to about 55% reduction, about 30% reduction to about 50% reduction, about 30% reduction to about 45% reduction, about 30% reduction to about 40% reduction, about 30% reduction to about 35% reduction, about 35% reduction to about 99% reduction, about 35% reduction to about 95% reduction, about 35% reduction to about 90% reduction, about 35% reduction to about 85% reduction, about 35% reduction to about 80% reduction, about 35% reduction to about 75% reduction, about 35% reduction to about 70% reduction, about 35% reduction to about 65% reduction, about 35% reduction to about 60% reduction, about 35% reduction to about 55% reduction, about 35% reduction to about 50% reduction, about 35% reduction to about 45% reduction, about 35% reduction to about 40% reduction, about 40% reduction to about 99% reduction, about 40% reduction to about 95% reduction, about 40% reduction to about 90% reduction, about 40% reduction to about 85% reduction, about 40% reduction to about 80% reduction, about 40% reduction to about 75% reduction, about 40% reduction to about 70% reduction, about 40% reduction to about 65% reduction, about 40% reduction to about 60% reduction, about 40% reduction to about 55% reduction, about 40% reduction to about 50% reduction, about 40% reduction to about 45% reduction, about 45% reduction to about 99% reduction, about 45% reduction to about 95% reduction, about 45% reduction to about 90% reduction, about 45% reduction to about 85% reduction, about 45% reduction to about 80% reduction, about 45% reduction to about 75% reduction, about 45% reduction to about 70% reduction, about 45% reduction to about 65% reduction, about 45% reduction to about 60% reduction, about 45% reduction to about 55% reduction, about 45% reduction to about 50% reduction, about 50% reduction to about 99% reduction, about 50% reduction to about 95% reduction, about 50% reduction to about 90% reduction, about 50% reduction to about 85% reduction, about 50% reduction to about 80% reduction, about 50% reduction to about 75% reduction, about 50% reduction to about 70% reduction, about 50% reduction to about 65% reduction, about 50% reduction to about 60% reduction, about 50% reduction to about 55% reduction, about 55% reduction to about 99% reduction, about 55% reduction to about 95% reduction, about 55% reduction to about 90% reduction, about 55% reduction to about 85% reduction, about 55% reduction to about 80% reduction, about 55% reduction to about 75% reduction, about 55% reduction to about 70% reduction, about 55% reduction to about 65% reduction, about 55% reduction to about 60% reduction, about 60% reduction to about 99% reduction, about 60% reduction to about 95% reduction, about 60% reduction to about 90% reduction, about 60% reduction to about 85% reduction, about 60% reduction to about 80% reduction, about 60% reduction to about 75% reduction, about 60% reduction to about 70% reduction, about 60% reduction to about 65% reduction, about 65% reduction to about 99% reduction, about 65% reduction to about 95% reduction, about 65% reduction to about 90% reduction, about 65% reduction to about 85% reduction, about 65% reduction to about 80% reduction, about 65% reduction to about 75% reduction, about 65% reduction to about 70% reduction, about 70% reduction to about 99% reduction, about 70% reduction to about 95% reduction, about 70% reduction to about 90% reduction, about 70% reduction to about 85% reduction, about 70% reduction to about 80% reduction, about 70% reduction to about 75% reduction, about 75% reduction to about 99% reduction, about 75% reduction to about 95% reduction, about 75% reduction to about 90% reduction, about 75% reduction to about 85% reduction, about 75% reduction to about 80% reduction, about 80% reduction to about 99% reduction, about 80% reduction to about 95% reduction, about 80% reduction to about 90% reduction, about 80% reduction to about 85% reduction, about 85% reduction to about 99% reduction, about 85% reduction to about 95% reduction, about 85% reduction to about 90% reduction, about 90% reduction to about 99% reduction, about 90% reduction to about 95% reduction, or about 95% reduction to about 99% reduction) in the volume of one or more solid tumors in the subject (e.g., as compared to the volume of the one or more solid tumors prior to treatment or at the start of treatment). In some embodiments, the these methods can reduce (e.g., about 1% reduction to about 99% reduction, or any of the subranges of this range described herein) the risk of developing a metastasis or developing one or more additional metastasis in a subject (e.g., as compared to the risk of developing a metastasis or developing one or more additional metastasis in a subject prior to treatment or in a similar subject or a population of subjects administered a different treatment).

In some examples of these methods, the subject has been identified or diagnosed as having an aging-related disease or condition. Non-limiting examples of aging-related diseases and conditions include Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, and renal dysfunction. In some examples, these methods can result in a reduction in the number, severity, or frequency of one or more symptoms of the aging-related disease or condition in the subject (e.g., as compared to the number, severity, or frequency of the one or more symptoms of the aging-related disease or condition in the subject prior to treatment). In some examples, the methods can result in a decrease (e.g., about 1% decrease to about 99% decrease, an about 1% decrease to about 95% decrease, about 1% decrease to about 90% decrease, about 1% decrease to about 85% decrease, about 1% decrease to about 80% decrease, about 1% decrease to about 75% decrease, about 1% to about 70% decrease, about 1% decrease to about 65% decrease, about 1% decrease to about 60% decrease, about 1% decrease to about 55% decrease, about 1% decrease to about 50% decrease, about 1% decrease to about 45% decrease, about 1% decrease to about 40% decrease, about 1% decrease to about 35% decrease, about 1% decrease to about 30% decrease, about 1% decrease to about 25% decrease, about 1% decrease to about 20% decrease, about 1% decrease to about 15% decrease, about 1% decrease to about 10% decrease, about 1% decrease to about 5% decrease, about 5% decrease to about 99% decrease, an about 5% decrease to about 95% decrease, about 5% decrease to about 90% decrease, about 5% decrease to about 85% decrease, about 5% decrease to about 80% decrease, about 5% decrease to about 75% decrease, about 5% to about 70% decrease, about 5% decrease to about 65% decrease, about 5% decrease to about 60% decrease, about 5% decrease to about 55% decrease, about 5% decrease to about 50% decrease, about 5% decrease to about 45% decrease, about 5% decrease to about 40% decrease, about 5% decrease to about 35% decrease, about 5% decrease to about 30% decrease, about 5% decrease to about 25% decrease, about 5% decrease to about 20% decrease, about 5% decrease to about 15% decrease, about 5% decrease to about 10% decrease, about 10% decrease to about 99% decrease, an about 10% decrease to about 95% decrease, about 10% decrease to about 90% decrease, about 10% decrease to about 85% decrease, about 10% decrease to about 80% decrease, about 10% decrease to about 75% decrease, about 10% to about 70% decrease, about 10% decrease to about 65% decrease, about 10% decrease to about 60% decrease, about 10% decrease to about 55% decrease, about 10% decrease to about 50% decrease, about 10% decrease to about 45% decrease, about 10% decrease to about 40% decrease, about 10% decrease to about 35% decrease, about 10% decrease to about 30% decrease, about 10% decrease to about 25% decrease, about 10% decrease to about 20% decrease, about 10% decrease to about 15% decrease, about 15% decrease to about 99% decrease, an about 15% decrease to about 95% decrease, about 15% decrease to about 90% decrease, about 15% decrease to about 85% decrease, about 15% decrease to about 80% decrease, about 15% decrease to about 75% decrease, about 15% to about 70% decrease, about 15% decrease to about 65% decrease, about 15% decrease to about 60% decrease, about 15% decrease to about 55% decrease, about 15% decrease to about 50% decrease, about 15% decrease to about 45% decrease, about 15% decrease to about 40% decrease, about 15% decrease to about 35% decrease, about 15% decrease to about 30% decrease, about 15% decrease to about 25% decrease, about 15% decrease to about 20% decrease, about 20% decrease to about 99% decrease, an about 20% decrease to about 95% decrease, about 20% decrease to about 90% decrease, about 20% decrease to about 85% decrease, about 20% decrease to about 80% decrease, about 20% decrease to about 75% decrease, about 20% to about 70% decrease, about 20% decrease to about 65% decrease, about 20% decrease to about 60% decrease, about 20% decrease to about 55% decrease, about 20% decrease to about 50% decrease, about 20% decrease to about 45% decrease, about 20% decrease to about 40% decrease, about 20% decrease to about 35% decrease, about 20% decrease to about 30% decrease, about 20% decrease to about 25% decrease, about 25% decrease to about 99% decrease, an about 25% decrease to about 95% decrease, about 25% decrease to about 90% decrease, about 25% decrease to about 85% decrease, about 25% decrease to about 80% decrease, about 25% decrease to about 75% decrease, about 25% to about 70% decrease, about 25% decrease to about 65% decrease, about 25% decrease to about 60% decrease, about 25% decrease to about 55% decrease, about 25% decrease to about 50% decrease, about 25% decrease to about 45% decrease, about 25% decrease to about 40% decrease, about 25% decrease to about 35% decrease, about 25% decrease to about 30% decrease, about 30% decrease to about 99% decrease, an about 30% decrease to about 95% decrease, about 30% decrease to about 90% decrease, about 30% decrease to about 85% decrease, about 30% decrease to about 80% decrease, about 30% decrease to about 75% decrease, about 30% to about 70% decrease, about 30% decrease to about 65% decrease, about 30% decrease to about 60% decrease, about 30% decrease to about 55% decrease, about 30% decrease to about 50% decrease, about 30% decrease to about 45% decrease, about 30% decrease to about 40% decrease, about 30% decrease to about 35% decrease, about 35% decrease to about 99% decrease, an about 35% decrease to about 95% decrease, about 35% decrease to about 90% decrease, about 35% decrease to about 85% decrease, about 35% decrease to about 80% decrease, about 35% decrease to about 75% decrease, about 35% to about 70% decrease, about 35% decrease to about 65% decrease, about 35% decrease to about 60% decrease, about 35% decrease to about 55% decrease, about 35% decrease to about 50% decrease, about 35% decrease to about 45% decrease, about 35% decrease to about 40% decrease, about 40% decrease to about 99% decrease, an about 40% decrease to about 95% decrease, about 40% decrease to about 90% decrease, about 40% decrease to about 85% decrease, about 40% decrease to about 80% decrease, about 40% decrease to about 75% decrease, about 40% to about 70% decrease, about 40% decrease to about 65% decrease, about 40% decrease to about 60% decrease, about 40% decrease to about 55% decrease, about 40% decrease to about 50% decrease, about 40% decrease to about 45% decrease, about 45% decrease to about 99% decrease, an about 45% decrease to about 95% decrease, about 45% decrease to about 90% decrease, about 45% decrease to about 85% decrease, about 45% decrease to about 80% decrease, about 45% decrease to about 75% decrease, about 45% to about 70% decrease, about 45% decrease to about 65% decrease, about 45% decrease to about 60% decrease, about 45% decrease to about 55% decrease, about 45% decrease to about 50% decrease, about 50% decrease to about 99% decrease, an about 50% decrease to about 95% decrease, about 50% decrease to about 90% decrease, about 50% decrease to about 85% decrease, about 50% decrease to about 80% decrease, about 50% decrease to about 75% decrease, about 50% to about 70% decrease, about 50% decrease to about 65% decrease, about 50% decrease to about 60% decrease, about 50% decrease to about 55% decrease, about 55% decrease to about 99% decrease, an about 55% decrease to about 95% decrease, about 55% decrease to about 90% decrease, about 55% decrease to about 85% decrease, about 55% decrease to about 80% decrease, about 55% decrease to about 75% decrease, about 55% to about 70% decrease, about 55% decrease to about 65% decrease, about 55% decrease to about 60% decrease, about 60% decrease to about 99% decrease, an about 60% decrease to about 95% decrease, about 60% decrease to about 90% decrease, about 60% decrease to about 85% decrease, about 60% decrease to about 80% decrease, about 60% decrease to about 75% decrease, about 60% to about 70% decrease, about 60% decrease to about 65% decrease, about 65% decrease to about 99% decrease, an about 65% decrease to about 95% decrease, about 65% decrease to about 90% decrease, about 65% decrease to about 85% decrease, about 65% decrease to about 80% decrease, about 65% decrease to about 75% decrease, about 65% to about 70% decrease, about 70% decrease to about 99% decrease, an about 70% decrease to about 95% decrease, about 70% decrease to about 90% decrease, about 70% decrease to about 85% decrease, about 70% decrease to about 80% decrease, about 70% decrease to about 75% decrease, about 75% decrease to about 99% decrease, an about 75% decrease to about 95% decrease, about 75% decrease to about 90% decrease, about 75% decrease to about 85% decrease, about 75% decrease to about 80% decrease, about 80% decrease to about 99% decrease, an about 80% decrease to about 95% decrease, about 80% decrease to about 90% decrease, about 80% decrease to about 85% decrease, about 85% decrease to about 99% decrease, an about 85% decrease to about 95% decrease, about 85% decrease to about 90% decrease, about 90% decrease to about 99% decrease, an about 90% decrease to about 95% decrease, or about 95% decrease to about 99% decrease) in the number of senescent cells in the subject (e.g., a decrease in the number of senescent cells in one or more specific tissues involved and/or implicated in the aging-related disease or disorder in the subject), e.g., as compared to the number of senescent cells in the subject prior to treatment.

In some examples of these methods, the subject has been diagnosed or identified as having an infectious disease. Non-limiting examples of infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, and influenza virus. In some embodiments, these methods can result in a decrease in the infectious titer (e.g., viral titer) in a subject (e.g., as compared to the infectious titer in the subject prior to treatment). In some embodiments, these methods can result in a reduction in the number, severity, or frequency of one or more symptoms of the infectious disease (e.g., viral infection) in the subject (e.g., as compared to the number, severity, or frequency of the one or more symptoms of the infectious disease in the subject prior to treatment).

The term “subject” refers to any mammal. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine or primate animals) may be employed.

### Methods of Killing a Cancer Cell, an Infected Cell, or a Senescent Cell

Also provided herein are methods of killing a cancer cell (e.g., any of the exemplary types of cancer described herein or known in the art), an infected cell (e.g., a cell infected with any of the exemplary viruses described herein or known in the art), or a senescent cell (e.g., a senescent cancer cell, a senescent fibroblast, or a senescent endothelial cell) in a subject in need thereof (e.g., any of the exemplary subjects described herein or known in the art) that include administering to the subject a therapeutically effective amount of any of the multi-chain chimeric polypeptides described herein or any of the compositions (e.g., pharmaceutical compositions) described herein.

In some embodiments of these methods, the subject has been identified or diagnosed as having a cancer. Non-limiting examples of cancer include: solid tumor, hematological tumor, sarcoma, osteosarcoma, glioblastoma, neuroblastoma, melanoma, rhabdomyosarcoma, Ewing sarcoma, osteosarcoma, B-cell neoplasms, multiple myeloma, B-cell lymphoma, B-cell non-Hodgkin's lymphoma, Hodgkin's lymphoma, chronic lymphocytic leukemia (CLL), acute myeloid leukemia (AML), chronic myeloid leukemia (CML), acute lymphocytic leukemia (ALL), myelodysplastic syndromes (MDS), cutaneous T-cell lymphoma, retinoblastoma, stomach cancer, urothelial carcinoma, lung cancer, renal cell carcinoma, gastric and esophageal cancer, pancreatic cancer, prostate cancer, breast cancer, colorectal cancer, ovarian cancer, non-small cell lung carcinoma, squamous cell head and neck carcinoma, endometrial cancer, cervical cancer, liver cancer, and hepatocellular carcinoma.

In some examples of these methods, the subject has been identified or diagnosed as having an aging-related disease or condition. Non-limiting examples of aging-related diseases and conditions include Alzheimer's disease, aneurysm, cystic fibrosis, fibrosis in pancreatitis, glaucoma, hypertension, idiopathic pulmonary fibrosis, inflammatory bowel disease, intervertebral disc degeneration, macular degeneration, osteoarthritis, type 2 diabetes mellitus, adipose atrophy, lipodystrophy, atherosclerosis, cataracts, COPD, idiopathic pulmonary fibrosis, kidney transplant failure, liver fibrosis, loss of bone mass, myocardial infarction, sarcopenia, wound healing, alopecia, cardiomyocyte hypertrophy, osteoarthritis, Parkinson's disease, age-associated loss of lung tissue elasticity, macular degeneration, cachexia, glomerulosclerosis, liver cirrhosis, NAFLD, osteoporosis, amyotrophic lateral sclerosis, Huntington's disease, spinocerebellar ataxia, multiple sclerosis, and renal dysfunction.

In some examples of these methods, the subject has been diagnosed or identified as having an infectious disease. Non-limiting examples of an infectious disease include infection with human immunodeficiency virus, cytomegalovirus, adenovirus, coronavirus, rhinovirus, rotavirus, smallpox, herpes simplex virus, hepatitis B virus, hepatitis A virus, and hepatitis C virus, papillomavirus, and influenza virus.

### Senescent Cells

Senescence is a form of irreversible growth arrest accompanied by phenotypic changes, resistance to apoptosis and activation of damage-sensing signaling pathways. Cellular senescence was first described in cultured human fibroblast cells that lost their ability to proliferate, reaching permanent arrest after about 50 population doublings (referred to as the Hayflick limit). Senescence is considered a stress response that can be induced by a wide range of intrinsic and extrinsic insults, including oxidative and genotoxic stress, DNA damage, telomere attrition, oncogenic activation, mitochondrial dysfunction, or chemotherapeutic agents.

Senescent cells remain metabolically active and can influence the tissue hemostasis, disease and aging through their secretory phenotype. Senescence is considered as a physiologic process and is important in promoting wound healing, tissue homeostasis, regeneration, and fibrosis regulation. For instance, transient induction of senescent cells is observed during would healing and contributes to wound resolution. Perhaps one of the most important roles of senescence is its role in tumor suppression. However, the accumulation of senescent cells also drives aging- and aging-related diseases and conditions. The senescent phenotype also can trigger chronic inflammatory responses and consequently augment chronic inflammatory conditions to promote tumor growth. The connection between senescence and aging was initially based on observations that senescent cells accumulate in aged tissue. The use of transgenic models has enabled the detection of senescent cells systematically in many age-related pathologies. Strategies to selectively eliminate senescent cells has demonstrated that senescent cells can indeed play a causal role in aging and related pathologies.

Senescent cells display important and unique properties which include changes in morphology, chromatin organization, gene expression, and metabolism. There are several biochemical and functional properties associated with cellular senescence, such as (i) increased expression of p16 and p21, inhibitors of cyclin-dependent kinases, (ii) presence of senescence-associated β-galactosidase, a marker of lysosomal activity, (iii) appearance of senescence-associated heterochromatin foci and downregulation of lamin B1 levels, (iv) resistance to apoptosis caused by an increased expression of anti-apoptotic BCL-family protein, and (v) upregulation of CD26 (DPP4), CD36 (Scavenger receptor), forkhead box 4 (FOXO4), PD-L1, and secretory carrier membrane protein 4 (SCAMP4). Senescent cells also express an inflammatory signature, the so-called senescence-associated secretory phenotype (SASP). Through SASP, the senescent cells produce a wide range of inflammatory cytokines (IL-6, IL-8), growth factors (TGF-β), chemokines (CCL-2), and matrix metalloproteinases (MMP-3, MMP-9) that operate in a cell-autonomous manner to reinforce senescence (autocrine effects) and communicate with and modify the microenvironment (paracrine effects). SASP factors can contribute to tumor suppression by triggering senescence surveillance, an immune-mediated clearance of senescent cells. However, chronic inflammation is also a known driver of tumorigenesis, and accumulating evidence indicates that chronic SASP can also boost cancer and aging-related diseases.

The secretion profile of senescent cells is context dependent. For instance, the mitochondrial dysfunction-associated senescence (MiDAS), induced by different mitochondrial dysfunction in human fibroblasts, led to the appearance of a SASP that was deficient in IL-1− dependent inflammatory factors. A decrease in the NAD+/NADH ratio activated AMPK signaling which induced MiDAS through the activation of p53. As a result, p53 inhibited NF-κB signaling which is a crucial inducer of pro-inflammatory SASP. In contrast, the cellular senescence caused by persistent DNA damage in human cells induced an inflammatory SASP, which was dependent on the activation of ataxia-telangiectasia mutated (ATM) kinase but not on that of p53. In particular, the expression and secretion levels of IL-6 and IL-8 were increased. It was also demonstrated that cellular senescence caused by the ectopic expression p16INK4a and p21CIP1 induced the senescent phenotype in human fibroblasts without an inflammatory SASP indicating that the growth arrest itself did not stimulate SASP.

One of the most defining characteristics of senescence is stable growth arrest. This is achieved by two important pathways, the p16/Rb and the p53/p21, both of which are central in tumor suppression. DNA damage results in: (1) high deposition of 7H2Ax (histone coding gene) and 53BP1 (involved in DNA damage response) in chromatin: this leads to activation of a kinase cascade eventually resulting in p53 activation, and (2) activation of p16INK4a and ARF (both encoded by CDKN2A) and P15INK4b (encoded by CDKN2B): p53 induces transcription of cyclin-dependent kinase inhibitor (p21) and along with both p161NK4a and p15INK4b block genes for cell cycle progression (CDK4 and CDK6). This eventually leads to hypophosphorylation of Retinoblastoma protein (Rb) and cell cycle arrest at the G1 phase.

Selectively killing senescent cells has been shown to significantly improve the health span of mice in the context of normal aging and ameliorates the consequences of age-related disease or cancer therapy (Ovadya, J Clin Invest. 128(4):1247-1254, 2018). In nature, the senescent cells are normally removed by the innate immune cells. Induction of senescence not only prevents the potential proliferation and transformation of damaged/altered cells, but also favors tissue repair through the production of SASP factors that function as chemoattractants mainly for Natural Killer (NK) cells (such as IL-15 and CCL2) and macrophages (such as CFS-1 and CCL2). These innate immune cells mediate the immunosurveillance mechanism for eliminating stressed cells. Senescent cells usually up-regulate the NK-cell activating receptor NKG2D and DNAM-1 ligands, which belong to a family of stress-inducible ligands: an important component of the frontline immune defense against infectious diseases and malignancies. Upon receptor activation, NK cells can then specifically induce the death of senescent cells through their cytolytic machinery. A role for NK cells in the immune surveillance of senescent cells has been pointed out in liver fibrosis (Sagiv, Oncogene 32(15): 1971-1977, 2013), hepatocellular carcinoma (Iannello, J Exp Med 210(10): 2057-2069, 2013), multiple myeloma (Soriani, Blood 113(15): 3503-3511, 2009), and glioma cells stressed by dysfunction of the mevalonate pathway (Ciaglia, Int J Cancer 142(1): 176-190, 2018). Endometrial cells undergo acute cellular senescence and do not differentiate into decidual cells. The differentiated decidual cells secrete IL-15 and thereby recruit uterine NK cells to target and eliminate the undifferentiated senescent cells thus helping to re-model and rejuvenate the endometrium (Brighton, Elife 6: e31274, 2017). With a similar mechanism, during liver fibrosis, p53-expressing senescent liver satellite cells skewed the polarization of resident Kupfer macrophages and freshly infiltrated macrophages toward the pro-inflammatory M1 phenotype, which display senolytic activity. F4/80+ macrophages have been shown to play a key role in the clearance of mouse uterine senescent cells to maintain postpartum uterine function.

Senescent cells recruit NK cells by mainly upregulating ligands to NKG2D (expressed on NK cells), chemokines, and other SASP factors. In vivo models of liver fibrosis have shown effective clearance of senescent cells by activated NK cells (Krizhanovsky, Cell 134(4): 657-667, 2008). Studies have described various models to study senescence including liver fibrosis (Krizhanovsky, Cell 134(4): 657-667, 2008), osteoarthritis (Xu, J Gerontol A Biol Sci Med Sci 72(6): 780-785, 2017), and Parkinson's disease (Chinta, Cell Rep 22(4): 930-940, 2018). Animal models for studying senescent cells are described in: Krizhanovsky, Cell 134(4): 657-667, 2008; Baker, Nature 479(7372): 232-236, 2011; Farr, Nat Med 23(9): 1072-1079, 2017; Bourgeois, FEBS Lett 592(12): 2083-2097, 2018; Xu, Nat Med 24(8): 1246-1256, 2018).

### Additional Therapeutic Agents

Some embodiments of any of the methods described herein can further include administering to a subject (e.g., any of the subjects described herein) a therapeutically effective amount of one or more additional therapeutic agents. The one or more additional therapeutic agents can be administered to the subject at substantially the same time as the multi-chain chimeric polypeptide (e.g., any of the multi-chain chimeric polypeptides described herein) or immune cell (e.g., administered as a single formulation or two or more formulations to the subject). In some embodiments, one or more additional therapeutic agents can be administered to the subject prior to administration of the multi-chain chimeric polypeptide (e.g., any of the multi-chain chimeric polypeptides described herein) or immune cell. In some embodiments, one or more additional therapeutic agents can be administered to the subject after administration of the multi-chain chimeric polypeptide (e.g., any of the multi-chain chimeric polypeptides described herein) or immune cell to the subject.

Non-limiting examples of additional therapeutic agents include: anti-cancer drugs, activating receptor agonists, immune checkpoint inhibitors, agents for blocking HLA-specific inhibitory receptors, Glucogen Synthase Kinase (GSK) 3 inhibitors, and antibodies.

Non-limiting examples of anticancer drugs include antimetabolic drugs (e.g., 5-fluorouracil (5-FU), 6-mercaptopurine (6-MP), capecitabine, cytarabine, floxuridine, fludarabine, gemcitabine, hydroxycarbamide, methotrexate, 6-thioguanine, cladribine, nelarabine, pentostatin, or pemetrexed), plant alkaloids (e.g., vinblastine, vincristine, vindesine, camptothecin, 9-methoxycamptothecin, coronaridine, taxol, naucleaorals, diprenylated indole alkaloid, montamine, schischkiniin, protoberberine, berberine, sanguinarine, chelerythrine, chelidonine, liriodenine, clivorine, β-carboline, antofine, tylophorine, cryptolepine, neocryptolepine, corynoline, sampangine, carbazole, crinamine, montanine, ellipticine, paclitaxel, docetaxel, etoposide, tenisopide, irinotecan, topotecan, or acridone alkaloids), proteasome inhibitors (e.g., lactacystin, disulfiram, epigallocatechin-3-gallate, marizomib (salinosporamide A), oprozomib (ONX-0912), delanzomib (CEP-18770), epoxomicin, MG132, beta-hydroxy beta-methylbutyrate, bortezomib, carfilzomib, or ixazomib), antitumor antibiotics (e.g., doxorubicin, daunorubicin, epirubicin, mitoxantrone, idarubicin, actinomycin, plicamycin, mitomycin, or bleomycin), histone deacetylase inhibitors (e.g., vorinostat, panobinostat, belinostat, givinostat, abexinostat, depsipeptide, entinostat, phenyl butyrate, valproic acid, trichostatin A, dacinostat, mocetinostat, pracinostat, nicotinamide, cambinol, tenovin 1, tenovin 6, sirtinol, ricolinostat, tefinostat, kevetrin, quisinostat, resminostat, tacedinaline, chidamide, or selisistat), tyrosine kinase inhibitors (e.g., axitinib, dasatinib, encorafinib, erlotinib, imatinib, nilotinib, pazopanib, and sunitinib), and chemotherapeutic agents (e.g., all-trans retinoic acid, azacitidine, azathioprine, doxifluridine, epothilone, hydroxyurea, imatinib, teniposide, tioguanine, valrubicin, vemurafenib, and lenalidomide). Additional examples of chemotherapeutic agents include alkylating agents, e.g., mechlorethamine, cyclophosphamide, chlorambucil, melphalan, ifosfamide, thiotepa, hexamethylmelamine, busulfan, altretamine, procarbazine, dacarbazine, temozolomide, carmustine, lumustine, streptozocin, carboplatin, cisplatin, and oxaliplatin.

Non-limiting examples of activating receptor agonists include any agonists for activating receptors which activate and enhance the cytotoxicity of NK cells, including anti-CD16 antibodies (e.g., anti-CD16/CD30 bispecific monoclonal antibody (BiMAb)) and Fc-based fusion proteins. Non-limiting examples of checkpoint inhibitors include anti-PD-1 antibodies (e.g., MEDIO680), anti-PD-L1 antibodies (e.g., BCD-135, BGB-A333, CBT-502, CK-301, CS1001, FAZ053, KN035, MDX-1105, MSB2311, SHR-1316, anti-PD-L1/CTLA-4 bispecific antibody KN046, anti-PD-L1/TGFβRII fusion protein M7824, anti-PD-L1/TIM-3 bispecific antibody LY3415244, atezolizumab, or avelumab), anti-TIM3 antibodies (e.g., TSR-022, Sym023, or MBG453) and anti-CTLA-4 antibodies (e.g., AGEN1884, MK-1308, or an anti-CTLA-4/OX40 bispecific antibody ATOR-1015). Non-limiting examples of agents for blocking HLA-specific inhibitory receptors include monalizumab (e.g., an anti-HLA-E NKG2A inhibitory receptor monoclonal antibody). Non-limiting examples of GSK3 inhibitor include tideglusib or CHIR99021. Non-limiting examples of antibodies that can be used as additional therapeutic agents include anti-CD26 antibodies (e.g., YS110), anti-CD36 antibodies, and any other antibody or antibody construct that can bind to and activate an Fc receptor (e.g., CD16) on a NK cell. In some embodiments, an additional therapeutic agent can be insulin or metformin.

### EXAMPLES

The invention is further described in the following examples, which do not limit the scope of the invention described in the claims.

### Example 1: Increased IFN-γ and Minimal Expression of IL-6

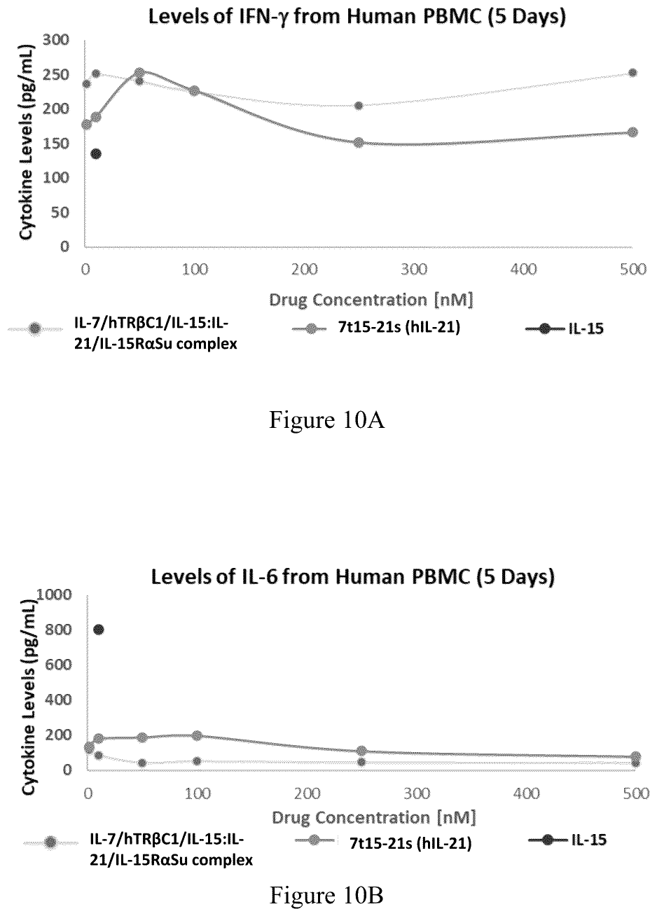

Frozen human leukocytes which were previously obtained from healthy donors and frozen PBMC were thawed and counted. Cells were resuspended in 2×10⁶/mL in 24 wells flat bottom plate in 0.2 mL of complete media (RPMI 1640 (Gibco) supplemented with 2 mM L-glutamine (Thermo Life Technologies), penicillin (Thermo Life Technologies), streptomycin (Thermo Life Technologies), and 10% FBS (Hyclone)). Cells were stimulated with the following molecules: low dose IL-15 (LD IL-15 10 nM), IL-7/hTRβC1/IL-15:hIL-21/IL-15RαSu complex (1-500 nM) or 7t15-21s (1-500 nM) for 5 days. 7t15-21s is described in International Publication No. WO 2020/047299. After day 5, the cells supernatant was collected, harvested and frozen in a −80-degree freezer. Later the supernatant was thawed and analyzed by Luminex ELISA standard protocol as per the protocol for human procarta plex IFN-γ and IL-6. See FIGS. 10A and 10B.

IL-7/hTRβC1/IL-15:hIL-21/IL-15RαSu complex induced higher IFN-γ production than 7t15-21s in human PBMCs, with minimal IL-6 expression. This suggests a favorable safety profile and reduced risk of cytokine release syndrome in the context of CAR-T cell therapy.

### Example 2: IL-7/hTRβC1/IL-15:hIL-21/IL-15RαSu Complex Treatment Slows B16F10 Tumor Growth in C57BL/6 Mice

To evaluate the antitumor efficacy of IL-7/hTRβC1/IL-15:hIL-21/IL-15RαSu complex, 5×10⁵ mouse B16F10 melanoma cells (CRL-6475, ATCC) were subcutaneously injected into C57BL/6 mice (day 0) followed by subcutaneous injection of PBS, 7t15-21s or IL-7/hTRβC1/IL-15:hIL-21/IL-15RαSu complex (9 mg/kg) on day 8 after tumor implantation. Tumor growth was monitored by caliper measurement, and tumor volume was calculated using the formula V=(L×W2)/2, where L is the largest tumor diameter and W is the perpendicular tumor diameter.

Tumor volume was measured every other day and mice with tumors ≥4000 mm³ were sacrificed per IACUC regulations.

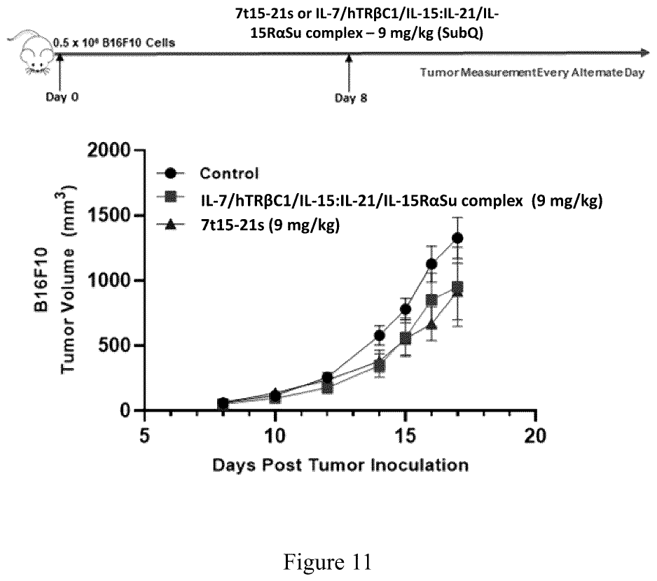

Both 7t15-21s and IL-7/hTRβC1/IL-15:hIL-21/IL-15RαSu complex treatments were able to reduce growth of B16F10 tumors in C57BL/6 mice. See FIG. 11.

### Example 3: Biochemical Characterization of IL-7/hTRβC1/IL-15:hIL-21/IL-15RαSu Complex Compared with 7t15-21s

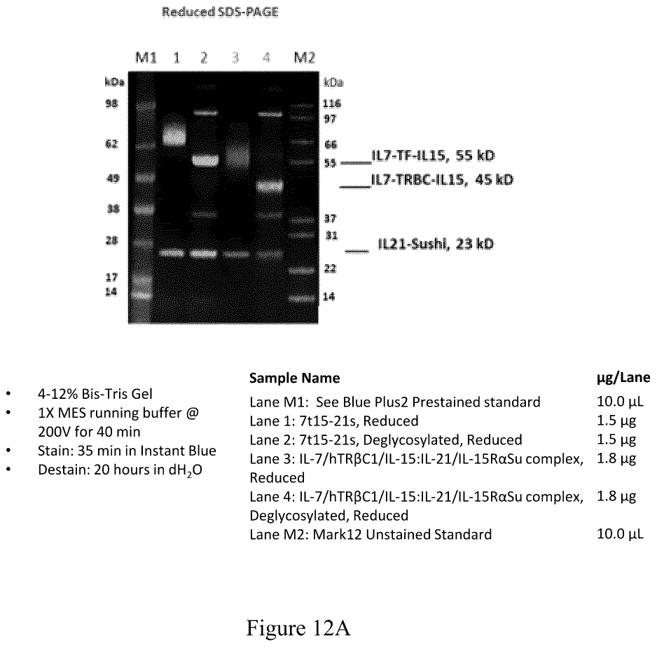

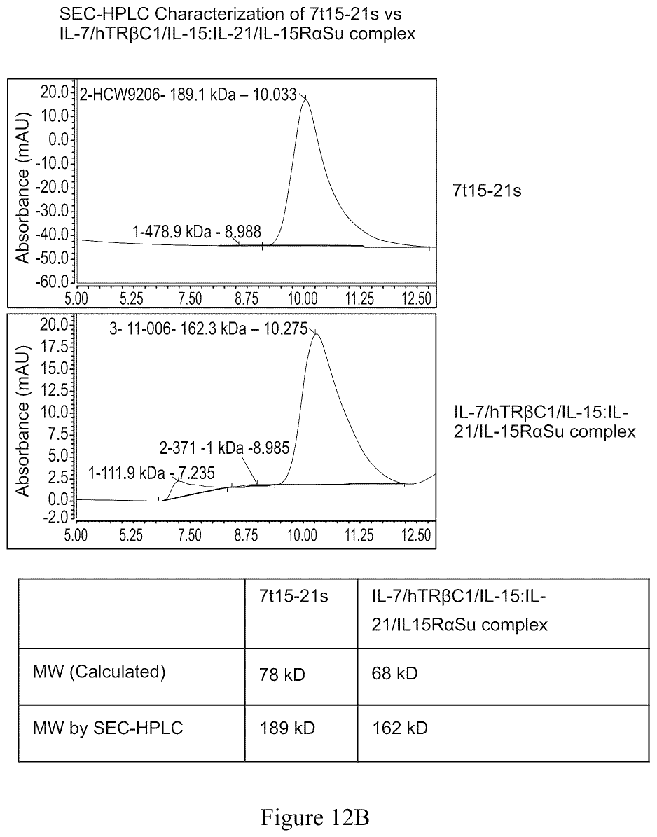

SDS-PAGE was used to examine the molecular weight of protein components under denatured condition (FIG. 12A). Protein standards were included in the gel as molecular weight references. Sample deglycosylation was performed to remove glycans from the sample prior to resolving on the gel to accurately determine the molecular weight of the sample. Purified IL-7/hTRβC1/IL-15:hIL-21/IL-15RαSu complex was resolved into two bands under deglycosylated and reduced conditions: 45 kDa (IL-7-hTRβC1-IL-15) and 23 kDa (IL-21-IL-15RαSushi). SEC-HPLC is used to determine the purity and size of samples under native condition (FIG. 12B). The elution time is correlated to the molecular size: the smaller size, the longer elution time.

SDS-PAGE analysis of purified IL-7/hTRβC1/IL-15:IL-21/IL-15RαSu complex revealed two polypeptides at 45 kDa and 23 kDa after deglycosylation, consistent with the expected molecular weights of IL-7-hTRβC1-IL-15 (45 kDa) and hIL-21-IL-15RαSushi (23 kDa), while size exclusion chromatography indicated a native molecular weight of 162 kDa, suggesting a glycosylated dimeric structure or an irregularly shaped protein.

### Example 4: Biological Activities of Each Cytokine in IL-7/hTRβC1/IL-15:hIL-21/IL-15RαSu Complex Using HEK Reporter and B9 Cell Lines

HEK-Blue IL-2 and HEK-Blue IL-7 reporter cells (5×10⁴ cells/well) were seeded in 200 μL IMDM:10% heat-inactivated FBS media. Cells were stimulated with either 7t15-21s or IL-7/hTRβC1/IL-15:hIL-21/IL-15RαSu complex and incubated overnight at 37° C./5% CO₂. In the reporter cell supernatant, QUANTI-Blue (InvivoGen) solution was added, and incubated for 1-3 hours at 37° C. Cytokine activity was assessed by measuring absorbance at 620 nm.

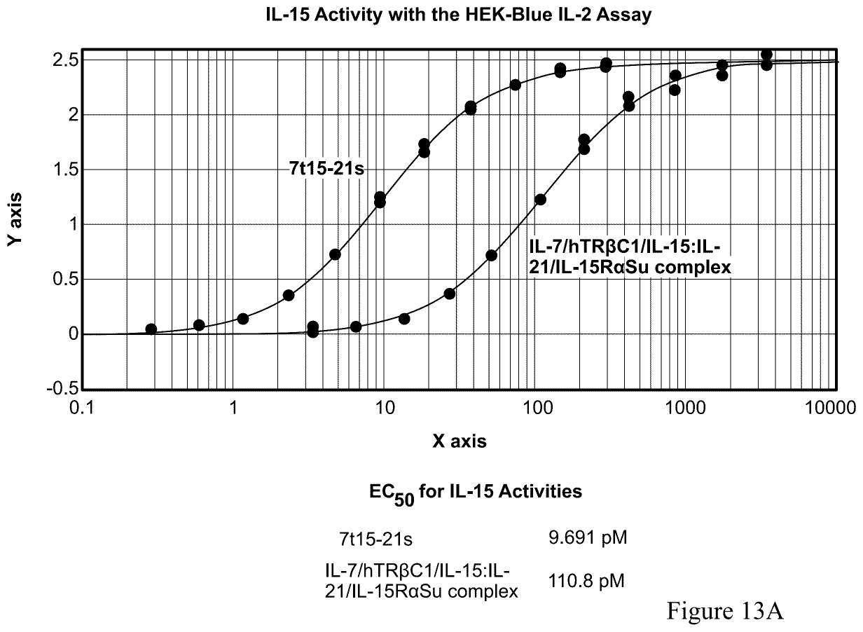

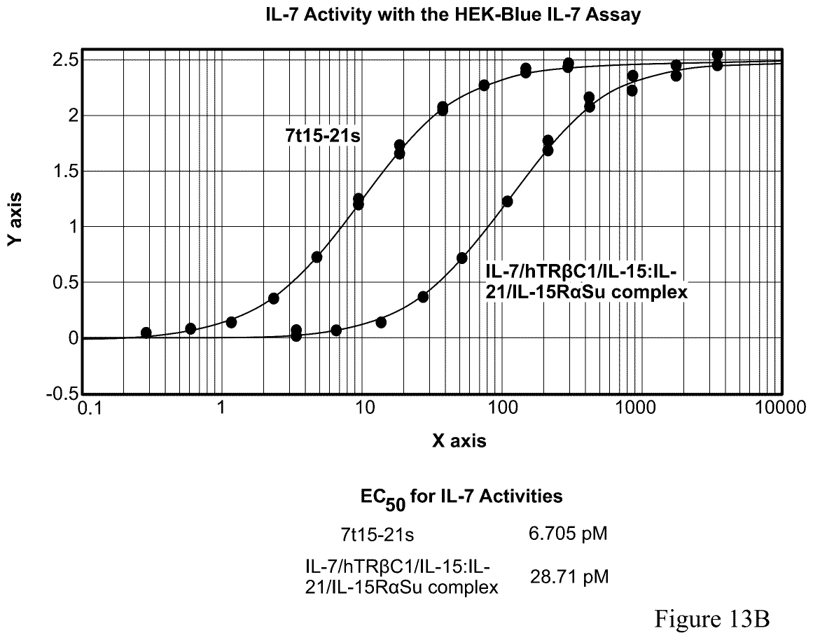

The biological activity of IL-7/hTRβC1/IL-15:IL-21/IL-15RαSu complex on HEK cells was assessed using dose-response curves, revealing varying EC50 values and cytokine activity differences when compared with 7t15-21s treatment across IL-7 and IL-15 cytokine-dependent reporter cell lines (see FIGS. 13A and 13B).

Additionally, B9 cells (0.4×10⁵ cells/well) were seeded in 200 μL IMIDM:10% heat inactivated FBS media. Cells were stimulated with either 7t15-21s or IL-7/hTRβC1/IL-15:IL-21/IL-15RαSu complex and incubated for 5 days at 37° C./5% CO₂. Cytokine activity was assessed by measuring absorbance at 570 nm using presto blue.

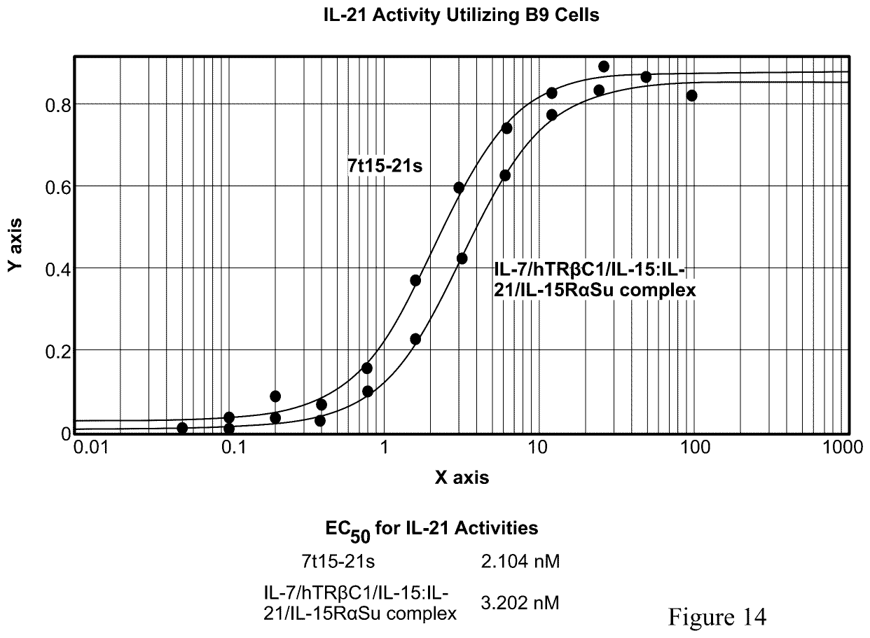

The biological activity of IL-7/hTRβC1/IL-15:IL-21/IL-15RαSu complex on B9 cells was assessed using dose-response curves, revealing varying EC50 values and cytokine activity differences when compared with 7t15-21s treatment using an IL-21 cytokine-dependent cell line (see FIG. 14).

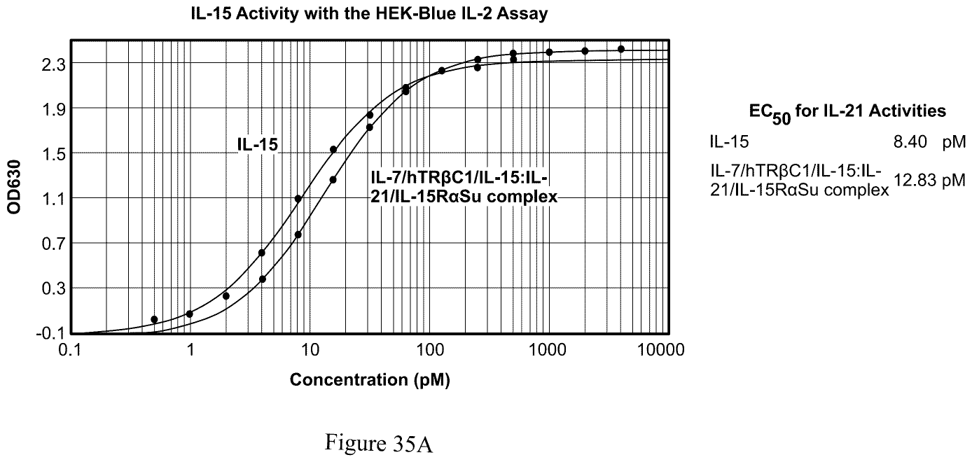

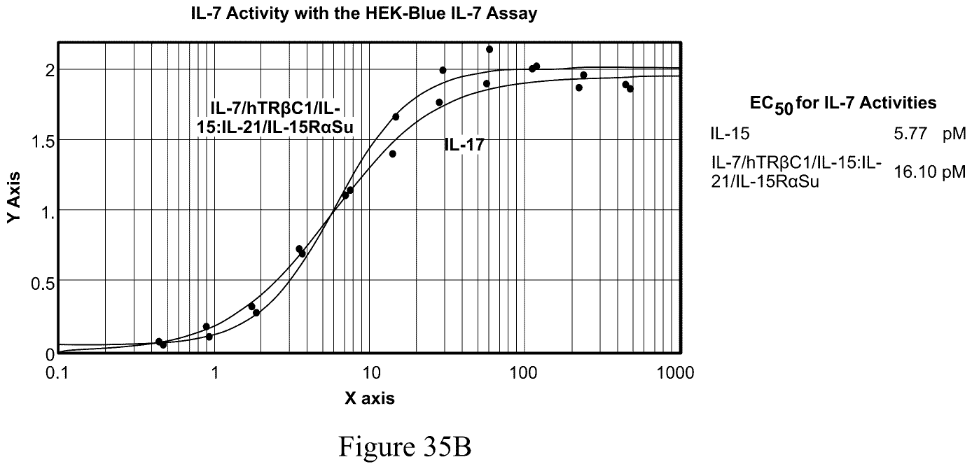

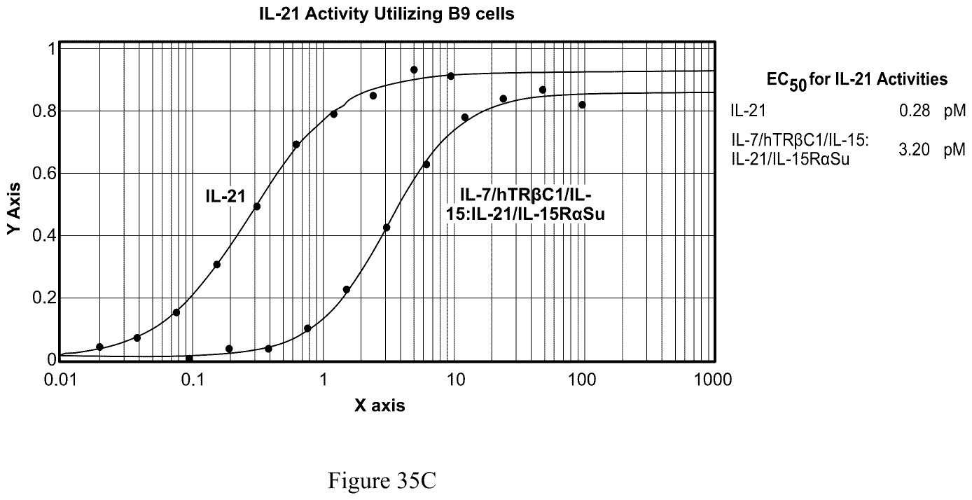

The biological activity of IL-7/hTRβC1/IL-15:hIL-21/IL-15RαSu complex on HEK and B9 cell lines was also assessed using dose-response curves to determine the concentration needed to activate specific cytokine-dependent HEK-Blue reporter and B9 cell lines. The EC50 values revealed similar activities for IL-7 and IL-15, while IL-21 exhibited ten-fold less activity. See FIGS. 35A-C.

### Example 5: IL-7/hTRβC1/IL-15:hIL-21/IL-15RαSu Complex Treatment Induces CD8⁺ T Cell and NK Cell Responses

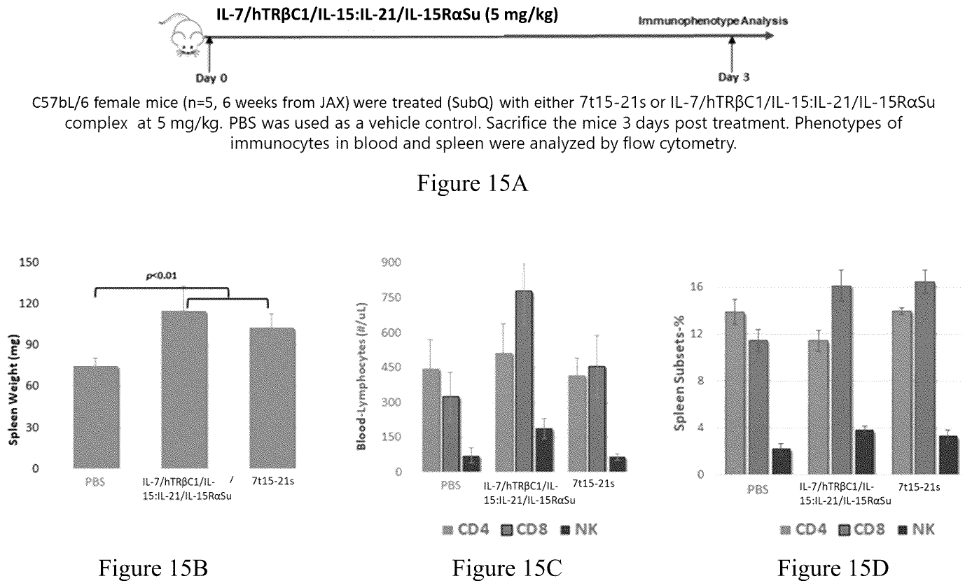

Female 6-8-week-old C57BL/6 mice were subcutaneously injected with 7t15-21s, IL-7/hTRβC1/IL-15:hIL-21/IL-15RαSu complex or PBS as a control (see FIG. 15A). After day 3 post-treatment, mice were humanely euthanized. Blood was obtained from tail vein. Spleens were collected, weighed (FIG. 15B), and used to prepare splenocytes (FIG. 15D). Splenocytes and blood cells were stained with fluorochrome-conjugated anti-CD4, -CD8, and -NK1.1 antibodies and the percentages of CD4⁺ T cells, CD8⁺ T cells and NK1.1+(NK) cells were analyzed by flow cytometry. FIGS. 15A-15C show IL-7/hTRβC1/IL-15:hIL-21/IL-15RαSu complex treatment induces CD8⁺ T cell and NK cell responses in spleens and blood of C57BL/6 mice.

Spleen weight was significantly higher in mice treated with IL-7/hTRβC1/IL-15:hIL-21/IL-15RαSu complex compared to control mice. IL-7/hTRβC1/IL-15:hIL-21/IL-15RαSu complex induced proliferation of lymphocytes (CD4, CD8, NK) in blood and spleen. The results demonstrate that IL-7/hTRβC1/IL-15:hIL-21/IL-15RαSu complex treatment was capable of inducing increased spleen weights and increased percentages of CD8⁺ T cells and NK1.1+(NK) cells in the blood and spleens of C57BL/6 mice. IL-7/hTRβC1/IL-15:IL-21/IL-15RαSu complex treatment showed better immunostimulatory effects on blood CD8⁺ T cells and NK1.1⁺ (NK) cells than was observed in 7t15-21s -treated mice. See FIGS. 15A-15D.

### Example 6: IL-7/hTRβC1/IL-15:hIL-21/IL-15RαSu Complex Treatment Induces Granzyme-B, Ki67, and CD44 in CD8⁺ T cells and NK cells in blood of C57BL/6 mice

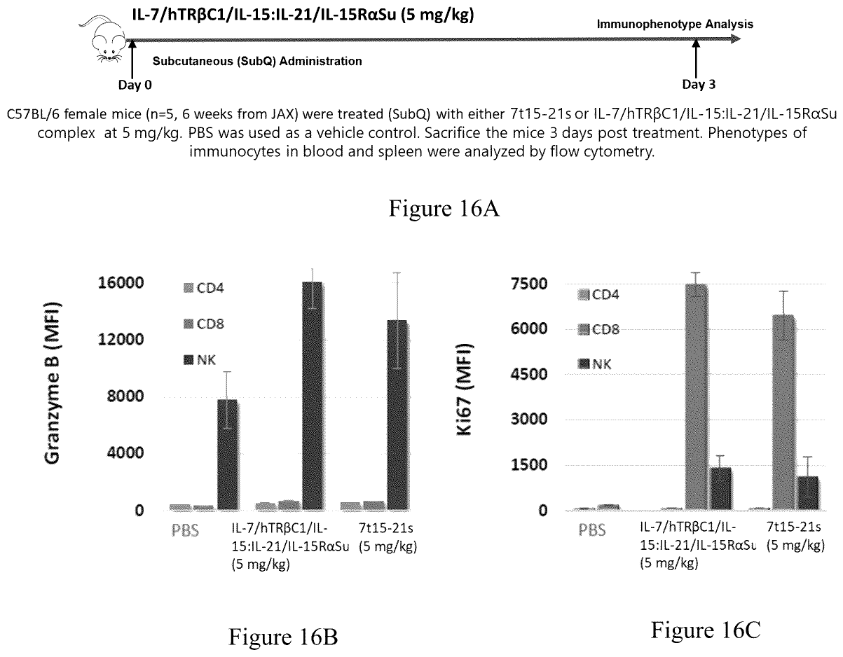

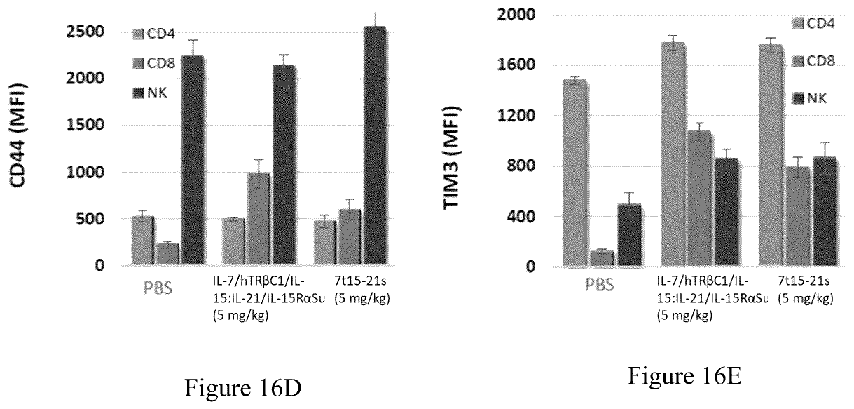

Female 6-8-week-old C57BL/6 mice were subcutaneously injected with 7t15-21s and IL-7/hTRβC1/IL-15:hIL-21/IL-15RαSu complex and PBS as a control (see FIG. 16A). Mice were humanely euthanized 3 days post-treatment. Blood cells were stained with fluorochrome-conjugated anti-CD4, -CD8, -NK1.1, CD44 (memory) and TIM3 (check point marker) antibodies and intracellularly stained for granzyme B (cytotoxicity) and Ki67 (proliferation marker) using fluorochrome-conjugated Abs. The mean fluorescent intensity (MFI) of CD44, granzyme B, Ki67, and TIM3was assessed by flow cytometry (see FIGS. 16B-16D).

The results demonstrate that IL-7/hTRβC1/IL-15:hIL-21/IL-15RαSu complex treatment was capable of increasing granzyme B expression in blood NK cells, CD44 expression in CD8+ T cells, Ki67 and TIM3 expression in both CD8⁺ T cells and NK cells in the blood of C57BL/6 mice. IL-7/hTRβC1/IL-15:hIL-21/IL-15RαSu complex treatment showed better induction of NK cell granzyme B expression and CD8⁺ T cell CD44 expression in blood than was observed in 7t15-21s -treated mice. See FIGS. 16A-16D.

### Example 7: IL-7/hTRβC1/IL-15:hIL-21/IL-15RαSu Complex Treatment Induces Granzyme-B, Ki67, and CD44 in CD8⁺ T Cells and NK Cells in Splenocytes of C57BL/6 Mice

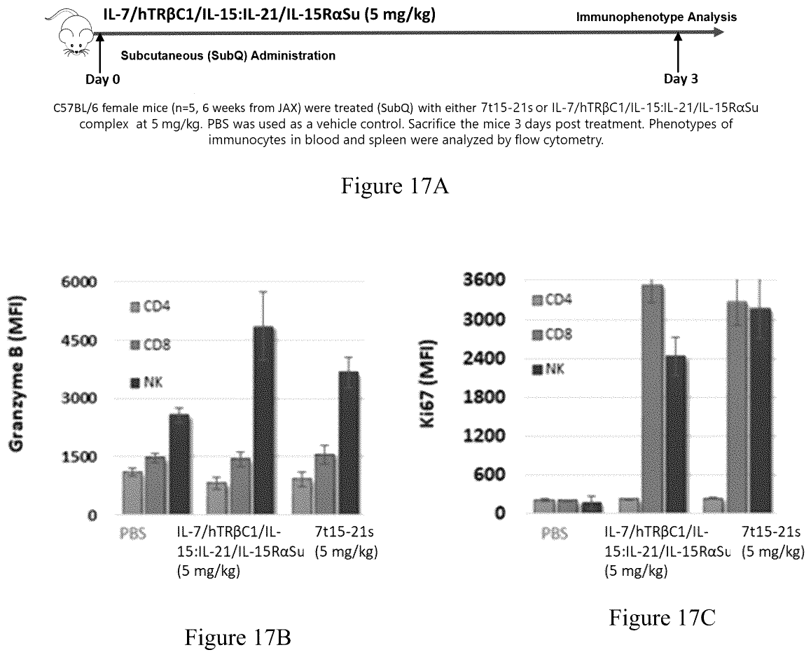

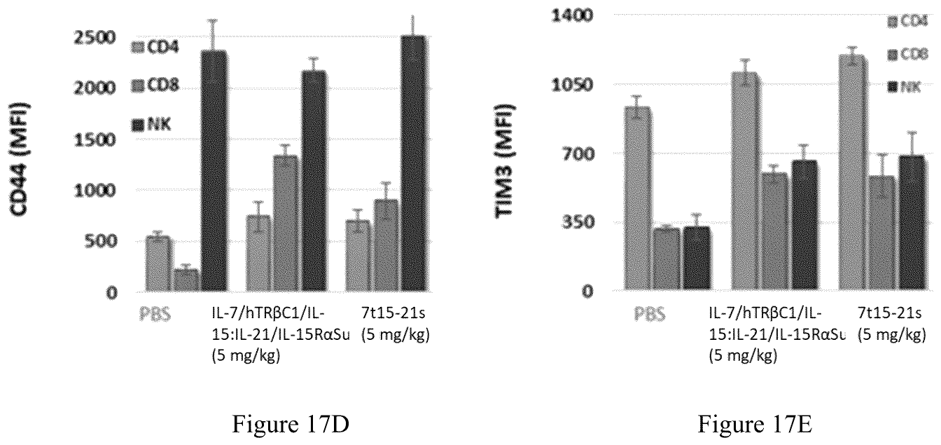

Female 6-8-week-old C57BL/6 mice were subcutaneously injected with 7t15-21s, IL-7/hTRβC1/IL-15:hIL-21/IL-15RαSu complex, or PBS as a control. Mice were humanely euthanized 3 days post-treatment. Spleens were collected and splenocytes were prepared. Splenocytes were stained with fluorochrome-conjugated anti-CD4, -CD8, -NK1.1, CD44 (memory) and TIM3 (check point marker) antibodies and intracellularly stained for CD44 (memory), granzyme B (cytotoxicity), Ki67 (proliferation marker) and TIM3 (check point marker) using fluorochrome-conjugated Abs. The mean fluorescent intensity (MFI) of CD44, granzyme B, Ki67, and TIM3 was assessed by flow cytometry (see FIGS. 17A-17D).

The results demonstrate that IL-7/hTRβC1/IL-15:hIL-21/IL-15RαSu complex treatment was capable of increasing granzyme B expression in blood NK cells, proliferation (Ki67) in both CD8⁺ T cells and NK cells, CD44 expression in T cells, and TIM3 expression in both T cells and NK cells in splenocytes of C57BL/6 mice. IL-7/hTRβC1/IL-15:hIL-21/IL-15RαSu complex treatment showed better induction of NK cell granzyme B expression and CD8⁺ T cell CD44 expression in splenocytes than was observed in 7t15-21s -treated mice.

### Example 8: Increased Lymphocytes (CD3⁺) Proliferation in Human PBMCs Stimulated with IL-7/hTRβC1/IL-15:hIL-21/IL-15RαSu Complex In Vitro

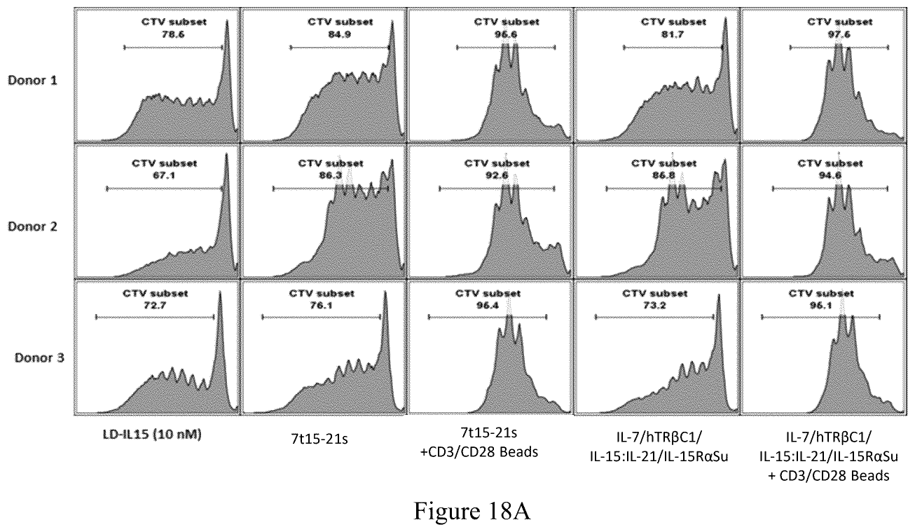

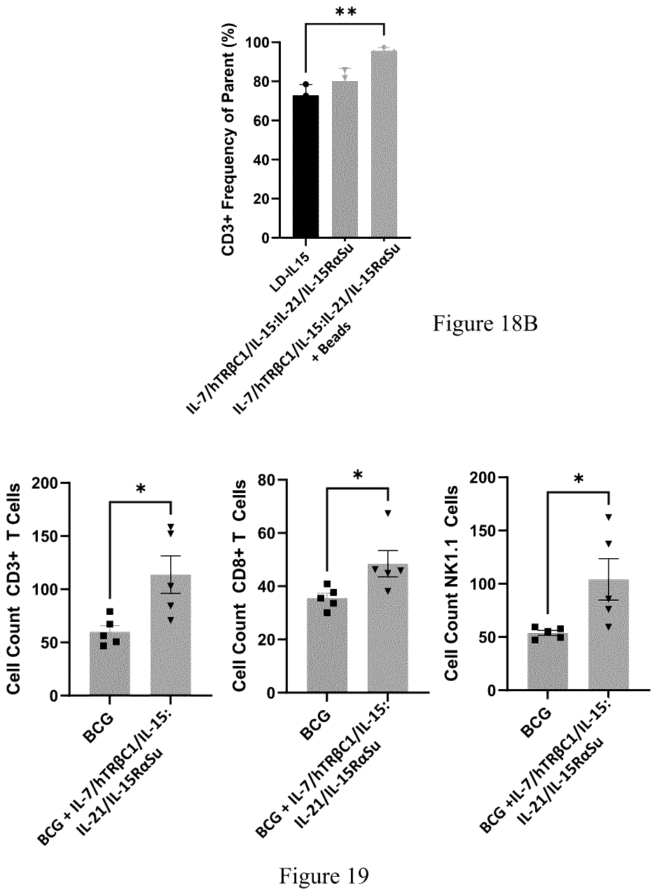

Frozen human leukocytes which were previously obtained from healthy donors and frozen PBMC were thawed and counted. Cells were labelled with cell trace violet (CTV) as per manufacturer's protocol. Cells were resuspended in 2×10⁶/mL in 24 wells flat bottom plate in 0.2 mL of complete media (RPMI 1640 (Gibco) supplemented with 2 mM L-glutamine (Thermo Life Technologies), penicillin (Thermo Life Technologies), streptomycin (Thermo Life Technologies), and 10% FBS (Hyclone)). Cells were stimulated for 7 days with the following molecules: low dose IL-15 (LD IL-15 10 nM), IL-7/hTRβC1/IL-15:IL-21/IL-15RαSu complex (100 nM) or 7t15-21s (100 nM) with or without CD3/CD28 beads. Cells were replenished with fresh media and test molecules every alternate day until day 7. At day 7, cells were harvested and stained for surface marker CD3. After surface staining, cells were washed (1500 RPM for 5 minutes at room temperature) in FACS buffer (1× PBS (Hyclone) with 0.5% BSA (EMD Millipore) and 0.001% Sodium Azide (Sigma)). After two washes, cells were analyzed by Flow Cytometry (Celesta-BD Bioscience) for cell proliferation gated on CD3⁺ cells (FIGS. 18A-B).

Results show that there was little or no difference in the proliferation of CTV⁺ stained CD3⁺ T cells after stimulation with either 7t15-21s or IL-7/hTRβC1/IL-15:hIL-21/IL-15RαSu complex. Both molecules promoted expansion of CD3⁺ T cells by day 7. Addition of CD3/CD28 beads further increased CD3⁺ T cell proliferation. IL-7/hTRβC1/IL-15:hIL-21/IL-15RαSu complex effectively expanded CD3⁺ T cells in the presence of CD3/CD28 stimulation compared to IL-15.

### Example 9: IL-7/hTRβC1/IL-15:hIL-21/IL-15RαSu Complex Treatment in Combination with Bacillus Calmette-Guerin (BCG)

To evaluate the effects of intravesical IL-7/hTRβC1/IL-15:hIL-21/IL-15RαSu complex in combination with standard of care BCG treatment on immune infiltration in the bladder, C57BL/6 mice were anesthetized with ip injection of ketamine, and a polyurethane 24G×3/4″ catheter was inserted transurethrally into the bladder. BCG with and without IL-7/hTRβC1/IL-15:hIL-21/IL-15RαSu complex was then instilled into the bladder (0.2 g in 50 μL per dose; BCG TICE strain at 3×10⁶ colony-forming units in 50 μL per dose) and maintained for 60 min. Heating pads were used to maintain core body temperature throughout implantation and recovery.

Next day bladder tissue was harvested and processed into single-cell suspensions. Cells were stained for surface markers CD3, CD16, CD56, and CD8. After surface staining, cells were washed (1500 RPM for 5 minutes at room temperature) in FACS buffer (1× PBS (Hyclone) with 0.5% BSA (EMD Millipore) and 0.001% Sodium Azide (Sigma)). After two washes, cells were analyzed by Flow Cytometry (Celesta-BD Bioscience) (FIG. 19).

Results show that following intravesical treatment with IL-7/hTRβC1/IL-15:IL-21/IL-15RαSu complex and standard BCG, increased NK and CD8⁺ T cell counts were observed in the bladder of C57BL/6 mice.

### Example 10: Increased Expression of IFN-γ and Granzyme-β in Lymphocytes

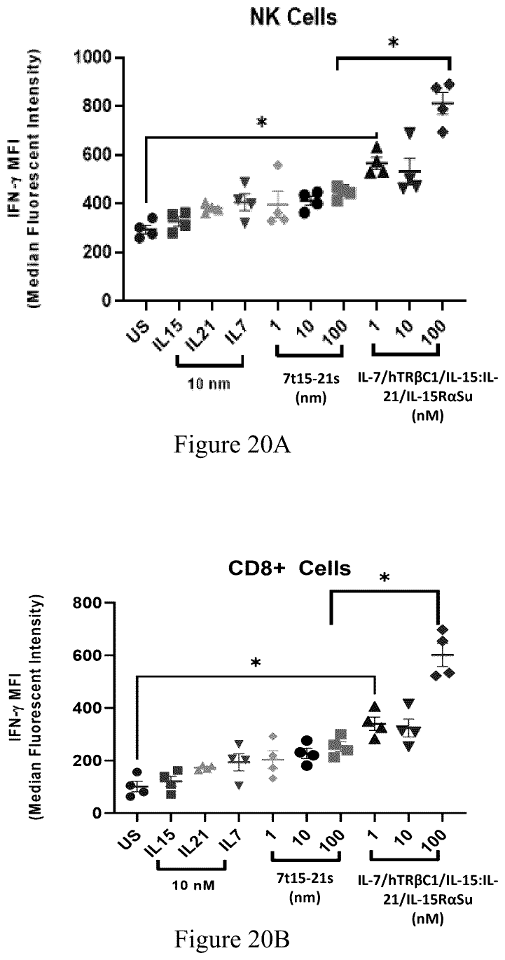

Fresh human leukocytes were obtained from healthy donors and PBMC were isolated. Cells were counted and resuspended in 2×10⁶/mL in 24 wells flat bottom plate in 2 mL of complete media (RPMI 1640 (Gibco) supplemented with 2 mM L-glutamine (Thermo Life Technologies), penicillin (Thermo Life Technologies), streptomycin (Thermo Life Technologies), and 10% FBS (Hyclone)). Cells were stimulated overnight with the following molecules: media alone, IL-7, IL-15 or IL-21, IL-7/hTRβC1/IL-15:IL-21/IL-15RαSu complex (1-100 nM), or 7t15-21s (1-100 nM). After 18 hrs, cells were treated in Brefeldin A (BFA) for 4 hrs. Cells were harvested and stained for surface markers CD3, CD4, CD16, CD56, CD19, CD8 and intracellular IFN-γ. After staining, cells were washed (1500 RPM for 5 minutes at room temperature) in FACS buffer (1× PBS (Hyclone) with 0.5% BSA (EMD Millipore) and 0.001% Sodium Azide (Sigma). After two washes, cells were analyzed by Flow Cytometry (Celesta-BD Bioscience). See FIGS. 20A and 20B.

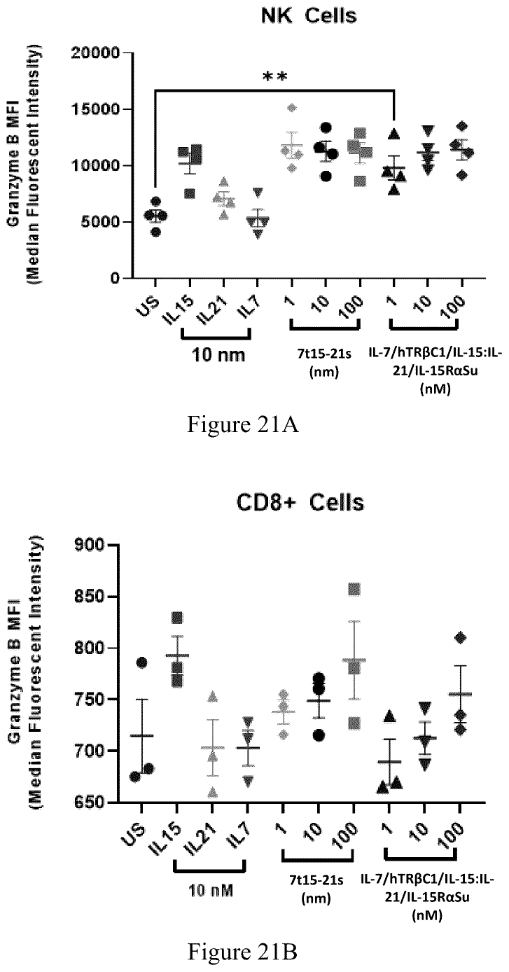

Additionally, fresh human leukocytes were obtained from healthy donors and PBMC were isolated. Cells were counted and resuspended in 2×10⁶/mL in 24 wells flat bottom plate in 2 mL of complete media (RPMI 1640 (Gibco) supplemented with 2 mM L-glutamine (Thermo Life Technologies), penicillin (Thermo Life Technologies), streptomycin (Thermo Life Technologies), and 10% FBS (Hyclone)). Cells were stimulated overnight with the following molecules: media alone, IL-7, IL-15 or IL-21, IL-7/hTRβC1/IL-15:hIL-21/IL-15RαSu complex (1-100 nM), or 7t15-21s (1-100 nM). After 18 hrs, cells were treated in Brefeldin A (BFA) for 4 hrs. Cells were harvested and stained for surface markers CD3, CD4, CD16, CD56, CD19, CD8 and intracellular granzyme-B. After staining, cells were washed (1500 RPM for 5 minutes at room temperature) in FACS buffer (1× PBS (Hyclone) with 0.5% BSA (EMD Millipore) and 0.001% Sodium Azide (Sigma)). After two washes, cells were analyzed by flow cytometry (Celesta-BD Bioscience). See FIGS. 21A and 21B.

Results show that after overnight stimulation, IFN-γ production was significantly higher by IL-7/hTRβC1/IL-15:IL-21/IL-15RαSu complex in both NK and CD8⁺ T cells compared to 7t15-21s, with granzyme-B expression significantly increased by IL-7/hTRβC1/IL-15:hIL-21/IL-15RαSu complex in NK cells and showed a trend toward increased expression in CD8⁺ T cells.

### Example 11: Increased Expression of IFN-γ and Granzyme-β in Human Lymphocytes

Fresh human leukocytes were obtained from healthy donors and PBMC were isolated. Cells were counted and resuspended in 2×10⁶/ml in 24 wells flat bottom plate in 2 mls of complete media (RPMI 1640 (Gibco) supplemented with 2 mM L-glutamine (Thermo Life Technologies), penicillin (Thermo Life Technologies), streptomycin (Thermo Life Technologies), and 10% FBS (Hyclone). Cells were stimulated overnight with the following molecules: media alone, IL-7, IL-15 or IL-21, or mixed cytokines of IL-7, IL-15 and IL-21 (10 nM), or IL-7/hTRβC1/IL-15:IL-21/IL-15RαSu complex (10 nM). After 18 hrs, cells were treated with Brefeldin A (BFA) for 4 hrs. Cells were harvested and stained for surface markers CD3, CD4, CD16, CD56, CD19, CD8 and intracellular IFN-γ. After staining, cells were washed (1500 RPM for 5 minutes at room temperature) in FACS buffer (1× PBS (Hyclone) with 0.5% BSA (EMD Millipore) and 0.001% Sodium Azide (Sigma)). After two washes, cells were analyzed by Flow Cytometry (Celesta-BD Bioscience).

Additionally, fresh human leukocytes were obtained from healthy donors and PBMC were isolated. Cells were counted and resuspended in 2×10⁶/mL in 24 wells flat bottom plate in 2 mls of complete media (RPMI 1640 (Gibco) supplemented with 2 mM L-glutamine (Thermo Life Technologies), penicillin (Thermo Life Technologies), streptomycin (Thermo Life Technologies), and 10% FBS (Hyclone). Cells were stimulated overnight with the following molecules: media alone, IL-7, IL-15 or IL-21, or mixed cytokines of IL-7, IL-15 and IL-21 (10 nM), or IL-7/hTRβC1/IL-15:hIL-21/IL-15RαSu complex (10 nM). After 18 hrs, cells were treated with Brefeldin-A (BFA) for 4 hrs. Cells were harvested and stained for surface markers CD3, CD4, CD16, CD56, CD19, CD8 and intracellular Granzyme-β. After staining, cells were washed (1500 RPM for 5 minutes at room temperature) in FACS buffer (1× PBS (Hyclone) with 0.5% BSA (EMD Millipore) and 0.001% Sodium Azide (Sigma)). After two washes, cells were analyzed by Flow Cytometry (Celesta-BD Bioscience).

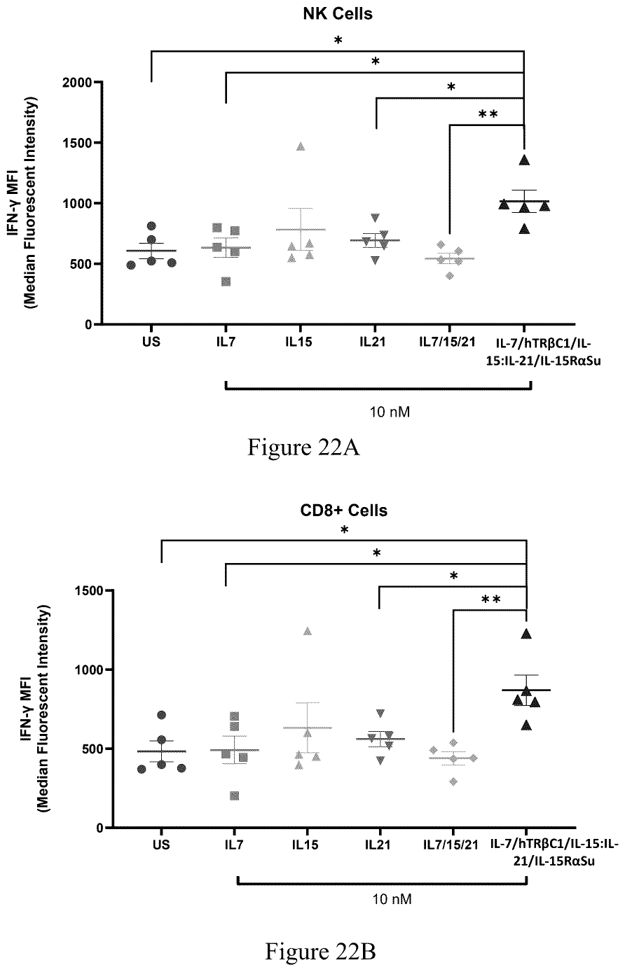

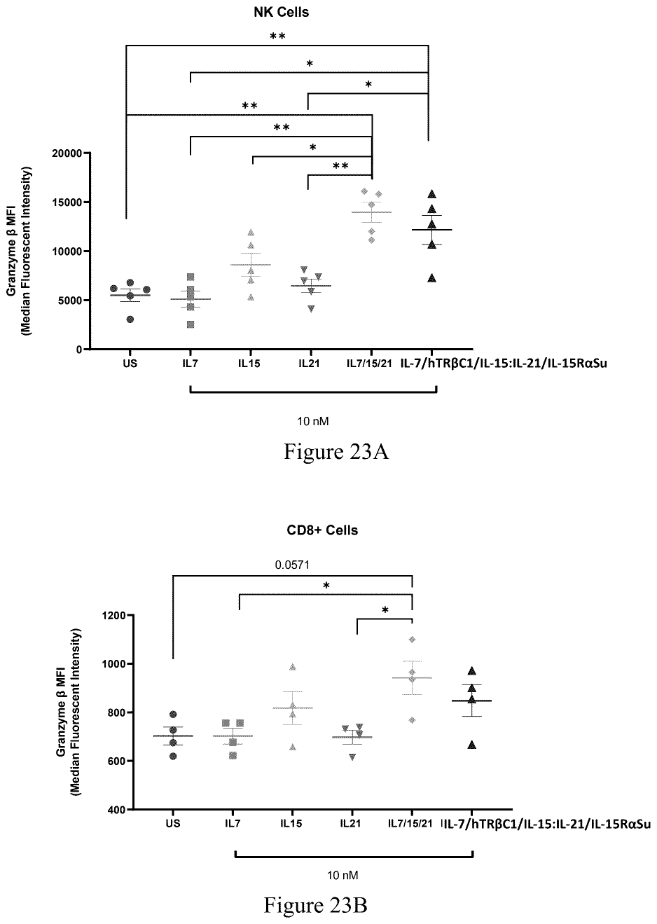

Results show that after overnight stimulation, IFN-7 production by both human NK cells and CD8⁺ T cells (FIGS. 22A and 22B) was significantly higher in response to IL-7/hTRβC1/IL-15:hIL-21/IL-15RαSu complex compared to the individual cytokines or cytokine mixture. Additionally, granzyme B (FIGS. 23A and 23B) expression in both human NK cells and CD8⁺ T cells was increased in a similar manner following treatment with either IL-7/hTRβC1/IL-15:hIL-21/IL-15RαSu complex or the cytokine mixture.

### Example 12: TGFβRII/hTRβC1/IL-15:TGFβRII/IL-15RαSu Complex Characterization

The nucleic acid and protein sequences of a TGFβRII/hTRβC1/IL-15:TGFβRII/IL-15RαSu complex are shown below.

The nucleic acid sequence of the TGFβRII/hTRβC1/IL-15:TGFβRII/IL-15RαSu complex is as follows:

(TGFR- hTRαC1-IL-15)
(SEQ ID NO: 191)
ATGGGCGTGAAGGTGCTGTTCGCCCTGATCTGCATCGCCGTGGCCGA
GGCCATTCCTCCTCACGTGCAGAAAAGCGTGAACAACGACATGATAG
TGACCGACAATAACGGCGCGGTCAAGTTCCCACAGCTCTGCAAATTT
TGCGACGTGAGATTTAGTACCTGCGACAATCAGAAATCCTGCATGAG
CAATTGTAGCATCACTAGCATCTGTGAAAAGCCCCAAGAGGTCTGCG
TAGCAGTGTGGAGAAAAAATGACGAGAACATCACACTAGAGACCGTG
TGCCATGACCCTAAGCTGCCTTATCATGATTTCATCCTTGAAGATGC
TGCTAGCCCTAAGTGCATTATGAAAGAGAAAAAGAAGCCTGGCGAAA
CCTTTTTCATGTGTAGCTGTAGTAGCGACGAGTGCAACGATAACATC
ATCTTCTCGGAGGAGTATAATACATCGAATCCTGATGGCGGCGGCGG
CAGCATCCCTCCTCATGTTCAGAAGAGCGTGAATAACGACATGATTG
TGACGGATAACAATGGCGCCGTAAAGTTCCCTCAGCTGTGTAAGTTC
TGCGATGTGCGATTTAGCACCTGCGACAATCAGAAGAGCTGTATGTC
CAATTGTTCAATCACAAGCATCTGTGAGAAGCCTCAAGAGGTGTGCG
TGGCCGTTTGGAGAAAGAACGACGAGAACATTACCCTTGAGACCGTA
TGCCACGATCCTAAGCTGCCGTATCACGACTTCATACTGGAGGATGC
CGCTAGCCCTAAATGCATCATGAAAGAAAAAAAAAAGCCTGGTGAGA
CATTTTTTATGTGTAGCTGCTCGTCTGACGAGTGCAATGACAATATC
ATTTTCAGCGAAGAATATAACACCTCAAACCCTGACGAGGACCTGAA
CAAGGTGTTCCCACCCGAGGTCGCTGTGTTTGAGCCATCAGAAGCAG
AGATCTCCCACACCCAAAAGGCCACACTGGTGTGCCTGGCCACAGGC
TTCTTCCCCGACCACGTGGAGCTGAGCTGGTGGGTGAATGGGAAGGA
GGTGCACAGTGGGGTCAGCACGGACCCGCAGCCCCTCAAGGAGCAGC
CCGCCCTCAATGACTCCAGATACTGCCTGAGCAGCCGCCTGAGGGTC
TCGGCCACCTTCTGGCAGAACCCCCGCAACCACTTCCGCTGTCAAGT
CCAGTTCTACGGGCTCTCGGAGAATGACGAGTGGACCCAGGATAGGG
CCAAACCCGTCACCCAGATCGTCAGCGCCGAGGCCTGGGGTAGAGCA
GACAACTGGGTGAACGTGATCAGCGACCTGAAGAAGATCGAGGACCT
GATTCAGAGCATGCACATCGACGCCACCCTGTACACCGAGAGCGACG
TGCACCCTAGCTGCAAGGTGACCGCCATGAAGTGCTTCCTGCTGGAG
CTGCAAGTGATCAGCCTGGAGAGCGGCGACGCTAGCATCCACGACAC
CGTGGAGAACCTGATCATCCTGGCCAACAACAGCCTGAGCAGCAACG
GCAACGTGACCGAGAGCGGCTGCAAGGAGTGCGAGGAGCTGGAGGAG
AAGAACATCAAGGAGTTCCTGCAGAGCTTCGTGCACATCGTGCAGAT
GTTCATCAACACAAGCTAATGA,
(TGFR-IL-15RαSushi)
(SEQ ID NO: 158)
ATGAAGTGGGTGACCTTCATCAGCCTGCTGTTCCTGTTCAGCAGCGC
CTACAGCATCCCGCCACACGTGCAAAAGAGCGTGAACAACGACATGA
TCGTTACGGACAACAATGGGGCCGTGAAGTTCCCTCAGTTATGCAAA
TTCTGTGACGTGAGATTCAGCACGTGCGACAATCAGAAGAGCTGCAT
GTCCAATTGCTCAATCACAAGCATCTGCGAGAAACCCCAAGAAGTTT
GCGTGGCCGTGTGGAGAAAGAATGACGAGAACATCACCCTGGAAACC
GTGTGCCATGATCCTAAACTCCCTTACCATGACTTTATCTTGGAGGA
TGCTGCTAGCCCAAAGTGCATCATGAAAGAGAAGAAGAAACCGGGGG
AAACTTTTTTCATGTGTTCATGCAGCTCTGACGAGTGTAATGATAAC
ATTATCTTCAGCGAAGAGTATAACACTAGCAATCCTGATGGAGGTGG
CGGATCTATCCCCCCCCATGTGCAAAAATCAGTGAACAATGACATGA
TCGTGACCGATAATAACGGGGCGGTAAAGTTTCCTCAACTGTGTAAA
TTTTGCGACGTCAGATTCAGCACATGCGACAATCAAAAGTCCTGCAT
GAGCAACTGCAGCATCACCTCTATCTGTGAGAAACCACAAGAAGTAT
GTGTGGCGGTATGGCGGAAGAACGATGAAAATATCACGCTGGAGACC
GTTTGCCATGATCCTAAGCTGCCATATCACGATTTCATCCTGGAGGA
CGCCGCTAGCCCTAAGTGTATCATGAAGGAGAAGAAGAAGCCCGGTG
AGACCTTTTTCATGTGCTCATGCAGCTCCGACGAGTGCAACGACAAC
ATCATCTTCAGCGAGGAGTACAATACAAGCAATCCTGACATCACCTG
CCCTCCTCCTATGAGCGTGGAGCACGCCGACATCTGGGTGAAGAGCT
ACAGCCTGTACAGCAGAGAGAGATACATCTGCAACAGCGGCTTCAAG
AGAAAGGCCGGCACAAGCAGCCTGACCGAGTGCGTGCTGAACAAGGC
CACCAACGTGGCCCACTGGACCACCCCTAGCCTGAAGTGCATCAGAT
AA.

The amino acid sequence of the TGFβRII/hTRβC1/IL-15:TGFβRII/IL-15RαSu complex is as follows:

(TGFR-hTRβC1-IL-15)
(SEQ ID NO: 193)
MGVKVLFALICIAVAEAIPPHVQKSVNNDMIVTDNNGAVKFPQLCKFCDVRFSTCDNQKSCMSN
CSITSICEKPQEVCVAVWRKNDENITLETVCHDPKLPYHDFILEDAASPKCIMKEKKKPGETFF
MCSCSSDECNDNIIFSEEYNTSNPDGGGGSIPPHVQKSVNNDMIVTDNNGAVKFPQLCKFCDVR
FSTCDNQKSCMSNCSITSICEKPQEVCVAVWRKNDENITLETVCHDPKLPYHDFILEDAASPKC
IMKEKKKPGETFFMCSCSSDECNDNIIFSEEYNTSNPDEDLNKVFPPEVAVFEPSEAEISHTQK
ATLVCLATGFFPDHVELSWWVNGKEVHSGVSTDPQPLKEQPALNDSRYCLSSRLRVSATFWQNP
RNHFRCQVQFYGLSENDEWTQDRAKPVTQIVSAEAWGRADNWVNVISDLKKIEDLIQSMHIDAT
LYTESDVHPSCKVTAMKCFLLELQVISLESGDASIHDTVENLIILANNSLSSNGNVTESGCKEC
EELEEKNIKEFLQSFVHIVQMFINTS,
(TGFR-IL-15RαSushi)
(SEQ ID NO: 194)
MKWVTFISLLFLFSSAYSIPPHVQKSVNNDMIVTDNNGAVKFPQLCKFCDVRFSTCDNQKSCMS
NCSITSICEKPQEVCVAVWRKNDENITLETVCHDPKLPYHDFILEDAASPKCIMKEKKKPGETF
FMCSCSSDECNDNIIFSEEYNTSNPDGGGGSIPPHVQKSVNNDMIVTDNNGAVKFPQLCKFCDV
RFSTCDNQKSCMSNCSITSICEKPQEVCVAVWRKNDENITLETVCHDPKLPYHDFILEDAASPK
CIMKEKKKPGETFFMCSCSSDECNDNIIFSEEYNTSNPDITCPPPMSVEHADIWVKSYSLYSRE
RYICNSGFKRKAGTSSLTECVLNKATNVAHWTTPSLKCIR.

To determine the purity and molecular weight of the TGFβRII/hTRβC1/IL-15:TGFβRII/IL-15RαSu complex, a purified protein sample was analyzed by sodium dodecyl sulfate polyacrylamide gel (4-12% Bis-Tris gel) electrophoresis (SDS-PAGE) method under reduced condition (1× MOPS running buffer at 150V for 60 min). After electrophoresis, the gel was stained with InstantBlue for about 35 min, followed by destaining overnight in purified water.

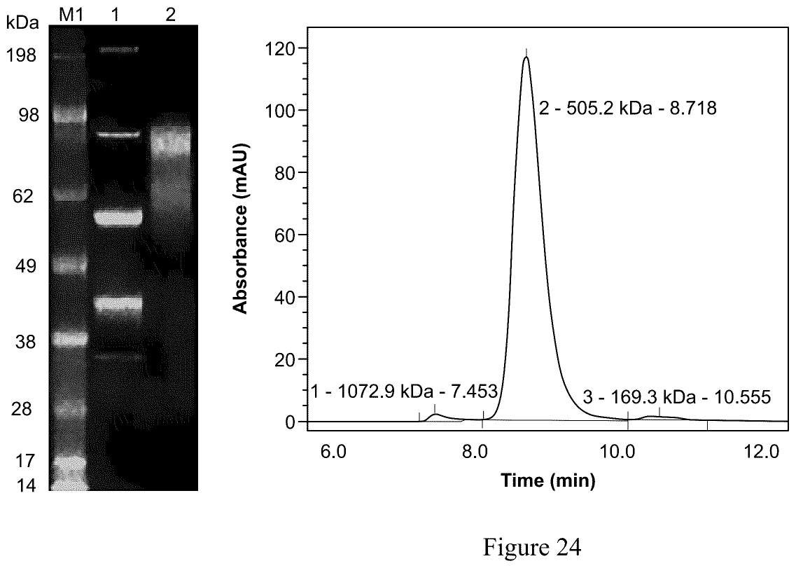

Results shows that the TGFβRII/hTRβC1/IL-15 subunit is 59kD and TGFβRII/IL-15RαSu subunit is 38 kDa based on protein sequence, which is consistent with unit sizes shown in lane-1 in the reduced SDS-PAGE after deglycosylation (FIG. 24, left). Lane-2 shows that the two subunits without deglycosylation treatment are much larger than that in lane-1, which indicates that TGFβRII/hTRβC1/IL-15:TGFβRII/IL-15RαSu complex is highly glycosylated.

However, TGFβRII/hTRβC1/IL-15:TGFβRII/IL-15RαSu complex displays 505 kDa based on size exclusion chromatography analysis (FIG. 24, right), which suggests that TGFβRII/hTRβC1/IL-15:TGFβRII/IL-15RαSu complex naturally forms a trimer or is an irregularly shaped protein.

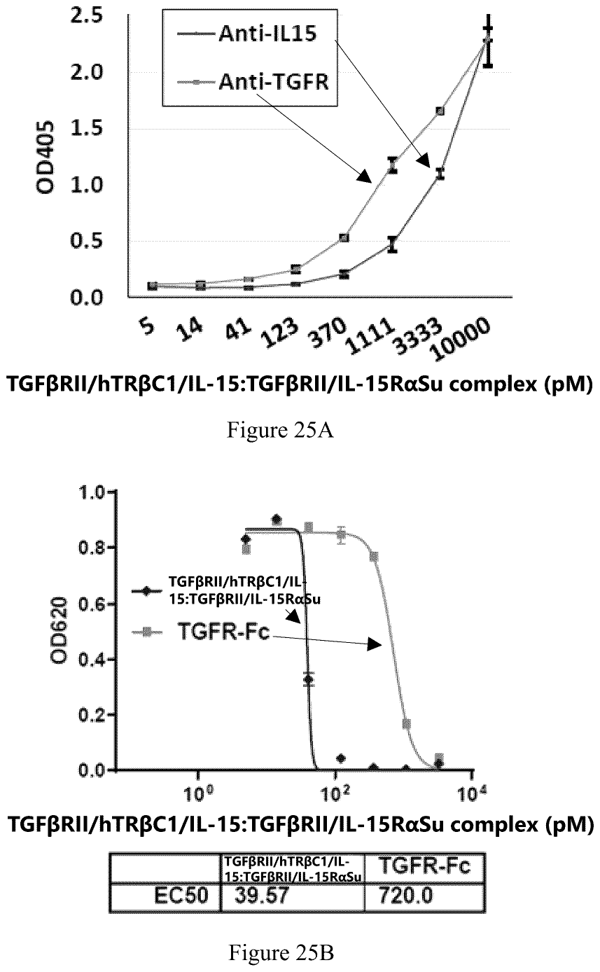

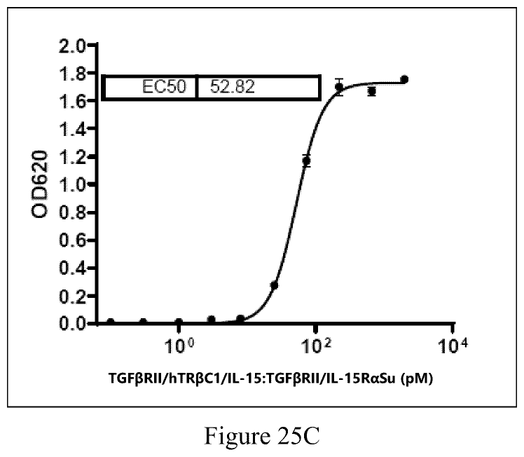

For component characterization of TGFβRII/hTRβC1/IL-15:TGFβRII/IL-15RαSu complex, TGFβRII/hTRβC1/IL-15:TGFβRII/IL-15RαSu complex was captured by anti-hTRBC1 antibody and detected by biotinylated anti-IL-15 or anti-TGFβR antibody in an ELISA. IL-15 and TGFb1 trap activities were respectively determined by HEK Blue IL-15 reporter cell line and HEK-Blue TGF-b reporter cell line. Results show that IL-15 and TGFβR are detected by correspondent antibodies with ELISA, wherein biological activities were demonstrated with their reporter cell lines (FIGS. 25A-25C).

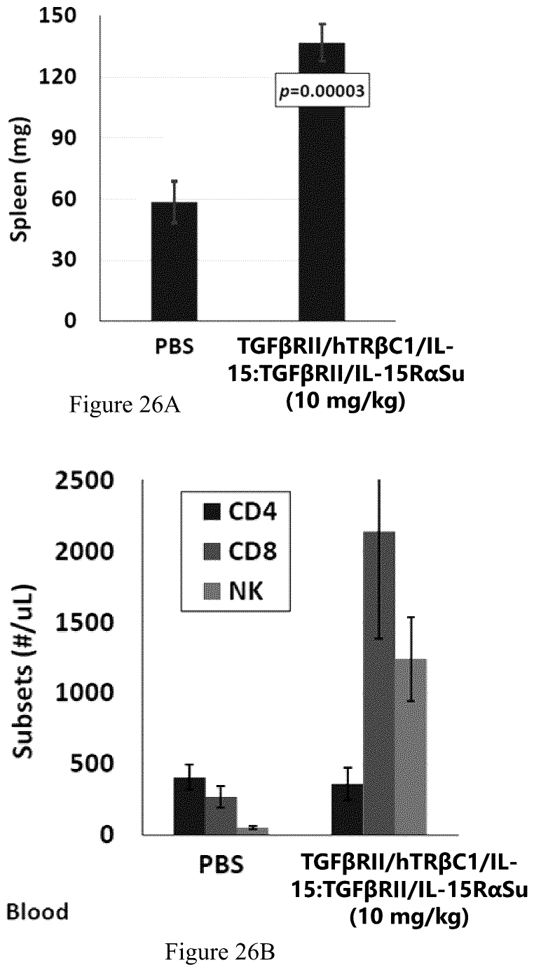

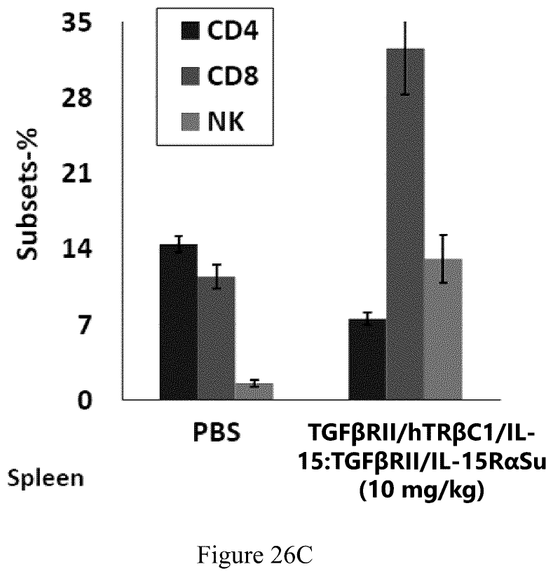

Additionally, immunostimulatory activity of TGFβRII/hTRβC1/IL-15:TGFβRII/IL-15RαSu complex was analyzed in C57BL/6 mice. C57BL/6 mice (n=4) were treated (s.c.) with TGFβRII/hTRβC1/IL-15:TGFβRII/L-15RαSu complex at 10 mg/kg. The mouse spleen weights were measured, and the lymphocyte subsets in blood and spleen were determined by flow cytometry 3 days later. Results show that TGFβRII/hTRβC1/IL-15:TGFβRII/IL-15RαSu complex significantly induced CD8⁺ T cells and NK cells to proliferate (p<0.01) in blood and spleen (FIGS. 26A-26C).

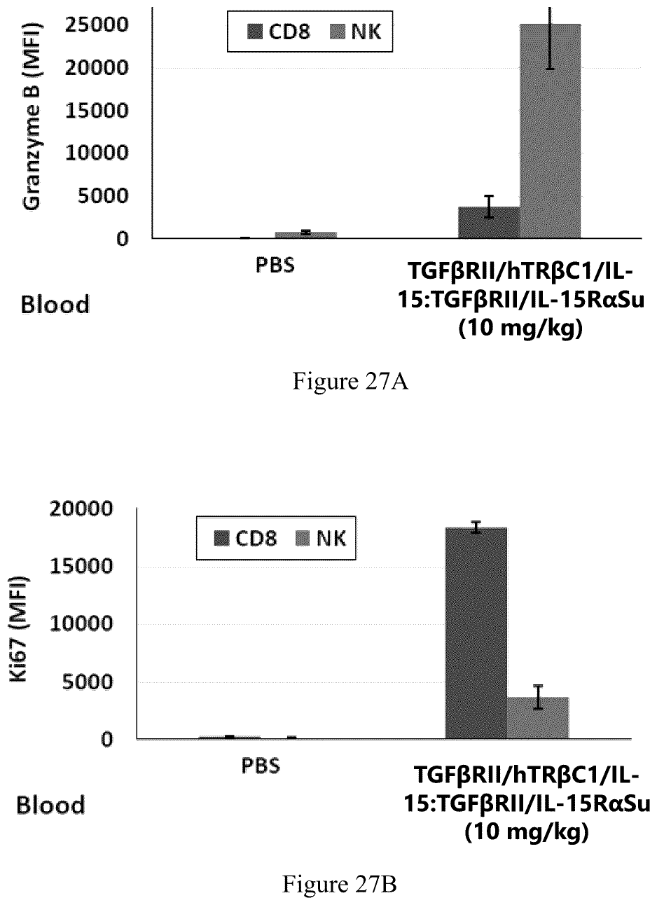

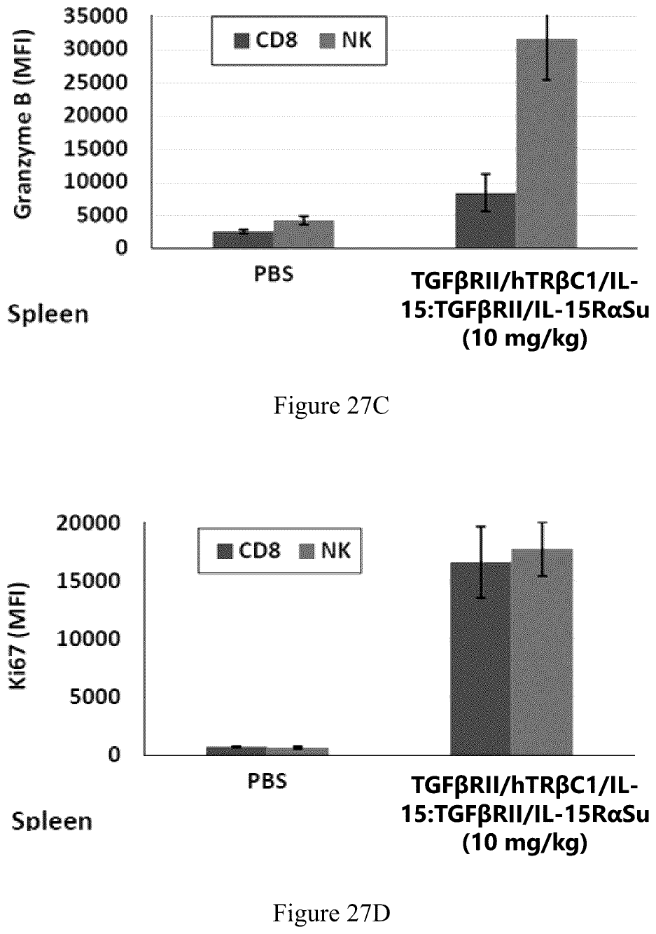

Furthermore, C57BL/6 mice (n=4) were treated (s.c.) with TGFβRII/hTRβC1/IL-15:TGFβRII/IL-15RαSu complex at 10 mg/kg. The expression of granzyme B and Ki67 by CD8⁺ T cells and NK cells in blood and spleen were determined by flow cytometry 3 days later. Results showed that TGFβRII/hTRβC1/IL-15:TGFβRII/IL-15RαSu complex induced CD8⁺ T cells and NK cells to express higher cytotoxic molecule granzyme B and proliferation marker Ki67 (p<0.01) (FIGS. 27A-27D).

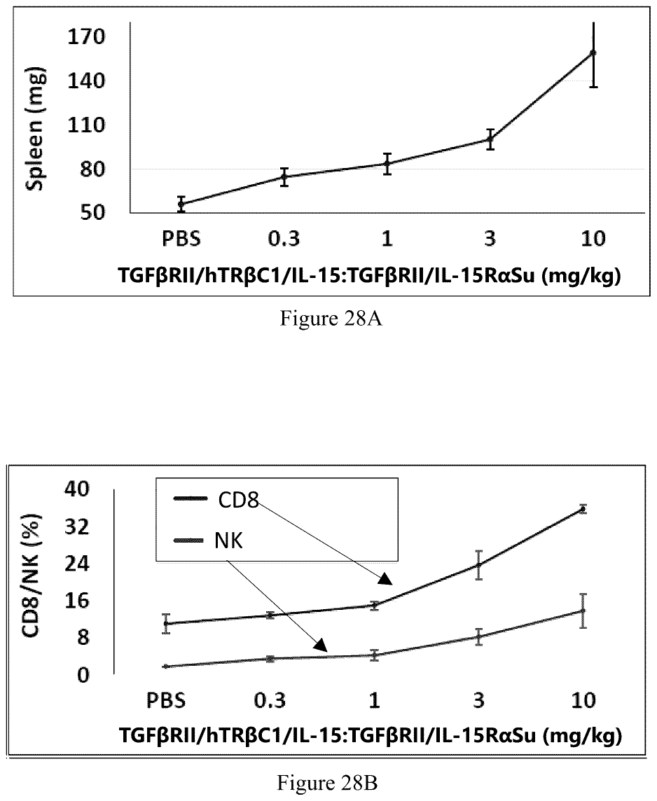

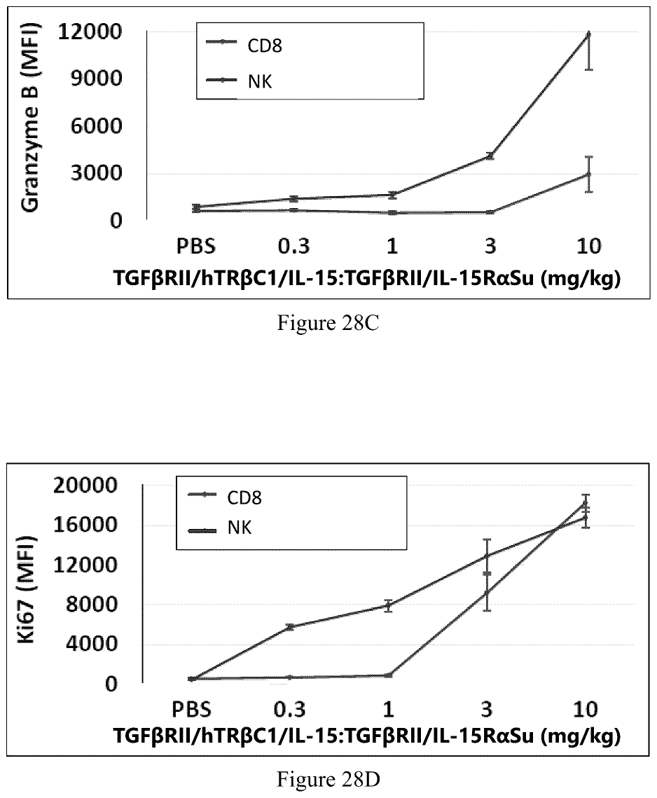

Finally, C57BL/6 mice (n=3) were treated (s.c.) with TGFβRII/hTRβC1/IL-15:TGFβRII/IL-15RαSu complex. The expression of granzyme B and Ki67 by CD8⁺ T cells and NK cells in spleen were determined by flow cytometry 3 days later. Results also showed that TGFβRII/hTRβC1/IL-15:TGFβRII/IL-15RαSu complex significantly induced CD8⁺ T cells and NK cells to proliferate, express granzyme B and Ki67 in a dose dependent manner (FIGS. 28A-28D).

### Example 13: Titration of TGFβRII/hTRβC1/IL-15: αTF scFv/IL-15RαSu/αCD3 scFv Complex on AsPC1 and MIA Pancreatic Cancer Cell Lines

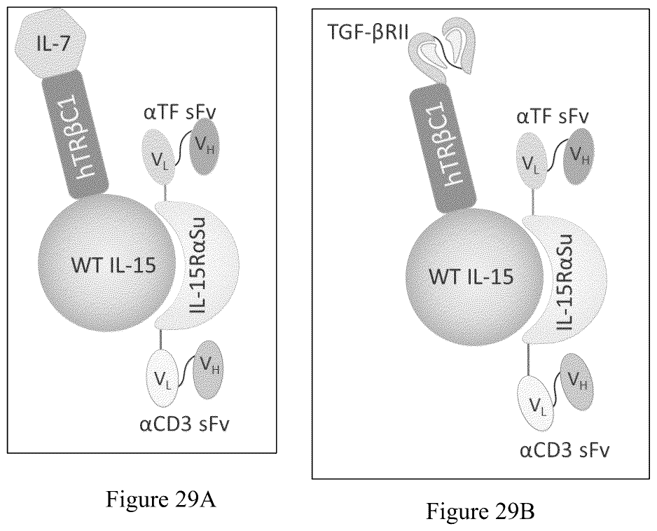

AsPC1 (TF-positive) or MIA-PaCa-2 (TF-negative) cells (12,000 cells/well) were cultured in a 96-well flat-bottom plate for 24 hours to determine optimal drug concentration to avoid non-specific interactions, with a focus on evaluating T-cell activation at various concentrations. PBMCs were then added at a 10:1 effector-to-target ratio, followed by treatment with TGFβRII/hTRβC1/IL-15: αTF scFv/IL-15RαSu/αC3 scFv complex (FIG. 29B; see also Example 31) at concentrations of 100 nM, 10 nM, 1 nM, and 0.1 nM. After 20 hours, effector cells were collected, stained with Live-Dead, anti-CD3, anti-CD8, anti-CD4, anti-CD25, and anti-CD69, and analyzed by flow cytometry on a FACSCalibur to assess CD25+ and CD69+ expression within CD8+ and CD4⁺ T cell populations.

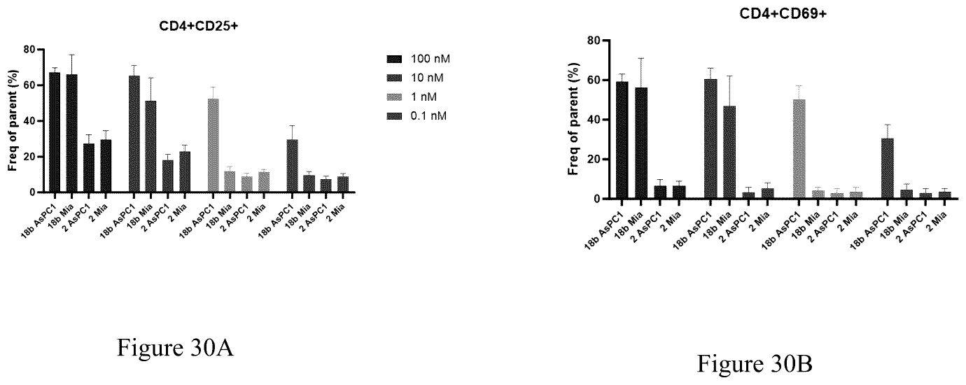

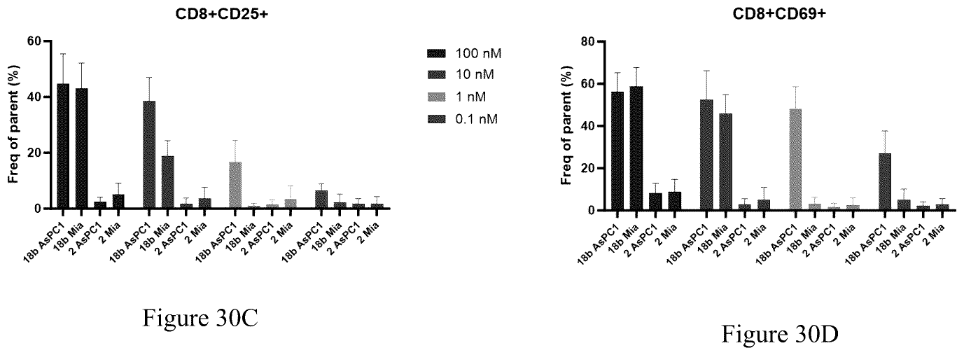

Results showed that at a concentration of 1 nM, T-cell activation, marked by increased CD25 and CD69 expression, was observed in both CD8+ and CD4⁺ T cell populations only with TF-positive AsPC1 cells, showing a significant difference compared to MIA cells. This indicates that 1 nM is the optimal concentration for specific T-cell engager activity. Higher concentrations (100 nM and 10 nM) resulted in non-specific T cell activation when incubated with the MIA cell line, which lacks tissue factor. See FIGS. 30A-D.

### Example 14: TGFβRII/hTRβC1/IL-15: αTF scFv/IL-15RαSu/αCD3 scFv Complex-Mediated Target Cell-T Cell Conjugation and Cytotoxicity

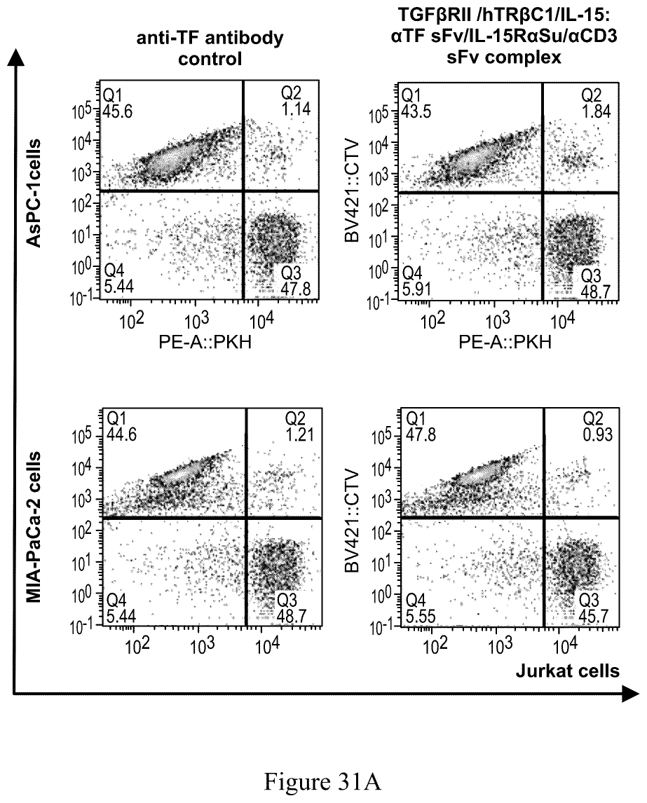

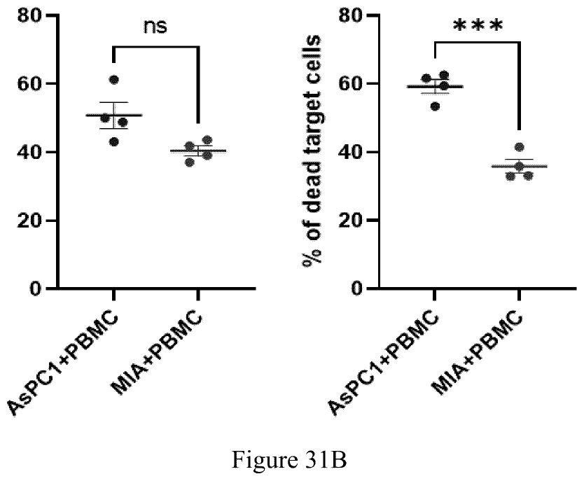

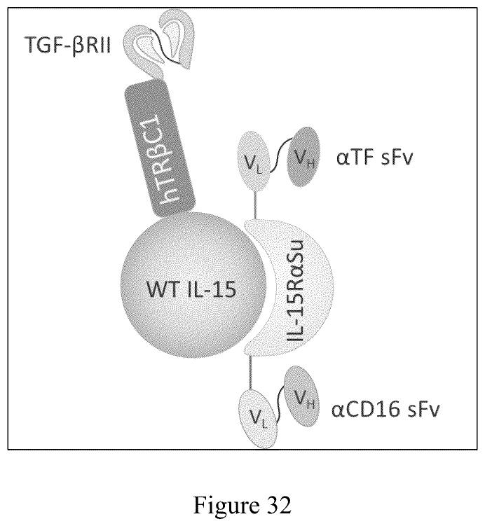

To analyze cell-cell conjugation, CellTrace Violet kit (Invitrogen) labeled AsPC-1 (TF⁺) or MIA-Pa-Ca-2 (TF⁻) cells (1×10⁵ cells, 50 μL each) were mixed with 50 μL of Mini26 kit (Millipore-Sigma) labeled CD3⁺ Jurkat cells (1×10⁵ cells) in the presence or absence of TGFβRII/hTRβC1/IL-15: αTF scFv/IL-15RαSu/αC3 scFv complex (TCE) (see Example 31 for construct) or HCW9101 (anti-TF antibody control) (20 pg each). After incubation at 37° C., 5% CO₂ for 30 min, the mixtures were analyzed on FACSCelestra. Crosslinking of CD3+ Jurkat cells and TF⁺ AsPC-1 or TP MIA-Pa-Ca-2 cells by TCEs was evaluated through the appearance of Mini26+/CellTrace Violet⁺ cell-cell conjugates (FIG. 31A). The results show equivalent cell crossing between Jurkat cells and TF+ AsPC-1 or TF⁻ MIA-Pa-Ca-2 cells using the anti TF Ab, whereas increased crossing between Jurkat cells and TF⁺ AsPC-1 cells compared to TF⁻ MIA-Pa-Ca-2 cells was seen with the TGFβRII/hTRβC1/IL-15: αTF scFv/IL-15RαSu/αC3 scFv complex.

Additionally, to determine T cell-mediated cytotoxicity, CellTrace Violet kit (Invitrogen) labeled AsPC-1 (TF⁺) or MIA-Pa-Ca-2 (TF⁻) cells (3×10⁴ cells/well) were mixed with PBMCs from 4 donors (3×10⁵ cells/well) in the presence of TGFβRII/hTRβC1/IL-15: αTF scFv/IL-15RαSu/αC3 scFv complex or an anti-human Tissue Factor antibody of the human IgG1 isotype (100 nM each) respectively, in a V-shape 96-well plate (Corning). After 20 h incubation, the cell mix was spun down and resuspend in complete-medium with Propidium iodide (PI, ex/em=488/610) at 2 μg/mL and analyzed with FACSCelestra. The percentage of target cell killing was evaluated through the appearance of PI+ and CellTrace Violet+dead cells (FIG. 31B). The results show equivalent cytotoxicity of PBMCs against TF⁺ AsPC-1 or TF⁻ MIA-Pa-Ca-2 cells using the anti TF Ab, whereas increased cytotoxicity of PBMCs against TF⁺ AsPC-1 cells compared to TP MIA-Pa-Ca-2 cells was seen with the TGFβRII/hTRβC1/IL-15: αTF scFv/IL-15RαSu/αC3 scFv complex.

### Example 15: Anti-Tumor Efficacy of TGFβRII/hTRβC1/IL-15:TGFβRII/IL-15RαSu Complex in a Melanoma Mouse Tumor Model

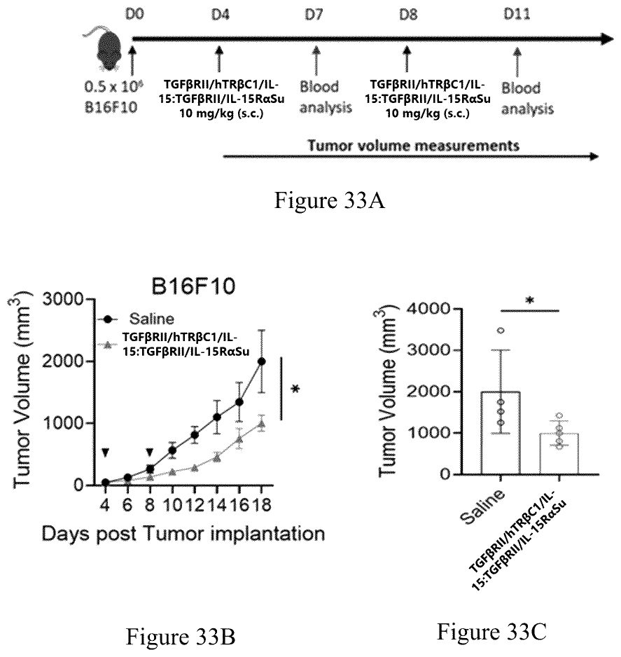

To determine whether TGFβRII/hTRβC1/IL-15:TGFβRII/IL-15RαSu complex (see Example 12) exhibits anti-tumor efficacy in a B16F10 mouse tumor model, six-week old C57BL6/j mice (n=6-7) were injected subcutaneously with B16F10 cells (0.5×10⁶). Once tumor volumes reached 50-100 mm³, one group of mice received TGFβRII/hTRβC1/IL-15:TGFβRII/IL-15RαSu complex (10 mg/kg, Day 4 and Day 8 post tumor implantation). Tumor volumes were measured every 2 days. FIG. 33A shows a schema of the experimental study. FIG. 33B shows tumor volumes at days indicated comparing saline treated and TGFβRII/hTRβC1/IL-15:TGFβRII/IL-15RαSu complex treated B16F10 tumor-bearing mice. Arrow heads indicate treatment time. FIG. 33C shows tumor volumes on Day 18 (end of study) between the saline and TGFβRII/hTRβC1/IL-15:TGFβRII/IL-15RαSu complex groups. Statistical comparison was performed using Student's t test. FIGS. 33B and 33C show that TGFβRII/hTRβC1/IL-15:TGFβRII/IL-15RαSu complex exhibits anti-tumor efficacy in B16F10 mouse tumor model.

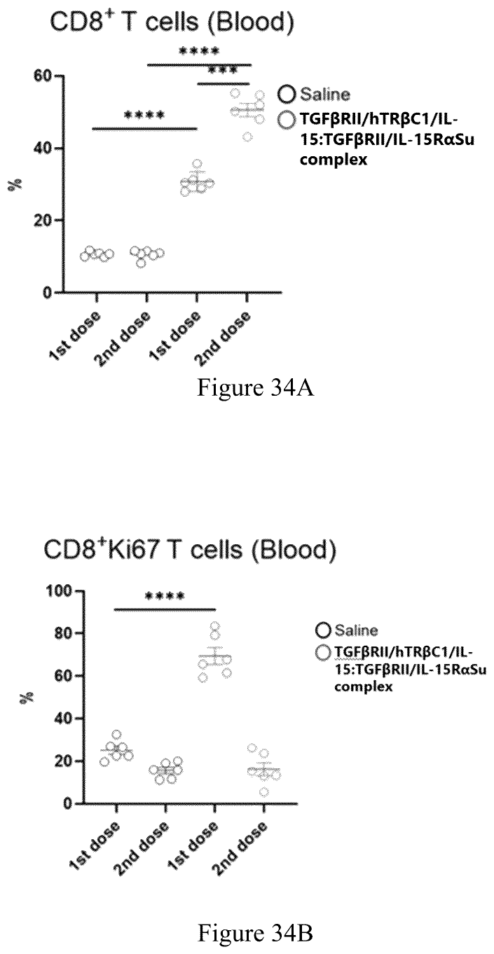

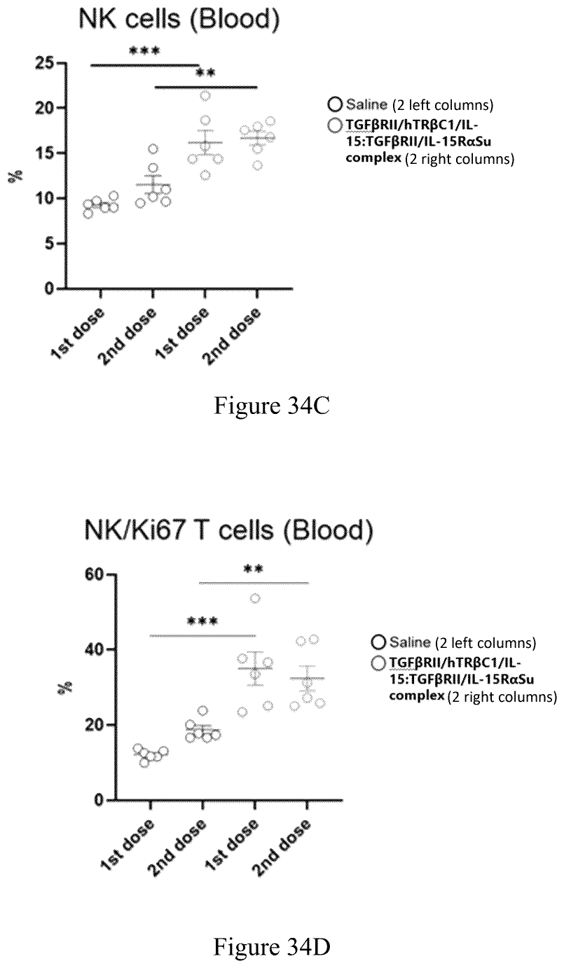

Additionally, to determine whether TGFβRII/hTRβC1/IL-15:TGFβRII/IL-15RαSu complex expands proliferating CD8⁺ T cells and NK cells in peripheral blood of B16F10 tumor-bearing mice, six-week old C57BL6/j mice (n=6-7) were injected subcutaneously with B16F10 cells (0.5×10⁶). Once tumor volumes reached 50-100 mm³, one group of mice received TGFβRII/hTRβC1/IL-15:TGFβRII/IL-15RαSu complex (10 mg/kg, Day 4 and Day 8 post tumor implantation). Tumor volumes were measured every 2 days. Blood was collected on day 3 post treatment and analyzed by flow cytometry. FIGS. 34A and 34B show frequencies of total CD8⁺ T cells and Ki67⁺ proliferating CD8⁺ T cells, respectively, in peripheral blood of saline-treated and TGFβRII/hTRβC1/IL-15:TGFβRII/IL-15RαSu complex treated mice on day 3 post treatment. FIGS. 34C and 34D show that frequencies of total NK1.1 and Ki67⁺ proliferating NK cells, respectively, in peripheral blood of saline-treated and TGFβRII/hTRβC1/IL-15:TGFβRII/IL-15RαSu complex treated mice on day 3 post treatment. Statistical comparison between 2 groups was performed using Student's t test. FIGS. 34A-34D show TGFβRII/hTRβC1/IL-15:TGFβRII/IL-15RαSu complex expands proliferating CD8+ T cells and NK cells in peripheral blood of B16F10 tumor-bearing mice.

### Example 16: IL-7/hTRβC1/IL-15:hIL-21/IL-15RαSu Complex Reduces Tumor Size of EG-7OVA Tumors and B16F10 Melanoma Tumors in C57BL/6 Mice

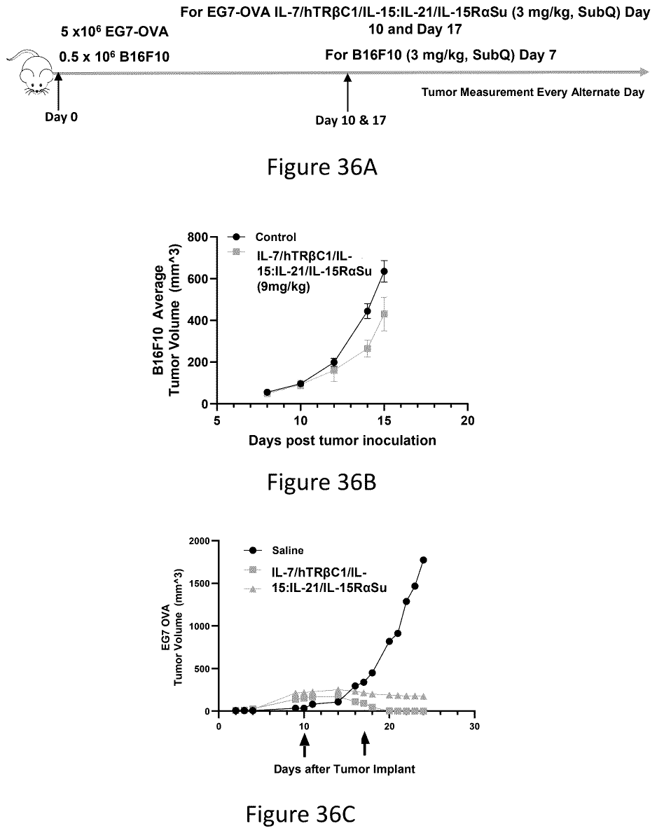

The EG7-OVA tumor cell line is a mouse lymphoma (EL4) cell line that has been transfected with a plasmid expressing chicken ovalbumin (OVA). B16F10 is a melanoma tumor cell line. FIG. 36A shows a schema of the experimental study.

Compared to the control, IL-7/hTRβC1/IL-15:IL-21/IL-15RαSu complex treatment showed a reduction in tumor volumes in both B16F10 and EG7-OVA tumor models in the C57BL/6 mouse model. In some cases, the treatment even led to complete tumor clearance in the pilot experiment. See FIGS. 36B-C.

### Example 17: IL-7/hTRβC1/IL-15:hIL-21/IL-15RαSu Complex Activate Common γ-Chain Cytokine Signaling Pathways Via STAT3 and STAT5

PBMCs derived from health human donors were thawed and then stimulated with either anti-media alone or IL-7/hTRβC1/IL-15:IL-21/IL-15RαSu complex (100 nM) for 30 minutes at 37° C. Paraformaldehyde solution (1.6%) was added to each well and incubated at room temperature for 10 min. Cells were washed with FACS buffer and centrifuged at 1300 RPM for 5 minutes. The supernatant was removed, and chilled methanol (100 μL) was added to each well to permeabilize the cells for 30 min at 4° C. The cells were then washed with FACS buffer and resuspended in pSTAT5-Alexa Fluor 647 or pSTAT3-FITC antibody (BioLegend). Cells were washed with FACS buffer before flow cytometric analysis.

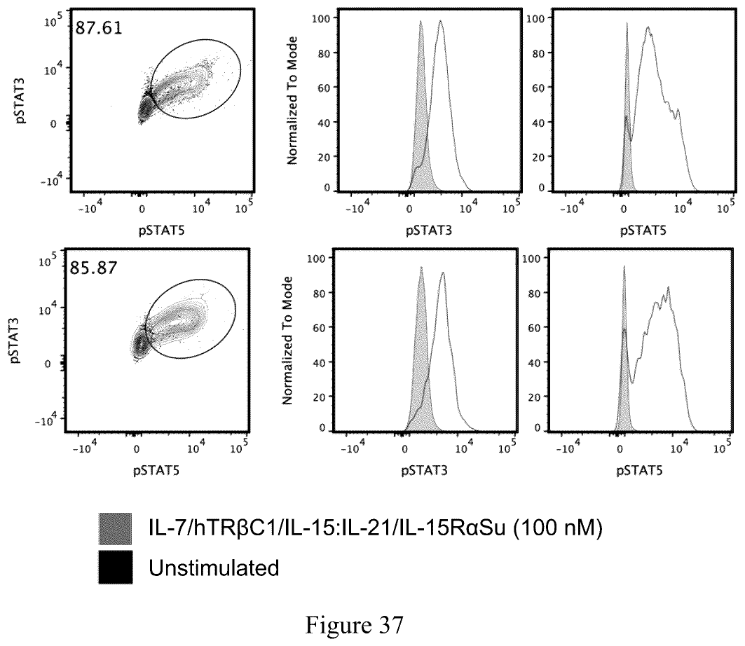

Results showed IL-7/hTRβC1/IL-15:IL-21/IL-15RαSu complex activated pSTAT3 and pSTAT5 signaling pathways in human PBMCs, which will direct NK and CD8⁺ cell survival and differentiation (FIG. 37).

### Example 18: IL-7/hTRβC1/IL-15:hIL-21/IL-15RαSu Complex Enhances IFN-γ Production in Immune Cells in the Presence of Immune Checkpoint Inhibitors

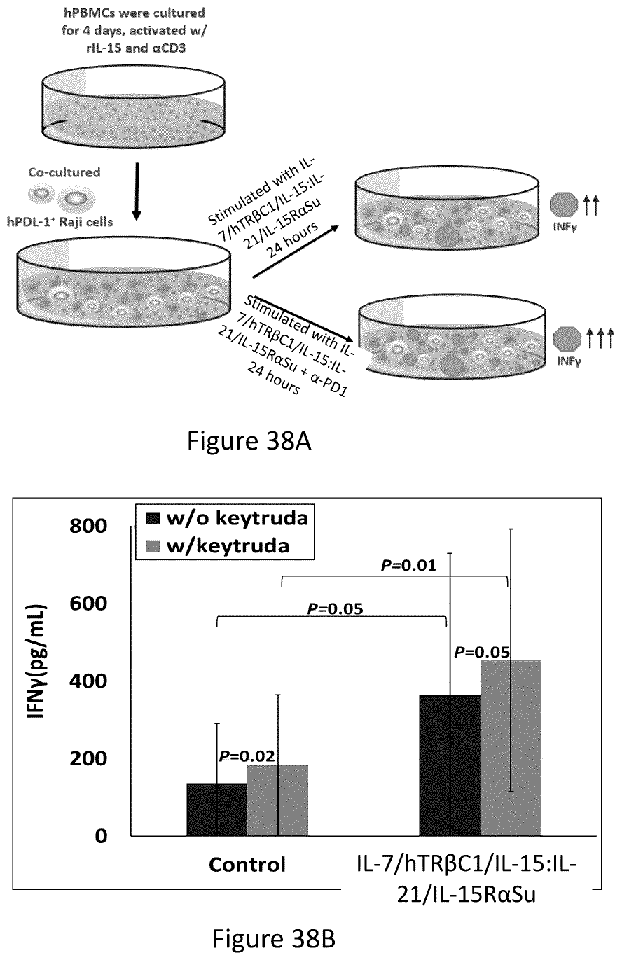

See FIG. 38A for a schematic of the assay performed. Activated human PBMCs were co-cultured with hPD-L1+ Raji tumor cells followed by treatment with IL-7/hTRβC1/IL-15:IL-21/IL-15RαSu alone or in combination with anti-PD-1 Ab (Keytruda).

IL-7/hTRβC1/IL-15:IL-21/IL-15RαSu complex significantly increased IFNγ secretion by human PBMCs in the presence of α-PD-1, demonstrating a synergistic enhancement of immune activation. Notably, IL-7/hTRβC1/IL-15:IL-21/IL-15RαSu complex alone also elevated IFNγ levels by human PBMCs compared to control, indicating its independent immunostimulatory effect and potential to complement α-PD-1 in combination therapy. See FIG. 38B.

### Example 19: IL-7/hTRβC1/IL-15:hIL-21/IL-15RαSu Complex Reduces Tumor Size of EG7-OVA Tumor in C57BL/6 Mice in the Presence of Immune Checkpoint Inhibitors α-PD-1

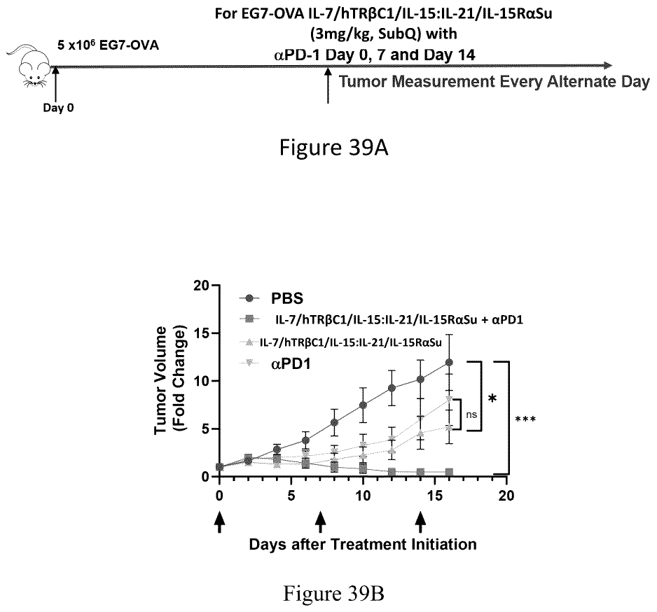

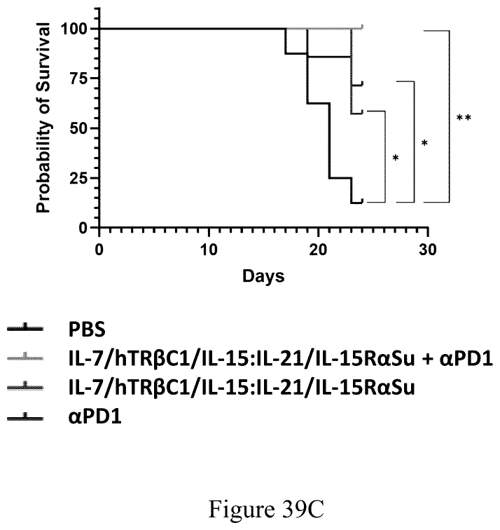

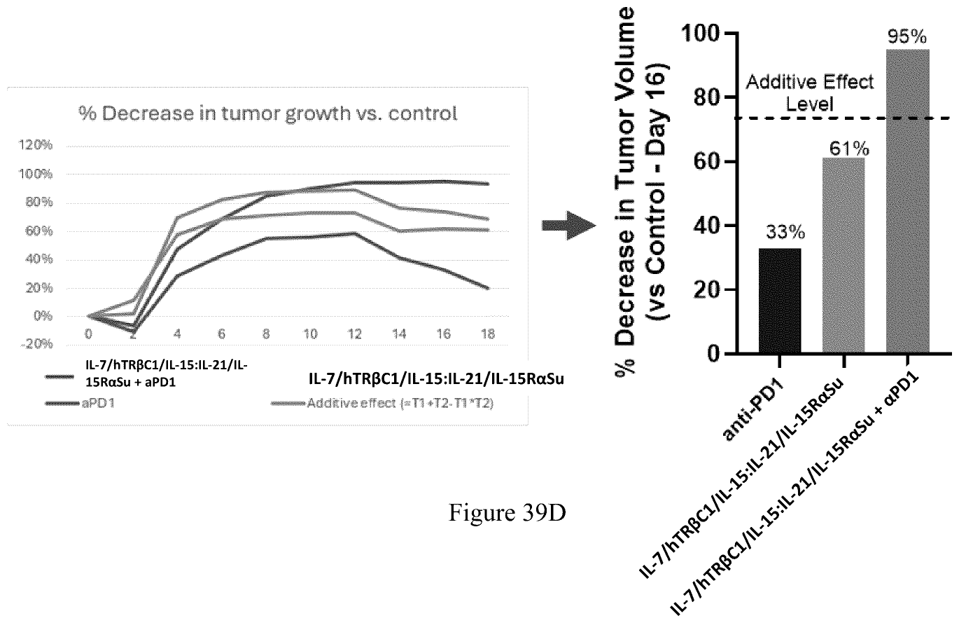

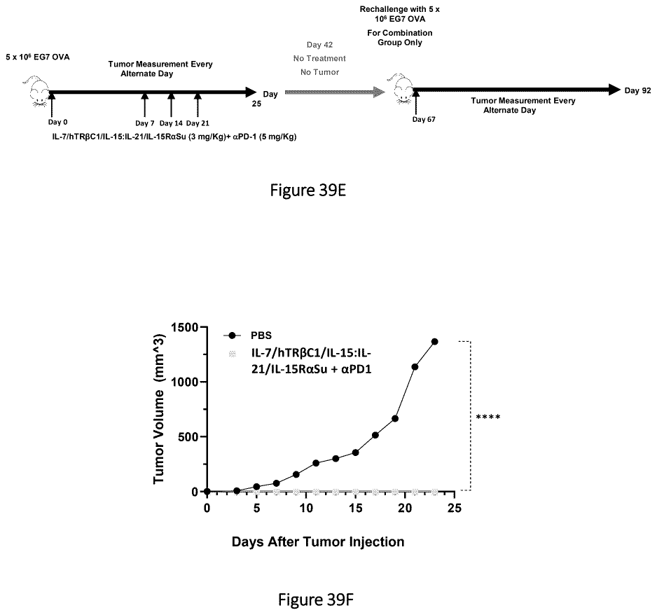

To evaluate IL-7/hTRβC1/IL-15:IL-21/IL-15RαSu complex antitumor efficacy, 5×10⁵ mouse EG7-OVA T lymphoblast cells (CRL-2113, ATCC) were subcutaneously injected into C57BL/6 mice (day 0) followed by subcutaneous injection of PBS or IL-7/hTRβC1/IL-15:IL-21/IL-15RαSu complex (3 mg/kg) or αPD-1 200 μg or IL-7/hTRβC1/IL-15:IL-21/IL-15RαSu complex fusion protein with α-PD-1 on day 8 after tumor implantation. FIG. 39A shows a schema of the experimental study.

Tumor growth in all studies was monitored by caliper measurement, and tumor volume was calculated using the formula V=(L×W2)/2, where L is the largest tumor diameter and W is the perpendicular tumor diameter. Tumor volume was measured every other day and mice with tumors ≥4000 mm³ were sacrificed per IACUC regulation (FIGS. 39B and 39D). Mouse survival was also assessed throughout the study period (FIG. 39C).

Treatment with IL-7/hTRβC1/IL-15:IL-21/IL-15RαSu complex alone and in combination with anti-PD-1 (αPD-1) significantly reduced EG7-OVA tumor growth in C57BL/6 mice compared to controls. Additionally, IL-7/hTRβC1/IL-15:IL-21/IL-15RαSu complex with αPD-1 showed synergistic effects to control tumor growth in C57BL/6 mice. It was also observed that tumor-bearing mice treated with IL-7/hTRβC1/IL-15:IL-21/IL-15RαSu complex in combination with αPD-1 survived longer.

Further, the mice group previously challenged with EG7-OVA tumor and treated with IL-7/hTRβC1/IL-15:IL-21/IL-15RαSu complex with α-PD-1 mAb were rechallenged with EG7-OVA tumor and compared with aged-match control saline treated mice. No tumors developed in group which were treated previously with IL-7/hTRβC1/IL-15:IL-21/IL-15RαSu complex with αPD-1 while tumor growth continued in saline treated mice. This experiment indicates that a memory immune response was established by the combination therapy. (FIGS. 39E-39F.)

The observed anti-tumor efficacy suggests that IL-7/hTRβC1/IL-15:IL-21/IL-15RαSu complex may offer a potent alternative to existing cytokine combination therapies, such as IL-15 plus αPD-1. Furthermore, the data indicate that the combination treatment significantly extended the survival of the treated mice. IL-7/hTRβC1/IL-15:IL-21/IL-15RαSu complex could be efficacious in combination with immune-check point inhibitors in patients with relapsed/refractory NSCLC.

### Example 20: Characterization of TGFβRII/hTRβC1/IL-15:Diabody Keytruda/IL-15RαSu/IL-7

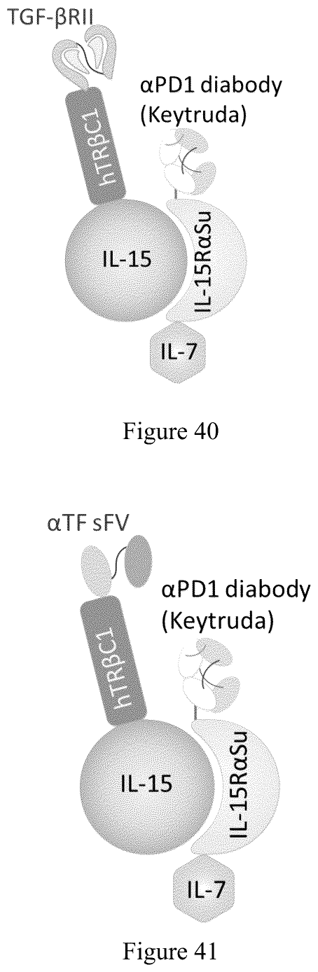

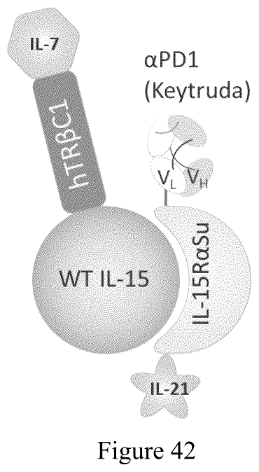

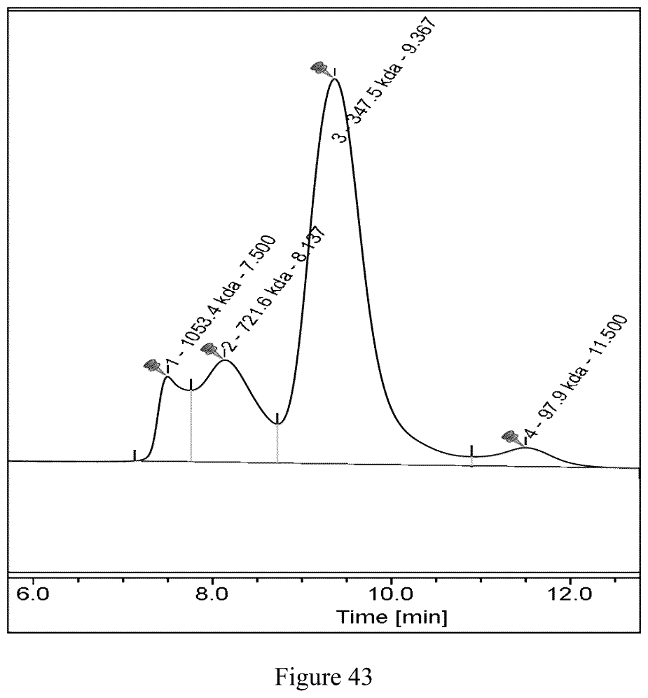

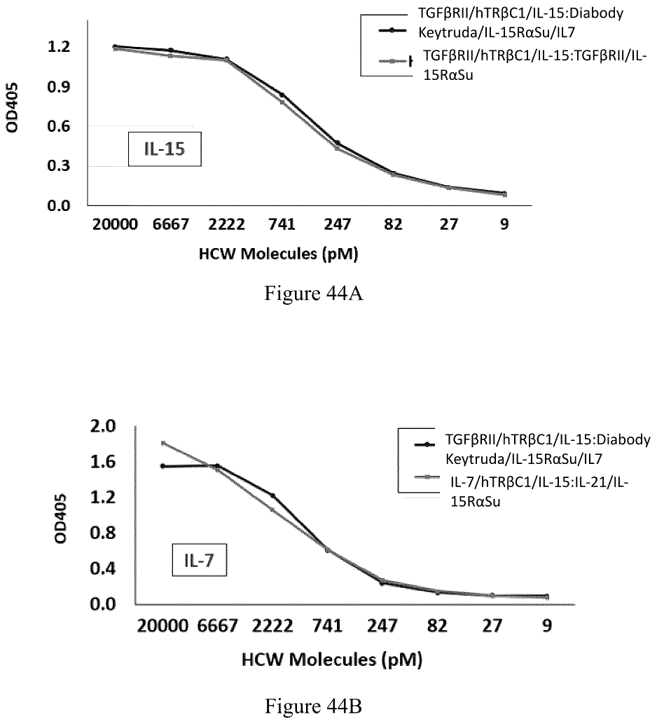

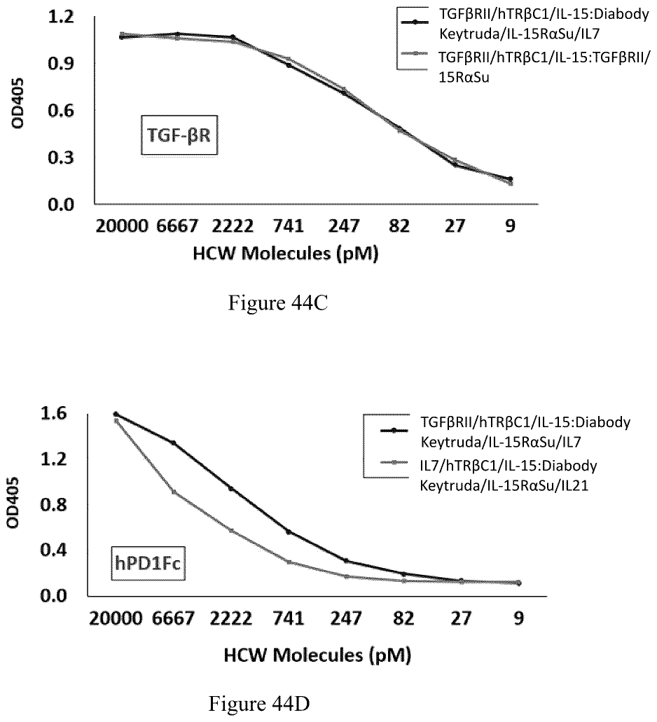

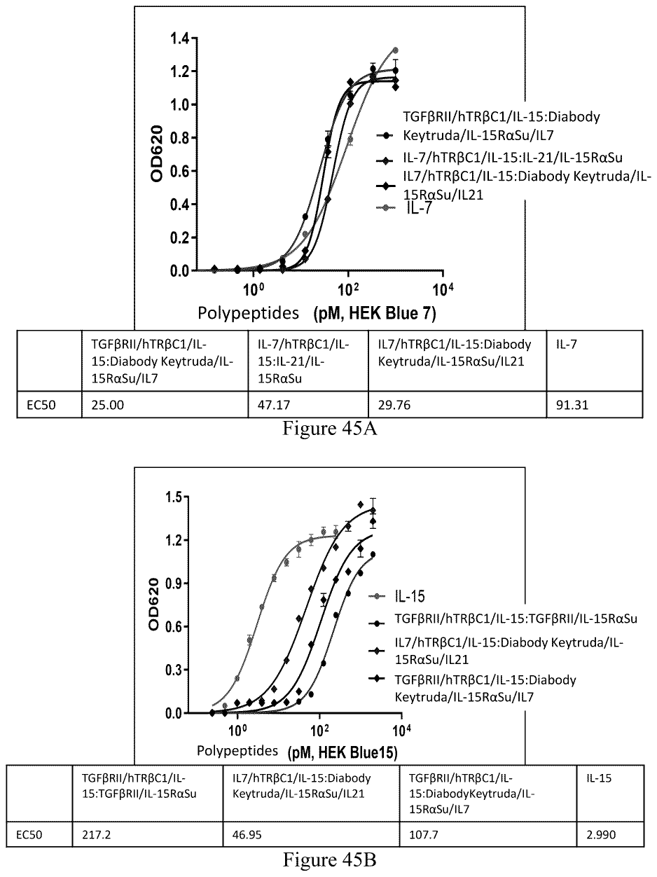

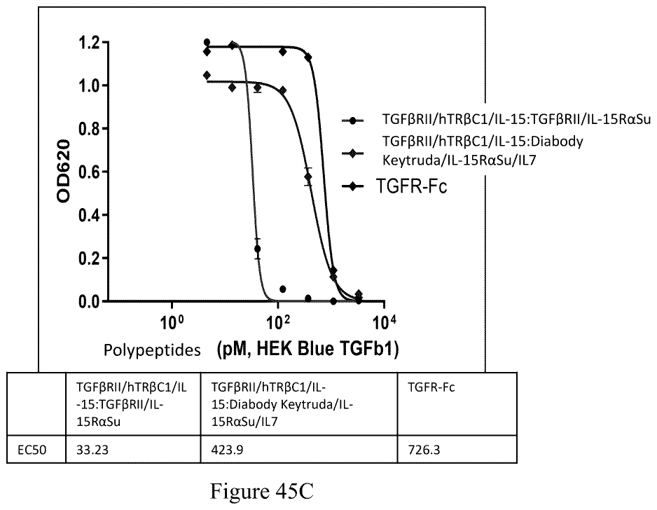

TGFβRII/hTRβC1/IL-15:Diabody Keytruda/IL-15RαSu/IL-7 (also referred to as TGFβRII/hTRβC1/IL-15:humanized anti-human PD-1 diabody/IL-15RαSu/IL-7) is illustrated in FIG. 40 and exhibits molecular weight 347.5 kDa in size exclusion chromatography (SEC, FIG. 43). The IL-7, IL-15 and TGFβR domains of TGFβRII/hTRβC1/IL-15:Diabody Keytruda/IL-15RαSu/IL-7 were confirmed with ELISA based on binding activities to correspondent antibodies (FIGS. 44A-C). The PD-1 binding of diabody Keytruda domain was demonstrated with ELISA based on biotinylated PD-1-Fc fusion protein (FIG. 44D). The biological activities of IL-7, IL-15 and TGFb1 trap of TGFβRII/hTRβC1/IL-15:Diabody Keytruda/IL-15RαSu/IL-7 were evaluated with their reporter cell lines and showed that EC50 was 25 pM for IL-7, 108 pM for IL-15 and 424 pM for TGFb trap (FIGS. 45A-C). Thus, all of the fusion domains in the TGFβRII/hTRβC1/IL-15:Diabody αPD-1/IL-15RαSu/IL-7 complex are present and functional.

The nucleic acid and protein sequences of a TGFβRII/hTRβC1/IL-15:Diabody Keytruda/IL-15RαSu/IL-7 complex are shown below.

The nucleic acid sequence of the TGFβRII/hTRβC1/IL-15 construct (including the leader sequence) is as follows (SEQ ID NO: 195):

(Leader)
ATGGGAGTGAAAGTTCTTTTTGCCCTTATTTGTATTGCTGTGGCCGAGGCC
(TGFβRII-1)
ATCCCACCGCACGTTCAGAAGTCGGTTAATAACGACATGATAGTCACTGACAACAACGGTGCAG
TCAAGTTTCCACAACTGTGTAAATTTTGTGATGTGAGATTTTCCACCTGTGACAACCAGAAATC
CTGCATGAGCAACTGCAGCATCACCTCCATCTGTGAGAAGCCACAGGAAGTCTGTGTGGCTGTA
TGGAGAAAGAATGACGAGAACATAACACTAGAGACAGTTTGCCATGACCCCAAGCTCCCCTACC
ATGACTTTATTCTGGAAGATGCTGCTTCTCCAAAGTGCATTATGAAGGAAAAAAAAAAGCCTGG
TGAGACTTTCTTCATGTGTTCCTGTAGCTCTGATGAGTGCAATGACAACATCATCTTCTCAGAA
GAATATAACACCAGCAATCCTGAC
(Linker)
GGCGGAGGTGGAAGT
(TGFβRII-2)
ATCCCACCGCACGTTCAGAAGTCGGTTAATAACGACATGATAGTCACTGACAACAACGGTGCAG
TCAAGTTTCCACAACTGTGTAAATTTTGTGATGTGAGATTTTCCACCTGTGACAACCAGAAATC
CTGCATGAGCAACTGCAGCATCACCTCCATCTGTGAGAAGCCACAGGAAGTCTGTGTGGCTGTA
TGGAGAAAGAATGACGAGAACATAACACTAGAGACAGTTTGCCATGACCCCAAGCTCCCCTACC
ATGACTTTATTCTGGAAGATGCTGCTTCTCCAAAGTGCATTATGAAGGAAAAAAAAAAGCCTGG
TGAGACTTTCTTCATGTGTTCCTGTAGCTCTGATGAGTGCAATGACAACATCATCTTCTCAGAA
GAATATAACACCAGCAATCCTGAC
(TRBC1)
GAGGACCTGAACAAGGTGTTCCCACCCGAGGTCGCTGTGTTTGAGCCATCAGAAGCAGAGATCT
CCCACACCCAAAAGGCCACACTGGTGTGCCTGGCCACAGGCTTCTTCCCCGACCACGTGGAGCT
GAGCTGGTGGGTGAATGGGAAGGAGGTGCACAGTGGGGTCAGCACGGACCCGCAGCCCCTCAAG
GAGCAGCCCGCCCTCAATGACTCCAGATACTGCCTGAGCAGCCGCCTGAGGGTCTCGGCCACCT
TCTGGCAGAACCCCCGCAACCACTTCCGCTGTCAAGTCCAGTTCTACGGGCTCTCGGAGAATGA
CGAGTGGACCCAGGATAGGGCCAAACCCGTCACCCAGATCGTCAGCGCCGAGGCCTGGGGTAGA
GCAGAC
(IL-15)
AACTGGGTGAATGTAATAAGTGATTTGAAAAAAATTGAAGATCTTATTCAATCTATGCATATTG
ATGCTACTTTATATACGGAAAGTGATGTTCACCCCAGTTGCAAAGTAACAGCAATGAAGTGCTT
TCTCTTGGAGTTACAAGTTATTTCACTTGAGTCCGGAGATGCAAGTATTCATGATACAGTAGAA
AATCTGATCATCCTAGCAAACAACAGTTTGTCTTCTAATGGGAATGTAACAGAATCTGGATGCA
AAGAATGTGAGGAACTGGAGGAAAAAAATATTAAAGAATTTTTGCAGAGTTTTGTACATATTGT
CCAAATGTTCATCAACACTTCTTGATAACGCGTACGAAG.

The nucleic acid sequence of the Diabody Keytruda/IL-15RαSu/IL-7 construct (including the leader sequence) is as follows (SEQ ID NO: 196):

(Leader)
ATGGGAGTGAAAGTTCTTTTTGCCCTTATTTGTATTGCTGTGGCCGAGGCC
(DbPD-1)
CAAGTTCAACTCGTGCAGAGCGGTGTGGAAGTTAAAAAGCCCGGCGCTAGCGTAAAGGTGAGCT
GTAAGGCTAGCGGCTACACCTTCACCAATTACTACATGTACTGGGTTAGGCAAGCCCCTGGACA
AGGCTTAGAATGGATGGGTGGGATAAACCCTTCGAATGGTGGAACCAACTTTAACGAGAAGTTC
AAAAACAGAGTGACGTTGACCACCGACTCTAGCACCACTACCGCGTACATGGAACTGAAGAGCC
TGCAGTTCGATGATACAGCGGTGTATTATTGCGCCCGAAGAGACTACCGATTCGACATGGGGTT
TGACTACTGGGGCCAAGGCACCACCGTCACCGTCAGCAGCGGCGGAGGCGGTAGTGAGATTGTT
CTGACCCAAAGCCCTGCCACCCTCTCGTTAAGCCCGGGGGAGAGAGCAACCCTGAGCTGTAGAG
CTAGCAAGGGCGTGAGCACCTCGGGCTACAGTTACCTGCACTGGTACCAACAGAAGCCTGGCCA
AGCCCCTCGATTATTGATCTACTTAGCTAGCTACTTAGAAAGCGGCGTGCCCGCTAGATTCAGC
GGCAGCGGGAGCGGCACCGACTTTACACTGACGATAAGTAGCTTAGAGCCTGAGGACTTTGCAG
TGTATTACTGCCAACATTCCCGGGACCTGCCGCTTACCTTCGGGGGCGGCACCAAAGTGGAGAT
TAAAGGAGGCGGAGGGTCGGGCGGTGGTGGGTCTGGAGGTGGAGGAAGCCAAGTGCAGCTGGTG
CAGAGCGGCGTGGAGGTGAAGAAGCCGGGCGCTAGCGTGAAGGTGAGCTGCAAGGCCTCCGGAT
ATACCTTCACAAACTACTACATGTATTGGGTTAGACAAGCCCCTGGTCAAGGCCTGGAGTGGAT
GGGCGGTATAAACCCTTCCAATGGAGGTACTAATTTCAACGAAAAATTCAAGAACCGTGTGACC
CTGACCACCGACAGTAGCACCACCACTGCATACATGGAGCTGAAGAGTCTGCAGTTCGACGATA
CCGCGGTCTACTACTGTGCCCGACGAGACTATAGATTTGACATGGGCTTCGACTACTGGGGACA
AGGCACTACGGTGACCGTGAGCAGCGGTGGCGGCGGCTCCGAAATTGTGCTGACACAGTCGCCT
GCAACCCTGTCCTTGAGCCCTGGCGAGCGGGCAACTCTGAGCTGCCGAGCTAGCAAAGGCGTGA
GTACAAGCGGCTATAGCTACCTACATTGGTATCAGCAAAAGCCTGGCCAAGCCCCGCGACTGCT
GATCTACCTGGCTAGCTACCTGGAGAGCGGCGTTCCTGCTAGATTCAGCGGATCTGGCTCTGGT
ACTGACTTCACCTTAACTATCAGTAGCCTTGAACCCGAGGACTTCGCTGTGTACTATTGTCAGC
ACAGCAGAGACCTCCCTCTGACCTTTGGAGGTGGTACCAAGGTCGAAATCAAA
(IL-15αSushi)
ATCACGTGCCCTCCCCCCATGTCCGTGGAACACGCAGACATCTGGGTCAAGAGCTACAGCTTGT
ACTCCAGGGAGCGGTACATTTGTAACTCTGGTTTCAAGCGTAAAGCCGGCACGTCCAGCCTGAC
GGAGTGCGTGTTGAACAAGGCCACGAATGTCGCCCACTGGACAACCCCCAGTCTCAAATGCATT
AGA
(IL-7)
GATTGTGATATTGAAGGTAAAGATGGCAAACAATATGAGAGTGTTCTAATGGTCAGCATCGATC
AATTATTGGACAGCATGAAAGAAATTGGTAGCAATTGCCTGAATAATGAATTTAACTTTTTTAA
AAGACATATCTGTGATGCTAATAAGGAAGGTATGTTTTTATTCCGTGCTGCTCGCAAGTTGAGG
CAATTTCTTAAAATGAATAGCACTGGTGATTTTGATCTCCACTTATTAAAAGTTTCAGAAGGCA
CAACAATACTGTTGAACTGCACTGGCCAGGTTAAAGGAAGAAAACCAGCTGCCCTGGGTGAAGC
CCAACCAACAAAGAGTTTGGAAGAAAATAAATCTTTAAAGGAACAGAAAAAACTGAATGACTTG
TGTTTCCTAAAGAGACTATTACAAGAGATAAAAACTTGTTGGAATAAAATTTTGATGGGCACTA
AAGAACACTGA.

### Example 21: Binding Activity of Keytruda Diabody of TRBC Molecules versus Keytruda to hPD-1 Using Jurkat-Raji PD-1/PD-L1 assay

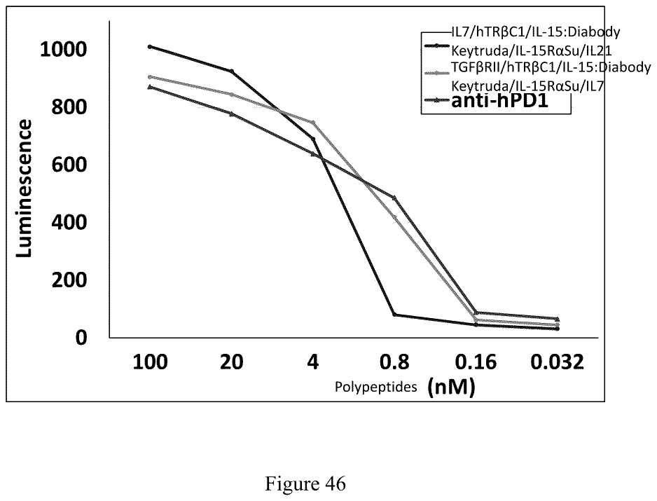

Anti-hPD-1 reagents, Jurkat-Lucia-PD-1 cells, and Raji-hPD-L1 cells were added to wells of 96-well plate. The plate was incubated at 37° C. for 6 hours. 20 μL of the supernatant was transferred into a 96-well black plate. 50 μL of luminescence assay reagent was added to detect activation of the Jurkat-Lucia-PD-1 cells. Luminescent activity was read using a luminometer. The blocking activity (FIG. 46) of PD-1-PDL1 by TGFβRII/hTRβC1/IL-15:Diabody Keytruda/IL-15RαSu/IL-7 was comparable with commercial Keytruda (SelleckChem).

### Example 22: Demonstration of Checkpoint Blockade Activity of IL-7/hTRpC1/IL-15:Diabody Keytruda/IL-15RαSu/IL-21

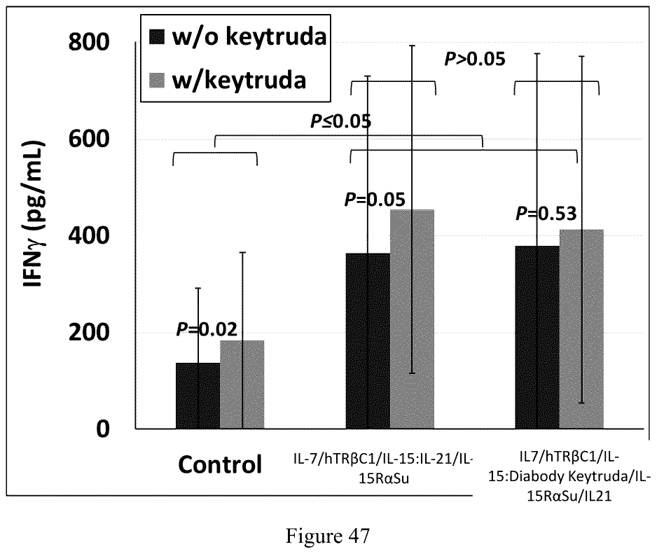

Human PBMCs (n=6) were cultured for 4 days in the presence of IL-15 and anti-CD3. The activated cells were re-cultured in the presence of Raji-PDL1 and multi-chain chimeric polypeptides. IFNγ produced by activated PBMCs in the supernatants were determined with FCM (Cytometric Bead Assay, CBA). IL-7/hTRβC1/IL-15:IL-15RαSu/IL-21 and IL-7/hTRβC1/IL-15:Diabody Keytruda/IL-15RαSu/IL-21 similarly stimulated human PBMCs to produce IFNγ (p≤0.05). Keytruda alone stimulated human PBMCs to produce IFNγ (p=0.02) and enhanced the ability of IL-7/hTRβC1/IL-15:IL-15RαSu/IL-21 to stimulate PBMCs to produce IFNγ (p=0.05). However, Keytruda did not further enhance IFNγ production mediated by IL-7/hTRβC1/IL-15:Diabody Keytruda/IL-15RαSu/IL-21 because IL-7/hTRβC1/IL-15:Diabody Keytruda/IL-15RαSu/IL-21 contains Keytruda function. (FIG. 47.)

### Example 23: Cytotoxic Activity of Activated Human PBMCs by Multi-Chain Chimeric Polypeptides against Pancreatic Tumor Cells

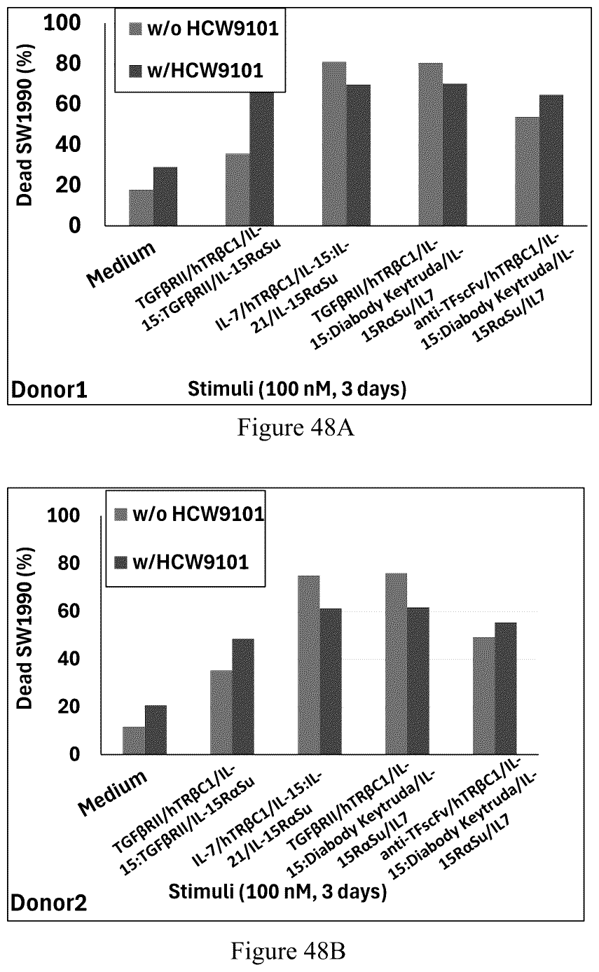

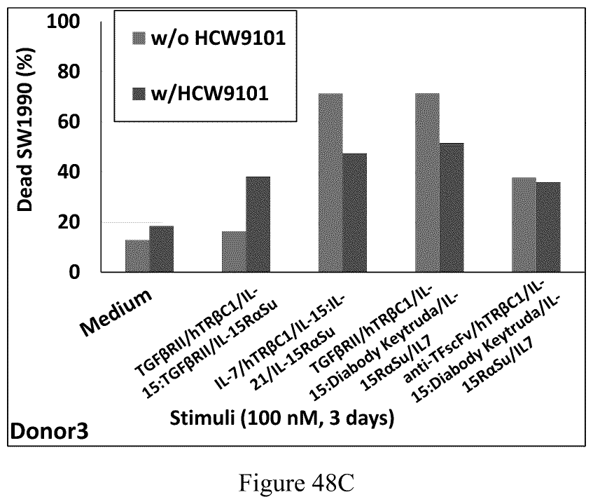

Fresh human PBMCs were isolated from human blood. Human PBMCs were incubated in R10 with multi-chain chimeric polypeptides at 100 nM for 3 days. Activated PBMCs and Cellltrace violet-labeled SW1990 cells were mixed and incubated for 20 hours. The activated cells were used as effector cells and SW1990 cells were used as target cells. The cytotoxic activity (%) of PBMCs was evaluated with FCM after 20 hrs of mixed culture of effector and target. The cultures were harvested and resuspended in HBSS with PI-2 μg/mL. Dead SW1990 cells (Celltrace+PI+) were counted by FCM, and the percentage of dead cells was calculated. (FIGS. 48A-C.). The results show that multi-chain chimeric polypeptides were capable of inducing cytotoxic activity of human PBMCs against SW1990 human pancreatic tumor cells.

### Example 24: Multi-Chain Chimeric Polypeptides Induce Activation and Proliferation of Human PMBCs

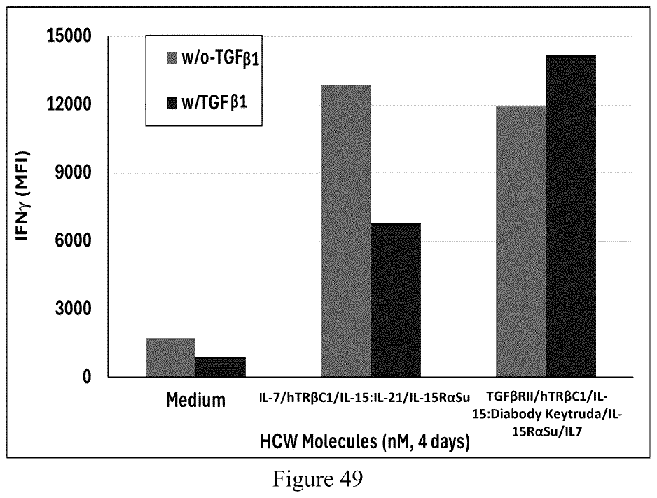

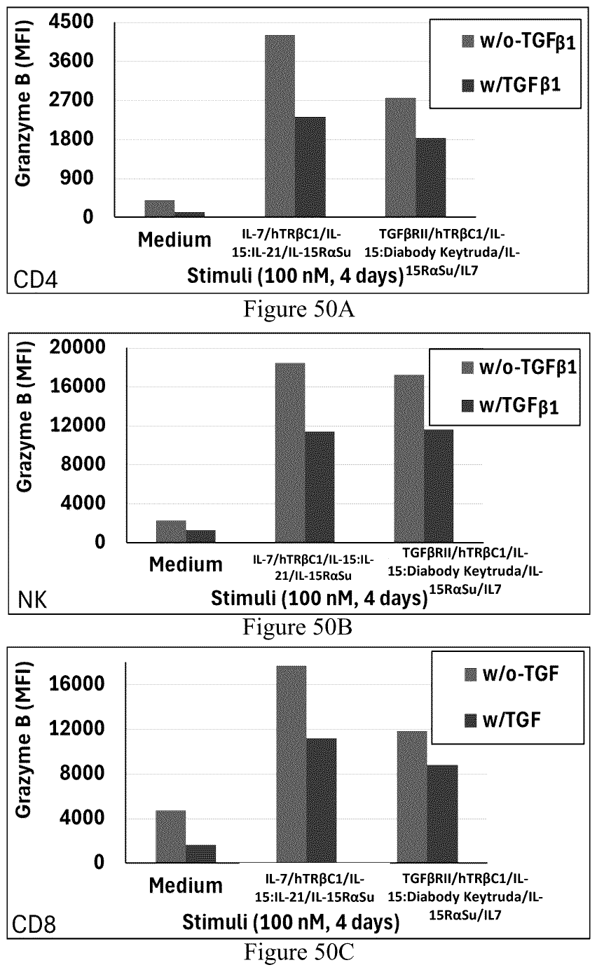

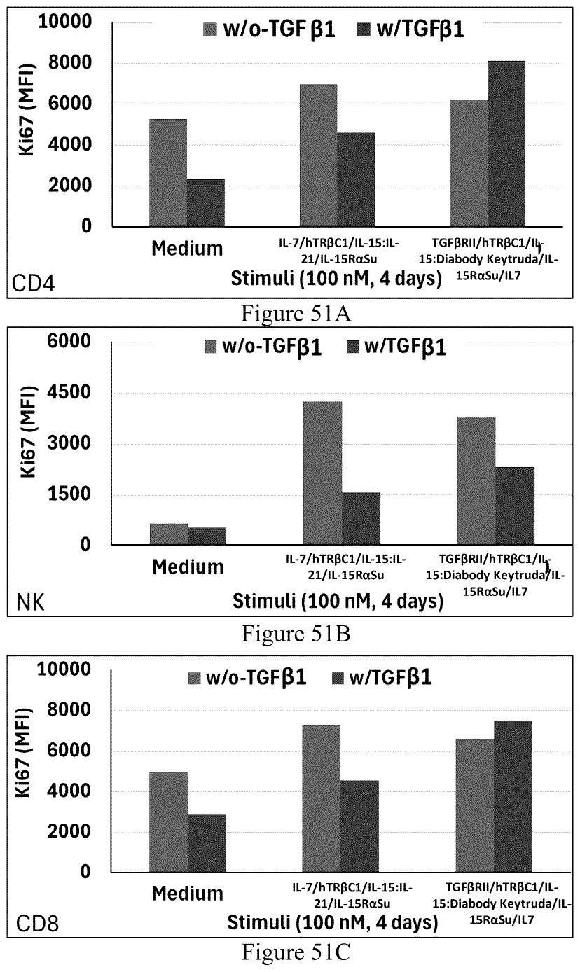

Frozen human PBMCs were incubated in R10 with multi-chain chimeric polypeptides at 100 nM for 4 days (hPBMCs TGFβ-4 nM, multi-chain chimeric polypeptides-100 nM): 4×10⁶ cells/2 mL/well in 12-well plates for 4 days). The PBMCs were harvested and IFNγ production (activation), granzyme B expression (activation), and Ki67 expression (proliferation) were evaluated in PBMC subsets by FCM. See FIGS. 49, 50A-C, 51A-C. The results show that multi-chain chimeric polypeptides were capable of inducing activation and proliferation of human CD4⁺ and CD8⁺ T cells and NK cells. Additionally, TGFβRII in multi-chain chimeric polypeptides reduced inhibition of TGFβ1 in the cultures.

### Example 25. Analysis of Tissue Factor Expression in Human Cancer Cell Lines

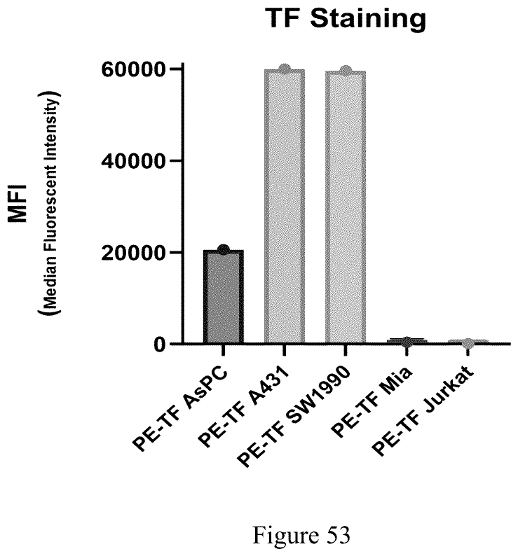

100,000 cells of each human cancer cell line (Jurkat, A431, AsPC1, MiaPaCa2, SW1990) were incubated with 1 μg of biotinylated-HCW9101 for Tissue Factor (TF) staining for 30 minutes at room temperature. After washing with FACS buffer, cells were incubated with 1 μL of streptavidin-phycoerythrin (SA-PE) (for TF) for 30 minutes at 4° C. Following a final wash with FACS buffer, samples were analyzed by flow cytometry. Mean Fluorescence Intensity (MFI) for PE was recorded for TF. Based on PE median fluorescence intensity, this method demonstrated the highest surface TF expression in A431 and SW1990 cells, moderate expression in AsPC-1 cells, and minimal to undetectable levels in MIA PaCa-2 cells. See FIG. 53.

### Example 26. Quantitative Flow Cytometric Analysis of Tissue Factor Expression Using PE Calibration Beads

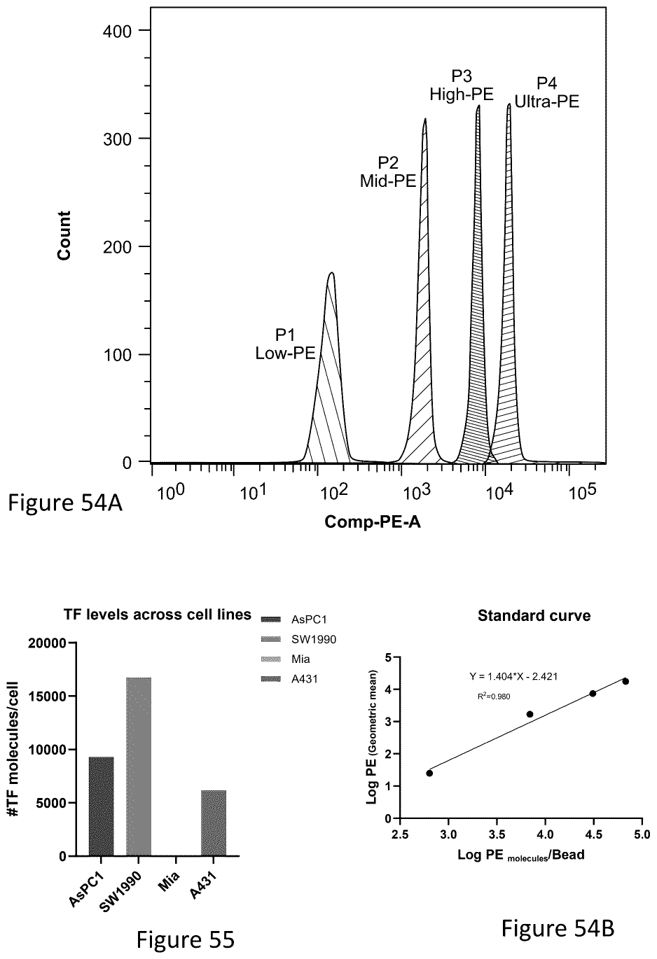

Quantibrite PE Standard Curve Preparation: Quantibrite PE beads were resuspended in 0.5 mL of FACS buffer containing 0.5% sodium azide. Flow cytometry was used to acquire a total of 10,000 events (FIG. 55A). The PE fluorescence intensities of the four bead populations were recorded, and the geometric mean fluorescence intensity was calculated for each population. A standard curve was then generated by plotting the log-transformed geometric mean PE intensity (Y-axis) against the log-transformed number of PE molecules per bead, as provided by the manufacturer (X-axis) (FIG. 54B).

Quantification of Surface Tissue Factor (TF) Expression in Pancreatic and Epidermoid Carcinoma CellLines: ASPC-1, SW1990, A431, and MIA PaCa-2 cells were stained with BN9101 (anti-TF), followed by detection with streptavidin-PE. Secondary-only controls were included. Cells were analyzed by flow cytometry using the same instrument settings as for the BD Quantibrite™ PE bead calibration. The geometric mean PE fluorescence intensities were log-transformed and interpolated against the Quantibrite standard curve to determine the number of PE molecules bound per cell, representing surface TF density. Results illustrate differential TF expression across the tested cell lines. A similar TF expression pattern was observed using Quantibrite-based quantification, confirming prior TF staining results based on MFI, which were more qualitative in nature. (FIG. 55).

### Example 27: T-Cell Engager TGFβRII/hTRβC1/IL-15:Anti-TFscFv/IL-15RαSu/Anti-CD3scFv-Mediated Conjugation of Jurkat T Cells with Multiple Tumor Cell Lines

For the T-cell engager-mediated conjugation experiment, Jurkat T cells were stained with PECy7-CD45, and A431 cancer cells were stained with CellTrace Violet (CTV) following the manufacturer recommended protocol. A total of 50,000 cells from both Jurkat and A431 were mixed in the presence of the T-cell engager TGFβRII/hTRβC1/IL-15:anti-TFscFv/IL-15RαSu/anti-CD3scFv (which targets anti-CD3 and anti-TF; see Example 31) at various concentrations. The cell mixture was incubated at 37° C. for 30 minutes in RPMI1640 supplemented with 10% FBS. After incubation, the cells were washed with FACS buffer and analyzed by flow cytometry. The PECy7-CD45 signal on the Y-axis identifies Jurkat-positive cells (Q1), while the CTV signal on the X-axis identifies A431 cancer cells (Q4).

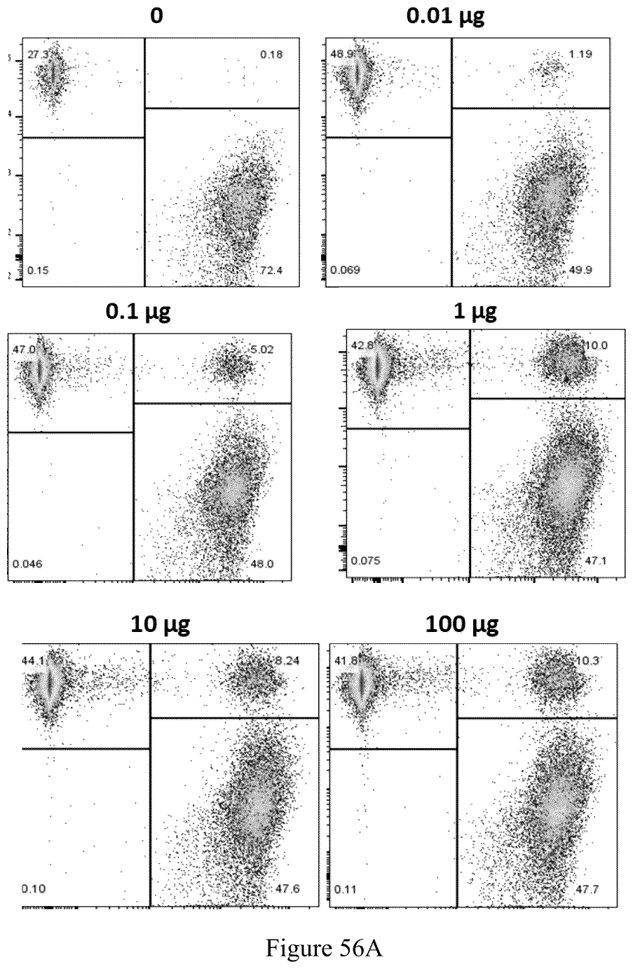

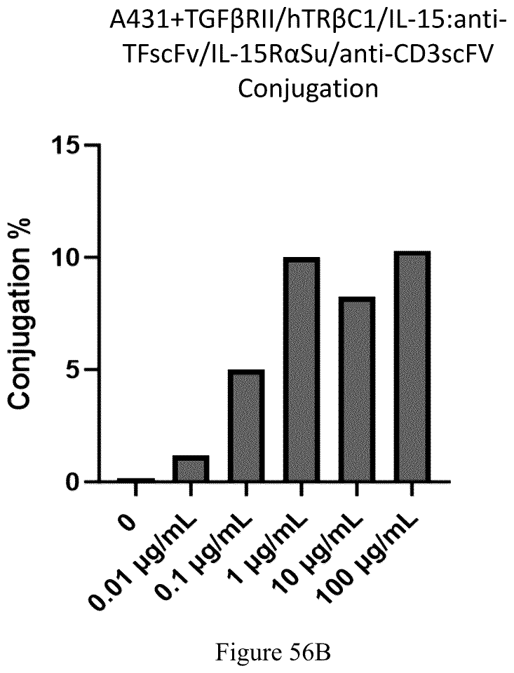

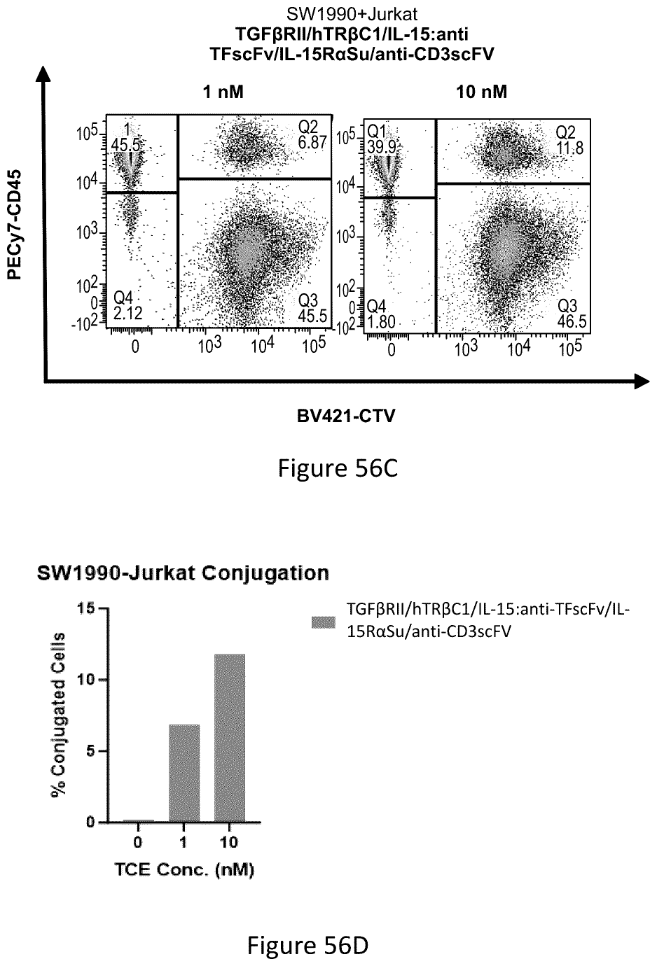

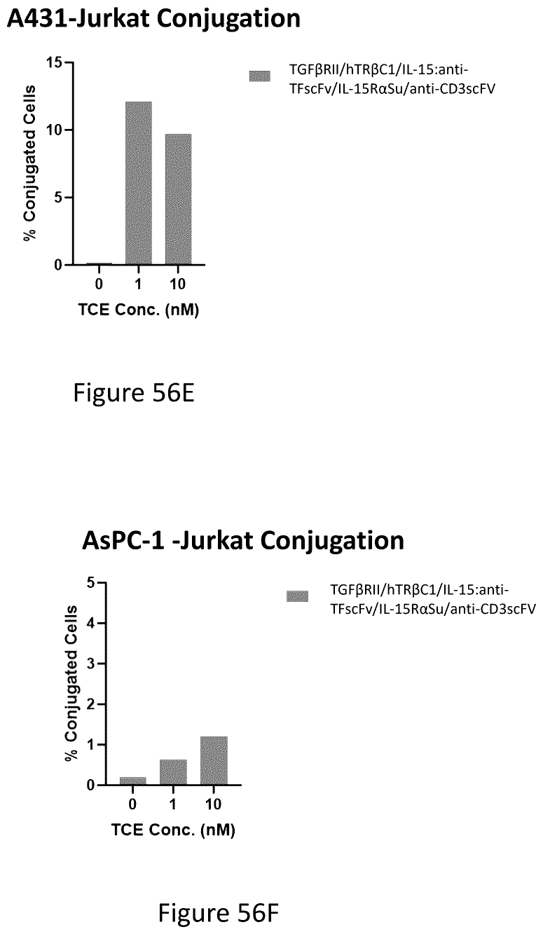

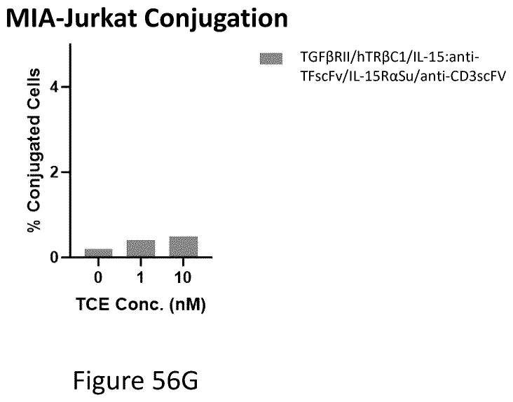

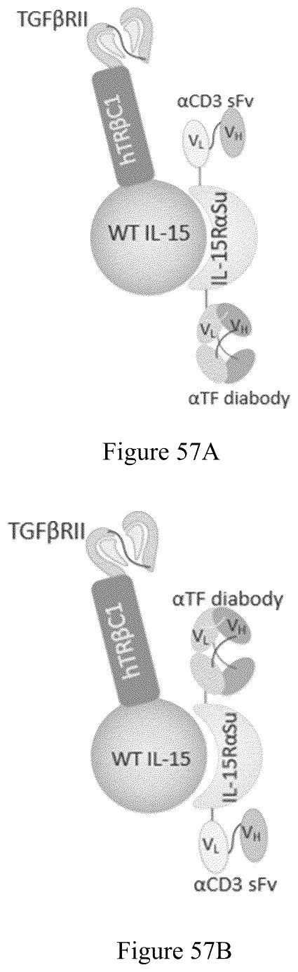

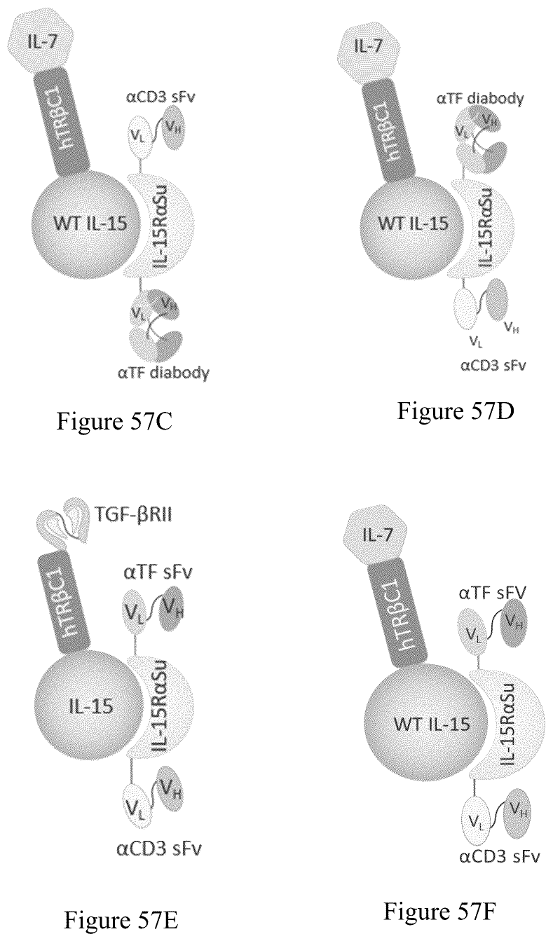

Double-positive cells (conjugated Jurkat and cancer cells) were seen in Q2, indicating successful conjugation mediated by the T-cell engager. See FIGS. 56A-B.

To further optimize and evaluate target specificity, the assay was repeated using two concentrations of TGFβRII/hTRβC1/IL-15:anti-TF scFv/IL-15RαSu/anti-CD3 scFv (1 nM and 10 nM) with A431 and additional tumor cell lines including SW1990, AsPC-1, and MIA PaCa-2. Equal numbers of Jurkat and tumor cells were co-incubated for 30-60 minutes at 37° C. and analyzed similarly. Quantitative analysis revealed high conjugate formation in A431 and SW1990 cells, moderate levels in AsPC-1, and no detectable conjugation in MIA PaCa-2 cells, consistent with their differential expression of the target antigen (FIGS. 56C-56G). These results confirm the target-dependent activity of TGFβRII/hTRβC1/IL-15:anti-TF scFv/IL-15RαSu/anti-CD3 scFv across multiple tumor models while validating reproducibility in A431 cells.

### Example 28: Activated T Cells to Release IL-2 by T Cell Engagers (TCEs)

AsPC-1 (TF+) or MIA-Pa-Ca-2 (TF−) cells (1.2×10⁴ cells/well) or culture medium (200 μl 110) were added in flat-bottom 96-well plate (Corning). After 24 h of adherent culture, the medium was removed. 100 μl of effector cells (PBMC) at E:T ratio of 10:1, 1.2×10⁵ cells/well in R10 from 3 donors and 100 μl TGFβRII/hTRβC1/IL-15:anti-CD3scFv/IL-15RαSu/anti-TF(diabody), TGFβRII/hTRβC1/IL-15:anti-TF(diabody)/IL-15RαSu/anti-CD3scFv, IL-7/hTRβC1/IL-15:anti-CD3scFv/IL-15RαSu/anti-TF(diabody), IL-7/hTRβC1/IL-15:anti-TF(diabody)/IL-15RαSu/anti-CD3scFv, or HCW9101 (final concentration 100 nM for each multi-chain chimeric polypeptide) were added into the well and co-incubated for 20 h. Supernatants were collected. IL-2 released into the supernatant were measured using IL-2 immunoassay kit (BioLegend).

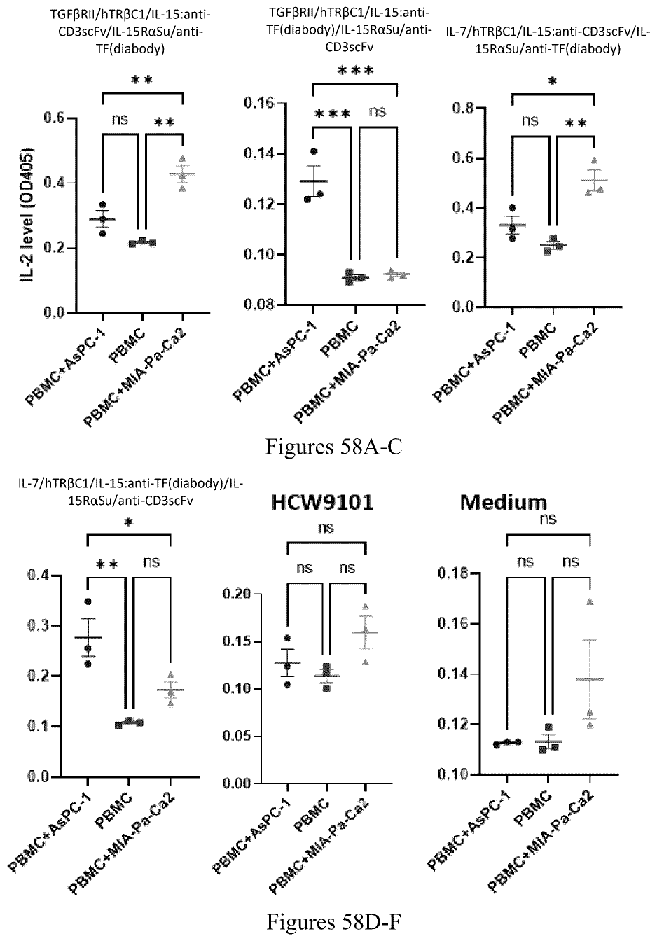

FIGS. 58A-F showed TGFβRII/hTRβC1/IL-15:anti-TF(diabody)/IL-15RαSu/anti-CD3scFv and IL-7/hTRβC1/IL-15:anti-TF(diabody)/IL-15RαSu/anti-CD3scFv had more specific activation of T cells to release IL-2 compared to TGFβRII/hTRβC1/IL-15:anti-CD3scFv/IL-15RαSu/anti-TF(diabody) and IL-7/hTRβC1/IL-15:anti-CD3scFv/IL-15RαSu/anti-TF(diabody). The results suggested that TCE complex with anti-TF antibody at the N-terminal position and anti-CD3 antibody at the C-terminal position would be an optimal format for the TRBC molecule design.

### Example 29: Evaluation of Cytotoxic Effects on AsPC-1 and MIA Cell Lines by Human PBMCs Activated via T-Cell Engagers

CellTrace Violet kit (Invitrogen) labeled AsPC-1 (TF+) or MIA-Pa-Ca-2 (TF−) cells (3×10⁴ cells/well) were mixed with PBMCs from 4 donors (3×10⁵ cells/well) in the presence of TGFβRII/hTRβC1/IL-15:anti-TF scFv/IL-15RαSu/anti-CD3 scFv, TGFβRII/hTRβC1/IL-15:anti-TF(diabody)/IL-15RαSu/anti-CD3scFv, IL-7/hTRβC1/IL-15:anti-CD3scFv/IL-15RαSu/anti-TF(diabody), IL-7/HTRBC1/IL-15:IL-21/IL-15RαSu, TGFBRII/HTRBC1/IL-15:TGFBRII/IL-15RαSu, or HCW9101 (100 nM each), in a V-shape 96-well plate (Corning). After 20 h incubation, cell mixes were spun down and resuspended in complete-medium with propidium iodide (PI, ex/em=488/610) at 2 μg/mL and analyzed with FACSCelestra. The percentage of target cell killing were evaluated through the appearance of PI+ and CellTrace Violet+dead cells.

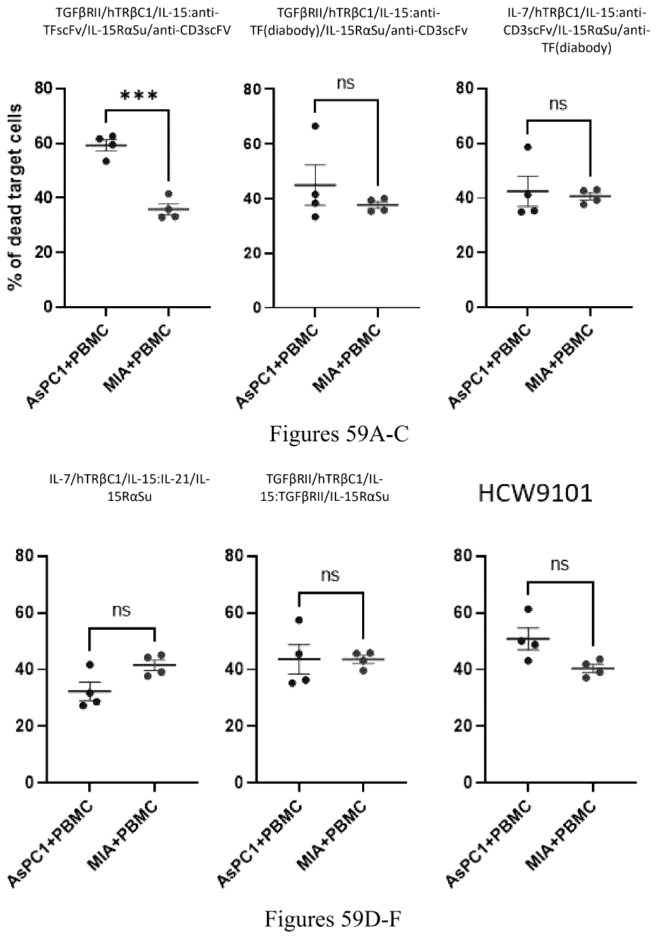

See FIGS. 59A-F. The data demonstrated that only TGF PRII/hTRβC1/IL-15:anti-TF scFv/IL-15RαSu/anti-CD3 scFv engaged specific killing of TF+ target cell by human PBMCs.

### Example 30: IL-7/hTRpC1/IL-15 and αTFAb/IL-15RαSu/αCD3Ab Fusion Proteins

A protein complex was generated comprising anti-TFscFv/IL-15RαSu/anti-CD3scFv and IL-7/hTRβC1/IL-15 fusion proteins. The mouse immunoglobulin H chain leader sequences were obtained from GenBank: AH002574.2. The N-terminal native G. princeps signal peptide sequences were obtained from O'Neill et al, ACS Synth Biol. 2023, 2(8):2339. The linker sequences were obtained from Huston et al, PNAS 1988, 85(16):5879. The anti-CD3 sequences were obtained from GENBANK: CAH69219.1. The humanized anti-TF sequences were obtained from U.S. Pat. No. 7,968,094. The human TRBC1 sequences were obtained from GENBANK: M12887.1. The human IL-15RαSu sequences were obtained from NCBI REFERENCE SEQUENCE: NM_002189.4. The human IL-15 sequences were obtained from NCBI Reference Sequence: NM_172175.3 and human IL-7 sequences were obtained from GENBANK: J04156.1. DNA for these sequences was synthesized by Genewiz. Specifically, constructs were made linking anti-TF scFv to the N-terminus of IL-15RαSu and linking anti-CD3scFv to the C-terminus of IL-15RαSu. The nucleic acid and protein sequences of a construct comprising anti-TF scFv linked to the N-terminus of IL-15RαSu and anti-CD3scFv linked to the C-terminus of IL-15RαSu are shown below.

The nucleic acid sequence of the anti-TFscFv/IL-15RαSu/anti-CD3scFv construct (including the mouse immunoglobulin H chain leader sequence) is as follows (SEQ ID NO: 150):

(Mouse immunoglobulin H chain signal peptide)
ATGGGCTGGAGCTGCATCATCCTGTTCCTGGTGGCCACCGCCACCGGCGTGCACTCC
(Humanized anti-human TFscFv)
GACATACAGATGACCCAAAGCCCTGCTAGCCTGAGCGCGAGCGTGGGCGACAGAGTGACCATCA
CCTGCCTCGCCTCTCAGACGATTGACACCTGGCTGGCTTGGTATTTGCAGAAACCTGGGAAAAG
CCCTCAGCTGCTGATCTACGCAGCCACCAATTTAGCCGATGGCGTCCCTTCTAGATTTAGCGGC
TCGGGATCCGGCACCGATTTTAGCTTTACAATTTCATCTCTACAGCCTGAGGACTTCGCCACCT
ATTATTGTCAACAAGTATATAGCAGCCCTTTCACCTTCGGCCAAGGCACCAAGTTAGAGATCAA
GGGCGGTGGCGGCTCCGGGGGCGGGGGAAGTGGAGGTGGGGGGTCACAGATTCAGCTGGTTCAA
AGCGGCGGCGAGGTGAAGAAACCTGGCGCCTCGGTGAGAGTTAGCTGCAAAGCTAGCGGATATT
CGTTTACTGACTACAACGTGTATTGGGTGAGGCAGAGCCCGGGCAAGGGACTGGAGTGGATTGG
GTACATTGACCCCTACAACGGCATAACCATATATGATCAGAACTTCAAGGGCAAAGCCACGCTG
ACCGTGGATAAAAGCACAAGCACCGCTTATATGGAGTTGAGCTCCCTCCGTAGCGAAGACACGG
CAGTATACTTTTGCGCACGGGATGTGACCACGGCCCTAGATTTCTGGGGACAAGGGACAACCGT
TACTGTTAGTTCC
(Linker)
GGCGGTGGGGGCTCG
(Human IL-15Rα sushi domain)
ATCACGTGCCCTCCCCCCATGTCCGTGGAACACGCAGACATCTGGGTCAAGAGCTACAGCTTGT
ACTCCAGGGAGCGGTACATTTGTAACTCTGGTTTCAAGCGTAAAGCCGGCACGTCCAGCCTGAC
GGAGTGCGTGTTGAACAAGGCCACGAATGTCGCCCACTGGACAACCCCCAGTCTCAAATGCATT
AGAGACCCTGCCCTGGTTCACCAAAGG
(Anti-human CD3scFv)
GATATTCAGATGACGCAGTCGCCAAGCTCACTCTCGGCCTCCGTAGGAGACAGAGTTACCATCA
CCTGCAGAGCTAGCCAAGACATCAGAAACTACCTGAACTGGTATCAGCAGAAGCCTGGCAAGGC
CCCTAAGCTCCTGATCTACTACACGAGCAGACTTGAGAGCGGGGTACCATCCCGCTTCAGCGGA
TCTGGAAGCGGCACAGACTACACCCTGACCATCTCTAGTCTGCAGCCTGAGGACTTCGCCACTT
ACTATTGTCAGCAAGGCAACACCCTGCCTTGGACCTTTGGCCAAGGCACCAAAGTTGAGATAAA
GGGAGGGGGAGGGAGCGGTGGCGGAGGCTCGGGTGGGGGTGGCTCAGAAGTACAGCTGGTTGAG
TCCGGCGGTGGTCTGGTGCAGCCTGGCGGAAGCCTGAGACTGAGCTGCGCCGCAAGCGGCTATA
GCTTCACTGGCTATACCATGAACTGGGTGCGCCAAGCCCCTGGCAAGGGCCTGGAATGGGTGGC
CCTGATCAACCCTTACAAGGGCGTGAGCACCTACAATCAGAAGTTCAAGGACAGATTCACCATC
AGCGTGGACAAGAGCAAGAACACCGCCTACCTGCAGATGAACAGCCTGAGAGCCGAGGACACTG
CGGTCTATTACTGTGCTAGAAGCGGCTACTACGGCGACAGCGACTGGTACTTCGACGTGTGGGG
GCAAGGCACCTTGGTAACGGTAAGCTCC

The amino acid sequence of the anti-TFscFv/IL-15RαSu/anti-CD3scFv fusion protein (including the mouse immunoglobulin H chain leader sequence) is as follows (SEQ ID NO: 175):

(Signal peptide)
MGWSCIILFLVATATGVHS
(Humanized anti-human TFscFv)
DIQMTQSPASLSASVGDRVTITCLASQTIDTWLAWYLQKPGKSPQLLIYAATNLADGVPSRFSG
SGSGTDFSFTISSLQPEDFATYYCQQVYSSPFTFGQGTKLEIKGGGGSGGGGSGGGGSQIQLVQ
SGGEVKKPGASVRVSCKASGYSFTDYNVYWVRQSPGKGLEWIGYIDPYNGITIYDQNFKGKATL
TVDKSTSTAYMELSSLRSEDTAVYFCARDVTTALDFWGQGTTVTVSS
(Linker)
GGGGS
(Human IL-15Rα sushi domain)
ITCPPPMSVEHADIWVKSYSLYSRERYICNSGFKRKAGTSSLTECVLNKATNVAHWTTPSLKCI
RDPALVHQR
(Anti-human CD3scFv)
DIQMTQSPSSLSASVGDRVTITCRASQDIRNYLNWYQQKPGKAPKLLIYYTSRLESGVPSRFSG
SGSGTDYTLTISSLQPEDFATYYCQQGNTLPWTFGQGTKVEIKGGGGSGGGGSGGGGSEVQLVE
SGGGLVQPGGSLRLSCAASGYSFTGYTMNWVRQAPGKGLEWVALINPYKGVSTYNQKFKDRFTI
SVDKSKNTAYLQMNSLRAEDTAVYYCARSGYYGDSDWYFDVWGQGTLVTVSS

Constructs were also made inking the human 1L-7 sequence to the -terminus of human TRBC1 following with the N-terminus coding region of IL-15 synthesized by Genewiz. The nucleic acid and protein sequences of a construct comprising human IL-7 linked to the N-terminus of human TRBC1 following with the N-terminus coding region of IL-15 are shown below.

The nucleic acid sequence of the IL-7/hTRβC1/IL-15 construct (including the N-terminal native G. princeps signal peptide sequence) is as follows (SEQ ID NO: 192):

(N-terminal native G. princeps signal peptide)
ATGGGCGTGAAGGTGCTGTTCGCCCTGATCTGCATCGCCGTGGCCGAGGCC
(Human IL-7)
GATTGTGATATTGAAGGTAAAGATGGCAAACAATATGAGAGTGTTCTAATGGTCAGCATCGATC
AATTATTGGACAGCATGAAAGAAATTGGTAGCAATTGCCTGAATAATGAATTTAACTTTTTTAA
AAGACATATCTGTGATGCTAATAAGGAAGGTATGTTTTTATTCCGTGCTGCTCGCAAGTTGAGG
CAATTTCTTAAAATGAATAGCACTGGTGATTTTGATCTCCACTTATTAAAAGTTTCAGAAGGCA
CAACAATACTGTTGAACTGCACTGGCCAGGTTAAAGGAAGAAAACCAGCTGCCCTGGGTGAAGC
CCAACCAACAAAGAGTTTGGAAGAAAATAAATCTTTAAAGGAACAGAAAAAACTGAATGACTTG
TGTTTCCTAAAGAGACTATTACAAGAGATAAAAACTTGTTGGAATAAAATTTTGATGGGCACTA
AAGAACAC
(Human TRBC1)
GAGGACCTGAACAAGGTGTTCCCACCCGAGGTCGCTGTGTTTGAGCCATCAGAAGCAGAGATCT
CCCACACCCAAAAGGCCACACTGGTGTGCCTGGCCACAGGCTTCTTCCCCGACCACGTGGAGCT
GAGCTGGTGGGTGAATGGGAAGGAGGTGCACAGTGGGGTCAGCACGGACCCGCAGCCCCTCAAG
GAGCAGCCCGCCCTCAATGACTCCAGATACTGCCTGAGCAGCCGCCTGAGGGTCTCGGCCACCT
TCTGGCAGAACCCCCGCAACCACTTCCGCTGTCAAGTCCAGTTCTACGGGCTCTCGGAGAATGA
CGAGTGGACCCAGGATAGGGCCAAACCCGTCACCCAGATCGTCAGCGCCGAGGCCTGGGGTAGA
GCAGAC
(Human IL-15)
AACTGGGTGAACGTGATCAGCGACCTGAAGAAGATCGAGGACCTGATTCAGAGCATGCACATCG
ACGCCACCCTGTACACCGAGAGCGACGTGCACCCTAGCTGCAAGGTGACCGCCATGAAGTGCTT
CCTGCTGGAGCTGCAAGTGATCAGCCTGGAGAGCGGCGACGCTAGCATCCACGACACCGTGGAG
AACCTGATCATCCTGGCCAACAACAGCCTGAGCAGCAACGGCAACGTGACCGAGAGCGGCTGCA
AGGAGTGCGAGGAGCTGGAGGAGAAGAACATCAAGGAGTTCCTGCAGAGCTTCGTGCACATCGT
GCAGATGTTCATCAACACAAGC

The amino acid sequence of the IL-7/hTRβC1/IL-15 fusion protein (including the N-terminal native G. princeps signal peptide sequence) is as follows (SEQ ID NO: 198):

(N-terminal native G. princeps signal peptide)
MGVKVLFALICIAVAEA
(Human IL-7)
DCDIEGKDGKQYESVLMVSIDQLLDSMKEIGSNCLNNEFNFFKRHICDANKEGMFLFRAARKLR
QFLKMNSTGDFDLHLLKVSEGTTILLNCTGQVKGRKPAALGEAQPTKSLEENKSLKEQKKLNDL
CFLKRLLQEIKTCWNKILMGTKEH
(Human TRBC1)
EDLNKVFPPEVAVFEPSEAFISHTQKATLVCLATGFFPDHVELSWWVNGKEVHSGVSTDPQPLK
EQPALNDSRYCLSSRLRVSATFWQNPRNHFRCQVQFYGLSENDEWTQDRAKPVTQIVSAEAWGR
AD
(Human IL-15)
NWVNVISDLKKIEDLIQSMHIDATLYTESDVHPSCKVTAMKCFLLELQVISLESGDASIHDTVE
NLIILANNSLSSNGNVTESGCKECEELEEKNIKEFLQSFVHIVQMFINTS

In some cases, the leader peptide is cleaved from the intact polypeptide to generate the mature form that may be soluble or secreted.

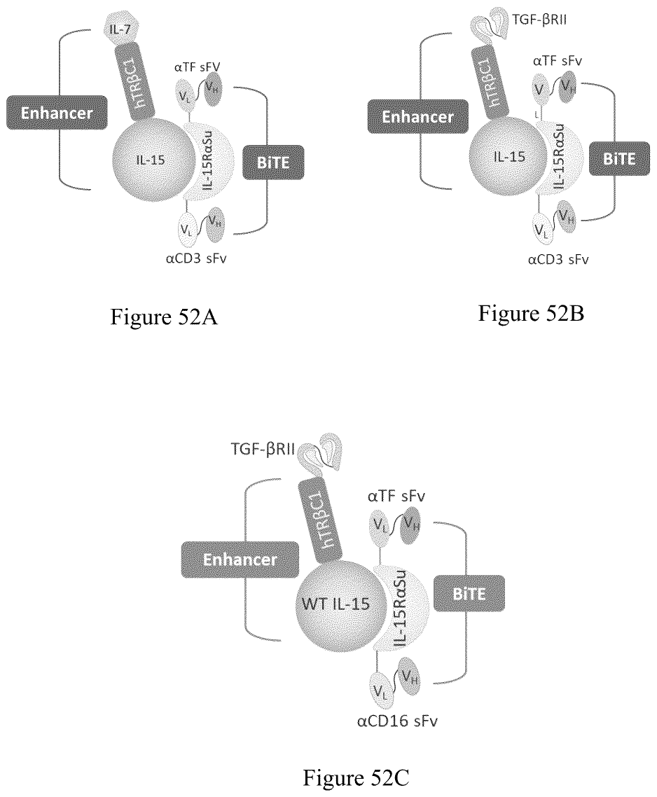

The anti-TFscFv/IL-15RαSu/anti-CD3scFv and IL-7/hTRβC1/IL-15 constructs were cloned into a modified retrovirus expression vectors pMSGV-1 as described previously (Hughes M S, Yu Y Y, Dudley M E, Zheng Z, Robbins P F, Li Y, et al. Transfer of a TCR gene derived from a patient with a marked antitumor response conveys highly active T-cell effector functions. Hum Gene Ther 2005; 16:457-72.), and the expression vectors were transfected into CHO-K1 cells. Co-expression of the two constructs in CHO-K1 cells allowed for formation and secretion of a soluble IL-7/hTRβC1/IL-15:anti-TFscFv/IL-15RαSu/anti-CD3scFv). The IL-7/hTRβC1/IL-15:anti-TFscFv/IL-15RαSu/anti-CD3scFv protein was purified from CHO cell culture supernatant by an anti-TRBC1 antibody (BF1) affinity chromatography and size exclusion chromatography resulting in soluble (non-aggregated) protein complexes consisting of anti-TFscFv/IL-15RαSu/anti-CD3scFv: IL-7/hTRβC1/IL-15 fusion proteins (FIG. 52A).

### Example 31: TGFR/hTRβC1/IL-15 and αTFAb/IL-15RαSu/αCD3Ab Fusion Proteins

A protein complex was generated comprising anti-TFscFv/IL-15RαSu/anti-CD3scFv and TGFR/hTRβC1/IL-15 fusion proteins. The mouse immunoglobulin H chain leader sequences were obtained from GenBank: AH002574.2. The N-terminal native G. princeps signal peptide sequences were obtained from O'Neill et al, ACS Synth Biol. 2023, 2(8):2339. The linker sequences were obtained from Huston et al, PNAS 1988, 85(16):5879. The anti-CD3 sequences were obtained from GENBANK: CAH69219.1. The humanized anti-TF sequences were obtained from U.S. Pat. No. 7,968,094. The human TRBC1 sequences were obtained from GENBANK: M12887.1. The human IL-15RαSu sequences were obtained from NCBI REFERENCE SEQUENCE: NM_002189.4. The human IL-15 sequences were obtained from NCBI Reference Sequence: NM_172175.3 and human TGF-beta type II receptor sequences were obtained from GENBANK: M85079.1. DNA for these sequences was synthesized by Genewiz. Specifically, constructs were made linking anti-TF scFv to the N-terminus of IL-15RαSu and linking anti-CD3scFv to the C-terminus of IL-15RαSu. The nucleic acid and protein sequences of a construct comprising anti-TF scFv linked to the N-terminus of IL-15RαSu and anti-CD3scFv linked to the C-terminus of IL-15RαSu are shown below.

The nucleic acid sequence of the anti-TFscFv/IL-15RαSu/anti-CD3scFv construct (including the mouse immunoglobulin H chain leader sequence) is as follows (SEQ ID NO: 150):

(Mouse immunoglobulin H chain signal peptide)
ATGGGCTGGAGCTGCATCATCCTGTTCCTGGTGGCCACCGCCACCGGCGTGCACTCC
(Humanized anti-human TFscFv)
GACATACAGATGACCCAAAGCCCTGCTAGCCTGAGCGCGAGCGTGGGCGACAGAGTGACCATCA
CCTGCCTCGCCTCTCAGACGATTGACACCTGGCTGGCTTGGTATTTGCAGAAACCTGGGAAAAG
CCCTCAGCTGCTGATCTACGCAGCCACCAATTTAGCCGATGGCGTCCCTTCTAGATTTAGCGGC
TCGGGATCCGGCACCGATTTTAGCTTTACAATTTCATCTCTACAGCCTGAGGACTTCGCCACCT
ATTATTGTCAACAAGTATATAGCAGCCCTTTCACCTTCGGCCAAGGCACCAAGTTAGAGATCAA
GGGCGGTGGCGGCTCCGGGGGCGGGGGAAGTGGAGGTGGGGGGTCACAGATTCAGCTGGTTCAA
AGCGGCGGCGAGGTGAAGAAACCTGGCGCCTCGGTGAGAGTTAGCTGCAAAGCTAGCGGATATT
CGTTTACTGACTACAACGTGTATTGGGTGAGGCAGAGCCCGGGCAAGGGACTGGAGTGGATTGG
GTACATTGACCCCTACAACGGCATAACCATATATGATCAGAACTTCAAGGGCAAAGCCACGCTG
ACCGTGGATAAAAGCACAAGCACCGCTTATATGGAGTTGAGCTCCCTCCGTAGCGAAGACACGG
CAGTATACTTTTGCGCACGGGATGTGACCACGGCCCTAGATTTCTGGGGACAAGGGACAACCGT
TACTGTTAGTTCC
(Linker)
GGCGGTGGGGGCTCG
(Human IL-15Rα sushi domain)
ATCACGTGCCCTCCCCCCATGTCCGTGGAACACGCAGACATCTGGGTCAAGAGCTACAGCTTGT
ACTCCAGGGAGCGGTACATTTGTAACTCTGGTTTCAAGCGTAAAGCCGGCACGTCCAGCCTGAC
GGAGTGCGTGTTGAACAAGGCCACGAATGTCGCCCACTGGACAACCCCCAGTCTCAAATGCATT
AGAGACCCTGCCCTGGTTCACCAAAGG
(Anti-human CD3scFv)
GATATTCAGATGACGCAGTCGCCAAGCTCACTCTCGGCCTCCGTAGGAGACAGAGTTACCATCA
CCTGCAGAGCTAGCCAAGACATCAGAAACTACCTGAACTGGTATCAGCAGAAGCCTGGCAAGGC
CCCTAAGCTCCTGATCTACTACACGAGCAGACTTGAGAGCGGGGTACCATCCCGCTTCAGCGGA
TCTGGAAGCGGCACAGACTACACCCTGACCATCTCTAGTCTGCAGCCTGAGGACTTCGCCACTT
ACTATTGTCAGCAAGGCAACACCCTGCCTTGGACCTTTGGCCAAGGCACCAAAGTTGAGATAAA
GGGAGGGGGAGGGAGCGGTGGCGGAGGCTCGGGTGGGGGTGGCTCAGAAGTACAGCTGGTTGAG
TCCGGCGGTGGTCTGGTGCAGCCTGGCGGAAGCCTGAGACTGAGCTGCGCCGCAAGCGGCTATA
GCTTCACTGGCTATACCATGAACTGGGTGCGCCAAGCCCCTGGCAAGGGCCTGGAATGGGTGGC
CCTGATCAACCCTTACAAGGGCGTGAGCACCTACAATCAGAAGTTCAAGGACAGATTCACCATC
AGCGTGGACAAGAGCAAGAACACCGCCTACCTGCAGATGAACAGCCTGAGAGCCGAGGACACTG
CGGTCTATTACTGTGCTAGAAGCGGCTACTACGGCGACAGCGACTGGTACTTCGACGTGTGGGG
GCAAGGCACCTTGGTAACGGTAAGCTCC

The amino acid sequence of the anti-TFscFv/IL-15RαSu/anti-CD3scFv fusion protein (including the mouse immunoglobulin H chain leader sequence) is as follows (SEQ ID NO: 175):

(Signal peptide)
MGWSCIILFLVATATGVHS
(Humanized anti-human TFscFv)
DIQMTQSPASLSASVGDRVTITCLASQTIDTWLAWYLQKPGKSPQLLIYAATNLADGVPSRFSG
SGSGTDFSFTISSLQPEDFATYYCQQVYSSPFTFGQGTKLEIKGGGGSGGGGSGGGGSQIQLVQ
SGGEVKKPGASVRVSCKASGYSFTDYNVYWVRQSPGKGLEWIGYIDPYNGITIYDQNFKGKATL
TVDKSTSTAYMELSSLRSEDTAVYFCARDVTTALDFWGQGTTVTVSS
(Linker)
GGGGS
(Human IL-15Rα sushi domain)
ITCPPPMSVEHADIWVKSYSLYSRERYICNSGFKRKAGTSSLTECVLNKATNVAHWTTPSLKCI
RDPALVHQR
(Anti-human CD3scFv)
DIQMTQSPSSLSASVGDRVTITCRASQDIRNYLNWYQQKPGKAPKLLIYYTSRLESGVPSRFSG
SGSGTDYTLTISSLQPEDFATYYCQQGNTLPWTFGQGTKVEIKGGGGSGGGGSGGGGSEVQLVE
SGGGLVQPGGSLRLSCAASGYSFTGYTMNWVRQAPGKGLEWVALINPYKGVSTYNQKFKDRFTI
SVDKSKNTAYLQMNSLRAEDTAVYYCARSGYYGDSDWYFDVWGQGTLVTVSS

Constructs were also made linking the human TGF-beta type II receptor sequence to the N-terminus of human TRBC1 following with the N-terminus coding region of IL-15 synthesized by Genewiz. The nucleic acid and protein sequences of a construct comprising human TGF-beta type II receptor linked to the N-terminus of human TRBC1 following with the N-terminus coding region of IL-15 are shown below.

The nucleic acid sequence of the TGFR/hTRβC1/IL-15 construct (including the N-terminal native G. princeps signal peptide sequence) is as follows (SEQ ID NO: 128):

(N-terminal native G. princeps signal peptide)
ATGGGCGTGAAGGTGCTGTTCGCCCTGATCTGCATCGCCGTGGCCGAGGCC
(Human TGF-beta type II receptor)
ATTCCTCCTCACGTGCAGAAAAGCGTGAACAACGACATGATAGTGACCGACAATAACGGCGCGG
TCAAGTTCCCACAGCTCTGCAAATTTTGCGACGTGAGATTTAGTACCTGCGACAATCAGAAATC
CTGCATGAGCAATTGTAGCATCACTAGCATCTGTGAAAAGCCCCAAGAGGTCTGCGTAGCAGTG
TGGAGAAAAAATGACGAGAACATCACACTAGAGACCGTGTGCCATGACCCTAAGCTGCCTTATC
ATGATTTCATCCTTGAAGATGCTGCTAGCCCTAAGTGCATTATGAAAGAGAAAAAGAAGCCTGG
CGAAACCTTTTTCATGTGTAGCTGTAGTAGCGACGAGTGCAACGATAACATCATCTTCTCGGAG
GAGTATAATACATCGAATCCTGATGGCGGCGGCGGCAGCATCCCTCCTCATGTTCAGAAGAGCG
TGAATAACGACATGATTGTGACGGATAACAATGGCGCCGTAAAGTTCCCTCAGCTGTGTAAGTT
CTGCGATGTGCGATTTAGCACCTGCGACAATCAGAAGAGCTGTATGTCCAATTGTTCAATCACA
AGCATCTGTGAGAAGCCTCAAGAGGTGTGCGTGGCCGTTTGGAGAAAGAACGACGAGAACATTA
CCCTTGAGACCGTATGCCACGATCCTAAGCTGCCGTATCACGACTTCATACTGGAGGATGCCGC
TAGCCCTAAATGCATCATGAAAGAAAAAAAAAAGCCTGGTGAGACATTTTTTATGTGTAGCTGC
TCGTCTGACGAGTGCAATGACAATATCATTTTCAGCGAAGAATATAACACCTCAAACCCTGAC
(Human TRBC1)
GAGGACCTGAACAAGGTGTTCCCACCCGAGGTCGCTGTGTTTGAGCCATCAGAAGCAGAGATCT
CCCACACCCAAAAGGCCACACTGGTGTGCCTGGCCACAGGCTTCTTCCCCGACCACGTGGAGCT
GAGCTGGTGGGTGAATGGGAAGGAGGTGCACAGTGGGGTCAGCACGGACCCGCAGCCCCTCAAG
GAGCAGCCCGCCCTCAATGACTCCAGATACTGCCTGAGCAGCCGCCTGAGGGTCTCGGCCACCT
TCTGGCAGAACCCCCGCAACCACTTCCGCTGTCAAGTCCAGTTCTACGGGCTCTCGGAGAATGA
CGAGTGGACCCAGGATAGGGCCAAACCCGTCACCCAGATCGTCAGCGCCGAGGCCTGGGGTAGA
GCAGAC
(Human IL-15)
AACTGGGTGAACGTGATCAGCGACCTGAAGAAGATCGAGGACCTGATTCAGAGCATGCACATCG
ACGCCACCCTGTACACCGAGAGCGACGTGCACCCTAGCTGCAAGGTGACCGCCATGAAGTGCTT
CCTGCTGGAGCTGCAAGTGATCAGCCTGGAGAGCGGCGACGCTAGCATCCACGACACCGTGGAG
AACCTGATCATCCTGGCCAACAACAGCCTGAGCAGCAACGGCAACGTGACCGAGAGCGGCTGCA
AGGAGTGCGAGGAGCTGGAGGAGAAGAACATCAAGGAGTTCCTGCAGAGCTTCGTGCACATCGT
GCAGATGTTCATCAACACAAGC

The amino acid sequence of the TGFR/hTRβC1/IL-15 fusion protein (including the N-terminal native G. princeps signal peptide sequence) is as follows (SEQ ID NO: 193):

(N-terminal native G. princeps signal peptide)
MGVKVLFALICIAVAEA
(Human TGF-beta type II receptor)
IPPHVQKSVNNDMIVTDNNGAVKFPQLCKFCDVRFSTCDNQKSCMSNCSITSICEKPQEVCVAV
WRKNDENITLETVCHDPKLPYHDFILEDAASPKCIMKEKKKPGETFFMCSCSSDECNDNIIFSE
EYNTSNPDGGGGSIPPHVQKSVNNDMIVTDNNGAVKFPQLCKFCDVRFSTCDNQKSCMSNCSIT
SICEKPQEVCVAVWRKNDENITLETVCHDPKLPYHDFILEDAASPKCIMKEKKKPGETFFMCSC
SSDECNDNIIFSEEYNTSNPD
(Human TRBC1)
EDLNKVFPPEVAVFEPSEAEISHTQKATLVCLATGFFPDHVELSWWVNGKEVHSGVSTDPQPLK
EQPALNDSRYCLSSRLRVSATFWQNPRNHFRCQVQFYGLSENDEWTQDRAKPVTQIVSAEAWGR
AD
(Human IL-15)
NWVNVISDLKKIEDLIQSMHIDATLYTESDVHPSCKVTAMKCFLLELQVISLESGDASIHDTVE
NLIILANNSLSSNGNVTESGCKECEELEEKNIKEFLQSFVHIVQMFINTS

In some cases, the leader peptide is cleaved from the intact polypeptide to generate the mature form that may be soluble or secreted.

The anti-TFscFv/IL-15RαSu/anti-CD3scFv and TGFR/hTRβC1/IL-15 constructs were cloned into a modified retrovirus expression vectors pMSGV-1 as described previously (Hughes M S, Yu Y Y, Dudley M E, Zheng Z, Robbins P F, Li Y, et al. Transfer of a TCR gene derived from a patient with a marked antitumor response conveys highly active T-cell effector functions. Hum Gene Ther 2005; 16:457-72.), and the expression vectors were transfected into CHO-K1 cells. Co-expression of the two constructs in CHO-K1 cells allowed for formation and secretion of a soluble TGFβRII/hTRβC1/IL-15:anti-TFscFv/IL-15RαSu/anti-CD3scFv. The TGFβRII/hTRβC1/IL-15:anti-TF scFv/IL-15RαSu/anti-CD3 scFv protein was purified from CHO cell culture supernatant by an anti-TRBC1 antibody (BF1) affinity chromatography and size exclusion chromatography resulting in soluble (non-aggregated) protein complexes consisting of anti-TFscFv/IL-15RαSu/anti-CD3scFv: TGFR/hTRβC1/IL-15 fusion proteins (FIG. 52B).

### Example 32: Characterization of Individual Components of TGFβRII/hTRβC1/IL-15:Anti-TFscFv/IL-15RαSu/anti-CD3scFv and IL-7/hTRpC1/IL-15:anti-TFscFv/IL-15RαSu/anti-CD3scFv

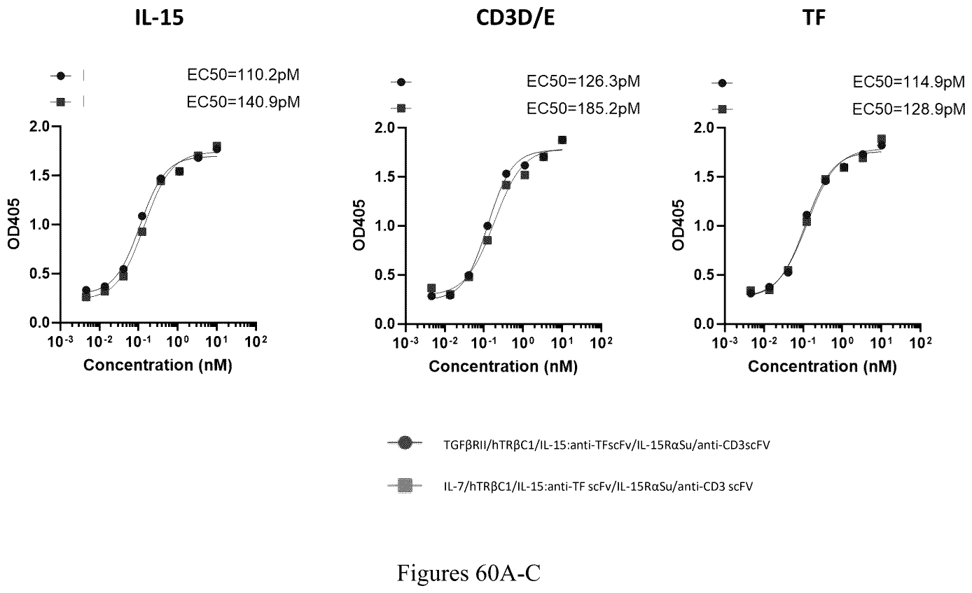

Binding ability of individual components of TGFβRII/hTRβC1/IL-15:anti-TF scFv/IL-15RαSu/anti-CD3 scFv and IL-7/hTRβC1/IL-15:anti-TFscFv/IL-15RαSu/anti-CD3scFv. Increasing concentrations of TGFβRII/hTRβC1/IL-15:anti-TF scFv/IL-15RαSu/anti-CD3 scFv or IL-7/hTRβC1/IL-15:anti-TFscFv/IL-15RαSu/anti-CD3scFv were captured with BF1 (5 μg/mL). Interactions of biotinylated anti-IL-15, CD3D/E, and TF with TGFβRII/hTRβC1/IL-15:anti-TF scFv/IL-15RαSu/anti-CD3 scFv or IL-7/hTRβC1/IL-15:anti-TFscFv/IL-15RαSu/anti-CD3scFv were detected by HRP-SA. ABTS (2,2′-azino-bis (3-ethylbenzothiazoline-6-sulphonic acid) (Surmondics) was used as substrate of HRP. The OD405 was measured with a spectrophotometer. See FIGS. 60A-C. The data demonstrated that IL-15 levels, TF binding, and CD3D/E binding ability were same between IL-7/hTRβC1/IL-15:anti-TFscFv/IL-15RαSu/anti-CD3scFv and TGFβRII/hTRβC1/IL-15:anti-TFscFv/IL-15RαSu/anti-CD3scFv.

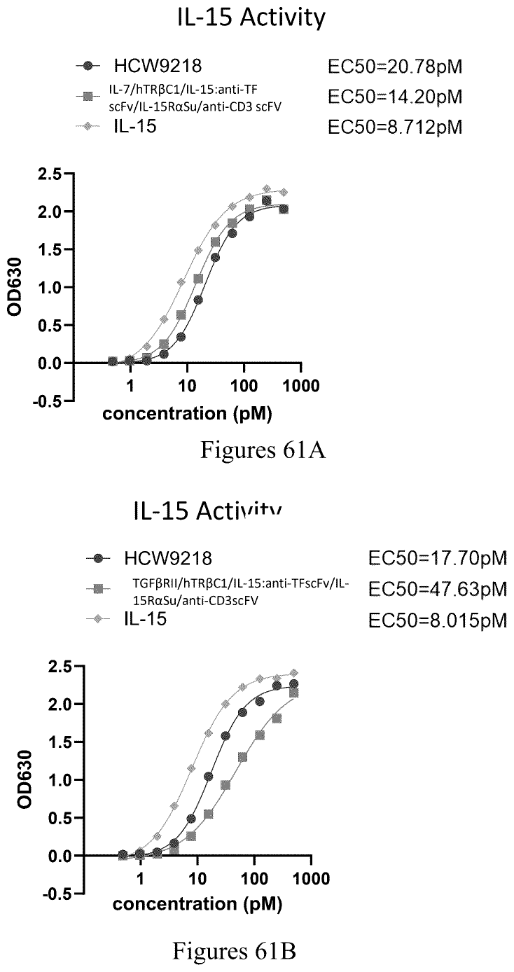

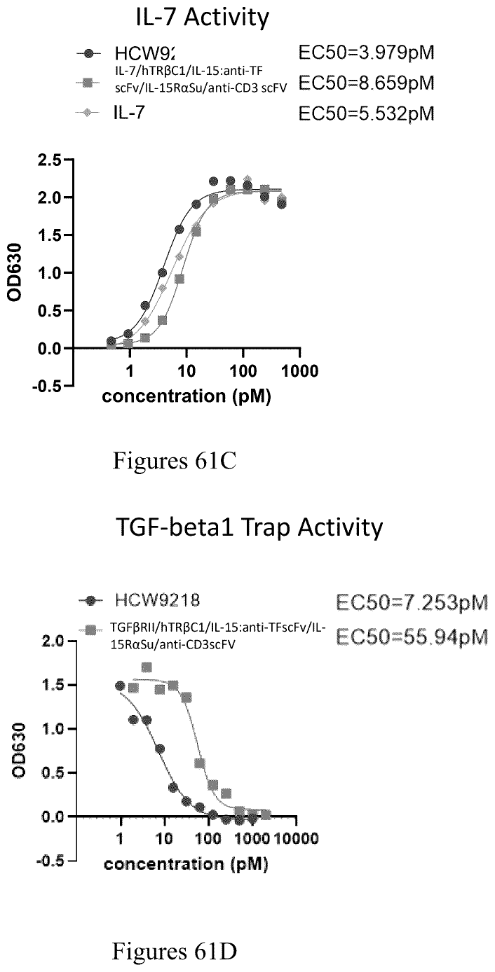

Activity of individual component of TGFβRII/hTRβC1/IL-15:anti-TF scFv/IL-15RαSu/anti-CD3scFv and IL-7/hTRβC1/IL-15:anti-TFscFv/IL-15RαSu/anti-CD3scFv. HEK-Blue™ CD122/CD132 cells (Invivogen) were stimulated with increasing concentrations of HCW9218, IL-15, IL-7/hTRβC1/IL-15:anti-TFscFv/IL-15RαSu/anti-CD3scFv, or TGFβRII/hTRβC1/IL-15:anti-TFscFv/IL-15RαSu/anti-CD3scFv. HEK-Blue™ IL-7 cells (Invivogen) were stimulated with increasing concentrations of HCW9206, IL-7, or IL-7/hTRβC1/IL-15:anti-TFscFv/IL-15RαSu/anti-CD3scFv. After overnight incubation, the STAT5 response was determined using QUANTIBlue™ Solution, a SEAP detection reagent. The optical density (OD) at 630 nm is shown as IL-15 or IL-7 activity. See FIGS. 61A-C.

HEK-Blue™ TGF-β cells (Cat #hkb-tgfb, Invivogen) were stimulated with TGF-β1 at 1.25 ng/ml (50pM) in the presence of increasing concentrations of HCW9218 or TGFβRII/hTRβC1/IL-15:anti-TF scFv/IL-15RαSu/anti-CD3 scFv. After overnight incubation, the TGF-β1/Smad signal pathway response was determined using QUANTI-Blue™ Solution, a SEAP detection reagent. The optical density (OD) at 630 nm is shown as TGF-01 trap activity. See FIGS. 61A-D. FIGS. 61A and B show IL-15 activity of the IL-15 components of IL-7/hTRβC1/IL-15:anti-TFscFv/IL-15RαSu/anti-CD3scFv and TGFβRII/hTRβC1/IL-15:anti-TF scFv/IL-15RαSu/anti-CD3 scFv. The data also shows the activity of the IL-7 component of IL-7/hTRβC1/IL-15:anti-TFscFv/IL-15RαSu/anti-CD3scFv and the TGF-01 trap activity of the TGFR component of TGFβRII/hTRβC1/IL-15:anti-TF scFv/IL-15RαSu/anti-CD3scFv.

### Example 33: Evaluation of Cytotoxic Effects on AsPC-1 and MIA Cell Lines by Human PBMCs Activated Via T-Cell Engagers

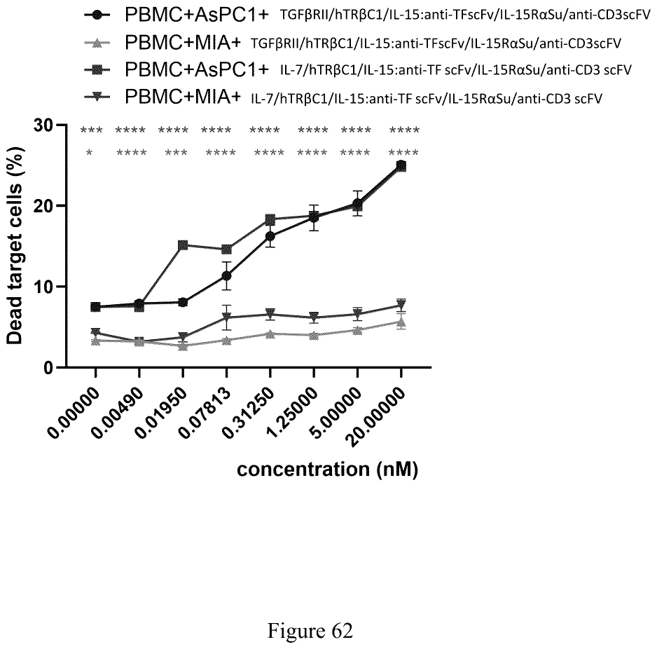

CellTrace Violet kit (Invitrogen) labeled AsPC-1 (TF+) or MIA-Pa-Ca-2 (TF−) cells (1.2×10⁴ cells/well) were incubated with TGFβRII/hTRβC1/IL-15:anti-TF scFv/IL-15RαSu/anti-CD3 scFv or IL-7/hTRβC1/IL-15:anti-TFscFv/IL-15RαSu/anti-CD3scFv (0-20 nM 14 dilution) for 30 min and then mixed with PBMCs from 3 donors (1.2×10⁵ cells/well), in a flat-bottom 96-well plate (Corning). After 20 h incubation, effector cells and supernatant were removed, target cells were trypsinized and resuspended in complete-medium with Propidium iodide (PI, ex/em=488/610) at 2 μg/mL and analyzed with FACSCelestra. The percentage of target cell killing were evaluated through the appearance of PI+ and CellTrace Violet+ dead cells. See FIG. 62.

This experiment demonstrated that IL-7/hTRβC1/IL-15:anti-TFscFv/IL-15RαSu/anti-CD3scFv and TGFβRII/hTRβC1/IL-15:anti-TF scFv/IL-15RαSu/anti-CD3 scFv mediated specific cytotoxic effects of PBMCs on tissue factor positive AsPC-1 pancreatic cancer cells, while these proteins mediated less cytotoxicity against TF-MIA-PaCa2 pancreatic cancer cells.

### Example 34: TCE-Mediated CD8⁺ T Cell Activation in Human PBMCs

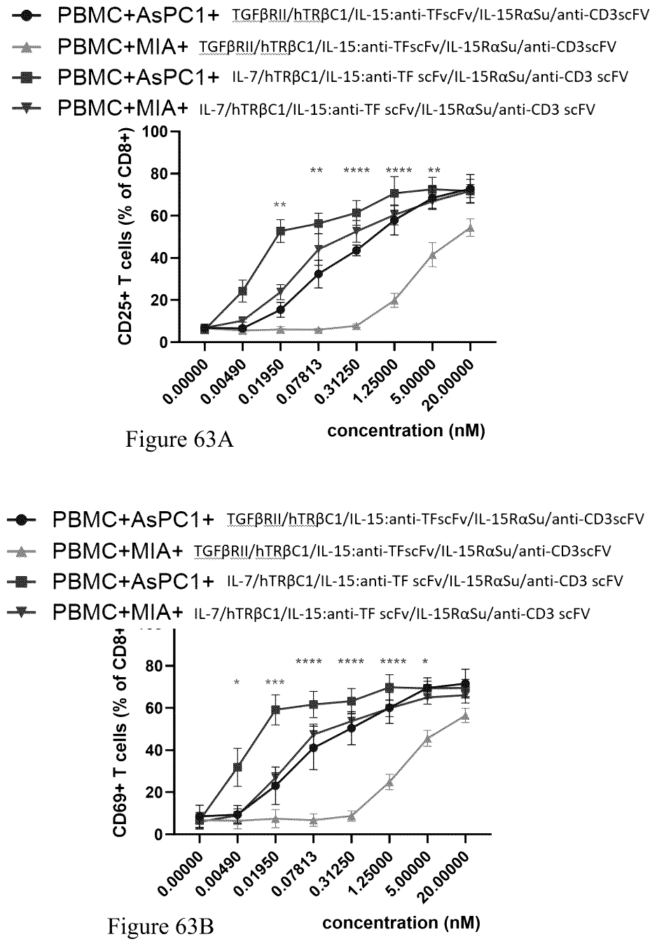

AsPC-1 (TF+) or MIA-Pa-Ca-2 (TF−) cells (1.2×10⁴ cells/well) were seeded in a flat-bottom 96-well plate (Corning) and incubated in a CO₂ incubator at 37° C. overnight. The next day, the supernatant was removed and TGFβRII/hTRβC1/IL-15:anti-TF scFv/IL-15RαSu/anti-CD3 scFv or IL-7/hTRβC1/IL-15:anti-TFscFv/IL-15RαSu/anti-CD3scFv (0-20 nM 14 dilution) was added for 30 min, and then mixed with PBMCs from 3 donors (1.2×10⁵ cells/well). After 1-day incubation, effector cells are first stained with BV510-live or dead (ThermoFisher) and then washed once using FACS buffer and stained with PE-labeled anti-CD3 (BD Biosciences), APC/Cy7-labeled anti-CD8 (BD Biosciences), AF488-labeled anti-CD4 (BioLegend), APC-anti-CD25 (BioLegend), and PerCPCy5.5-labeled anti-CD69 antibodies (BioLegend) under manufacturer's instruction. After washing twice with FACS buffer, effector cells were analyzed on FACSCelestra. The percentages of CD69⁺ or CD25⁺ cells within CD8⁺ T cell populations were calculated. See FIGS. 63A-B.

This experiment demonstrated that IL-7/hTRβC1/IL-15:anti-TFscFv/IL-15RαSu/anti-CD3scFv and TGFβRII/hTRβC1/IL-15:anti-TF scFv/IL-15RαSu/anti-CD3 scFv mediated a robust specific increase in CD69+ or CD25+CD8⁺ T cells when incubated with tissue factor positive AsPC-1 pancreatic cancer cells, while these proteins mediated a lower increase in CD69+ or CD25+CD8⁺ T cells when incubated with TF-MIA-PaCa2 pancreatic cancer cells.

### Example 35: TCE-Mediated CD4⁺ T Cell Activation in Human PBMCs

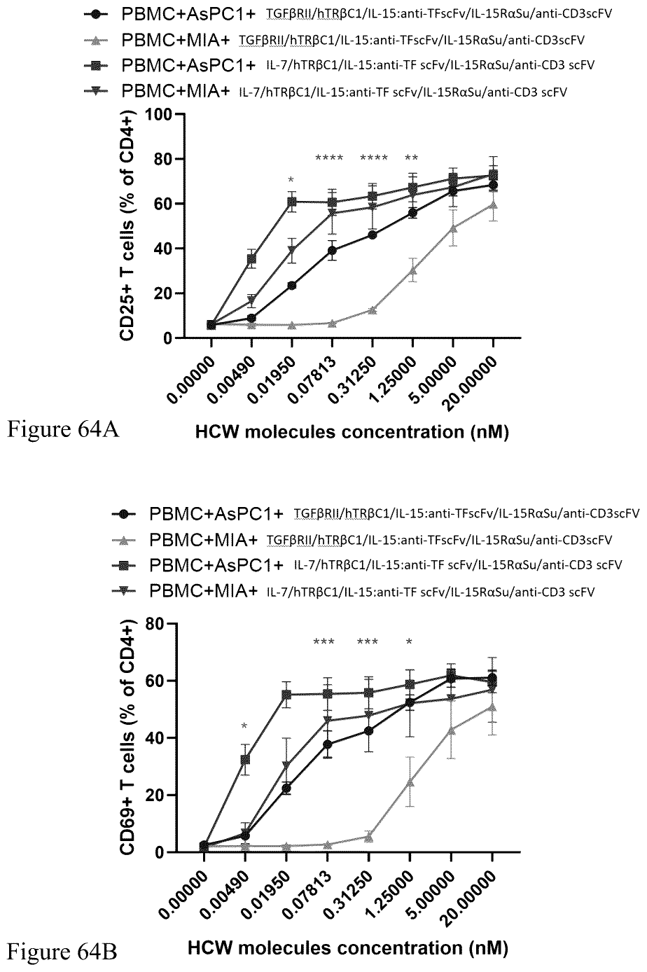

AsPC-1 (TF+) or MIA-Pa-Ca-2 (TF−) cells (1.2×10⁴ cells/well) were seeded in a flat-bottom 96-well plate (Corning) and incubated in a CO₂ incubator at 37° C. overnight. The next day, the supernatant was removed and TGFβRII/hTRβC1/IL-15:anti-TF scFv/IL-15RαSu/anti-CD3 scFv or IL-7/hTRβC1/IL-15:anti-TFscFv/IL-15RαSu/anti-CD3scFv (0-20 nM 14 dilution) was added for 30 min, and then mixed with PBMCs from 3 donors (1.2×10⁵ cells/well) respectively. After 1-day incubation, effector cells are first stained with BV510-live or dead (ThermoFisher) and then washed once using FACS buffer and stained with PE-labeled anti-CD3 (BD Biosciences), APC/Cy7-labeled anti-CD8 (BD Biosciences), AF488-labeled anti-CD4 (BioLegend), APC-anti-CD25 (BioLegend), and PerCPCy5.5-labeled anti-CD69 antibodies (BioLegend) under manufacturer's instruction. After washing twice with FACS buffer, effector cells were analyzed on FACSCelestra. The percentages of CD69⁺ or CD25⁺ cells within CD4⁺ T cell populations were calculated. See FIGS. 64A-B.

This experiment demonstrated that IL-7/hTRβC1/IL-15:anti-TFscFv/IL-15RαSu/anti-CD3scFv and TGFβRII/hTRβC1/IL-15:anti-TF scFv/IL-15RαSu/anti-CD3 scFv mediated a robust specific increase in CD69⁺ or CD25⁺ CD4⁺ T cells when incubated with tissue factor positive AsPC-1 pancreatic cancer cells, while these proteins mediated a lower increase in CD69⁺ or CD25⁺ CD4⁺ T cells when incubated with TF-MIA-PaCa2 pancreatic cancer cells.

### Example 36: Cytokine Profiling of T-Cell Engagers IL-7/hTRpC1/IL-15:Anti-TFscFv/IL-15RαSu/anti-CD3scFv/11-018b Using CBA Kit in Human PBMC

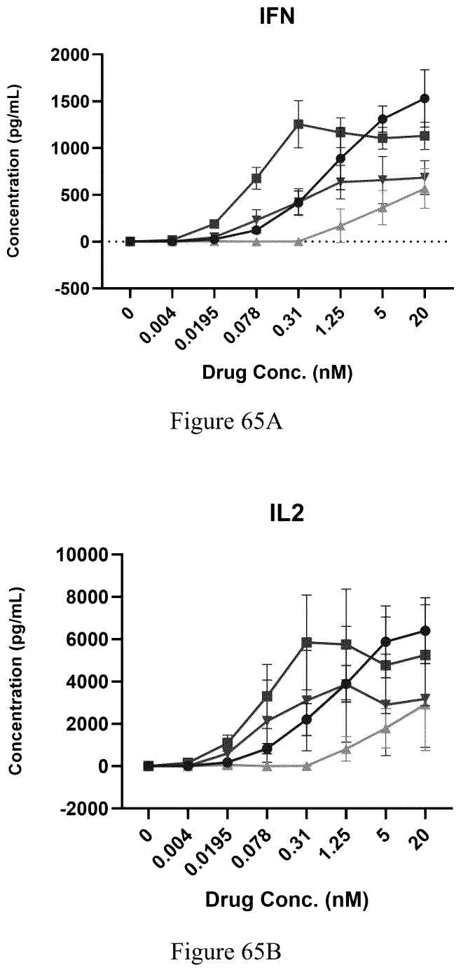

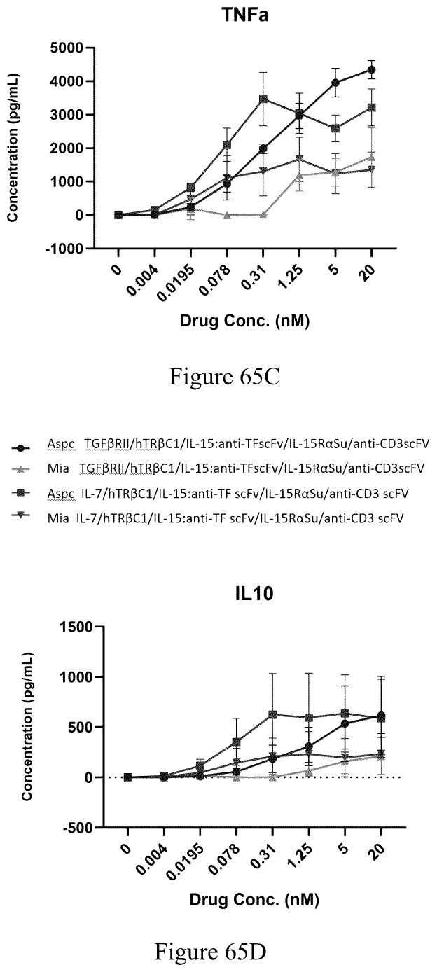

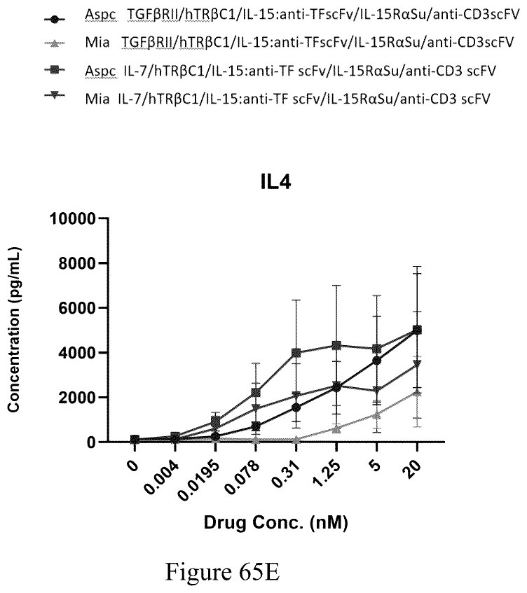

AsPC-1 (TF+) or MIA-Pa-Ca-2 (TF−) cells (1.2×10⁴ cells/well) were seeded in a flat-bottom 96-well plate (Corning), TGFβRII/hTRβC1/IL-15:anti-TF scFv/IL-15RαSu/anti-CD3 scFv or IL-7/hTRβC1/IL-15:anti-TFscFv/IL-15RαSu/anti-CD3scFv (0-20 nM 14 dilution) was added for 30 min, and then mixed with PBMC from 3 donors (1.2×10⁵ cells/well). After overnight incubation, cell supernatant was collected and analyzed for different cytokines utilizing human CBA Kit (BD Bioscience). See FIGS. 65A-E. This study demonstrated TGFβRII/hTRβC1/IL-15:anti-TF scFv/IL-15RαSu/anti-CD3 scFv had more specific PMBC activation based on cytokine profiles while engaging TF+ AsPC-1 cells vs. TF-MIA-PaCa2 cell compared with IL-7/hTRβC1/IL-15:anti-TFscFv/IL-15RαSu/anti-CD3scFv.

### Example 37: TCE-Mediated CD8⁺ T Cell Activation in CD2⁺ Human T Cells

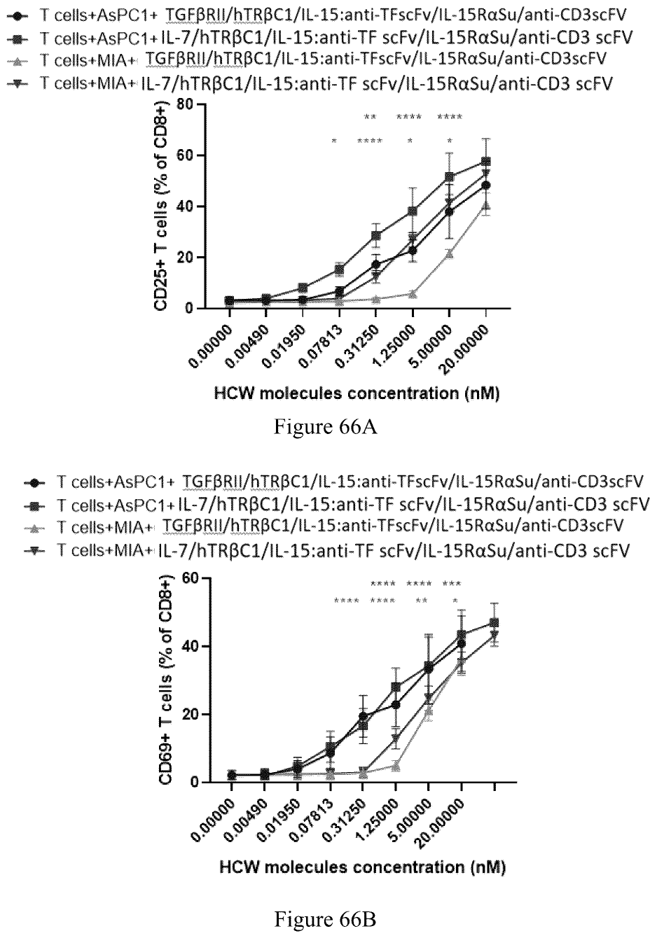

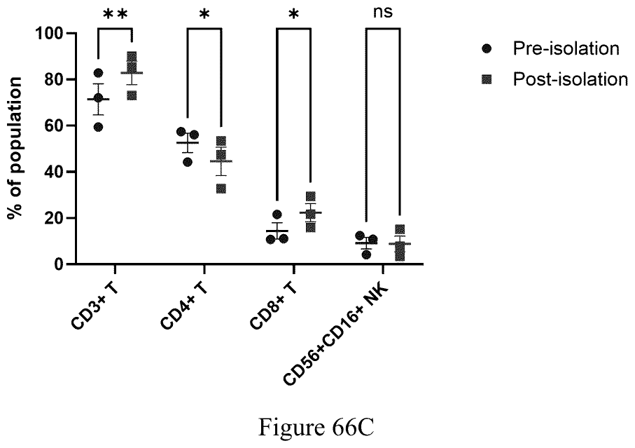

AsPC-1 (TF+) or MIA-Pa-Ca-2 (TF−) cells (1.2×10⁴ cells/well) were seeded in a flat-bottom 96-well plate (Corning), TGFβRII/hTRβC1/IL-15:anti-TF scFv/IL-15RαSu/anti-CD3 scFv or IL-7/hTRβC1/IL-15:anti-TFscFv/IL-15RαSu/anti-CD3scFv (0-20 nM 14 dilution) was added for 30 min, and then mixed with CD2+ bead-purified T cells from 3 donors (1.2×10⁵ cells/well). After 1-day incubation, effector cells were first stained with BV510-live or dead (ThermoFisher) and then washed once using FACS buffer and stained with PE-labeled anti-CD3 (BD Biosciences), APC/Cy7-labeled anti-CD8 (BD Biosciences), AF488-labeled anti-CD4 (BioLegend), APC-anti-CD25 (BioLegend), and PerCPCy5.5-labeled anti-CD69 antibodies (BioLegend) under manufacturer's instruction. After washing twice with FACS buffer, effector cells were analyzed on FACSCelestra. The percentages of CD69⁺ or CD25⁺ cells within CD8⁺ T cell populations were calculated. See FIGS. 66A-C.

This experiment demonstrated that IL-7/hTRβC1/IL-15:anti-TFscFv/IL-15RαSu/anti-CD3scFv and TGFβRII/hTRβC1/IL-15:anti-TF scFv/IL-15RαSu/anti-CD3 scFv mediated a robust specific increase in CD69⁺ or CD25+CD8⁺ T cells when incubated with tissue factor positive AsPC-1 pancreatic cancer cells, while these proteins mediated a lower increase in CD69⁺ or CD25⁺ CD8⁺ T cells when incubated with TF-MIA-PaCa2 pancreatic cancer cells.

### Example 38: TCE-Mediated CD4⁺ T Cell Activation in CD2⁺ Human T Cells

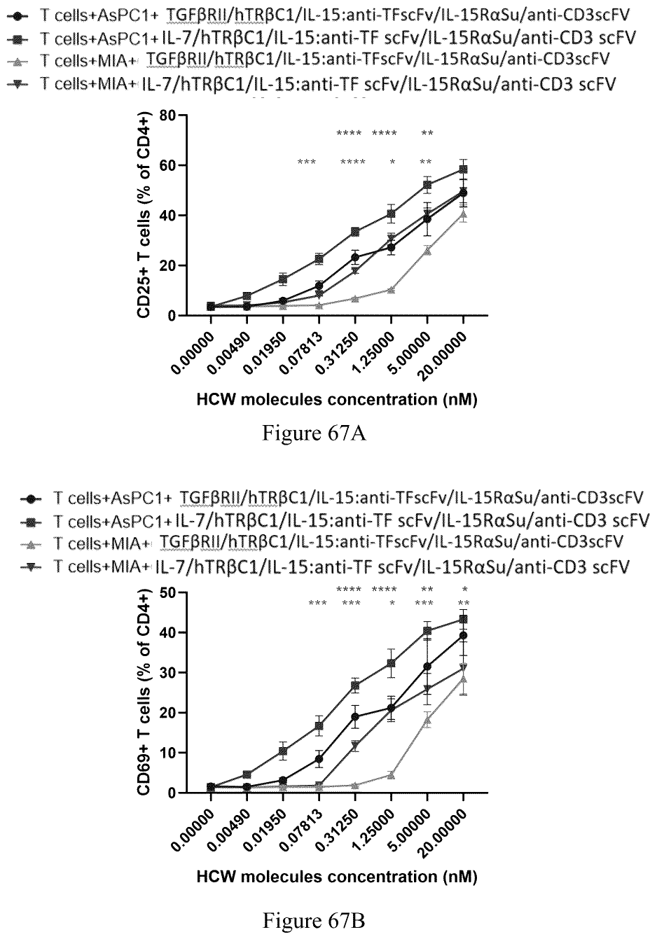

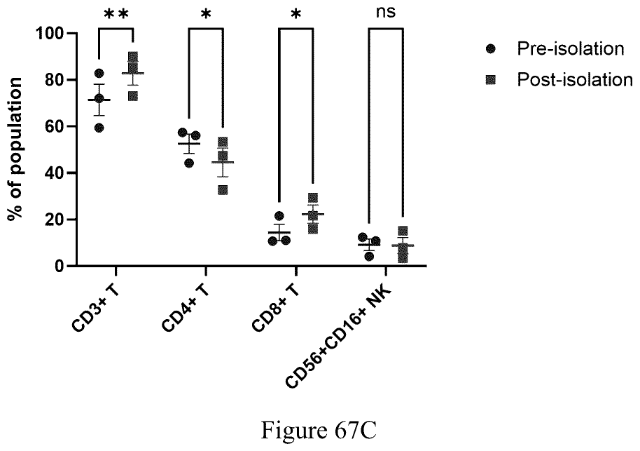

AsPC-1 (TF+) or MIA-Pa-Ca-2 (TF−) cells (1.2×10⁴ cells/well) were seeded in a flat-bottom 96-well plate (Corning) and TGFβRII/hTRβC1/IL-15:anti-TF scFv/IL-15RαSu/anti-CD3 scFv or IL-7/hTRβC1/IL-15:anti-TFscFv/IL-15RαSu/anti-CD3scFv (0-20 nM 14 dilution) was added for 30 min, and then mixed with CD2+ bead-purified T cells from 3 donors (1.2×10⁵ cells/well). After 1-day incubation, effector cells were first stained with BV510-live or dead (ThermoFisher) and then washed once using FACS buffer and stained with PE-labeled anti-CD3 (BD Biosciences), APC/Cy7-labeled anti-CD8 (BD Biosciences), AF488-labeled anti-CD4 (BioLegend), APC-anti-CD25 (BioLegend), and PerCPCy5.5-labeled anti-CD69 antibodies (BioLegend) under manufacturer's instruction. After washing twice with FACS buffer, effector cells were analyzed on FACSCelestra. The percentages of CD69⁺ or CD25⁺ cells within the CD4⁺ T cell populations was calculated. See FIGS. 67A-C.

This experiment demonstrated that IL-7/hTRβC1/IL-15:anti-TFscFv/IL-15RαSu/anti-CD3scFv and TGFβRII/hTRβC1/IL-15:anti-TF scFv/IL-15RαSu/anti-CD3 scFv mediated a robust specific increase in CD69⁺ or CD25⁺ CD4⁺ T cells when incubated with tissue factor positive AsPC-1 pancreatic cancer cells, while these proteins mediated a lower increase in CD69⁺ or CD25⁺ CD4⁺ T cells when incubated with TF-MIA-PaCa2 pancreatic cancer cells.

### Example 39: Cytokine Profiling of T-Cell Engagers IL-7/hTRpC1/IL-15:Anti-TFscFv/IL-15RαSu/Anti-CD3scFv/11-018b Using CBA Kit in CD2⁺ Human T Cells

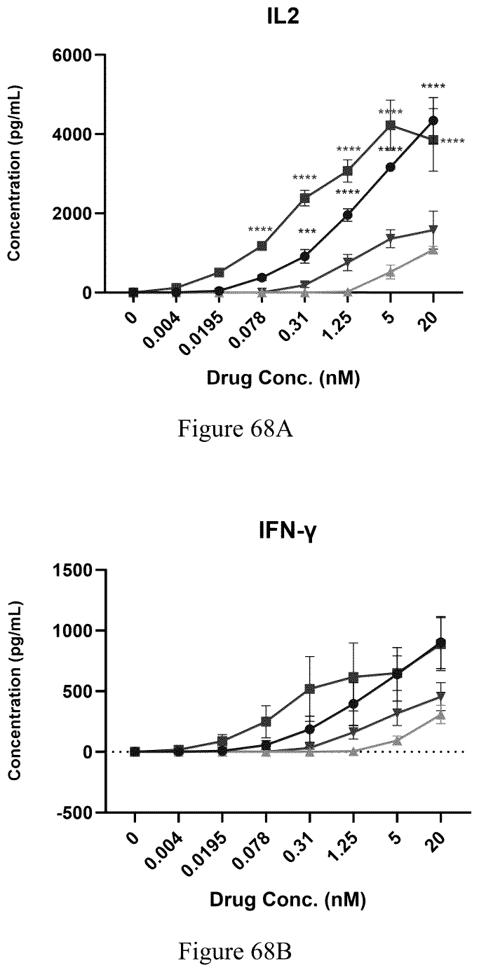

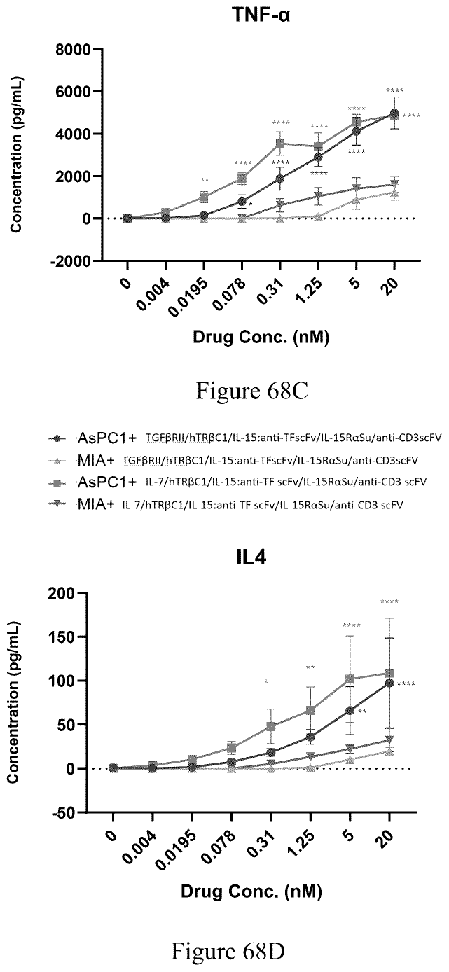

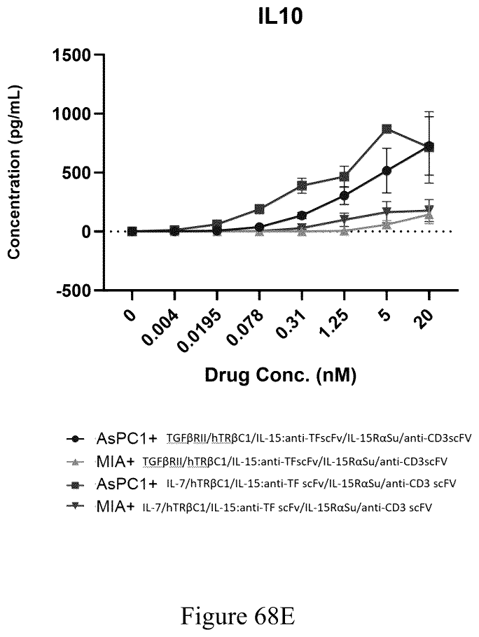

AsPC-1 (TF+) or MIA-Pa-Ca-2 (TF−) cells (1.2×10⁴ cells/well) were seeded in a flat-bottom 96-well plate (Corning) and TGFβRII/hTRβC1/IL-15:anti-TF scFv/IL-15RαSu/anti-CD3 scFv or IL-7/hTRβC1/IL-15:anti-TFscFv/IL-15RαSu/anti-CD3scFv (0-20 nM 14 dilution) was added for 30 min and then mixed with CD2⁺ bead-purified T cells from 3 donors (1.2×10⁵ cells/well). After overnight incubation, cell supernatant was collected and analyzed for different cytokines utilizing human CBA Kit (BD Bioscience). See FIGS. 68A-E.

This study demonstrated TGFβRII/hTRβC1/IL-15:anti-TF scFv/IL-15RαSu/anti-CD3 scFv had more specific T cell activation based on cytokine profiles while engaging TF+ AsPC-1 cells vs. TF-MIA-PaCa2 cell compared with IL-7/hTRβC1/IL-15:anti-TFscFv/IL-15RαSu/anti-CD3scFv.

### Example 40: IL-7/hTRpC1/IL-15:Anti-TFscFv/IL-15RαSu/Anti-CD3scFv and TGFβRII/hTRβC1/IL-15:Anti-TF scFv/IL-15RαSu/Anti-CD3scFv SCID Mouse Model

Multi-chain chimeric polypeptides were evaluated for anti-tumor efficacy using a SCID mouse model. ASPC tumor cells were co-mixed with hPBMCs and implanted into the mice. The animals were treated with nine doses of multi-chain chimeric polypeptides, administered at a dose of 1 mg/kg. Dosing occurred at intervals of every four days. Key endpoints measured included tumor growth, body weight, and overall survival of the mice.

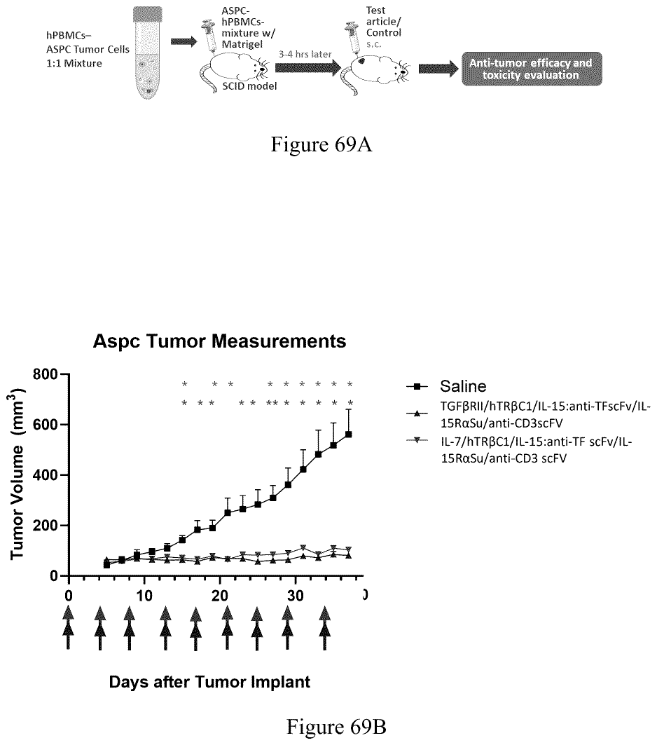

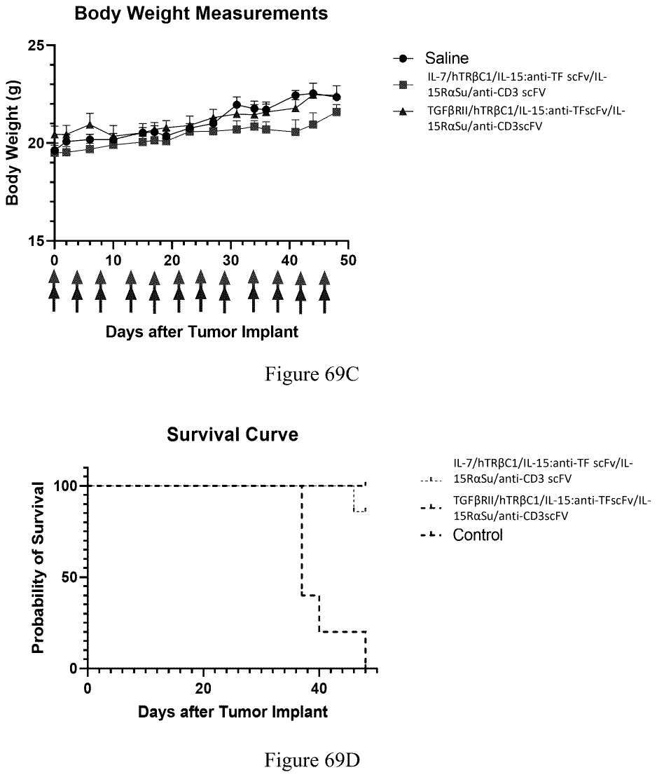

Impact on Survival and Tumor Control. Tumor volume and mouse body weight were regularly monitored to assess therapeutic efficacy and potential toxicity. Survival data were collected to evaluate the long-term benefits of the treatment. See FIGS. 69A-D.

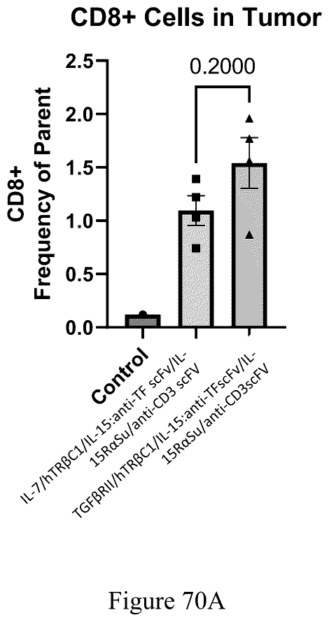

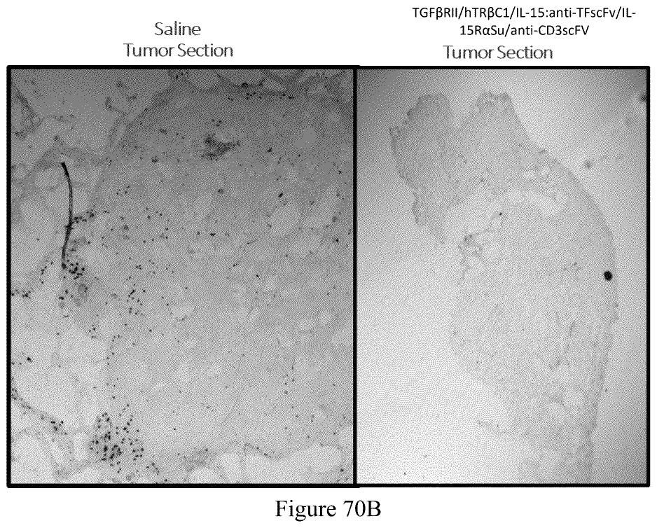

Immunohistology of Tumor Sections in TGFβRII/hTRβC1/IL-15:anti-TF scFv/IL-15RαSu anti-CD3 scFv or Control treated SCID Mouse. Mice treated with nine doses of TGFβRII/hTRβC1/IL-15:anti-TF scFv/IL-15RαSu/anti-CD3 scFv showed an increase in human CD8⁺ T cells within the tumor. Histological analysis also revealed that tissue factor expression was reduced in mice treated with TGFβRII/hTRβC1/IL-15:anti-TF scFv/IL-15RαSu/anti-CD3 scFv compared to control animals. See FIGS. 70A-B. TGFβRII/hTRβC1/IL-15:anti-TF scFv/IL-15RαSu/anti-CD3 scFv promoted significant tumor regression and complete response compared to IL-7/hTRβC1/IL-15:anti-TFscFv/IL-15RαSu/anti-CD3scFv or saline treated groups in human PBMC plus tumor cell co-grafting SCID mice model.

### Example 41: TGFβRII/hTRβC1/IL-15:Anti-TF scFv/IL-15RαSu/Anti-CD3 scFv Anti-Tumor Activity in NSG Mice

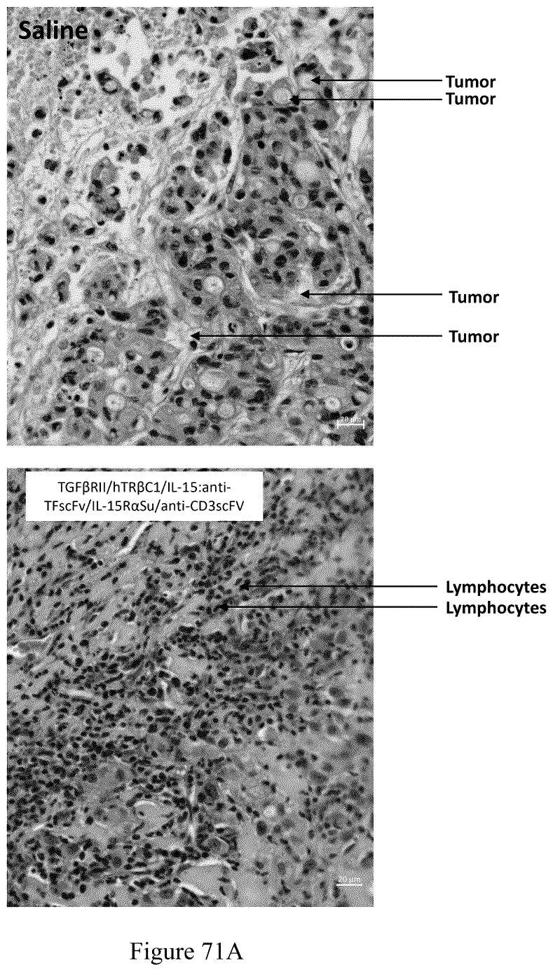

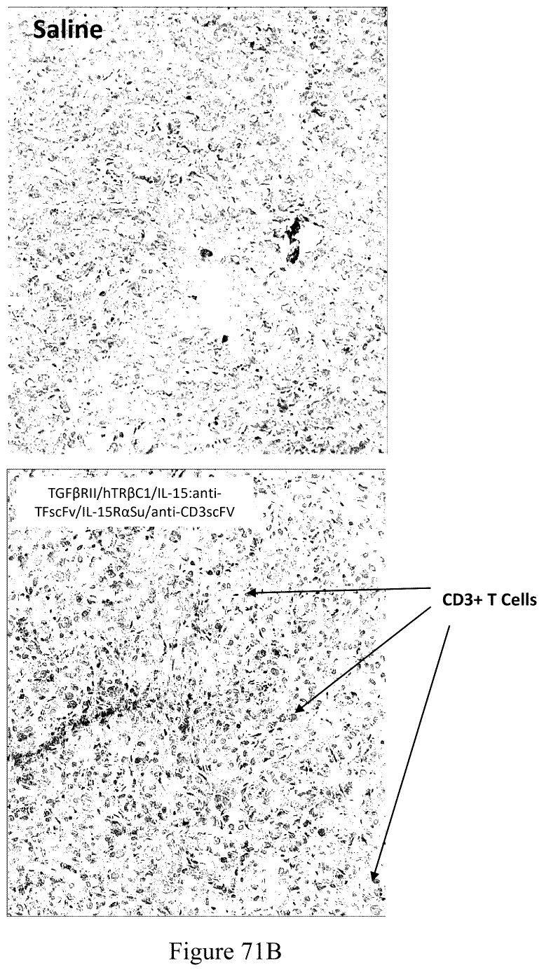

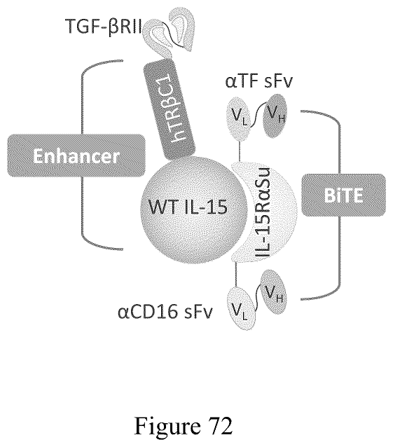

Pancreatic cancer infiltrated with lymphocytes in NSG mice treated with TGFβRII/hTRβC1/IL-15:anti-TF scFv/IL-15RαSu anti-CD3 scFv. 6-wk old NOD.Cg-Prkdc^{scid} Il2rg^{tm1Wjl}/SzJ (NSG) mice were injected subcutaneously with 2×10⁶ human AsPC-1 tumor cells on Day 0. Once tumor volume reached between 50-100 mm³, each mouse received an i.v. infusion of 20×10⁶ isolated human T cells (expanded with αCD3/αCD28 beads). Mice were received s.c. dose of Saline or TGFβRII/hTRβC1/IL-15:anti-TF scFv/IL-15RαSu/anti-CD3 scFv (1 mg/kg). Treatments were given every 5 days for a total of six doses. Mice were sacrificed on day 39 (3 days following last dose) and tumors were excised and formalin-fixed. Tumor issues were paraffin-embedded, sectioned, and Hematoxylin and Eosin (H&E) stained. See FIG. 71A.

Data shows H&E stained human AsPC-1 tumor sections from NSG mice left untreated (Saline) or treated with TGFβRII/hTRβC1/IL-15:anti-TF scFv/IL-15RαSu/anti-CD3 scFv. Increased numbers of lymphocytes were observed in the treated tumors (indicated by arrows) of TGFβRII/hTRβC1/IL-15:anti-TF scFv/IL-15RαSu/anti-CD3 scFv treated mice compared to saline treated controls.

Pancreatic cancer infiltrated with CD3⁺ T Cells in NSG mice treated with TGFβRII/hTRβC1/IL-15:anti-TF scFv IL-15RαSu anti-CD3 scFv. 6-wk old NOD.Cg-Prkdc^{scid} Il2rg^{tm1Wjl}/SzJ (NSG) mice were injected subcutaneously with 2×10⁶ human AsPC-1 tumor cells on Day 0. Once tumor volume reached between 50-100 mm³, each mouse received an i.v. infusion of 20×10⁶ isolated human T cells (expanded with αCD3/αCD28 beads). Mice were received s.c. dose of Saline or TGFβRII/hTRβC1/IL-15:anti-TF scFv/IL-15RαSu/anti-CD3 scFv (1 mg/kg). Treatments were given every 5 days for a total of six doses. Mice were sacrificed on day 39 (3 days following last dose) and tumors were excised and formalin-fixed. Tumor issues were paraffin-embedded, sectioned, and CD3⁺ T cells were stained using commercially available anti-human antibodies. See FIG. 71B.

Data showed higher numbers of infiltrated CD3⁺ T cells in tumors from mice treated with TGFβRII/hTRβC1/IL-15:anti-TF scFv/IL-15RαSu/anti-CD3 scFv compared to Saline treated mice. This showed that TGFβRII/hTRβC1/IL-15:anti-TF scFv/IL-15RαSu/anti-CD3 scFv is able to enhance infiltration of CD3⁺ T cells in tumor.

### Example 42: TGFβRII/hTRβC1/IL-15:anti-TFscFv/IL-15RαSu/anti-CD16a-scFv Induced Activation of Human NK Cells

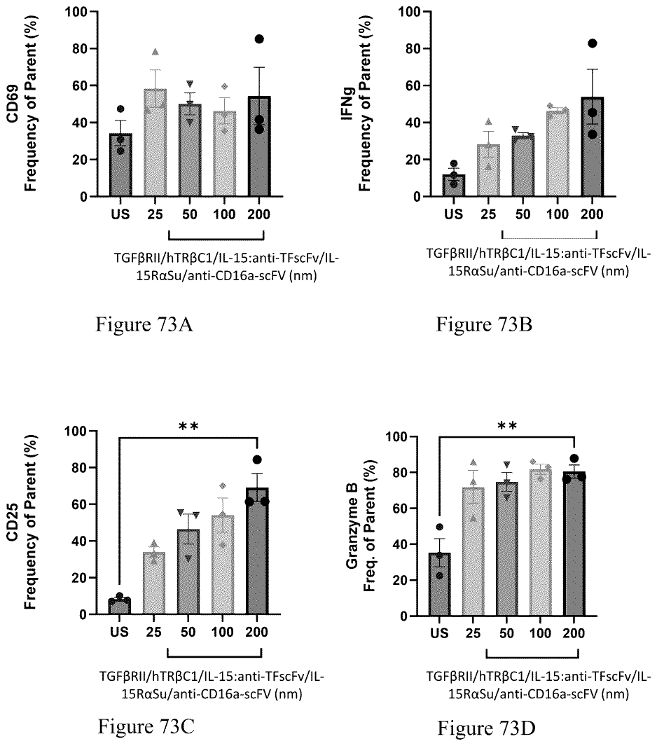

Frozen human peripheral blood mononuclear cells (PBMCs) were stimulated with either media alone or TGFβRII/hTRβC1/IL-15:anti-TFscFv/IL-15RαSu/anti-CD16a-scFv at different concentrations for overnight. BFA was added after 16 hrs. After a further additional 5 hrs, cells were harvested and stained for CD3, CD16, CD56, CD25, CD69, IFN-γ and Granzyme β for FACS analysis. NB: Cells were gated on CD16⁺ CD56⁺ cells. See FIGS. 73A-73D. The results show TGFβRII/hTRβC1/IL-15:anti-TFscFv/IL-15RαSu/anti-CD16a-scFv stimulation of human PBMCs and NK cells.

### Example 43: TGFβRII/hTRβC1/IL-15:anti-TFscFv/IL-15RαSu/anti-CD16a-scFv Mediated Induction of CD107a in Human PBMC

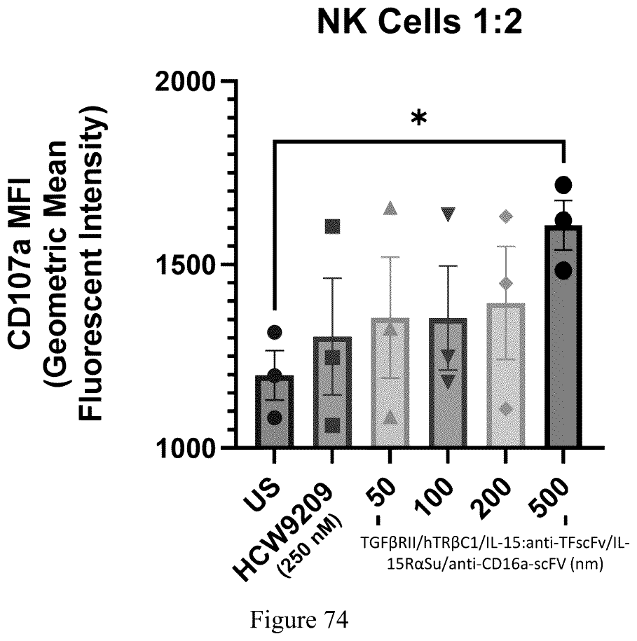

Frozen human peripheral blood mononuclear cells (PBMCs) were mixed with A431 in (2:1 E:T) ratio. At the same time, CD107a was added in culture. After 1 hr BFA/Monensin was added and further cultured for 4 hours. Cells were harvested and stained for CD3, CD16, CD56, and CD107a for FACS analysis. NB: Cells were gated on CD3⁺ CD16⁺ CD56⁺ cells. See FIG. 74. The results show TGFβRII/hTRβC1/IL-15:anti-TFscFv/IL-15RαSu/anti-CD16a-scFv induction of CD107a in human PBMCs.

### Example 44: IL-7/hTRβC1/IL-15:IL-21/IL-15RαSu Protein Complex

The nucleic acid sequence of the IL-21/IL-15RαSu construct is as follows (SEQ ID NO: 197):

(Signal peptide):
ATGGGAGTGAAAGTTCTTTTTGCCCTTATTTGTATTGCTGTGGCCGAGGCC
(IL-21):
CAAGGTCAAGATCGCCACATGATTAGAATGCGTCAACTTATAGATATTGTTGATCAGCTGAAAA
ATTATGTGAATGACTTGGTCCCTGAATTTCTGCCAGCTCCAGAAGATGTAGAGACAAACTGTGA
GTGGTCAGCTTTTTCCTGTTTTCAGAAGGCCCAACTAAAGTCAGCAAATACAGGAAACAATGAA
AGGATAATCAATGTATCAATTAAAAAGCTGAAGAGGAAACCACCTTCCACAAATGCAGGGAGAA
GACAGAAACACAGACTAACATGCCCTTCATGTGATTCTTATGAGAAAAAACCACCCAAAGAATT
CCTAGAAAGATTCAAATCACTTCTCCAAAAGATGATTCATCAGCATCTGTCCTCTAGAACACAC
GGAAGTGAAGATTCC
(Human IL-15Rα sushi domain):
ATCACGTGCCCTCCCCCCATGTCCGTGGAACACGCAGACATCTGGGTCAAGAGCTACAGCTTGT
ACTCCAGGGAGCGGTACATTTGTAACTCTGGTTTCAAGCGTAAAGCCGGCACGTCCAGCCTGAC
GGAGTGCGTGTTGAACAAGGCCACGAATGTCGCCCACTGGACAACCCCCAGTCTCAAATGCATT
AGATGATAACGCGTACGAAG.

The amino acid sequence of the IL-21/IL-15RαSu fusion protein is as follows (SEQ ID NO: 147):

QGQDRHMIRMRQLIDIVDQLKNYVNDLVPEFLPAPEDVETNCEWSAFSC
FQKAQLKSANTGNNERIINVSIKKLKRKPPSTNAGRRQKHRLTCPSCDS
YEKKPPKEFLERFKSLLQKMIHQHLSSRTHGSEDSITCPPPMSVEHADI
WVKSYSLYSRERYICNSGFKRKAGTSSLTECVLNKATNVAHWTTPSLKC
IR.

The nucleic acid sequence of the IL-7/hTRβC1/IL-15 construct is as follows (SEQ ID NO: 1XX):

(Signal peptide):
ATGGGAGTGAAAGTTCTTTTTGCCCTTATTTGTATTGCTGTGGCCGAGGCC
(Human IL-7):
GATTGTGATATTGAAGGTAAAGATGGCAAACAATATGAGAGTGTTCTAATGGTCAGCATCGATC
AATTATTGGACAGCATGAAAGAAATTGGTAGCAATTGCCTGAATAATGAATTTAACTTTTTTAA
AAGACATATCTGTGATGCTAATAAGGAAGGTATGTTTTTATTCCGTGCTGCTCGCAAGTTGAGG
CAATTTCTTAAAATGAATAGCACTGGTGATTTTGATCTCCACTTATTAAAAGTTTCAGAAGGCA
CAACAATACTGTTGAACTGCACTGGCCAGGTTAAAGGAAGAAAACCAGCTGCCCTGGGTGAAGC
CCAACCAACAAAGAGTTTGGAAGAAAATAAATCTTTAAAGGAACAGAAAAAACTGAATGACTTG
TGTTTCCTAAAGAGACTATTACAAGAGATAAAAACTTGTTGGAATAAAATTTTGATGGGCACTA
AAGAACAC
(Human TRBC1):
GAGGACCTGAACAAGGTGTTCCCACCCGAGGTCGCTGTGTTTGAGCCATCAGAAGCAGAGATCT
CCCACACCCAAAAGGCCACACTGGTGTGCCTGGCCACAGGCTTCTTCCCCGACCACGTGGAGCT
GAGCTGGTGGGTGAATGGGAAGGAGGTGCACAGTGGGGTCAGCACGGACCCGCAGCCCCTCAAG
GAGCAGCCCGCCCTCAATGACTCCAGATACTGCCTGAGCAGCCGCCTGAGGGTCTCGGCCACCT
TCTGGCAGAACCCCCGCAACCACTTCCGCTGTCAAGTCCAGTTCTACGGGCTCTCGGAGAATGA
CGAGTGGACCCAGGATAGGGCCAAACCCGTCACCCAGATCGTCAGCGCCGAGGCCTGGGGTAGA
GCAGAC
(Human IL-15):
AACTGGGTGAATGTAATAAGTGATTTGAAAAAAATTGAAGATCTTATTCAATCTATGCATATTG
ATGCTACTTTATATACGGAAAGTGATGTTCACCCCAGTTGCAAAGTAACAGCAATGAAGTGCTT
TCTCTTGGAGTTACAAGTTATTTCACTTGAGTCCGGAGATGCAAGTATTCATGATACAGTAGAA
AATCTGATCATCCTAGCAAACAACAGTTTGTCTTCTAATGGGAATGTAACAGAATCTGGATGCA
AAGAATGTGAGGAACTGGAGGAAAAAAATATTAAAGAATTTTTGCAGAGTTTTGTACATATTGT
CCAAATGTTCATCAACACTTCTTGATAACGCGTACGAAG.

The amino acid sequence of the IL-7/hTRβC1/IL-15 fusion protein is as follows (SEQ ID NO: 140):

DCDIEGKDGKQYESVLMVSIDQLLDSMKEIGSNCLNNEFNFFKRHICDA
NKEGMFLFRAARKLRQFLKMNSTGDFDLHLLKVSEGTTILLNCTGQVKG
RKPAALGEAQPTKSLEENKSLKEQKKLNDLCFLKRLLQEIKTCWNKILM
GTKEHEDLNKVFPPEVAVFEPSEAEISHTQKATLVCLATGFFPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADNWVNVISDLKKI
EDLIQSMHIDATLYTESDVHPSCKVTAMKCFLLELQVISLESGDASIHD
TVENLIILANNSLSSNGNVTESGCKECEELEEKNIKEFLQSFVHIVQMF
INTS.

### Example 45: In Vitro Stimulation of Raji-PDL1/hPBMC Organoids Using IL-7/hTRpC1/IL-15: IL-21/IL-15RαSu and TGFβRII/hTRβC1/IL-15: Anti-PD-1(Diabody)/IL-15RαSu/IL-7

The in vitro effects of multi-chain chimeric polypeptides were evaluated in 3-D organoid cultures containing tumor cells and human immune cells. Organoids composed of 10:1 E:T of hPBMCs:Raji-PDL1 were made. The organoids were resuspended in 1:2 Matrigel. Four domes were made at 20 uL/dome in each well of a 12 well plate. See Table 1.

TABLE 1

| Dome: 20 uL | hPBMC: 1 × 10⁵/dome | |
|---|---|---|
| | Raji-PDL1 cells: 1 × 10⁴/dome | |
| hPBMC/Raji-w/o | 1 | |
| stimuli | | |
| hPBMC/Raji- | 2: IL-7/hTRβC1/IL- | 3: TGFβRII/hTRβC1/IL-15: |
| w/stimuli | 15: IL-21/IL-15RαSu | anti-PD-1(diabody)/IL- |
| | | 15RαSu/IL-7 |

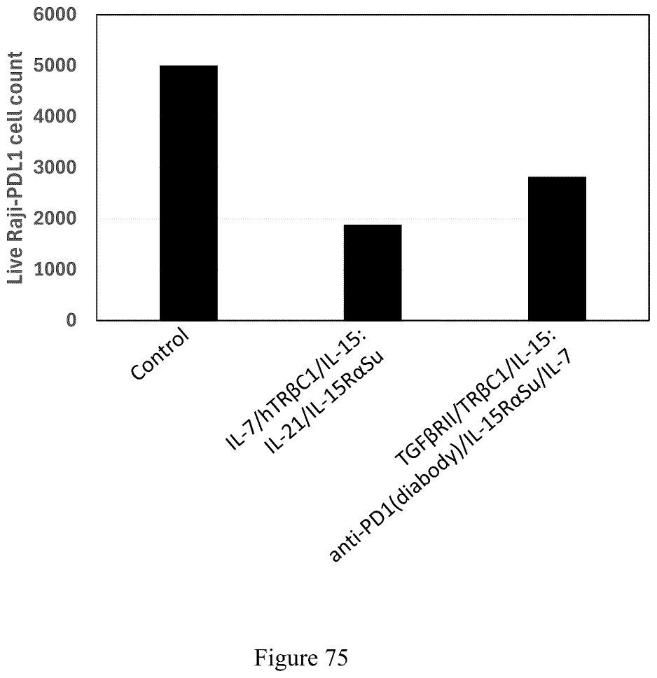

After 5 days in culture, the organoids were dissolved, harvested, and stained for CD8, CD4, PDL-1, and PI-Live/Dead. Data shown: results from 20 sec flow collected ˜20 μl/600 μl total. See FIG. 75.

The results show that both IL-7/hTRβC1/IL-15: IL-21/IL-15RαSu and TGFβRII/hTRβC1/IL-15: anti-PD-1(diabody)/IL-15RαSu/IL-7 significantly increase cytotoxicity of human PBMCs.

### Example 46: Cytotoxicity of Human PBMCs Against Raji Cells in Matrigel Activated by HCW Molecules

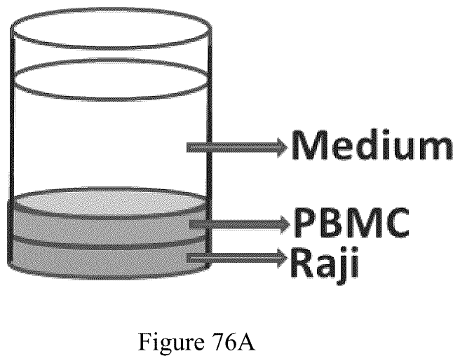

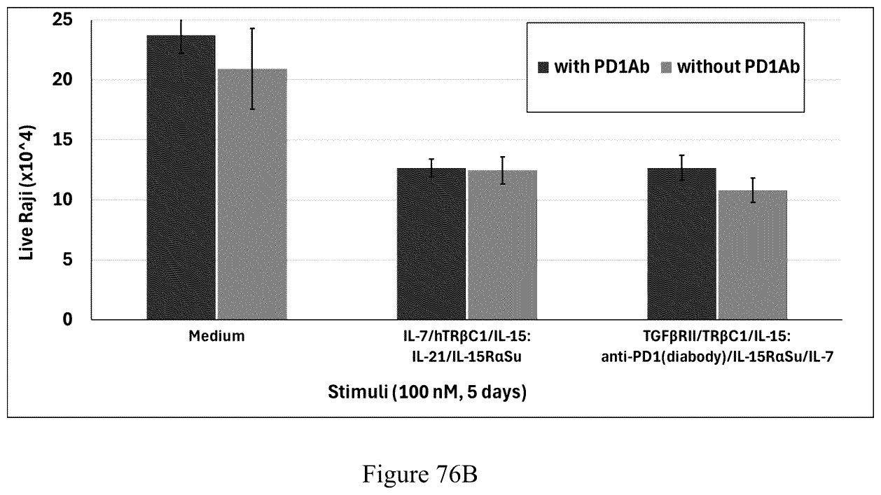

Two layer cultures were established using 2×10⁴ Raji-PDL1 cells (100 μL matrigel) overlayered with 4×10⁵ frozen human PBMCs (100 μL matrigel), in wells in 48-well plate. See FIG. 76A. RPMI-10 (800 μL/well) with IL-7/hTRβC1/IL-15: IL-21/IL-15RαSu or TGFβRII/hTRβC1/IL-15: anti-PD-1(diabody)/IL-15RαSu/IL-7 each at 100 nM was added. The cultures were harvested 5 days later. The live Raji-PDL1 cells were counted by flow cytometer. See FIG. 76B.

The results show that both IL-7/hTRβC1/IL-15: IL-21/IL-15RαSu and TGFβRII/hTRβC1/IL-15: anti-PD-1(diabody)/IL-15RαSu/IL-7 significantly increase cytotoxicity of human PBMCs. However anti-human PD-1 antibody does not further enhance the cytotoxicity of PBMCs.

### Example 47: In Vitro Stimulation of hPBMCs Cocultured with Raji-PDL1 Organoids Using IL-7/hTRβC1/IL-15: IL-21/IL-15RαSu and TGFβRII/hTRβC1/IL-15: Anti-PD-1(Diabody)/IL-15RαSu/IL-7

Organoids composed of 9×10⁴ Raji-PDL1 cells per dome were made at two different Matrigel (MG) concentrations (1:1 and 1:2). There were 3 domes/well. After culture for 3 days, active PBMCs, native PBMCs, and/or HCW molecules were added to the well and the plate was incubated at 37° C. for culture. See Table 2.

TABLE 2

| | | |
|---|---|---|
| Dome: 20 uL | 9000 Raji-PDL1 cells/dome | 9000 Raji-PDL1 cells/dome |
| | (1:1 MG) | (1:2 MG) |
| Tumor Only/No Stimuli | 1 | 4 |
| hPMBCs in well with | 2: ahPMBCs 1.5 × 10⁶ and | 5: nhPMBCs 4 × 10⁶ and |
| IL-7/hTRβC1/IL-15: | 100 nM IL-7/hTRβC1/IL-15: | 100 nM IL-7/hTRβC1/IL-15: |
| IL-21/IL-15RαSu | IL-21/IL-15RαSu | IL-21/IL-15RαSu |
| hPMBCs in well with | 3: ahPMBCs 1.5 × 10⁶ and | 6: nhPMBCs 4 × 10⁶ and |
| TGFβRII/hTRβC1/IL-15: | 100 nM TGFβRII/hTRβC1/ | 100 nM TGFβRII/hTRβC1/IL-15: |
| anti-PD-1(diabody)/ | IL-15: anti-PD-1(diabody)/ | anti-PD-1(diabody)/ |
| IL-15RαSu/IL-7 | IL-15RαSu/IL-7 | IL-15RαSu/IL-7 |

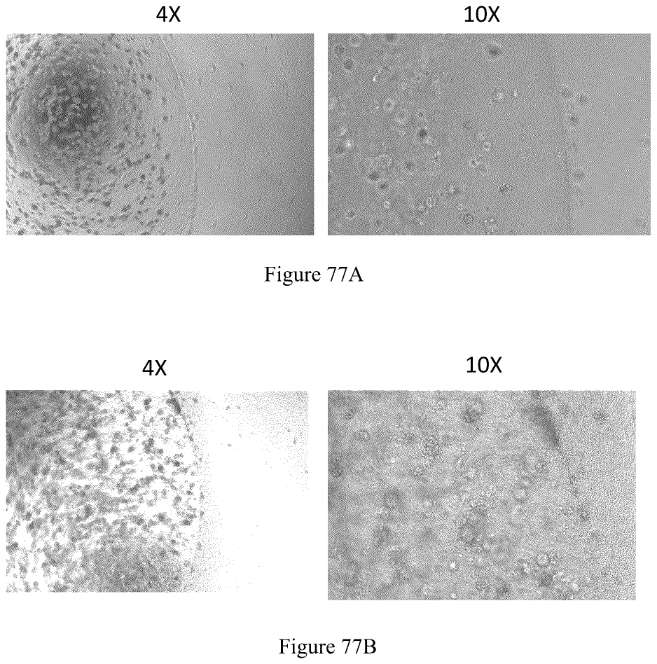

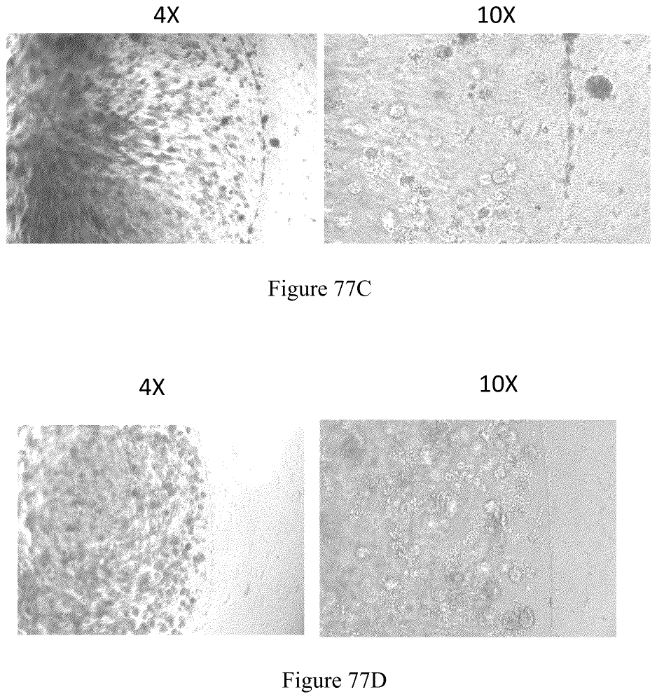

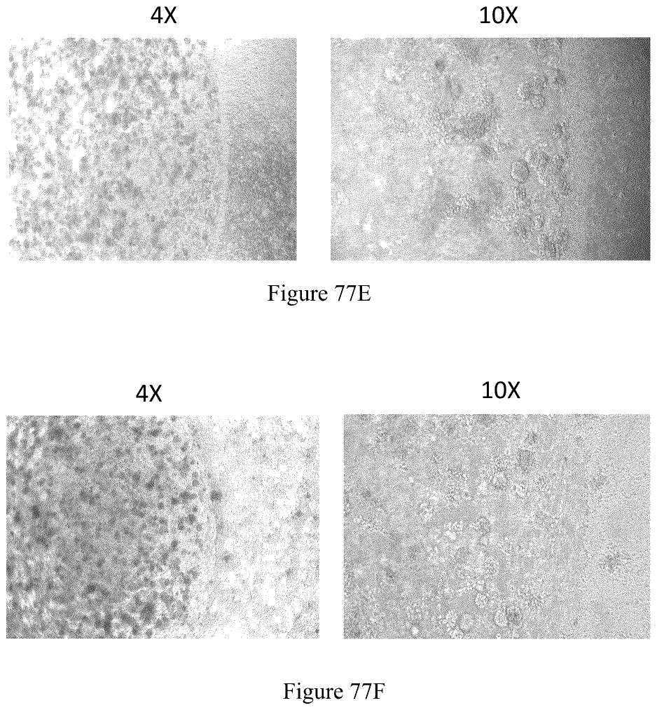

Two days post-treatment, images were taken of the individual dome colonies. See FIGS. 77A-77F. The results showed that IL-7/hTRβC1/IL-15: IL-21/IL-15RαSu- and TGFβRII/hTRβC1/IL-15: anti-PD-1(diabody)/IL-15RαSu/IL-7-activated PBMCs infiltrated into the Raji-PDL1 matrigel organoids.

### Example 48: Apoptosis Assay for TGFβRII/hTRβC1/IL-15: Anti-TFscFv/IL-15RαSu/Anti-CD3scFv in ASPC1 Spheroid

Cell Culture. ASPC1 (human pancreatic adenocarcinoma) cells were cultured in RPMI-1640 medium supplemented with 10% fetal bovine serum (FBS) and 1% penicillin/streptomycin (P/S), referred to as RIO medium. Cells were maintained at 37° C. in a humidified incubator with 5% CO₂ and 95% air.

Cell Harvesting. When cells reached 70-80% confluency, the old medium was removed and discarded. The cell monolayer was briefly rinsed with DPBS, followed by the addition of trypsin or trypsin/EDTA solution to detach the cells. Trypsin was neutralized by adding fresh RIO medium.

Spheroid Preparation. Cells were counted, and 25,000 cells were resuspended in 10 μL of Matrigel Matrix. The mixture was seeded into black 96-well polystyrene microplates. Plates were incubated for 30 minutes at 37° C. in a humidified atmosphere of 5% CO₂ and 95% air to allow spheroid formation.

Culture Maintenance. After incubation, 200 μL of warm R10 medium was gently added to each well. The spheroids were cultured for 72 hours under the same incubation conditions.

PBMC Preparation. Peripheral blood mononuclear cells (PBMCs) from three donors were thawed and counted.

Assay. The culture medium was gently removed from each well, and 100 μL of fresh, warm RIO medium was added with PBMC in E:T in 1:1, 5:1 and 10:1 with TGFβRII/hTRβC1/IL-15: anti-TFscFv/IL-15RαSu/anti-CD3scFv 20 nM in final concentration. Controls wells were also setup for each condition media alone, ASPC-1 cell alone and PBMC alone.

Caspase-Glo® 3/7 3D reagent was prepared according to the manufacturer's instructions. Then, 100 μL of the reagent was added to each well. The plate contents were mixed on a shaker at 500 rpm for 30 seconds and then incubated at room temperature for 30 minutes. Luminescence was measured using a plate-reading luminometer.

Data Analysis. Luminescence values for each sample were recorded and plotted for analysis.

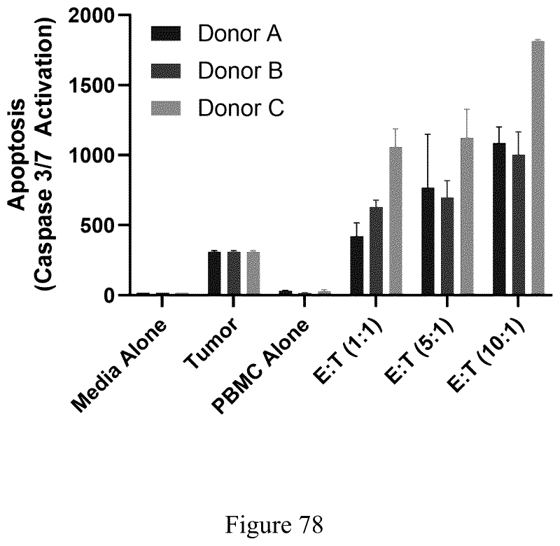

Results. Apoptosis activity in the spheroid tumor cell culture increased with increasing E:T ratios for BPMCS of all 3 donors. See FIG. 78.

### Example 49: Immunohistochemistry of ASPC1 Tumor after Treatment with HCW11-18b

Methods. A suspension of ASPC1 cells was prepared in a 1:1 ratio of PBS and Matrigel. Each NSG mouse was subcutaneously injected in the right flank with 2×10⁶ ASPC1 cells in a total volume of 100 μL.

Tumors were allowed to grow for 8 days prior to treatment. On Day 8 post-tumor inoculation, 20×10⁶ purified human T cells were intravenously infused into each mouse via tail vein injection. Mice were treated with 1 mg/kg of TGFβRII/hTRβC1/IL-15: anti-TFscFv/IL-15RαSu/anti-CD3scFv via subcutaneous injection every 5 days, for a total of 6 doses administered on Days 0, 5, 10, 15, 20, and 25. On Day 28 (three days after the final dose), mice were euthanized.

Tumors were excised and fixed in Formalin for 24-48 hours. Fixed tissues were then transferred to ethanol and processed for paraffin embedding. Formalin-Fixed Paraffin-Embedded (FFPE) tissue blocks were prepared. Sections of 4-5 μm thickness were cut from the FFPE blocks.

Immunohistochemistry (IHC) staining was performed using validated protocols with the following primary antibodies: Anti-CD3 (to assess T cell infiltration) and Anti-Tissue Factor (to assess TF expression) separately in different slides. Stained slides were examined under a bright-field microscope, representative images were captured at consistent magnifications across all samples and subsequently analyzed using ImageJ software.

CD3⁺ and TF⁺ cells were counted across multiple high-power fields (HPFs). The average number of positive cells per HPF was calculated for each sample. For each sample, an H-score was calculated. CD3 and Tissue Factor expression levels were compared between treatment groups. Statistical analysis (unpaired t-test) was performed.

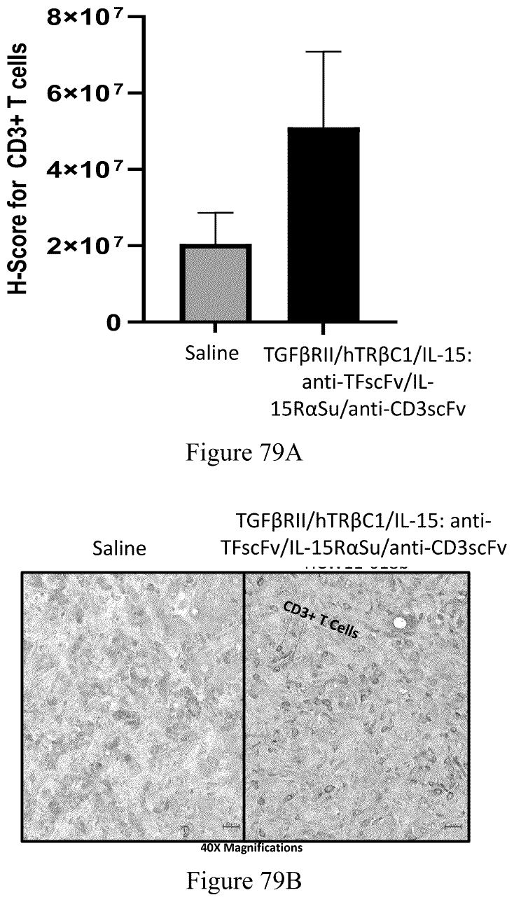

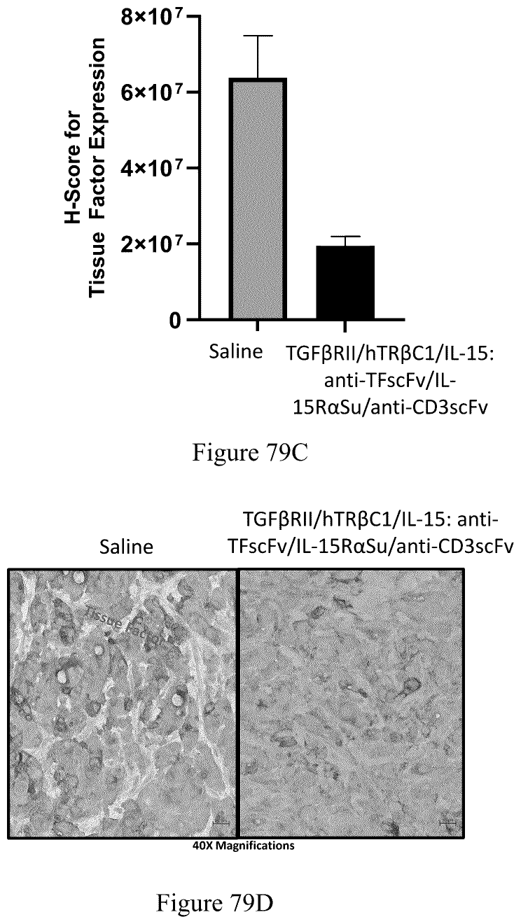

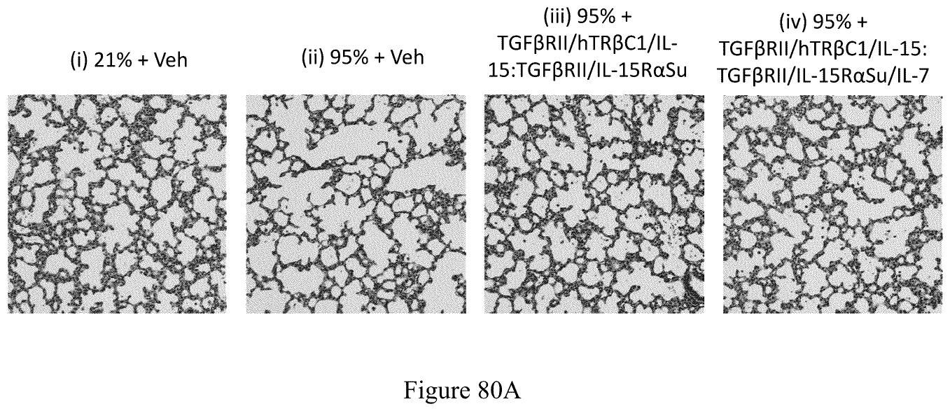

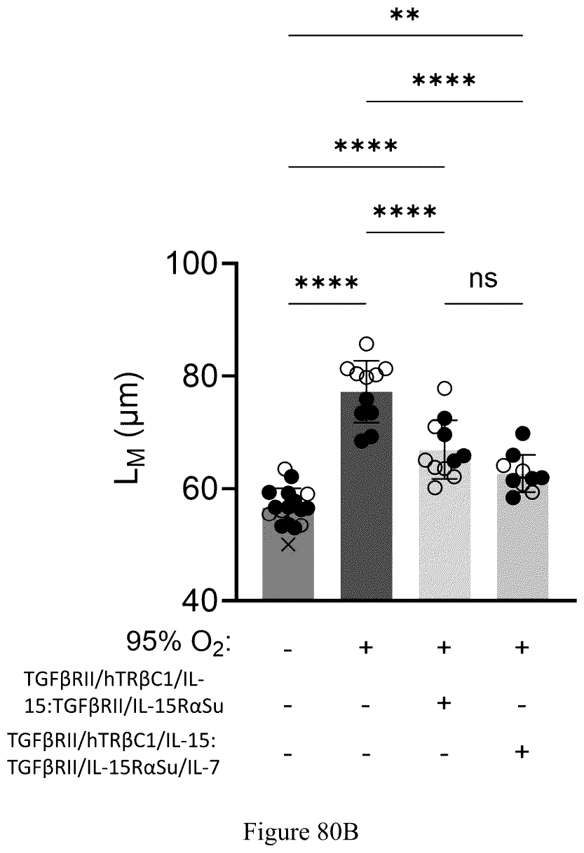

Results. The results show that treatment with TGFβRII/hTRβC1/IL-15: anti-TFscFv/IL-15RαSu/anti-CD3scFv induced human T cell infiltration into ASPC1 tumors and TF expression decreased in the tumor sections. See FIGS. 79A-79D.

### Example 50: TGFβRII/hTRβC1/IL-15:TGFβRII/IL-15RαSu or TGFβRII/hTRβC1/IL-15: TGFβRIIL-15RαSu/IL-7 Reduce Hyperoxia-Induced Broncho Pulmonary Dysplasia (BPD) Models

Methods. Mouse model of bronchopulmonary dysplasia and quantification of Lung morphometry: Pups from timed-pregnant dams (Charles River) were randomized at birth (P0) into four groups: (i) Normoxia+Vehicle, (ii) Hyperoxia+Vehicle, (iii) Hyperoxia+TGFβRII/hTRβC1/IL-15:TGFβRII/IL-15RαSu, or (iv) Hyperoxia+TGFβRII/hTRβC1/IL-15: TGFβRII/IL-15RαSu/IL-7. Groups i and ii received 5 μL subcutaneous (S.Q.) injections of sterile PBS, while groups (iii) and (iv) received 5 μL S.Q. injections of TGFβRII/hTRβC1/IL-15:TGFβRII/IL-15RαSu or TGFβRII/hTRβC1/IL-15: TGFβRII/IL-15RαSu/IL-7 (10 mg/kg in PBS). Pups and dams were exposed to either 21% (normoxia) or 95% (hyperoxia) oxygen from P0 to P3, with dams rotated between chambers every 24 hours to maintain maternal health. On P7, pups were euthanized; lungs were flushed with PBS/heparin via the right ventricle and inflated with 2% PFA, then fixed for 48 hours. Lungs were embedded in OCT for arteriolar density analysis or in paraffin for morphometric assessment. Mean linear intercept (LM) scores were calculated in FIJI by overlaying a line of known length on distal lung regions, avoiding bronchioles and vessels, and counting alveolar wall intersections.

Results. Both TGFβRII/hTRβC1/IL-15:TGFβRII/IL-15RαSu and TGFβRII/hTRβC1/IL-15: TGFβRII/IL-15RαSu/IL-7 significantly improved airway structure, as evidenced by reduced mean linear intercept (MLI) compared to hyperoxia controls. TGFβRII/hTRβC1/IL-15: TGFβRII/IL-15RαSu/IL-7 demonstrated a greater effect, with lower variability and MLI values approaching normoxic baseline levels.

1. A multi-chain chimeric polypeptide comprising:
(a) a first chimeric polypeptide comprising:
(i) a first target-binding domain;
(ii) a soluble T cell receptor β-chain constant region (TRβC) domain; and
(iii) a first domain of a pair of affinity domains; and
(b) a second chimeric polypeptide comprising:
(i) a second domain of a pair of affinity domains; and
(ii) a second target-binding domain,
wherein the first chimeric polypeptide and the second chimeric polypeptide associate through the binding of the first domain and the second domain of the pair of affinity domains.

2. The multi-chain chimeric polypeptide of claim 1, wherein the first target-binding domain and the soluble TRβC domain directly abut each other in the first chimeric polypeptide.

3. The multi-chain chimeric polypeptide of claim 1, wherein the first chimeric polypeptide further comprises a linker sequence between the first target-binding domain and the soluble TRβC domain in the first chimeric polypeptide.

4. The multi-chain chimeric polypeptide of claim 1, wherein the soluble TRβC domain and the first domain of the pair of affinity domains directly abut each other in the first chimeric polypeptide.

5. The multi-chain chimeric polypeptide of claim 1, wherein the first chimeric polypeptide further comprises a linker sequence between the soluble TRβC domain and the first domain of the pair of affinity domains in the first chimeric polypeptide.

6. The multi-chain chimeric polypeptide of claim 1, wherein the second domain of the pair of affinity domains and the second target-binding domain directly abut each other in the second chimeric polypeptide.

7. The multi-chain chimeric polypeptide of claim 1, wherein the second chimeric polypeptide further comprises a linker sequence between the second domain of the pair of affinity domains and the second target-binding domain in the second chimeric polypeptide.

8. The multi-chain chimeric polypeptide of claim 1, wherein the first chimeric polypeptide and/or the second chimeric polypeptide further comprises one or more additional target-binding domains.

9.-14. (canceled)

15. The multi-chain chimeric polypeptide of claim 8, wherein the one or more additional target-binding domains comprises an anti-PD-1 diabody or scFv, e.g., one derived from pembrolizumab.

16.-17. (canceled)

18. The multi-chain chimeric polypeptide of claim 1, wherein one or both of the first target-binding domain and the second target-binding domain is an antigen-binding domain.

19. The multi-chain chimeric polypeptide of claim 1, wherein one or both of the first target-binding domain and the second target-binding domain bind specifically to a target selected from the group consisting of: Tissue Factor (CD142), CD16a, CD28, CD3, CD33, CD20, CD19, CD22, CD123, IL-1R, IL-1, VEGF, IL-6R, IL-4, IL-10, PDL-1, TIGIT, PD-1, TIM3, CTLA4, MICA, MICB, IL-6, IL-8, TNFα, CD26a, CD36, ULBP2, CD30, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5/6, uPAR, a UL16-binding protein, HLA-DR, DLL4, TYRO3, AXL, MER, CD122, CD155, PDGFDD, a ligand of TGF-β receptor II (TGF-βRII), a ligand of TGF-βRIII, a ligand of DNAM1, a ligand of NKp46, a ligand of NKp44, a ligand of NKG2D, a ligand of NKp30, a ligand for a scMHCI, a ligand for a scMHCII, a ligand of ICOS, a receptor for IL-1, a receptor for IL-2, a receptor for IL-3, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-DD, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28.

20. The multi-chain chimeric polypeptide of claim 1, wherein one or both of the first target-binding domain and the second target-binding domain is a soluble interleukin or cytokine protein.

21. The multi-chain chimeric polypeptide of claim 20, wherein the soluble interleukin, cytokine, or ligand protein is selected from the group consisting of: IL-1, IL-2, IL-3, IL-4, IL-7, IL-8, IL-10, IL-12, IL-15, IL-17, IL-18, IL-21, PDGF-DD, SCF, and FLT3L.

22. (canceled)

23. The multi-chain chimeric polypeptide of claim 1, wherein one or both of the first target-binding domain and the second target-binding domain is a soluble receptor.

24. The multi-chain chimeric polypeptide of claim 23, wherein the soluble receptor is a soluble TGF-β receptor II (TGF-βRII), a soluble TGF-βRIII, a soluble NKG2D, a soluble NKp30, a soluble NKp44, a soluble NKp46, a soluble DNAM-1, a scMHCI, a scMHCII, a soluble CD155, or a soluble CD28.

25. (canceled)

26. The multi-chain chimeric polypeptide of claim 1, wherein:
the first target-binding domain is IL-7 and the second target-binding domain is a soluble TGF-βRII, optionally, wherein the second chimeric polypeptide further comprises an anti-PD-1 antigen-binding domain or an anti-tissue factor antigen-binding domain;
the first target-binding domain is a soluble TGF-βRII and the second target-binding domain is a soluble TGF-βRII;
the first target-binding domain is a soluble IL-7 and the second target-binding domain is a soluble IL-21;
the first target-binding domain is soluble IL-7 and the second target-binding domain is an anti-CD3 antigen-binding domain, optionally, wherein the second chimeric polypeptide further comprises an anti-tissue factor antigen-binding domain;
the first target-binding domain is a soluble IL-7 and the second target-binding domain is an anti-PD-1 antigen-binding domain, optionally, wherein the second chimeric polypeptide further comprises a soluble IL-21;
the first target-binding domain is a soluble IL-7 and the second antigen-binding domain is an anti-tissue factor antigen-binding domain, optionally, wherein the second chimeric polypeptide further comprises an anti-CD3 antigen-binding domain;
the first target-binding domain is a soluble TGF-βRII and the second target-binding domain is a soluble anti-tissue factor antigen-binding domain, optionally, wherein the second chimeric polypeptide further comprises an anti-CD16 antigen-binding domain or an anti-CD3 antigen-binding domain;
the first target-binding domain is a soluble TGF-βRII and the second target-binding domain is an anti-PD-1 antigen-binding domain, optionally, wherein the second chimeric polypeptide further comprises a soluble IL-7:
the first target-binding domain is a soluble TGF-βRII and the second target-binding domain is an anti-CD3 antigen-binding domain, optionally, wherein the second chimeric polypeptide further comprises an anti-tissue factor antigen-binding domain; or
the first target-binding domain is an anti-tissue factor antigen-binding domain and the second target-binding domain is an anti-PD-1 antigen-binding domain, optionally, wherein the second chimeric polypeptide further comprises a soluble IL-7.

27.-51. (canceled)

52. The multi-chain chimeric polypeptide of claim 1, wherein the soluble TRβC domain is a soluble human TRβC domain.

53. The multi-chain chimeric polypeptide of claim 1, wherein the soluble TRβC domain does not bind to an MHC protein with or without a loaded antigen and/or the soluble TRβC domain does not stimulate an immune response in a mammal.

54.-57. (canceled)

58. The multi-chain chimeric polypeptide of claim 1, wherein the pair of affinity domains is a sushi domain from an alpha chain of human IL-15 receptor (IL-15Rα) and a soluble IL-15.

59. The multi-chain chimeric polypeptide of claim 1, wherein the pair of affinity domains is selected from the group consisting of: barnase and barnstar, a PKA and an AKAP, adapter/docking tag modules based on mutated RNase I fragments, and SNARE modules based on interactions of the proteins syntaxin, synaptotagmin, synaptobrevin, and SNAP25.

60. (canceled)

61. A composition comprising the multi-chain chimeric polypeptide of claim 1.

62. (canceled)

63. A kit comprising at least one dose of the composition of claim 61.

64. A method of stimulating an immune cell, the method comprising:
contacting an immune cell with an effective amount of the multi-chain chimeric polypeptide of claim 1.

65. A method of inducing or increasing proliferation of an immune cell, the method comprising:
contacting an immune cell with an effective amount of the multi-chain chimeric polypeptide of claim 1.

66. A method of inducing differentiation of an immune cell into a memory or memory-like immune cell, the method comprising:
contacting an immune cell with an effective amount of the multi-chain chimeric polypeptide of claim 1.

67.-70. (canceled)

71. A method of killing a cancer cell, an infected cell, or a senescent cell in a subject in need thereof, the method comprising administering to the subject a therapeutically effective amount of the multi-chain chimeric polypeptide of claim 1.

72. A method of treating a subject in need thereof, the method comprising administering to the subject a therapeutically effective amount of the multi-chain chimeric polypeptide of claim 1.

73.-76. (canceled)

77. Nucleic acid encoding the multi-chain chimeric polypeptide of claim 1.

78. (canceled)

79. A cell comprising the nucleic acid of claim 77.

80. A method of producing a multi-chain chimeric polypeptide, the method comprising:
culturing the cell of claim 79 in a culture medium under conditions sufficient to result in the production of the multi-chain chimeric polypeptide; and
recovering the multi-chain chimeric polypeptide from the cell and/or the culture medium.

81. (canceled)


SEQUENCE LISTING (198 sequences)

SEQ ID NO: 1 - protein, 130 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 2 - DNA, 390 bp, Homo sapiens
  source 1..390: mol_type = genomic DNA
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagac

SEQ ID NO: 3 - protein, 130 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 4 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 5 - protein, 133 aa, Homo sapiens [sequence omitted]

SEQ ID NO: 6 - DNA, 399 bp, Homo sapiens
  source 1..399: mol_type = genomic DNA
caaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 7 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - DNA, 408 bp, synthetic construct
  source 1..408: mol_type = other DNA
atcccgccacacgtgcaaaagagcgtgaacaacgacatgatcgttacggacaacaatggggccgtgaagttccctcagttatgcaaattctgtgacgtgagattcagcacgtgcgacaatcagaagagctgcatgtccaattgctcaatcacaagcatctgcgagaaaccccaagaagtttgcgtggccgtgtggagaaagaatgacgagaacatcaccctggaaaccgtgtgccatgatcctaaactcccttaccatgactttatcttggaggatgctgctagcccaaagtgcatcatgaaagagaagaagaaaccgggggaaacttttttcatgtgttcatgcagctctgacgagtgtaatgataacattatcttcagcgaagagtataacactagcaatcctgat

SEQ ID NO: 9 - protein, 277 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 10 - DNA, 831 bp, synthetic construct
  source 1..831: mol_type = other DNA
atcccgccacacgtgcaaaagagcgtgaacaacgacatgatcgttacggacaacaatggggccgtgaagttccctcagttatgcaaattctgtgacgtgagattcagcacgtgcgacaatcagaagagctgcatgtccaattgctcaatcacaagcatctgcgagaaaccccaagaagtttgcgtggccgtgtggagaaagaatgacgagaacatcaccctggaaaccgtgtgccatgatcctaaactcccttaccatgactttatcttggaggatgctgctagcccaaagtgcatcatgaaagagaagaagaaaccgggggaaacttttttcatgtgttcatgcagctctgacgagtgtaatgataacattatcttcagcgaagagtataacactagcaatcctgatggaggtggcggatctatccccccccatgtgcaaaaatcagtgaacaatgacatgatcgtgaccgataataacggggcggtaaagtttcctcaactgtgtaaattttgcgacgtcagattcagcacatgcgacaatcaaaagtcctgcatgagcaactgcagcatcacctctatctgtgagaaaccacaagaagtatgtgtggcggtatggcggaagaacgatgaaaatatcacgctggagaccgtttgccatgatcctaagctgccatatcacgatttcatcctggaggacgccgctagccctaagtgtatcatgaaggagaagaagaagcccggtgagacctttttcatgtgctcatgcagctccgacgagtgcaacgacaacatcatcttcagcgaggagtacaatacaagcaatcctgac

SEQ ID NO: 11 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 12 - DNA, 196 bp, synthetic construct
  source 1..196: mol_type = other DNA
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagag

SEQ ID NO: 13 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 14 - DNA, 342 bp, synthetic construct
  source 1..342: mol_type = other DNA
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 15 - protein, 473 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 16 - DNA, 1419 bp, synthetic construct
  source 1..1419: mol_type = other DNA
gatattcagatgacacagtcacctgcctcactgtcagcgtctgtgggggaccgcgtgacaattacgtgcctggctagccaaaccatagatacctggctggcgtggtacttgcagaaacctggaaagtcgccacagctgctgatctatgcggcaactaacctggcggacggcgtaccctcgcgtttttccgggtctggcagcggtaccgatttctccttcactattagcagccttcagccggaggactttgctacatattattgtcagcaagtgtactcatcccctttcaccttcggccaaggcaccaaactagaaataaaaggcgggggcggttcacaaattcagctggtgcaaagcggcggcgaagtgaagaagcctggagctagcgtgcgtgtgagctgtaaggcatctggctactccttcactgattacaacgtgtattgggttagacagtcccctggtaaagggcttgagtggattggctatatagacccttataacggcattaccatatacgatcaaaacttcaaaggtaaggccaccctgaccgttgacaagtcaacaagcaccgcctacatggagctgagcagcctaagaagcgaggacaccgctgtctatttctgtgctagagacgtaaccacagccctggatttctggggccaaggcacaacggtgacagtcagctcgggtggcggcggttcgggtgggggaggaagcgggggggggggatccgacattcaaatgacgcaaagccctgctagcctttccgctagcgtgggagaccgcgttacgatcacatgtctggcctctcagaccatcgacacttggctggcctggtacctacaaaaacctggcaaaagcccacaactactgatctatgctgctacgaatctggccgacggcgtaccgtcccgcttcagcggctccggttctgggacagacttctctttcaccatttcatctctgcagcctgaagatttcgccacatattattgtcaacaagtgtatagcagcccgtttacctttgggcaaggcacgaagttagaaatcaagggcggaggcggatcccaaattcagctcgtgcagagtggcggggaagtcaagaaacctggcgctagcgtgagagtgtcttgtaaggcaagtggctacagctttaccgactataatgtgtattgggtgagacaatcacccggcaaggggctggagtggatcggctacatcgacccgtataacggcattacgatctacgaccaaaactttaaaggcaaggccaccctaacggtcgacaagtcgacctccaccgcttatatggagcttagctcattgagaagcgaggataccgcggtgtacttctgcgctagagacgtgacaaccgccctggacttctgggggcaaggcacaaccgtcaccgtgtcgtcc

SEQ ID NO: 17 - protein, 244 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 18 - DNA, 732 bp, synthetic construct
  source 1..732: mol_type = other DNA
gatattcagatgacgcagtcgccaagctcactctcggcctccgtaggagacagagttaccatcacctgcagagctagccaagacatcagaaactacctgaactggtatcagcagaagcctggcaaggcccctaagctcctgatctactacacgagcagacttgagagcggggtaccatcccgcttcagcggatctggaagcggcacagactacaccctgaccatctctagtctgcagcctgaggacttcgccacttactattgtcagcaaggcaacaccctgccttggacctttggccaaggcaccaaagttgagataaagggagggggagggagcggtggcggaggctcgggtgggggtggctcagaagtacagctggttgagtccggcggtggtctggtgcagcctggcggaagcctgagactgagctgcgccgcaagcggctatagcttcactggctataccatgaactgggtgcgccaagcccctggcaagggcctggaatgggtggccctgatcaacccttacaagggcgtgagcacctacaatcagaagttcaaggacagattcaccatcagcgtggacaagagcaagaacaccgcctacctgcagatgaacagcctgagagccgaggacactgcggtctattactgtgctagaagcggctactacggcgacagcgactggtacttcgacgtgtgggggcaaggcaccttggtaacggtaagctcc

SEQ ID NO: 19 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 20 - DNA, 729 bp, synthetic construct
  source 1..729: mol_type = other DNA
gacatcaagctgcagcagagcggcgccgagctggctagacctggggctagcgtcaagatgagctgcaagacaagcggctacactttcacaagatacaccatgcactgggtgaagcagagacctggccaaggcctggaatggatcggttatatcaaccctagcagaggctacaccaactacaatcagaagttcaaggacaaagccaccctgacaactgataagagcagcagcaccgcctatatgcaactgtcaagcctcacctcagaggacagcgccgtgtactactgcgctagatactacgacgaccactactgcctggactattggggccaaggcactactttgactgttagctcggtcgagggaggctccggcgggagtggtggttctggcggcagcggcggagttgacgacattcagctgacacaaagtcctgccataatgagcgctagccctggcgagaaggtgaccatgacctgcagagctagcagcagcgtgagctacatgaactggtatcagcagaagagcggcacaagccctaaaagatggatctacgacacaagcaaggtggctagcggcgtgccttaccggtttagcggctctggtagcggcacaagctatagcctcacgattagctcaatggaggccgaggacgccgccacgtattattgccaacagtggagcagcaaccctctgaccttcggcgccgggaccaagctggaacttaag

SEQ ID NO: 21 - protein, 487 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 22 - DNA, 1461 bp, synthetic construct
  source 1..1461: mol_type = other DNA
caagtgcagctggtgcagagcggcgtggaggtgaagaagccgggcgctagcgtgaaggtgagctgcaaggcctccggatataccttcacaaactactacatgtattgggttagacaagcccctggtcaaggcctggagtggatgggcggtataaacccttccaatggaggtactaatttcaacgaaaaattcaagaaccgtgtgaccctgaccaccgacagtagcaccaccactgcatacatggagctgaagagtctgcagttcgacgataccgcggtctactactgtgcccgacgagactatagatttgacatgggcttcgactactggggacaaggcactacggtgaccgtgagcagcggtggcggcggctccgaaattgtgctgacacagtcgcctgcaaccctgtccttgagccctggcgagcgggcaactctgagctgccgagctagcaaaggcgtgagtacaagcggctatagctacctacattggtatcagcaaaagcctggccaagccccgcgactgctgatctacctggctagctacctggagagcggcgttcctgctagattcagcggatctggctctggtactgacttcaccttaactatcagtagccttgaacccgaggacttcgctgtgtactattgtcagcacagcagagacctccctctgacctttggaggtggtaccaaggtcgaaatcaaaggaggcggagggtcgggcggtggtgggtctggaggtggaggaagccaagttcaactcgtgcagagcggtgtggaagttaaaaagcccggcgctagcgtaaaggtgagctgtaaggctagcggctacaccttcaccaattactacatgtactgggttaggcaagcccctggacaaggcttagaatggatgggtgggataaacccttcgaatggtggaaccaactttaacgagaagttcaaaaacagagtgacgttgaccaccgactctagcaccactaccgcgtacatggaactgaagagcctgcagttcgatgatacagcggtgtattattgcgcccgaagagactaccgattcgacatggggtttgactactggggccaaggcaccaccgtcaccgtcagcagcggcggaggcggtagtgagattgttctgacccaaagccctgccaccctctcgttaagcccgggggagagagcaaccctgagctgtagagctagcaagggcgtgagcacctcgggctacagttacctgcactggtaccaacagaagcctggccaagcccctcgattattgatctacttagctagctacttagaaagcggcgtgcccgctagattcagcggcagcgggagcggcaccgactttacactgacgataagtagcttagagcctgaggactttgcagtgtattactgccaacattcccgggacctgccgcttaccttcgggggcggcaccaaagtggagattaaa

SEQ ID NO: 23 - DNA, 399 bp, synthetic construct
  source 1..399: mol_type = other DNA
cagggccaggacaggcacatgatccggatgaggcagctcatcgacatcgtcgaccagctgaagaactacgtgaacgacctggtgcccgagtttctgcctgcccccgaggacgtggagaccaactgcgagtggtccgccttctcctgctttcagaaggcccagctgaagtccgccaacaccggcaacaacgagcggatcatcaacgtgagcatcaagaagctgaagcggaagcctccctccacaaacgccggcaggaggcagaagcacaggctgacctgccccagctgtgactcctacgagaagaagccccccaaggagttcctggagaggttcaagtccctgctgcagaagatgatccatcagcacctgtcctccaggacccacggctccgaggactcc

SEQ ID NO: 24 - protein, 133 aa, Homo sapiens [sequence omitted]

SEQ ID NO: 25 - protein, 133 aa, Homo sapiens
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 26 - protein, 79 aa, Homo sapiens
EGAVLPRSAKELRCQCIKTYSKPFHPKFIKELRVIESGPHCANTEIIVKLSDGRELCLDPKENWVQRVVEKFLKRAENS

SEQ ID NO: 27 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 28 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 29 - DNA, 918 bp, Homo sapiens
  source 1..918: mol_type = genomic DNA
atttgggaactgaagaaggacgtctacgtggtcgaactggactggtatcccgatgctcccggcgaaatggtggtgctcacttgtgacacccccgaagaagacggcatcacttggaccctcgatcagagcagcgaggtgctgggctccggaaagaccctcacaatccaagttaaggagttcggagacgctggccaatacacatgccacaagggaggcgaggtgctcagccattccttattattattacacaagaaggaagacggaatctggtccaccgacattttaaaagatcagaaggagcccaagaataagacctttttaaggtgtgaggccaaaaactacagcggtcgtttcacttgttggtggctgaccaccatttccaccgatttaaccttctccgtgaaaagcagccggggaagctccgaccctcaaggtgtgacatgtggagccgctaccctcagcgctgagagggttcgtggcgataacaaggaatacgagtacagcgtggagtgccaagaagatagcgcttgtcccgctgccgaagaatctttacccattgaggtgatggtggacgccgtgcacaaactcaagtacgagaactacacctcctccttctttatccgggacatcattaagcccgatcctcctaagaatttacagctgaagcctctcaaaaatagccggcaagttgaggtctcttgggaatatcccgacacttggagcacaccccacagctacttctctttaaccttttgtgtgcaagttcaaggtaaaagcaagcgggagaagaaagaccgggtgtttaccgacaaaaccagcgccaccgtcatctgtcggaagaacgcctccatcagcgtgagggctcaagatcgttattactccagcagctggtccgagtgggccagcgtgccttgttcc

SEQ ID NO: 30 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - DNA, 591 bp, Homo sapiens
  source 1..591: mol_type = genomic DNA
cgtaacctccccgtggctacccccgatcccggaatgttcccttgtttacaccacagccagaatttactgagggccgtgagcaacatgctgcagaaagctaggcagactttagaattttacccttgcaccagcgaggagatcgaccatgaagatatcaccaaggacaagacatccaccgtggaggcttgtttacctctggagctgacaaagaacgagtcttgtctcaactctcgtgaaaccagcttcatcacaaatggctcttgtttagcttcccggaagacctcctttatgatggctttatgcctcagctccatctacgaggatttaaagatgtaccaagtggagttcaagaccatgaacgccaagctgctcatggaccctaaacggcagatctttttagaccagaacatgctggctgtgattgatgagctgatgcaagctttaaacttcaactccgagaccgtccctcagaagtcctccctcgaggagcccgatttttacaagacaaagatcaaactgtgcattttactccacgcctttaggatccgggccgtgaccattgaccgggtcatgagctatttaaacgccagc

SEQ ID NO: 32 - protein, 518 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 33 - DNA, 1554 bp, Homo sapiens
  source 1..1554: mol_type = genomic DNA
atttgggaactgaagaaggacgtctacgtggtcgaactggactggtatcccgatgctcccggcgaaatggtggtgctcacttgtgacacccccgaagaagacggcatcacttggaccctcgatcagagcagcgaggtgctgggctccggaaagaccctcacaatccaagttaaggagttcggagacgctggccaatacacatgccacaagggaggcgaggtgctcagccattccttattattattacacaagaaggaagacggaatctggtccaccgacattttaaaagatcagaaggagcccaagaataagacctttttaaggtgtgaggccaaaaactacagcggtcgtttcacttgttggtggctgaccaccatttccaccgatttaaccttctccgtgaaaagcagccggggaagctccgaccctcaaggtgtgacatgtggagccgctaccctcagcgctgagagggttcgtggcgataacaaggaatacgagtacagcgtggagtgccaagaagatagcgcttgtcccgctgccgaagaatctttacccattgaggtgatggtggacgccgtgcacaaactcaagtacgagaactacacctcctccttctttatccgggacatcattaagcccgatcctcctaagaatttacagctgaagcctctcaaaaatagccggcaagttgaggtctcttgggaatatcccgacacttggagcacaccccacagctacttctctttaaccttttgtgtgcaagttcaaggtaaaagcaagcgggagaagaaagaccgggtgtttaccgacaaaaccagcgccaccgtcatctgtcggaagaacgcctccatcagcgtgagggctcaagatcgttattactccagcagctggtccgagtgggccagcgtgccttgttccggcggtggaggatccggaggaggtggctccggcggcggaggatctcgtaacctccccgtggctacccccgatcccggaatgttcccttgtttacaccacagccagaatttactgagggccgtgagcaacatgctgcagaaagctaggcagactttagaattttacccttgcaccagcgaggagatcgaccatgaagatatcaccaaggacaagacatccaccgtggaggcttgtttacctctggagctgacaaagaacgagtcttgtctcaactctcgtgaaaccagcttcatcacaaatggctcttgtttagcttcccggaagacctcctttatgatggctttatgcctcagctccatctacgaggatttaaagatgtaccaagtggagttcaagaccatgaacgccaagctgctcatggaccctaaacggcagatctttttagaccagaacatgctggctgtgattgatgagctgatgcaagctttaaacttcaactccgagaccgtccctcagaagtcctccctcgaggagcccgatttttacaagacaaagatcaaactgtgcattttactccacgcctttaggatccgggccgtgaccattgaccgggtcatgagctatttaaacgccagc

SEQ ID NO: 34 - protein, 120 aa, Mus sp.
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 35 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 36 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 37 - DNA, 471 bp, Homo sapiens
  source 1..471: mol_type = genomic DNA
tacttcggcaaactggaatccaagctgagcgtgatccggaatttaaacgaccaagttctgtttatcgatcaaggtaaccggcctctgttcgaggacatgaccgactccgattgccgggacaatgccccccggaccatcttcattatctccatgtacaaggacagccagccccggggcatggctgtgacaattagcgtgaagtgtgagaaaatcagcactttatcttgtgagaacaagatcatctcctttaaggaaatgaacccccccgataacatcaaggacaccaagtccgatatcatcttcttccagcggtccgtgcccggtcacgataacaagatgcagttcgaatcctcctcctacgagggctactttttagcttgtgaaaaggagagggatttattcaagctgatcctcaagaaggaggacgagctgggcgatcgttccatcatgttcaccgtccaaaacgaggat

SEQ ID NO: 38 - protein, 352 aa, Homo sapiens
RDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 39 - protein, 248 aa, Homo sapiens
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 40 - protein, 209 aa, Homo sapiens
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 41 - protein, 360 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 42 - protein, 361 aa, Homo sapiens
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTDFPYVSAAMPCFVIIIILCVPCCKKKTSAAEGPELVSLQVLDQHPVGTGDHRDAAQLGFQPLMSATGSTGSTEGA

SEQ ID NO: 43 - protein, 190 aa, Homo sapiens
WVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 44 - protein, 191 aa, Homo sapiens
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMS

SEQ ID NO: 45 - protein, 188 aa, Homo sapiens
DAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPG

SEQ ID NO: 46 - protein, 233 aa, Homo sapiens
HSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPDRWIILGAFILLVLMGIVLICVWWQNGEWQAGLWPLRTS

SEQ ID NO: 47 - protein, 193 aa, Homo sapiens
GLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSG

SEQ ID NO: 48 - protein, 193 aa, Homo sapiens
RRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSG

SEQ ID NO: 49 - DNA, 408 bp, Homo sapiens
  source 1..408: mol_type = genomic DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcacgatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgat

SEQ ID NO: 50 - DNA, 408 bp, synthetic construct
  source 1..408: mol_type = other DNA
atccccccccatgtgcaaaaatcagtgaacaatgacatgatcgtgaccgataataacggggcggtaaagtttcctcaactgtgtaaattttgcgacgtcagattcagcacatgcgacaatcaaaagtcctgcatgagcaactgcagcatcacctctatctgtgagaaaccacaagaagtatgtgtggcggtatggcggaagaacgatgaaaatatcacgctggagaccgtttgccatgatcctaagctgccatatcacgatttcatcctggaggacgccgctagccctaagtgtatcatgaaggagaagaagaagcccggtgagacctttttcatgtgctcatgcagctccgacgagtgcaacgacaacatcatcttcagcgaggagtacaatacaagcaatcctgac

SEQ ID NO: 51 - protein, 287 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 52 - DNA, 861 bp, synthetic construct
  source 1..861: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcacgatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgcacaatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 53 - protein, 229 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 54 - protein, 119 aa, synthetic construct [sequence omitted]

SEQ ID NO: 55 - protein, 107 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 56 - DNA, 357 bp, synthetic construct
  source 1..357: mol_type = other DNA
caagttcagctccagcaaagcggcgccgaactcgctcggcccggcgcttccgtgaagatgtcttgtaaggcctccggctataccttcacccggtacacaatgcactgggtcaagcaacggcccggtcaaggtttagagtggattggctatatcaacccctcccggggctataccaactacaaccagaagttcaaggacaaagccaccctcaccaccgacaagtccagcagcaccgcttacatgcagctgagctctttaacatccgaggattccgccgtgtactactgcgctcggtactacgacgatcattactgcctcgattactggggccaaggtaccaccttaacagtctcctcc

SEQ ID NO: 57 - DNA, 321 bp, synthetic construct
  source 1..321: mol_type = other DNA
cagatcgtgctgacccagtcccccgctattatgagcgctagccccggtgaaaaggtgactatgacatgcagcgccagctcttccgtgagctacatgaactggtatcagcagaagtccggcaccagccctaaaaggtggatctacgacaccagcaagctggccagcggcgtccccgctcactttcggggctccggctccggaacaagctactctctgaccatcagcggcatggaagccgaggatgccgctacctattactgtcagcagtggagctccaaccccttcacctttggatccggcaccaagctcgagattaatcgt

SEQ ID NO: 58 - protein, 241 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 59 - DNA, 723 bp, synthetic construct
  source 1..723: mol_type = other DNA
cagatcgtgctgacccagtcccccgctattatgagcgctagccccggtgaaaaggtgactatgacatgcagcgccagctcttccgtgagctacatgaactggtatcagcagaagtccggcaccagccctaaaaggtggatctacgacaccagcaagctggccagcggcgtccccgctcactttcggggctccggctccggaacaagctactctctgaccatcagcggcatggaagccgaggatgccgctacctattactgtcagcagtggagctccaaccccttcacctttggatccggcaccaagctcgagattaatcgtggaggcggaggtagcggaggaggcggatccggcggtggaggtagccaagttcagctccagcaaagcggcgccgaactcgctcggcccggcgcttccgtgaagatgtcttgtaaggcctccggctataccttcacccggtacacaatgcactgggtcaagcaacggcccggtcaaggtttagagtggattggctatatcaacccctcccggggctataccaactacaaccagaagttcaaggacaaagccaccctcaccaccgacaagtccagcagcaccgcttacatgcagctgagctctttaacatccgaggattccgccgtgtactactgcgctcggtactacgacgatcattactgcctcgattactggggccaaggtaccaccttaacagtctcctcc

SEQ ID NO: 60 - protein, 449 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 450 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 213 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 64 - protein, 236 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 65 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 66 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 68 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 163 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESV

SEQ ID NO: 73 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 195 bp, Homo sapiens
  source 1..195: mol_type = genomic DNA
attacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 76 - protein, 15 aa, synthetic construct
GGGGSGGGGSGGGGS

SEQ ID NO: 77 - DNA, 45 bp, synthetic construct
  source 1..45: mol_type = other DNA
ggcggtggaggatccggaggaggtggctccggcggcggaggatct

SEQ ID NO: 78 - protein, 8 aa, synthetic construct
GGGSGGGS

SEQ ID NO: 79 - protein, 4 aa, synthetic construct
GGGS

SEQ ID NO: 80 - protein, 18 aa, synthetic construct
MKWVTFISLLFLFSSAYS

SEQ ID NO: 81 - DNA, 54 bp, synthetic construct
  source 1..54: mol_type = other DNA
atgaaatgggtgacctttatttctttactgttcctctttagcagcgcctactcc

SEQ ID NO: 82 - DNA, 54 bp, synthetic construct
  source 1..54: mol_type = other DNA
atgaagtgggtcacatttatctctttactgttcctcttctccagcgcctacagc

SEQ ID NO: 83 - protein, 16 aa, synthetic construct
MKCLLYLAFLFLGVNC

SEQ ID NO: 84 - protein, 58 aa, synthetic construct
MGQIVTMFEALPHIIDEVINIVIIVLIIITSIKAVYNFATCGILALVSFLFLAGRSCG

SEQ ID NO: 85 - protein, 97 aa, synthetic construct
MPNHQSGSPTGSSDLLLSGKKQRPHLALRRKRRREMRKINRKVRRMNLAPIKEKTAWQHLQALISEAEEVLKTSQTPQNSLTLFLALLSVLGPPVTG

SEQ ID NO: 86 - protein, 30 aa, synthetic construct
MDSKGSSQKGSRLLLLLVVSNLLLCQGVVS

SEQ ID NO: 87 - protein, 15 aa, synthetic construct
GLNDIFEAQKIEWHE

SEQ ID NO: 88 - protein, 26 aa, synthetic construct
KRRWKKNFIAVSAANRFKKISSSGAL

SEQ ID NO: 89 - protein, 6 aa, synthetic construct
EEEEEE

SEQ ID NO: 90 - protein, 13 aa, synthetic construct
GAPVPYPDPLEPR

SEQ ID NO: 91 - protein, 8 aa, synthetic construct
DYKDDDDK

SEQ ID NO: 92 - protein, 9 aa, synthetic construct
YPYDVPDYA

SEQ ID NO: 93 - protein, 5 aa, synthetic construct
HHHHH

SEQ ID NO: 94 - protein, 6 aa, synthetic construct
HHHHHH

SEQ ID NO: 95 - protein, 7 aa, synthetic construct
HHHHHHH

SEQ ID NO: 96 - protein, 8 aa, synthetic construct
HHHHHHHH

SEQ ID NO: 97 - protein, 9 aa, synthetic construct
HHHHHHHHH

SEQ ID NO: 98 - protein, 10 aa, synthetic construct
HHHHHHHHHH

SEQ ID NO: 99 - protein, 10 aa, synthetic construct
EQKLISEEDL

SEQ ID NO: 100 - protein, 18 aa, synthetic construct
TKENPRSNQEESYDDNES

SEQ ID NO: 101 - protein, 15 aa, synthetic construct
KETAAAKFERQHMDS

SEQ ID NO: 102 - protein, 38 aa, synthetic construct
MDEKTTGWRGGHVVEGLAGELEQLRARLEHHPQGQREP

SEQ ID NO: 103 - protein, 13 aa, synthetic construct
SLAELLNAGLGGS

SEQ ID NO: 104 - protein, 8 aa, synthetic construct
TQDPSRVG

SEQ ID NO: 105 - protein, 12 aa, synthetic construct
PDRVRAVSHWSS

SEQ ID NO: 106 - protein, 8 aa, synthetic construct
WSHPQFEK

SEQ ID NO: 107 - protein, 6 aa, synthetic construct
CCPGCC

SEQ ID NO: 108 - protein, 10 aa, synthetic construct
EVHTNQDPLD

SEQ ID NO: 109 - protein, 14 aa, synthetic construct
GKPIPNPLLGLDST

SEQ ID NO: 110 - protein, 11 aa, synthetic construct
YTDIEMNRLGK

SEQ ID NO: 111 - protein, 8 aa, synthetic construct
DLYDDDDK

SEQ ID NO: 112 - protein, 10 aa, synthetic construct
EQKLISEEDL

SEQ ID NO: 113 - protein, 153 aa, Homo sapiens [sequence omitted]

SEQ ID NO: 114 - DNA, 831 bp, synthetic construct
  source 1..831: mol_type = other DNA
attcctcctcacgtgcagaaaagcgtgaacaacgacatgatagtgaccgacaataacggcgcggtcaagttcccacagctctgcaaattttgcgacgtgagatttagtacctgcgacaatcagaaatcctgcatgagcaattgtagcatcactagcatctgtgaaaagccccaagaggtctgcgtagcagtgtggagaaaaaatgacgagaacatcacactagagaccgtgtgccatgaccctaagctgccttatcatgatttcatccttgaagatgctgctagccctaagtgcattatgaaagagaaaaagaagcctggcgaaacctttttcatgtgtagctgtagtagcgacgagtgcaacgataacatcatcttctcggaggagtataatacatcgaatcctgatggcggcggcggcagcatccctcctcatgttcagaagagcgtgaataacgacatgattgtgacggataacaatggcgccgtaaagttccctcagctgtgtaagttctgcgatgtgcgatttagcacctgcgacaatcagaagagctgtatgtccaattgttcaatcacaagcatctgtgagaagcctcaagaggtgtgcgtggccgtttggagaaagaacgacgagaacattacccttgagaccgtatgccacgatcctaagctgccgtatcacgacttcatactggaggatgccgctagccctaaatgcatcatgaaagaaaaaaaaaagcctggtgagacattttttatgtgtagctgctcgtctgacgagtgcaatgacaatatcattttcagcgaagaatataacacctcaaaccctgac

SEQ ID NO: 115 - protein, 73 aa, synthetic construct
ITCPPPMSVEHADIWVKSYSLYSRERYICNSGFKRKAGTSSLTECVLNKATNVAHWTTPSLKCIRDPALVHQR

SEQ ID NO: 116 - DNA, 219 bp, synthetic construct
  source 1..219: mol_type = other DNA
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaagg

SEQ ID NO: 117 - protein, 493 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGS

SEQ ID NO: 118 - DNA, 1479 bp, synthetic construct
  source 1..1479: mol_type = other DNA
gacatacagatgacccaaagccctgctagcctgagcgcgagcgtgggcgacagagtgaccatcacctgcctcgcctctcagacgattgacacctggctggcttggtatttgcagaaacctgggaaaagccctcagctgctgatctacgcagccaccaatttagccgatggcgtcccttctagatttagcggctcgggatccggcaccgattttagctttacaatttcatctctacagcctgaggacttcgccacctattattgtcaacaagtatatagcagccctttcaccttcggccaaggcaccaagttagagatcaagggcggtggcggctccgggggcgggggaagtggaggtggggggtcacagattcagctggttcaaagcggcggcgaggtgaagaaacctggcgcctcggtgagagttagctgcaaagctagcggatattcgtttactgactacaacgtgtattgggtgaggcagagcccgggcaagggactggagtggattgggtacattgacccctacaacggcataaccatatatgatcagaacttcaagggcaaagccacgctgaccgtggataaaagcacaagcaccgcttatatggagttgagctccctccgtagcgaagacacggcagtatacttttgcgcacgggatgtgaccacggccctagatttctggggacaagggacaaccgttactgttagttccggcggtgggggctcggatattcagatgacgcagtcgccaagctcactctcggcctccgtaggagacagagttaccatcacctgcagagctagccaagacatcagaaactacctgaactggtatcagcagaagcctggcaaggcccctaagctcctgatctactacacgagcagacttgagagcggggtaccatcccgcttcagcggatctggaagcggcacagactacaccctgaccatctctagtctgcagcctgaggacttcgccacttactattgtcagcaaggcaacaccctgccttggacctttggccaaggcaccaaagttgagataaagggagggggagggagcggtggcggaggctcgggtgggggtggctcagaagtacagctggttgagtccggcggtggtctggtgcagcctggcggaagcctgagactgagctgcgccgcaagcggctatagcttcactggctataccatgaactgggtgcgccaagcccctggcaagggcctggaatgggtggccctgatcaacccttacaagggcgtgagcacctacaatcagaagttcaaggacagattcaccatcagcgtggacaagagcaagaacaccgcctacctgcagatgaacagcctgagagccgaggacactgcggtctattactgtgctagaagcggctactacggcgacagcgactggtacttcgacgtgtgggggcaaggcaccttggtaacggtaagctccggcggtgggggctcg

SEQ ID NO: 119 - protein, 240 aa, synthetic construct
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGHGGGGSGGGGSGGGGSEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 120 - DNA, 720 bp, synthetic construct
  source 1..720: mol_type = other DNA
agcgagctgacccaagaccctgccgtgagcgtggccctgggacagaccgtgagaatcacctgccaaggcgacagcctgagaagctactacgctagctggtatcagcagaagcctggccaagcccctgtgctggtgatctacggcaagaacaacagacctagcggcatccctgacagattcagcggcagcagcagcggcaacaccgctagcctgaccatcaccggcgcccaagccgaggacgaggccgactactactgcaacagcagagacagcagcggcaaccacgtggtcttcggcggaggaaccaaactcaccgtcggccacggcggaggcggaagcggcggcggaggctccggcggaggcggcagcgaagtgcagctggtggagagcggaggaggcgtcgtcagacctggcggcagcctgagactgagctgcgccgctagcggcttcaccttcgacgactacggcatgagctgggtgagacaagcccctggcaagggcctggagtgggtgagcggcatcaactggaacggcggcagcaccggctacgccgacagcgtgaagggcagattcaccatcagcagagacaacgccaagaacagcctgtacctgcagatgaacagcctgagagccgaggacaccgccgtgtactactgcgctagaggcagaagcctgctgttcgactactggggccaaggcaccctggtgaccgtgagcaga

SEQ ID NO: 121 - protein, 255 aa, synthetic construct
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSS

SEQ ID NO: 122 - DNA, 765 bp, synthetic construct
  source 1..765: mol_type = other DNA
tcttatgtaagaccgctctccgtcgcattgggggagaccgcgcgcattagttgtggacgacaagcacttggatctcgagcagttcagtggtaccagcaccgacccggacaagctcctattctgctcatttataataaccaagacagaccatcaggcatccccgagcgattctctgggaccccggatatcaatttcggaaccagggcgacacttactatttcaggtgttgaggccggggacgaggcagactattactgccacatgtgggacagccgatctggattttcttggtcatttggaggcgcaacacgcttgaccgttctcggagggggtggctcaggaggcggcggatcaggagggggaggttcacaagtgcaacttcaggaatccggacctggactcgtgaagccttcagaaaccctttcagtaacgtgcagcgtgagcggagatagcatgaacaactattactggacttggataagacaatctccgggcaaggggctcgagtggatagggtatatctcagatagagaatccgccacttataatcctagccttaacagccgggtagtcataagcagagacacaagcaaaaatcaactcagtttgaaattgaattcagtaacacctgccgacaccgcagtgtactattgcgcgacagcccggcgagggcagaggatttatggcgtcgttagctttggagaatttttctactattactccatggatgtgtggggcaaggggaccacggttactgtgtcttca

SEQ ID NO: 123 - DNA, 456 bp, synthetic construct
  source 1..456: mol_type = other DNA
gattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 124 - protein, 129 aa, Mus sp.
HKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 125 - DNA, 387 bp, synthetic construct
  source 1..387: mol_type = other DNA
cataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcc

SEQ ID NO: 126 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 127 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - DNA, 1614 bp, synthetic construct
  source 1..1614: mol_type = other DNA
atgggcgtgaaggtgctgttcgccctgatctgcatcgccgtggccgaggccattcctcctcacgtgcagaaaagcgtgaacaacgacatgatagtgaccgacaataacggcgcggtcaagttcccacagctctgcaaattttgcgacgtgagatttagtacctgcgacaatcagaaatcctgcatgagcaattgtagcatcactagcatctgtgaaaagccccaagaggtctgcgtagcagtgtggagaaaaaatgacgagaacatcacactagagaccgtgtgccatgaccctaagctgccttatcatgatttcatccttgaagatgctgctagccctaagtgcattatgaaagagaaaaagaagcctggcgaaacctttttcatgtgtagctgtagtagcgacgagtgcaacgataacatcatcttctcggaggagtataatacatcgaatcctgatggcggcggcggcagcatccctcctcatgttcagaagagcgtgaataacgacatgattgtgacggataacaatggcgccgtaaagttccctcagctgtgtaagttctgcgatgtgcgatttagcacctgcgacaatcagaagagctgtatgtccaattgttcaatcacaagcatctgtgagaagcctcaagaggtgtgcgtggccgtttggagaaagaacgacgagaacattacccttgagaccgtatgccacgatcctaagctgccgtatcacgacttcatactggaggatgccgctagccctaaatgcatcatgaaagaaaaaaaaaagcctggtgagacattttttatgtgtagctgctcgtctgacgagtgcaatgacaatatcattttcagcgaagaatataacacctcaaaccctgacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaacgtgatcagcgacctgaagaagatcgaggacctgattcagagcatgcacatcgacgccaccctgtacaccgagagcgacgtgcaccctagctgcaaggtgaccgccatgaagtgcttcctgctggagctgcaagtgatcagcctggagagcggcgacgctagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacacaagc

SEQ ID NO: 129 - protein, 117 aa, synthetic construct
QIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 130 - protein, 107 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIK

SEQ ID NO: 131 - protein, 239 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 132 - DNA, 717 bp, synthetic construct
  source 1..717: mol_type = other DNA
gacatacagatgacccaaagccctgctagcctgagcgcgagcgtgggcgacagagtgaccatcacctgcctcgcctctcagacgattgacacctggctggcttggtatttgcagaaacctgggaaaagccctcagctgctgatctacgcagccaccaatttagccgatggcgtcccttctagatttagcggctcgggatccggcaccgattttagctttacaatttcatctctacagcctgaggacttcgccacctattattgtcaacaagtatatagcagccctttcaccttcggccaaggcaccaagttagagatcaagggcggtggcggctccgggggcgggggaagtggaggtggggggtcacagattcagctggttcaaagcggcggcgaggtgaagaaacctggcgcctcggtgagagttagctgcaaagctagcggatattcgtttactgactacaacgtgtattgggtgaggcagagcccgggcaagggactggagtggattgggtacattgacccctacaacggcataaccatatatgatcagaacttcaagggcaaagccacgctgaccgtggataaaagcacaagcaccgcttatatggagttgagctccctccgtagcgaagacacggcagtatacttttgcgcacgggatgtgaccacggccctagatttctggggacaagggacaaccgttactgttagttcc

SEQ ID NO: 133 - protein, 238 aa, synthetic construct
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 134 - protein, 491 aa, synthetic construct
QVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELK

SEQ ID NO: 135 - protein, 246 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 136 - protein, 248 aa, synthetic construct
RTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSS

SEQ ID NO: 137 - protein, 17 aa, synthetic construct
MGVKVLFALICIAVAEA

SEQ ID NO: 138 - protein, 17 aa, synthetic construct
MERTLVCLVVIFLGTVA

SEQ ID NO: 139 - protein, 19 aa, synthetic construct
MGWSCIILFLVATATGVHS

SEQ ID NO: 140 - protein, 396 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 141 - protein, 829 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIRQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 142 - protein, 342 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 143 - protein, 833 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIRQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELK

SEQ ID NO: 144 - protein, 815 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIRDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 145 - protein, 593 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIRGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 146 - protein, 685 aa, synthetic construct
HKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSITCPPPMSVEHADIWVKSYSLYSRERYICNSGFKRKAGTSSLTECVLNKATNVAHWTTPSLKCIRQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELK

SEQ ID NO: 147 - protein, 198 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSITCPPPMSVEHADIWVKSYSLYSRERYICNSGFKRKAGTSSLTECVLNKATNVAHWTTPSLKCIR

SEQ ID NO: 148 - DNA, 1205 bp, synthetic construct
  source 1..1205: mol_type = other DNA
gattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgataacgcgtacgaag

SEQ ID NO: 149 - DNA, 611 bp, synthetic construct
  source 1..611: mol_type = other DNA
caaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagatgataacgcgtacgaag

SEQ ID NO: 150 - DNA, 1740 bp, synthetic construct
  source 1..1740: mol_type = other DNA
atgggctggagctgcatcatcctgttcctggtggccaccgccaccggcgtgcactccgacatacagatgacccaaagccctgctagcctgagcgcgagcgtgggcgacagagtgaccatcacctgcctcgcctctcagacgattgacacctggctggcttggtatttgcagaaacctgggaaaagccctcagctgctgatctacgcagccaccaatttagccgatggcgtcccttctagatttagcggctcgggatccggcaccgattttagctttacaatttcatctctacagcctgaggacttcgccacctattattgtcaacaagtatatagcagccctttcaccttcggccaaggcaccaagttagagatcaagggcggtggcggctccgggggcgggggaagtggaggtggggggtcacagattcagctggttcaaagcggcggcgaggtgaagaaacctggcgcctcggtgagagttagctgcaaagctagcggatattcgtttactgactacaacgtgtattgggtgaggcagagcccgggcaagggactggagtggattgggtacattgacccctacaacggcataaccatatatgatcagaacttcaagggcaaagccacgctgaccgtggataaaagcacaagcaccgcttatatggagttgagctccctccgtagcgaagacacggcagtatacttttgcgcacgggatgtgaccacggccctagatttctggggacaagggacaaccgttactgttagttccggcggtgggggctcgatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaagggatattcagatgacgcagtcgccaagctcactctcggcctccgtaggagacagagttaccatcacctgcagagctagccaagacatcagaaactacctgaactggtatcagcagaagcctggcaaggcccctaagctcctgatctactacacgagcagacttgagagcggggtaccatcccgcttcagcggatctggaagcggcacagactacaccctgaccatctctagtctgcagcctgaggacttcgccacttactattgtcagcaaggcaacaccctgccttggacctttggccaaggcaccaaagttgagataaagggagggggagggagcggtggcggaggctcgggtgggggtggctcagaagtacagctggttgagtccggcggtggtctggtgcagcctggcggaagcctgagactgagctgcgccgcaagcggctatagcttcactggctataccatgaactgggtgcgccaagcccctggcaagggcctggaatgggtggccctgatcaacccttacaagggcgtgagcacctacaatcagaagttcaaggacagattcaccatcagcgtggacaagagcaagaacaccgcctacctgcagatgaacagcctgagagccgaggacactgcggtctattactgtgctagaagcggctactacggcgacagcgactggtacttcgacgtgtgggggcaaggcaccttggtaacggtaagctcc

SEQ ID NO: 151 - protein, 671 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSITCPPPMSVEHADIWVKSYSLYSRERYICNSGFKRKAGTSSLTECVLNKATNVAHWTTPSLKCIRDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 152 - protein, 662 aa, synthetic construct
QGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSITCPPPMSVEHADIWVKSYSLYSRERYICNSGFKRKAGTSSLTECVLNKATNVAHWTTPSLKCIRDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 153 - protein, 464 aa, synthetic construct
MGVKVLFALICIAVAEAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSITCPPPMSVEHADIWVKSYSLYSRERYICNSGFKRKAGTSSLTECVLNKATNVAHWTTPSLKCIRGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSS

SEQ ID NO: 154 - protein, 447 aa, synthetic construct
HKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSITCPPPMSVEHADIWVKSYSLYSRERYICNSGFKRKAGTSSLTECVLNKATNVAHWTTPSLKCIRGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSS

SEQ ID NO: 155 - protein, 194 aa, synthetic construct
HKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSITCPPPMSVEHADIWVKSYSLYSRERYICNSGFKRKAGTSSLTECVLNKATNVAHWTTPSLKCIR

SEQ ID NO: 156 - protein, 521 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 157 - DNA, 1569 bp, synthetic construct
  source 1..1569: mol_type = other DNA
attcctcctcacgtgcagaaaagcgtgaacaacgacatgatagtgaccgacaataacggcgcggtcaagttcccacagctctgcaaattttgcgacgtgagatttagtacctgcgacaatcagaaatcctgcatgagcaattgtagcatcactagcatctgtgaaaagccccaagaggtctgcgtagcagtgtggagaaaaaatgacgagaacatcacactagagaccgtgtgccatgaccctaagctgccttatcatgatttcatccttgaagatgctgctagccctaagtgcattatgaaagagaaaaagaagcctggcgaaacctttttcatgtgtagctgtagtagcgacgagtgcaacgataacatcatcttctcggaggagtataatacatcgaatcctgatggcggcggcggcagcatccctcctcatgttcagaagagcgtgaataacgacatgattgtgacggataacaatggcgccgtaaagttccctcagctgtgtaagttctgcgatgtgcgatttagcacctgcgacaatcagaagagctgtatgtccaattgttcaatcacaagcatctgtgagaagcctcaagaggtgtgcgtggccgtttggagaaagaacgacgagaacattacccttgagaccgtatgccacgatcctaagctgccgtatcacgacttcatactggaggatgccgctagccctaaatgcatcatgaaagaaaaaaaaaagcctggtgagacattttttatgtgtagctgctcgtctgacgagtgcaatgacaatatcattttcagcgaagaatataacacctcaaaccctgacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaacgtgatcagcgacctgaagaagatcgaggacctgattcagagcatgcacatcgacgccaccctgtacaccgagagcgacgtgcaccctagctgcaaggtgaccgccatgaagtgcttcctgctggagctgcaagtgatcagcctggagagcggcgacgctagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacacaagctaatga

SEQ ID NO: 158 - DNA, 1256 bp, synthetic construct
  source 1..1256: mol_type = other DNA
atgggagtgaaagttctttttgcccttatttgtattgctgtggccgaggccgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgataacgcgtacgaag

SEQ ID NO: 159 - protein, 494 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIRDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 160 - protein, 595 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIRGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSS

SEQ ID NO: 161 - protein, 782 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSITCPPPMSVEHADIWVKSYSLYSRERYICNSGFKRKAGTSSLTECVLNKATNVAHWTTPSLKCIRDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 162 - protein, 558 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSITCPPPMSVEHADIWVKSYSLYSRERYICNSGFKRKAGTSSLTECVLNKATNVAHWTTPSLKCIR

SEQ ID NO: 163 - protein, 781 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSITCPPPMSVEHADIWVKSYSLYSRERYICNSGFKRKAGTSSLTECVLNKATNVAHWTTPSLKCIRDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 164 - protein, 776 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSITCPPPMSVEHADIWVKSYSLYSRERYICNSGFKRKAGTSSLTECVLNKATNVAHWTTPSLKCIRDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 165 - protein, 782 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSITCPPPMSVEHADIWVKSYSLYSRERYICNSGFKRKAGTSSLTECVLNKATNVAHWTTPSLKCIRDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 166 - protein, 561 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSITCPPPMSVEHADIWVKSYSLYSRERYICNSGFKRKAGTSSLTECVLNKATNVAHWTTPSLKCIRDPALVHQRDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 167 - DNA, 1188 bp, synthetic construct
  source 1..1188: mol_type = other DNA
gattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaacgtgatcagcgacctgaagaagatcgaggacctgattcagagcatgcacatcgacgccaccctgtacaccgagagcgacgtgcaccctagctgcaaggtgaccgccatgaagtgcttcctgctggagctgcaagtgatcagcctggagagcggcgacgctagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacacaagc

SEQ ID NO: 168 - DNA, 1683 bp, synthetic construct
  source 1..1683: mol_type = other DNA
gacatacagatgacccaaagccctgctagcctgagcgcgagcgtgggcgacagagtgaccatcacctgcctcgcctctcagacgattgacacctggctggcttggtatttgcagaaacctgggaaaagccctcagctgctgatctacgcagccaccaatttagccgatggcgtcccttctagatttagcggctcgggatccggcaccgattttagctttacaatttcatctctacagcctgaggacttcgccacctattattgtcaacaagtatatagcagccctttcaccttcggccaaggcaccaagttagagatcaagggcggtggcggctccgggggcgggggaagtggaggtggggggtcacagattcagctggttcaaagcggcggcgaggtgaagaaacctggcgcctcggtgagagttagctgcaaagctagcggatattcgtttactgactacaacgtgtattgggtgaggcagagcccgggcaagggactggagtggattgggtacattgacccctacaacggcataaccatatatgatcagaacttcaagggcaaagccacgctgaccgtggataaaagcacaagcaccgcttatatggagttgagctccctccgtagcgaagacacggcagtatacttttgcgcacgggatgtgaccacggccctagatttctggggacaagggacaaccgttactgttagttccggcggtgggggctcgatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaagggatattcagatgacgcagtcgccaagctcactctcggcctccgtaggagacagagttaccatcacctgcagagctagccaagacatcagaaactacctgaactggtatcagcagaagcctggcaaggcccctaagctcctgatctactacacgagcagacttgagagcggggtaccatcccgcttcagcggatctggaagcggcacagactacaccctgaccatctctagtctgcagcctgaggacttcgccacttactattgtcagcaaggcaacaccctgccttggacctttggccaaggcaccaaagttgagataaagggagggggagggagcggtggcggaggctcgggtgggggtggctcagaagtacagctggttgagtccggcggtggtctggtgcagcctggcggaagcctgagactgagctgcgccgcaagcggctatagcttcactggctataccatgaactgggtgcgccaagcccctggcaagggcctggaatgggtggccctgatcaacccttacaagggcgtgagcacctacaatcagaagttcaaggacagattcaccatcagcgtggacaagagcaagaacaccgcctacctgcagatgaacagcctgagagccgaggacactgcggtctattactgtgctagaagcggctactacggcgacagcgactggtacttcgacgtgtgggggcaaggcaccttggtaacggtaagctcc

SEQ ID NO: 169 - DNA, 1563 bp, synthetic construct
  source 1..1563: mol_type = other DNA
attcctcctcacgtgcagaaaagcgtgaacaacgacatgatagtgaccgacaataacggcgcggtcaagttcccacagctctgcaaattttgcgacgtgagatttagtacctgcgacaatcagaaatcctgcatgagcaattgtagcatcactagcatctgtgaaaagccccaagaggtctgcgtagcagtgtggagaaaaaatgacgagaacatcacactagagaccgtgtgccatgaccctaagctgccttatcatgatttcatccttgaagatgctgctagccctaagtgcattatgaaagagaaaaagaagcctggcgaaacctttttcatgtgtagctgtagtagcgacgagtgcaacgataacatcatcttctcggaggagtataatacatcgaatcctgatggcggcggcggcagcatccctcctcatgttcagaagagcgtgaataacgacatgattgtgacggataacaatggcgccgtaaagttccctcagctgtgtaagttctgcgatgtgcgatttagcacctgcgacaatcagaagagctgtatgtccaattgttcaatcacaagcatctgtgagaagcctcaagaggtgtgcgtggccgtttggagaaagaacgacgagaacattacccttgagaccgtatgccacgatcctaagctgccgtatcacgacttcatactggaggatgccgctagccctaaatgcatcatgaaagaaaaaaaaaagcctggtgagacattttttatgtgtagctgctcgtctgacgagtgcaatgacaatatcattttcagcgaagaatataacacctcaaaccctgacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaacgtgatcagcgacctgaagaagatcgaggacctgattcagagcatgcacatcgacgccaccctgtacaccgagagcgacgtgcaccctagctgcaaggtgaccgccatgaagtgcttcctgctggagctgcaagtgatcagcctggagagcggcgacgctagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacacaagc

SEQ ID NO: 170 - protein, 679 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSITCPPPMSVEHADIWVKSYSLYSRERYICNSGFKRKAGTSSLTECVLNKATNVAHWTTPSLKCIRDPALVHQRQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 171 - protein, 670 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSGGGGSGGGGSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSITCPPPMSVEHADIWVKSYSLYSRERYICNSGFKRKAGTSSLTECVLNKATNVAHWTTPSLKCIRDPALVHQRQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 172 - protein, 560 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSITCPPPMSVEHADIWVKSYSLYSRERYICNSGFKRKAGTSSLTECVLNKATNVAHWTTPSLKCIRGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 173 - protein, 557 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSITCPPPMSVEHADIWVKSYSLYSRERYICNSGFKRKAGTSSLTECVLNKATNVAHWTTPSLKCIRDPALVHQRSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGHGGGGSGGGGSGGGGSEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSR

SEQ ID NO: 174 - protein, 555 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSITCPPPMSVEHADIWVKSYSLYSRERYICNSGFKRKAGTSSLTECVLNKATNVAHWTTPSLKCIRDPALVHQRDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGGGGSGGGGSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 175 - protein, 580 aa, synthetic construct
MGWSCIILFLVATATGVHSDIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSITCPPPMSVEHADIWVKSYSLYSRERYICNSGFKRKAGTSSLTECVLNKATNVAHWTTPSLKCIRDPALVHQRDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 176 - protein, 562 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSITCPPPMSVEHADIWVKSYSLYSRERYICNSGFKRKAGTSSLTECVLNKATNVAHWTTPSLKCIRGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSS

SEQ ID NO: 177 - protein, 351 aa, synthetic construct
MGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSITCPPPMSVEHADIWVKSYSLYSRERYICNSGFKRKAGTSSLTECVLNKATNVAHWTTPSLKCIRQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 178 - protein, 334 aa, synthetic construct
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSITCPPPMSVEHADIWVKSYSLYSRERYICNSGFKRKAGTSSLTECVLNKATNVAHWTTPSLKCIRHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 179 - protein, 449 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSITCPPPMSVEHADIWVKSYSLYSRERYICNSGFKRKAGTSSLTECVLNKATNVAHWTTPSLKCIRQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 180 - protein, 685 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKITCPPPMSVEHADIWVKSYSLYSRERYICNSGFKRKAGTSSLTECVLNKATNVAHWTTPSLKCIRQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 181 - protein, 316 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSITCPPPMSVEHADIWVKSYSLYSRERYICNSGFKRKAGTSSLTECVLNKATNVAHWTTPSLKCIR

SEQ ID NO: 182 - protein, 552 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKITCPPPMSVEHADIWVKSYSLYSRERYICNSGFKRKAGTSSLTECVLNKATNVAHWTTPSLKCIR

SEQ ID NO: 183 - protein, 488 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTFGQGTKLEIKGGGGSGGGGSGGGGSQIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSSGGGGSEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 184 - protein, 468 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSITCPPPMSVEHADIWVKSYSLYSRERYICNSGFKRKAGTSSLTECVLNKATNVAHWTTPSLKCIRDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 185 - protein, 704 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKITCPPPMSVEHADIWVKSYSLYSRERYICNSGFKRKAGTSSLTECVLNKATNVAHWTTPSLKCIRDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 186 - DNA, 1580 bp, synthetic construct
  source 1..1580: mol_type = other DNA
atcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggcggaggtggaagtatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgataacgcgtacgaag

SEQ ID NO: 187 - DNA, 2115 bp, synthetic construct
  source 1..2115: mol_type = other DNA
caagttcaactcgtgcagagcggtgtggaagttaaaaagcccggcgctagcgtaaaggtgagctgtaaggctagcggctacaccttcaccaattactacatgtactgggttaggcaagcccctggacaaggcttagaatggatgggtgggataaacccttcgaatggtggaaccaactttaacgagaagttcaaaaacagagtgacgttgaccaccgactctagcaccactaccgcgtacatggaactgaagagcctgcagttcgatgatacagcggtgtattattgcgcccgaagagactaccgattcgacatggggtttgactactggggccaaggcaccaccgtcaccgtcagcagcggcggaggcggtagtgagattgttctgacccaaagccctgccaccctctcgttaagcccgggggagagagcaaccctgagctgtagagctagcaagggcgtgagcacctcgggctacagttacctgcactggtaccaacagaagcctggccaagcccctcgattattgatctacttagctagctacttagaaagcggcgtgcccgctagattcagcggcagcgggagcggcaccgactttacactgacgataagtagcttagagcctgaggactttgcagtgtattactgccaacattcccgggacctgccgcttaccttcgggggcggcaccaaagtggagattaaaggaggcggagggtcgggcggtggtgggtctggaggtggaggaagccaagtgcagctggtgcagagcggcgtggaggtgaagaagccgggcgctagcgtgaaggtgagctgcaaggcctccggatataccttcacaaactactacatgtattgggttagacaagcccctggtcaaggcctggagtggatgggcggtataaacccttccaatggaggtactaatttcaacgaaaaattcaagaaccgtgtgaccctgaccaccgacagtagcaccaccactgcatacatggagctgaagagtctgcagttcgacgataccgcggtctactactgtgcccgacgagactatagatttgacatgggcttcgactactggggacaaggcactacggtgaccgtgagcagcggtggcggcggctccgaaattgtgctgacacagtcgcctgcaaccctgtccttgagccctggcgagcgggcaactctgagctgccgagctagcaaaggcgtgagtacaagcggctatagctacctacattggtatcagcaaaagcctggccaagccccgcgactgctgatctacctggctagctacctggagagcggcgttcctgctagattcagcggatctggctctggtactgacttcaccttaactatcagtagccttgaacccgaggacttcgctgtgtactattgtcagcacagcagagacctccctctgacctttggaggtggtaccaaggtcgaaatcaaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 188 - protein, 708 aa, synthetic construct
QVQLQQSGAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKGGGGSGGGGSGGGGSQVQLQQSAAELVKPGSSVKISCKASGYTFTSHFIHWIKQQPGNGLEWIGGIYPGDGDTEYNQQFNGKATLTADKSSSTAYMRLSSLTSEDSAVYFCATRVPSYWFFDFWGPGTMVTVSSGGGGSRTDVALTQTPVAQPVTLGDQASISCRSSQSLVHSNGRTYLEWYLQKPGQSPQLLIYKVSNRFSGVPDRFIGSGSGSDFTLTISRVEPEDLGVYYCFQATHDPNTFGAGTKLELKITCPPPMSVEHADIWVKSYSLYSRERYICNSGFKRKAGTSSLTECVLNKATNVAHWTTPSLKCIRDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 189 - protein, 305 aa, synthetic construct
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVGHGGGGSGGGGSGGGGSEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRITCPPPMSVEHADIWVKSYSLYSRERYICNSGFKRKAGTSSLTECVLNKATNVAHWTTPSLKCIR

SEQ ID NO: 190 - protein, 577 aa, synthetic construct
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGGGGSGGGGSGGGGSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSGGGGSITCPPPMSVEHADIWVKSYSLYSRERYICNSGFKRKAGTSSLTECVLNKATNVAHWTTPSLKCIRDPALVHQRDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 191 - DNA, 1620 bp, synthetic construct
  source 1..1620: mol_type = other DNA
atgggcgtgaaggtgctgttcgccctgatctgcatcgccgtggccgaggccattcctcctcacgtgcagaaaagcgtgaacaacgacatgatagtgaccgacaataacggcgcggtcaagttcccacagctctgcaaattttgcgacgtgagatttagtacctgcgacaatcagaaatcctgcatgagcaattgtagcatcactagcatctgtgaaaagccccaagaggtctgcgtagcagtgtggagaaaaaatgacgagaacatcacactagagaccgtgtgccatgaccctaagctgccttatcatgatttcatccttgaagatgctgctagccctaagtgcattatgaaagagaaaaagaagcctggcgaaacctttttcatgtgtagctgtagtagcgacgagtgcaacgataacatcatcttctcggaggagtataatacatcgaatcctgatggcggcggcggcagcatccctcctcatgttcagaagagcgtgaataacgacatgattgtgacggataacaatggcgccgtaaagttccctcagctgtgtaagttctgcgatgtgcgatttagcacctgcgacaatcagaagagctgtatgtccaattgttcaatcacaagcatctgtgagaagcctcaagaggtgtgcgtggccgtttggagaaagaacgacgagaacattacccttgagaccgtatgccacgatcctaagctgccgtatcacgacttcatactggaggatgccgctagccctaaatgcatcatgaaagaaaaaaaaaagcctggtgagacattttttatgtgtagctgctcgtctgacgagtgcaatgacaatatcattttcagcgaagaatataacacctcaaaccctgacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaacgtgatcagcgacctgaagaagatcgaggacctgattcagagcatgcacatcgacgccaccctgtacaccgagagcgacgtgcaccctagctgcaaggtgaccgccatgaagtgcttcctgctggagctgcaagtgatcagcctggagagcggcgacgctagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacacaagctaatga

SEQ ID NO: 192 - DNA, 1239 bp, synthetic construct
  source 1..1239: mol_type = other DNA
atgggcgtgaaggtgctgttcgccctgatctgcatcgccgtggccgaggccgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaacgtgatcagcgacctgaagaagatcgaggacctgattcagagcatgcacatcgacgccaccctgtacaccgagagcgacgtgcaccctagctgcaaggtgaccgccatgaagtgcttcctgctggagctgcaagtgatcagcctggagagcggcgacgctagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacacaagc

SEQ ID NO: 193 - protein, 538 aa, synthetic construct
MGVKVLFALICIAVAEAIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 194 - protein, 360 aa, synthetic construct
MKWVTFISLLFLFSSAYSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 195 - DNA, 1631 bp, synthetic construct
  source 1..1631: mol_type = other DNA
atgggagtgaaagttctttttgcccttatttgtattgctgtggccgaggccatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggcggaggtggaagtatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgataacgcgtacgaag

SEQ ID NO: 196 - DNA, 2166 bp, synthetic construct
  source 1..2166: mol_type = other DNA
atgggagtgaaagttctttttgcccttatttgtattgctgtggccgaggcccaagttcaactcgtgcagagcggtgtggaagttaaaaagcccggcgctagcgtaaaggtgagctgtaaggctagcggctacaccttcaccaattactacatgtactgggttaggcaagcccctggacaaggcttagaatggatgggtgggataaacccttcgaatggtggaaccaactttaacgagaagttcaaaaacagagtgacgttgaccaccgactctagcaccactaccgcgtacatggaactgaagagcctgcagttcgatgatacagcggtgtattattgcgcccgaagagactaccgattcgacatggggtttgactactggggccaaggcaccaccgtcaccgtcagcagcggcggaggcggtagtgagattgttctgacccaaagccctgccaccctctcgttaagcccgggggagagagcaaccctgagctgtagagctagcaagggcgtgagcacctcgggctacagttacctgcactggtaccaacagaagcctggccaagcccctcgattattgatctacttagctagctacttagaaagcggcgtgcccgctagattcagcggcagcgggagcggcaccgactttacactgacgataagtagcttagagcctgaggactttgcagtgtattactgccaacattcccgggacctgccgcttaccttcgggggcggcaccaaagtggagattaaaggaggcggagggtcgggcggtggtgggtctggaggtggaggaagccaagtgcagctggtgcagagcggcgtggaggtgaagaagccgggcgctagcgtgaaggtgagctgcaaggcctccggatataccttcacaaactactacatgtattgggttagacaagcccctggtcaaggcctggagtggatgggcggtataaacccttccaatggaggtactaatttcaacgaaaaattcaagaaccgtgtgaccctgaccaccgacagtagcaccaccactgcatacatggagctgaagagtctgcagttcgacgataccgcggtctactactgtgcccgacgagactatagatttgacatgggcttcgactactggggacaaggcactacggtgaccgtgagcagcggtggcggcggctccgaaattgtgctgacacagtcgcctgcaaccctgtccttgagccctggcgagcgggcaactctgagctgccgagctagcaaaggcgtgagtacaagcggctatagctacctacattggtatcagcaaaagcctggccaagccccgcgactgctgatctacctggctagctacctggagagcggcgttcctgctagattcagcggatctggctctggtactgacttcaccttaactatcagtagccttgaacccgaggacttcgctgtgtactattgtcagcacagcagagacctccctctgacctttggaggtggtaccaaggtcgaaatcaaaatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 197 - DNA, 662 bp, synthetic construct
  source 1..662: mol_type = other DNA
atgggagtgaaagttctttttgcccttatttgtattgctgtggccgaggcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagatgataacgcgtacgaag

SEQ ID NO: 198 - protein, 413 aa, synthetic construct
MGVKVLFALICIAVAEADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS